(19) United States
(12) Patent Application Publication
(10) Pub. No.: US 2004/0249146 A1
(43) Pub. Date: Dec. 9, 2004

(54) Nucleic acid compositions conferring altered visual phenotypes

(76) Inventors: Vipula Kiran Shukla, Indianapolis, IN (US); Rodney Alan Crosley, Indianapolis, IN (US); Thomas Alan Skokut, Carmel, IN (US); Max Otto Ruegger, Inadianapolis, IN (US); Ignacio Mario Larrinua, Indianapolis, IN (US)

Correspondence Address: DOW AGROSCIENCES LLC, 9330 ZIONSVILLE RD, INDIANAPOLIS, IN, 46268, US

(21) Appl. No.: 10/487,801

(22) PCT Filed: Aug. 30, 2002
(86) PCT No.: PCT/US02/27880

(30) Foreign Application Priority Data
Aug. 31, 2001 (US) 60316326

(51) Int. Cl. A01H 1/00; C12N 15/82; C07H 21/04

(52) U.S. Cl. 536/23.6; 435/468; 800/278

(57) ABSTRACT

This invention describes the identification and isolation of genes that confer modifications of plant architecture and/or leaf surface features in plant. These genes are derived from the following sources: Nicotiana benthamiana, Arabidopsis thaliana, Oryzae sativa (var. Indica IR7), Papaver rhoeas, Saccharomyces cerivisiae and Trichoderma harzianum (Rifai 1295-22). Further, this invention also describes other both homologous and heterologous sequences with a high degree of functional similarities.

### FIELD OF THE INVENTION

This invention relates to nucleic acid and amino acid sequences that confer altered visual phenotypes in plants, as well as plants, plant seeds, plant tissues and plant cells comprising such sequences.

### BACKGROUND OF THE INVENTION

It is believed that domestication of plants took place some 10,000 years ago when the first farmer gathered seed from a plant in the wild that attracted his attention because it exhibited a certain trait that he would like to reproduce and use for food. Farming has changed considerably over the years but the farmer is still looking for traits that will give him a better crop with respect to yield, stand, controlled harvest, pest protection and numerous other traits.

New traits in crop plants are discovered and introduced into crop plants by various methods. Traditional breeding can take new traits observed in wild relatives of crop plants, or discovered by crosses from individuals within a particular species, and introduce these traits into the crop of choice by various crosses and back-crosses. New traits can also be discovered using procedures that cause mutations in an individual crop plant. If the resultant mutation is a desirable trait it too can be introduced into a crop line by breeding.

Another method is that of genetic engineering which can create a new trait by the introduction of a new gene or genes into crop plants. These genes can come from any organism; plant, animal or microorganism. One of the goals of genetic engineering is to increase crop yields. For example, herbicide-tolerant traits make crops resistant to a given herbicide allowing farmers to time their use of herbicides thus increasing the effectiveness of the herbicide. Other traits make it possible for plants to resist insect pests. The advantage of pest-resistant crops is two fold. Control of target pests and a reduction in the use of costly chemical control. It has been estimated that total insecticide use in cotton in 1998 was around 1,000 tons less than that used before B.t. cotton was introduced. Still, other traits can help crops resist the impact of plant pathogens. The molecular description of resistance genes should enable them to be moved more rapidly into crops. It should also enable a range of different resistance genes to be assembled in different lines of the same cultivar so as to allow mosaics of resistance genes to be used within a single field (Miflin, B. J. 2000). Water is probably the crop resource that is in shortest supply and this condition will only worsen. The increased use of irrigation leads to changes in the soil creating the potential for additional abiotic stresses. Traits that enable crops to tolerate abiotic stress, such as drought and high or low pH, allow farmers to plant crops in marginal soils and sustain yields during unfavorable growing conditions.

Additionally, traits can help increase the yield and/or value of a crop by helping to reduce crop moisture or by making it easier to process. Genetic engineering can make it possible to transform crops in several different ways. For instance it is possible to alter the natural mix between oil and meal in a crop. Genetic engineering can make it possible to increase the solid content of a crop and can be used to modify the ripening process, increase the starch content of crops, and it can even create new molecules with health-related benefits. These benefits can end up in a variety of goods from oil or low saturated fat products to new pharmaceutical entities. Genetically engineered traits can also lead to crops that can be used for a variety of high-value goods including modified oils and enzymes.

Accordingly, what is needed in the art is the identification of gene sequences and polypeptide sequences whose expression causes desired traits in plants.

### SUMMARY OF THE INVENTION

This invention relates to nucleic acid and amino acid sequences that confer altered visual phenotypes in plants, as well as plants, plant seeds, plant tissues and plant cells comprising such sequences. In some embodiments, the present invention provides polynucleotides and polypeptides that confer altered visual phenotypes when expressed in plants. The present invention is not limited to any particular altered visual phenotype. Indeed, the introduction of variety of altered visual phenotypes is contemplated, including, but not limited to chlorotic, bleaching, etching, wilting, necrosis, auxin response, dark green, gray leaf, wet leaf, fluorescent, stunting, chlorotic etching, elongation, and texture phenotypes and combinations thereof. The present invention is not limited to any particular polypeptide or polynucleotide sequences that confer altered visual phenotypes. Indeed, a variety of such sequences are contemplated. Accordingly, in some embodiments the present invention provides an isolated nucleic acid selected from the group consisting of SEQ ID NOs: 1-2065 and nucleic acid sequences that hybridize to any thereof under conditions of low stringency, wherein expression of the isolated nucleic acid in a plant results in an altered visual phenotype. In further preferred embodiments, the present invention provides vectors comprising the foregoing polynucleotide sequences. In still further embodiments, the foregoing sequences are operably linked to an exogenous promoter, most preferably a plant promoter. However, the present invention is not limited to the use of any particular promoter. Indeed, the use of a variety of promoters is contemplated, including, but not limited to, 35S and 19S of Cauliflower Mosaic virus, Cassava Vein Mosaic virus, ubiquitin, heat shock and rubisco promoters. In some embodiments, the nucleic acid sequences of the present invention are arranged in sense orientation, while in other embodiments, the nucleic acid sequences are arranged in the vector in antisense orientation. In still further embodiments, the present invention provides a plant comprising one of the foregoing nucleic acid sequences or vectors, as well as seeds, leaves, and fruit from the plant. In some particularly preferred embodiments, the present invention provides at least one of the foregoing sequences for use in providing an altered visual phenotype in a plant.

In still other embodiments, the present invention provides processes for making a transgenic plant comprising providing a vector as described above and a plant, and transfecting the plant with the vector. In other preferred embodiments, the present invention provides processes for providing an altered visual phenotype in a plant or population of plants comprising providing a vector as described above and a plant, and transfecting the plant with the vector under conditions such that an altered visual phenotype is conferred by expression of the isolated nucleic acid from the vector. In still further embodiments, the present invention provides an isolated nucleic acid selected from the group consisting of SEQ ID NOs: 1-2065 and nucleic acid sequences that hybridize to any thereof under conditions of low stringency for use in producing a plant with an altered visual phenotype. In other embodiments, the present invention provides an isolated nucleic acid, composition or vector substantially as described herein in any of the examples or claims.

### DESCRIPTION OF THE DRAWINGS

FIG. 1 presents the contig sequences corresponding to SEQ ID NOs:1-311 and 2024-2065.

FIG. 2 presents homologous sequences SEQ ID NOs: 312-2023.

FIG. 3 is a table of blast search results from public databases.

FIG. 4 is a table of blast search results from the Derwent amino acid database.

FIG. 5 is a table of blast search results from the Derwent nucleotide database.

FIG. 6 is a table summarizing the results of the altered visual phenotype screen.

FIG. 7 is a table summarizing the results of an altered visual phenotype screen of representative homologs.

### DEFINITIONS

Before the present proteins, nucleotide sequences, and methods are described, it should be noted that this invention is not limited to the particular methodology, protocols, cell lines, vectors, and reagents described herein as these may vary. It should also be understood that the terminology used herein is for the purpose of describing particular aspects of the invention, and is not intended to limit its scope, which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies that are reported in the publications that might be used in connection with the invention. Nothing herein is to be construed as an admission that the invention is not entitled to antedate such disclosure by virtue of prior invention.

“Acylate”, as used herein, refers to the introduction of an acyl group into a molecule, (for example, acylation).

“Adjacent”, as used herein, refers to a position in a nucleotide sequence immediately 5′ or 3′ to a defined sequence.

“Agonist”, as used herein, refers to a molecule which, when bound to a polypeptide (for example, a polypeptide encoded by a nucleic acid of the present invention), increases the biological or immunological activity of the polypeptide. Agonists may include proteins, nucleic acids, carbohydrates, or any other molecules that bind to the protein.

“Alterations” in a polynucleotide (for example, a polypeptide encoded by a nucleic acid of the present invention), as used herein, comprise any deletions, insertions, and point mutations in the polynucleotide sequence. Included within this definition are alterations to the genomic DNA sequence that encodes the polypeptide.

“Amino acid sequence”, as used herein, refers to an oligopeptide, peptide, polypeptide, or protein sequence, and fragments or portions thereof, and to naturally occurring or synthetic molecules. “Amino acid sequence” and like terms, such as “polypeptide” or “protein” as recited herein are not meant to limit the amino acid sequence to the complete, native amino acid sequence associated with the recited protein molecule.

“Amplification”, as used herein, refers to the production of additional copies of a nucleic acid sequence and is generally carried out using polymerase chain reaction (PCR) technologies well known in the art (Dieffenbach, C. W. and G. S. Dveksler (1995) PCR Primer, a Laboratory Manual, Cold Spring Harbor Press, Plainview, N.Y.).

“Antibody” refers to intact molecules as well as fragments thereof that are capable of specific binding to a epitopic determinant. Antibodies that bind a polypeptide (for example, a polypeptide encoded by a nucleic acid of the present invention) can be prepared using intact polypeptides or fragments as the immunizing antigen. These antigens may be conjugated to a carrier protein, if desired.

“Antigenic determinant”, “determinant group”, or “epitope of an antigenic macromolecule”, as used herein, refer to any region of the macromolecule with the ability or potential to elicit, and combine with, one or more specific antibodies. Determinants exposed on the surface of the macromolecule are likely to be immunodominant, that is, more immunogenic than other (immunorecessive) determinants that are less exposed, while some (for example, those within the molecule) are non-immunogenic (immunosilent). As used herein, “antigenic determinant” refers to that portion of a molecule that makes contact with a particular antibody (for example, an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (the immunogen used to elicit the immune response) for binding to an antibody.

“Antisense”, as used herein, refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, for example, at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases.

“Anti-sense inhibition”, as used herein, refers to a type of gene regulation based on cytoplasmic, nuclear, or organelle inhibition of gene expression due to the presence in a cell of an RNA molecule complementary to at least a portion of the mRNA being translated. It is specifically contemplated that DNA molecules may be from either an RNA virus or mRNA from the host cell genome or from a DNA virus.

“Antagonist” or “inhibitor”, as used herein, refer to a molecule that, when bound to a polypeptide (for example, a polypeptide encoded by a nucleic acid of the present invention), decreases the biological or immunological activity of the polypeptide. Antagonists and inhibitors may include proteins, nucleic acids, carbohydrates, or any other molecules that bind to the polypeptide.

“Biologically active”, as used herein, refers to a molecule having the structural, regulatory, or biochemical functions of a naturally occurring molecule.

“Cell culture”, as used herein, refers to a proliferating mass of cells that may be in either an undifferentiated or differentiated state.

“Chimeric plasmid”, as used herein, refers to any recombinant plasmid formed (by cloning techniques) from nucleic acids derived from organisms that do not normally exchange genetic information (for example, Escherichia coli and Saccharomyces cerevisiae).

“Chimeric sequence” or “chimeric gene”, as used herein, refer to a nucleotide sequence derived from at least two heterologous parts. The sequence may comprise DNA or RNA.

“Coding sequence”, as used herein, refers to a deoxyribonucleotide sequence that, when transcribed and translated, results in the formation of a cellular polypeptide or a ribonucleotide sequence that, when translated, results in the formation of a cellular polypeptide.

“Compatible”, as used herein, refers to the capability of operating with other components of a system. A vector or plant viral nucleic acid that is compatible with a host is one that is capable of replicating in that host. A coat protein that is compatible with a viral nucleotide sequence is one capable of encapsidating that viral sequence.

“Coding region”, as used herein, refers to that portion of a gene that codes for a protein. The term “non-coding region” refers to that portion of a gene that is not a coding region.

“Complementary” or “complementarity”, as used herein, refer to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to it's complementary base. The degree of complementarity between nucleic acid strands has significant effects on the efficiency and strength of hybridization between nucleic acid strands.

“Contig” refers to a nucleic acid sequence that is derived from the contiguous assembly of two or more nucleic acid sequences.

“Correlates with expression of a polynucleotide”, as used herein, indicates that the detection of the presence of ribonucleic acid that is similar to a nucleic acid (for example, SEQ ID NOs:1-2065) and is indicative of the presence of mRNA encoding a polypeptide (for example, a polypeptide encoded by a nucleic acid of the present invention) in a sample and thereby correlates with expression of the transcript from the polynucleotide encoding the protein.

“Deletion”, as used herein, refers to a change made in either an amino acid or nucleotide sequence resulting in the absence of one or more amino acids or nucleotides, respectively.

“Encapsidation”, as used herein, refers to the process during virion assembly in which nucleic acid becomes incorporated in the viral capsid or in a head/capsid precursor (for example, in certain bacteriophages).

“Exon”, as used herein, refers to a polynucleotide sequence in a nucleic acid that encodes information for protein synthesis and that is copied and spliced together with other such sequences to form messenger RNA.

“Expression”, as used herein, is meant to incorporate transcription, reverse transcription, and translation.

“Expressed sequence tag (EST)” as used herein, refers to relatively short single-pass DNA sequences obtained from one or more ends of cDNA clones and RNA derived therefrom. They may be present in either the 5′ or the 3′ orientation. ESTs have been shown to be useful for identifying particular genes.

“Industrial crop”, as used herein, refers to crops grown primarily for consumption by humans or animals or use in industrial processes (for example, as a source of fatty acids for manufacturing or sugars for producing alcohol). It will be understood that either the plant or a product produced from the plant (for example, sweeteners, oil, flour, or meal) can be consumed. Examples of food crops include, but are not limited to, corn, soybean, rice, wheat, oilseed rape, cotton, oats, barley, and potato plants.

“Foreign gene”, as used herein, refers to any sequence that is not native to the organism.

“Fusion protein”, as used herein, refers to a protein containing amino acid sequences from each of two distinct proteins; it is formed by the expression of a recombinant gene in which two coding sequences have been joined together such that their reading frames are in phase. Hybrid genes of this type may be constructed in vitro in order to label the product of a particular gene with a protein that can be more readily assayed (for example, a gene fused with lacZ in E. coli to obtain a fusion protein with β-galactosidase activity). As a non-limiting second example, a fusion protein may comprise a protein linked to a signal peptide to allow its secretion by the cell. The products of certain viral oncogenes are fusion proteins.

“Gene”, as used herein, refers to a discrete nucleic acid sequence responsible for a discrete cellular product. The term “gene”, as used herein, refers not only to the nucleotide sequence encoding a specific protein, but also to any adjacent 5′ and 3′ non-coding nucleotide sequence involved in the regulation of expression of the protein encoded by the gene of interest. These non-coding sequences include terminator sequences, promoter sequences, upstream activator sequences, regulatory protein binding sequences, and the like. These non-coding sequence gene regions may be readily identified by comparison with previously identified eukaryotic non-coding sequence gene regions. Furthermore, the person of average skill in the art of molecular biology is able to identify the nucleotide sequences forming the non-coding regions of a gene using well-known techniques such as a site-directed mutagenesis, sequential deletion, promoter probe vectors, and the like.

“Growth cycle”, as used herein, is meant to include the replication of a nucleus, an organelle, a cell, or an organism.

The term “heterologous gene”, as used herein, means a gene encoding a protein, polypeptide, RNA, or a portion of any thereof, whose exact amino acid sequence is not normally found in the host cell, but is introduced by standard gene transfer techniques.

“Host”, as used herein, refers to a cell, tissue or organism capable of replicating a vector or plant viral nucleic acid and that is capable of being infected by a virus containing the viral vector or plant viral nucleic acid. This term is intended to include prokaryotic and eukaryotic cells, organs, tissues or organisms, where appropriate.

The term “homolog” as in a “homolog” of a given nucleic acid sequence, refers to a nucleic acid sequence (for example, a nucleic acid sequence from another organism), that shares a given degree of “homology” with the nucleic acid sequence.

“Homology” refers to a degree of complementarity. There may be partial homology or complete homology (identity). A partially complementary sequence is one that at least partially inhibits a completely complementary sequence from hybridizing to a target nucleic acid and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (the hybridization) of a completely homologous sequence to a target under conditions of low stringency. This is not to say that conditions of low stringency are such that non-specific binding is permitted; low stringency conditions require that the binding of two sequences to one another be a specific (selective) interaction. The absence of non-specific binding may be tested by the use of a second target that lacks even a partial degree of complementarity (for example, less than about 30% identity); in the absence of non-specific binding the probe will not hybridize to the second non-complementary target.

Numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (for example, the presence or absence of formamide, dextran sulfate, polyethylene glycol) are considered and the hybridization solution may be varied to generate conditions of low stringency hybridization different from, but equivalent to, the above listed conditions. In addition, conditions that promote hybridization under conditions of high stringency (for example, increasing the temperature of the hybridization and/or wash steps, the use of formamide in the hybridization solution, etc.) are readily apparent to one skilled in the art.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity, they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (it is the complement of) the single-stranded nucleic acid sequence under conditions of low stringency as described above.

The term “hybridization” is used in reference to the pairing of complementary nucleic acids. Hybridization and the strength of hybridization (for example, the strength of the association between the nucleic acids) is impacted by such factors as the degree of complementary between the nucleic acids, stringency of the conditions involved, the melting temperature (Tₘ) of the formed hybrid, and the G:C ratio within the nucleic acids.

“Hybridization complex”, as used herein, refers to a complex formed between nucleic acid strands by virtue of hydrogen bonding, stacking or other non-covalent interactions between bases. A hybridization complex may be formed in solution or between nucleic acid sequences present in solution and nucleic acid sequences immobilized on a solid support (for example, membranes, filters, chips, pins or glass slides to which cells have been fixed for in situ hybridization).

“Immunologically active” refers to the capability of a natural, recombinant, or synthetic polypeptide, or any oligopeptide thereof, to bind with specific antibodies and induce a specific immune response in appropriate animals or cells.

“Induction” and the terms “induce”, “induction” and “inducible”, as used herein, refer generally to a gene and a promoter operably linked thereto which is in some manner dependent upon an external stimulus, such as a molecule, in order to actively transcribed and/or translate the gene.

“Infection”, as used herein, refers to the ability of a virus to transfer its nucleic acid to a host or introduce viral nucleic acid into a host, wherein the viral nucleic acid is replicated, viral proteins are synthesized, and new viral particles assembled. In this context, the terms “transmissible” and “infective” are used interchangeably herein.

“Insertion” or “addition”, as used herein, refers to the replacement or addition of one or more nucleotides or amino acids, to a nucleotide or amino acid sequence, respectively.

“In cis”, as used herein, indicates that two sequences are positioned on the same strand of RNA or DNA.

“In trans”, as used herein, indicates that two sequences are positioned on different strands of RNA or DNA.

“Intron”, as used herein, refers to a polynucleotide sequence in a nucleic acid that does not encode information for protein synthesis and is removed before translation of messenger RNA.

“Isolated”, as used herein, refers to a polypeptide or polynucleotide molecule separated not only from other peptides, DNAs, or RNAs, respectively, that are present in the natural source of the macromolecule. “Isolated” and “purified” do not encompass either natural materials in their native state or natural materials that have been separated into components (for example, in an acrylamide gel) but not obtained either as pure substances or as solutions.

“Kinase”, as used herein, refers to an enzyme (for example, hexokinase and pyruvate kinase) that catalyzes the transfer of a phosphate group from one substrate (commonly ATP) to another.

“Marker” or “genetic marker”, as used herein, refer to a genetic locus that is associated with a particular, usually readily detectable, genotype or phenotypic characteristic (for example, an antibiotic resistance gene).

“Metabolome”, as used herein, indicates the complement of relatively low molecular weight molecules that is present in a plant, plant part, or plant sample, or in a suspension or extract thereof. Examples of such molecules include, but are not limited to: acids and related compounds; mono-, di-, and tri-carboxylic acids (saturated, unsaturated, aliphatic and cyclic, aryl, alkaryl); aldo-acids, keto-acids; lactone forms; gibberellins; abscisic acid; alcohols, polyols, derivatives, and related compounds; ethyl alcohol, benzyl alcohol, methanol; propylene glycol, glycerol, phytol; inositol, furfuryl alcohol, menthol; aldehydes, ketones, quinones, derivatives, and related compounds; acetaldehyde, butyraldehyde, benzaldehyde, acrolein, furfural, glyoxal; acetone, butanone; anthraquinone; carbohydrates; mono-, di-, tri-saccbarides; alkaloids, amines, and other bases; pyridines (including nicotinic acid, nicotinamide); pyrimidines (including cytidine, thymine); purines (including guanine, adenine, xanthines/hypoxanthines, kinetin); pyrroles; quinolines (including isoquinolines); morphinans, tropanes, cinchonans; nucleotides, oligonucleotides, derivatives, and related compounds; guanosine, cytosine, adenosine, thymidine, inosine; amino acids, oligopeptides, derivatives, and related compounds; esters; phenols and related compounds; heterocyclic compounds and derivatives; pyrroles, tetrapyrroles (corrinoids and porphines/porphyrins, w/w/o metal-ion); flavonoids; indoles; lipids (including fatty acids and triglycerides), derivatives, and related compounds; carotenoids, phytoene; and sterols, isoprenoids including terpenes.

“Modulate”, as used herein, refers to a change or an alteration in the biological activity of a polypeptide (for example, a polypeptide encoded by a nucleic acid of the present invention). Modulation may be an increase or a decrease in protein activity, a change in binding characteristics, or any other change in the biological, functional or immunological properties of the polypeptide.

“Movement protein”, as used herein, refers to a noncapsid protein required for cell to cell movement of replicons or viruses in plants.

“Multigene family”, as used herein, refers to a set of genes descended by duplication and variation from some ancestral gene. Such genes may be clustered together on the same chromosome or dispersed on different chromosomes. Examples of multigene families include those which encode the histones, hemoglobins, immunoglobulins, histocompatibility antigens, actins, tubulins, keratins, collagens, heat shock proteins, salivary glue proteins, chorion proteins, cuticle proteins, yolk proteins, and phaseolins.

“Nucleic acid sequence”, as used herein, refers to a polymer of nucleotides in which the 3′ position of one nucleotide sugar is linked to the 5′ position of the next by a phosphodiester bridge. In a linear nucleic acid strand, one end typically has a free 5′ phosphate group, the other a free 3′ hydroxyl group. Nucleic acid sequences may be used herein to refer to oligonucleotides, or polynucleotides, and fragments or portions thereof, and to DNA or RNA of genomic or synthetic origin that may be single- or double-stranded, and represent the sense or antisense strand.

“Polypeptide”, as used herein, refers to an amino acid sequence obtained from any species and from any source whether natural, synthetic, semi-synthetic, or recombinant.

“Oil-producing species,” as used herein, refers to plant species that produce and store triacylglycerol in specific organs, primarily in seeds. Such species include soybean (Glycine max), rapeseed and canola (including Brassica napus, Brassica rapa and Brassica campestris), sunflower (Helianthus annus), cotton (Gossypium hirsutum), corn (Zea mays), cocoa (Theobroma cacao), safflower (Carthamus tinctorius), oil palm (Elaeis guineensis), coconut palm (Cocos nucifera), flax (Linum usitatissimum), castor (Ricinus communis) and peanut (Arachis hypogaea). The group also includes non-agronomic species that are useful in developing appropriate expression vectors such as tobacco, rapid cycling Brassica species, and Arabidopsis thaliana, and wild species that may be a source of unique fatty acids.

“Operably linked” refers to ajuxtaposition of components, particularly nucleotide sequences, such that the normal function of the components can be performed. Thus, a coding sequence that is operably linked to regulatory sequences refers to a configuration of nucleotide sequences wherein the coding sequences can be expressed under the regulatory control, that is, transcriptional and/or translational control, of the regulatory sequences.

“Origin of assembly”, as used herein, refers to a sequence where self-assembly of the viral RNA and the viral capsid protein initiates to form virions.

“Ortholog” refers to genes that have evolved from an ancestral locus.

“Overexpression” refers to the production of a gene product in transgenic organisms that exceeds levels of production in normal or non-transformed organisms.

“Cosuppression” refers to the expression of a foreign gene that has substantial homology to an endogenous gene resulting in the suppression of expression of both the foreign and the endogenous gene. As used herein, the term “altered levels” refers to the production of gene product(s) in transgenic organisms in amounts or portions that differ from that of normal or non-transformed organisms.

“Phenotype” or “phenotypic trait(s)”, as used herein, refers to an observable property or set of properties resulting from the expression of a gene. “Visual phenotype”, as used herein, refers to a plant displaying a symptom or group of symptoms that meet defined criteria. “Altered visual phenotype” as used herein refers a plant that visually displays a symptom or group of symptoms that differ from those displayed by a wild-type plant. Examples of altered visual phenotypes include, but are not limited to, chlorotic, bleaching, etching, wilting, necrosis, auxin response, dark green, gray leaf, wet leaf, fluorescent, and texture phenotypes and combinations thereof.

“Plant”, as used herein, refers to any plant and progeny thereof. The term also includes parts of plants, including seed, cuttings, tubers, fruit, flowers, etc. In a preferred embodiment, “plant” refers to cultivated plant species, such as corn, cotton, canola, sunflower, soybeans, sorghum, alfalfa, wheat, rice, plants producing fruits and vegetables, and turf and ornamental plant species.

“Plant cell”, as used herein, refers to the structural and physiological unit of plants, consisting of a protoplast and the cell wall.

“Plant organ”, as used herein, refers to a distinct and visibly differentiated part of a plant, such as root, stem, leaf or embryo.

“Plant tissue”, as used herein, refers to any tissue of a plant in planta or in culture. This term is intended to include a whole plant, plant cell, plant organ, protoplast, cell culture, or any group of plant cells organized into a structural and functional unit.

“Portion”, as used herein, with regard to a protein (“a portion of a given protein”) refers to fragments of that protein. The fragments may range in size from four amino acid residues to the entire amino acid sequence minus one amino acid (10 nucleotides, 20, 30, 40, 50, 100, 200, etc.). A “portion” is preferably at least 25 nucleotides, more preferably at least 50 nucleotides, and even more preferably at least 100 nucleotides.

“Positive-sense inhibition”, as used herein, refers to a type of gene regulation based on cytoplasmic inhibition of gene expression due to the presence in a cell of an RNA molecule substantially homologous to at least a portion of the mRNA being translated.

“Production cell”, as used herein, refers to a cell, tissue or organism capable of replicating a vector or a viral vector, but which is not necessarily a host to the virus. This term is intended to include prokaryotic and eukaryotic cells, organs, tissues or organisms, such as bacteria, yeast, fungus, and plant tissue.

“Progeny” of a particular plant, as used herein, refers to any descendents of the plant containing all or part of the plant's DNA.

“Promoter”, as used herein, refers to the 5′-flanking, non-coding sequence adjacent a coding sequence that is involved in the initiation of transcription of the coding sequence.

“Protoplast”, as used herein, refers to an isolated plant cell without cell walls, having the potency for regeneration into cell culture or a whole plant.

“Purified”, as used herein, when referring to a peptide or nucleotide sequence, indicates that the molecule is present in the substantial absence of other biological macromolecular, for example, polypeptides, polynucleic acids, and the like of the same type. The term “purified” as used herein preferably means at least 95% by weight, more preferably at least 99.8% by weight, of biological macromolecules of the same type present (but water, buffers, and other small molecules, especially molecules having a molecular weight of less than 1000 can be present).

“Pure”, as used herein, preferably has the same numerical limits as “purified” immediately above. “Substantially purified”, as used herein, refers to nucleic or amino acid sequences that are removed from their natural environment, isolated or separated, and are at least 60% free, preferably 75% free, and most preferably 90% free from other components with which they are naturally associated.

“Recombinant plant viral nucleic acid”, as used herein, refers to a plant viral nucleic acid that has been modified to contain non-native nucleic acid sequences. These non-native nucleic acid sequences may be from any organism or purely synthetic, however, they may also include nucleic acid sequences naturally occurring in the organism into which the recombinant plant viral nucleic acid is to be introduced.

“Recombinant plant virus”, as used herein, refers to a plant virus containing a recombinant plant viral nucleic acid.

“Regulatory region” or “regulatory sequence”, as used herein, in reference to a specific gene refers to the non-coding nucleotide sequences within that gene that are necessary or sufficient to provide for the regulated expression of the coding region of a gene. Thus the term regulatory region includes promoter sequences, regulatory protein binding sites, upstream activator sequences, and the like. Specific nucleotides within a regulatory region may serve multiple functions. For example, a specific nucleotide may be part of a promoter and participate in the binding of a transcriptional activator protein.

“Replication origin”, as used herein, refers to the minimal terminal sequences in linear viruses that are necessary for viral replication.

“Replicon”, as used herein, refers to an arrangement of RNA sequences generated by transcription of a transgene that is integrated into the host DNA that is capable of replication in the presence of a helper virus. A replicon may require sequences in addition to the replication origins for efficient replication and stability.

“Sample”, as used herein, is used in its broadest sense. A biological sample suspected of containing nucleic acid encoding a polypeptide (for example, a polypeptide encoded by a nucleic acid of the present invention) or fragments thereof may comprise a tissue, a cell, an extract from cells, chromosomes isolated from a cell (for example, a spread of metaphase chromosomes), genomic DNA (in solution or bound to a solid support such as for Southern analysis), RNA (in solution or bound to a solid support such as for northern analysis), cDNA (in solution or bound to a solid support), and the like.

“Silent mutation”, as used herein, refers to a mutation that has no apparent effect on the phenotype of the organism.

“Site-directed mutagenesis”, as used herein, refers to the in vitro induction of mutagenesis at a specific site in a given target nucleic acid molecule.

“Subgenomic promoter”, as used herein, refers to a promoter of a subgenomic mRNA of a viral nucleic acid.

“Specific binding” or “specifically binding”, as used herein, in reference to the interaction of an antibody and a protein or peptide, mean that the interaction is dependent upon the presence of a particular structure (the antigenic determinant or epitope) on the protein; in other words, the antibody is recognizing and binding to a specific protein structure rather than to proteins in general.

“Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See for example, Anderson and Young, Quantitative Filter Hybridization, in Nucleic Acid Hybridization [1985]). Other references include more sophisticated computations that take structural as well as sequence characteristics into account for the calculation of Tₘ.

“Stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (for example, hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (for example, hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

“Substitution”, as used herein, refers to a change made in an amino acid of nucleotide sequence that results in the replacement of one or more amino acids or nucleotides by different amino acids or nucleotides, respectively.

“Symptom”, as used herein refers to a visual condition resulting from the action of a vector or a clone insert of the present invention.

“Systemic infection”, as used herein, denotes infection throughout a substantial part of an organism including mechanisms of spread other than mere direct cell inoculation but rather including transport from one infected cell to additional cells either nearby or distant.

“Transcription”, as used herein, refers to the production of an RNA molecule by RNA polymerase as a complementary copy of a DNA sequence.

“Transcription termination region”, as used herein, refers to the sequence that controls formation of the 3′ end of the transcript. Self-cleaving ribozymes and polyadenylation sequences are examples of transcription termination sequences.

“Transformation”, as used herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells that transiently express the inserted DNA or RNA for limited periods of time.

“Transfection”, as used herein, refers to the introduction of foreign nucleic acid into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transfection may, for example, result in cells in which the inserted nucleic acid is capable of replication either as an autonomously replicating molecule or as part of the host chromosome, or cells that transiently express the inserted nucleic acid for limited periods of time.

“Transposon”, as used herein, refers to a nucleotide sequence such as a DNA or RNA sequence that is capable of transferring location or moving within a gene, a chromosome or a genome.

“Transgenic plant”, as used herein, refers to a plant that contains a foreign nucleotide sequence inserted into either its nuclear genome or organellar genome.

“Transgene”, as used herein, refers to a nucleic acid sequence that is inserted into a host cell or host cells by a transformation technique.

“Variants” of a polypeptide (for example, a polypeptide encoded by a nucleic acid of the present invention), as used herein, refers to a sequence resulting when a polypeptide is altered by one or more amino acids. The variant may have “conservative” changes, wherein a substituted amino acid has similar structural or chemical properties, for example, replacement of leucine with isoleucine. More rarely, a variant may have “nonconservative” changes, for example, replacement of a glycine with a tryptophan. Variants may also include sequences with amino acid deletions or insertions, or both. Guidance in determining which amino acid residues may be substituted, inserted, or deleted without abolishing biological or immunological activity may be found using computer programs well known in the art.

“Vector”, as used herein, refers to a DNA and/or RNA molecule, typically a plasmid containing an origin of replication, that transfers a nucleic acid segment between cells.

“Virion”, as used herein, refers to a particle composed of viral RNA and viral capsid protein.

“Virus”, as used herein, refers to an infectious agent composed of a nucleic acid encapsidated in a protein. A virus may be a mono-, di-, tri- or multi-partite virus.

### DESCRIPTION OF THE INVENTION

### I. Identification of Nucleotide and Amino Acid Sequences

The invention is based on the discovery of deoxyribonucleic acid (DNA) and amino acid sequences that confer an altered visual phenotype when expressed in plants. In particular, the present invention encompasses the nucleic acid sequences encoded by SEQ ID NOs:1-2065 and variants and portions thereof. In preferred embodiments, the sequences produce an altered visual phenotype when expressed in a plant. Examples of altered visual phenotypes include, but are not limited to chlorotic, bleaching, etching, wilting, necrosis, auxin response, dark green, gray leaf, wet leaf, fluorescent, and texture phenotypes and combinations thereof These sequences are contiguous sequences prepared from a database of 5′ single pass sequences and are thus referred to as contig sequences.

Nucleic acids of the present invention were identified in clones generated from a variety of cDNA libraries. The cDNA libraries were constructed in the GENEWARE® vector. The GENEWARE® vector is described in U.S. application Ser. No. 09/008,186 (incorporated herein by reference). Each of the complete set of clones from the GENEWARE® library were used to prepare an infectious viral unit. An infectious unit corresponding to each clone was used to inoculate Nicotiana benthamiana (a dicotyledonous plant). The plants were grown under identical conditions and a phenotypic analysis of each plant was carried out. The altered visual phenotype was observed in the plants that had been infected by an infectious unit created from the nucleic acids of the present invention.

Accordingly, this present invention encompasses the discovery of genes which, when introduced into plants, result in a reproducible phenotype. These phenotypes include, but are not limited to, stunting, chlorosis, bleaching, etching, wilting, necrosis, stem curling, an auxin response, chlorotic etching, elongation, wet leaf, gray leaf, dark green color, fluorescent, and changes in leaf surface features. It is contemplated that the functions of the various genes suggested by the observation of these phenotypes, either singly or in combination, can lead to the utilization of these genes for development and implementation of agronomic traits that are beneficial to the farmer. Examples of these utilities are described in the following list.

(1) Genes that are demonstrated to affect growth regulation of the plant (stunting, elongation, etc) could be utilized in the following areas of agriculture and/or horticulture:

a) Creation of dwarf varieties of any plant species.

b) Creation of plants that have controlled meristematic growth such that a desired plant height or plant form is achieved.

c) Creation of plants that have increased stem strength.

d) Creation of plants that have increased stem thickness.

e) Creation of plants that have increased lateral root proliferation

f) Creation of plants that have a lengthened vegetative phase of plant development to achieve increased plant mass and yield.

g) Creation of plants that have a shortened vegetative phase of plant development to achieve yields in a short growing season.

h) Creation of plants that undergo senescence or programmed death at a desired time

(2) Genes that are demonstrated to lead to altered leaf surfaces in plants could be utilized in the following areas of agriculture and/or horticulture:

a) Creation of plant varieties that have resistance to insects, fungi, bacteria and other plant pests.

b) Creation of plant varieties that have resistance to heat stress, cold stress, drought stress and other abiotic stresses on plants.

c) Creation of plants that have an increased uptake of agrichemicals that are applied to the plant for protection against plant pests.

d) Creation of plants that have an increase in the production of lipids used as products in agribusiness.

(3) Genes that are demonstrated to lead to changes in pigment content in plants could be utilized in the following areas of agriculture and/or horticulture:

a) Creation of plants that undergo senescence or programmed death at a desired time.

b) Creation of plants that have an increase in the production of lipids used as products in agribusiness.

c) Creation of plants that have senescence delayed to a specific time in the growing season.

d) Creation of plants that have increased tolerance to sub-optimal levels of macro and micro nutrients in the soil.

e) Creation of plants that have the desired levels of leaf pigments.

f) Creation of plants that have enhanced export of nutrients out of the leaves during seed filling.

g) Creation of plants that have enhanced import of nutrients into the leaves during the plant vegetative phase.

h) Creation of plants that have enhanced production of vitamins.

i) Creation of plants that undergo fruit ripening at a desired time.

j) Identification of herbicide target genes.

k) Identification of genes that confer resistance to herbicides.

(4) Genes that are demonstrated to lead to cell death or necrosis in plants could be utilized in the following areas of agriculture and/or horticulture:

a) Identification of herbicide target genes.

b) Creation of plants that have a controlled hypersensitive response to pathogen attack.

c) Creation of plants that have controlled induced systemic acquired resistance.

d) Creation of plants that have enhanced movement of water and solutes across membranes.

e) Creation of plants that have controlled production of ethylene.

f) Creation of plants that undergo senescence or programmed death at a desired time.

g) Creation of plants that have enhanced or controlled movement of peptides.

(5) Genes that are demonstrated to lead to etching in plants could be utilized in the following areas of agriculture and/or horticulture.

a) Creation of plants that have cell membranes that have resistance to heat stress, cold stress, drought stress, salt stress, heavy metal stress and other abiotic stresses.

b) Creation of plants that have cell membranes with enhanced transport of micro and macro nutrients.

c) Creation of plants that have cell membranes with enhanced transport of water and solutes.

d) Creation of plants that have cell membranes that have resistance to plant pathogens.

Additionally, the genes described herein may be used to enable the described utilities by introduction into a plant via the various methods developed for various crop plants. This could include introduction by the use of Agrobacterium tumefaciens, by microparticle bombardment, by whiskers, protoplast transformation or any other method commonly used for introduction of genes into plant tissues. Various promoters and regulatory elements can also be used to achieve the desired level of expression of the gene. The gene may be introduced into the plant to achieve ectopic expression at levels required to get the necessary effect. The gene may also be expressed in a sense or antisense configuration to achieve partial or complete down-regulation of the gene in the plant. When this is achieved using a sense expression the mechanism is believed to be via co-suppression or some other method of gene silencing. These embodiments are described in more detail below.

Following the identification of the altered visual phenotype in plant samples, further analyses of the sequences were carried out. In particular, the nucleotide sequences of the present invention were analyzed using bioinformatics methods as described below.

### II. Bioinformatics Methods

A. Phred, Phrap and Consed

Phred, Phrap and Consed are a set of programs that read DNA sequencer traces, make base calls, assemble the shotgun DNA sequence data and analyze the sequence regions that are likely to contribute to errors. Phred is the initial program used to read the sequencer trace data, call the bases and assign quality values to the bases. Phred uses a Fourier-based method to examine the base traces generated by the sequencer. The output files from Phred are written in FASTA, phd or scf format. Phrap is used to assemble contiguous sequences from only the highest quality portion of the sequence data output by Phred. Phrap is amenable to high-throughput data collection. Finally, Consed is used as a finishing tool to assign error probabilities to the sequence data. Detailed description of the Phred, Phrap and Consed software and its use can be found in the following references: Ewing et al., Genome Res., 8:175 [1998]; Ewing and Green, Genome Res. 8:186 [1998]; Gordon et al., Genome Res. 8: 195 [1998].

B. BLAST

The BLAST (Basic Local Alignment Search Tool) set of programs may be used to compare the large numbers of sequences and obtain homologies to known protein families. These homologies provide information regarding the function of newly sequenced genes. Detailed descriptions of the BLAST software and its uses can be found in the following references Altschul et al., J. Mol. Biol., 215:403 [1990]; Altschul, J. Mol. Biol. 219:555 [1991].

Generally, BLAST performs sequence similarity searching and is divided into 5 basic subroutines: (1) BLASTP compares an amino acid sequence to a protein sequence database; (2) BLASTN compares a nucleotide sequence to a nucleic acid sequence database; (3) BLASTX compares translated protein sequences done in 6 frames to a protein sequence database; (4) TBLASTN compares a protein sequence to a nucleotide sequence database that is translated into all 6 reading frames; (5) TBLASTX compares the 6 frame translated protein sequence to the 6-frame translation of a nucleotide sequence database. Subroutines (3)-(5) may be used to identify weak similarities in nucleic acid sequence.

The BLAST program is based on the High Segment Pair (HSP), two sequence fragments of arbitrary but equal length whose alignment is locally maximized and whose alignment meets or exceeds a cutoff threshold. BLAST determines multiple HSP sets statistically using sum statistics. The score of the HSP is then related to its expected chance of frequency of occurrence, E. The value, E, is dependent on several factors such as the scoring system, residue composition of sequences, length of query sequence and total length of database. In the output file will be listed these E values, typically in a histogram format, which are useful in determining levels of statistical significance at the user s predefined expectation threshold. Finally, the Smallest Sum Probability, P(N), is the probability of observing the shown matched sequences by chance alone and is typically in the range of 0-1.

BLAST measures sequence similarity using a matrix of similarity scores for all possible pairs of residues and these specify scores for aligning pairs of amino acids. The matrix of choice for a specific use depends on several factors: the length of the query sequence and whether or not a close or distant relationship between sequences is suspected. Several matrices are available including PAM40, PAM120, PAM250, BLOSUM 62 and BLOSUM 50. Altschul et al. (1990) found PAM120 to be the most broadly sensitive matrix (for example point accepted mutation matrix per 100 residues). However, in some cases the PAM120 matrix may not find short but strong or long but weak similarities between sequences. In these cases, pairs of PAM matrices may be used, such as PAM40 and PAM 250, and the results compared. Typically, PAM 40 is used for database searching with a query of 9-21 residues long, while PAM 250 is used for lengths of 47-123.

The BLOSUM (Blocks Substitution Matrix) series of matrices are constructed based on percent identity between two sequence segments of interest. Thus, the BLOSUM62 matrix is based on a matrix of sequence segments in which the members are less than 62% identical. BLOSUM62 shows very good performance for BLAST searching. However, other BLOSUM matrices, like the PAM matrices, may be useful in other applications. For example, BLOSUM45 is particularly strong in profile searching.

C. FASTA

The FASTA suite of programs permits the evaluation of DNA and protein similarity based on local sequence alignment. The FASTA search algorithm utilizes Smith/Waterma- and Needleman/Wunsch-based optimization methods. These algorithms consider all of the alignment possibilities between the query sequence and the library in the highest scoring sequence regions. The search algorithm proceeds in four basic steps:

1. The identities or pairs of identities between the two DNA or protein sequences are determined. The ktup parameter, as set by the user, is operative and determines how many consecutive sequence identities are required to indicate a match.

2. The regions identified in step I are re-scored using a PAM or BLOSUM matrix. This allows conservative replacements and runs of identities shorter than that specified by ktup to contribute to the similarity score.

3. The region with the single best scoring initial region is used to characterize pairwise similarity and these scores are used to rank the library sequences.

4. The highest scoring library sequences are aligned using the Smith-Waterman algorithm. This final comparison takes into account the possible alignments of the query and library sequence in the highest scoring region.

Further detailed description of the FASTA software and its use can be found in the following reference: Pearson and Lipman, Proc. Natl. Acad. Sci., 85: 2444 [1988].

D. Pfam

Despite the large number of different protein sequences determined through genomics-based approaches, relatively few structural and functional domains are known. Pfam is a computational method that utilizes a collection of multiple alignments and profile hidden Markov models of protein domain families to classify existing and newly found protein sequences into structural families. Detailed descriptions of the Pfam software and its uses can be found in the following references: Sonhammer et al., Proteins: Structure, Function and Genetics, 28:405 [1997]; Sonhammer et al., Nucleic Acids Res., 26:320 [1998]; Bateman et al., Nucleic Acids Res., 27: 260 [1999].

Pfam 3.1, the latest version, includes 54% of proteins in SWISS_PROT and SP-TrEMBL-5 as a match to the database and includes expectation values for matches. Pfam consists of parts A and B. Pfam-A contains a hidden Markov model and includes curated families. Pfam-B uses the Domainer program to cluster sequence segments not included in Pfam-A. Domainer uses pairwise homology data from Blastp to construct aligned families.

Alternative protein family databases that may be used include PRINTS and BLOCKS, which both are based on a set of ungapped blocks of aligned residues. However, these programs typically contain short conserved regions whereas Pfam represents a library of complete domains that facilitates automated annotation. Comparisons of Pfam profiles may also be performed using genomic and EST data with the programs, Genewise and ESTwise, respectively. Both of these programs allow for introns and frame shifting errors.

E. BLOCKS

The determination of sequence relationships between unknown sequences and those that have been categorized can be problematic because background noise increases with the number of sequences, especially at a low level of similarity detection. One recent approach to this problem has been tested that efficiently detects and confirms weak or distant relationships among protein sequences based on a database of blocks. The BLOCKS database provides multiple alignments of sequences and contains blocks or protein motifs found in known families of proteins.

Other programs such as PRINTS and Prodom also provide alignments, however, the BLOCKS database differs in the manner in which the database was constructed. Construction of the BLOCKS database proceeds as follows: one starts with a group of sequences that presumably have one or motifs in common, such as those from the PROSITE database. The PROTOMAT program then uses a motif finding program to scan sequences for similarity looking for spaced triplets of amino acids. The located blocks are then entered into the MOTOMAT program for block assembly. Weights are computed for all sequences. Following construction of a BLOCKS database one can use BLIMPS to performs searches of the BLOCKS database. Detailed description of the construction and use of a BLOCKS database can be found in the following references: Henikoff, S. and Henikoff, J. G., Genomics, 19:97 [1994]; Henikoff, J. G. and Henikoff, S., Meth. Enz., 266:88 [1996].

F. PRINTS

The PRINTS database of protein family fingerprints can be used in addition to BLOCKS and PROSITE. These databases are considered to be secondary databases because they diagnose the relationship between sequences that yield function information. Presently, however, it is not recommended that these databases be used alone. Rather, it is strongly suggested that these pattern databases be used in conjunction with each other so that a direct comparison of results can be made to analyze their robustness.

Generally, these programs utilize pattern recognition to discover motifs within protein sequences. However, PRINTS goes one step further, it takes into account not simply single motifs but several motifs simultaneously that might characterize a family signature. Other programs, such as PROSITE, rely on pattern recognition but are limited by the fact that query sequences must match them exactly. Thus, sequences that vary slightly will be missed. In contrast, the PRINTS database fingerprinting approach is capable of identifying distant relatives due to its reliance on the fact that sequences do not have match the query exactly. Instead they are scored according to how well they fit each motif in the signature. Another advantage of PRINTS is that it allows the user to search both PRINTS and PROSITE simultaneously. A detailed description of the use of PRINTS can be found in the following reference: Attwood et al., Nucleic Acids Res. 25: 212 [1997].

### III. Nucleic Acid Sequences, Including Related, Variant, Altered and Extended Sequences

This invention encompasses nucleic acids, polypeptides encoded by the nucleic acid sequences, and variants that retain at least one biological or other functional activity of the polynucleotide or polypeptide of interest. A preferred polynucleotide variant is one having at least 80%, and more preferably 90%, sequence identity to the sequence of interest. A most preferred polynucleotide variant is one having at least 95% sequence identity to the polynucleotide of interest.

In particularly preferred embodiments, the invention encompasses the polynucleotides comprising a polynucleotide encoded by SEQ ID NOs:1-2065. In particularly preferred embodiments, the nucleic acids are operably linked to an exogenous promoter (and in most preferred embodiments to a plant promoter) or present in a vector.

It will be appreciated by those skilled in the art that as a result of the degeneracy of the genetic code, a multitude of nucleotide sequences encoding a given polypeptide (for example, a polypeptide encoded by a nucleic acid of the present invention), some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequence of the naturally occurring polypeptide, and all such variations are to be considered as being specifically disclosed.

Although nucleotide sequences that encode a given polypeptide (for example, a polypeptide encoded by a nucleic acid of the present invention) and its variants are preferably capable of hybridizing to the nucleotide sequence of the naturally occurring polypeptide under appropriately selected conditions of stringency, it may be advantageous to produce nucleotide sequences encoding the polypeptide or its derivatives possessing a substantially different codon usage. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding a polypeptide and its derivatives without altering the encoded amino acid sequences include the production of RNA transcripts having more desirable properties, such as a greater half-life, than transcripts produced from the naturally occurring sequence.

The invention also encompasses production of DNA sequences, or portions thereof, that encode a polynucleotide and its variants, entirely by synthetic chemistry. After production, the synthetic sequence may be inserted into any of the many available expression vectors and cell systems using reagents that are well known in the art. Moreover, synthetic chemistry may be used to introduce mutations into a sequence encoding a polynucleotide of the present invention or any portion thereof.

Also encompassed by the invention are polynucleotide sequences that are capable of hybridizing to SEQ ID NOs:1-2065 under various conditions of stringency (for example, conditions ranging from low to high stringency). Hybridization conditions are based on the melting temperature (Tₘ) of the nucleic acid binding complex or probe, as taught in Wahl and Berger, Methods Enzymol., 152:399 [1987] and Kimmel, Methods Enzymol., 152:507 [1987], and may be used at a defined stringency.

Altered nucleic acid sequences encoding a polynucleotide of the present invention include deletions, insertions, or substitutions of different nucleotides resulting in a polynucleotide that encodes the same polypeptide or a functionally equivalent polynucleotide or polypeptide. The encoded protein may also contain deletions, insertions, or substitutions of amino acid residues that produce a silent change and result in a functionally equivalent polypeptide. Deliberate amino acid substitutions may be made on the basis of similarity in polarity, charge, solubility, hydrophobicity, hydrophilicity, and/or the amphipathic nature of the residues as long as the biological activity of the polypeptide is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; phenylalanine and tyrosine.

Also included within the scope of the present invention are alleles of the genes encoding polypeptides. As used herein, an “allele” or “allelic sequence” is an alternative form of the gene that may result from at least one mutation in the nucleic acid sequence. Alleles may result in altered mRNAs or polypeptides whose structure or function may or may not be altered. Any given gene may have none, one, or many allelic forms. Common mutational changes that give rise to alleles are generally ascribed to natural deletions, additions, or substitutions of nucleotides. Each of these types of changes may occur alone, or in combination with the others, one or more times in a given sequence.

Methods for DNA sequencing that are well known and generally available in the art may be used to practice any embodiments of the invention. The methods may employ such enzymes as the Klenow fragment of DNA polymerase I, SEQUENASE (US Biochemical Corporation, Cleveland, Ohio), TAQ polymerase (U.S. Biochemical Corporation, Cleveland, Ohio), thermostable T7 polymerase (Amersham Pharmacia Biotech, Chicago, Ill.), or combinations of recombinant polymerases and proofreading exonucleases such as the ELONGASE amplification system (Life Technologies, Rockville, Md.). Preferably, the process is automated with machines such as the MICROLAB 2200 (Hamilton Company, Reno, Nev.), PTC200 DNA Engine thermal cycler (MJ Research, Watertown, Mass.) and the ABI 377 DNA sequencer (Perkin Elmer).

The nucleic acid sequences encoding a polynucleotide of the present invention may be extended utilizing a partial nucleotide sequence and employing various methods known in the art to detect upstream sequences such as promoters and regulatory elements. For example, one method that may be employed, “restriction-site” PCR, uses universal primers to retrieve unknown sequence adjacent to a known locus (Sarkar, PCR Methods Applic. 2:318 [1993]). In particular, genomic DNA is first amplified in the presence of primer to linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region (Triglia et al., Nucleic Acids Res. 16:8186 [1988]). The primers may be designed using OLIGO 4.06 primer analysis software (National Biosciences Inc., Plymouth, Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method that may be used is capture PCR that involves PCR amplification of DNA fragments adjacent to a known sequence in human and yeast artificial chromosome DNA (Lagerstrom et al., PCR Methods Applic. 1:111 [1991]). In this method, multiple restriction enzyme digestions and ligations may also be used to place an engineered double-stranded sequence into an unknown portion of the DNA molecule before performing PCR.

Another method that may be used to retrieve unknown sequences is that of Parker et al., Nucleic Acids Res., 19:3055 [1991]. Additionally, one may use PCR, nested primers, and PROMOTERFINDER DNA Walking Kits libraries (Clontech, Palo Alto, Calif.) to walk in genomic DNA. This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into the 5′ and 3′ non-transcribed regulatory regions.

Capillary electrophoresis systems that are commercially available (for example, from PE Biosystems, Inc., Foster City, Calif.) may be used to analyze the size or confirm the nucleotide sequence of sequencing or PCR products. In particular, capillary sequencing may employ flowable polymers for electrophoretic separation, four different fluorescent dyes (one for each nucleotide) that are laser activated, and detection of the emitted wavelengths by a charge coupled device camera. Output/light intensity may be converted to electrical signal using appropriate software (for example, GENOTYPER and SEQUENCE NAVIGATOR from PE Biosystems, Foster City, Calif.) and the entire process from loading of samples to computer analysis and electronic data display may be computer controlled. Capillary electrophoresis is especially preferable for the sequencing of small pieces of DNA that might be present in limited amounts in a particular sample.

It is contemplated that the nucleic acids disclosed herein can be utilized as starting nucleic acids for directed evolution. In some embodiments, artificial evolution is performed by random mutagenesis (for example, by utilizing error-prone PCR to introduce random mutations into a given coding sequence). This method requires that the frequency of mutation be finely tuned. As a general rule, beneficial mutations are rare, while deleterious mutations are common. This is because the combination of a deleterious mutation and a beneficial mutation often results in an inactive enzyme. The ideal number of base substitutions for targeted gene is usually between 1.5 and 5 (Moore and Arnold, Nat. Biotech., 14, 458-67 [1996]; Leung et al., Technique, 1:11-15 [1989]; Eckert and Kunkel, PCR Methods Appl., 1:17-24 [1991]; Caldwell and Joyce, PCR Methods Appl., 2:28-33 (1992); and Zhao and Arnold, Nuc. Acids. Res., 25:1307-08 [1997]). After mutagenesis, the resulting clones are selected for desirable activity. Successive rounds of mutagenesis and selection are often necessary to develop enzymes with desirable properties. It should be noted that only the useful mutations are carried over to the next round of mutagenesis.

In other embodiments of the present invention, the polynucleotides of the present invention are used in gene shuffling or sexual PCR procedures (for example, Smith, Nature, 370:324-25 [1994]; U.S. Pat. Nos. 5,837,458; 5,830,721; 5,811,238; and 5,733,731, each of which is herein incorporated by reference). Gene shuffling involves random fragmentation of several mutant DNAs followed by their reassembly by PCR into full length molecules. Examples of various gene shuffling procedures include, but are not limited to, assembly following DNase treatment, the staggered extension process (STEP), and random priming in vitro recombination. In the DNase mediated method, DNA segments isolated from a pool of positive mutants are cleaved into random fragments with DNaseI and subjected to multiple rounds of PCR with no added primer. The lengths of random fragments approach that of the uncleaved segment as the PCR cycles proceed, resulting in mutations in present in different clones becoming mixed and accumulating in some of the resulting sequences. Multiple cycles of selection and shuffling have led to the functional enhancement of several enzymes (Stemmer, Nature, 370:398-91 [1994]; Stemmer, Proc. Natl. Acad. Sci. USA, 91, 10747-51 [1994]; Crameri et al., Nat. Biotech., 14:315-19 [1996]; Zhang et al., Proc. Natl. Acad. Sci. USA, 94:4504-09 [1997]; and Crameri et al., Nat. Biotech., 15:436-38 [1997]).

### IV. Vectors, Engineering, and Expression of Sequences

In another embodiment of the invention, the polynucleotide sequences of the present invention and fragments and portions thereof, may be used in recombinant DNA molecules to direct expression of an mRNA or polypeptide in appropriate host cells. Due to the inherent degeneracy of the genetic code, other DNA sequences that encode substantially the same or a functionally equivalent amino acid or mRNA sequence may be produced and these sequences may be used to clone and express polypeptides (for example, a polypeptide encoded by a nucleic acid of the present invention).

As will be understood by those of skill in the art, it may be advantageous to produce nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life that is longer than that of a transcript generated from the naturally occurring sequence.

The nucleotide sequences of the present invention can be engineered using methods generally known in the art in order to alter the polypeptide sequences for a variety of reasons, including but not limited to, alterations that modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences encoding a polypeptide may be ligated to a heterologous sequence to encode a fusion protein. For example, to screen peptide libraries for inhibitors of the polypeptides activity (for example, enzymatic activity), it may be useful to encode a chimeric protein that can be recognized by a commercially available antibody. A fusion protein may also be engineered to contain a cleavage site located between the polypeptide encoding sequence and the heterologous protein sequence, so that the polypeptide of interest may be cleaved and purified away from the heterologous moiety.

In another embodiment, sequences encoding a polypeptide (for example, a polypeptide encoded by a nucleic acid of the present invention) may be synthesized, in whole or in part, using chemical methods well known in the art (See for example, Caruthers et al., Nucl. Acids Res. Symp. Ser. 215 [1980]; Horn et al., Nucl. Acids Res. Symp. Ser. 225 [1980]). Alternatively, the protein itself may be produced using chemical methods to synthesize the amino acid sequence of the polypeptide of interest (for example, a polypeptide encoded by a nucleic acid of the present invention), or a portion thereof. For example, peptide synthesis can be performed using various solid-phase techniques (Roberge et al., Science 269:202 [1995]) and automated synthesis may be achieved, for example, using the ABI 431A peptide synthesizer (PE Corporation, Norwalk, Conn.).

The newly synthesized peptide may be substantially purified by preparative high performance liquid chromatography (See for example, Creighton, T. (1983) Proteins, Structures and Molecular Principles, WH Freeman and Co., New York, N.Y.). The composition of the synthetic peptides may be confirmed by amino acid analysis or sequencing (for example, the Edman degradation procedure; or Creighton, supra). Additionally, the amino acid sequence of the polypeptide of interest or any part thereof, may be altered during direct synthesis and/or combined using chemical methods with sequences from other proteins, or any part thereof, to produce a variant polypeptide.

In order to express a biologically active polypeptide (for example, a polypeptide encoded by a nucleic acid of the present invention) or RNA, the nucleotide sequences encoding the polypeptide or functional equivalents, may be inserted into appropriate expression vector, that is, a vector that contains the necessary elements for the transcription and translation of the inserted coding sequence.

Methods that are well known to those skilled in the art may be used to construct expression vectors containing sequences encoding polypeptides (for example, a polypeptide encoded by a nucleic acid of the present invention) and appropriate transcriptional and translational control elements. These methods include in vitro recombinant DNA techniques, synthetic techniques, and in vivo genetic recombination. Such techniques are described in Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

A variety of expression vector/host systems may be utilized to contain and express sequences encoding a polypeptide of interest. These include, but are not limited to, microorganisms such as bacteria transformed with recombinant bacteriophage, plasmid, or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV; brome mosaic virus) or with bacterial expression vectors (for example, Ti or pBR322 plasmids); or animal cell systems.

The “control elements” or “regulatory sequences” are those non-translated regions of the vector (for example, enhancers, promoters, 5′ and 3′ untranslated regions) that interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, LaJolla, Calif.) or PSPORT1 plasmid (Life Technologies, Inc., Rockville, Md.) and the like may be used. The baculovirus polyhedrin promoter may be used in insect cells. Promoters or enhancers derived from the genomes of plant cells (for example, heat shock, RUBISCO; and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence encoding a polypeptide, vectors based on SV40 or EBV may be used with an appropriate selectable marker.

In bacterial systems, a number of expression vectors may be selected depending upon the use intended for the polypeptide of interest. For example, when large quantities of the polypeptide are needed for the induction of antibodies, vectors that direct high level expression of fusion proteins that are readily purified may be used. Such vectors include, but are not limited to, the multifunctional E. coli cloning and expression vectors such as BLUESCRIPT phagemid (Stratagene, La Jolla, Calif.), in which the sequence encoding the polypeptide of interest may be ligated into the vector in frame with sequences for the amino-terminal Met and the subsequent 7 residues of beta-galactosidase so that a hybrid protein is produced; pIN vectors (Van Heeke and Schuster, J. Biol. Chem. 264:5503 [1989]; and the like. pGEMX vectors (Promega Corporation, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH may be used. For reviews, See for example, Ausubel et al. (supra) and Grant et al., Methods Enzymol. 153:516 [1987].

In cases where plant expression vectors are used, the expression of sequences encoding polypeptides may be driven by any of a number of promoters. In a preferred embodiment, plant vectors are created using a recombinant plant virus containing a recombinant plant viral nucleic acid, as described in PCT publication WO 96/40867. Subsequently, the recombinant plant viral nucleic acid that contains one or more non-native nucleic acid sequences may be transcribed or expressed in the infected tissues of the plant host and the product of the coding sequences may be recovered from the plant, as described in WO 99/36516.

An important feature of this embodiment is the use of recombinant plant viral nucleic acids that contain one or more non-native subgenomic promoters capable of transcribing or expressing adjacent nucleic acid sequences in the plant host and that result in replication and local and/or systemic spread in a compatible plant host. The recombinant plant viral nucleic acids have substantial sequence homology to plant viral nucleotide sequences and may be derived from an RNA, DNA, cDNA or a chemically synthesized RNA or DNA. A partial listing of suitable viruses is described below.

The first step in producing recombinant plant viral nucleic acids according to this particular embodiment is to modify the nucleotide sequences of the plant viral nucleotide sequence by known conventional techniques such that one or more non-native subgenomic promoters are inserted into the plant viral nucleic acid without destroying the biological function of the plant viral nucleic acid. The native coat protein coding sequence may be deleted in some embodiments, placed under the control of a non-native subgenomic promoter in other embodiments, or retained in a further embodiment. If it is deleted or otherwise inactivated, a non-native coat protein gene is inserted under control of one of the non-native subgenomic promoters, or optionally under control of the native coat protein gene subgenomic promoter. The non-native coat protein is capable of encapsidating the recombinant plant viral nucleic acid to produce a recombinant plant virus. Thus, the recombinant plant viral nucleic acid contains a coat protein coding sequence, which may be native or a nonnative coat protein coding sequence, under control of one of the native or non-native subgenomic promoters. The coat protein is involved in the systemic infection of the plant host.

Some of the viruses that meet this requirement include viruses from the tobamovirus group such as Tobacco Mosaic virus (TMV), Ribgrass Mosaic Virus (RGM), Cowpea Mosaic virus (CMV), Alfalfa Mosaic virus (AMV), Cucumber Green Mottle Mosaic virus watermelon strain (CGMMV-W) and Oat Mosaic virus (OMV) and viruses from the brome mosaic virus group such as Brome Mosaic virus (BMV), broad bean mottle virus and cowpea chlorotic mottle virus. Additional suitable viruses include Rice Necrosis virus (RNV), and geminiviruses such as tomato golden mosaic virus (TGMV), Cassava latent virus (CLV) and maize streak virus (MSV). However, the invention should not be construed as limited to using these particular viruses, but rather the method of the present invention is contemplated to include all plant viruses at a minimum.

Other embodiments of plant vectors used for the expression of sequences encoding polypeptides include, for example, viral promoters such as the 35S and 19S promoters of CaMV used alone or in combination with the omega leader sequence from TMV (Takamatsu, EMBO J. 6:307 [1987]). Alternatively, plant promoters such as the small subunit of RUBISCO or heat shock promoters may be used (Coruzzi et al., EMBO J. 3:1671 [1984]; Broglie et al., Science 224:838 [1984]; and Winter et al., Results Probl. Cell Differ. 17:85 [1991]). These constructs can be introduced into plant cells by direct DNA transformation or pathogen-mediated transfection. Such techniques are described in a number of generally available reviews (see for example, Hobbs, S. or Murry, L. E. in McGraw Hill Yearbook of Science and Technology (1992) McGraw Hill, New York, N.Y.; pp. 191-196.

The present invention further provides transgenic plants comprising the polynucleotides of the present invention. In some embodiments, the plant comprise more than one of the sequences. The sequences may be in the same vector or in different vectors. In some preferred embodiments, Agrobacterium mediated transfection is utilized to create transgenic plants. Since most dicotyledonous plant are natural hosts for Agrobacterium, almost every dicotyledonous plant may be transformed by Agrobacterium in vitro. Although monocotyledonous plants, and in particular, cereals and grasses, are not natural hosts to Agrobacterium, work to transform them using Agrobacterium has also been carried out (Hooykas-Van Slogteren et al. (1984) Nature 311:763-764). Plant genera that may be transformed by Agrobacterium include Arabidopsis, Chrysanthemum, Dianthus, Gerbera, Euphorbia, Pelaronium, Ipomoea, Passiflora, Cyclamen, Malus, Prunus, Rosa, Rubus, Populus, Santalum, Allium, Lilium, Narcissus, Ananas, Arachis, Phaseolus and Pisum.

For transformation with Agrobacterium, disarmed Agrobacterium cells are transformed with recombinant Ti plasmids of Agrobacterium tumefaciens or Ri plasmids of Agrobacterium rhizogenes (such as those described in U.S. Pat. No. 4,940,838, the entire contents of which are herein incorporated by reference). The nucleic acid sequence of interest is then stably integrated into the plant genome by infection with the transformed Agrobacterium strain. For example, heterologous nucleic acid sequences have been introduced into plant tissues using the natural DNA transfer system of Agrobacterium tumefaciens and Agrobacterium rhizogenes bacteria (for review, see Klee et al. (1987) Ann. Rev. Plant Phys. 38:467-486).

There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be transformed by Agrobacterium and (b) that the transformed cells or tissues can be induced to regenerate into whole plants. The third method is transformation of seeds, apices or meristems with Agrobacterium. This method requires micropropagation.

The efficiency of transformation by Agrobacterium may be enhanced by using a number of methods known in the art. For example, the inclusion of a natural wound response molecule such as acetosyringone (AS) to the Agrobacterium culture has been shown to enhance transformation efficiency with Agrobacterium tumefaciens (Shahla et al., (1987) Plant Molec. Biol. 8:291-298). Alternatively, transformation efficiency may be enhanced by wounding the target tissue to be transformed. Wounding of plant tissue may be achieved, for example, by punching, maceration, bombardment with microprojectiles, etc. (See e.g., Bidney et al., (1992) Plant Molec. Biol. 18:301-313).

In still further embodiments, the plant cells are transfected with vectors via particle bombardment (i.e., with a gene gun). Particle mediated gene transfer methods are known in the art, are commercially available, and include, but are not limited to, the gas driven gene delivery instrument descried in McCabe, U.S. Pat. No. 5,584,807, the entire contents of which are herein incorporated by reference. This method involves coating the nucleic acid sequence of interest onto heavy metal particles, and accelerating the coated particles under the pressure of compressed gas for delivery to the target tissue.

Other particle bombardment methods are also available for the introduction of heterologous nucleic acid sequences into plant cells. Generally, these methods involve depositing the nucleic acid sequence of interest upon the surface of small, dense particles of a material such as gold, platinum, or tungsten. The coated particles are themselves then coated onto either a rigid surface, such as a metal plate, or onto a carrier sheet made of a fragile material such as mylar. The coated sheet is then accelerated toward the target biological tissue. The use of the flat sheet generates a uniform spread of accelerated particles that maximizes the number of cells receiving particles under uniform conditions, resulting in the introduction of the nucleic acid sample into the target tissue.

An insect system may also be used to express polypeptides (for example, a polypeptide encoded by a nucleic acid of the present invention). For example, in one such system, Autographa californica nuclear polyhedrosis virus (AcNPV) is used as a vector to express foreign genes in Spodoptera frugiperda cells or in Trichoplusia larvae. The sequences encoding a polypeptide of interest may be cloned into a non-essential region of the virus, such as the polyhedrin gene, and placed under control of the polyhedrin promoter. Successful insertion of the nucleic acid sequence encoding the polypeptide of interest will render the polyhedrin gene inactive and produce recombinant virus lacking coat protein. The recombinant viruses may then be used to infect, for example, S. frugiperda cells or Trichoplusia larvae in which the polypeptide may be expressed (Engelhard et al., Proc. Nat. Acad. Sci. 91:3224 [1994]).

In mammalian host cells, a number of viral-based expression systems may be utilized. In cases where an adenovirus is used as an expression vector, sequences encoding polypeptides may be ligated into an adenovirus transcription/translation complex consisting of the late promoter and tripartite leader sequence. Insertion in a non-essential E1 or E3 region of the viral genome may be used to obtain a viable virus that is capable of expressing the polypeptide in infected host cells (Logan and Shenk, Proc. Natl. Acad. Sci., 81:3655 [1984]). In addition, transcription enhancers, such as the Rous sarcoma virus (RSV) enhancer, may be used to increase expression in mammalian host cells.

Specific initiation signals may also be used to achieve more efficient translation of sequences encoding the polypeptide of interest. Such signals include the ATG initiation codon and adjacent sequences. In cases where sequences encoding the polypeptide of interest, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers that are appropriate for the particular cell system that is used, such as those described in the literature (Scharf et al., Results Probl. Cell Differ., 20:125 [1994]).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing that cleaves a “prepro” form of the protein may also be used to facilitate correct insertion, folding and/or function. Different host cells such as CHO, HeLa, MDCK, HEK293, and WI38, that have specific cellular machinery and characteristic mechanisms for such post-translational activities, may be chosen to ensure the correct modification and processing of the foreign protein.

For long-term, high-yield production of recombinant proteins, stable expression is preferred. For example, cell lines that stably express the polypeptide of interest (for example, a polypeptide encoded by a nucleic acid of the present invention) may be transformed using expression vectors that may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 11:223 [1977]) and adenine phosphoribosyltransferase (Lowy et al., Cell 22:817 [1980]) genes that can be employed in tk⁻ or aprt⁻ cells, respectively. Also, antimetabolite, antibiotic, or herbicide resistance can be used as the basis for selection; for example, dhfr, which confers resistance to methotrexate (Wigler et al., Proc. Natl. Acad. Sci., 77:3567 [1980]); npt, which confers resistance to the aminoglycosides neomycin and G-418 (Colbere-Garapin et al., J. Mol. Biol., 150:1 [1981]); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman and Mulligan, Proc. Natl. Acad. Sci., 85:8047 [1988]). Recently, the use of visible markers has gained popularity with such markers as anthocyanins, α-glucuronidase and its substrate GUS, and luciferase and its substrate luciferin, being widely used not only to identify transformants, but also to quantify the amount of transient or stable protein expression attributable to a specific vector system (Rhodes et al., Methods Mol. Biol., 55:121 [1995]).

Although the presence/absence of marker gene expression suggests that the gene of interest is also present, its presence and expression may need to be confirmed. For example, if the sequence encoding a polypeptide is inserted within a marker gene sequence, recombinant cells containing sequences encoding the polypeptide can be identified by the absence of marker gene function. Alternatively, a marker gene can be placed in tandem with a sequence encoding the polypeptide under the control of a single promoter. Expression of the marker gene in response to induction or selection usually indicates expression of the tandem gene as well.

Alternatively, host cells that contain the nucleic acid sequence encoding the polypeptide of interest (for example, a polypeptide encoded by a nucleic acid of the present invention) and express the polypeptide may be identified by a variety of procedures known to those of skill in the art. These procedures include, but are not limited to, DNA-DNA or DNA-RNA hybridizations and protein bioassay or immunoassay techniques that include membrane, solution, or chip based technologies for the detection and/or quantification of nucleic acid or protein.

The presence of polynucleotide sequences encoding a polypeptide of interest (for example, a polypeptide encoded by a nucleic acid of the present invention) can be detected by DNA-DNA or DNA-RNA hybridization or amplification using probes or portions or fragments of polynucleotides encoding the polypeptide. Nucleic acid amplification based assays involve the use of oligonucleotides or oligomers based on the sequences encoding the polypeptide to detect transformants containing DNA or RNA encoding the polypeptide. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, that can be used as a probe or amplimer.

A variety of protocols for detecting and measuring the expression of a polypeptide (for example, a polypeptide encoded by a nucleic acid of the present invention), using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on the polypeptide is preferred, but a competitive binding assay may be employed. These and other assays are described, among other places, in Hampton et al., 1990; Serological Methods, a Laboratory Manual, APS Press, St Paul, Minn. and Maddox et al., J. Exp. Med., 158:1211 [1983]).

A wide variety of labels and conjugation techniques are known by those skilled in the art and may be used in various nucleic acid and amino acid assays. Means for producing labeled hybridization or PCR probes for detecting sequences related to polynucleotides encoding a polypeptide of interest include oligonucleotide labeling, nick translation, end-labeling or PCR amplification using a labeled nucleotide. Alternatively, the sequences encoding the polypeptide, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits from Pharmacia & Upjohn (Kalamazoo, Mich.), Promega Corporation (Madison, Wis.) and U.S. Biochemical Corp. (Cleveland, Ohio). Suitable reporter molecules or labels, which may be used, include radionuclides, enzymes, fluorescent, chemiluminescent, or chromogenic agents as well as substrates, cofactors, inhibitors, magnetic particles, and the like.

Host cells transformed with nucleotide sequences encoding a polypeptide of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides that encode the polypeptide of interest (for example, a polypeptide encoded by a nucleic acid of the present invention) may be designed to contain signal sequences that direct secretion of the polypeptide through a prokaryotic or eukaryotic cell membrane. Other recombinant constructions may be used to join sequences encoding the polypeptide to nucleotide sequence encoding a polypeptide domain that will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (available from Invitrogen, San Diego, Calif.) between the purification domain and the polypeptide of interest may be used to facilitate purification. One such expression vector provides for expression of a fusion protein containing the polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath et al., Prot. Exp. Purif., 3:263 [1992] while the enterokinase cleavage site provides a means for purifying the polypeptide from the fusion protein. A discussion of vectors that contain fusion proteins is provided in Kroll et al., DNA Cell Biol., 12:441 [1993]).

In addition to recombinant production, fragments of the polypeptide of interest may be produced by direct peptide synthesis using solid-phase techniques (Merrifield, J. Am. Chem. Soc., 85:2149 [1963]). Protein synthesis may be performed using manual techniques or by automation. Automated synthesis may be achieved, for example, using the Applied Biosystems 431A peptide synthesizer (Perkin Elmer). Various fragments of the polypeptide may be chemically synthesized separately and combined using chemical methods to produce the full length molecule.

### V. Alteration of Gene Expression

It is contemplated that the polynucleotides of the present invention (for example, SEQ ID NOs:1-311 and 2024-2065) may be utilized to either increase or decrease the level of corresponding mRNA and/or protein in transfected cells as compared to the levels in wild-type cells. Accordingly, in some embodiments, expression in plants by the methods described above leads to the overexpression of the polypeptide of interest in transgenic plants, plant tissues, or plant cells. The present invention is not limited to any particular mechanism. Indeed, an understanding of a mechanism is not required to practice the present invention. However, it is contemplated that overexpression of the polynucleotides of the present invention will alter the expression of the gene comprising the nucleic acid sequence of the present invention.

In other embodiments of the present invention, the polynucleotides are utilized to decrease the level of the protein or mRNA of interest in transgenic plants, plant tissues, or plant cells as compared to wild-type plants, plant tissues, or plant cells. One method of reducing protein expression utilizes expression of antisense transcripts (for example, U.S. Pat. Nos. 6,031,154; 5,453,566; 5,451,514; 5,859,342; and 4,801,340, each of which is incorporated herein by reference). Antisense RNA has been used to inhibit plant target genes in a tissue-specific manner (for example, Van der Krol et al., Biotechniques 6:958-976 [1988]). Antisense inhibition has been shown using the entire cDNA sequence as well as a partial cDNA sequence (for example, Sheehy et al., Proc. Natl. Acad. Sci. USA 85:8805-8809 [1988]; Cannon et al., Plant Mol. Biol. 15:39-47 [1990]). There is also evidence that 3′ non-coding sequence fragment and 5′ coding sequence fragments, containing as few as 41 base pairs of a 1.87 kb cDNA, can play important roles in antisense inhibition (Ch'ng et al., Proc. Natl. Acad. Sci. USA 86:10006-10010 [1989]).

Accordingly, in some embodiments, the nucleic acids of the present invention (for example, SEQ ID NOs: 1-311 and 2024-2065, and fragments and variants thereof) are oriented in a vector and expressed so as to produce antisense transcripts. To accomplish this, a nucleic acid segment from the desired gene is cloned and operably linked to a promoter such that the antisense strand of RNA will be transcribed. The expression cassette is then transformed into plants and the antisense strand of RNA is produced. The nucleic acid segment to be introduced generally will be substantially identical to at least a portion of the endogenous gene or genes to be repressed. The sequence, however, need not be perfectly identical to inhibit expression. The vectors of the present invention can be designed such that the inhibitory effect applies to other proteins within a family of genes exhibiting homology or substantial homology to the target gene.

Furthermore, for antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and up to about the full length of the coding region should be used, although a sequence of at least about 100 nucleotides is preferred, a sequence of at least about 200 nucleotides is more preferred, and a sequence of at least about 500 nucleotides is especially preferred.

Catalytic RNA molecules or ribozymes can also be used to inhibit expression of the target gene or genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs that are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, Solanum nodiflorum mottle virus and subterranean clover mottle virus. The design and use of target RNA-specific ribozymes is described in Haseloff, et al., Nature 334:585-591 (1988).

Another method of reducing protein expression utilizes the phenomenon of cosuppression or gene silencing (for example, U.S. Pat. Nos. 6,063,947; 5,686,649; and 5,283,184; each of which is incorporated herein by reference). The phenomenon of cosuppression has also been used to inhibit plant target genes in a tissue-specific manner. Cosuppression of an endogenous gene using a full-length cDNA sequence as well as a partial cDNA sequence (730 bp of a 1770 bp cDNA) are known (for example, Napoli et al., Plant Cell 2:279-289 [1990]; van der Krol et al., Plant Cell 2:291-299 [1990]; Smith et al., Mol. Gen. Genetics 224:477-481 [1990]). Accordingly, in some embodiments the nucleic acids (for example, SEQ ID NOs: 1-311 and 2024-2065, and fragments and variants thereof) from one species of plant are expressed in another species of plant to effect cosuppression of a homologous gene. Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 95% to absolute identity would be most preferred. As with antisense regulation, the effect should apply to any other proteins within a similar family of genes exhibiting homology or substantial homology.

For cosuppression, the introduced sequence in the expression cassette, needing less than absolute identity, also need not be full length, relative to either the primary transcription product or fully processed mRNA. This may be preferred to avoid concurrent production of some plants that are overexpressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges noted above for antisense regulation is used.

### VI. Expression of Sequences Producing Altered Visual Phenotypes

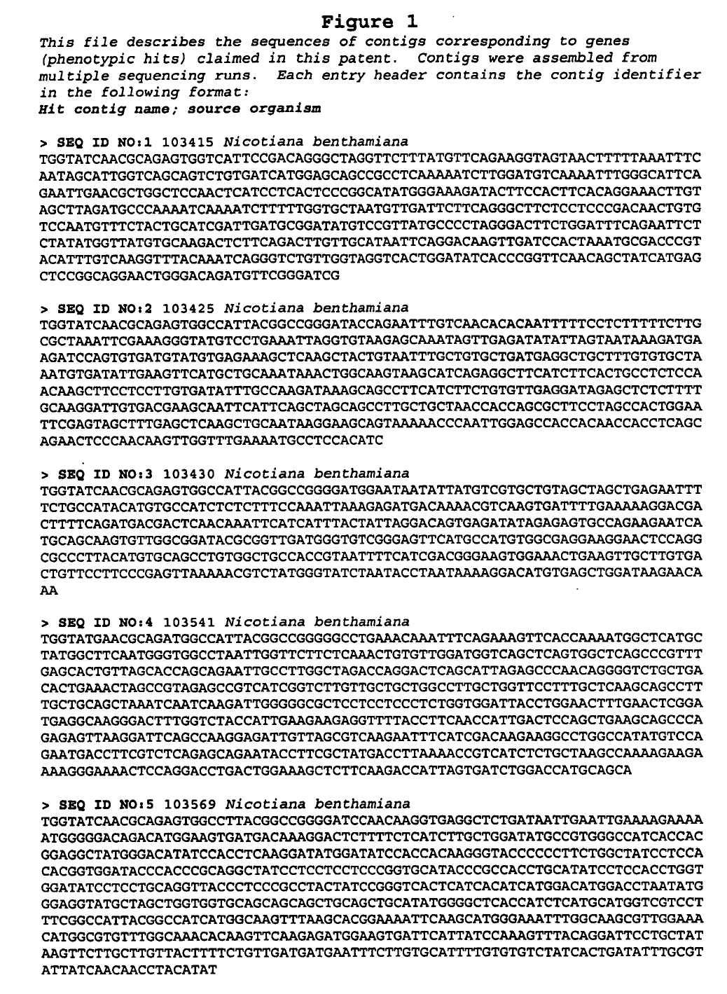

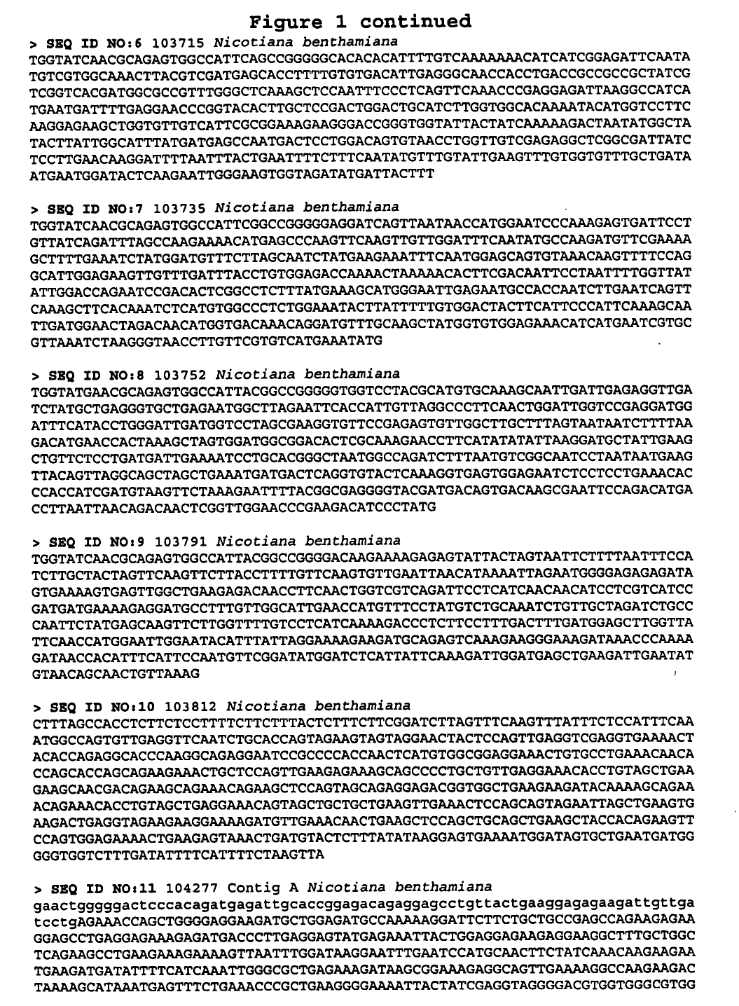

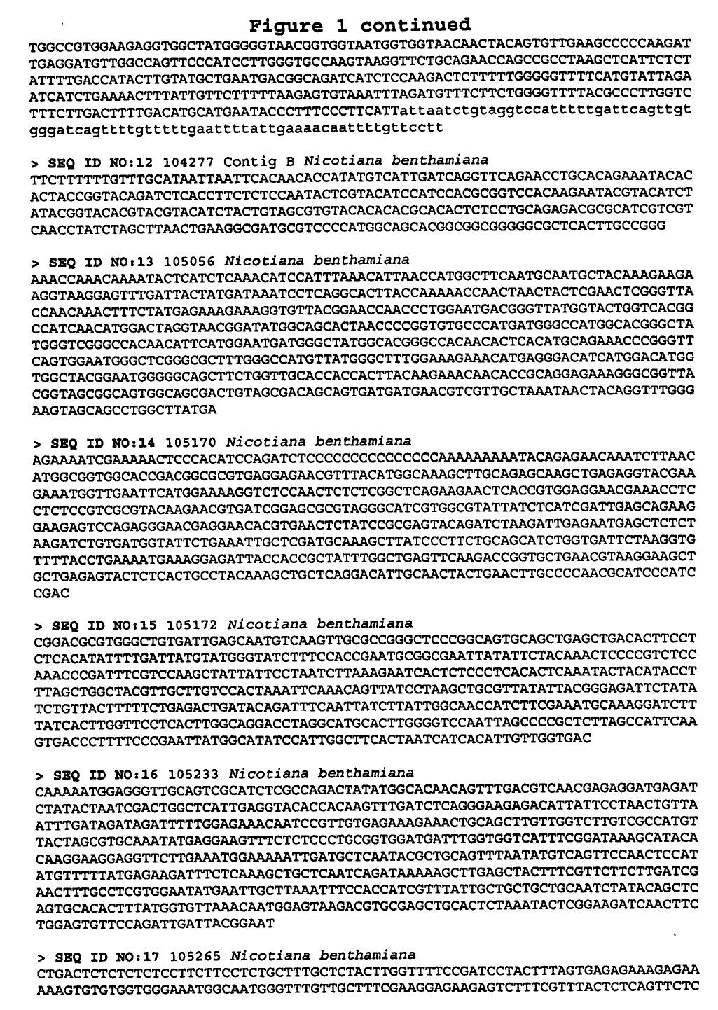

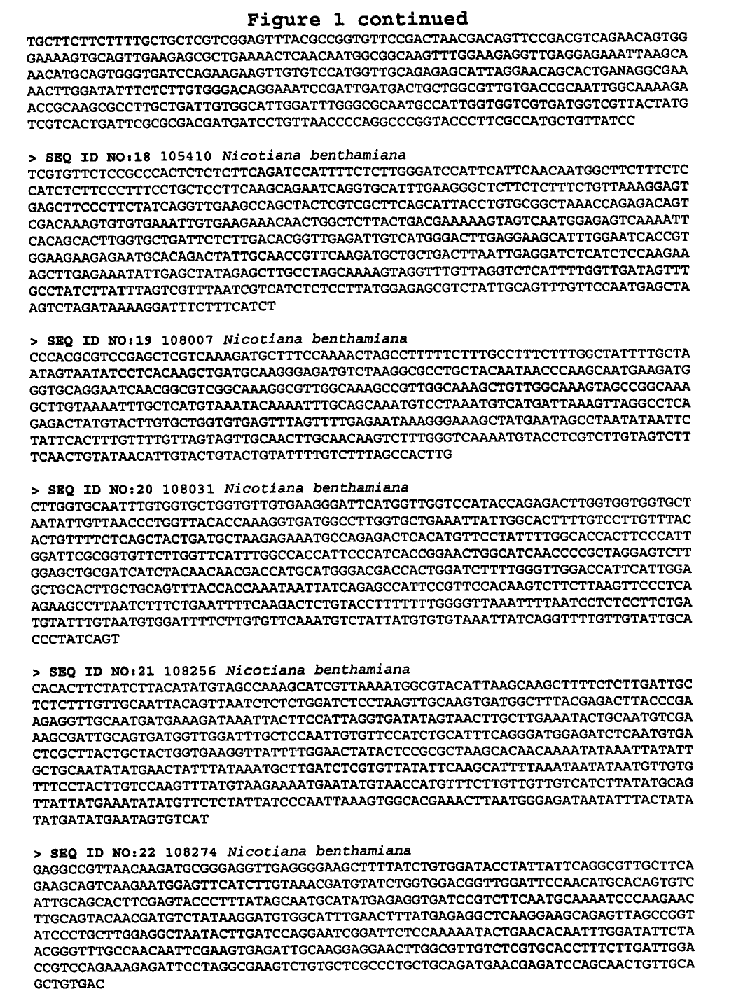

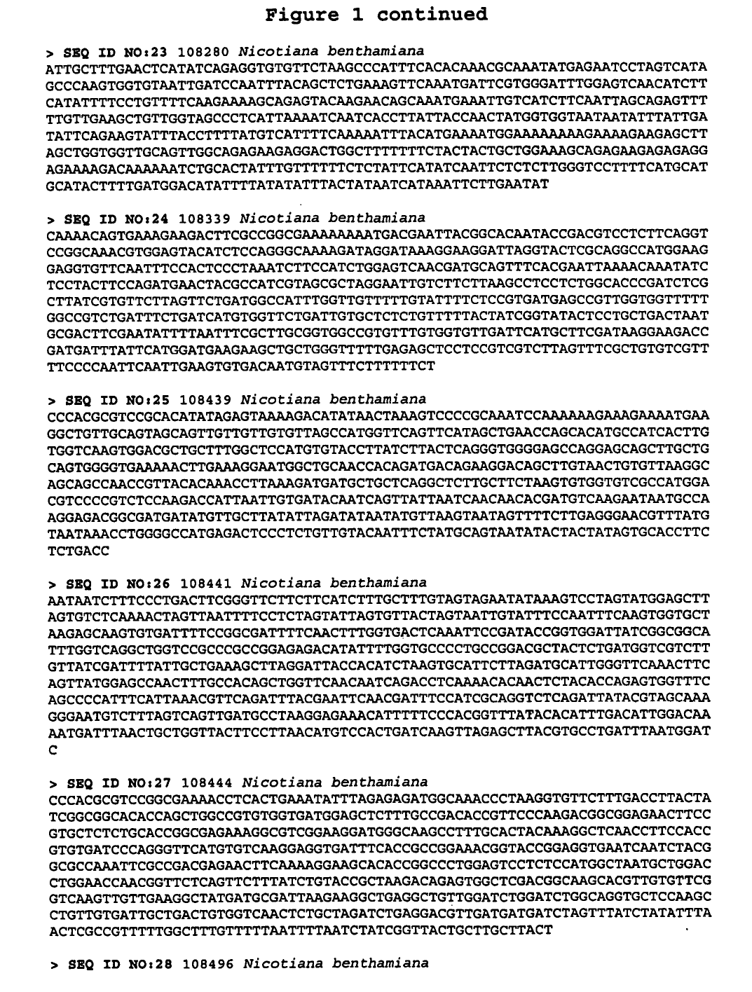

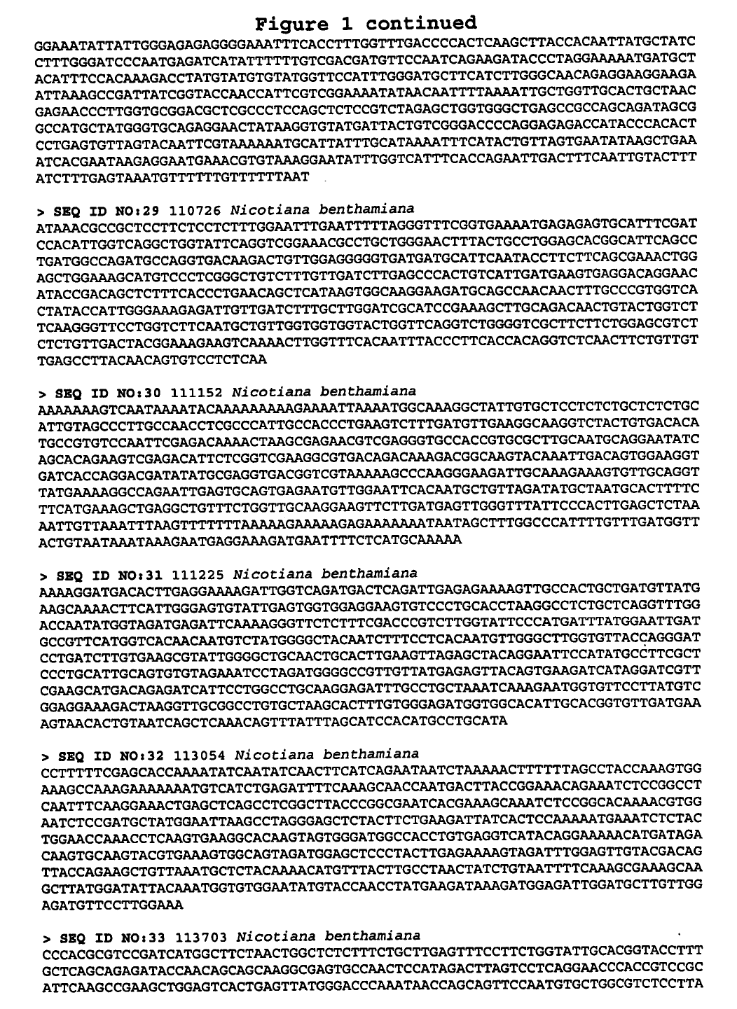

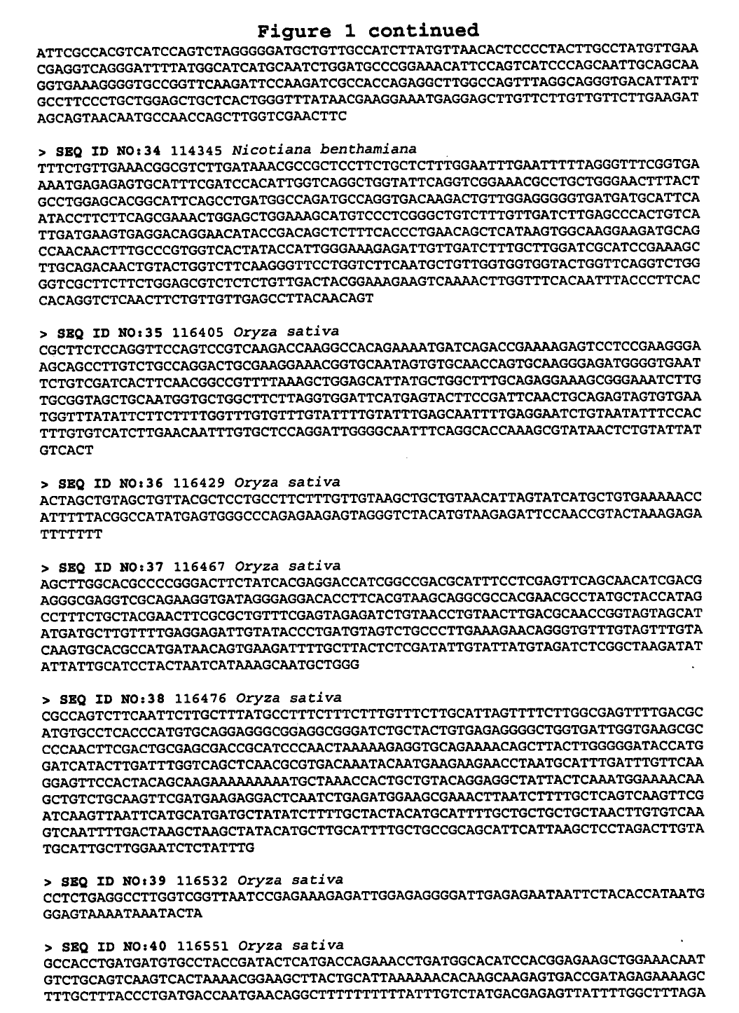

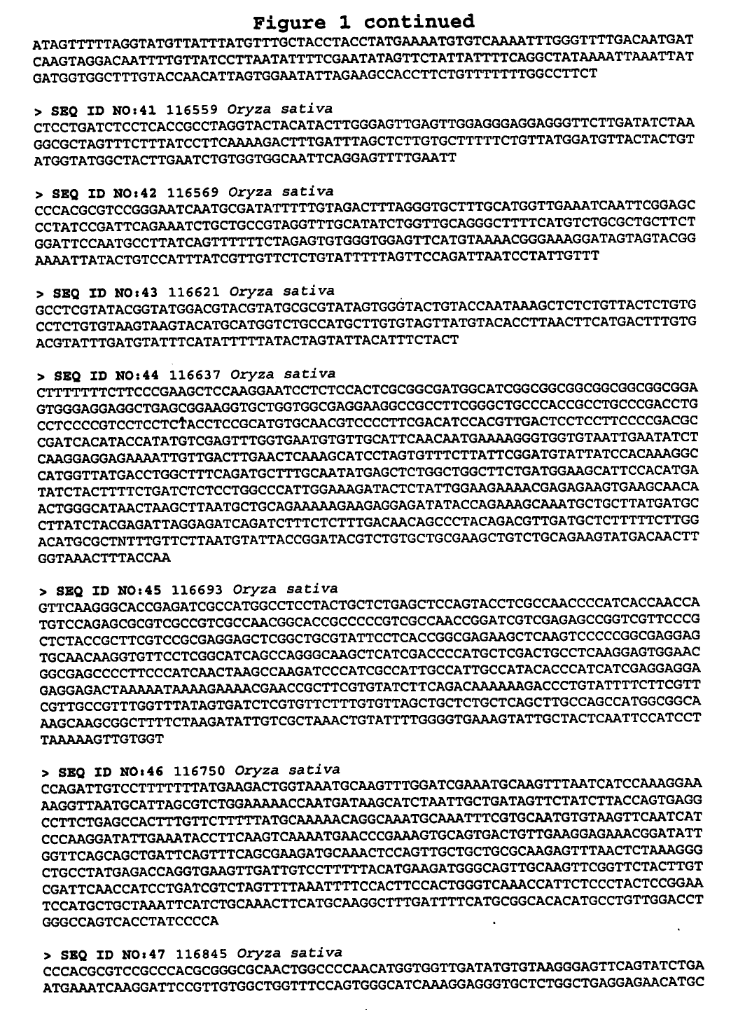

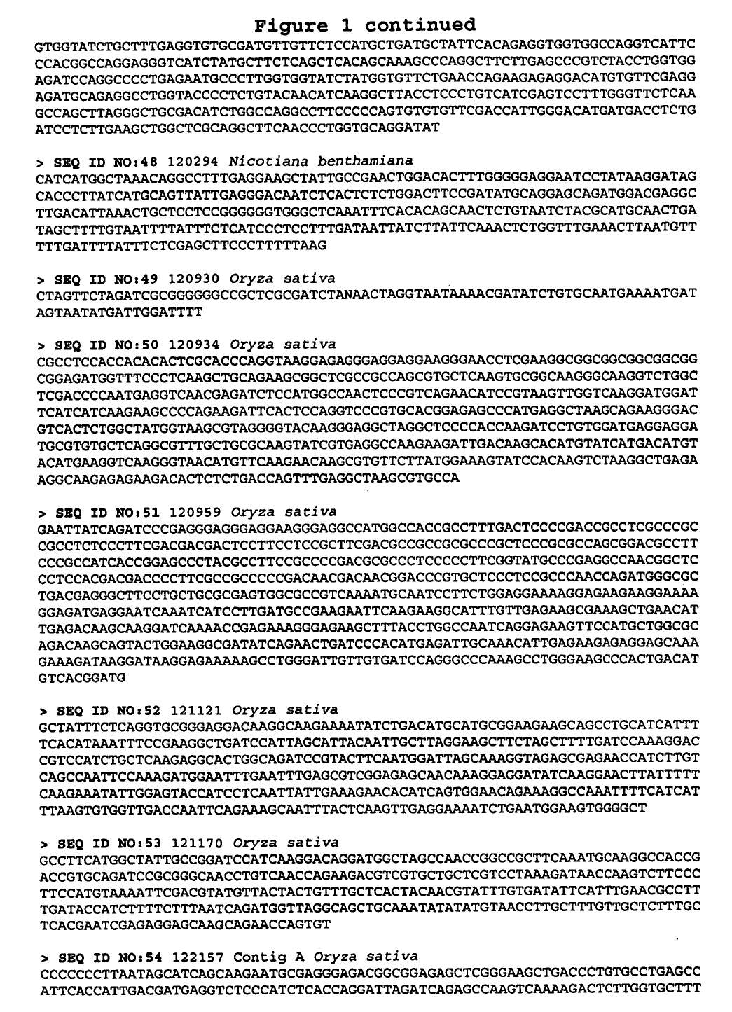

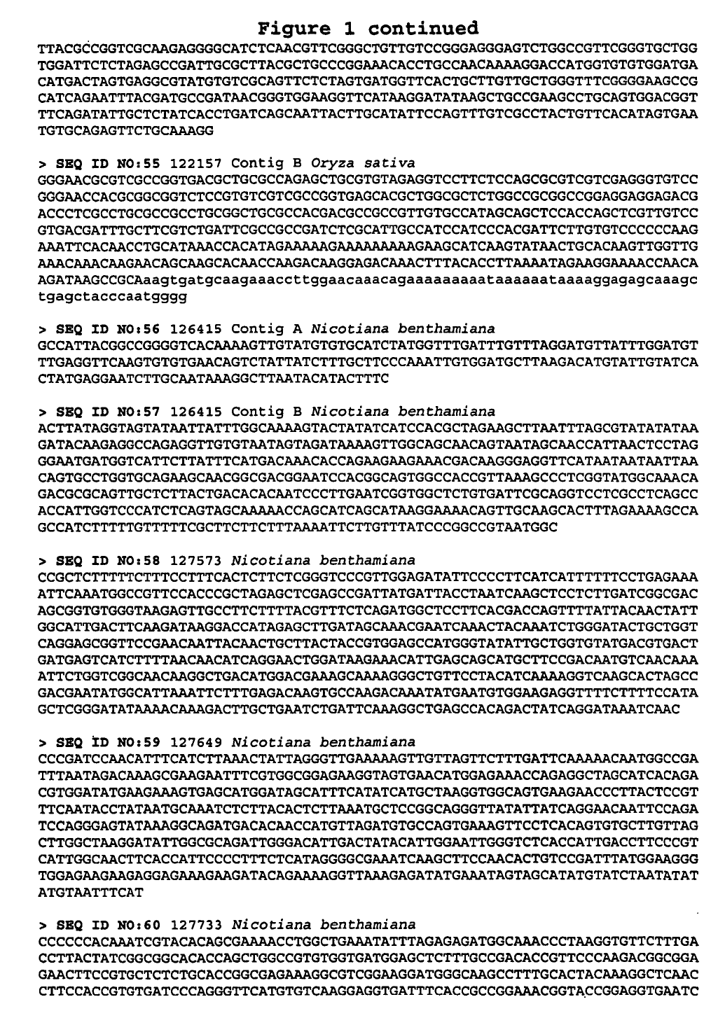

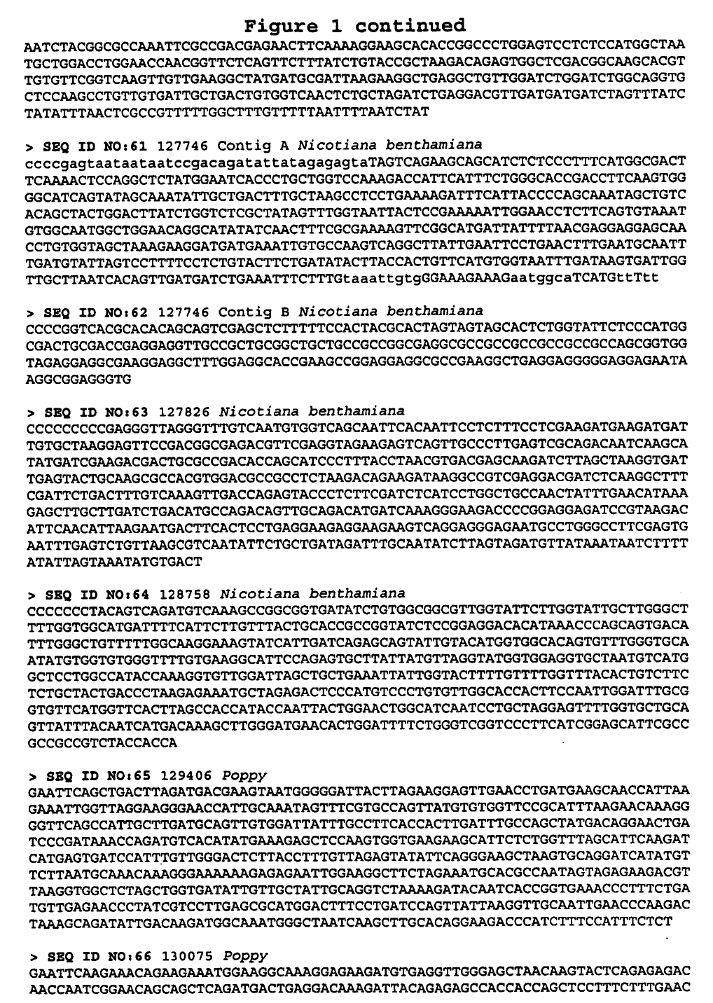

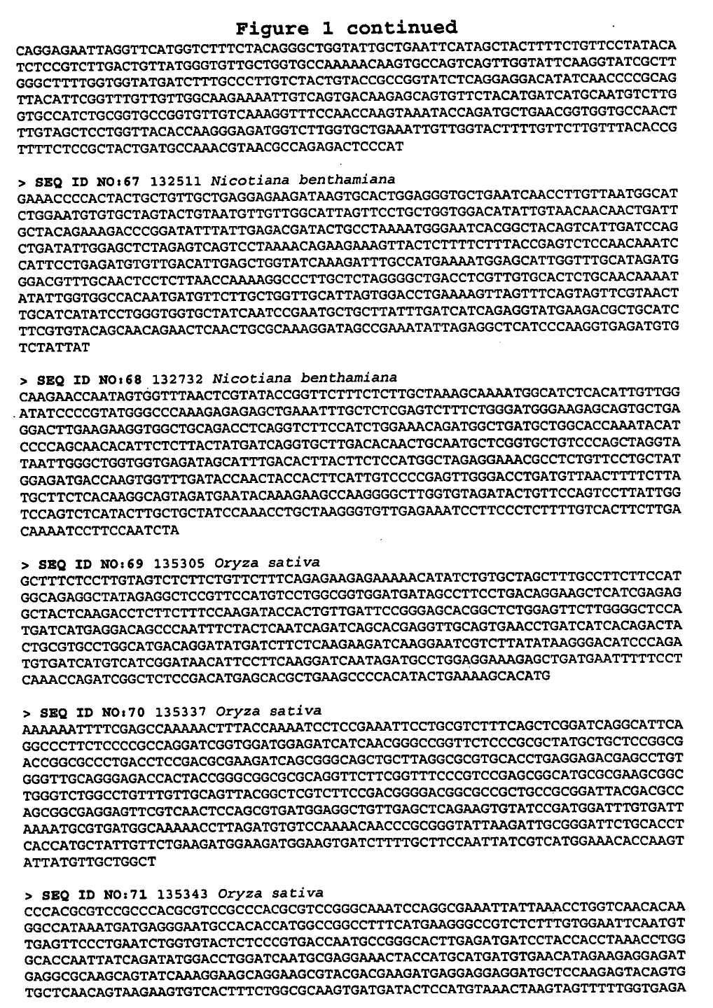

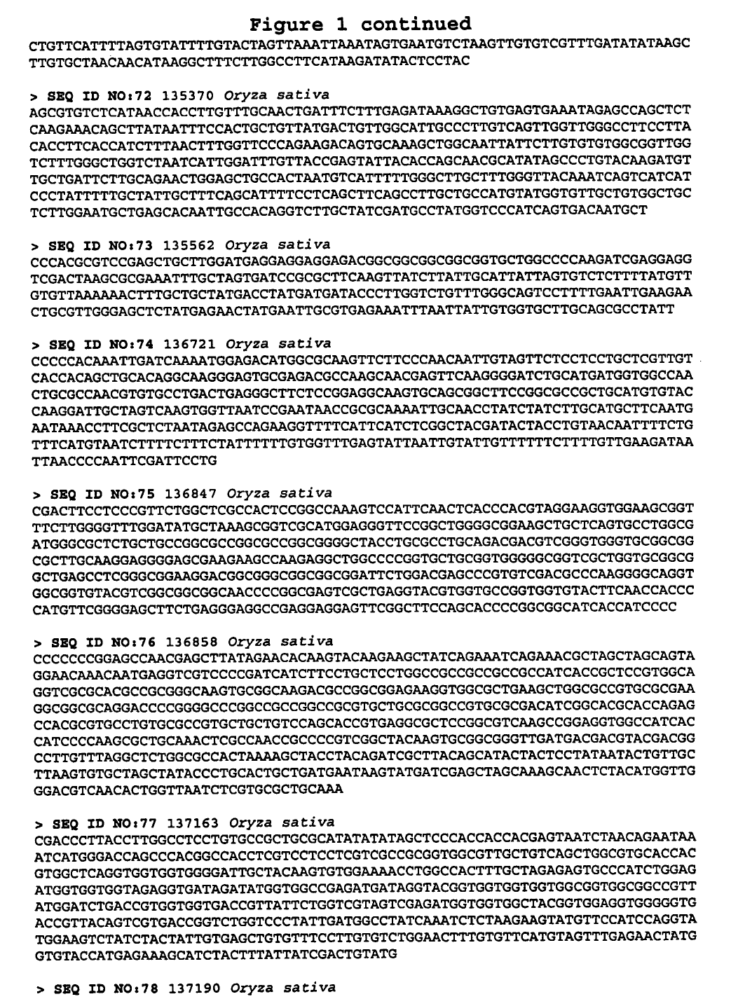

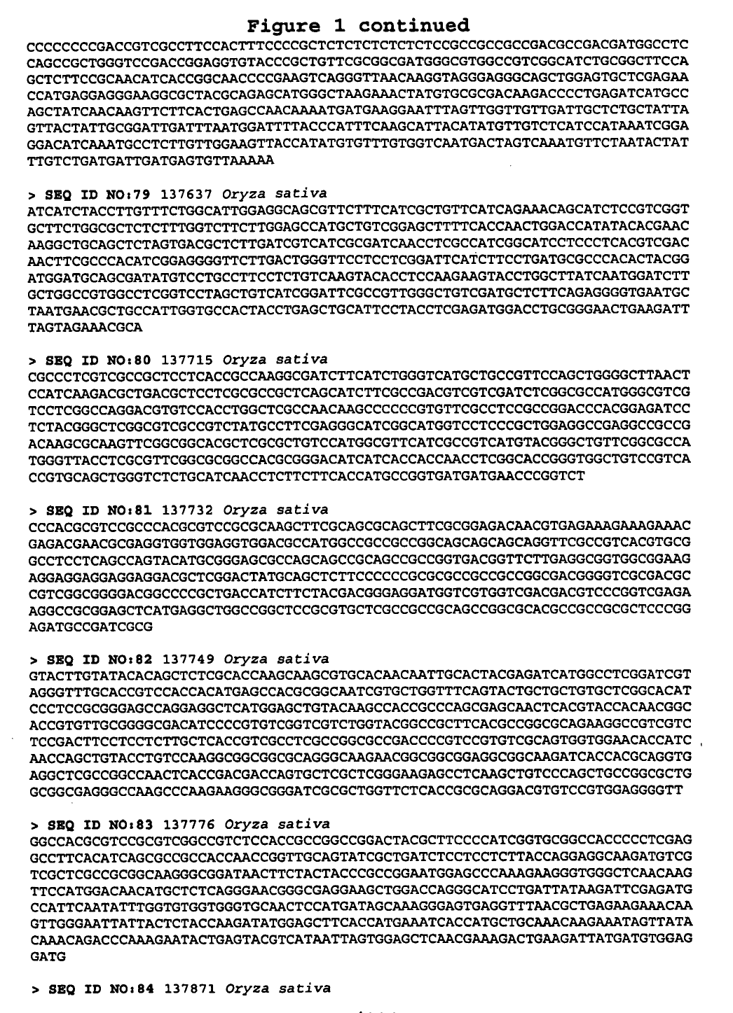

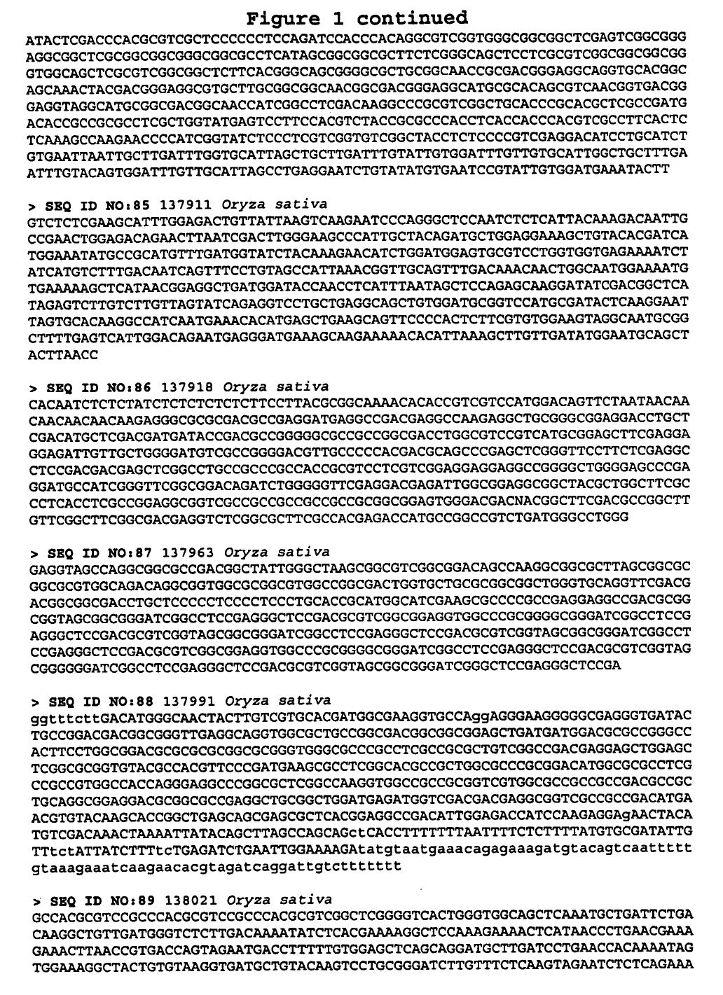

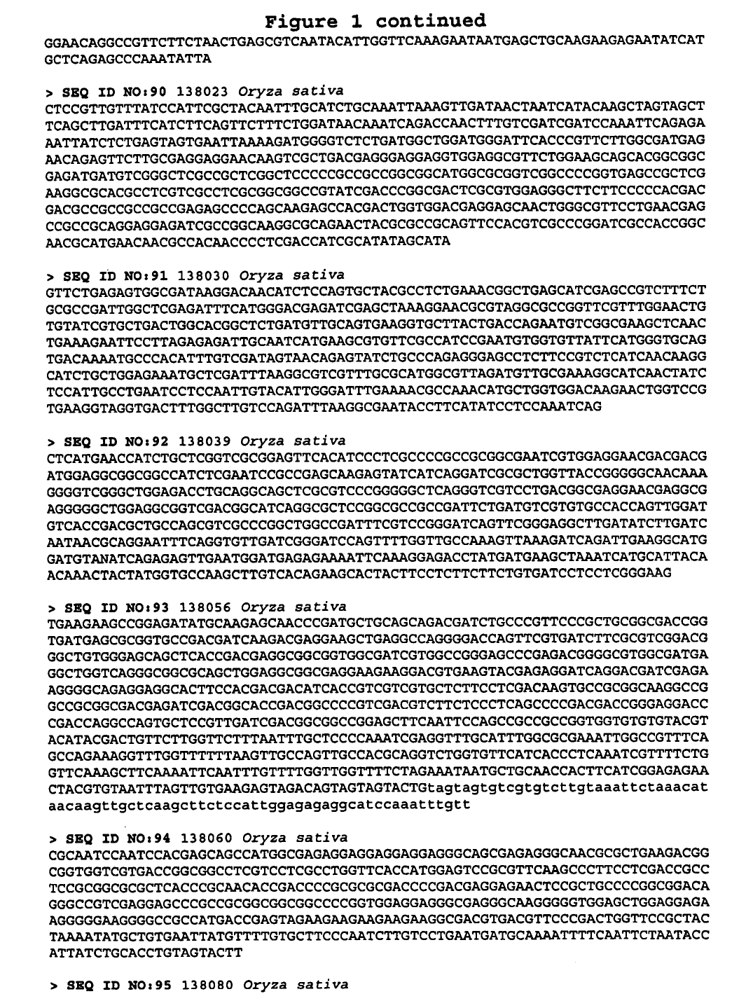

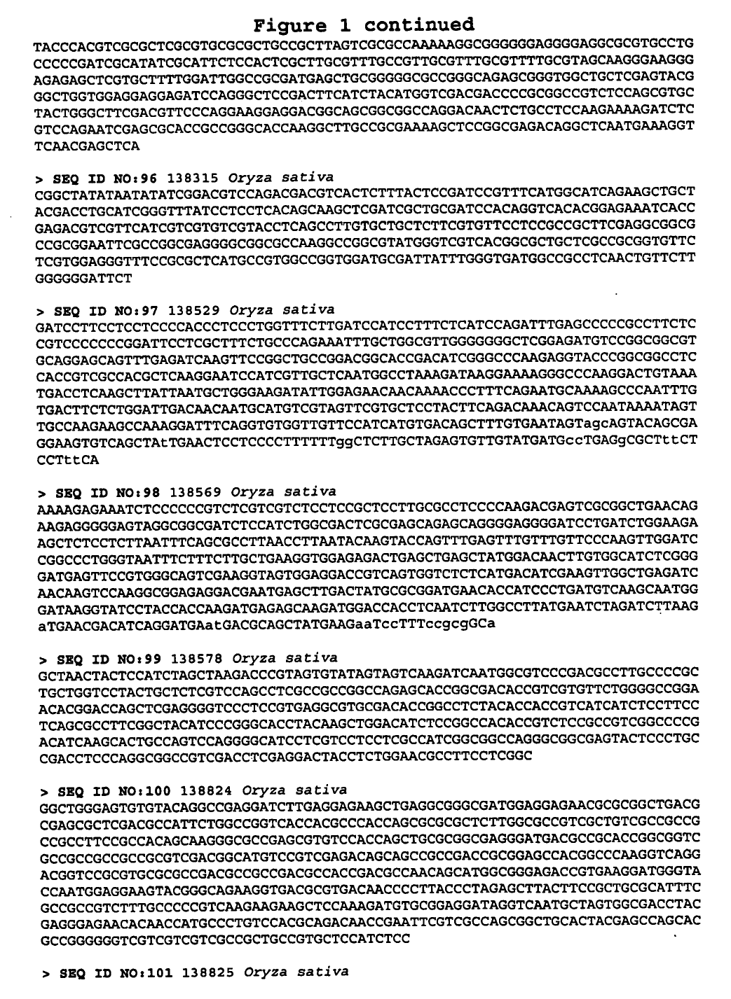

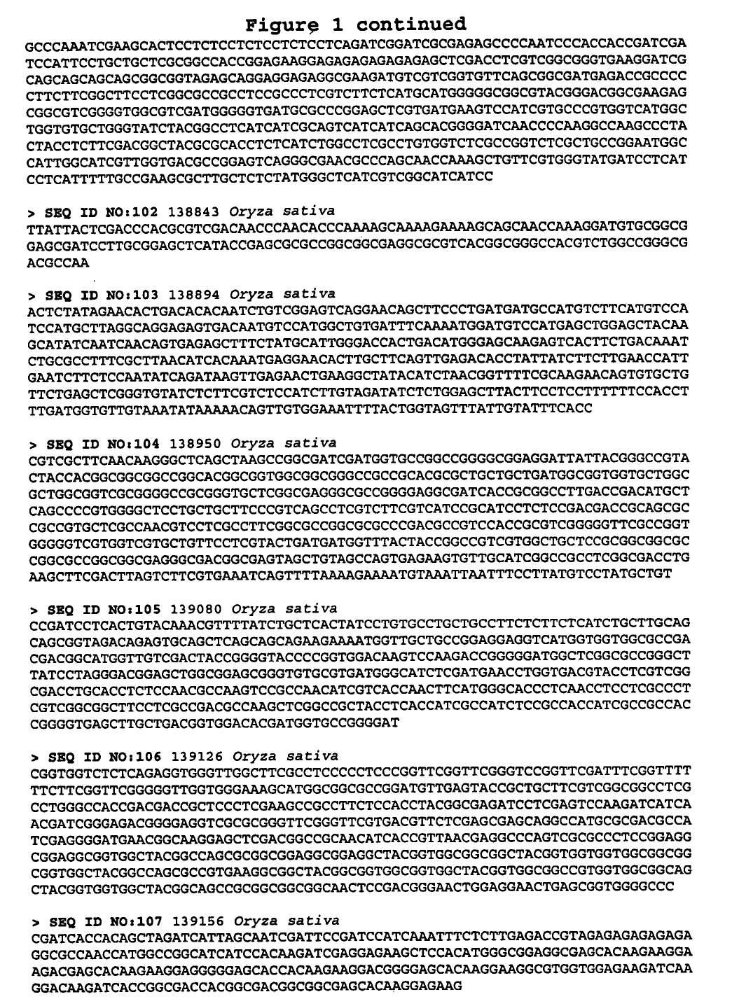

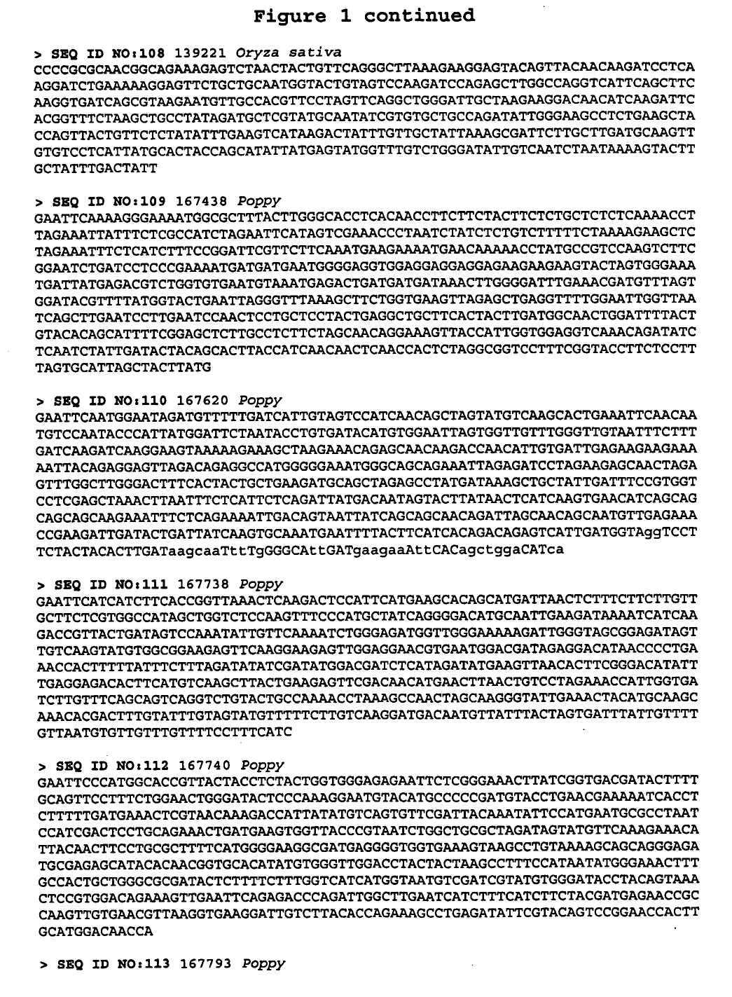

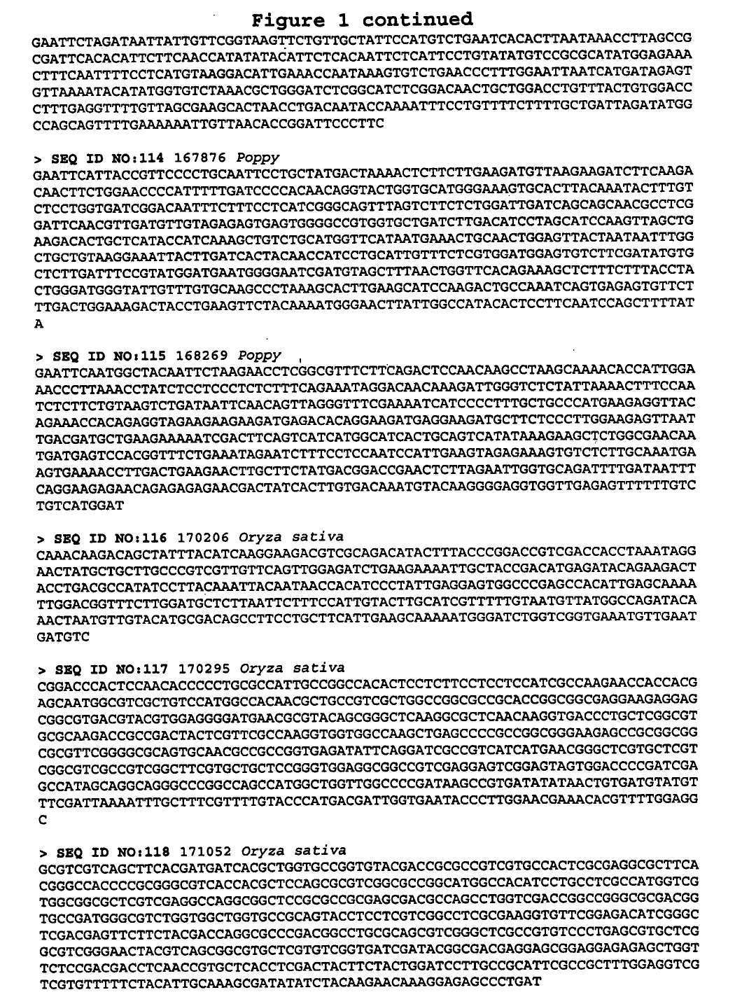

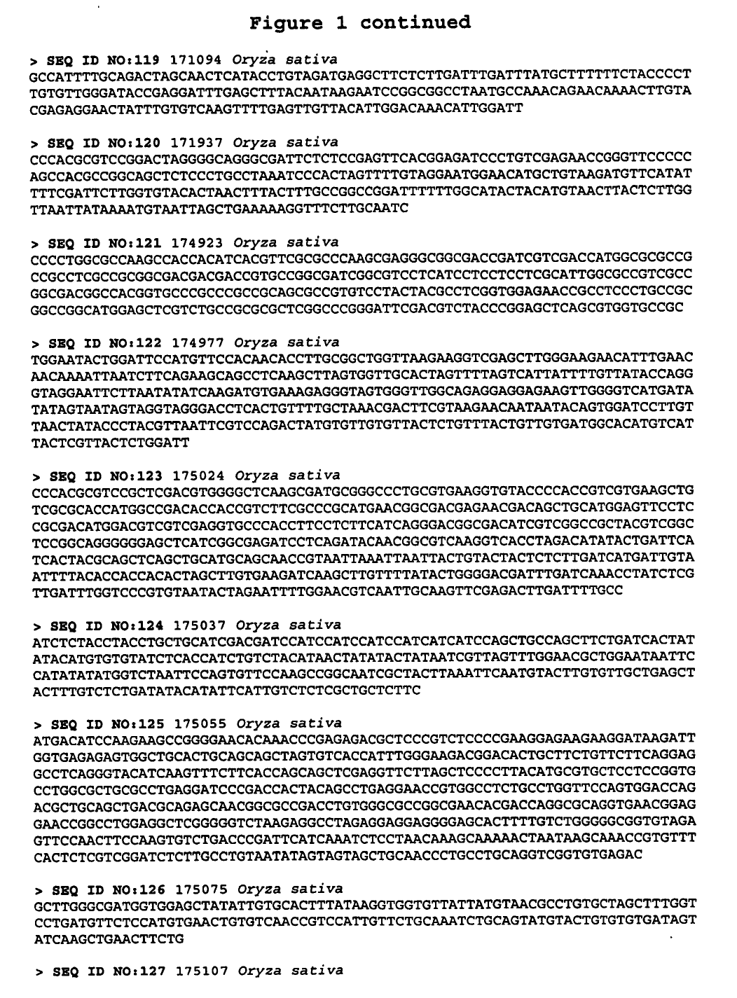

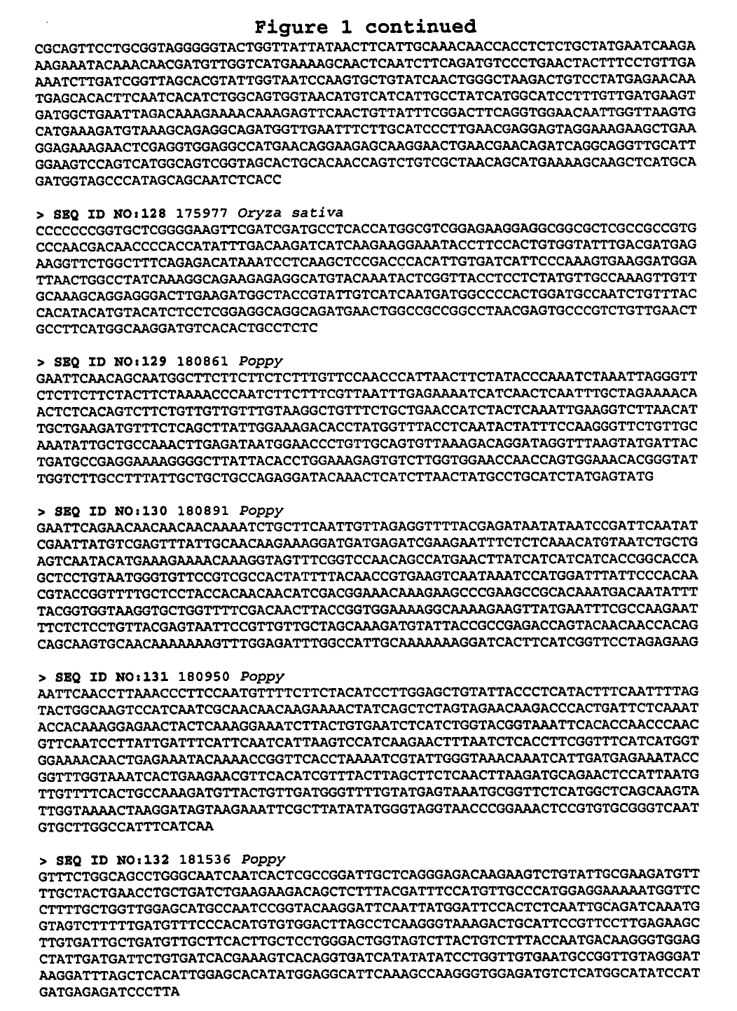

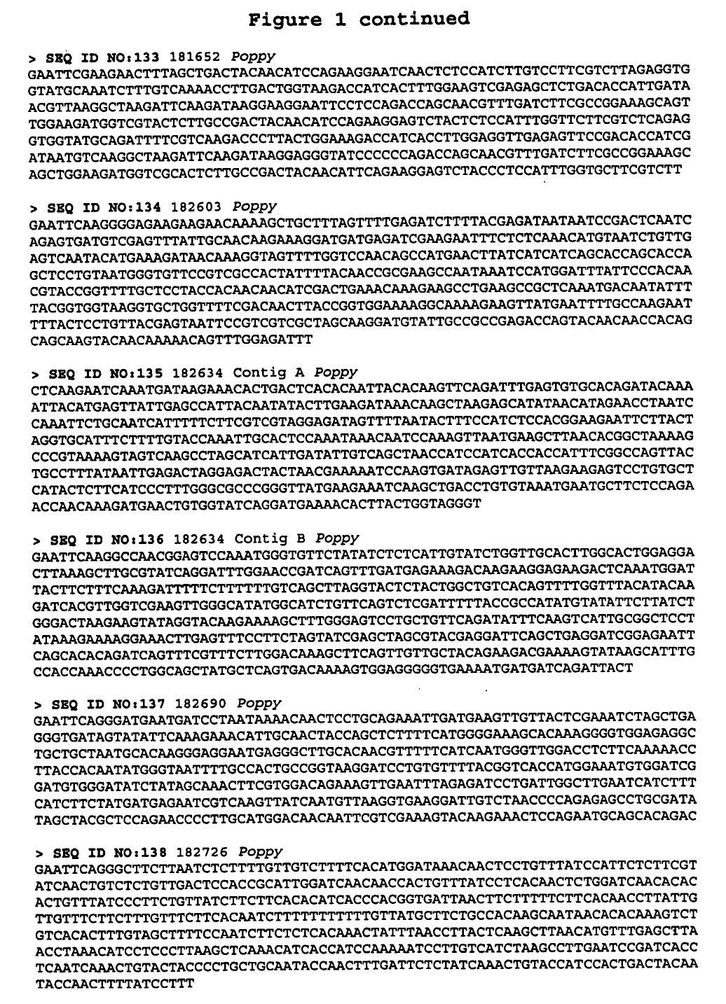

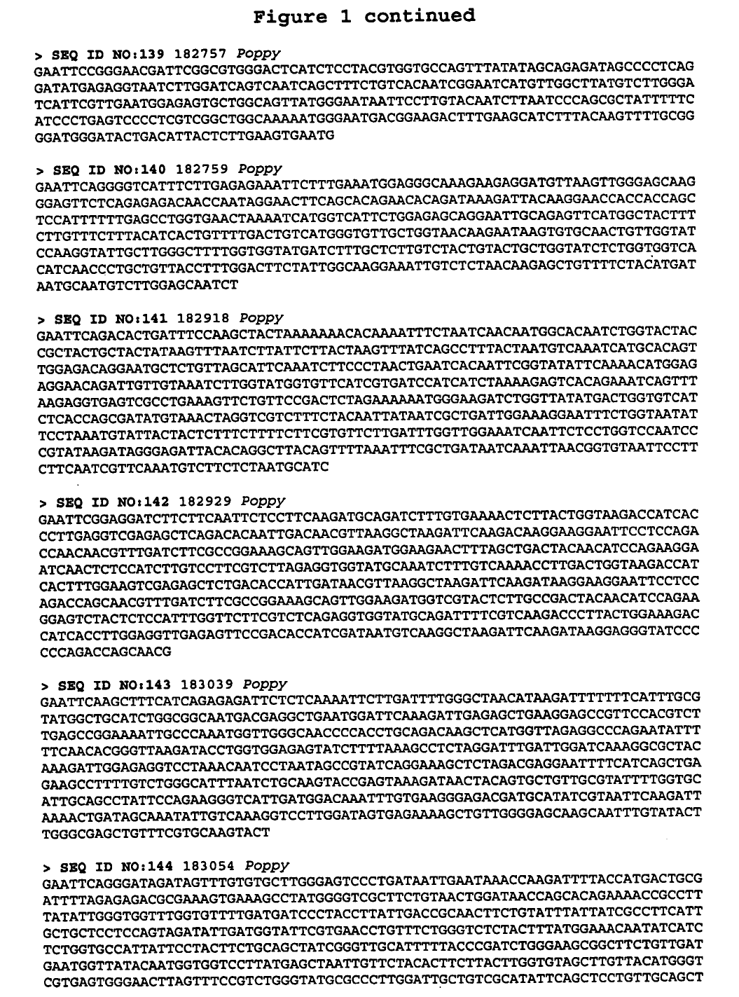

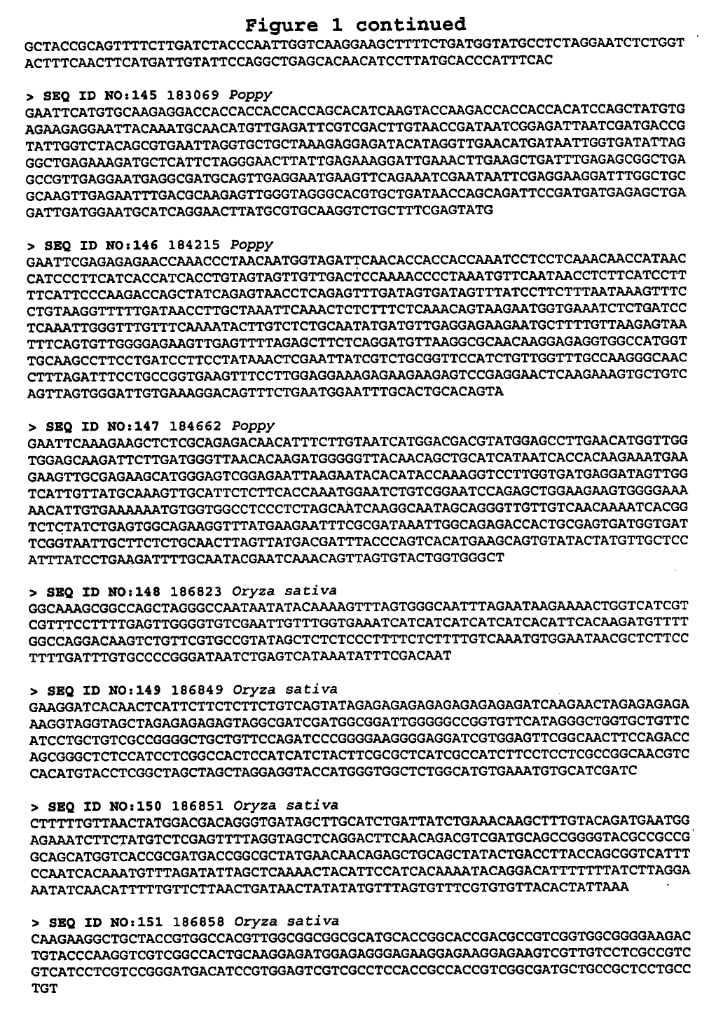

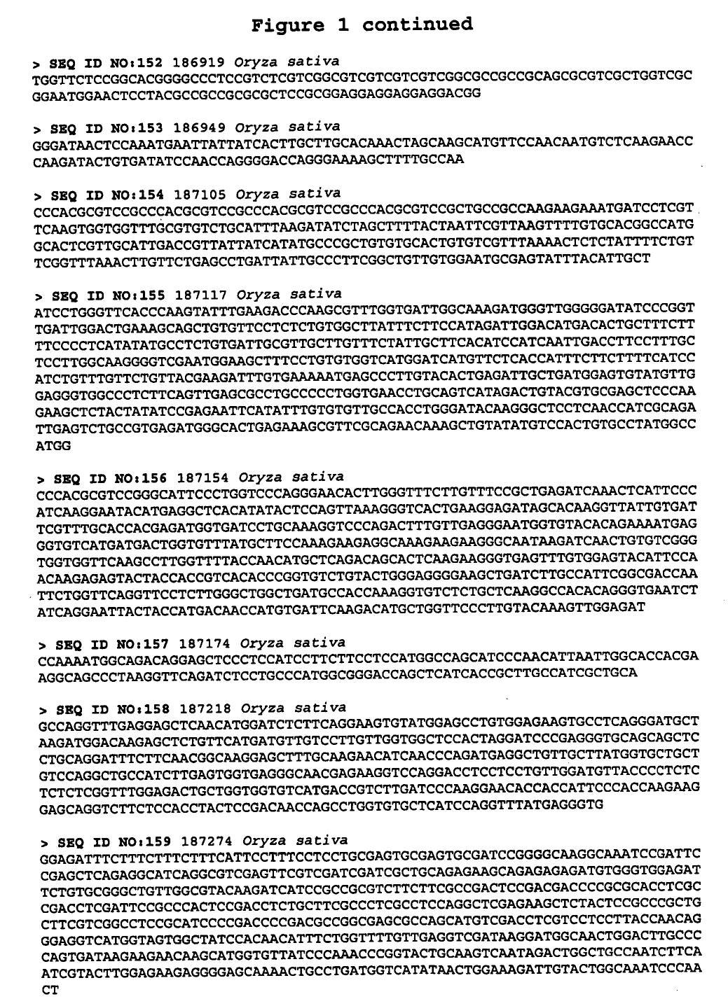

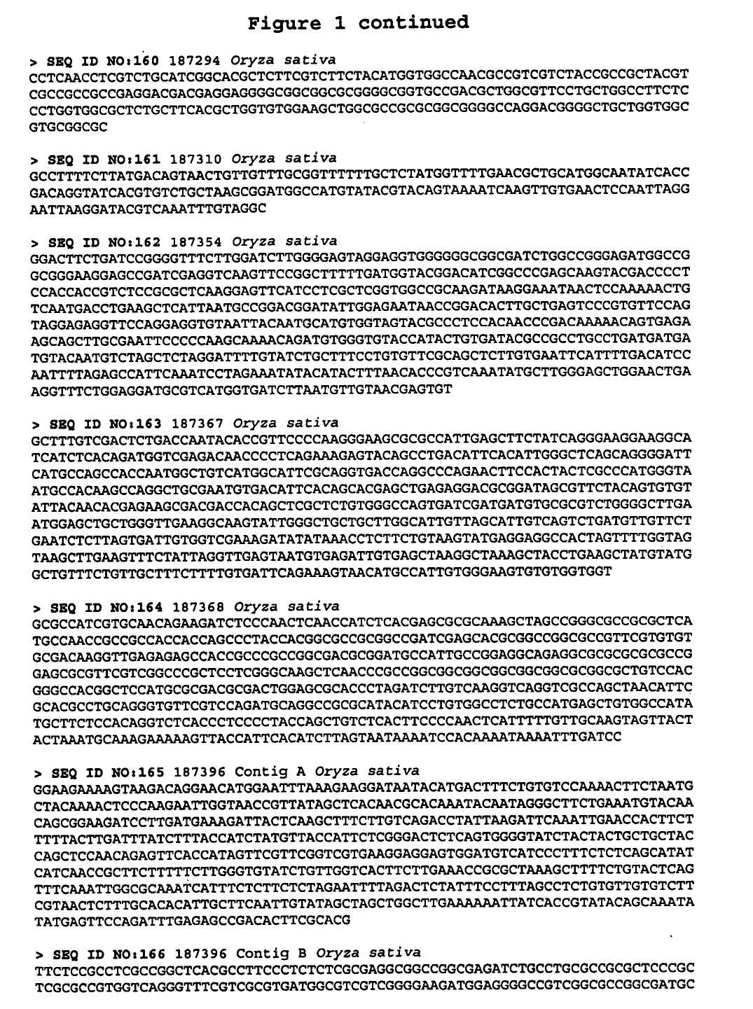

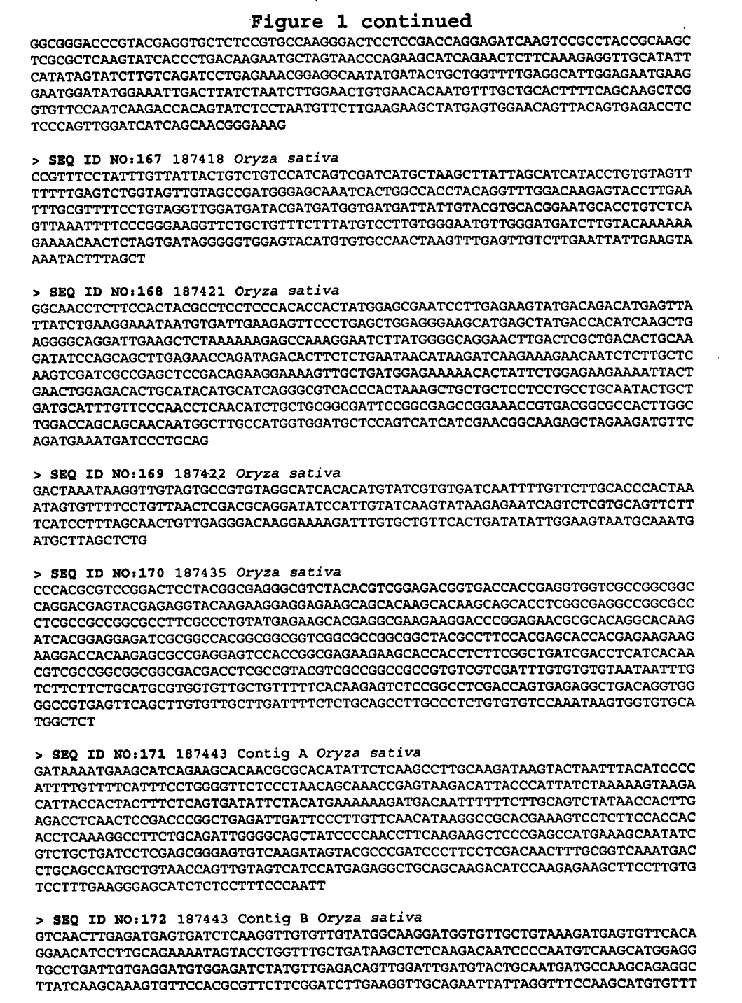

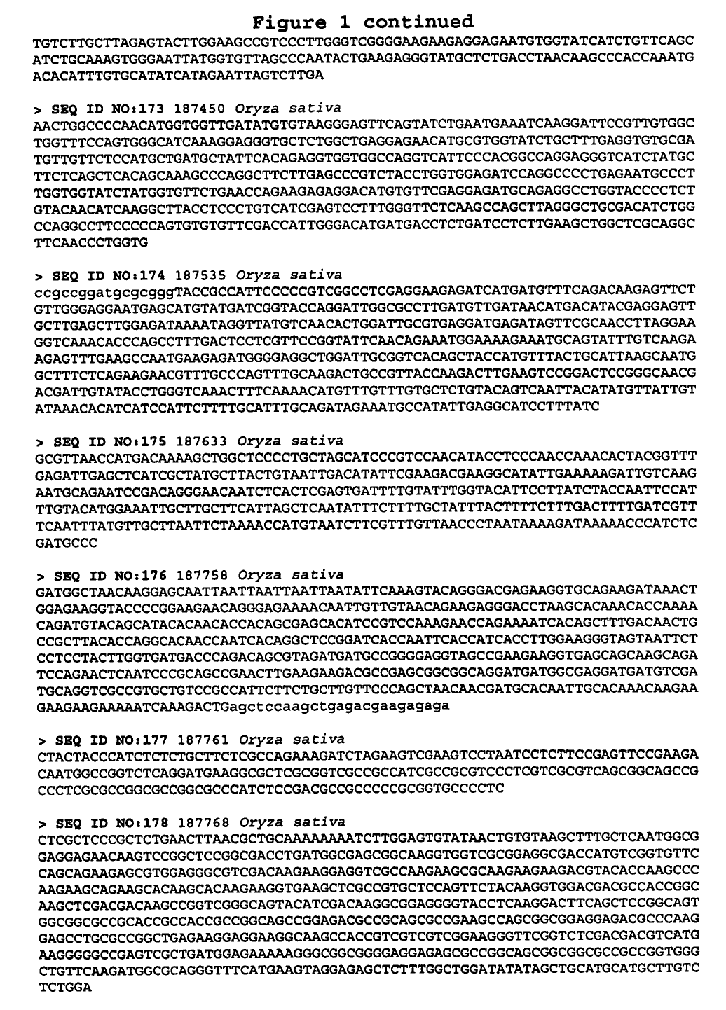

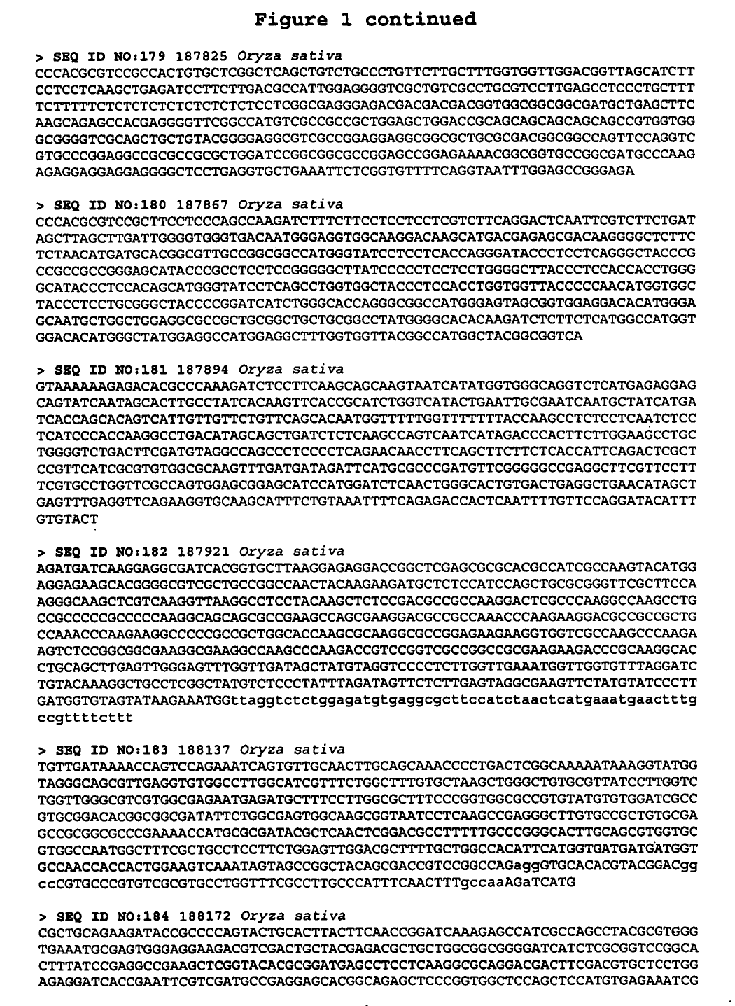

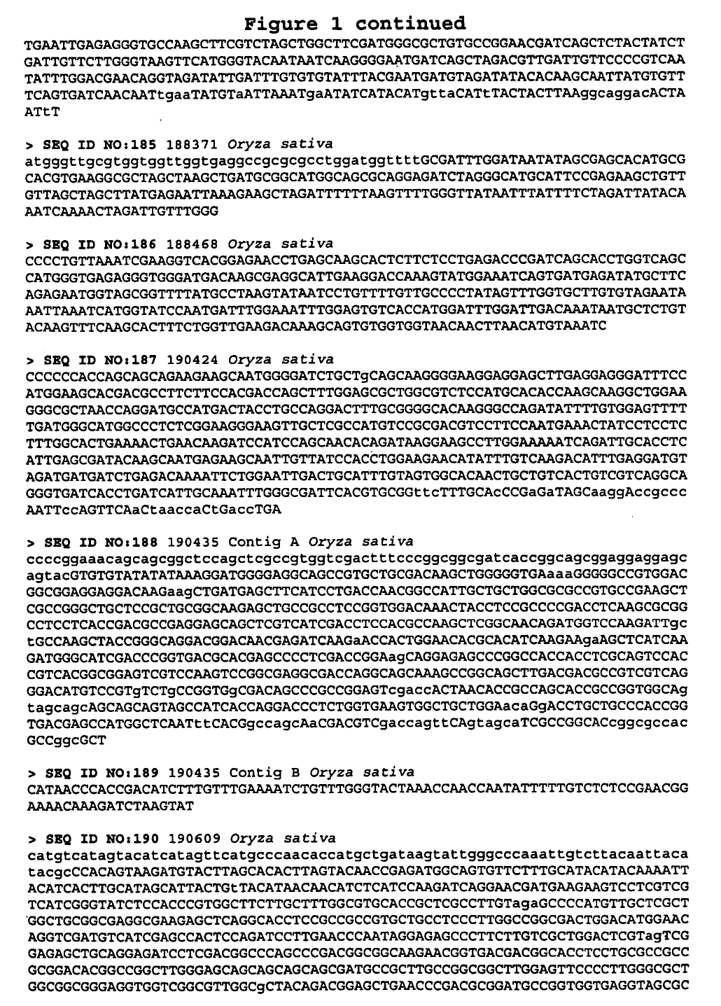

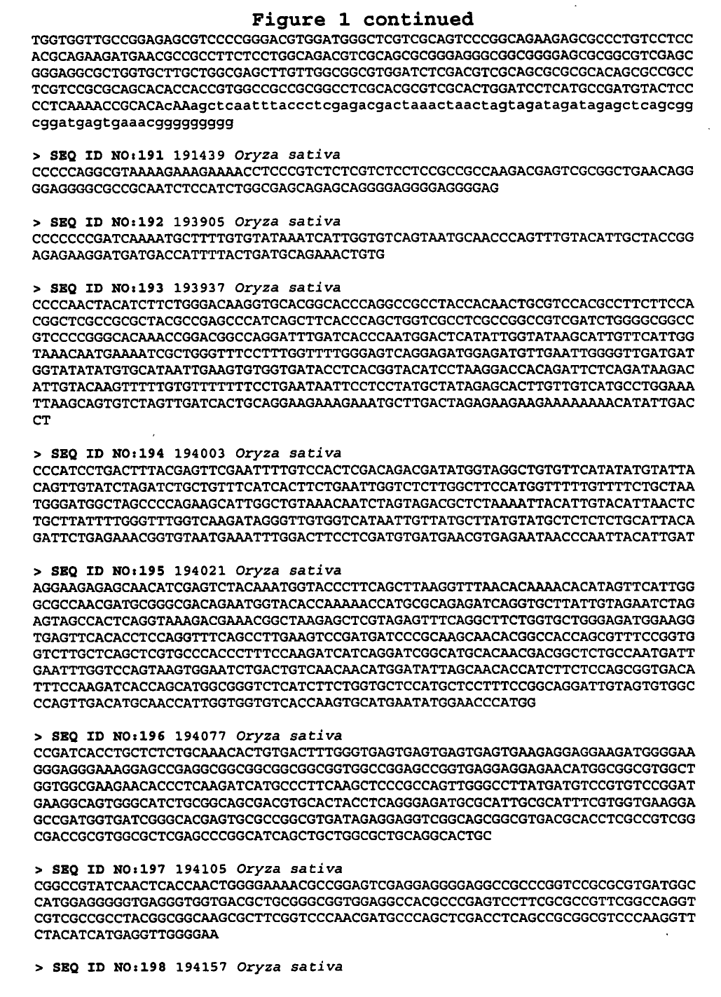

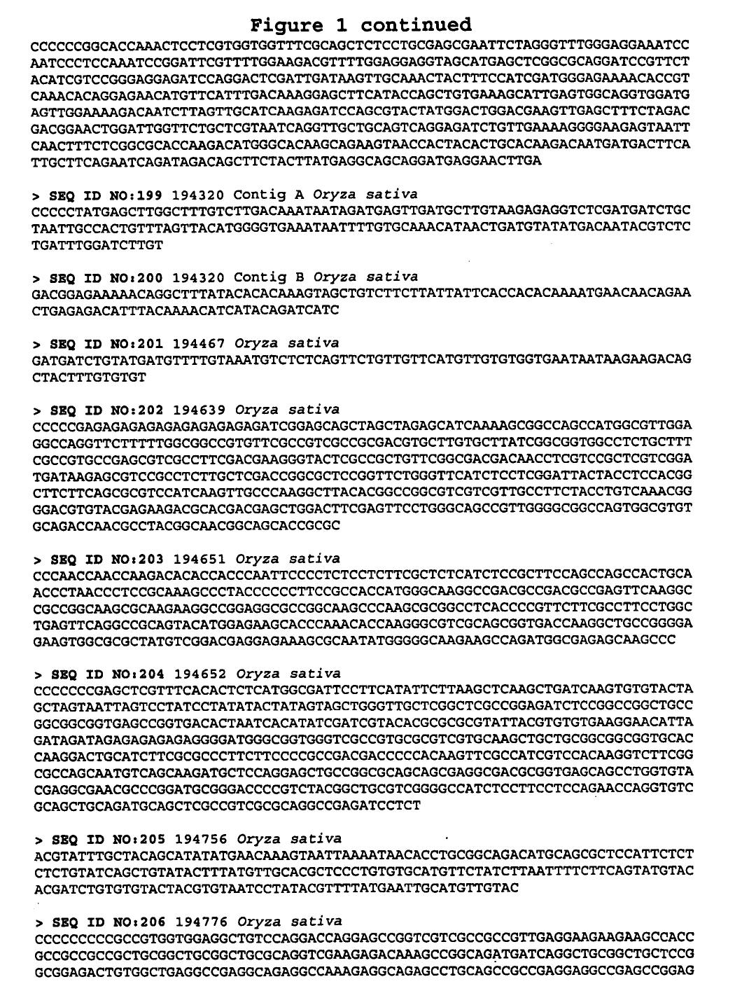

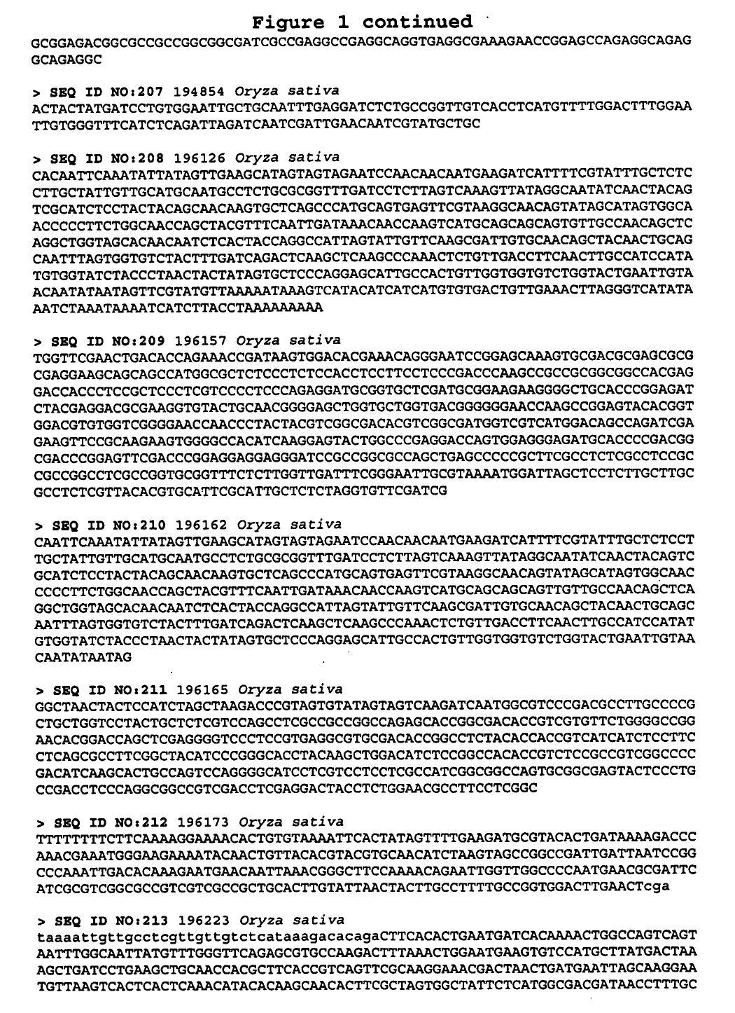

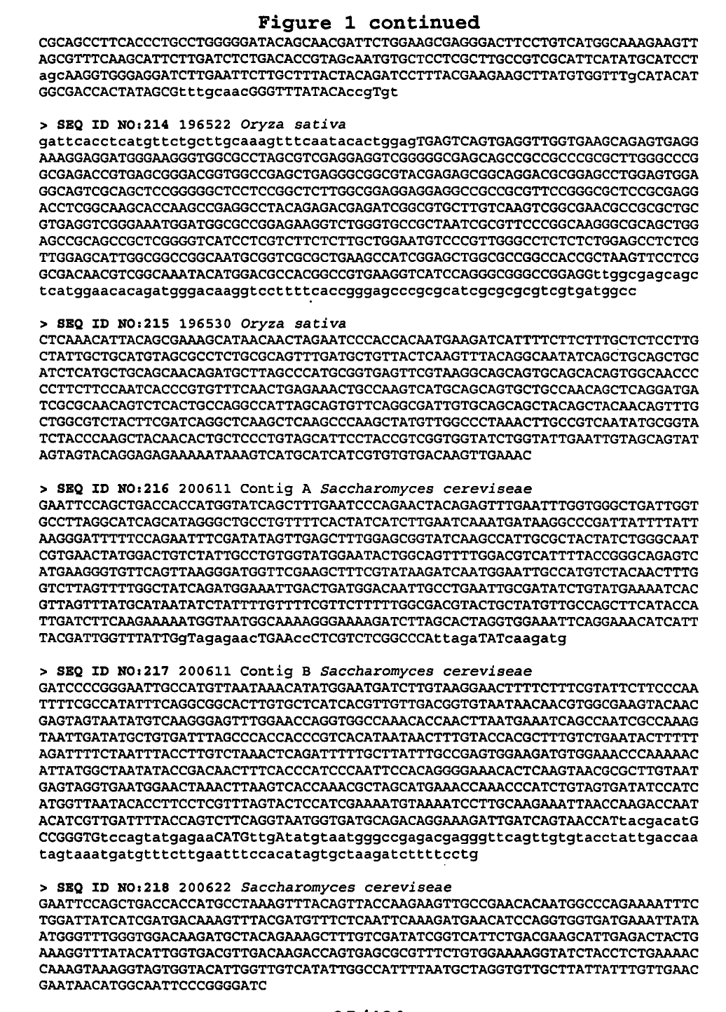

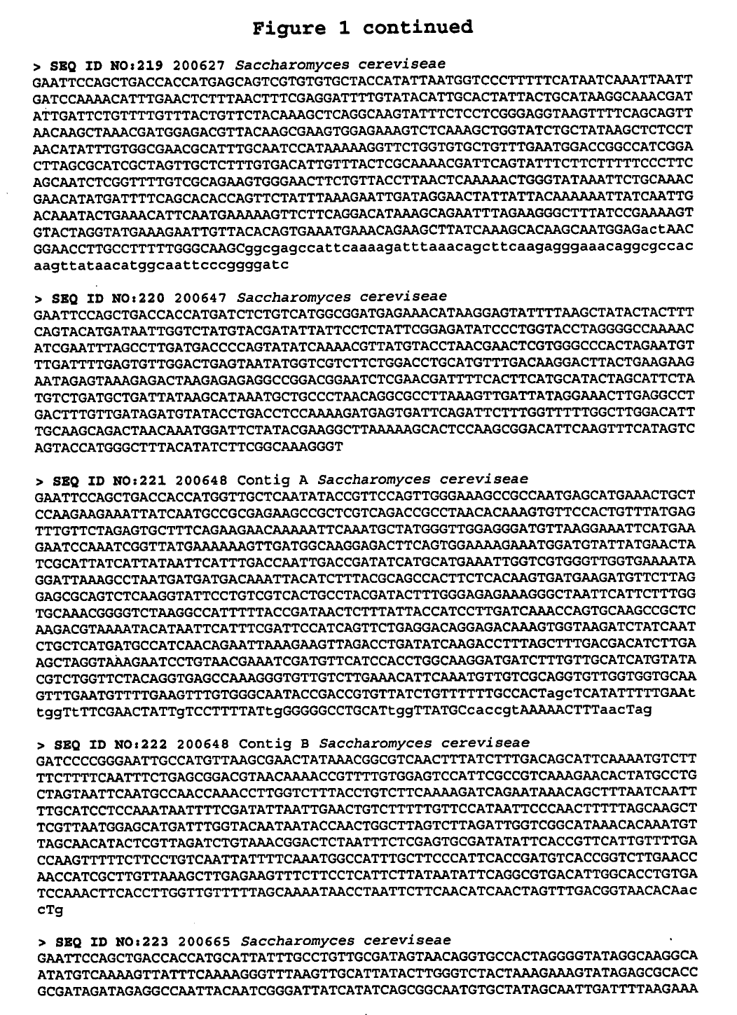

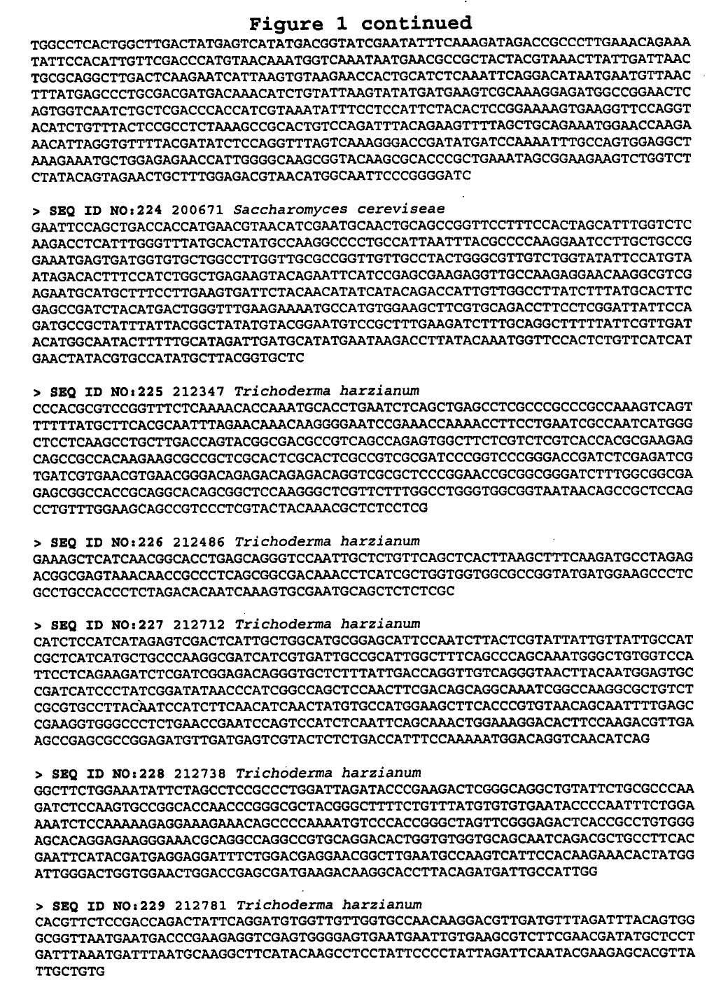

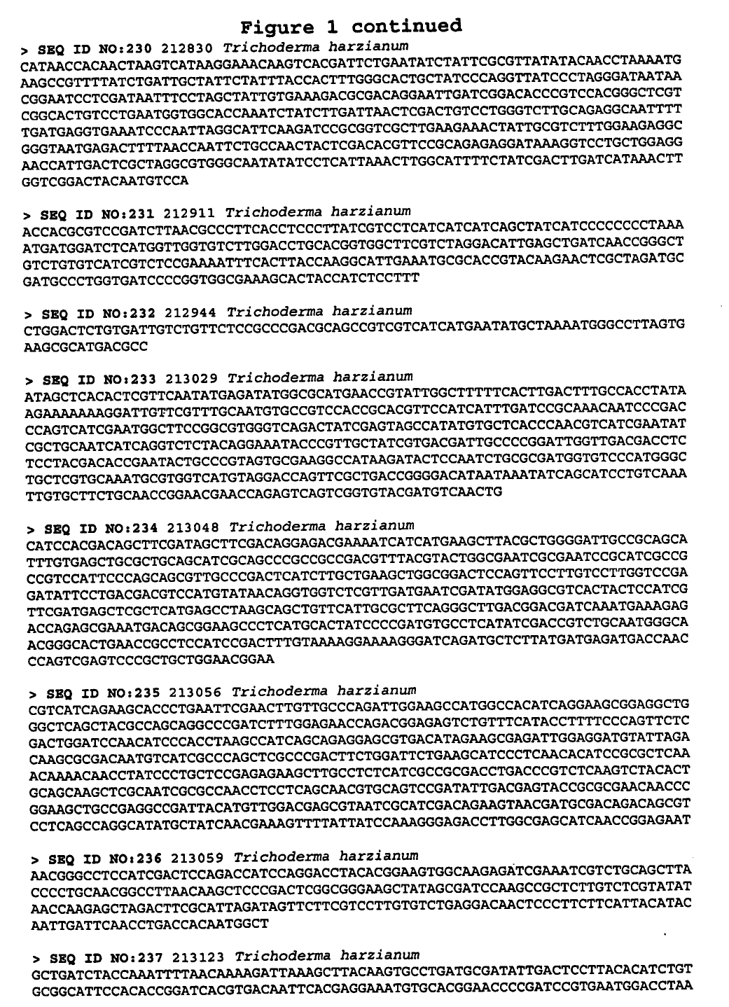

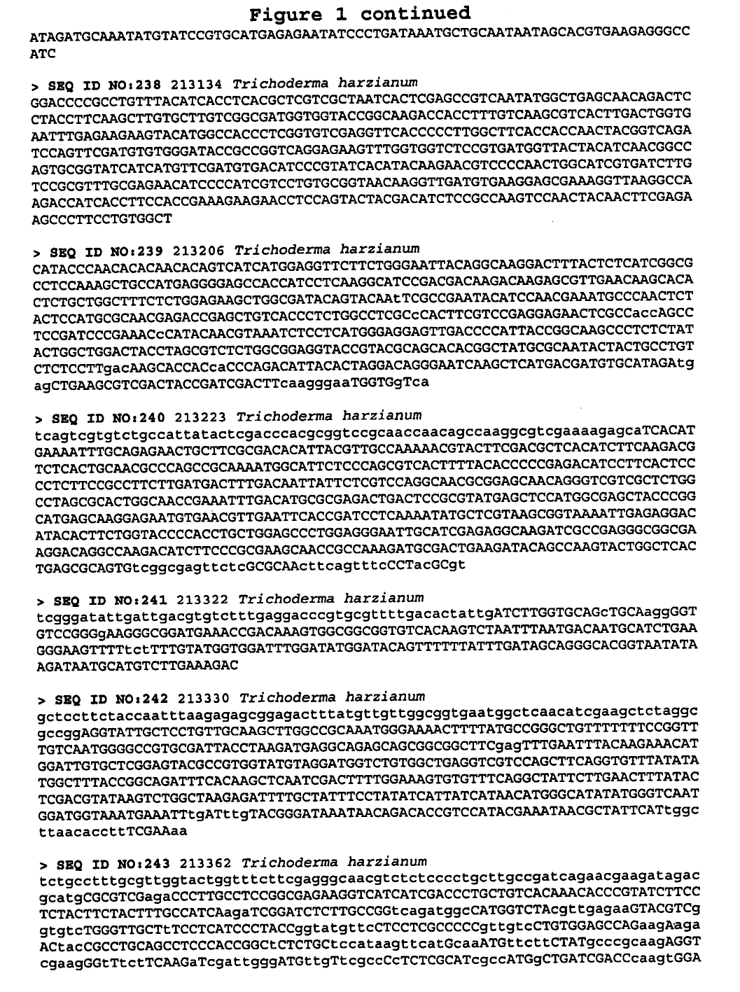

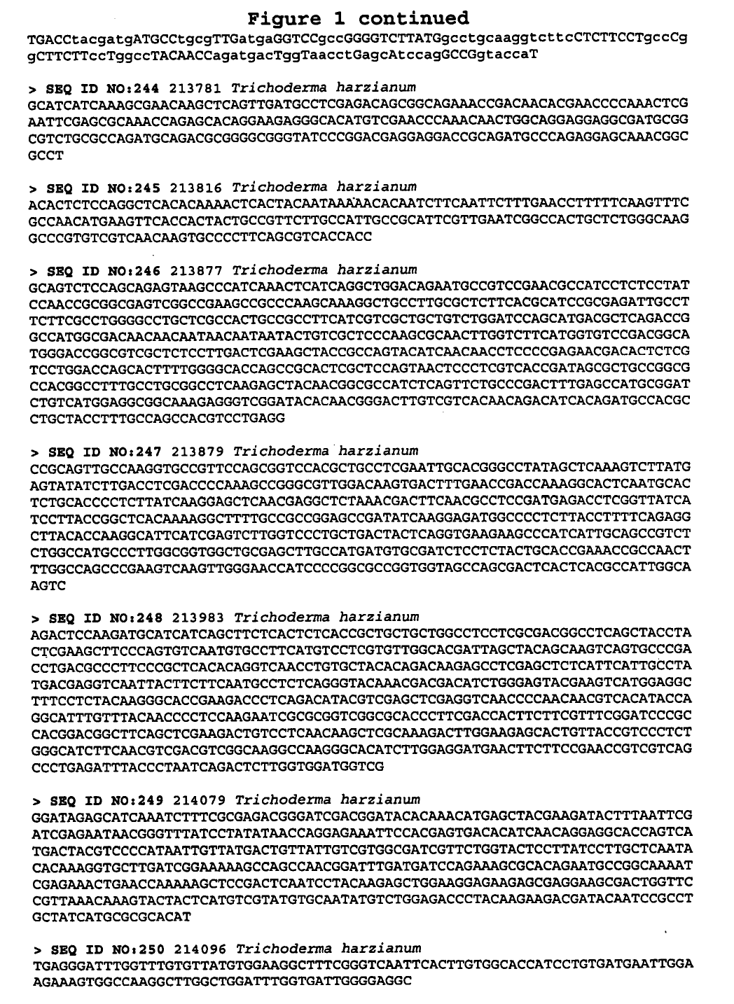

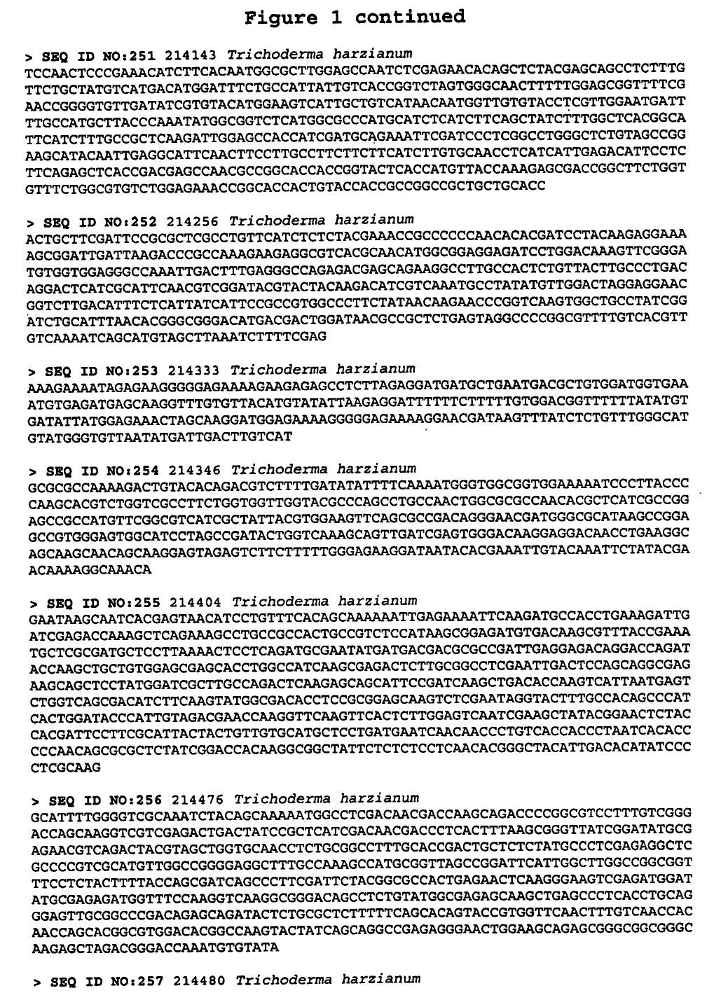

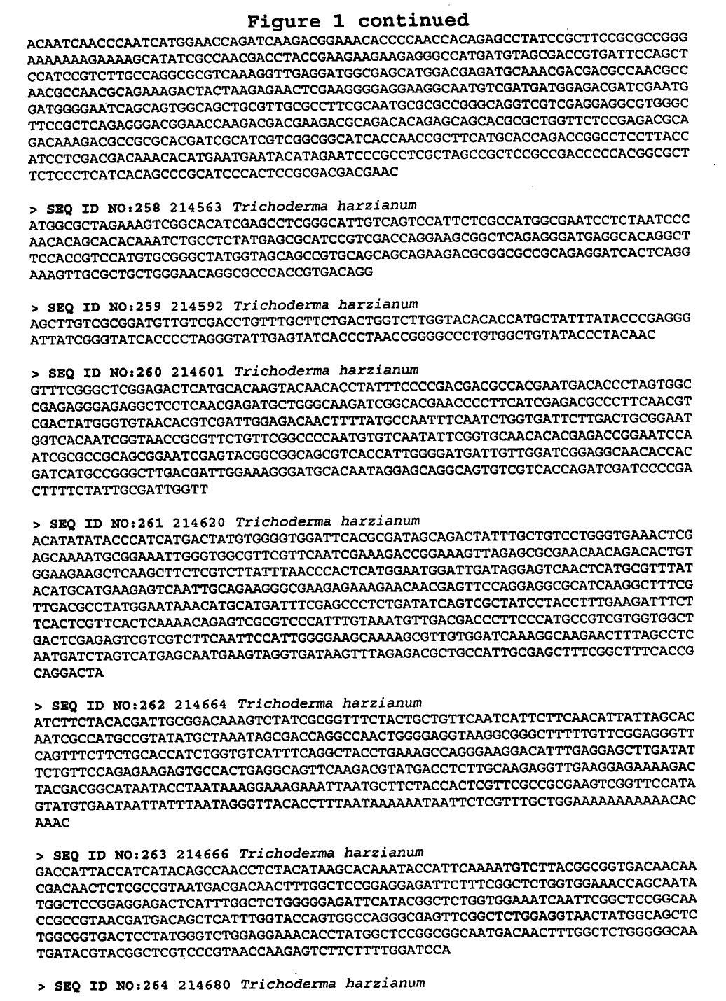

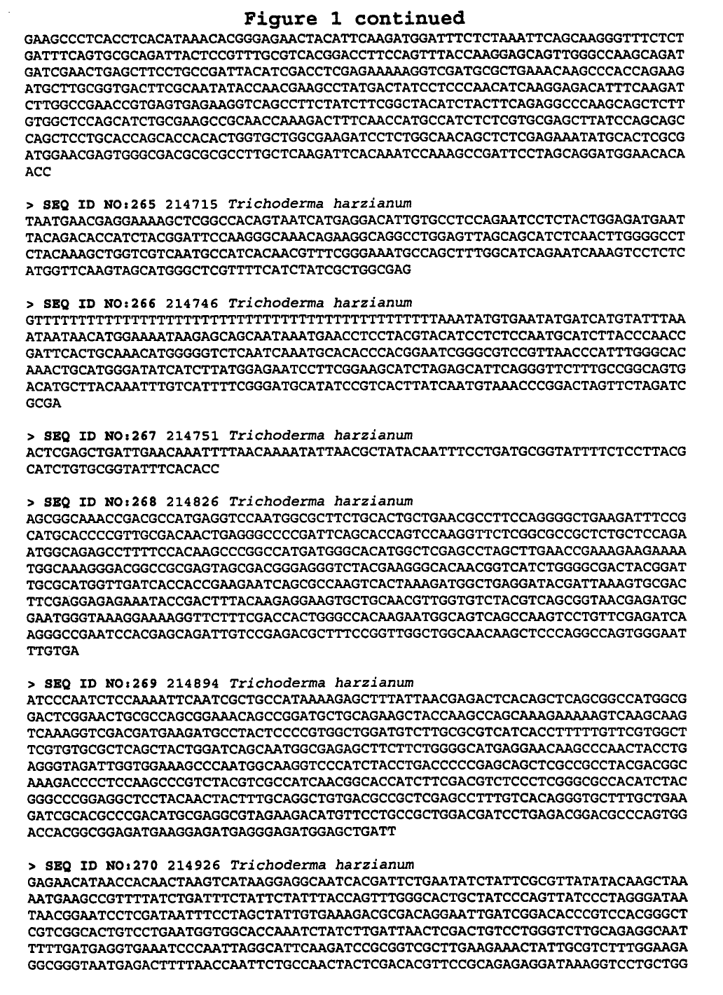

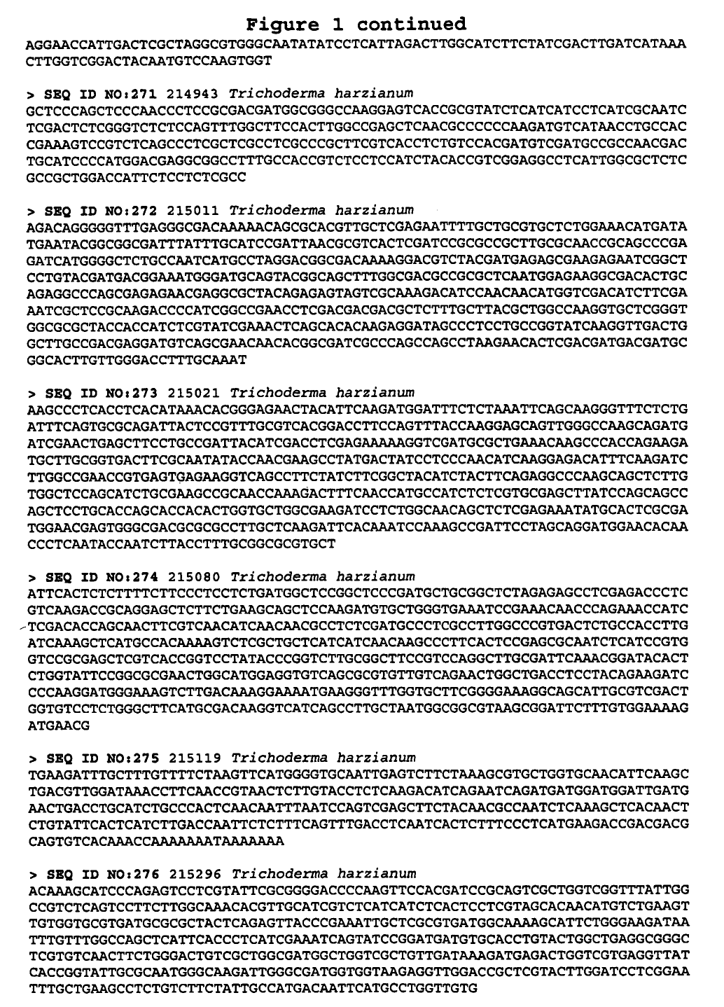

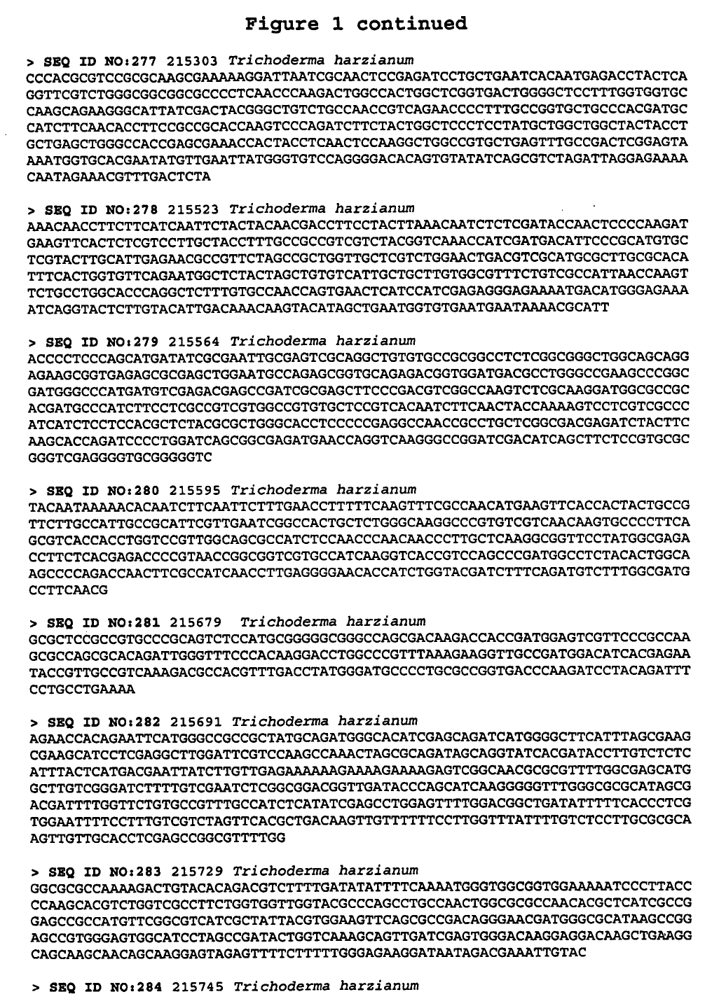

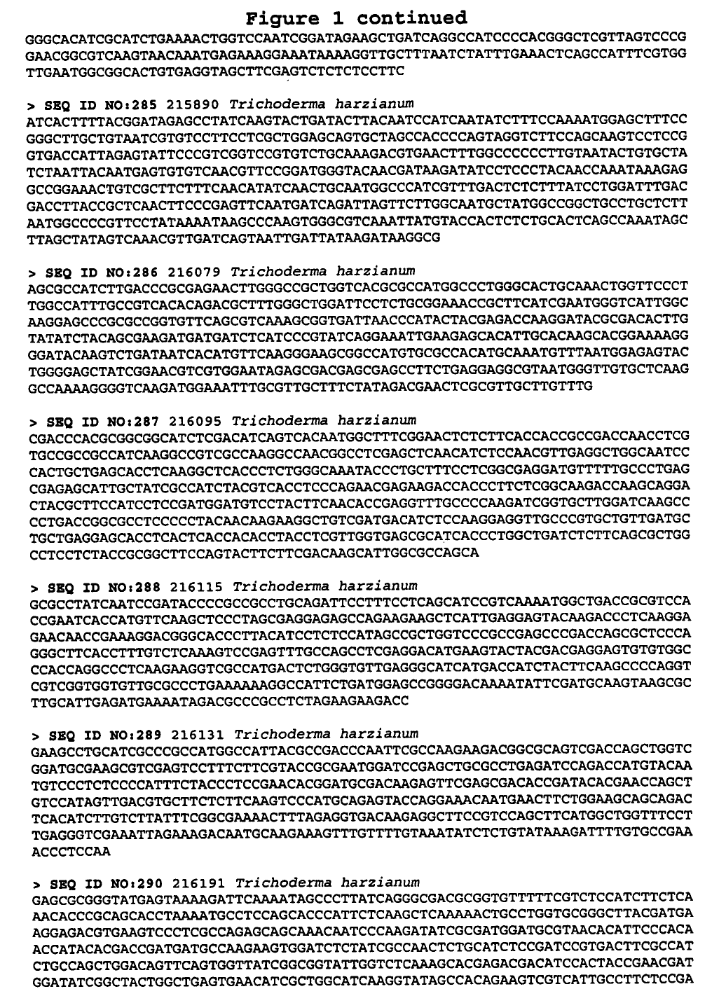

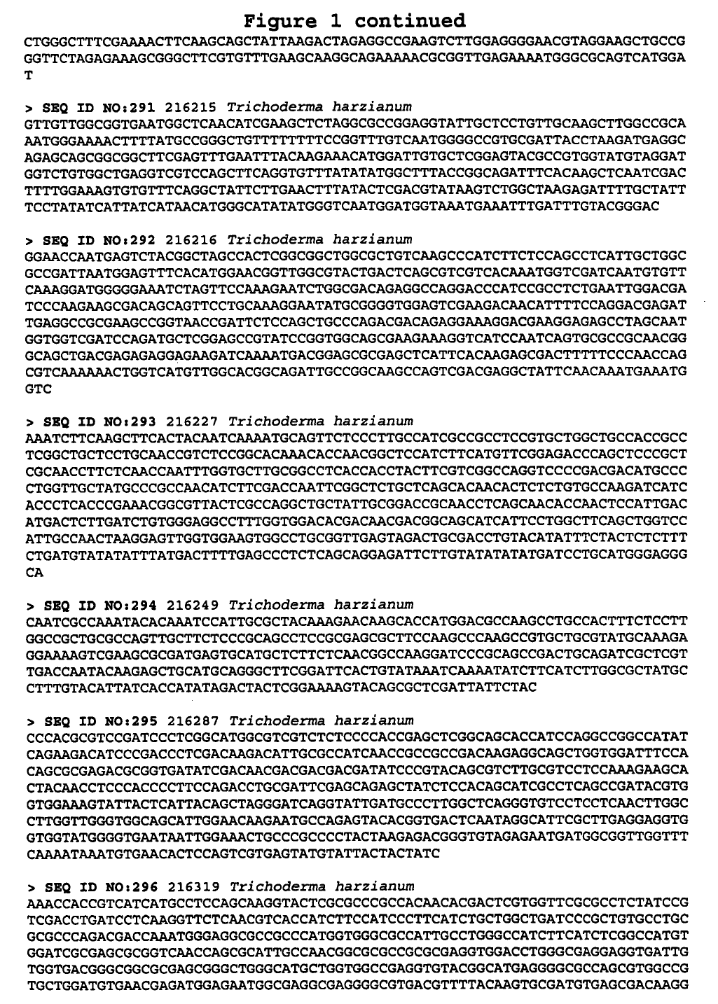

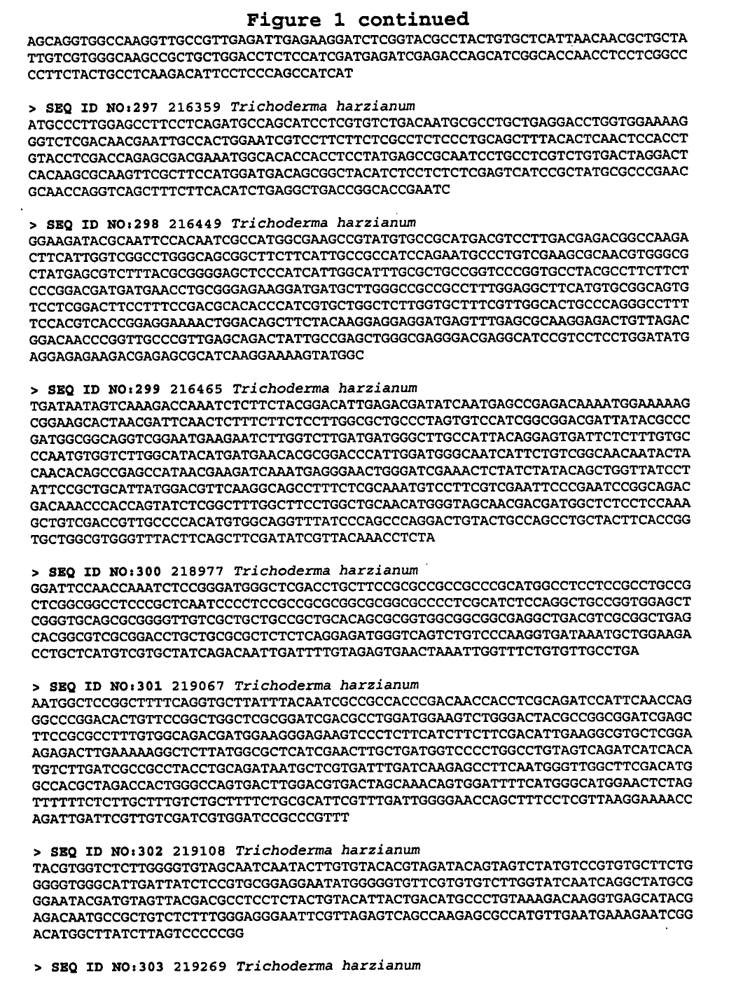

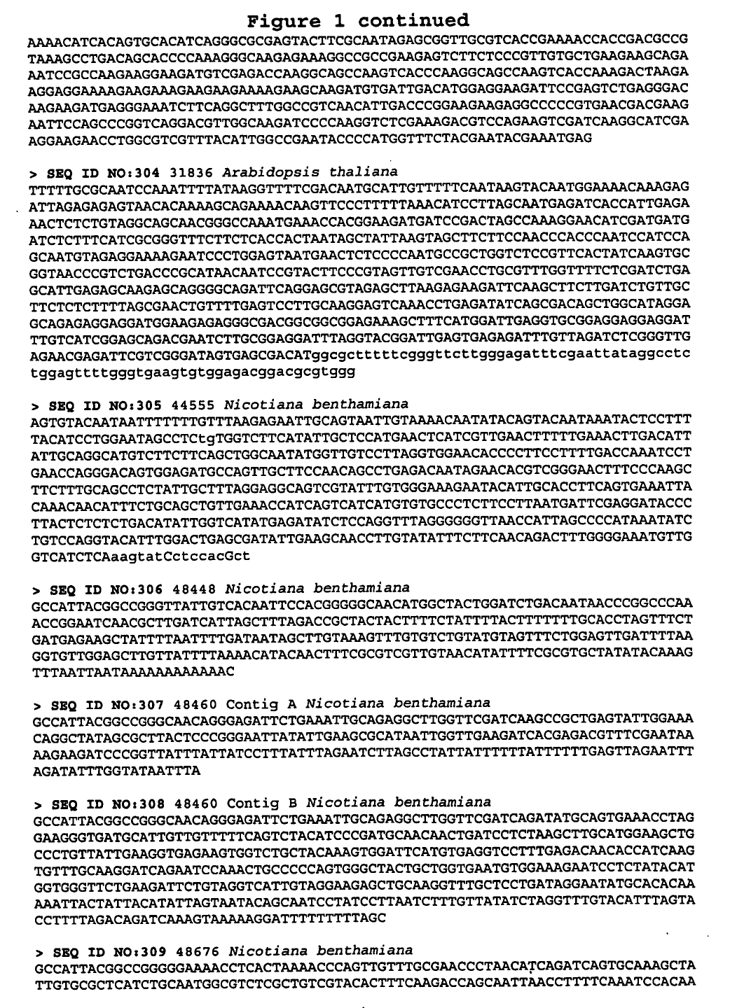

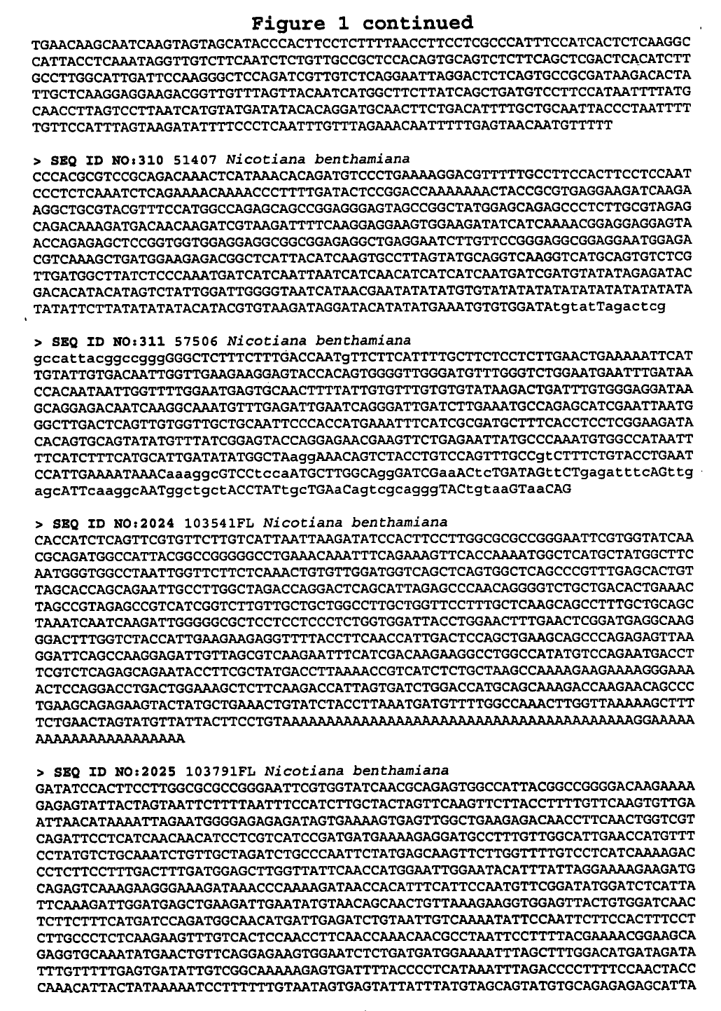

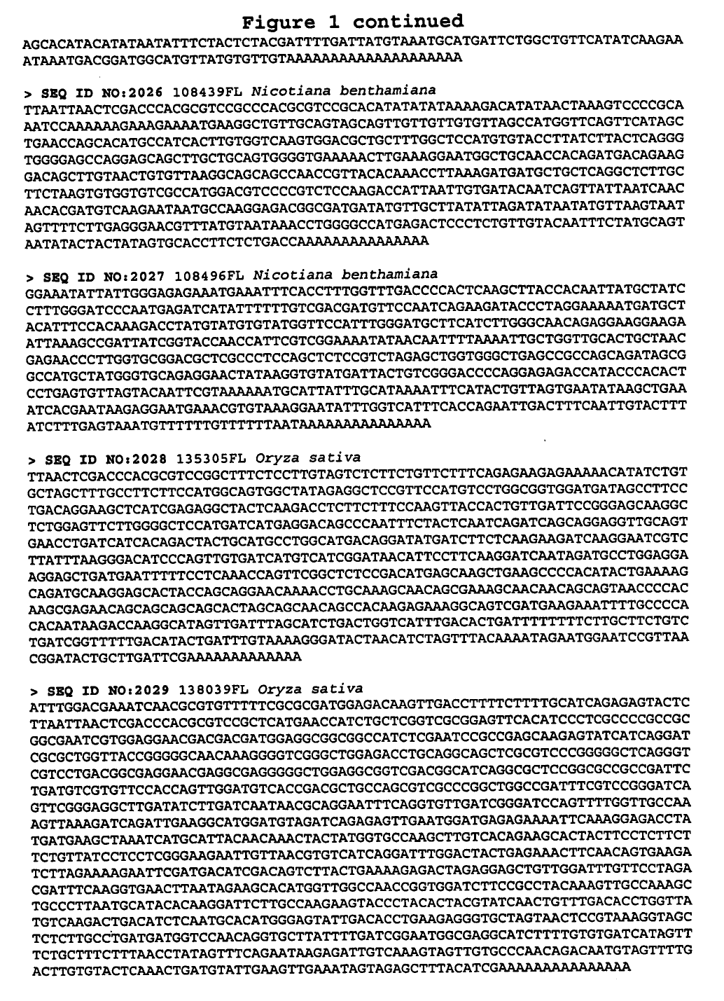

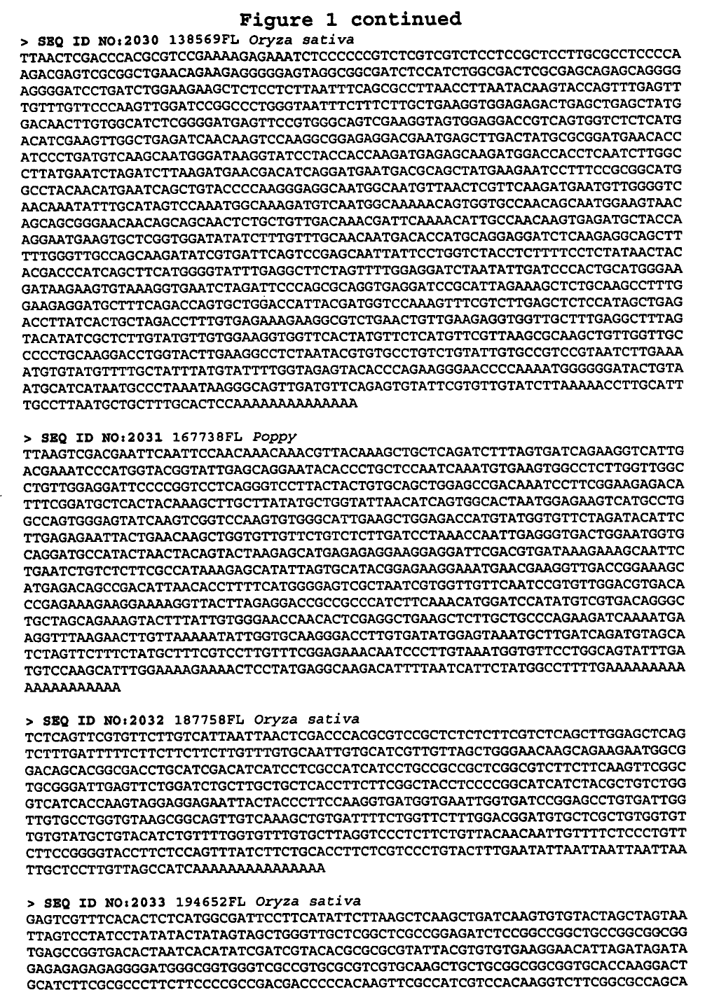

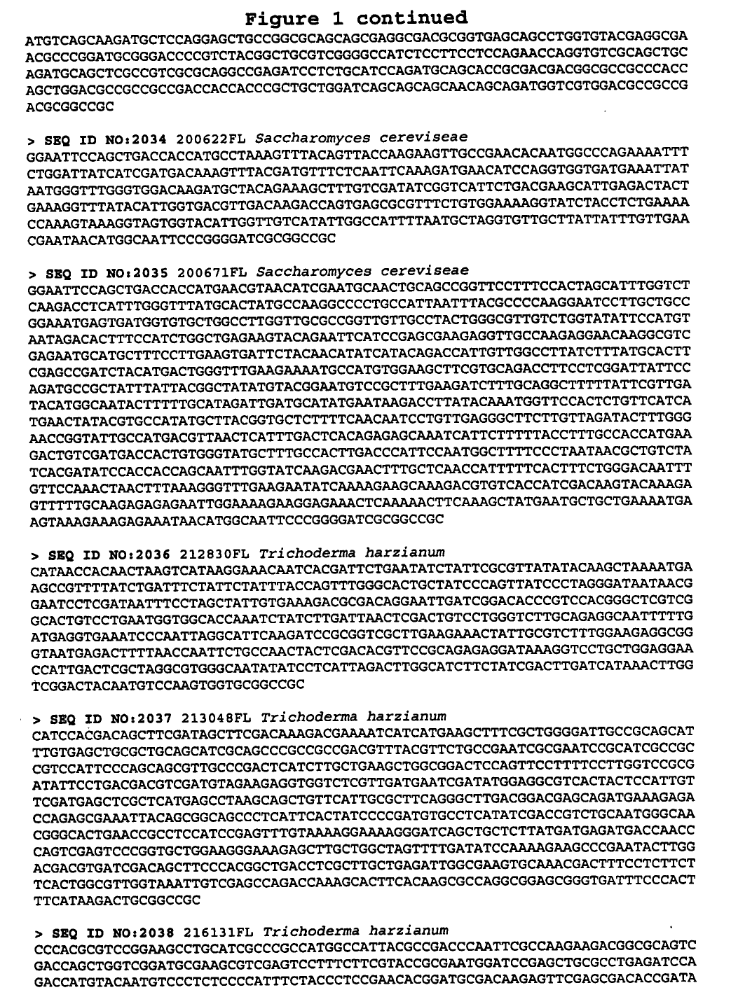

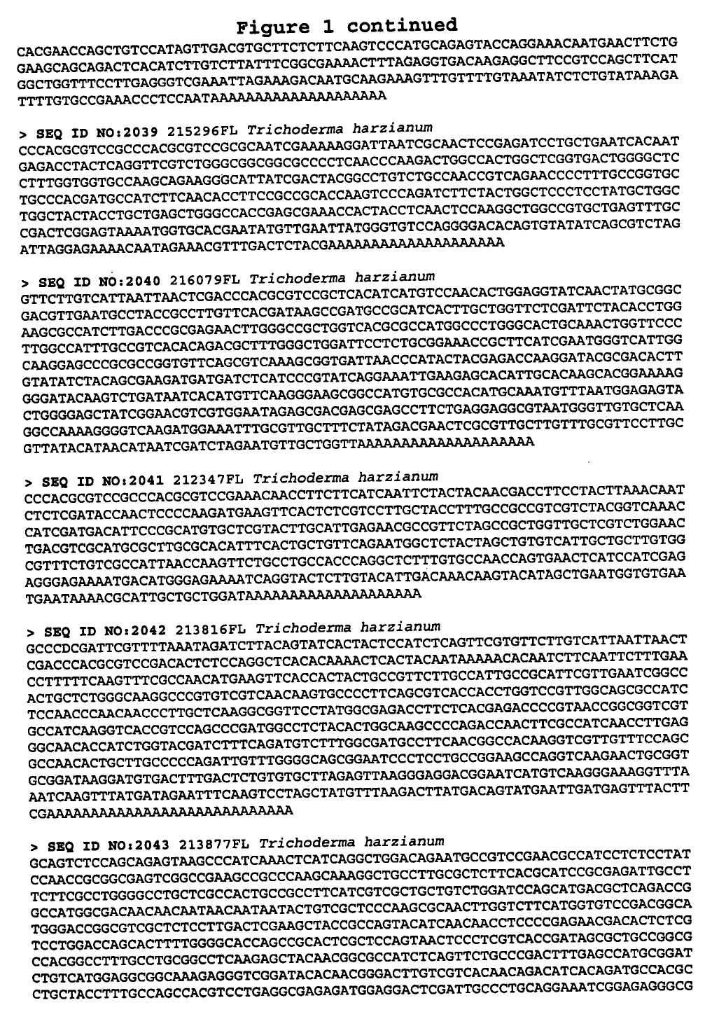

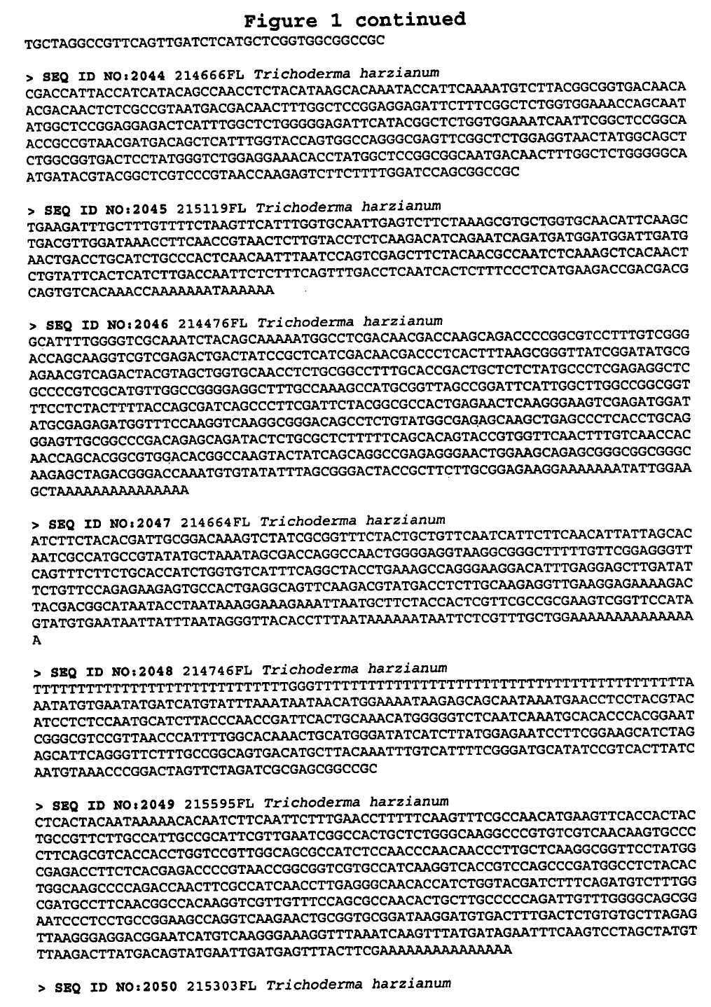

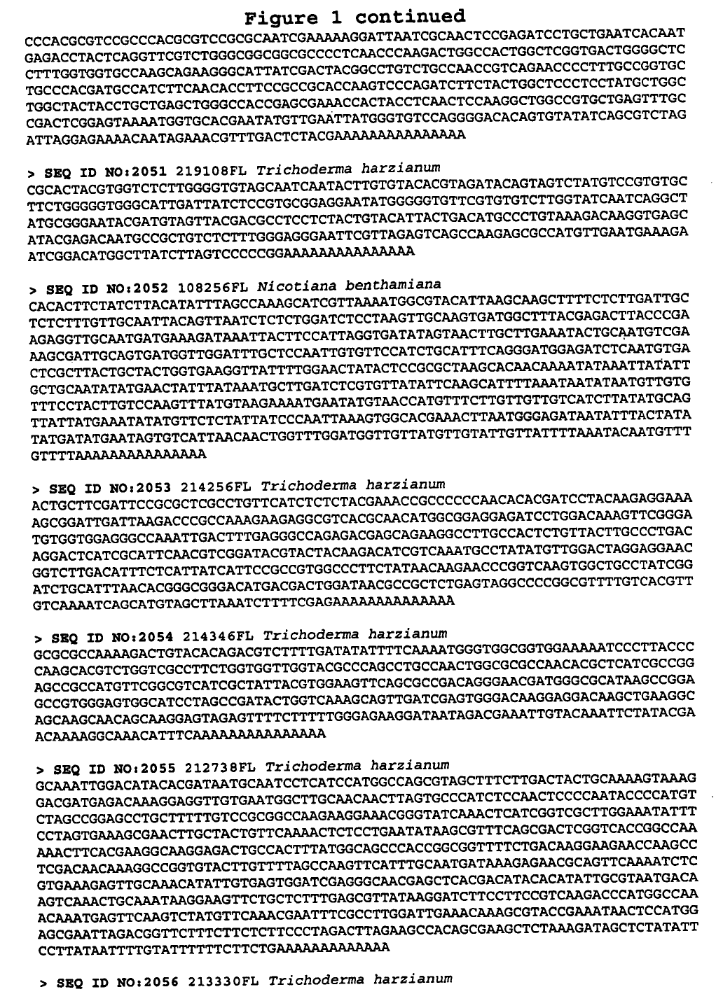

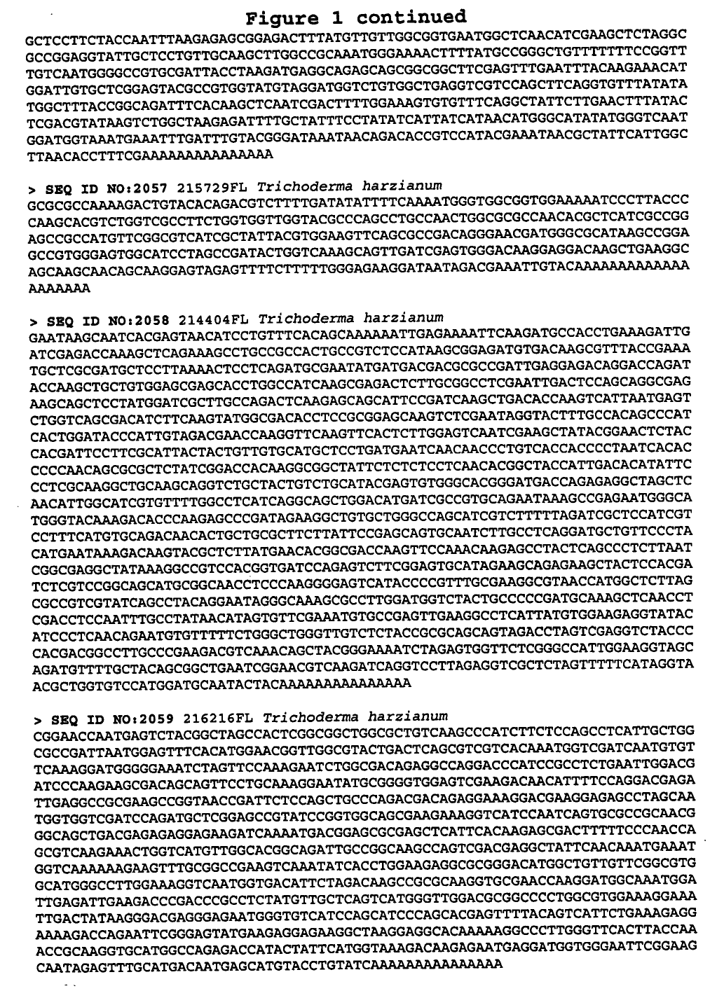

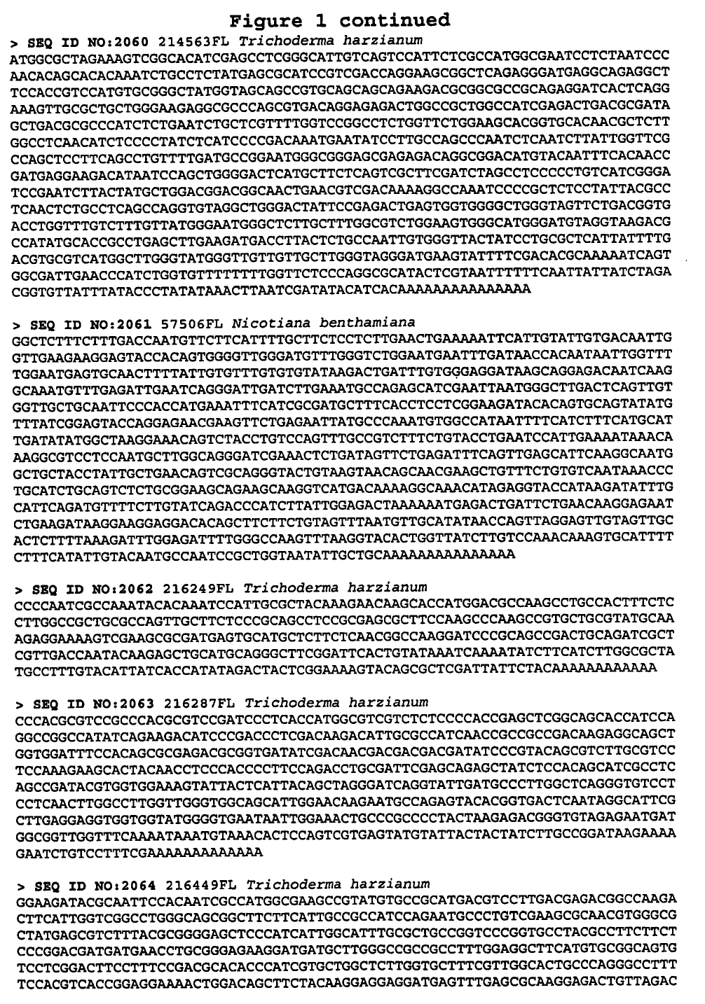

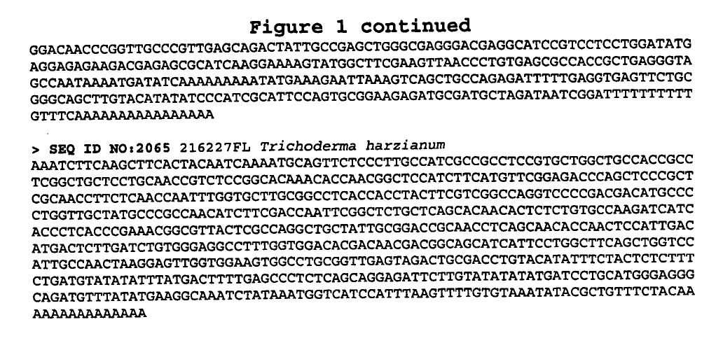

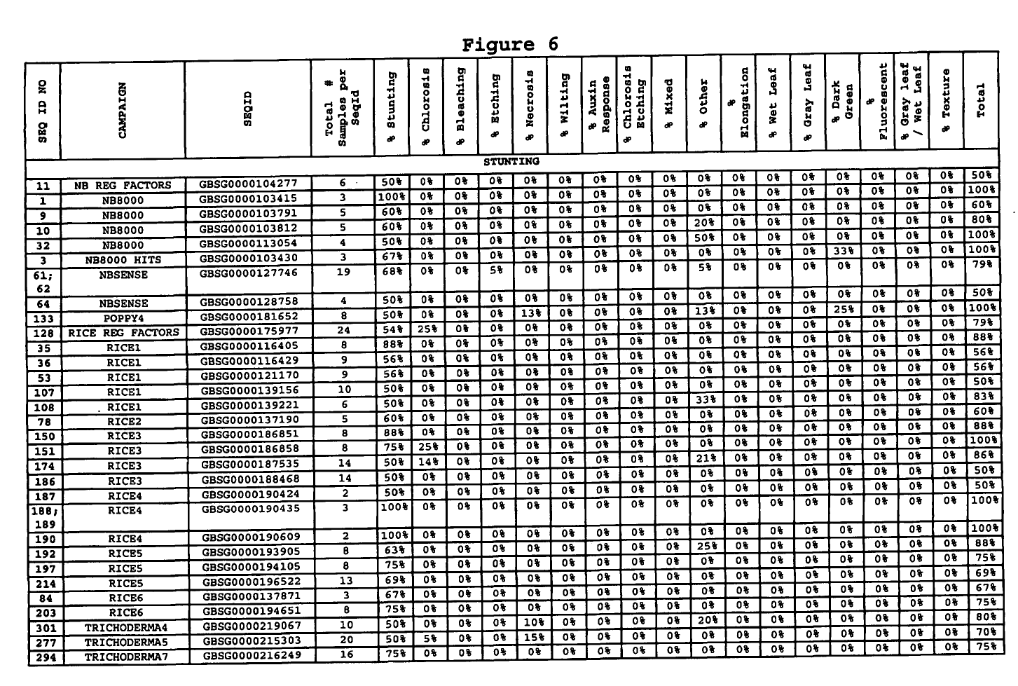

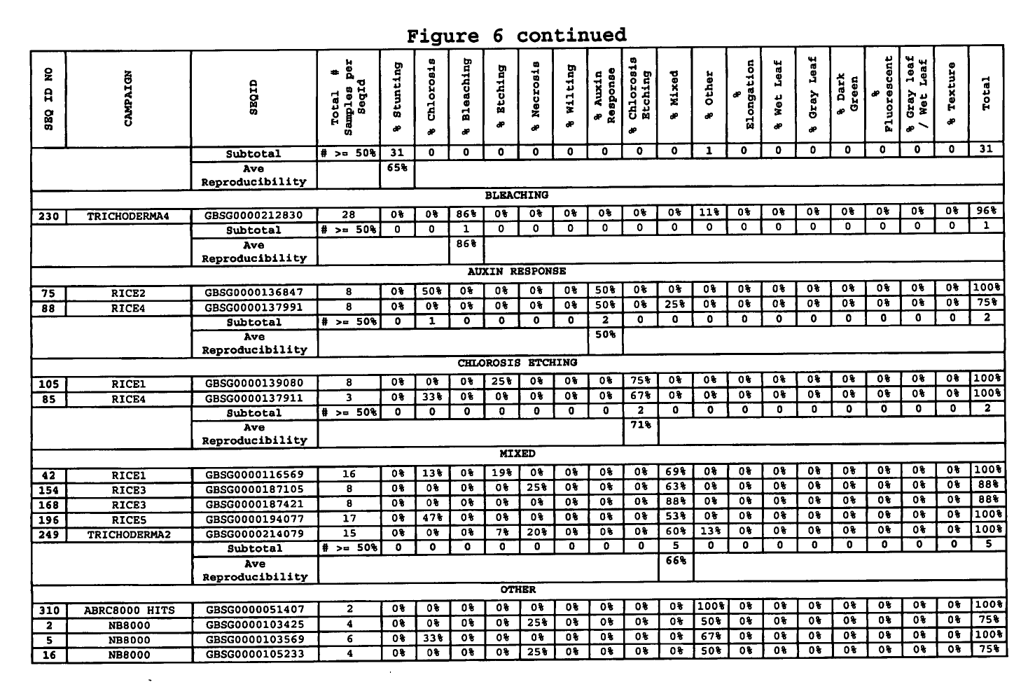

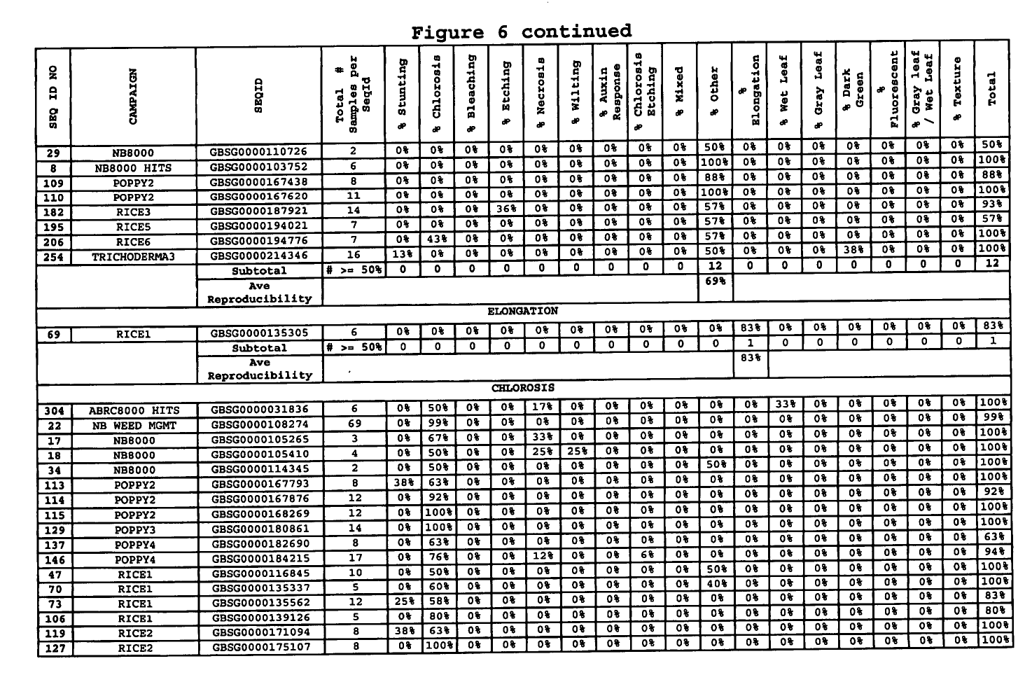

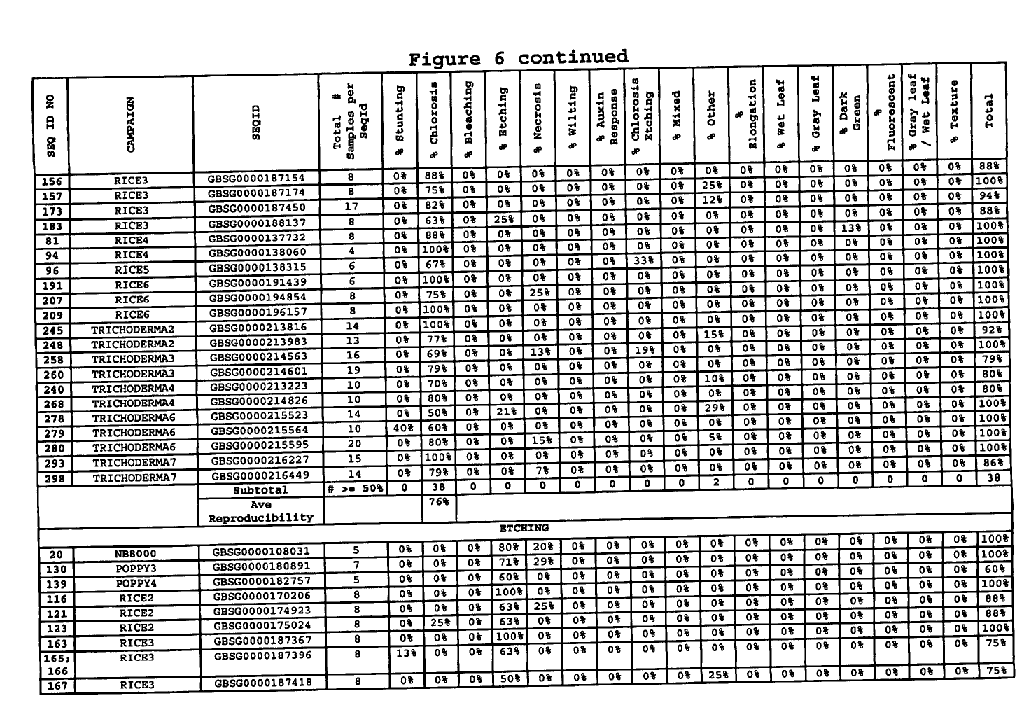

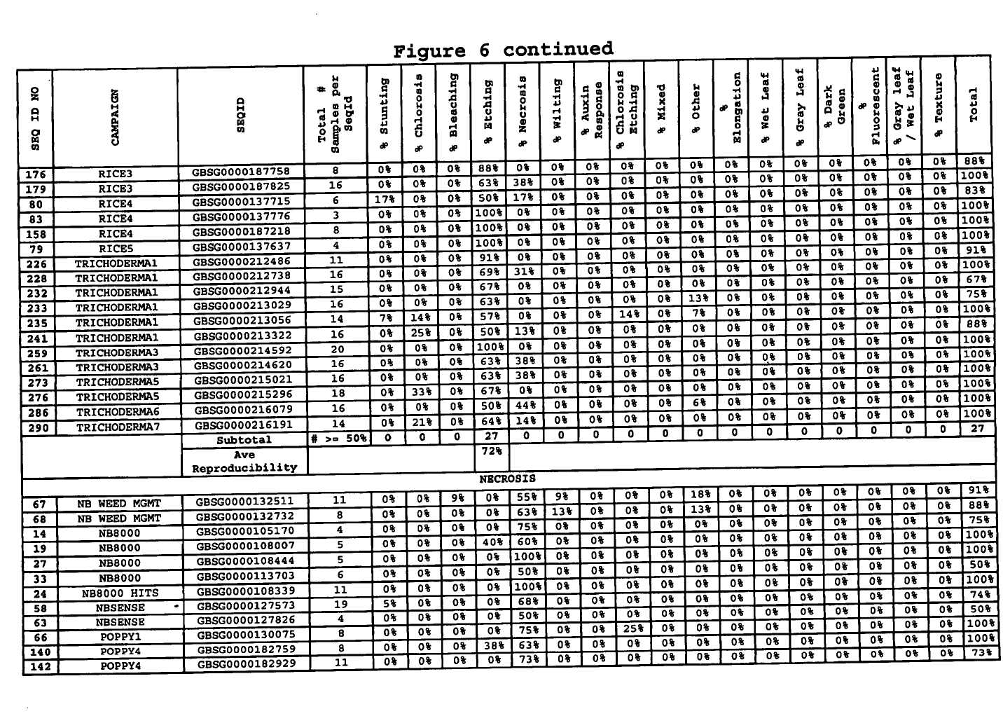

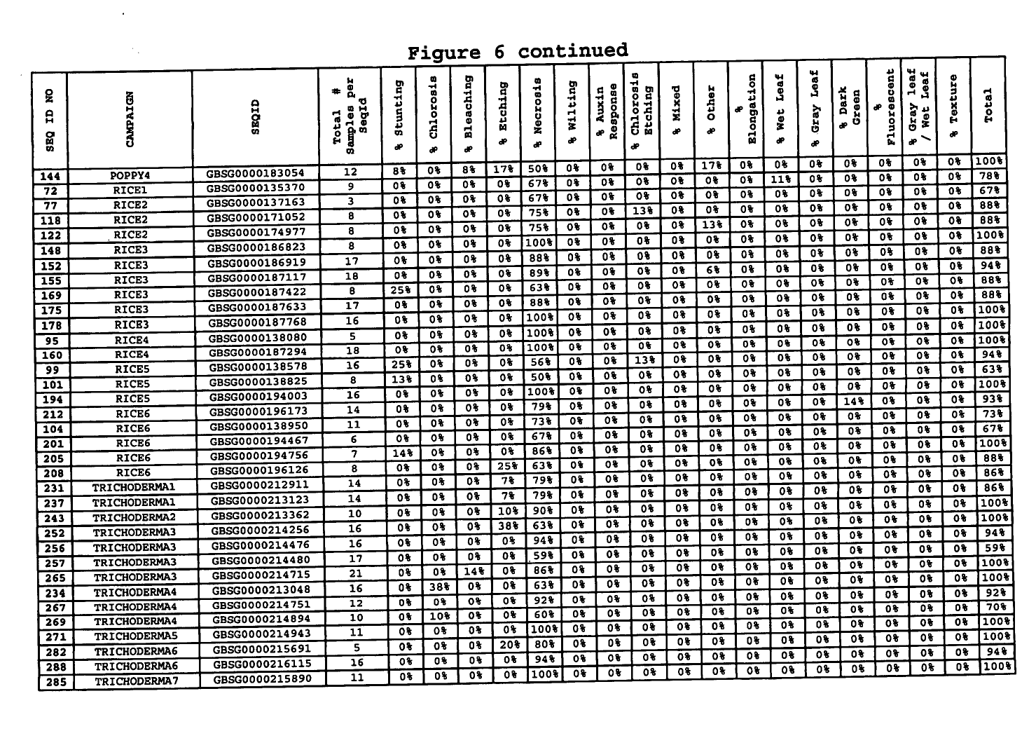

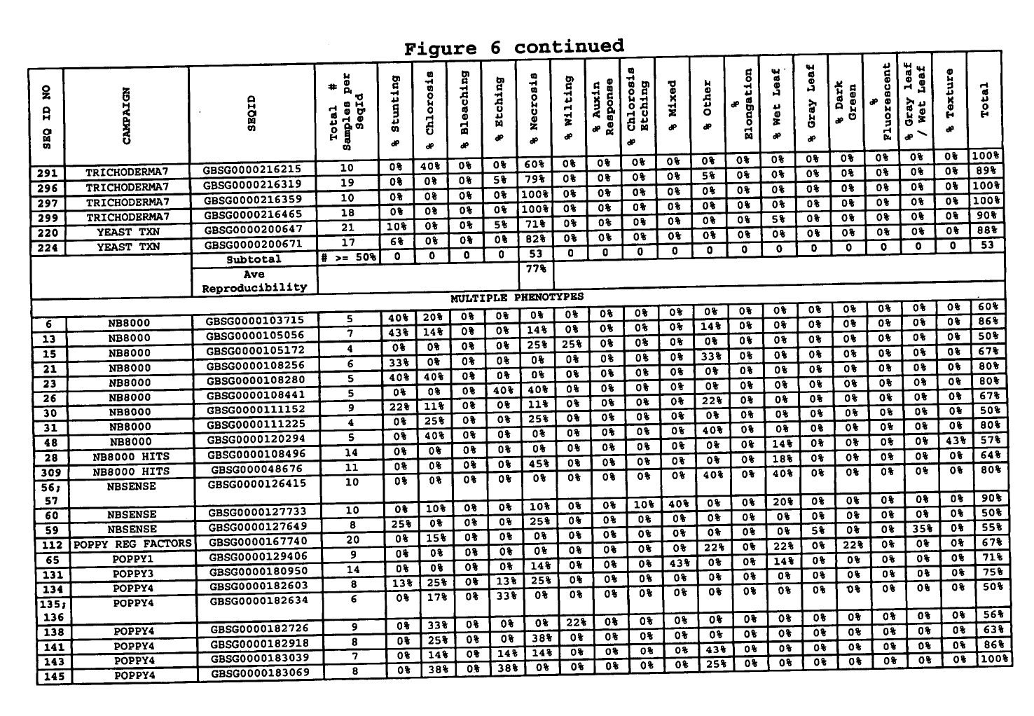

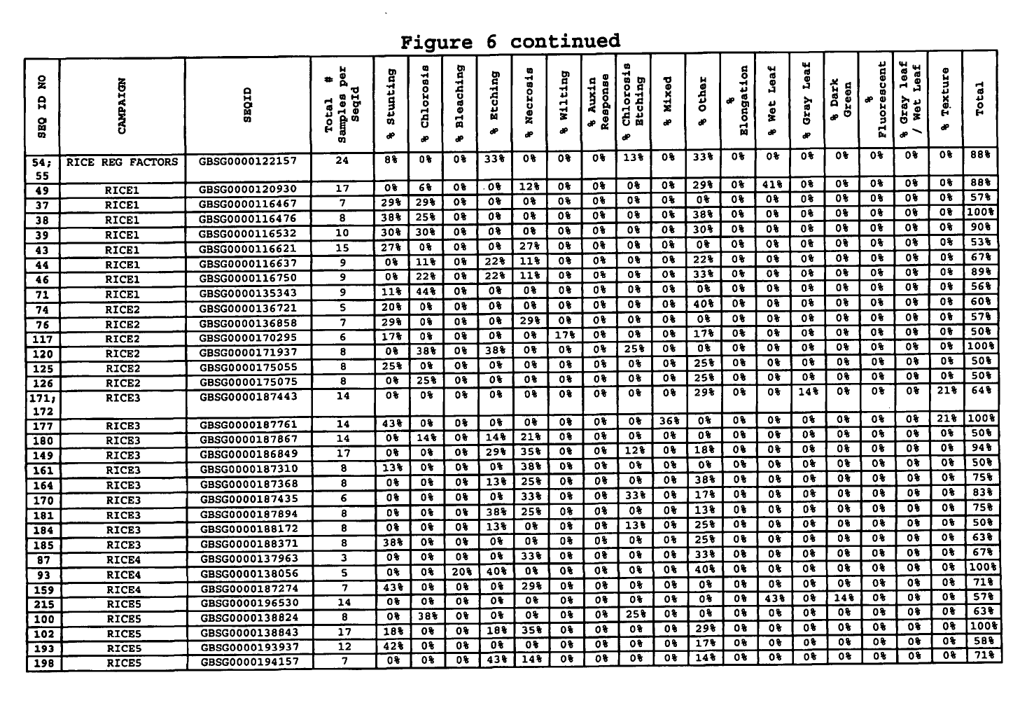

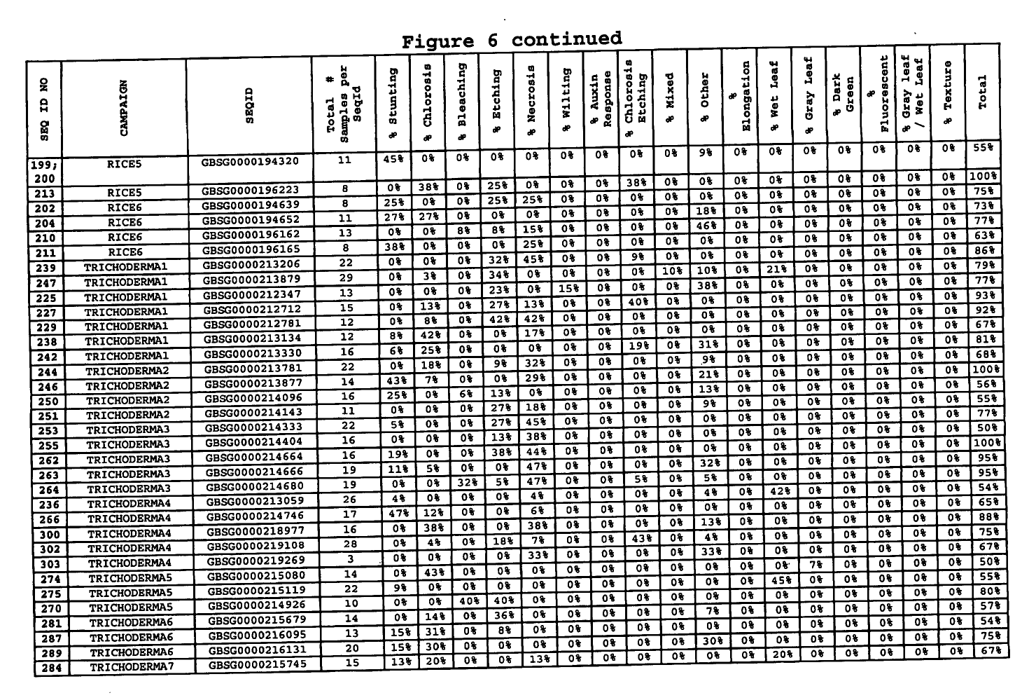

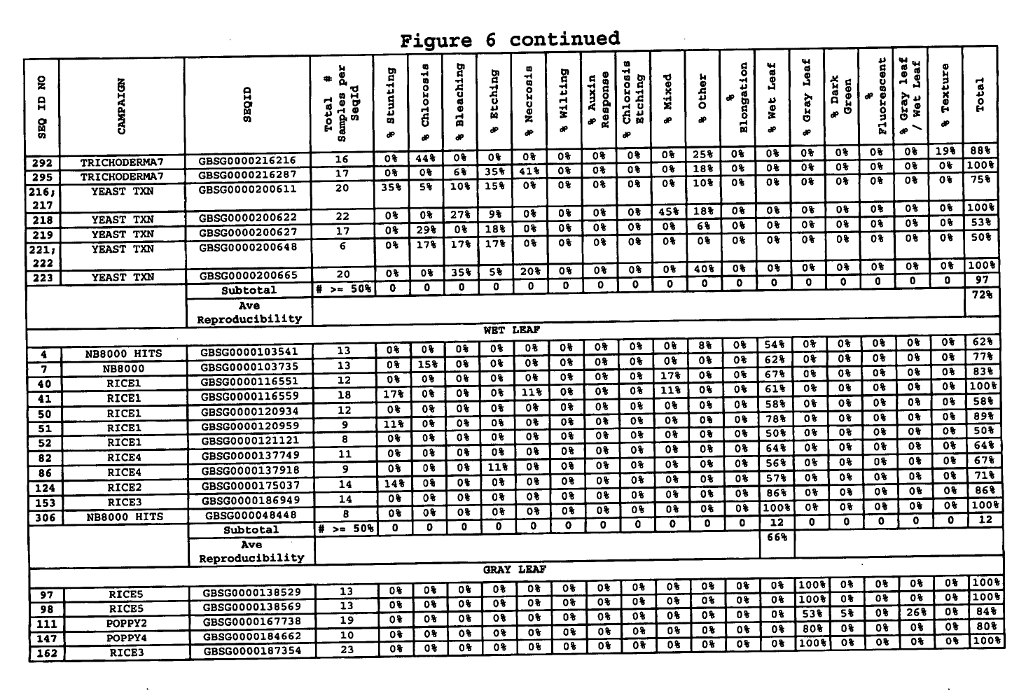

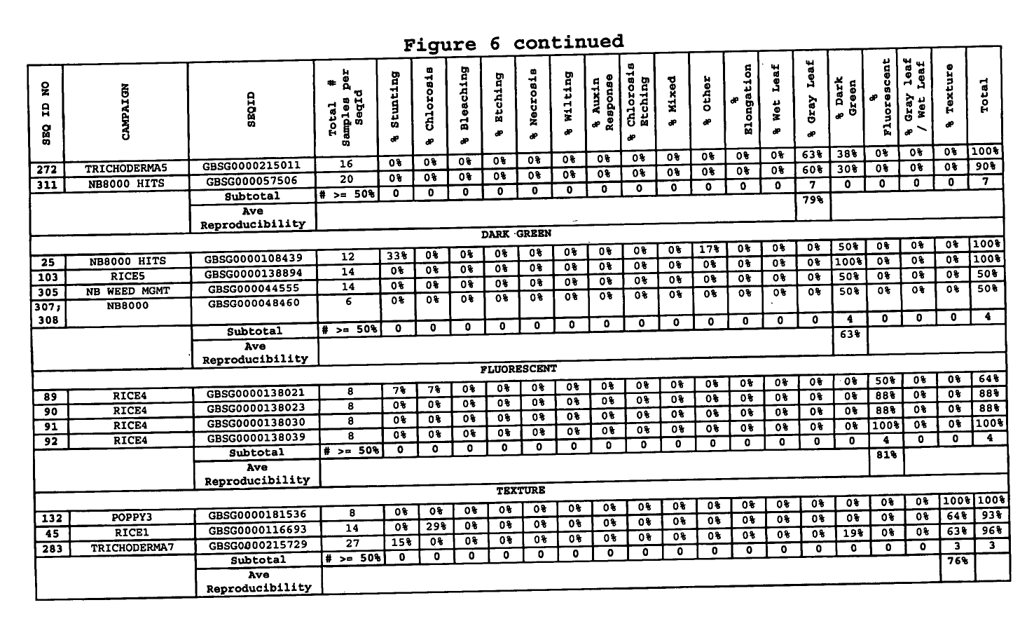

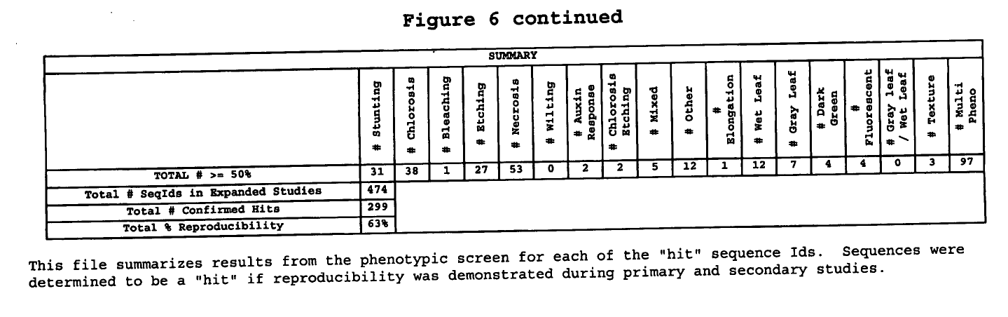

The present invention provides nucleic sequences involved in providing altered visual phenotypes in plants. Plants transformed with viral vectors comprising the nucleic acid sequences of the present invention were screened for an altered visual phenotype. The results are presented in FIG. 6. Accordingly, in some embodiments, the present invention provides nucleic acid sequences that produce an altered visual phenotype when expressed in plant (SEQ ID NOs:1-311 and 2024-2065, FIG. 1). The present invention is not limited to the particular nucleic acid sequences listed. Indeed, the present invention encompasses nucleic acid sequences (including sequences of the same, shorter, and longer lengths) that hybridize to the listed nucleic sequences under conditions ranging from low to high stringency and that also cause the altered visual phenotype. These sequences are conveniently identified by insertion into GENEWARE® vectors and expression in plants as detailed in the Examples.

In some embodiments, the sequences are operably linked to a plant promoter or provided in a vector as described in more detail above. These present invention also contemplates plants transformed or transfected with these sequences as well as seeds from such transfected plants. Furthermore, the sequences can expressed in either sense or antisense orientation. In particularly preferred embodiments, the sequences are at least 30 nucleotides in length up to the length of the full-length of the corresponding gene. It is contemplated that sequences of less than full length (for example, greater than about 30 nucleotides) are useful for down regulation of gene expression via antisense or cosupression. Suitable sequences are selected by chemically synthesizing the sequences, cloning into GENEWARE® expression vectors, expressing in plants, and selecting plants with an altered visual phenotype.

### VII. Identification of Homologs to Sequences

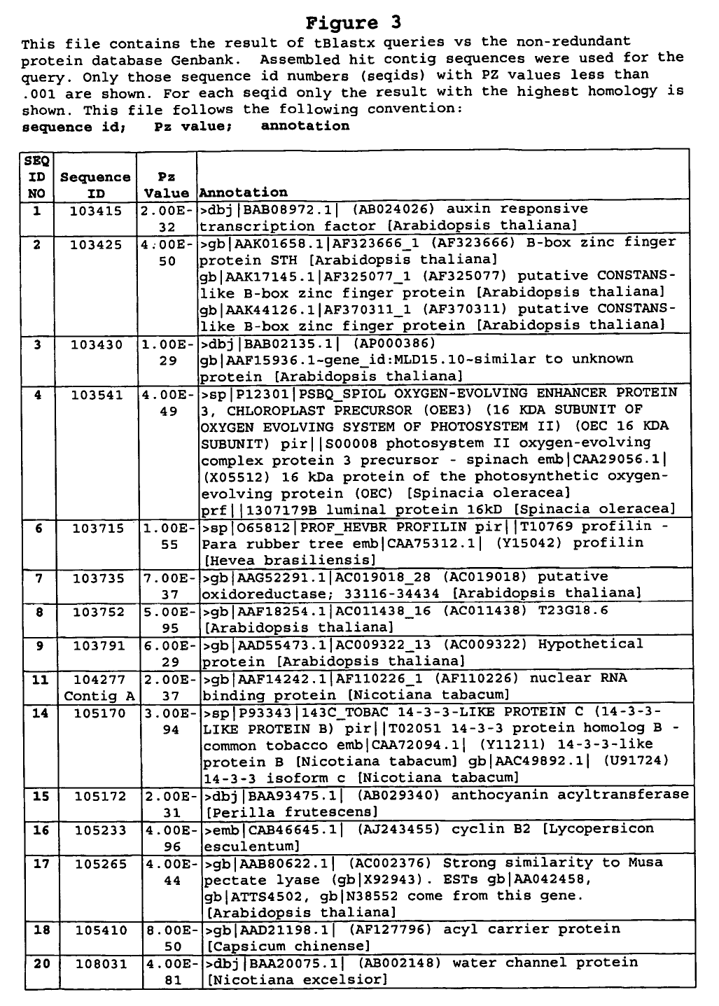

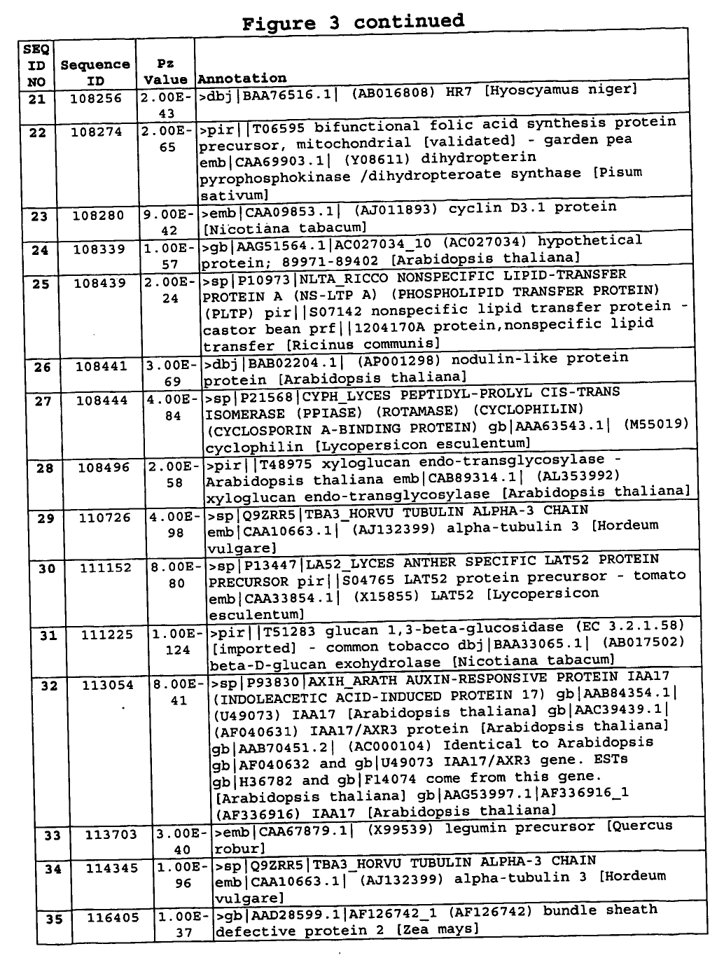

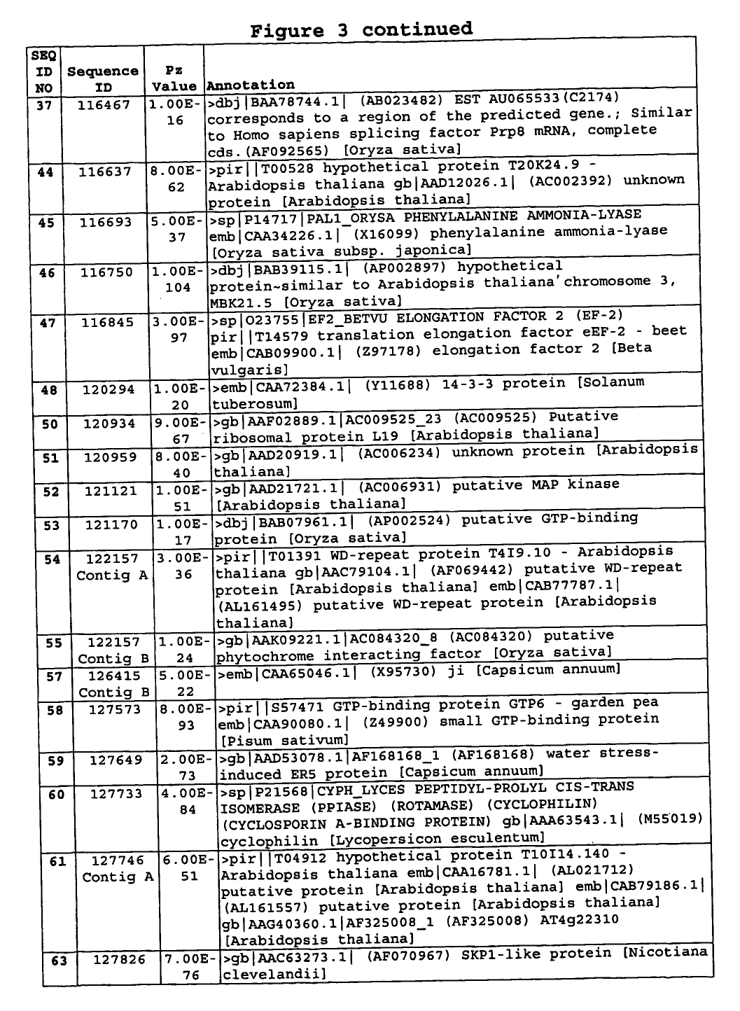

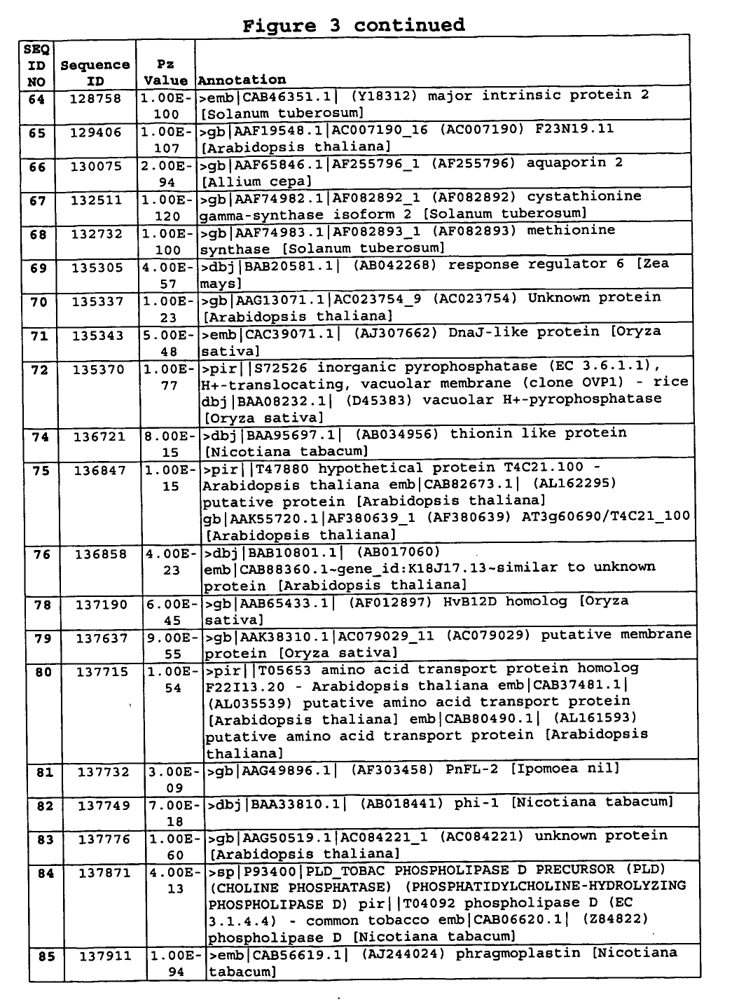

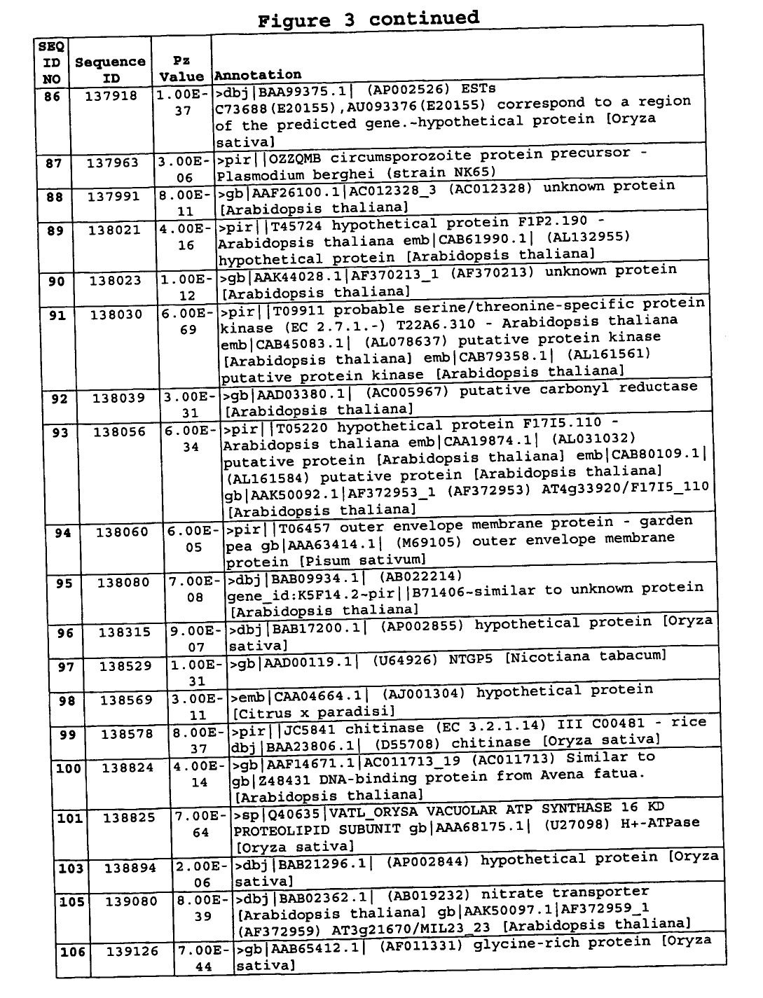

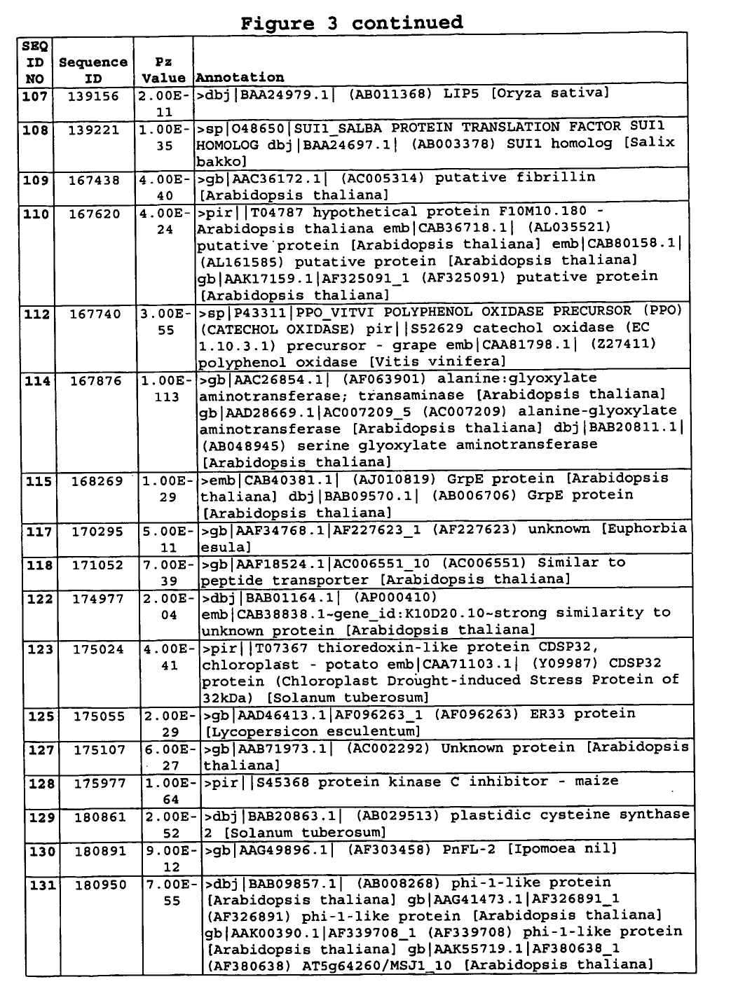

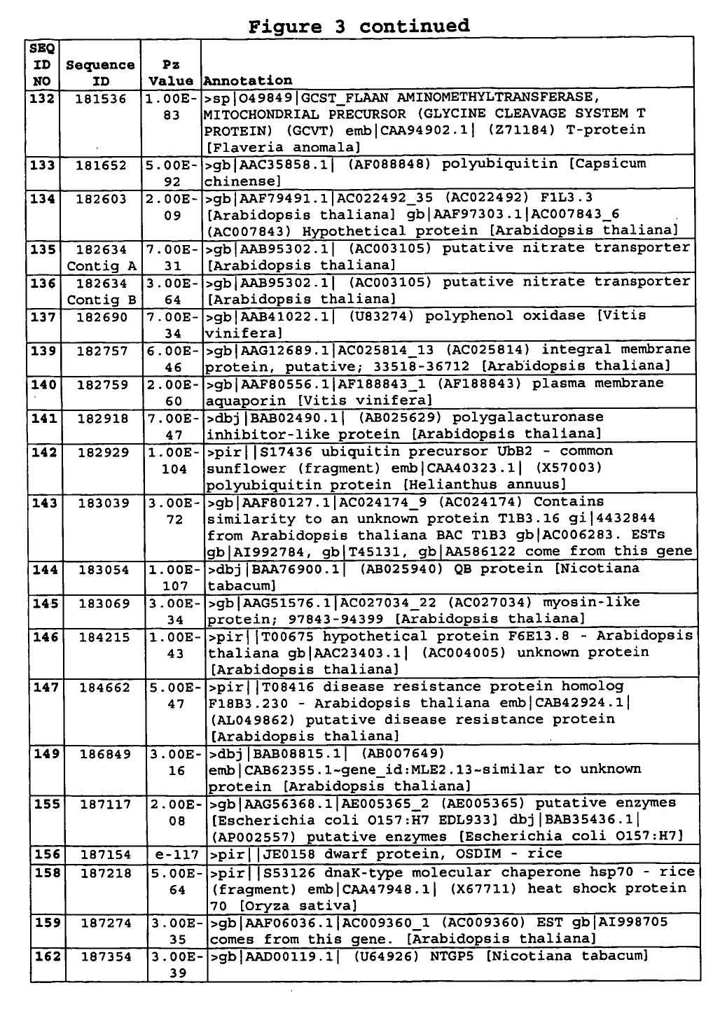

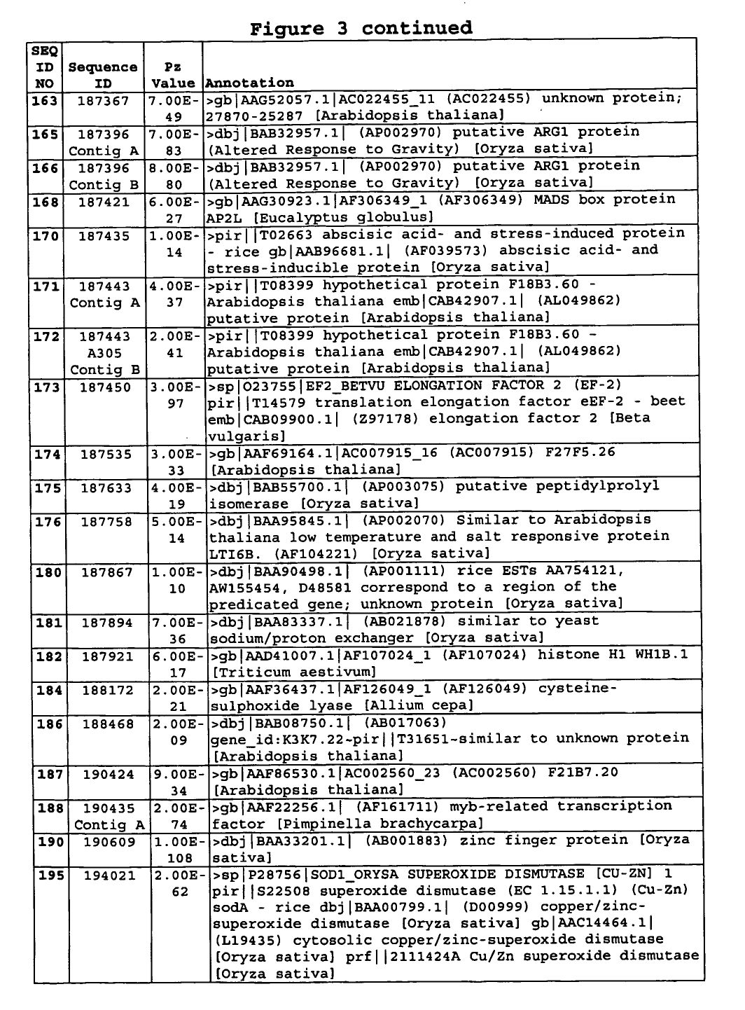

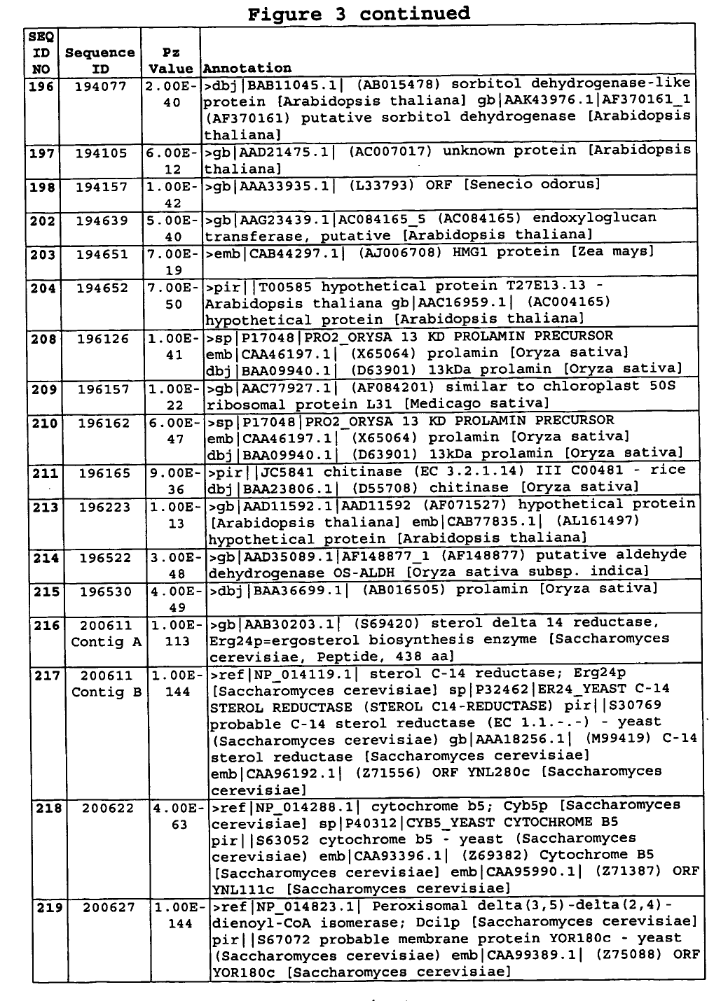

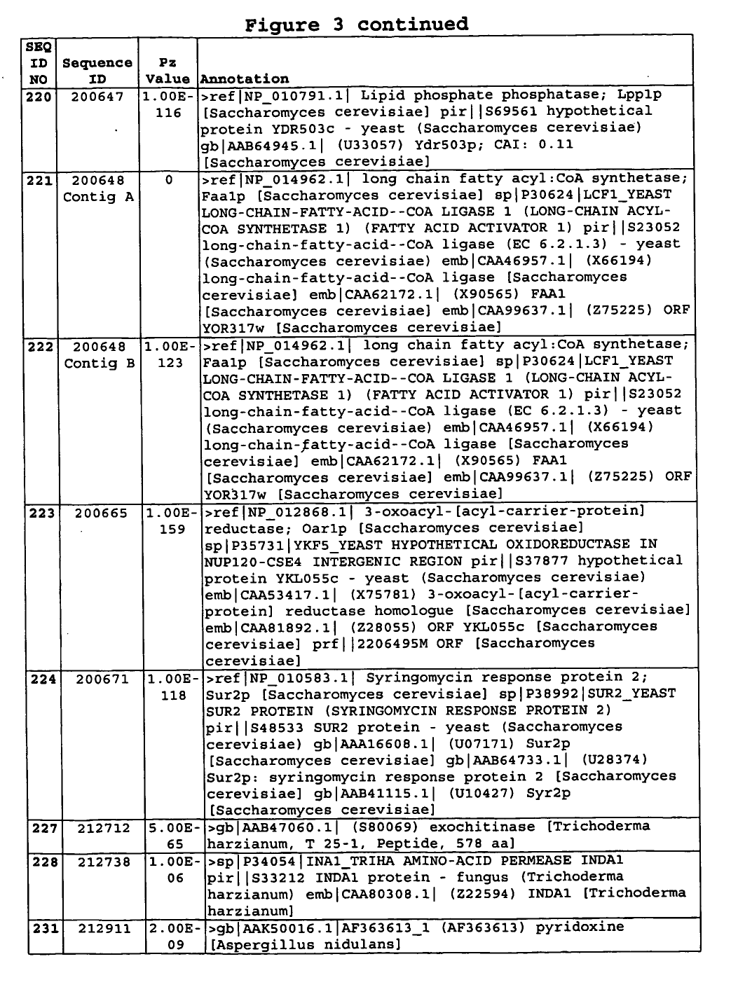

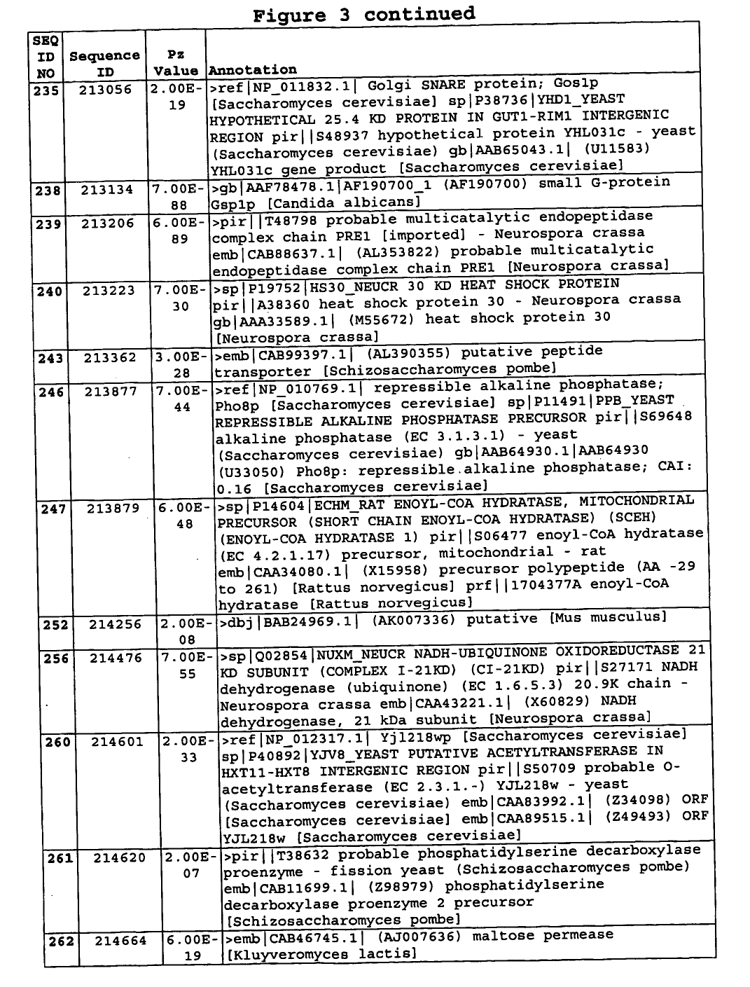

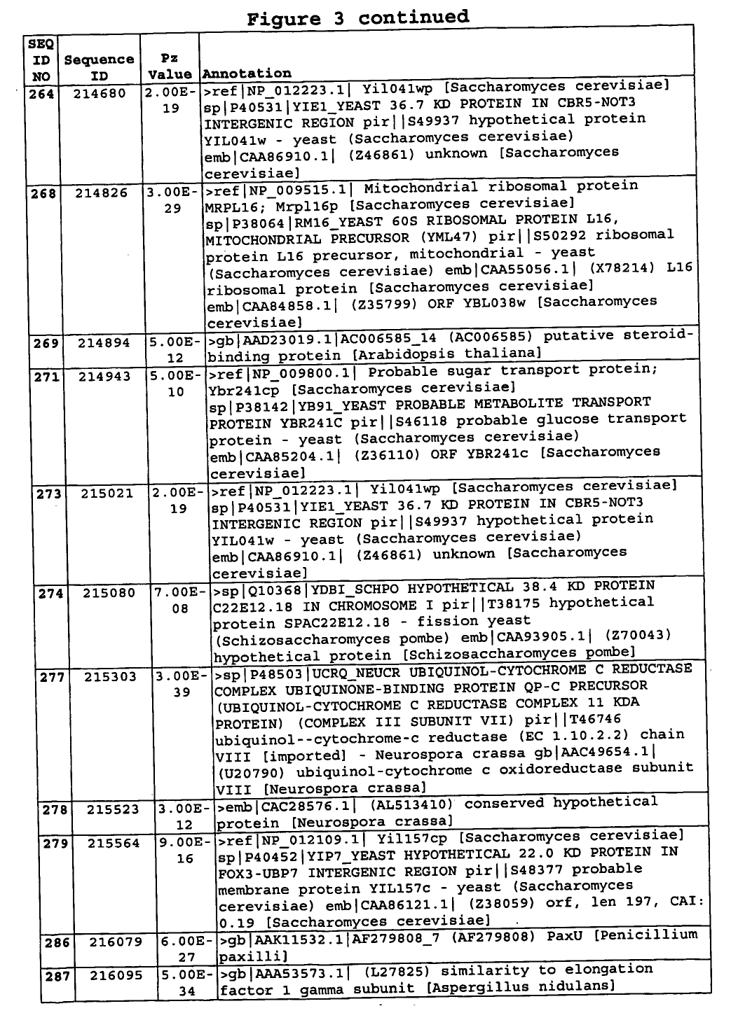

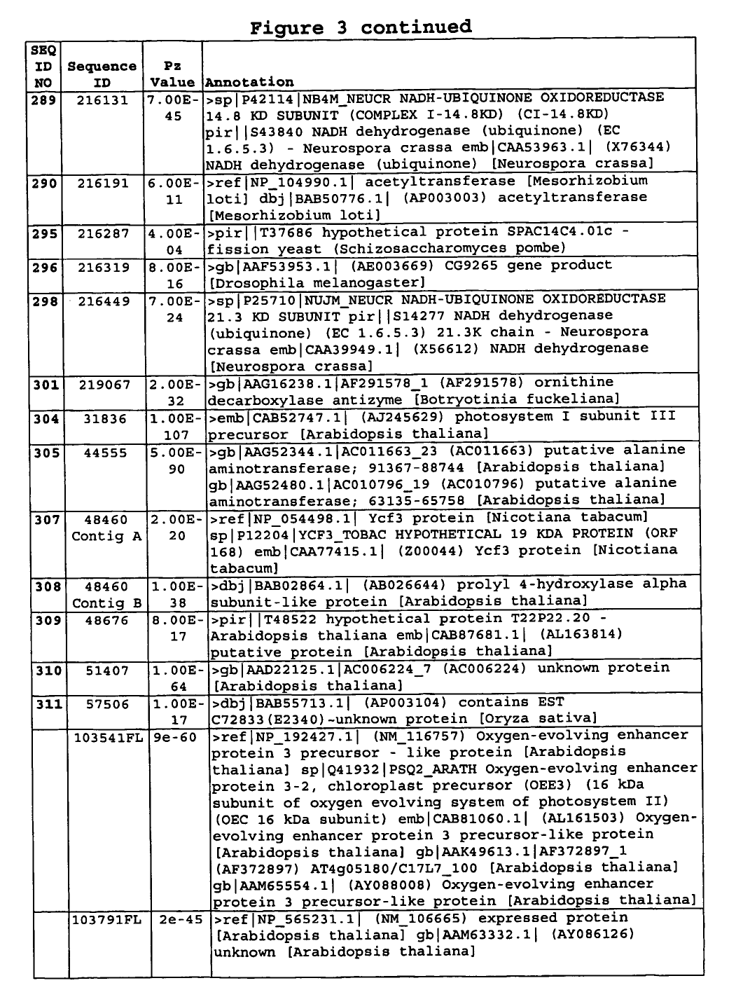

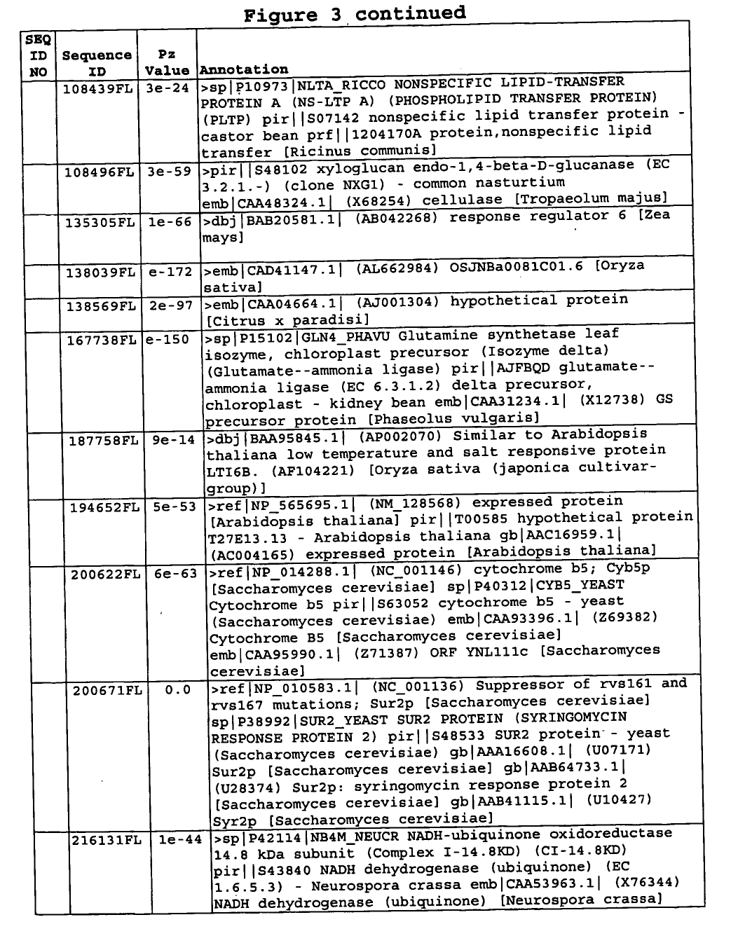

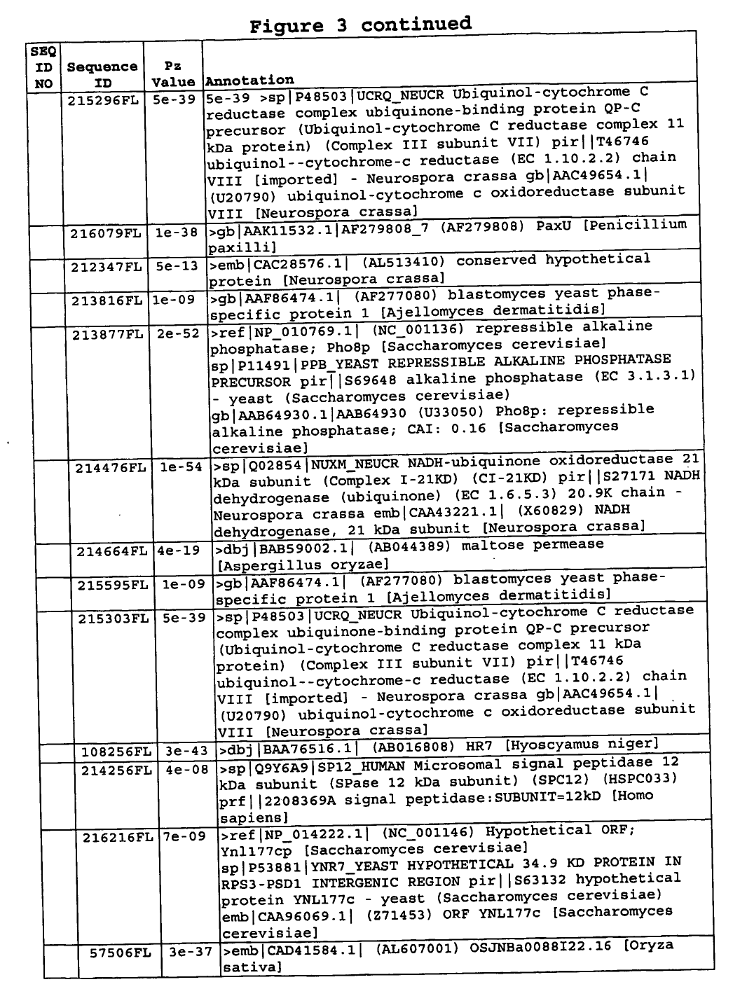

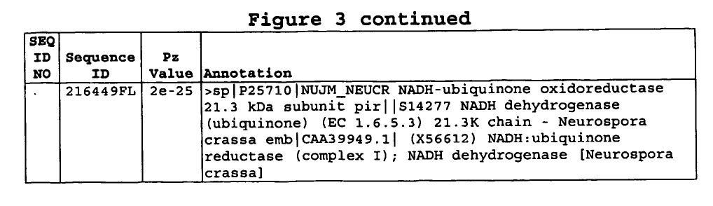

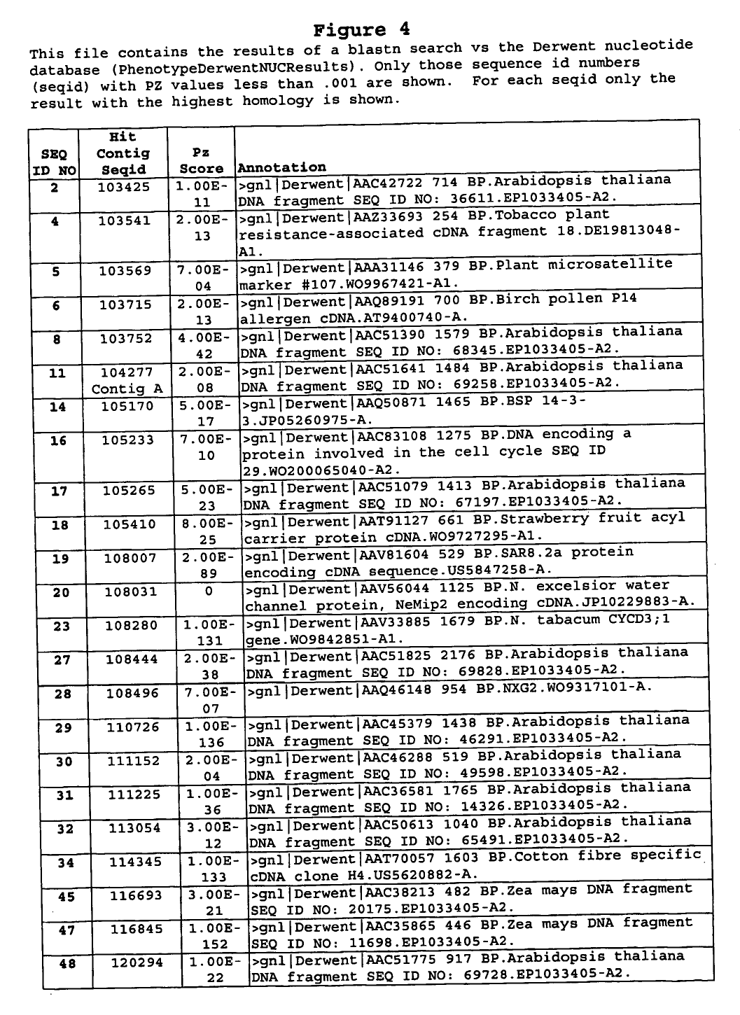

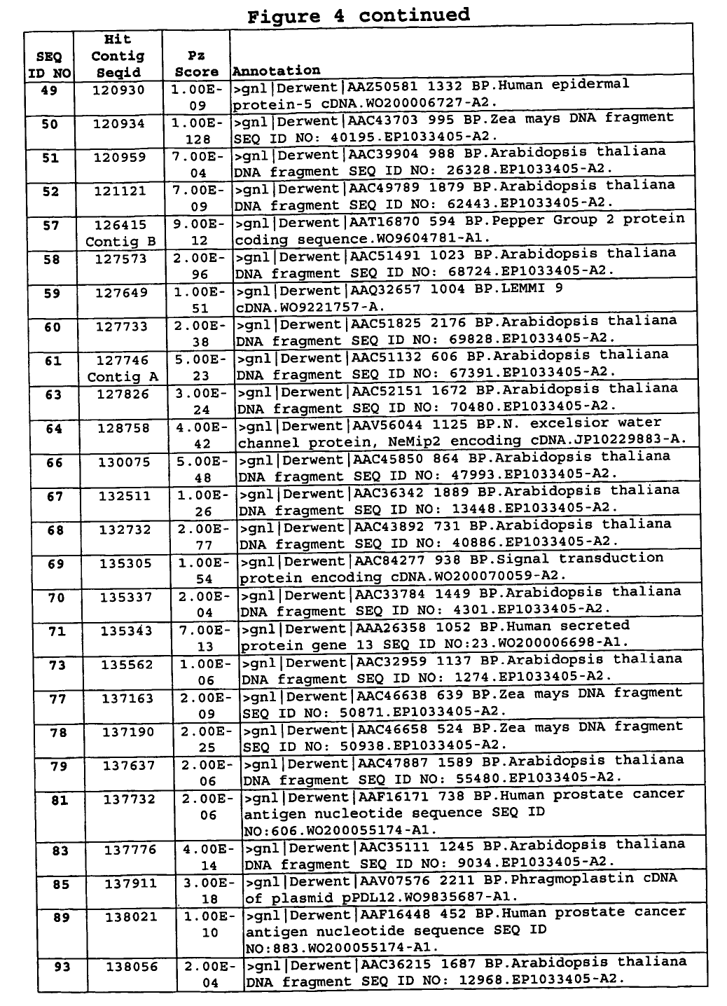

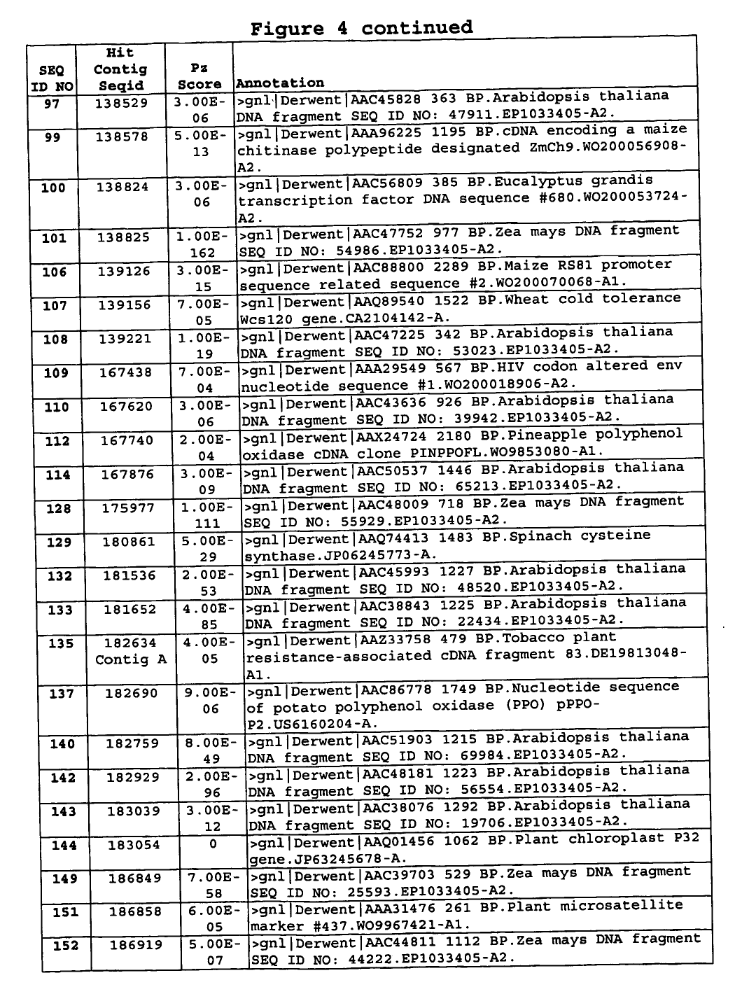

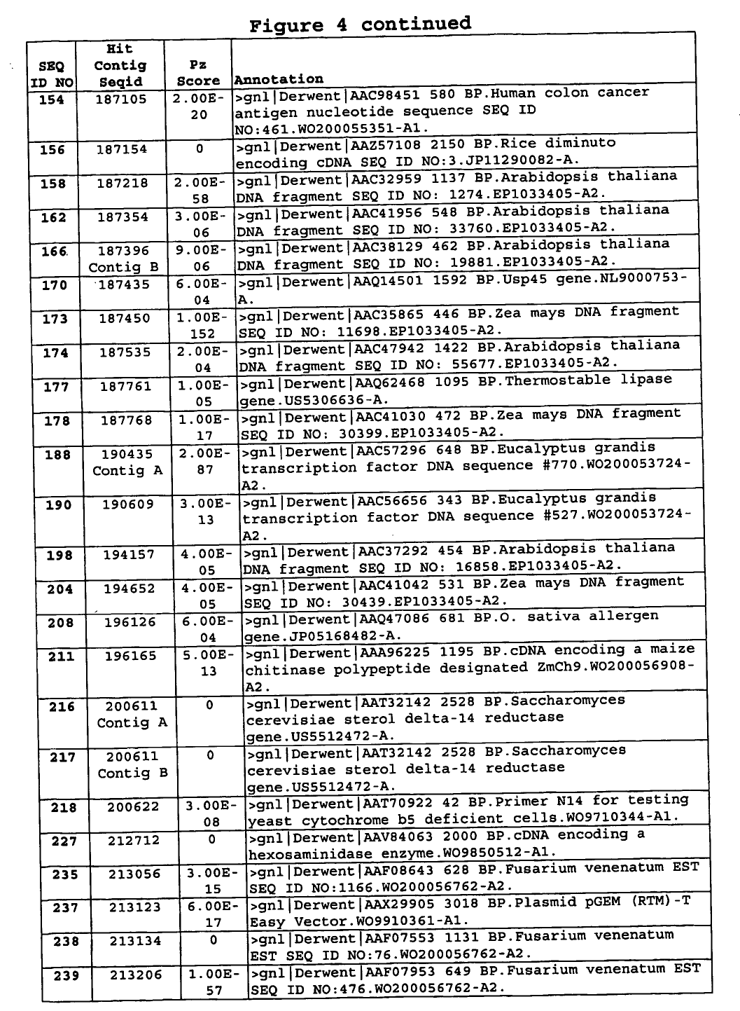

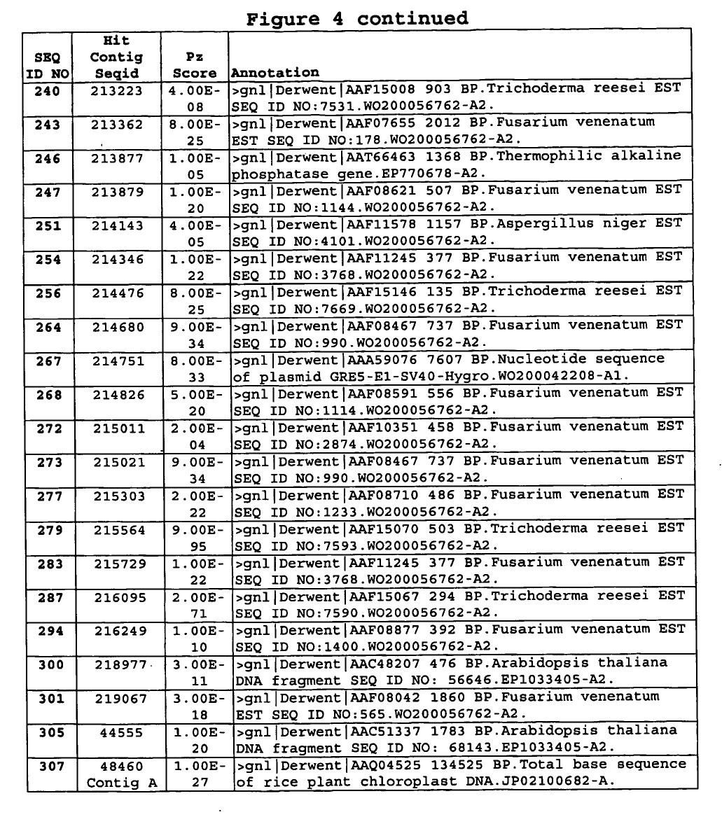

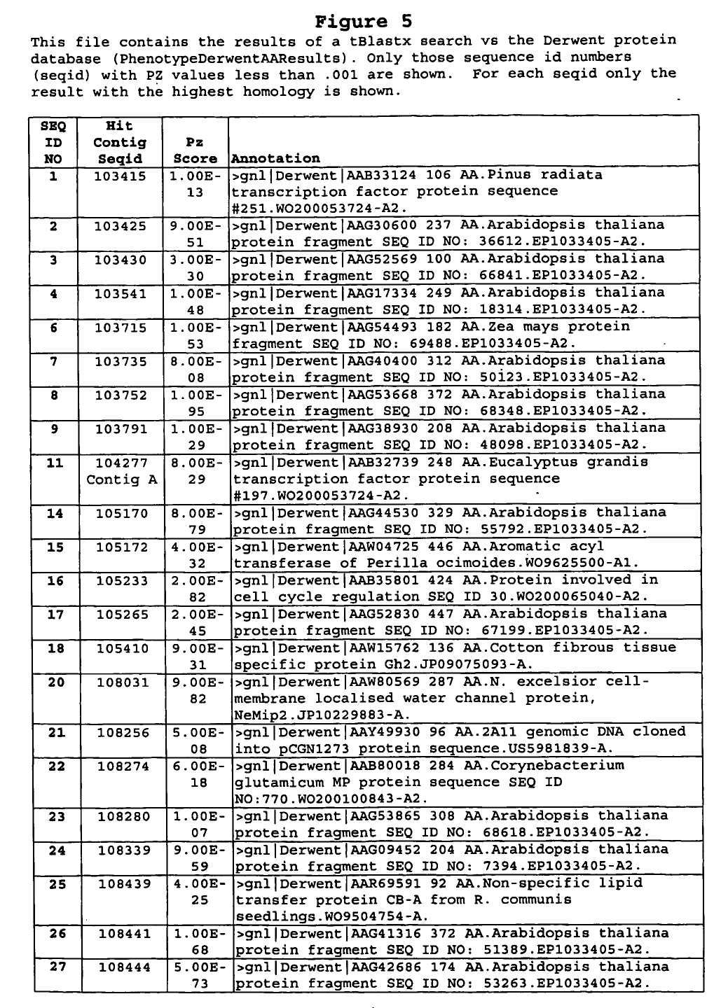

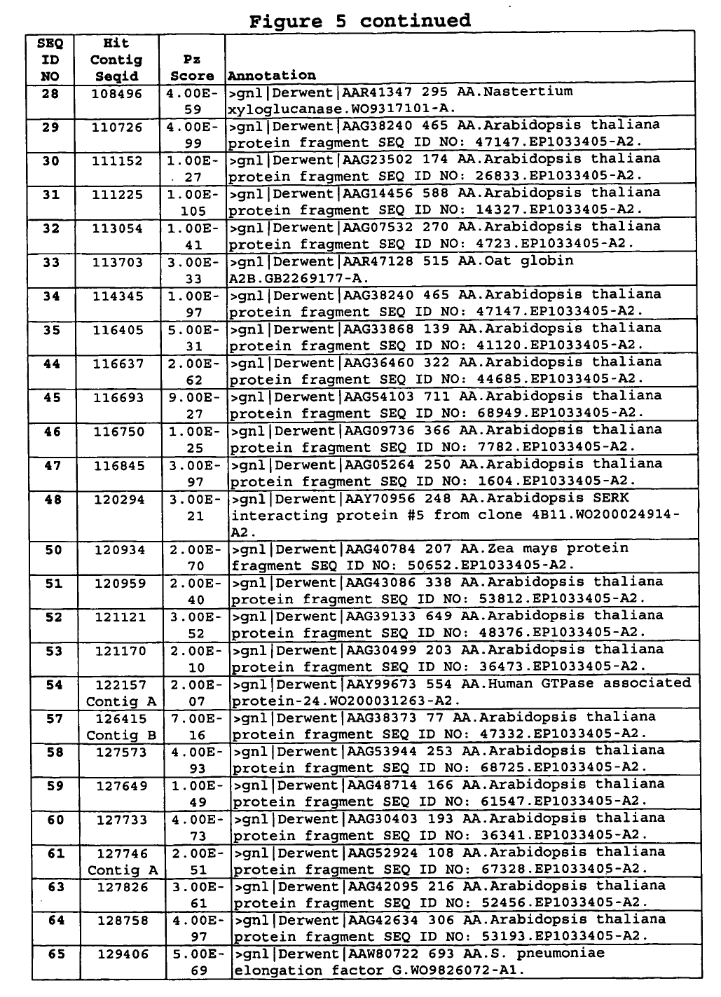

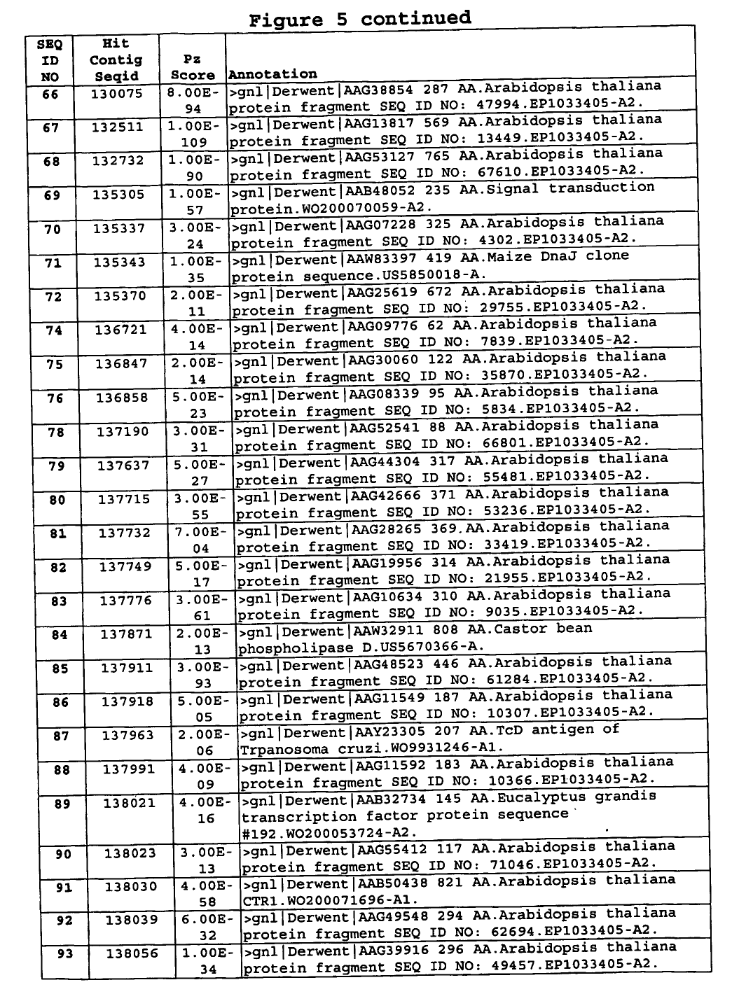

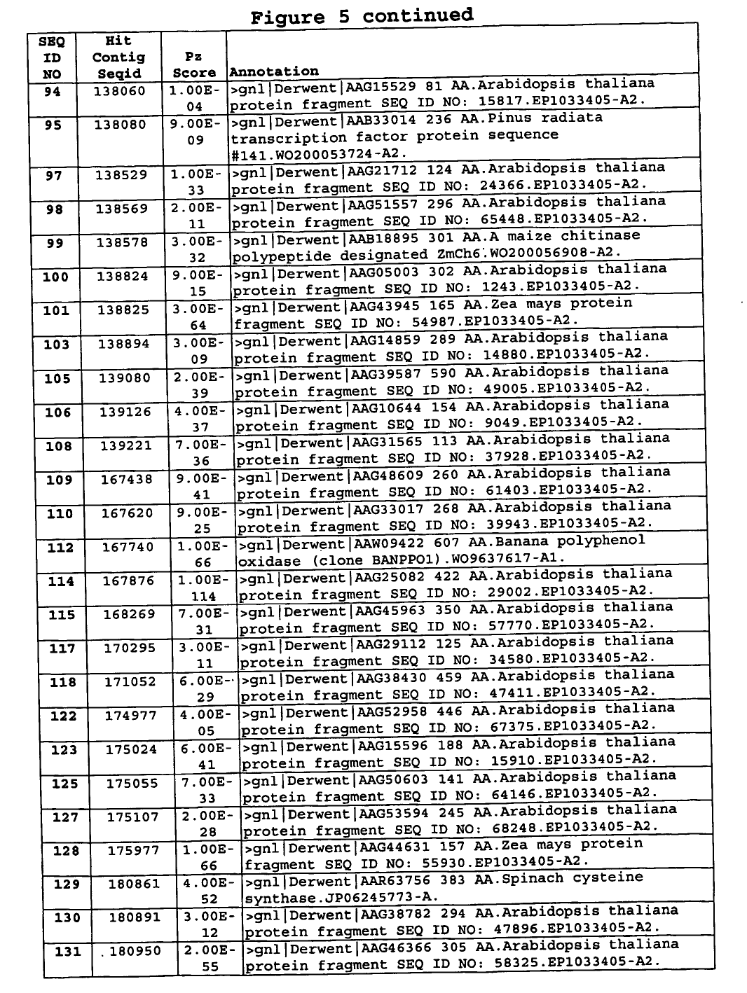

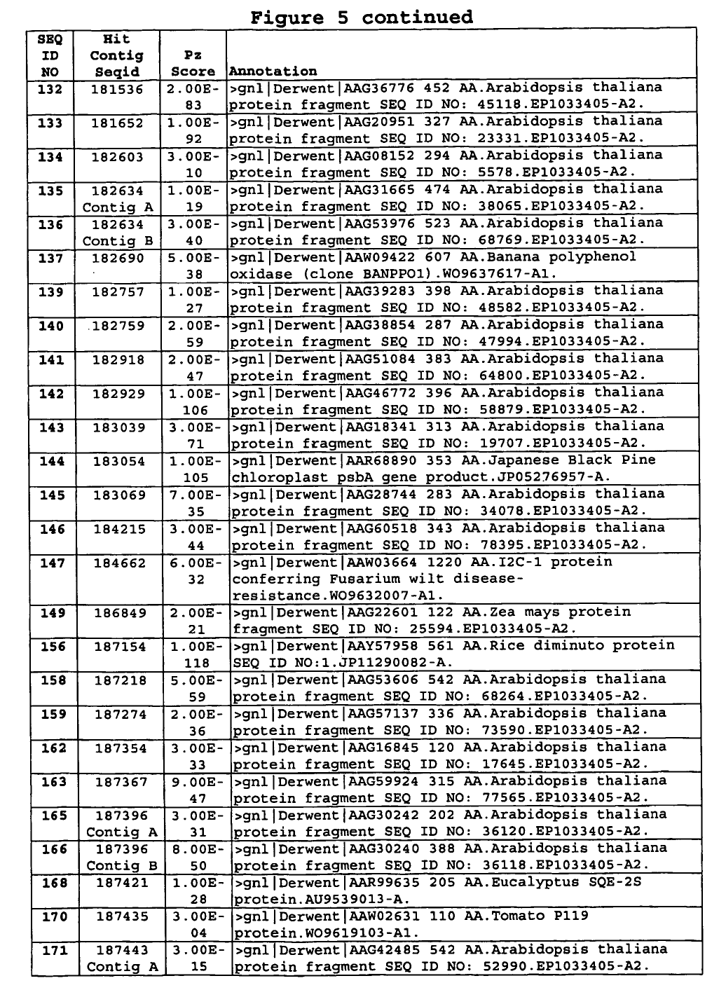

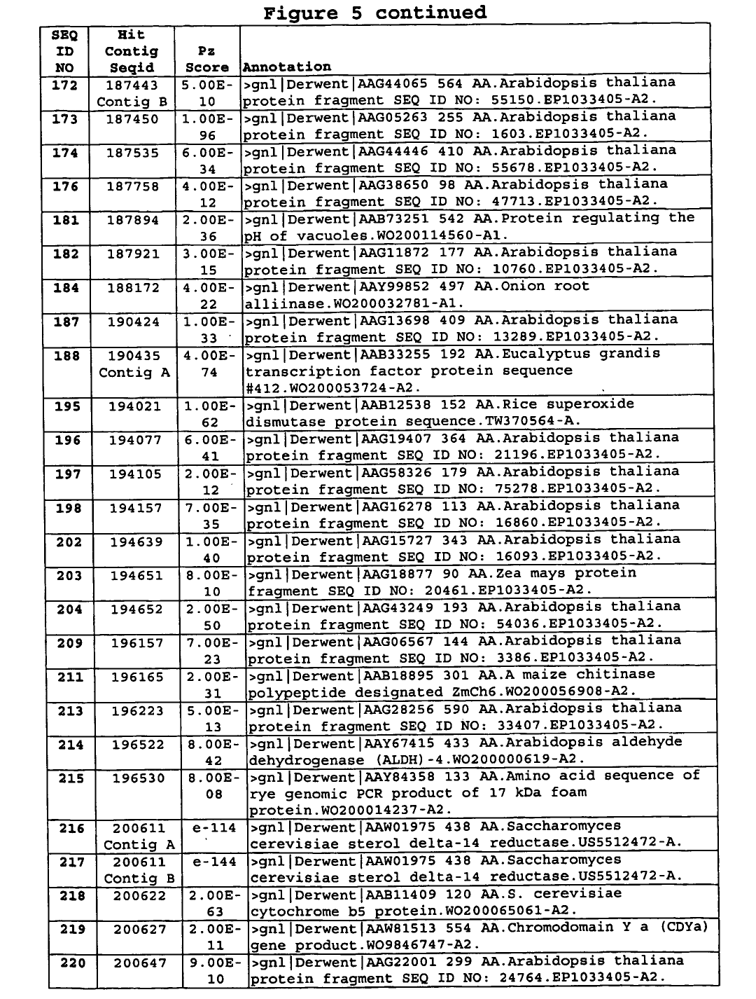

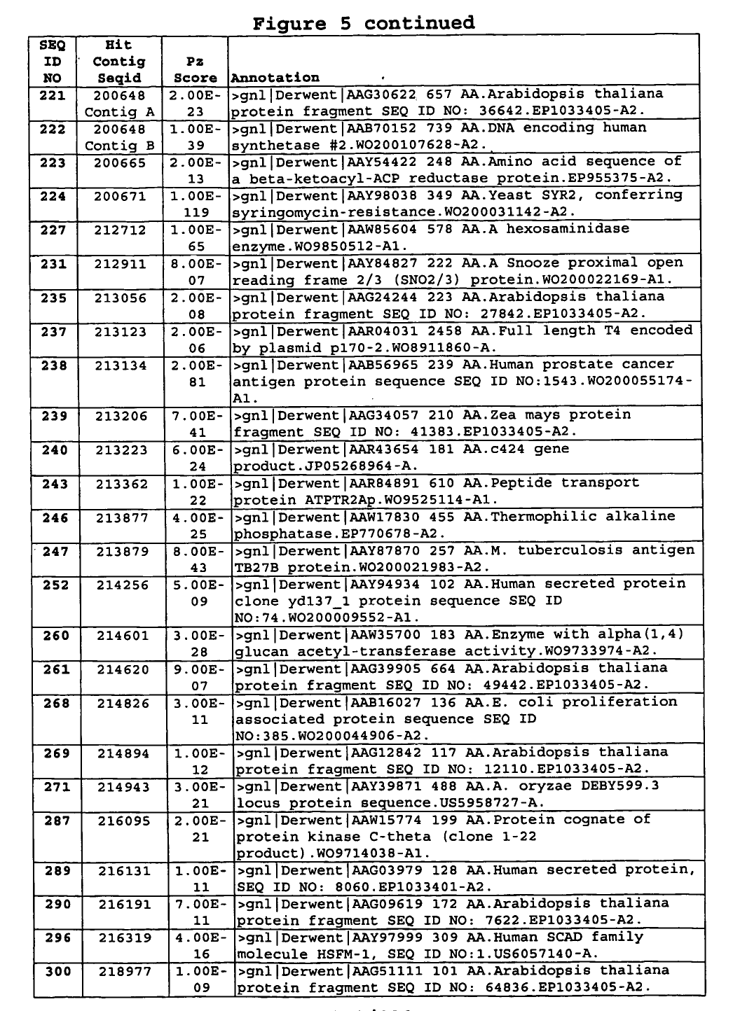

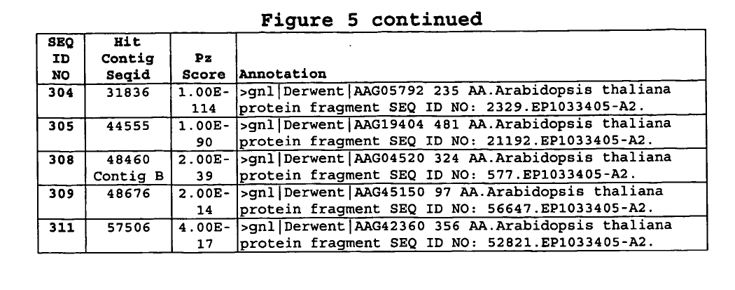

The present invention also provides homologs and variants of the sequences described above, but which may not hybridize to the sequences described above under conditions ranging from low to high stringency. In some preferred embodiments, the homologous and variant sequences are operably linked to an exogenous promoter. FIG. 3 provides BLASTX search results from publicly available databases. The relevant sequences are identified by Accession number in these databases. FIG. 4 contains the top blastx hits (identified by accession number) versus all the amino acid sequences in the Derwent biweekly database. FIG. 5 contains the top blastn hits (identified by accession number) versus all the nucleotide sequences in the Derwent biweekly database.

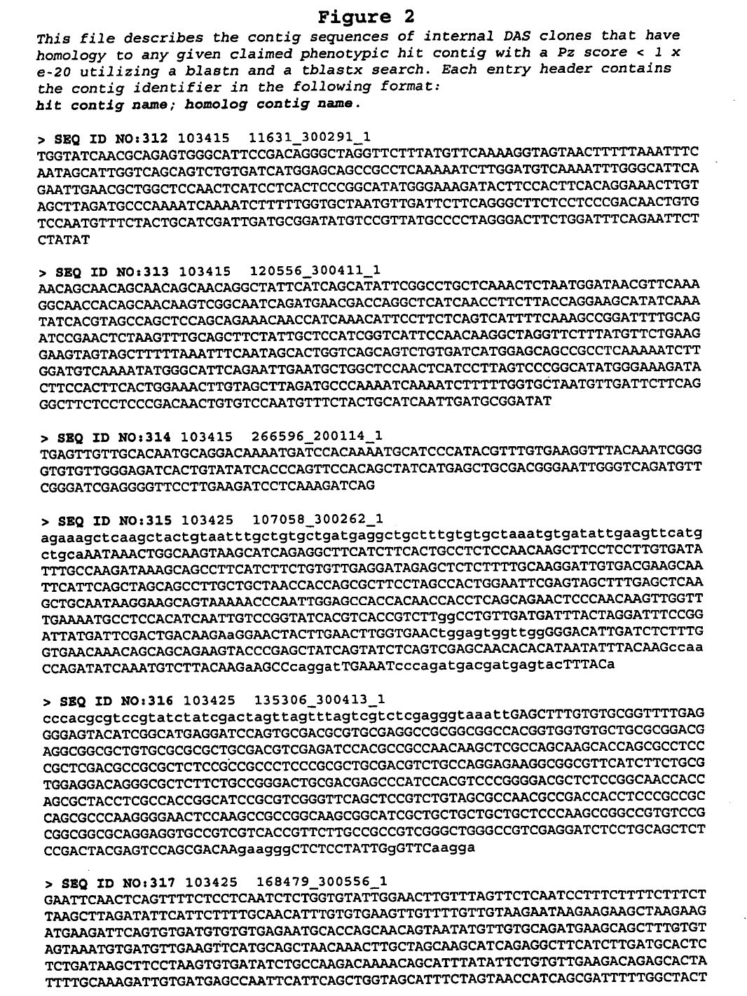

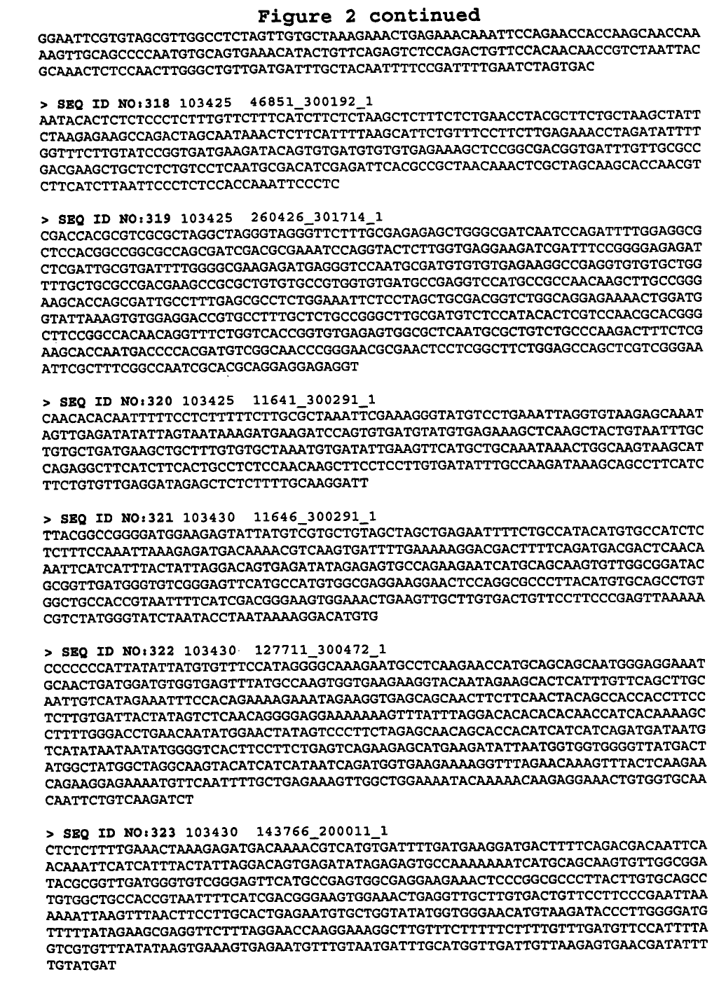

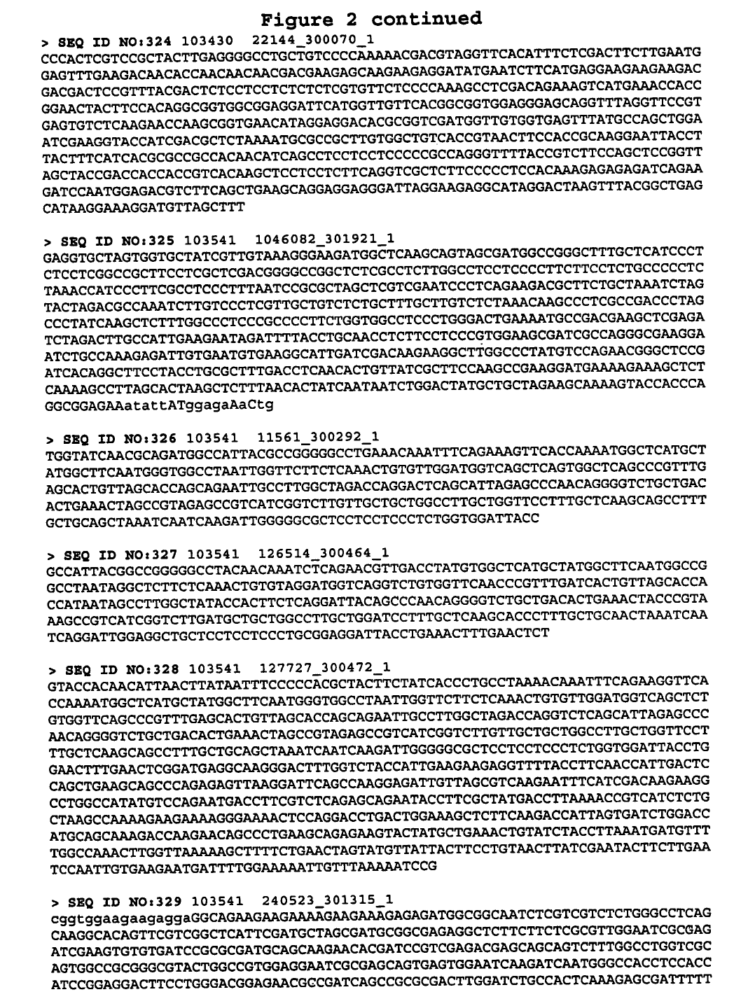

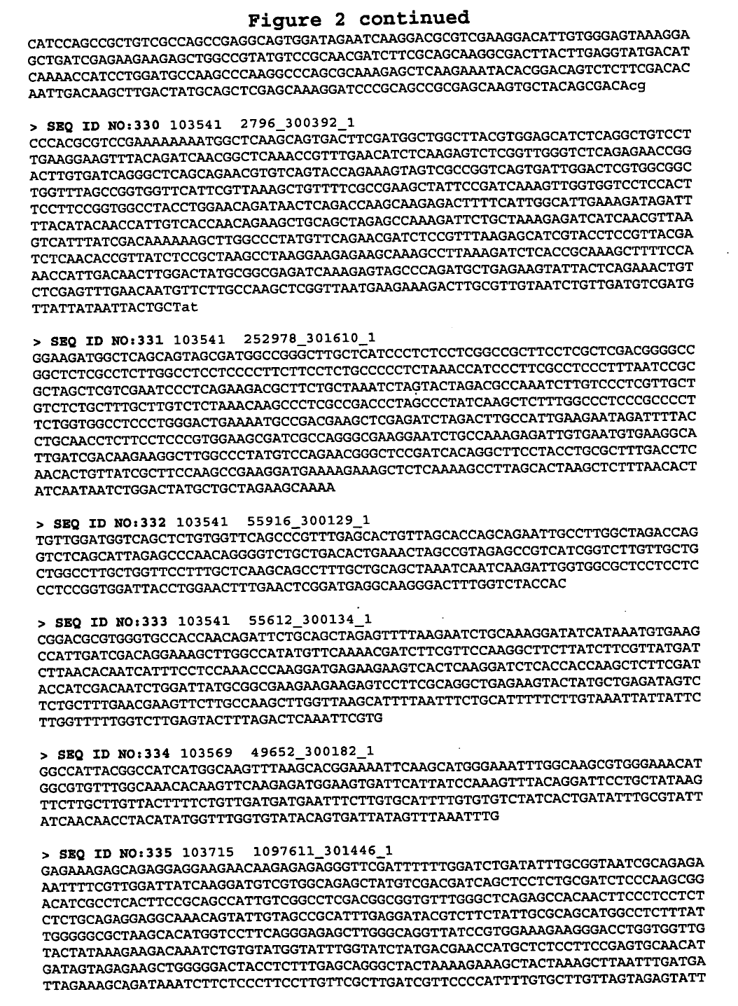

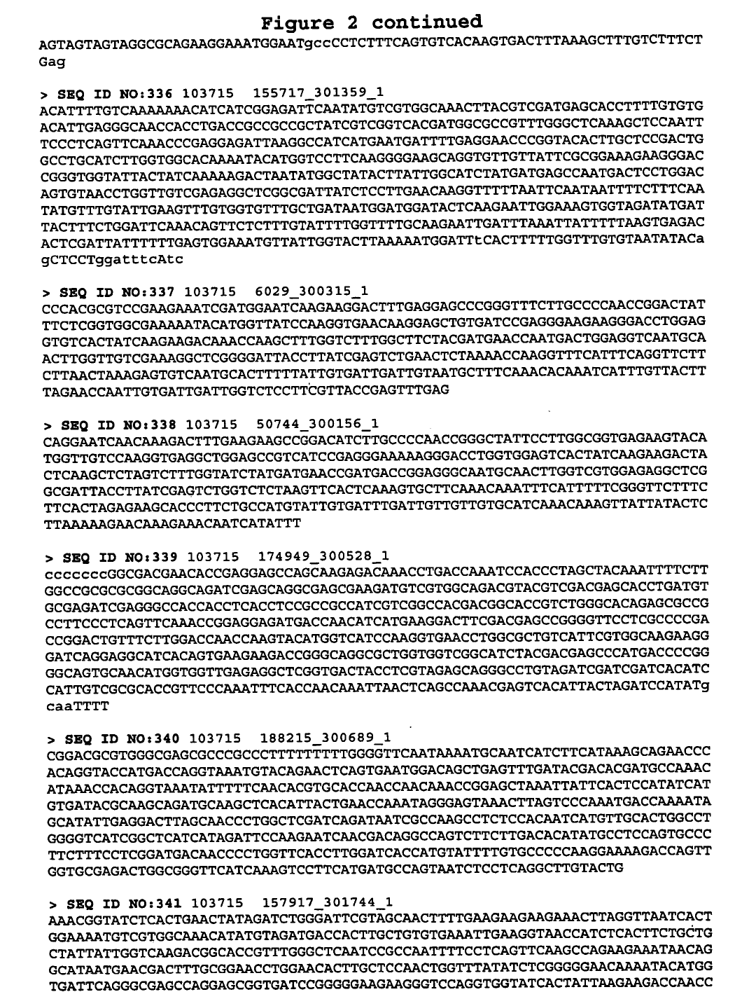

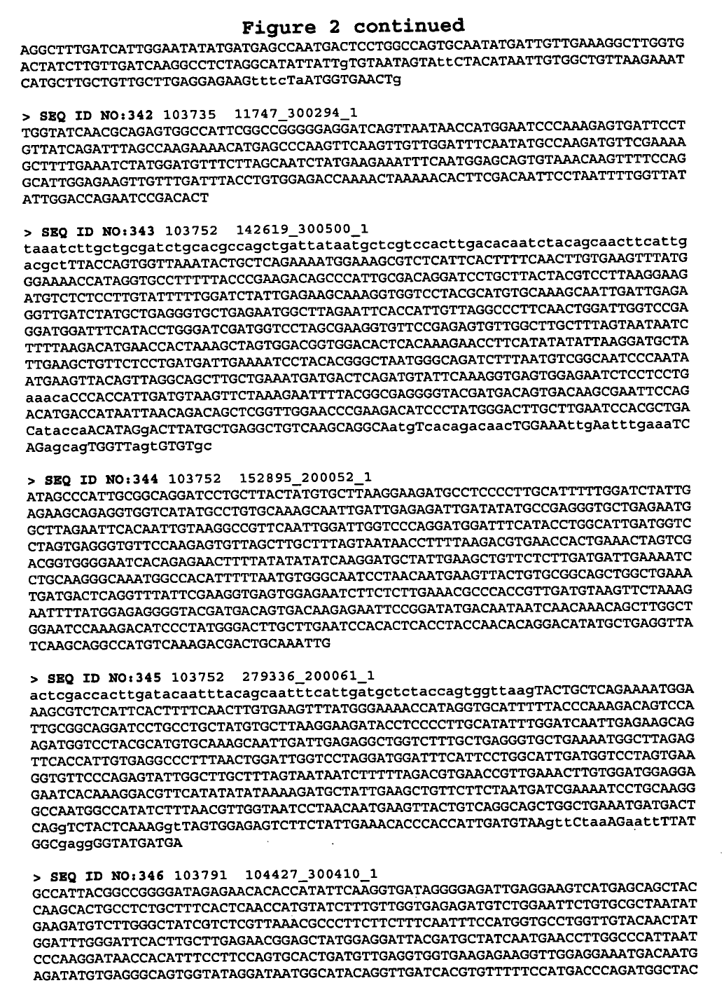

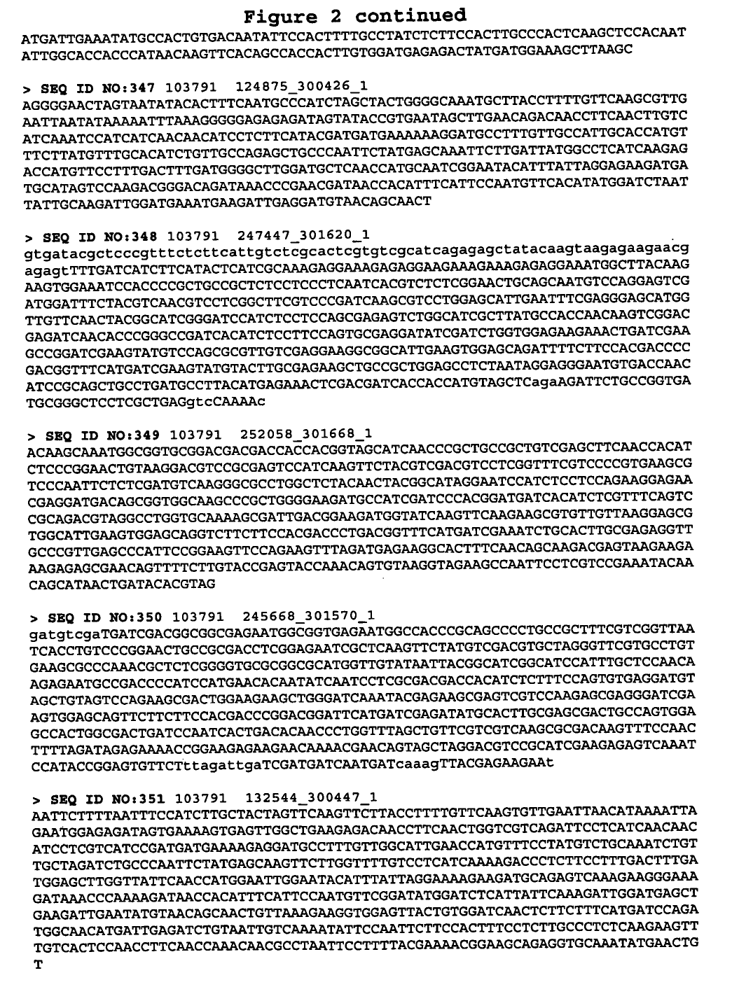

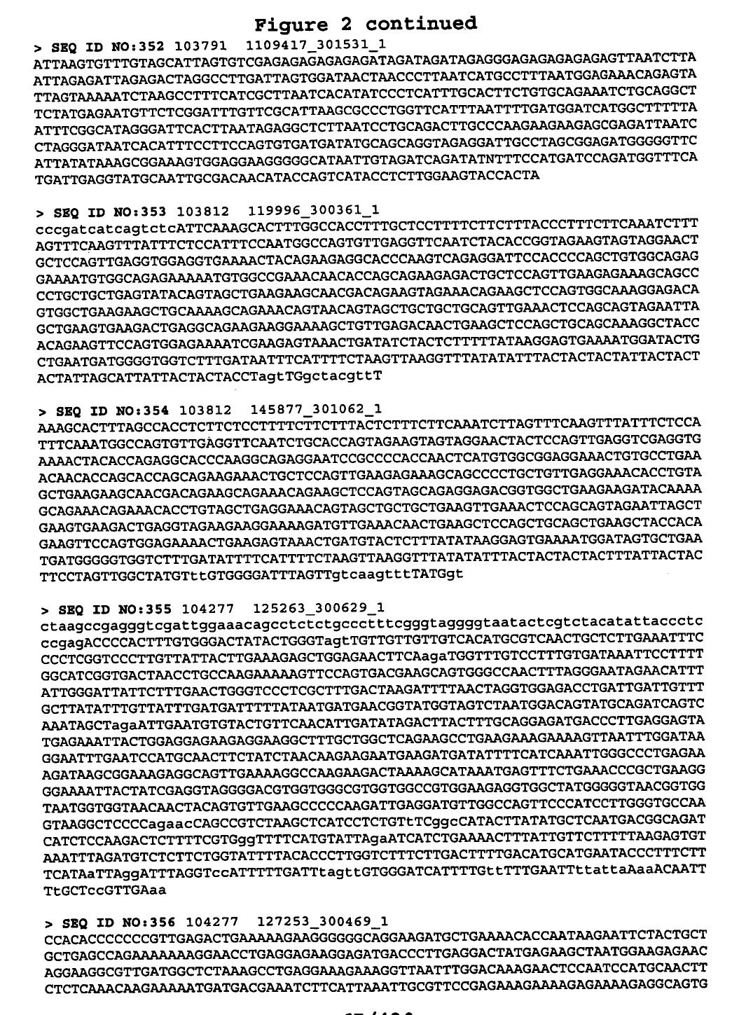

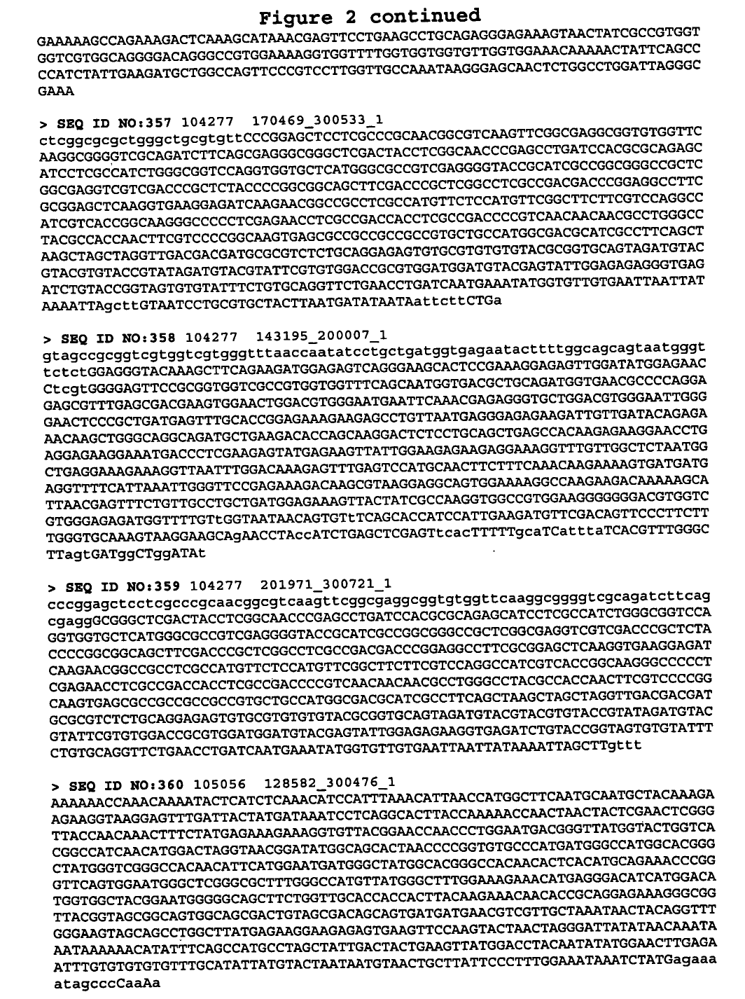

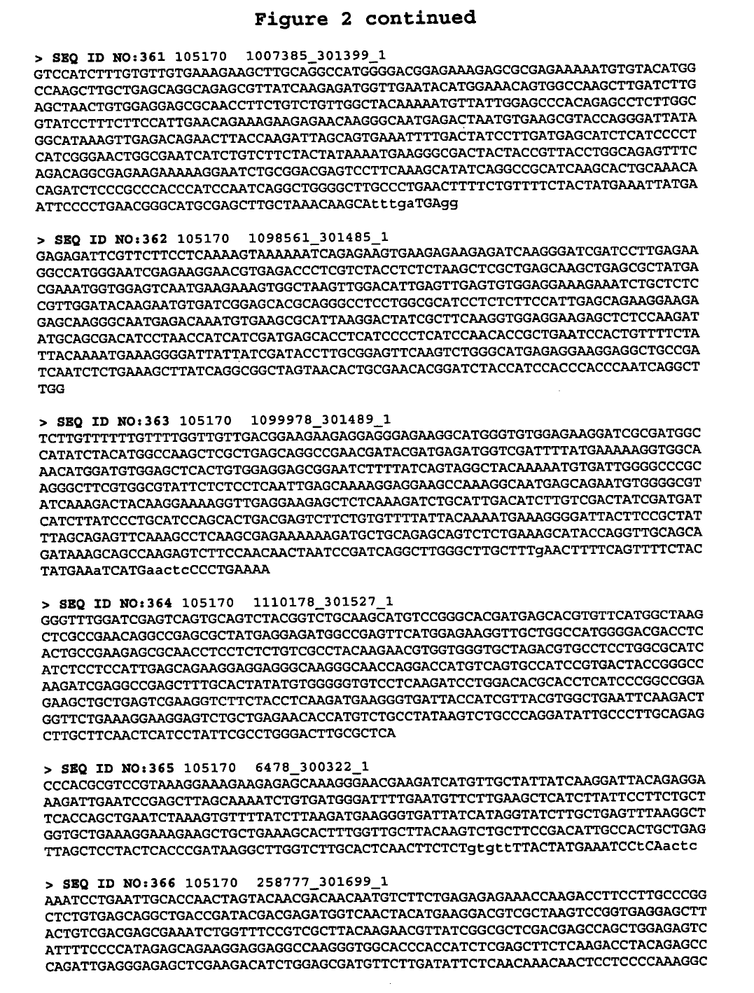

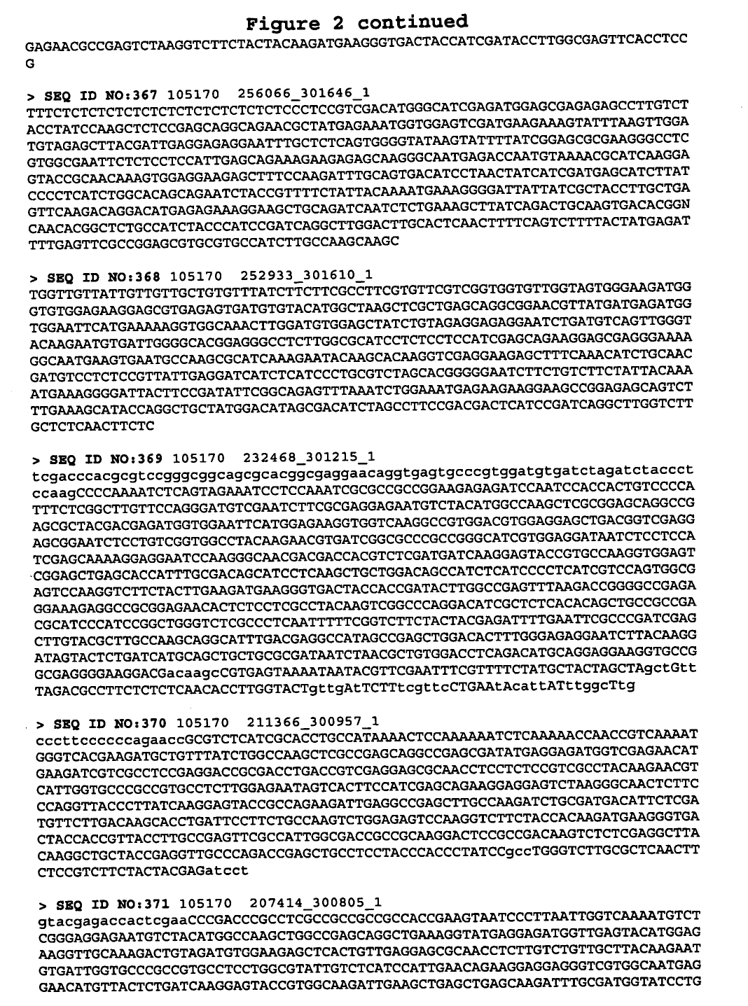

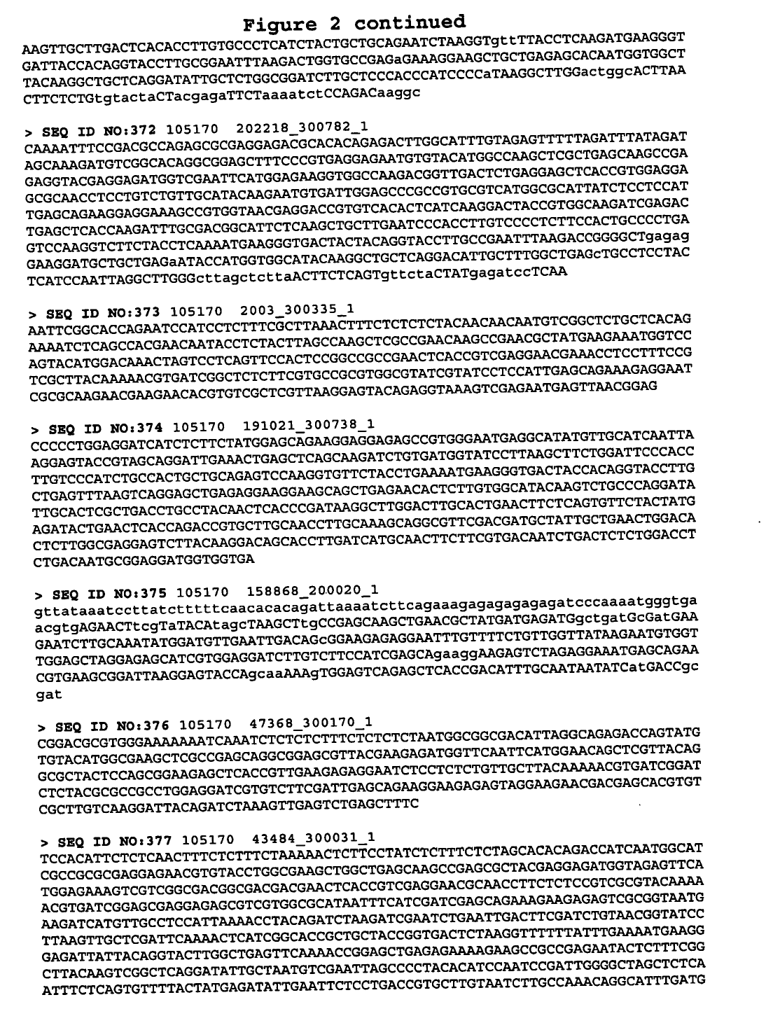

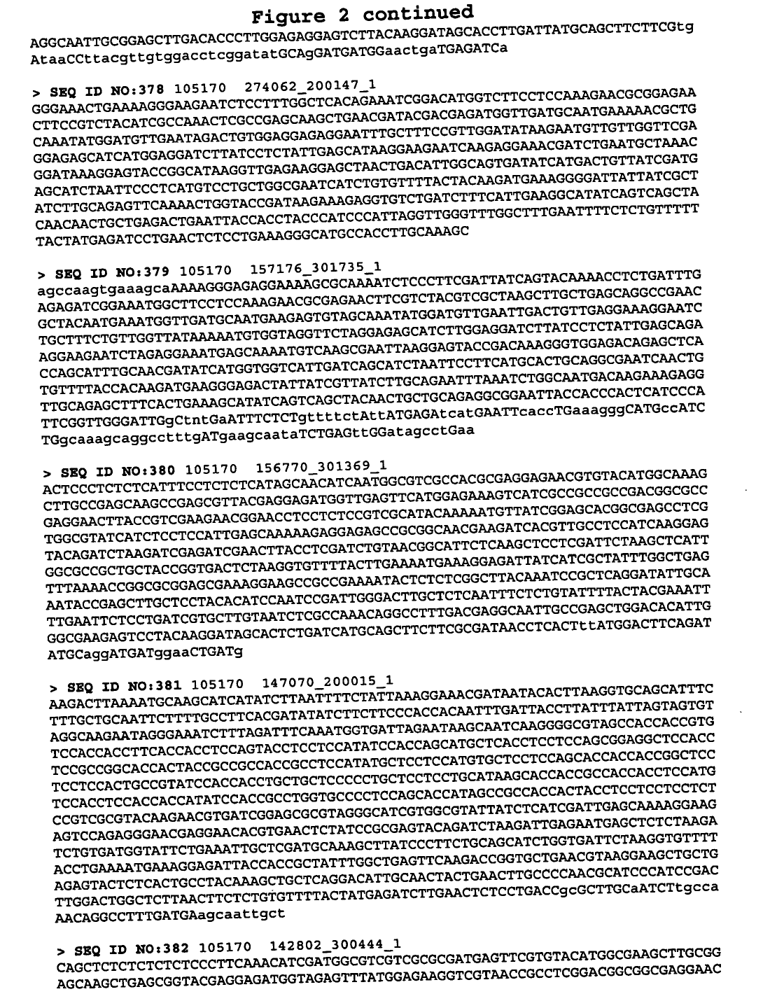

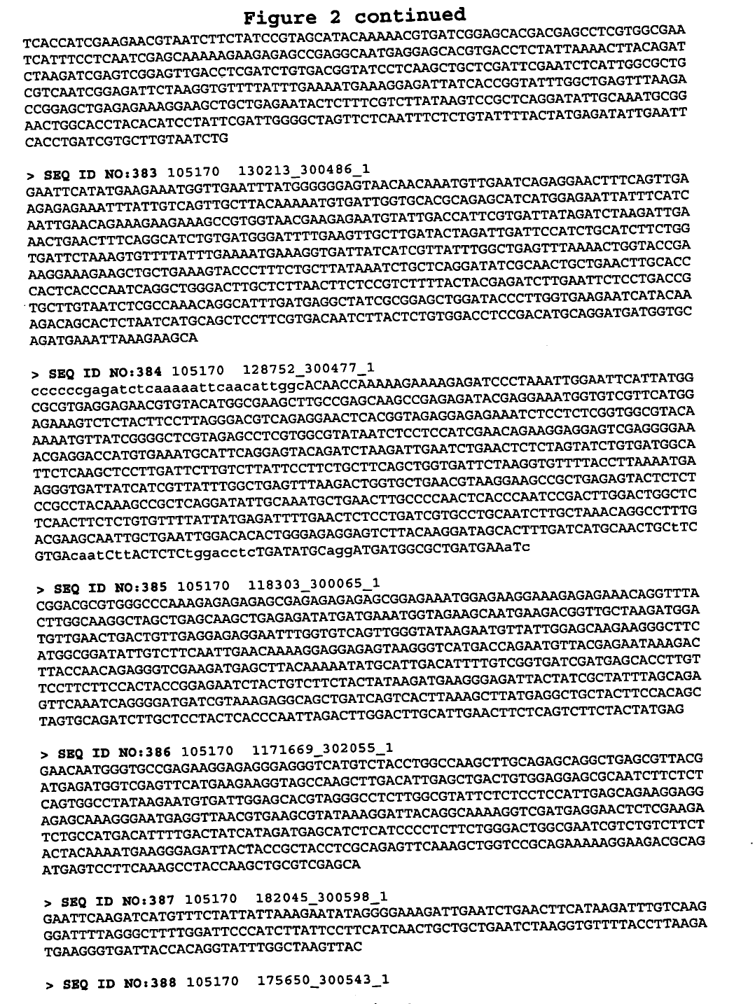

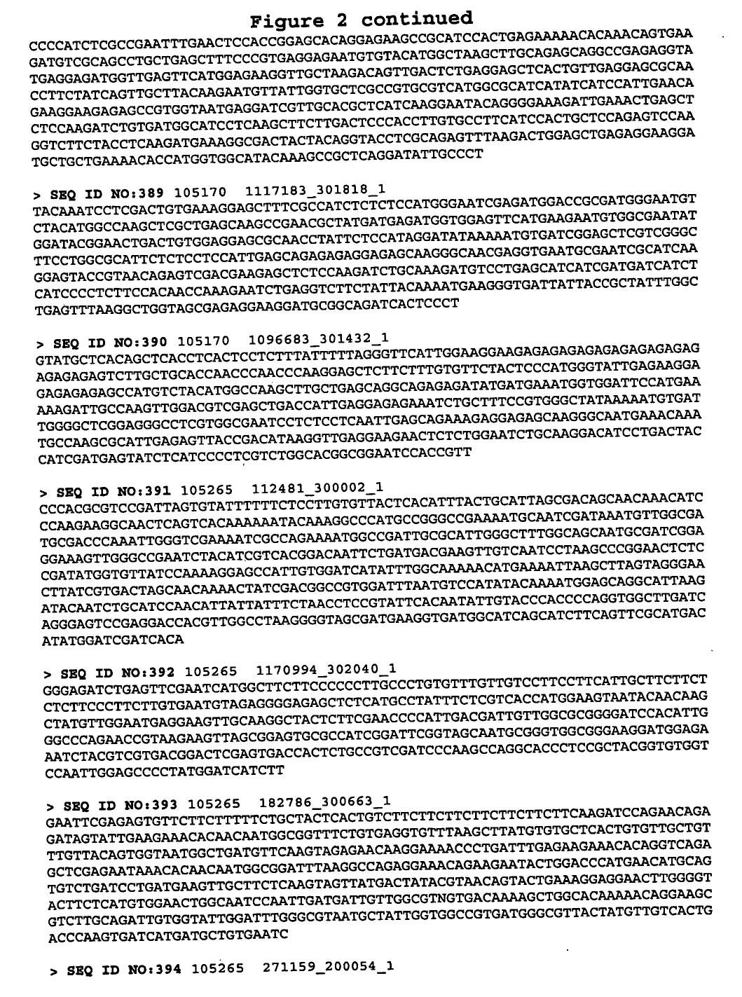

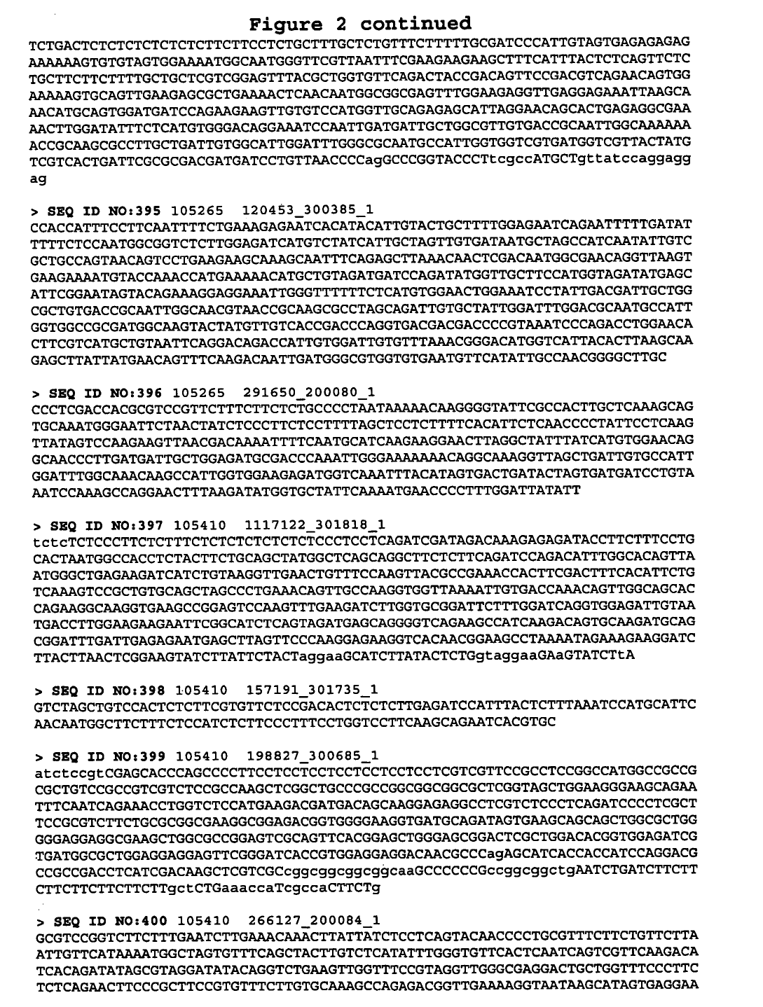

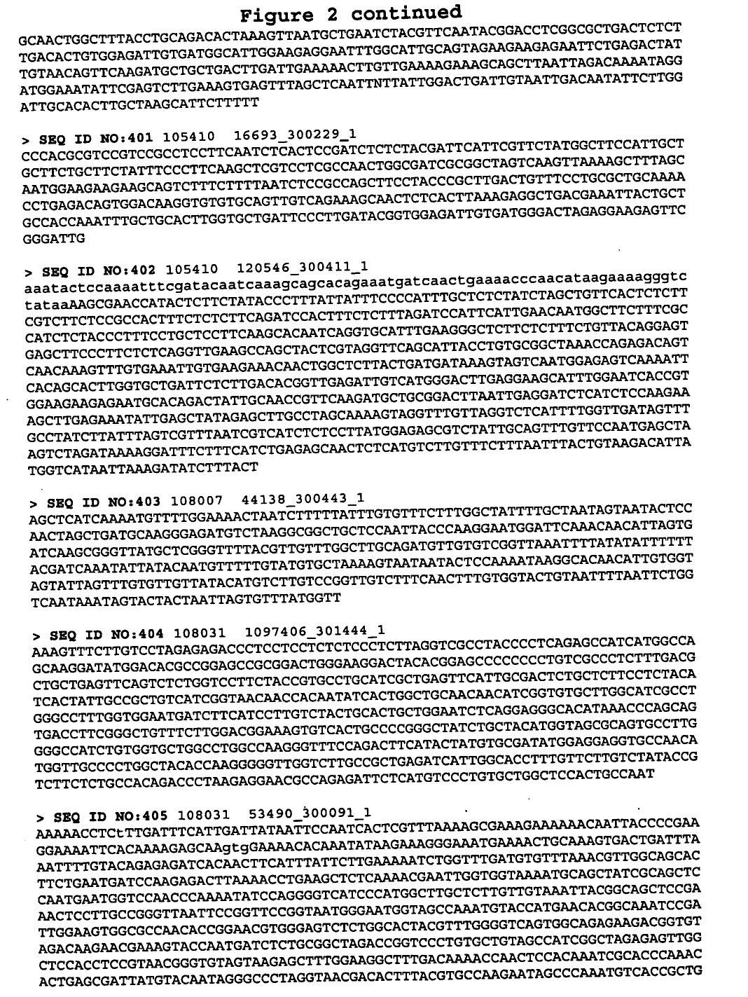

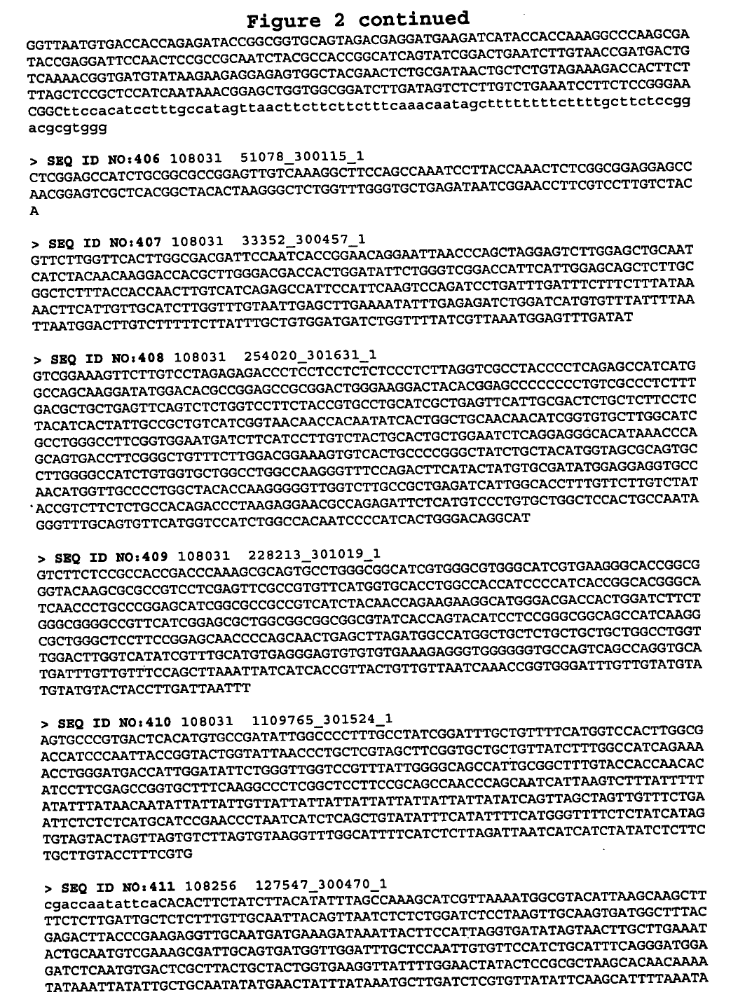

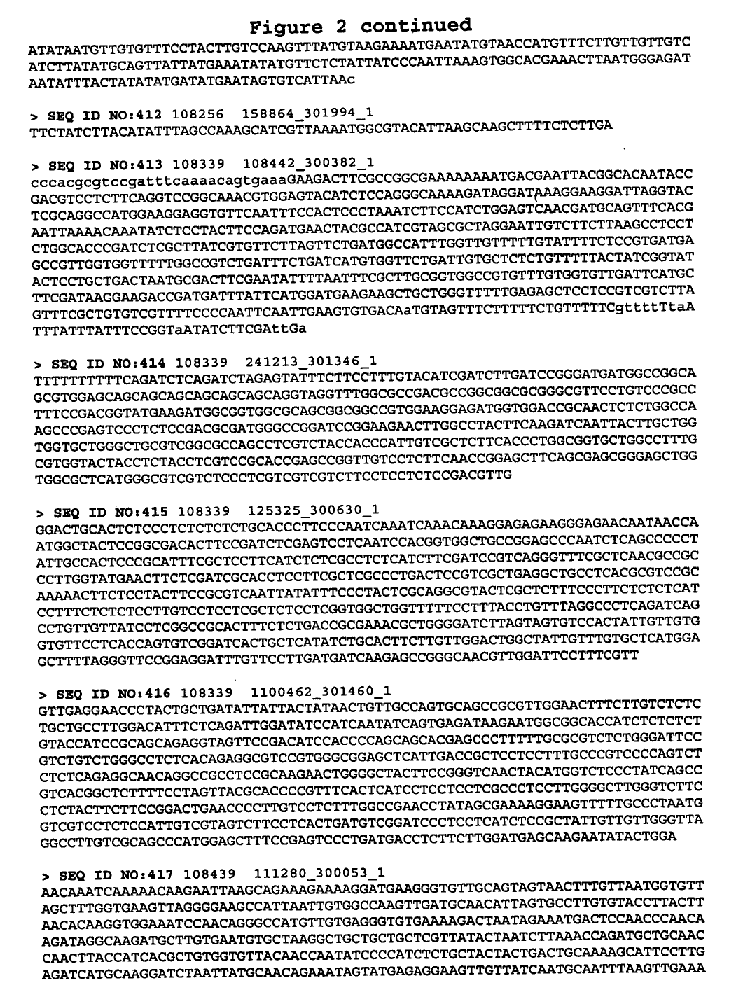

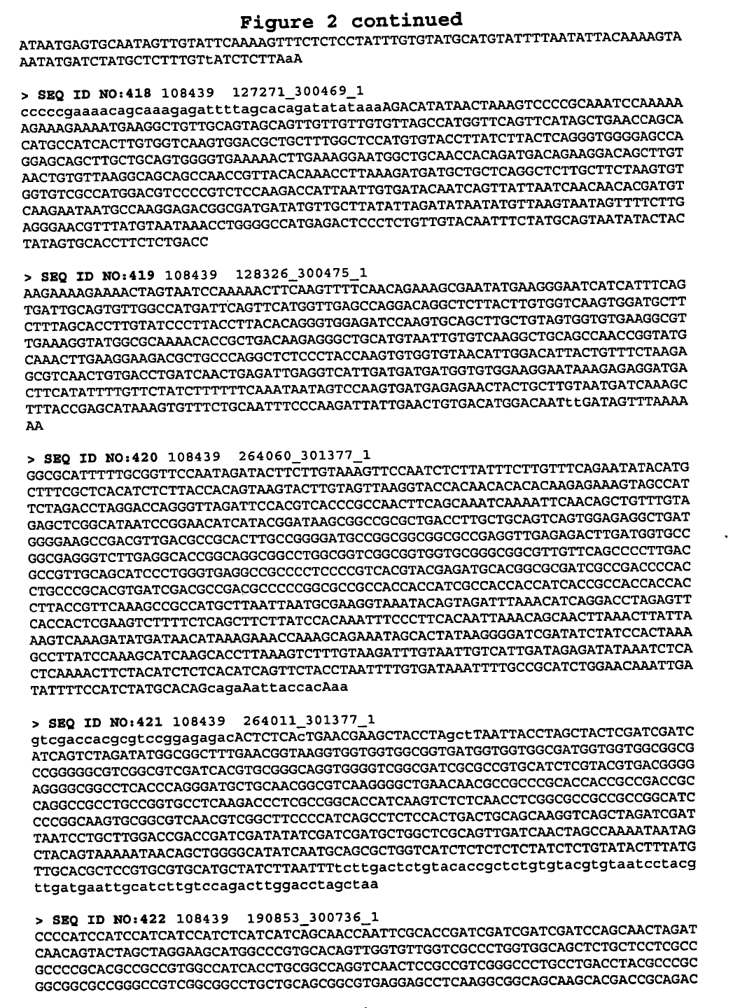

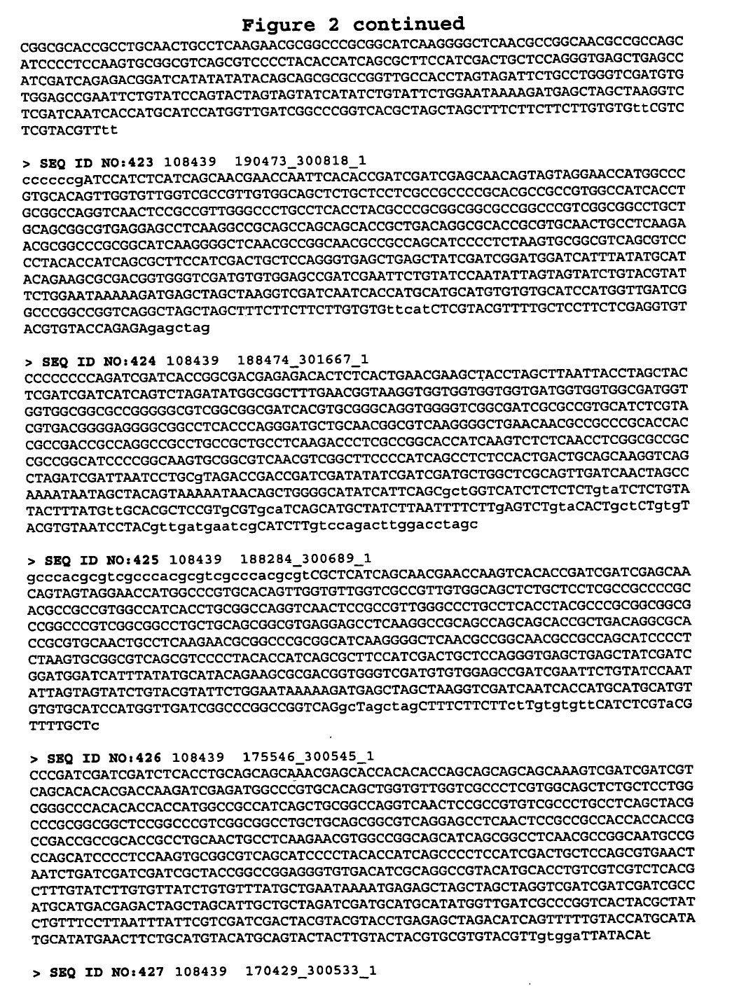

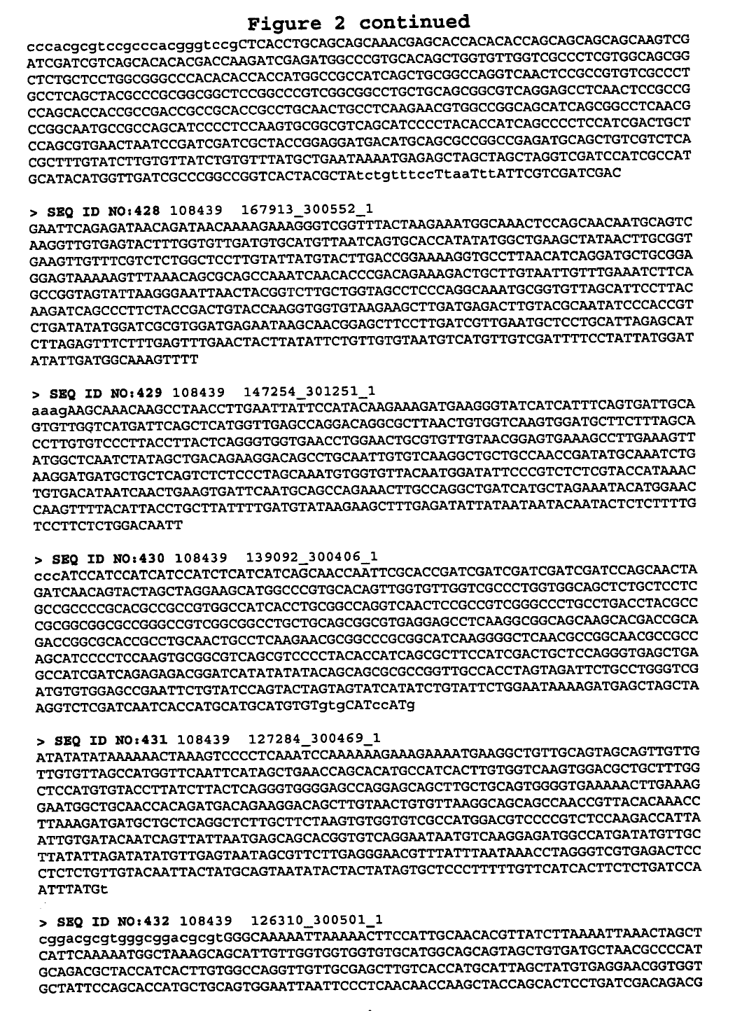

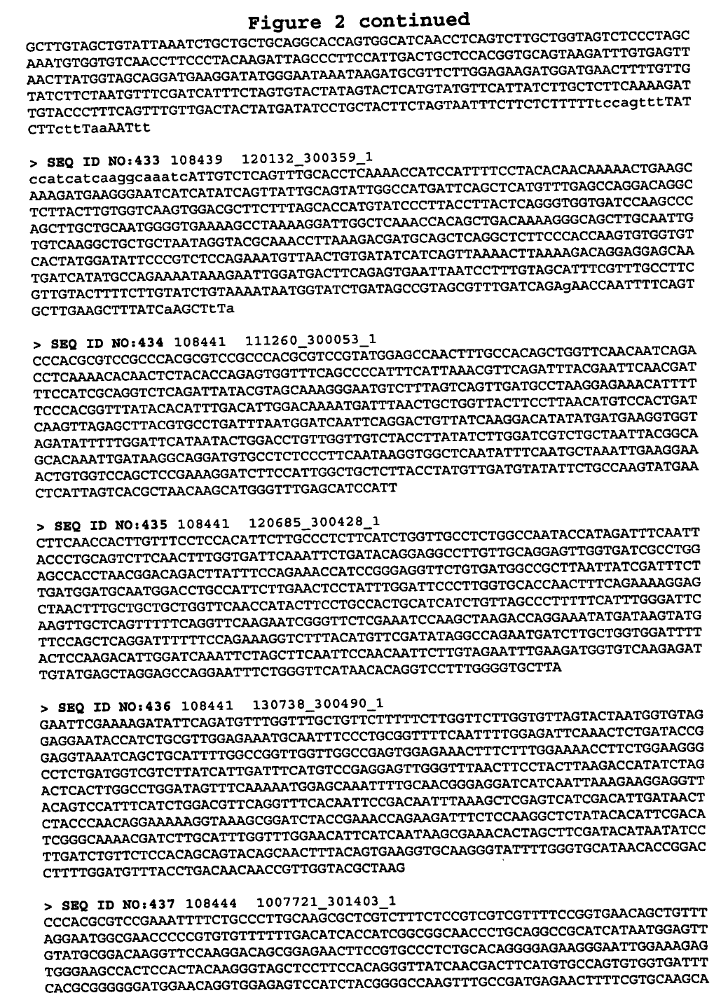

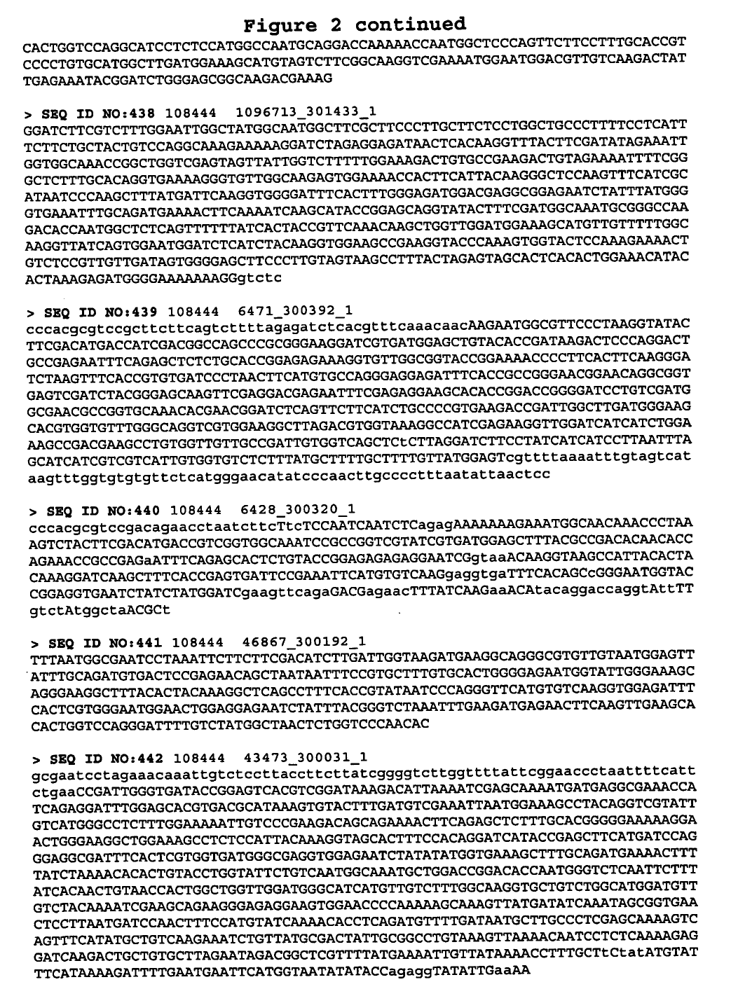

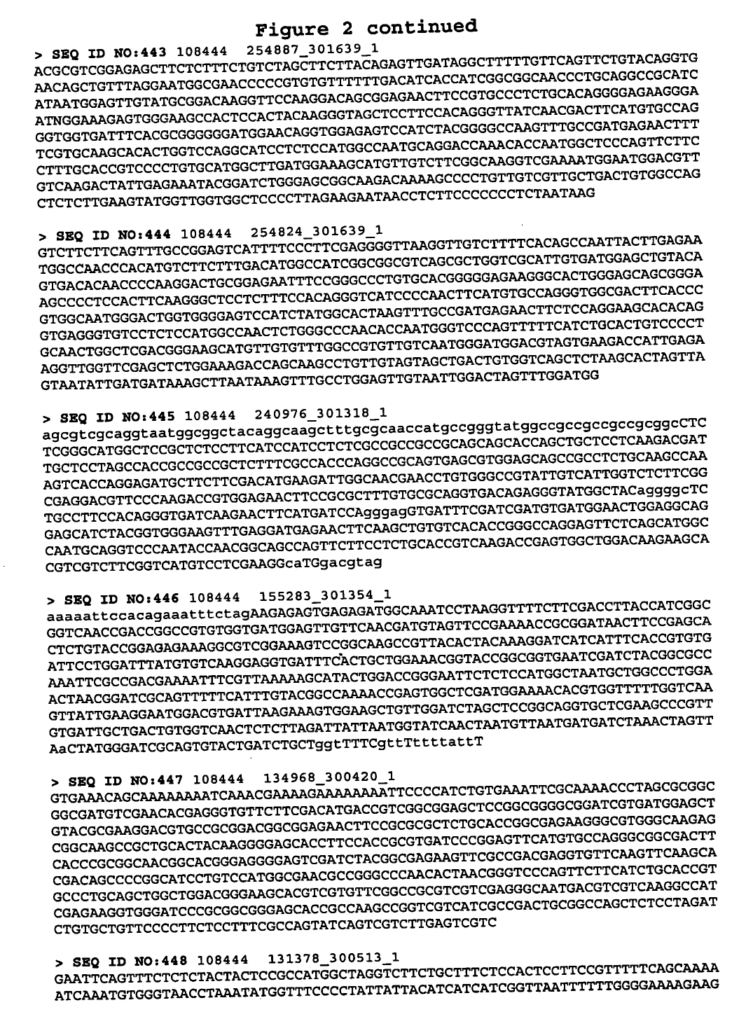

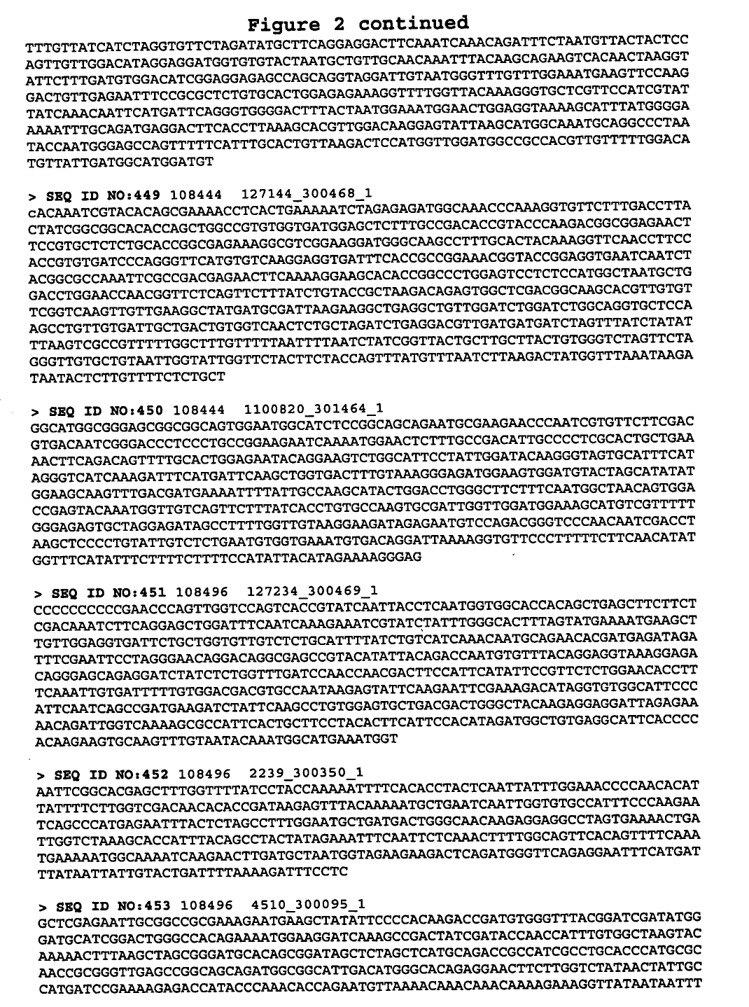

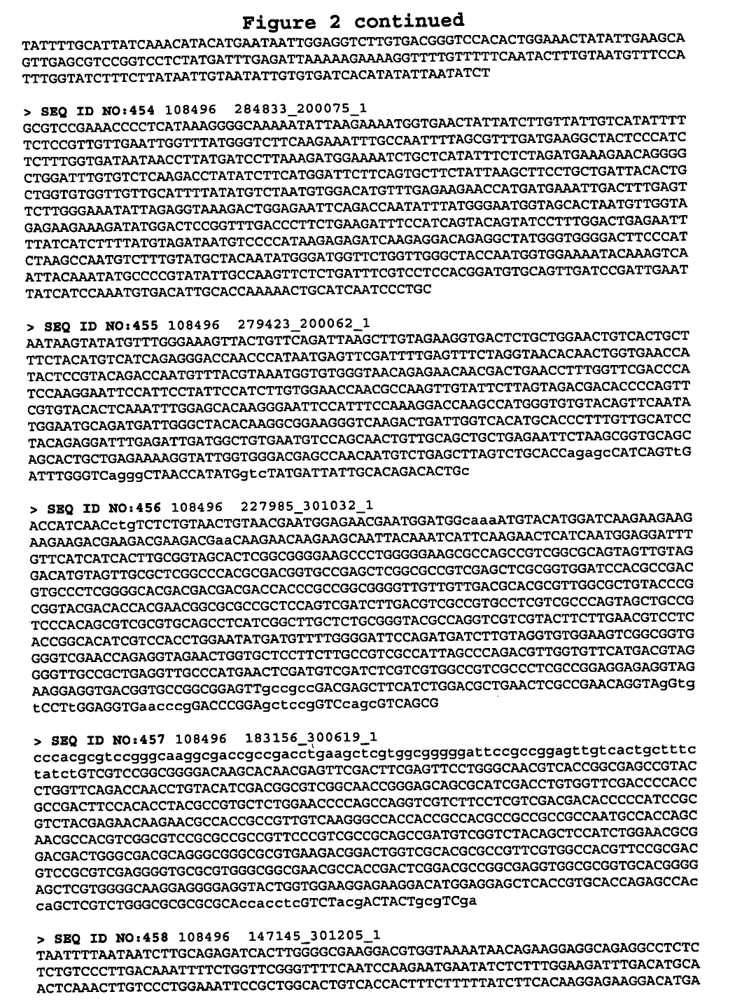

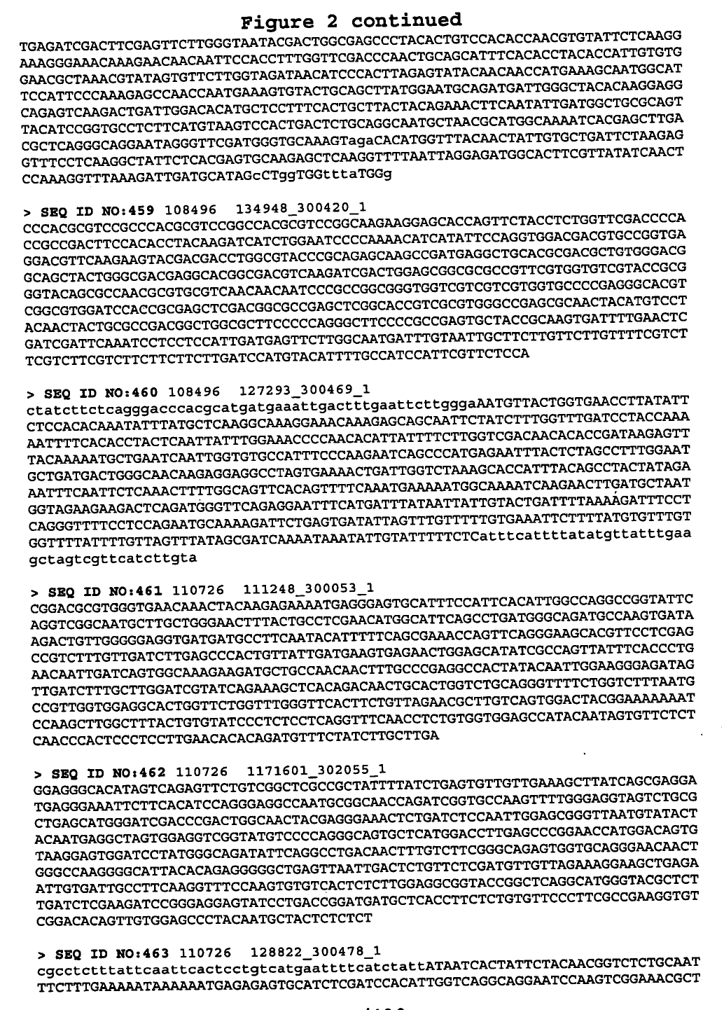

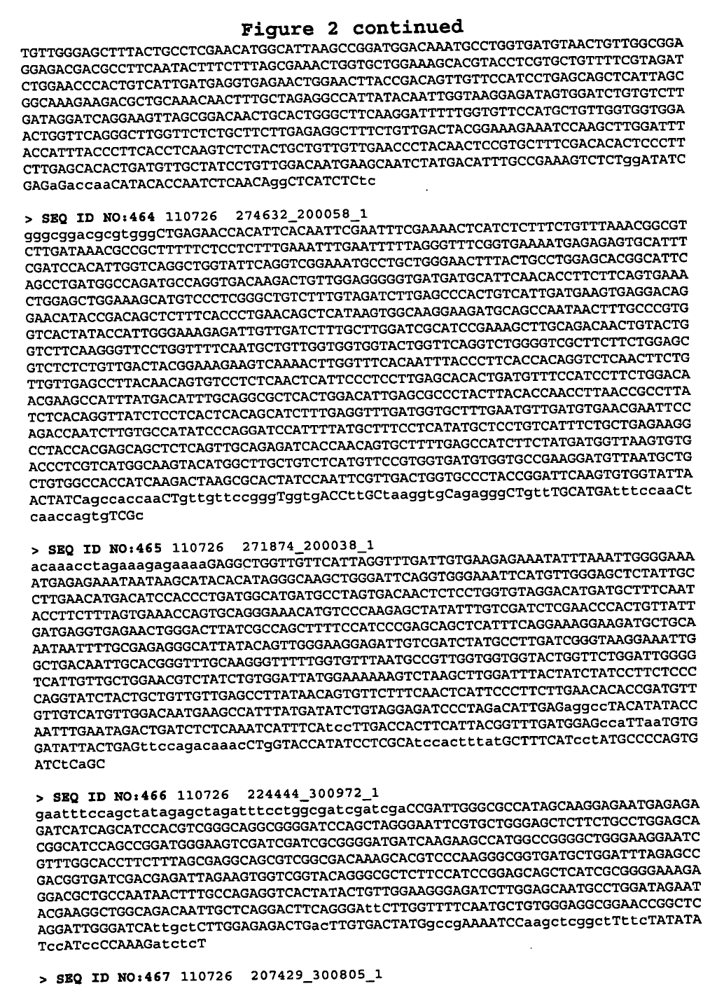

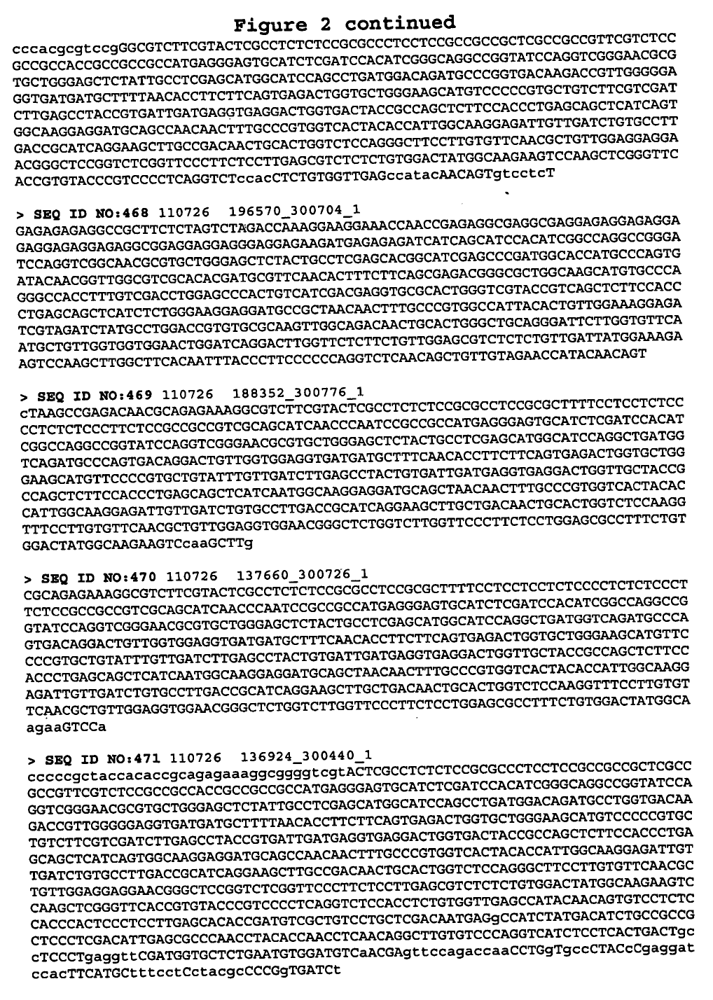

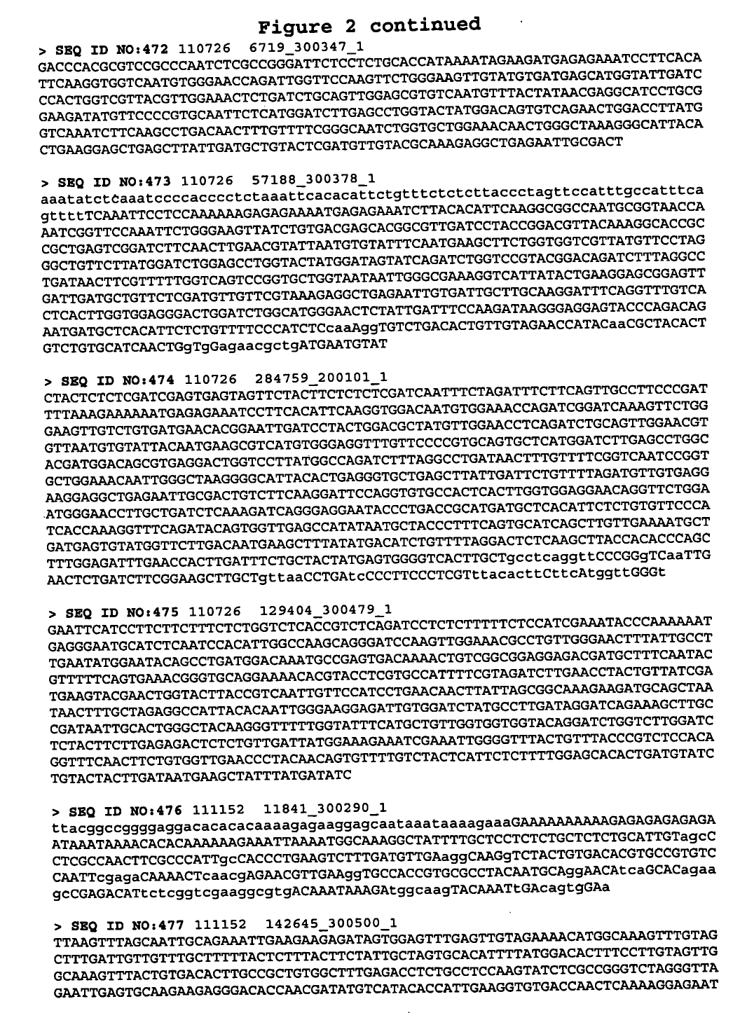

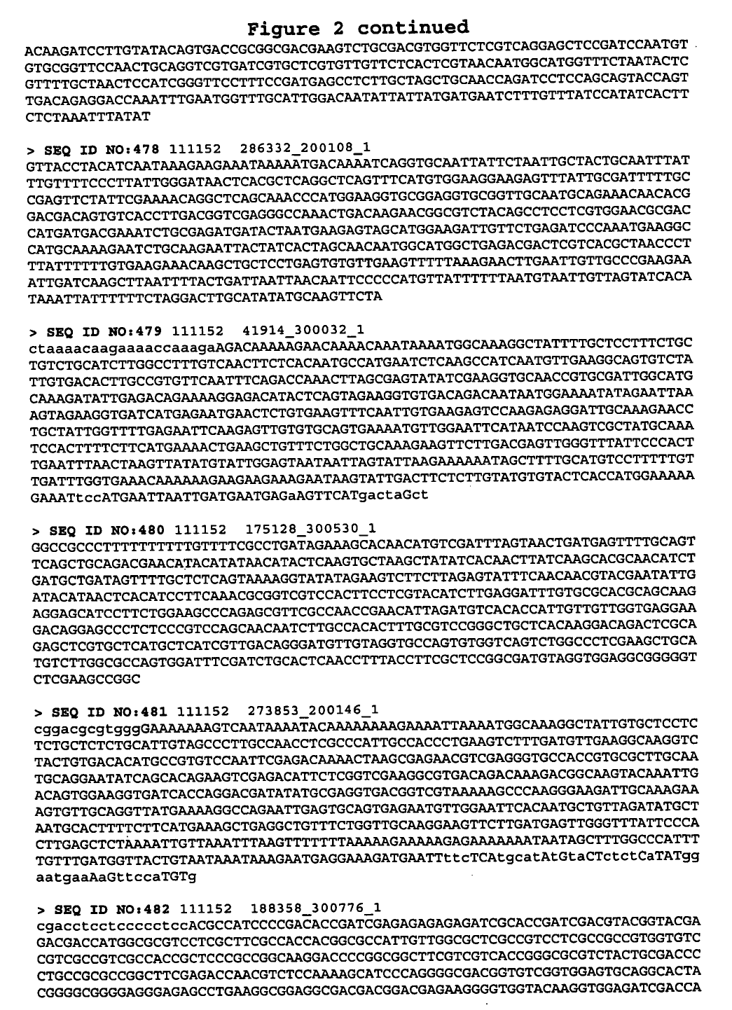

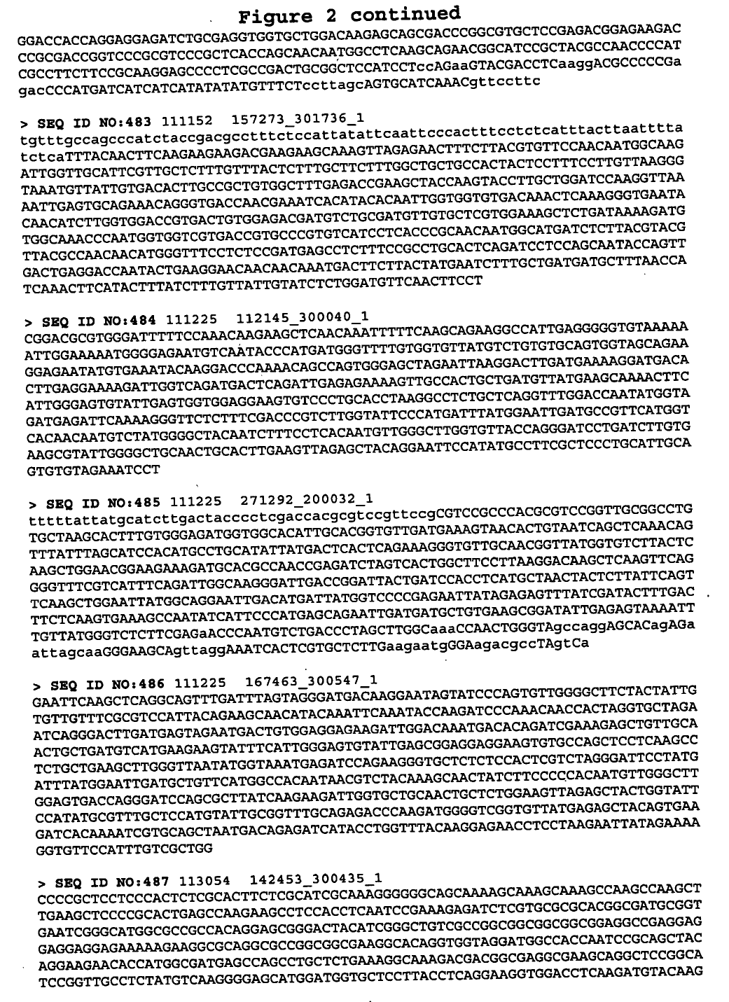

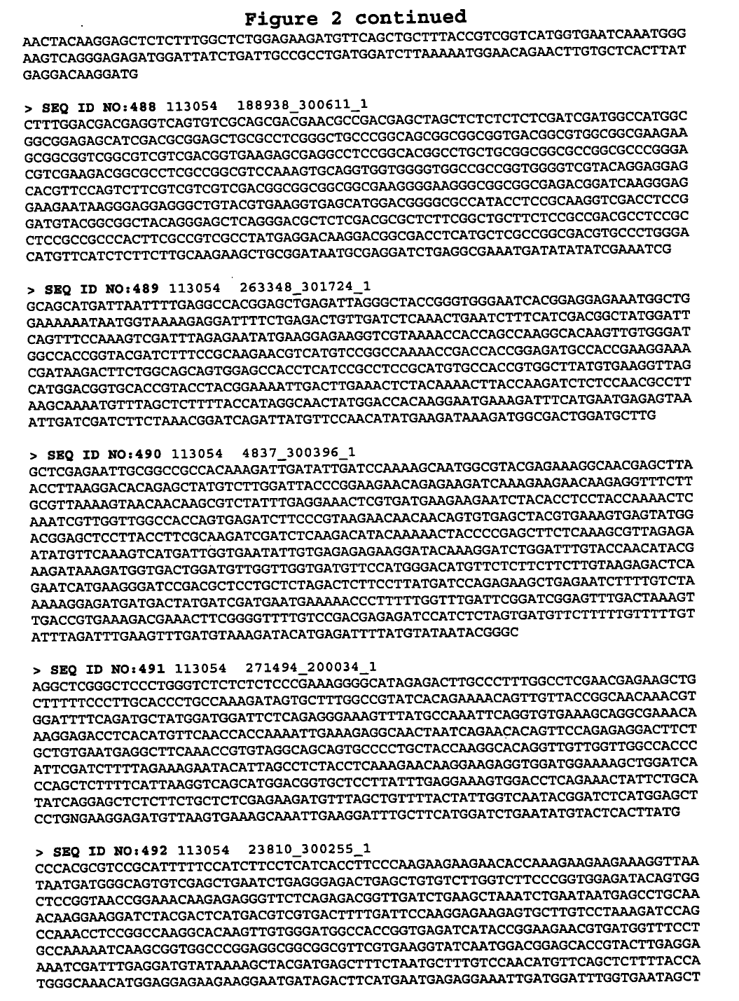

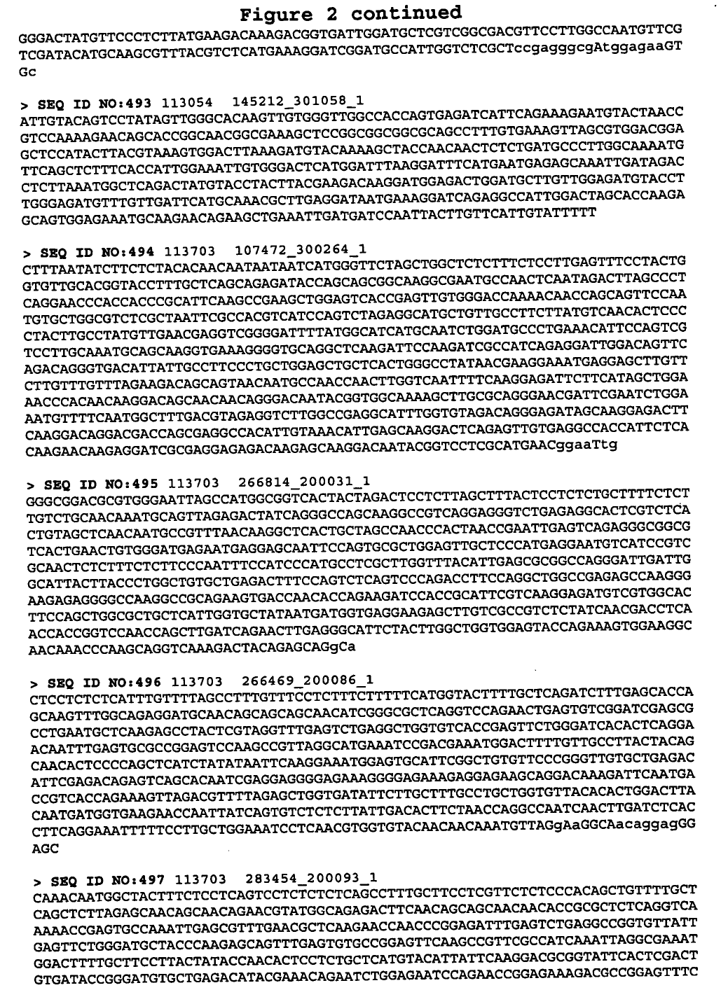

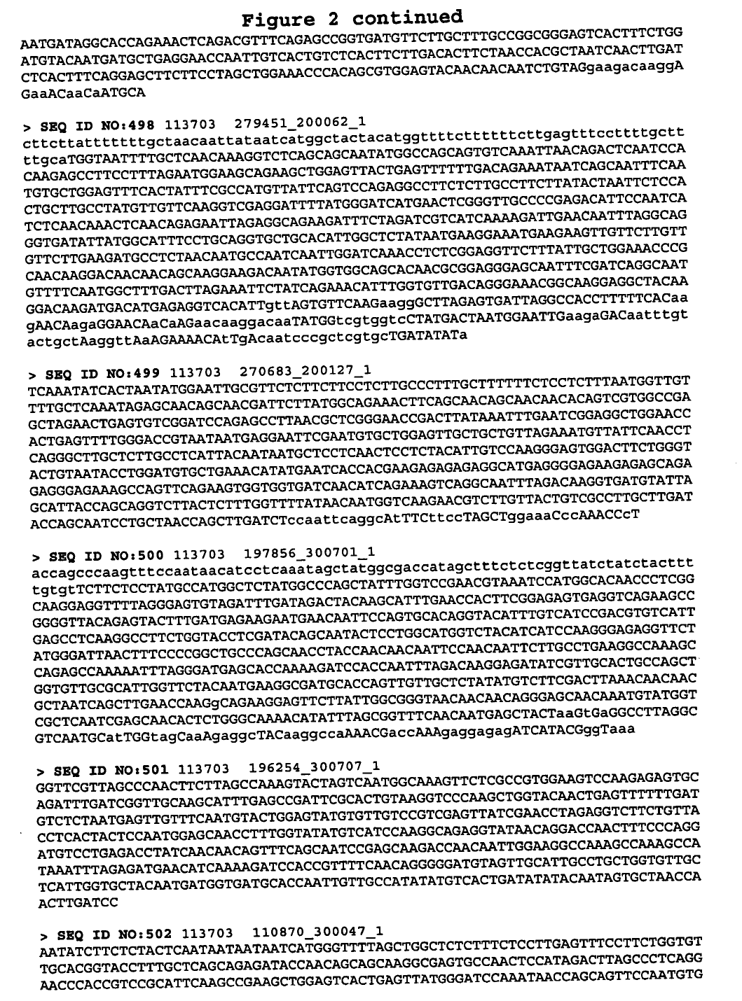

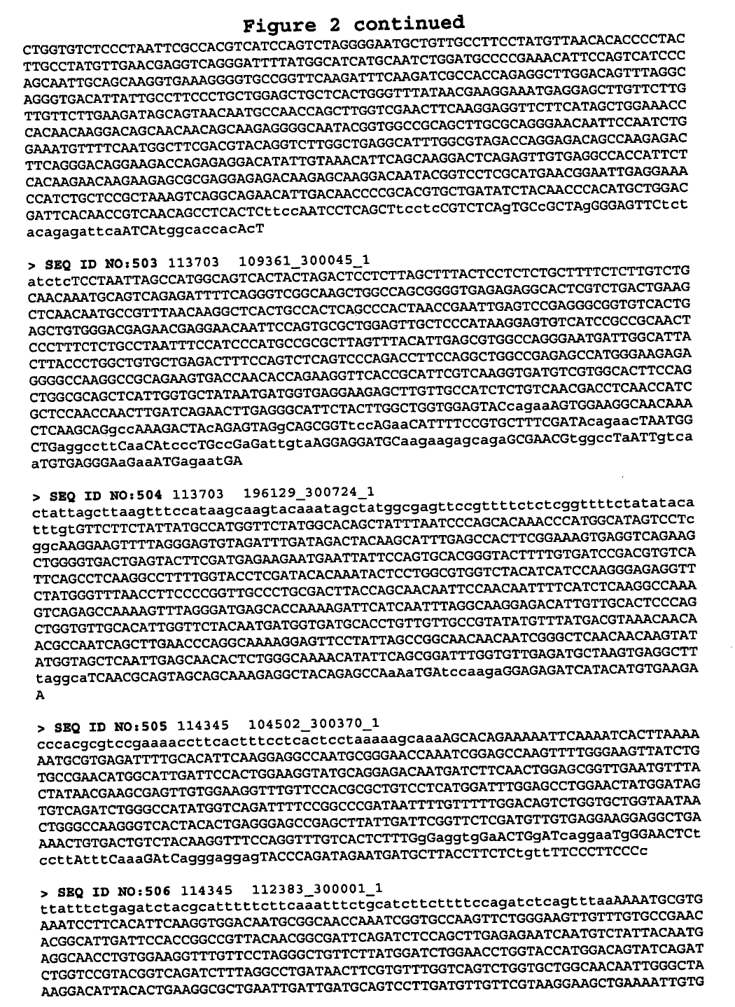

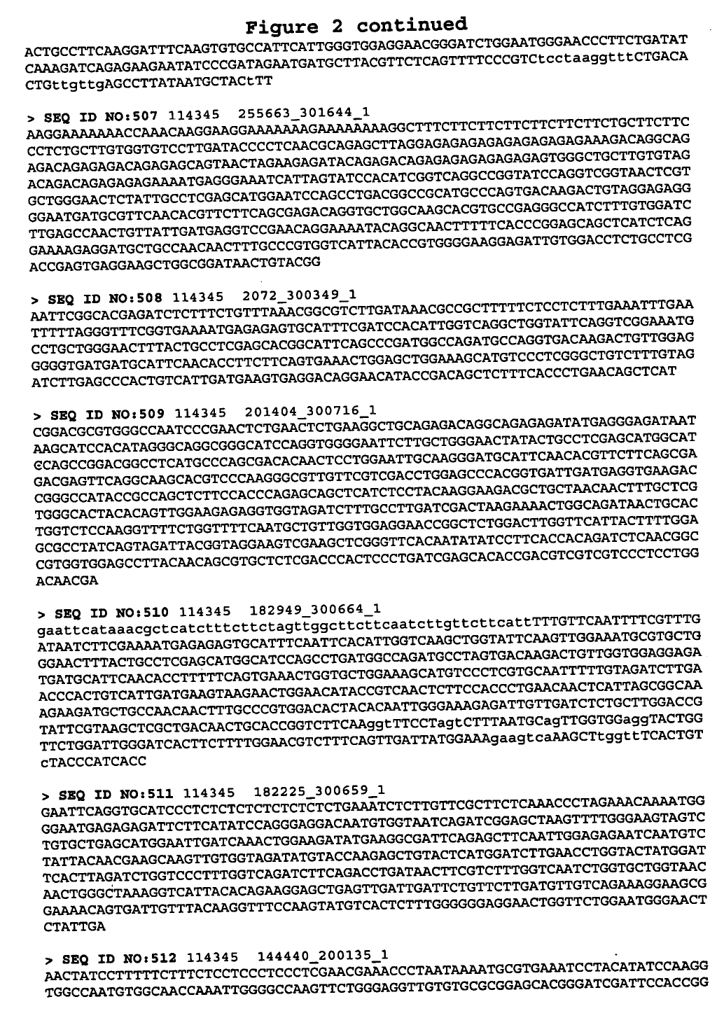

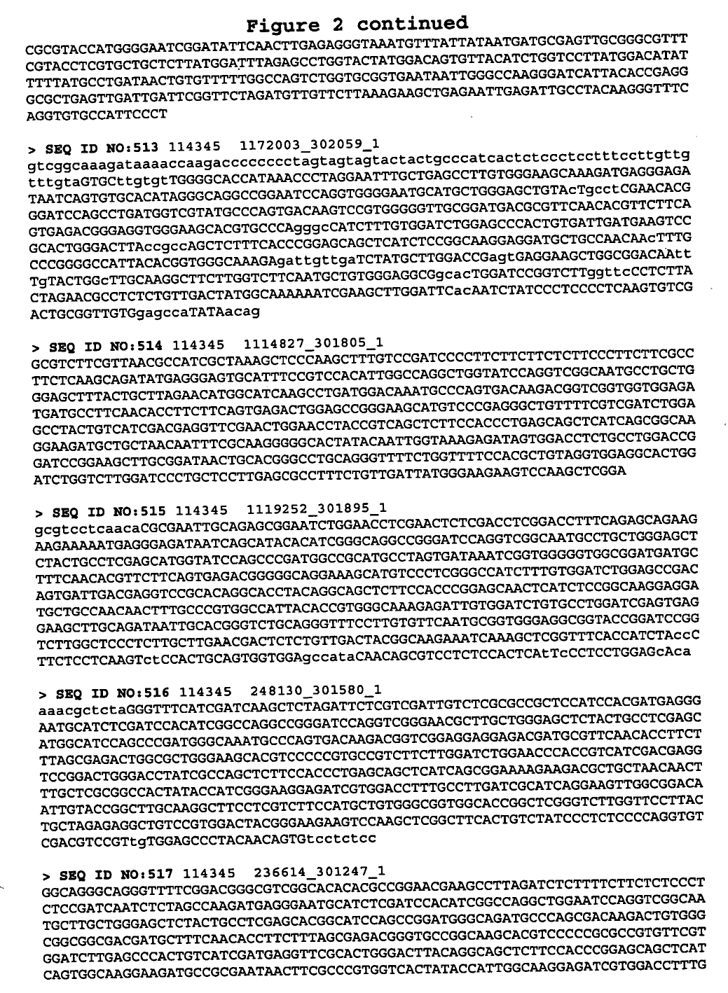

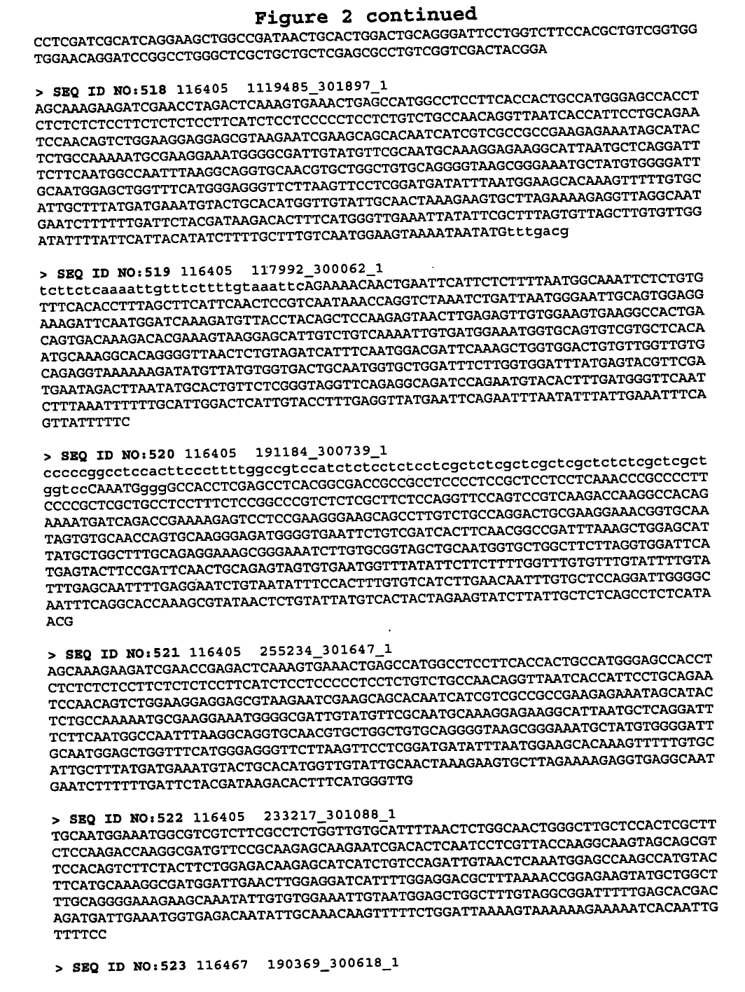

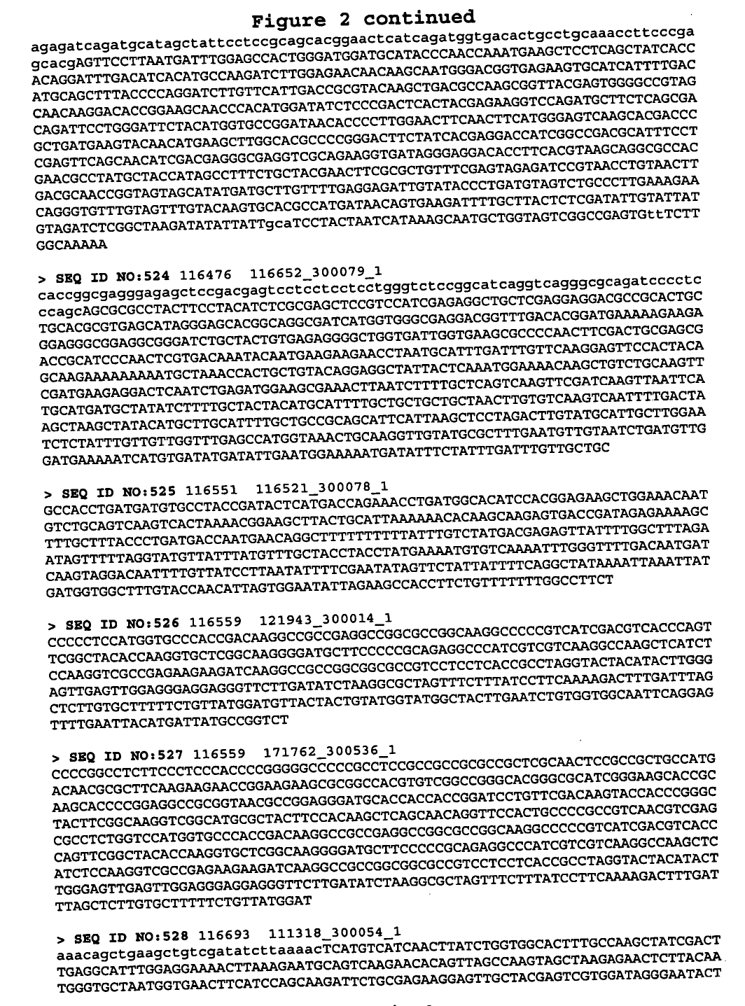

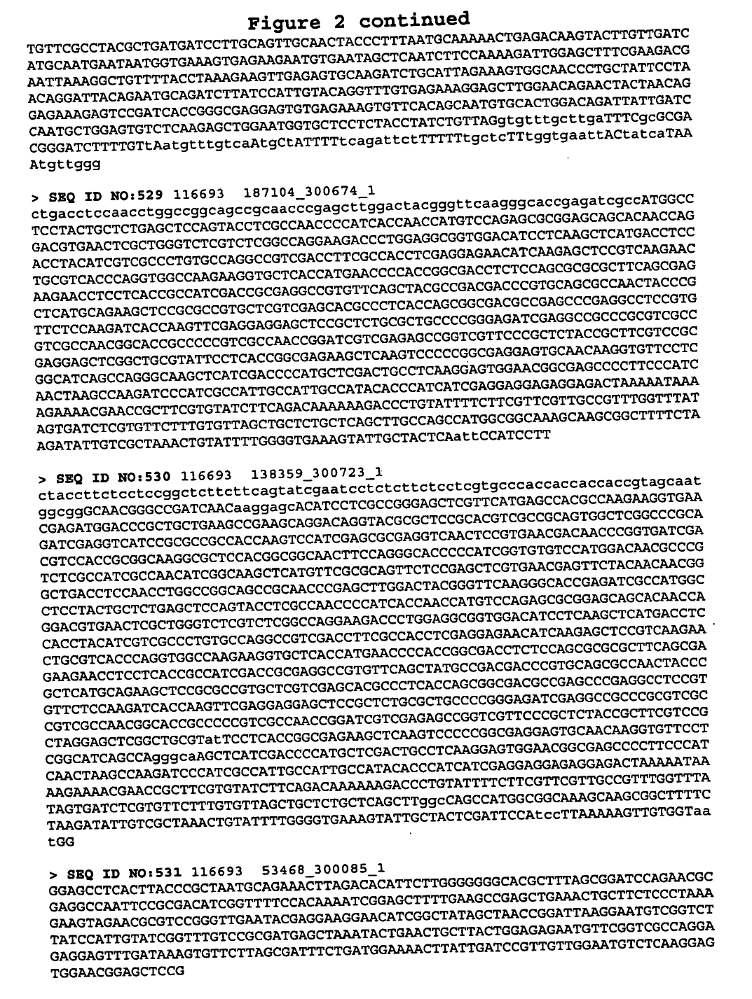

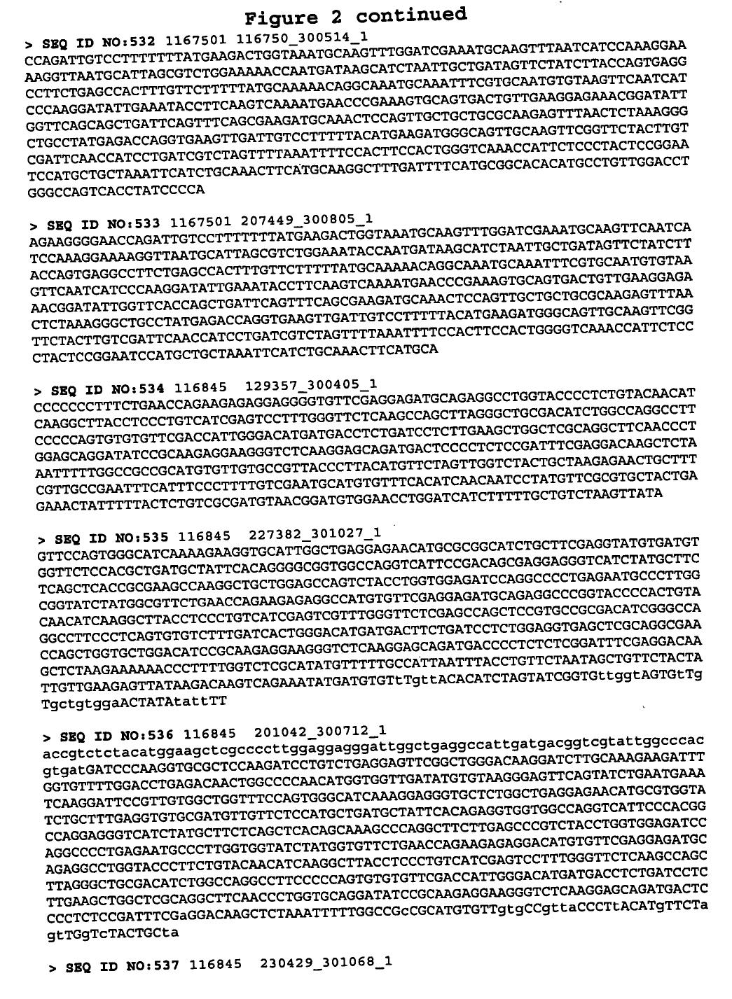

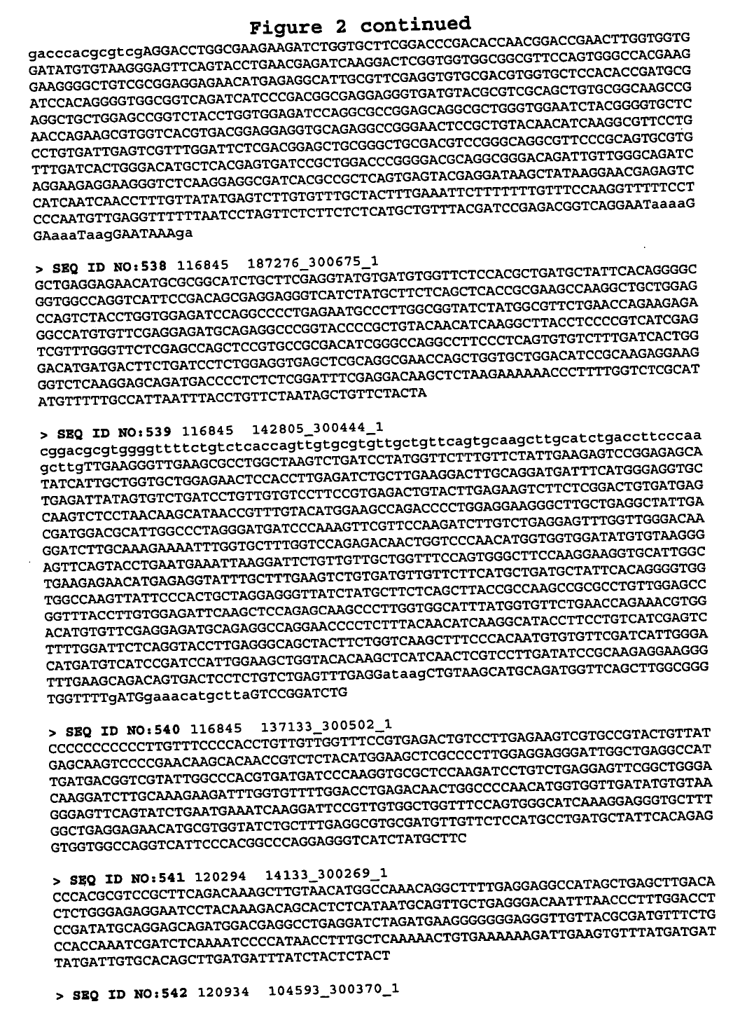

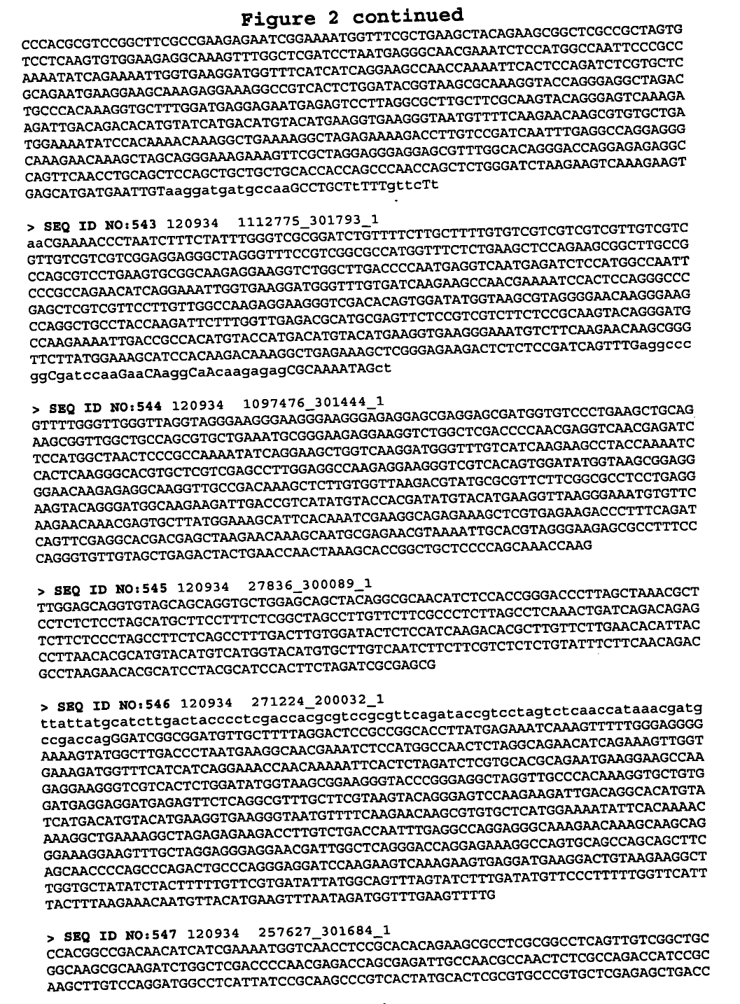

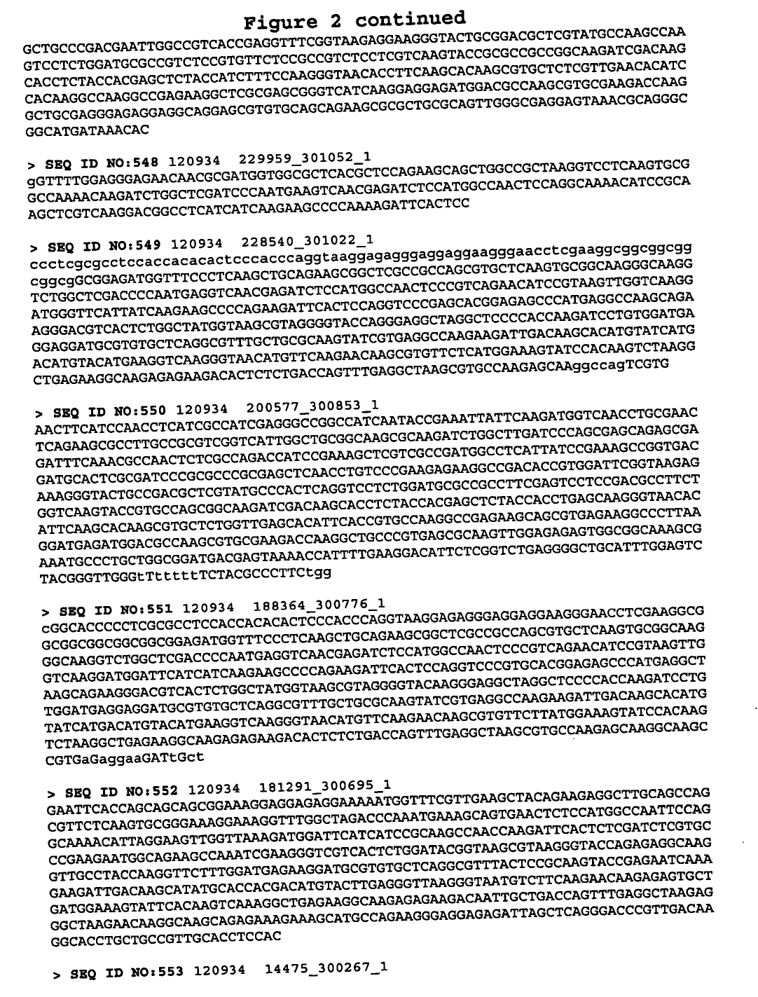

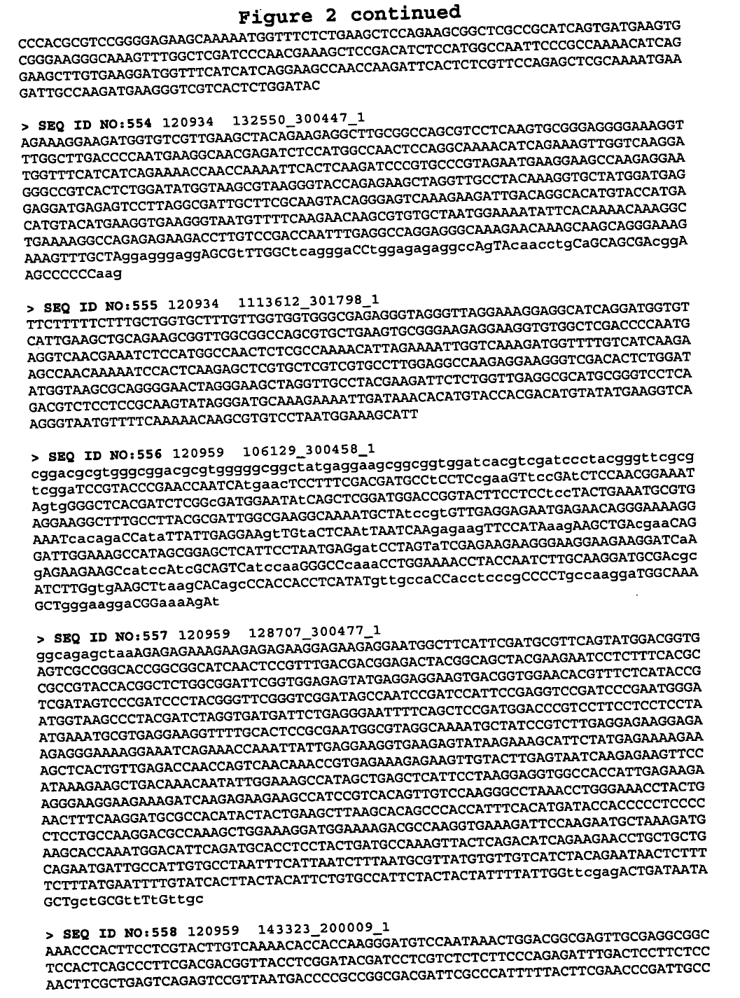

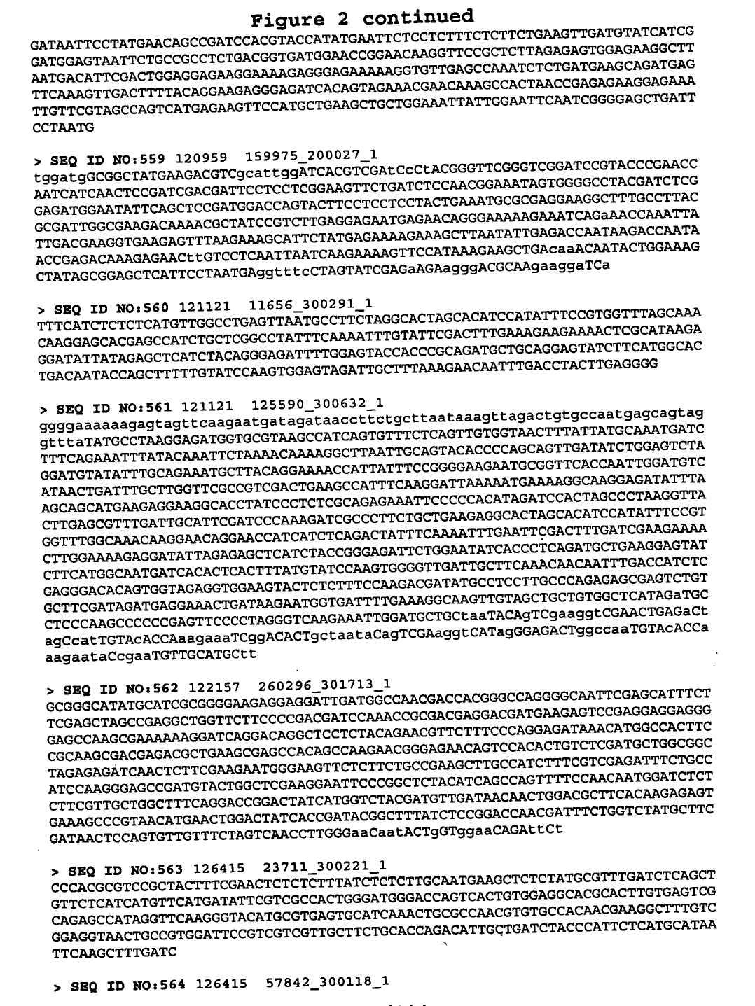

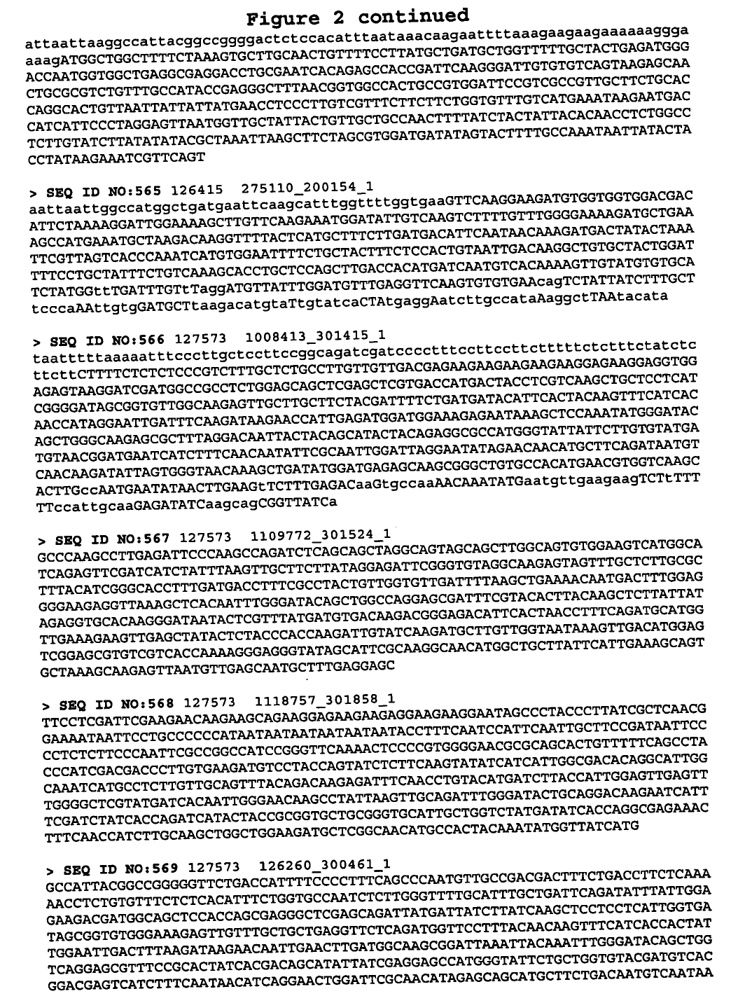

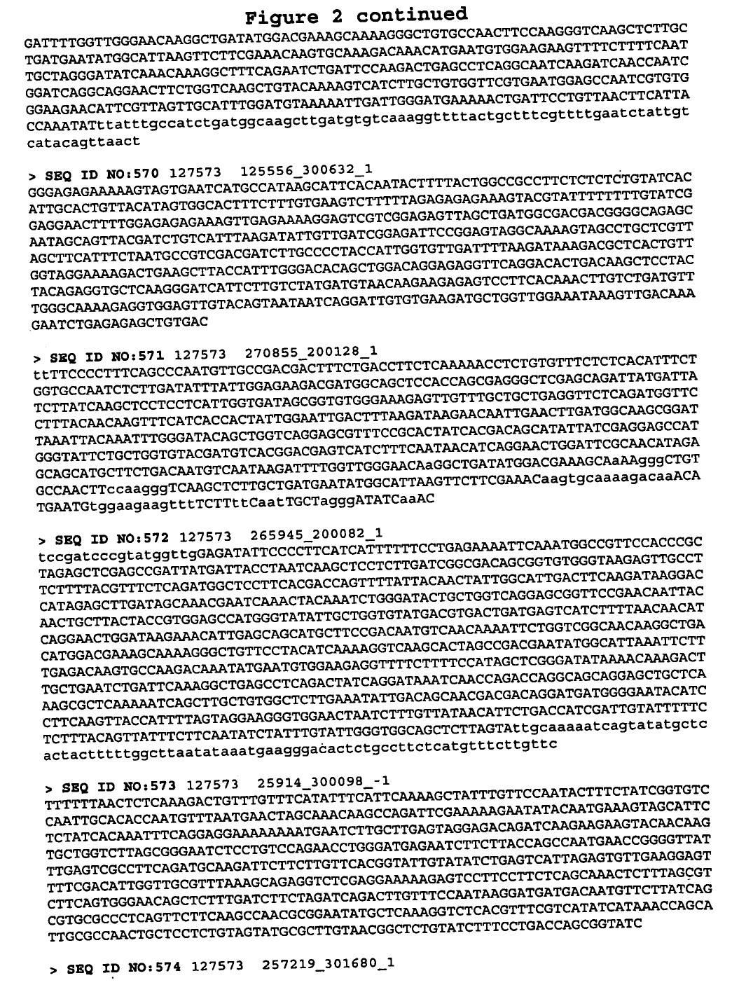

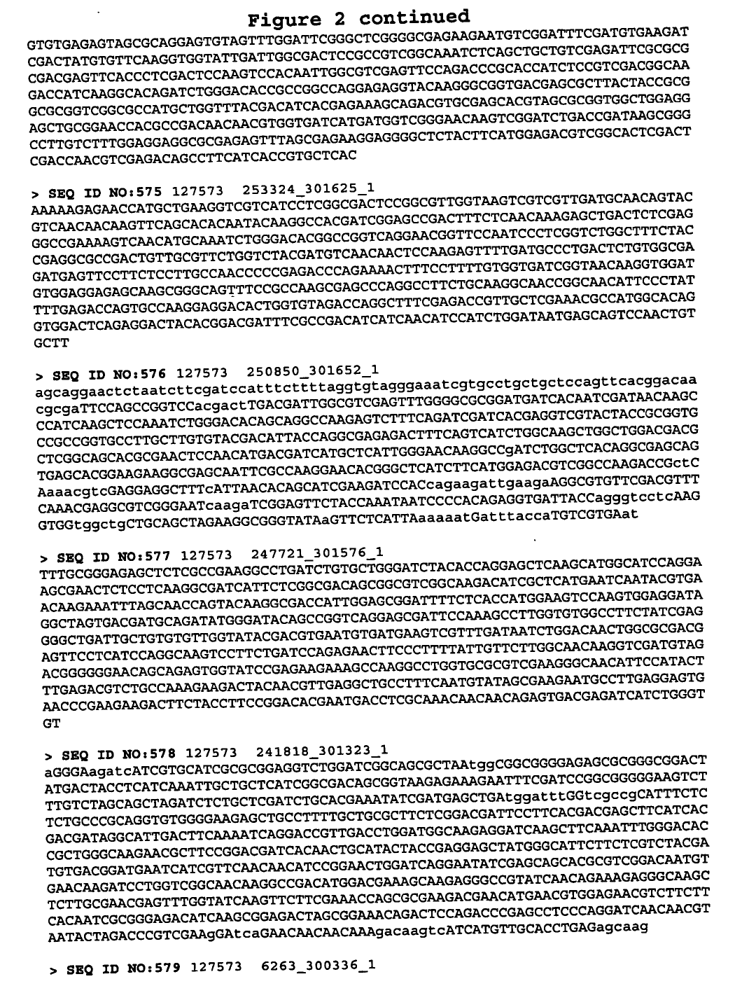

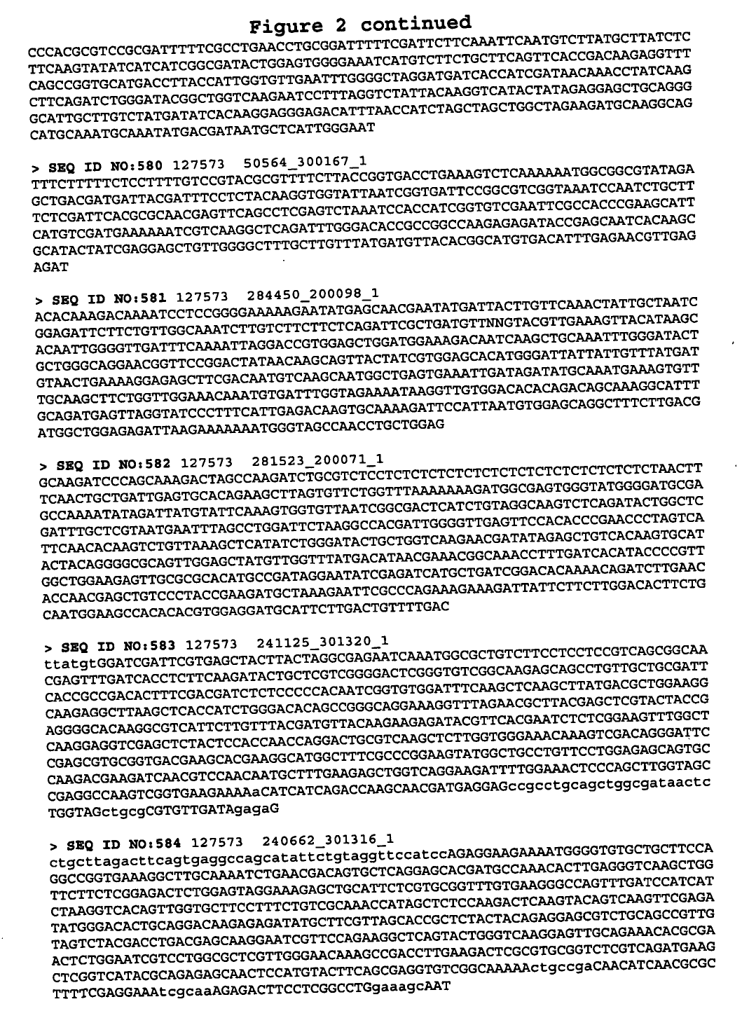

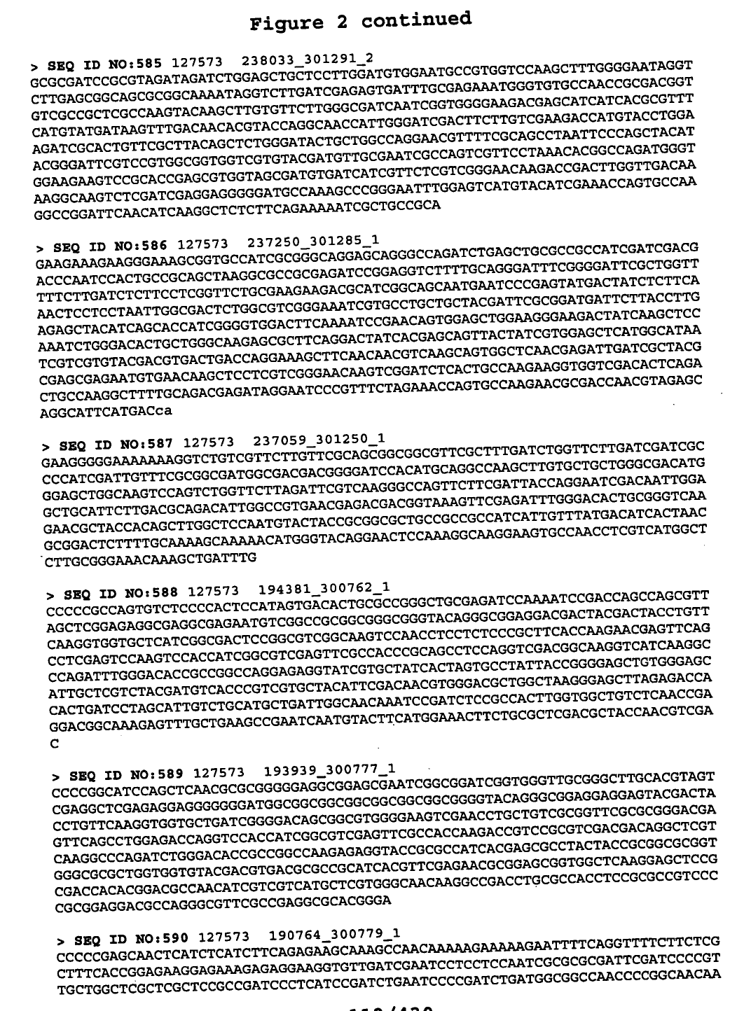

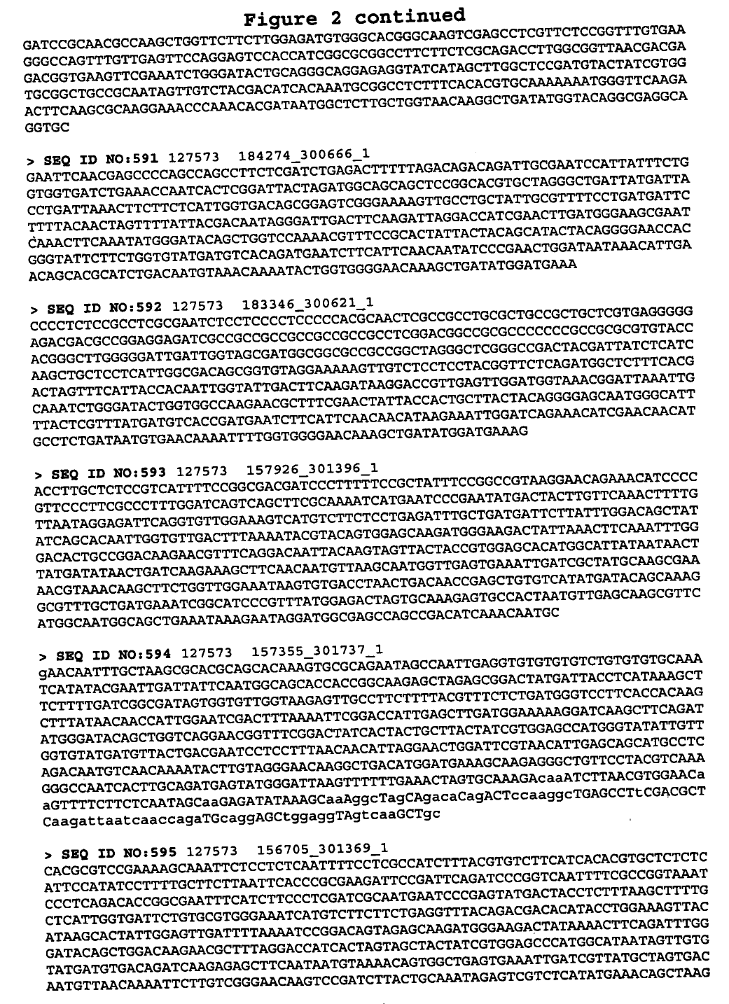

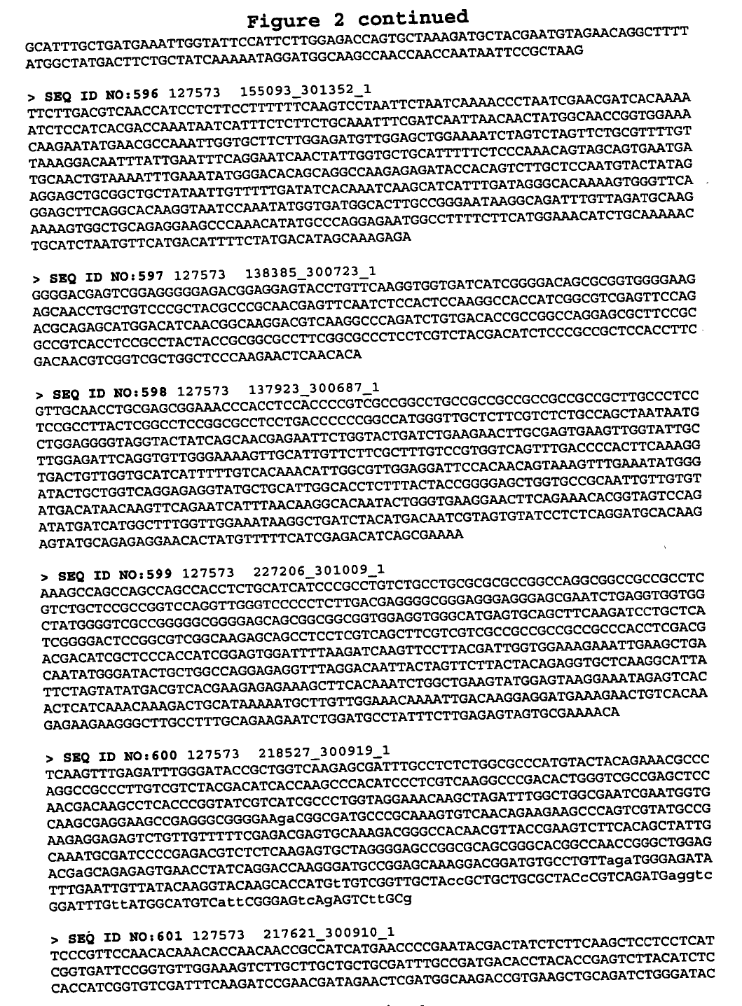

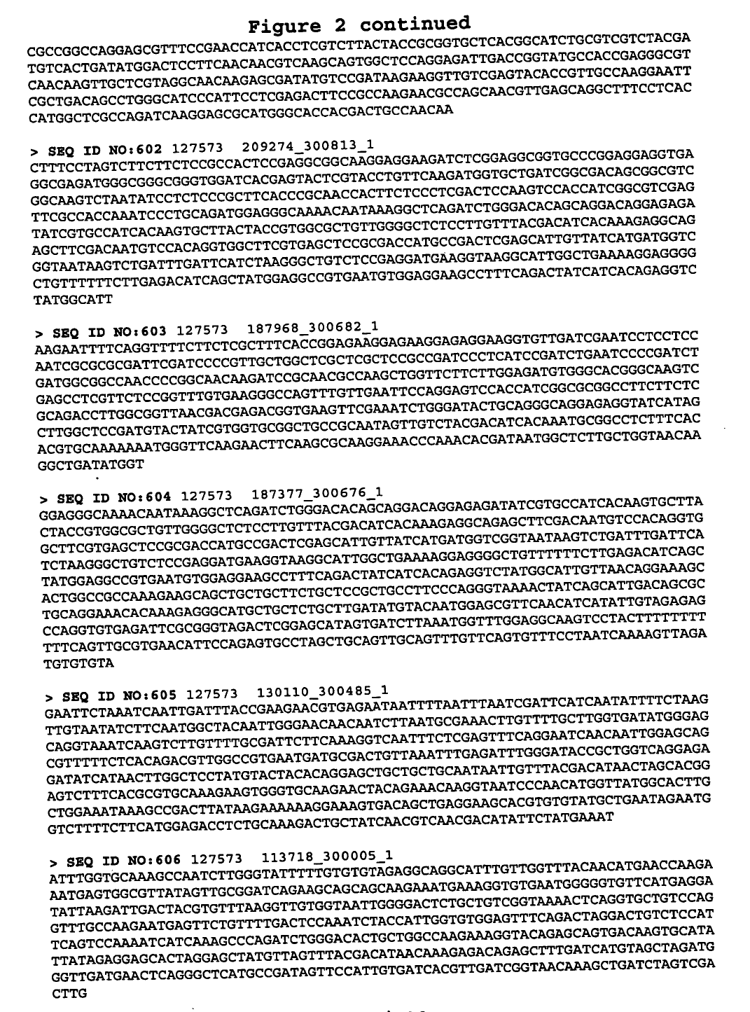

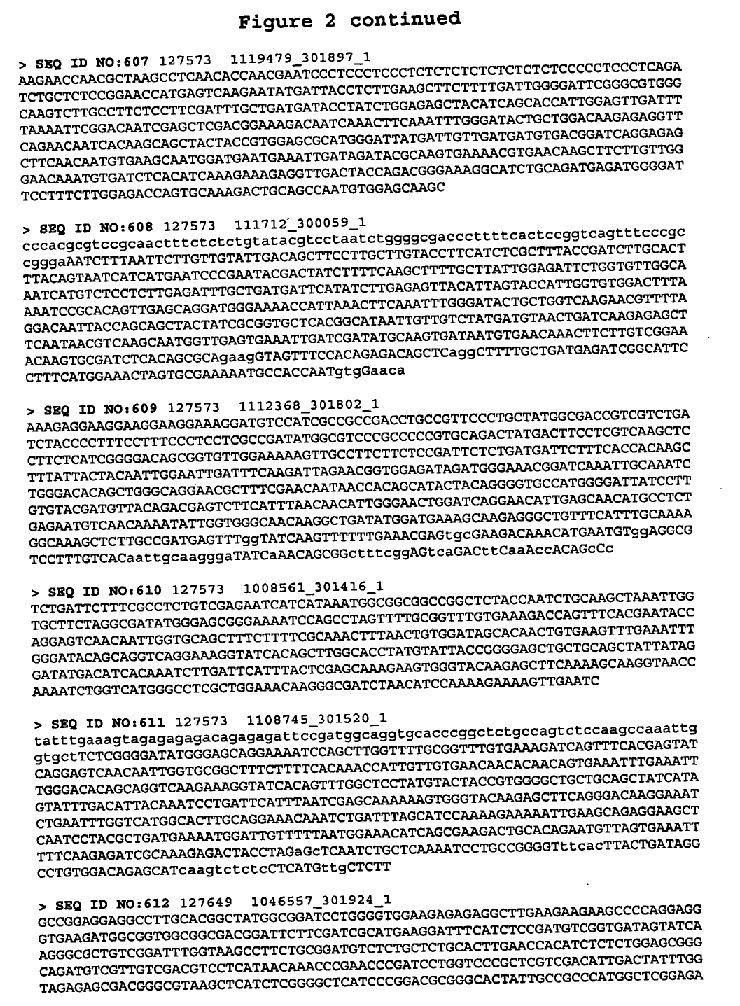

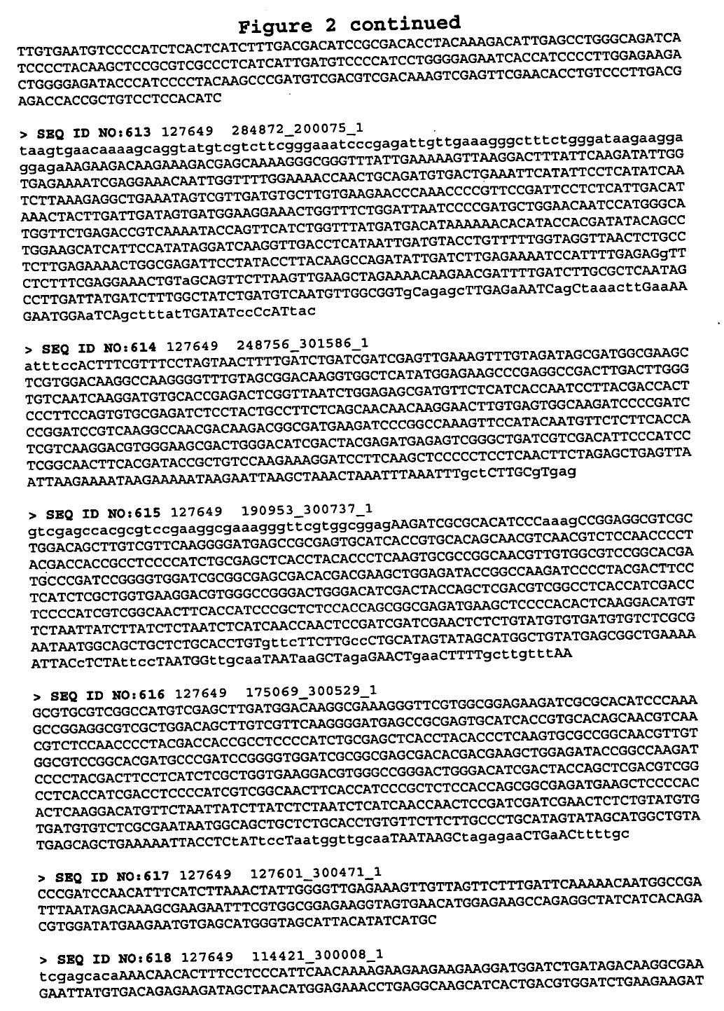

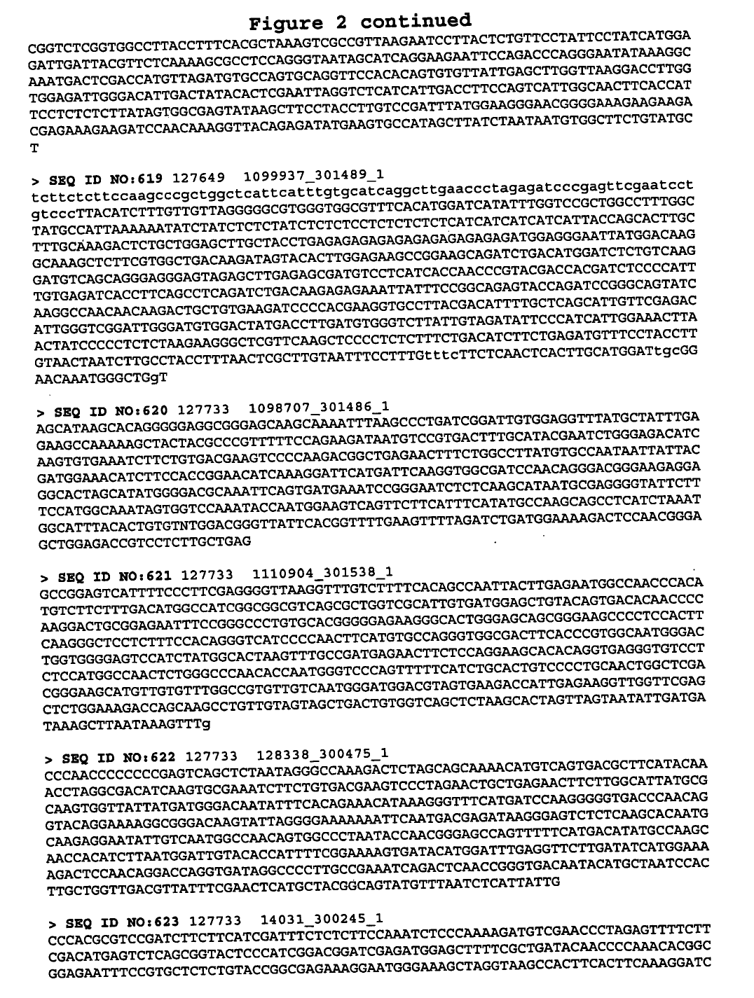

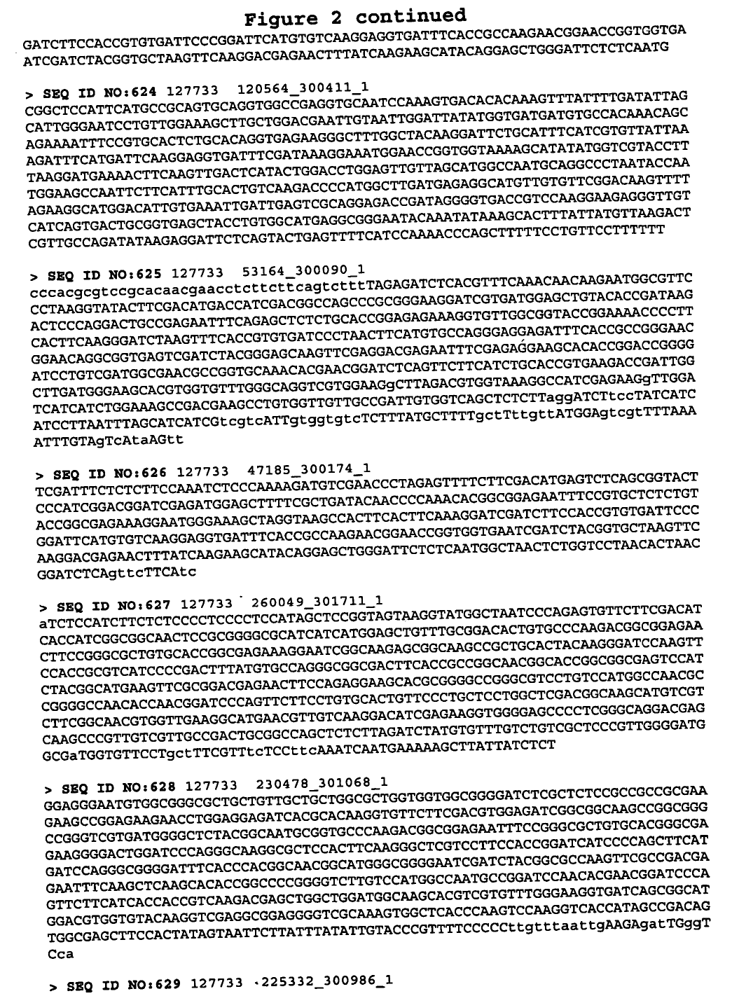

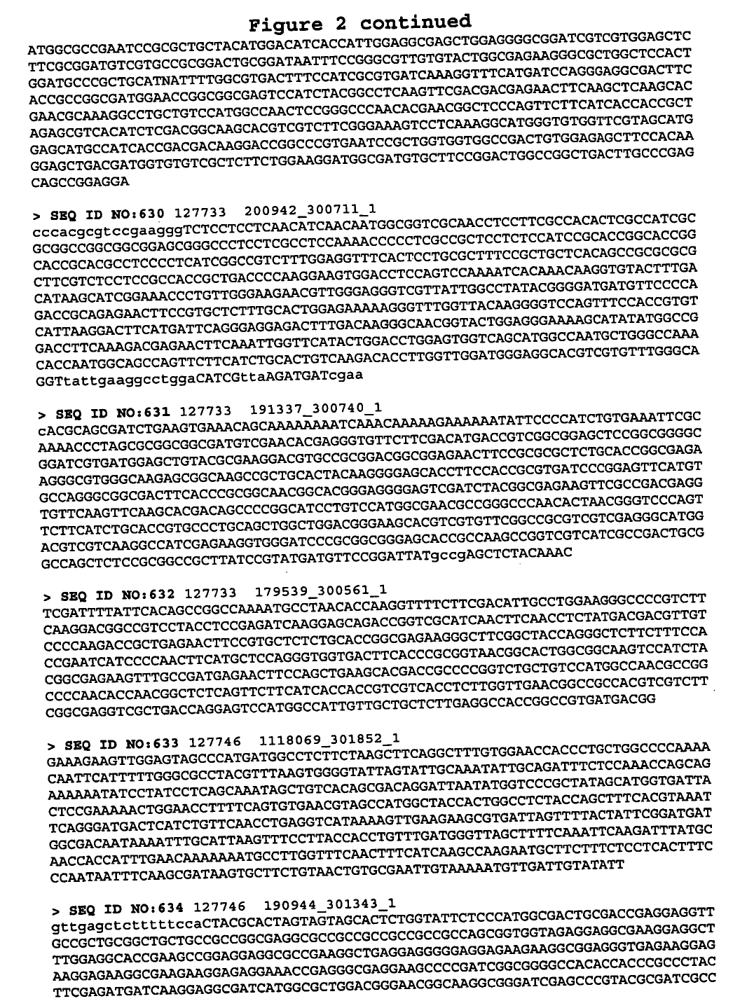

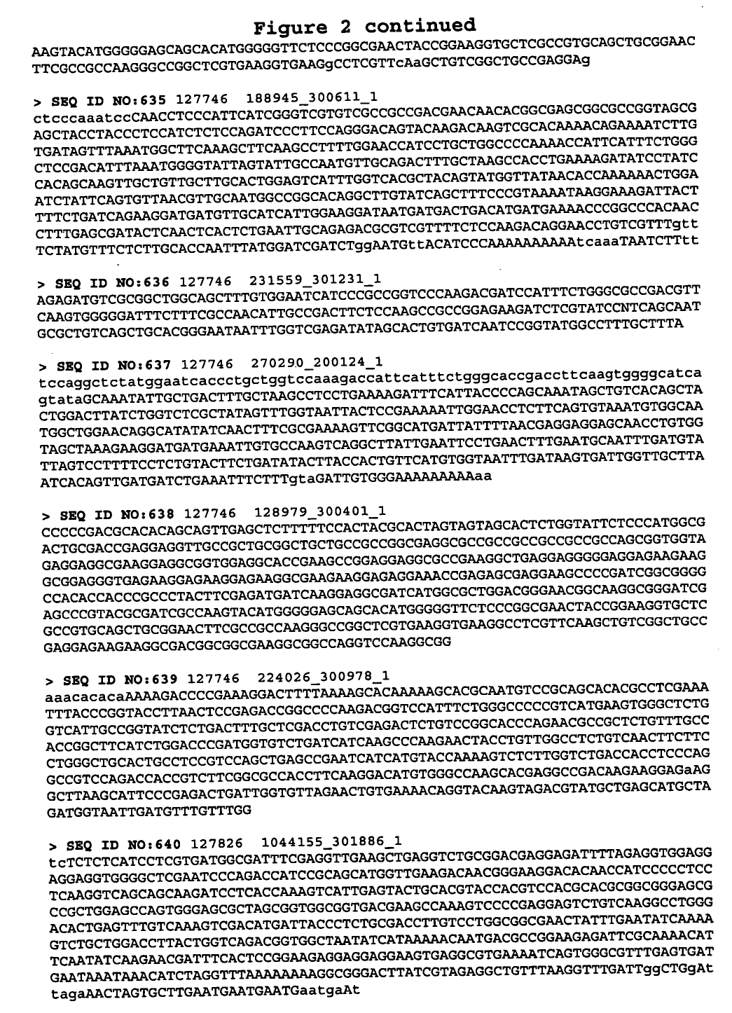

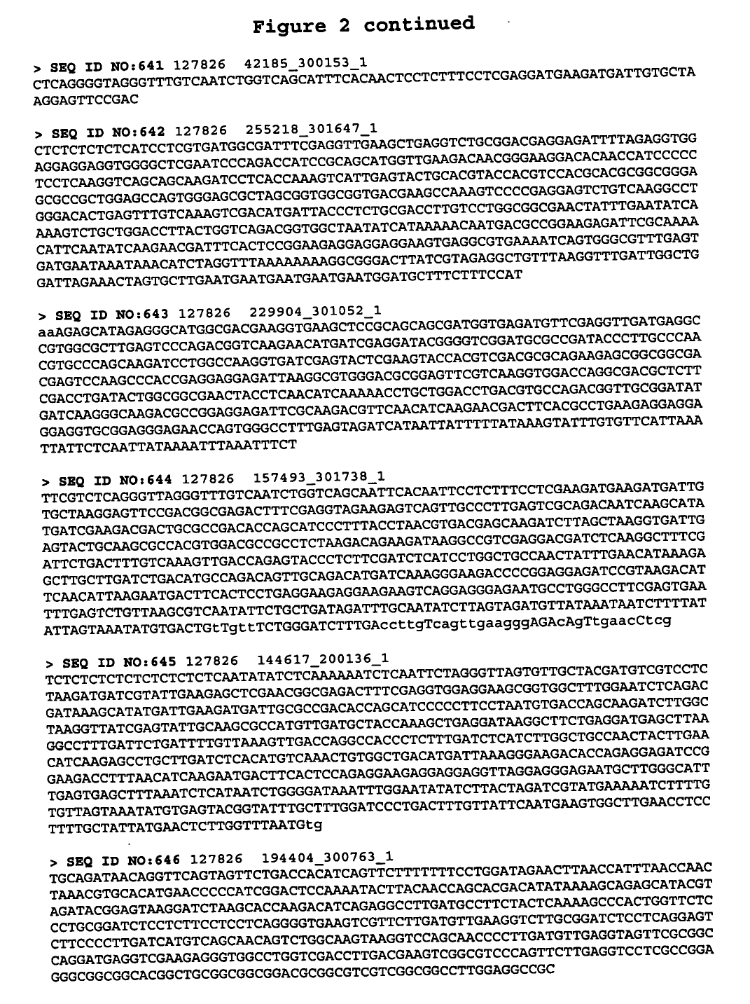

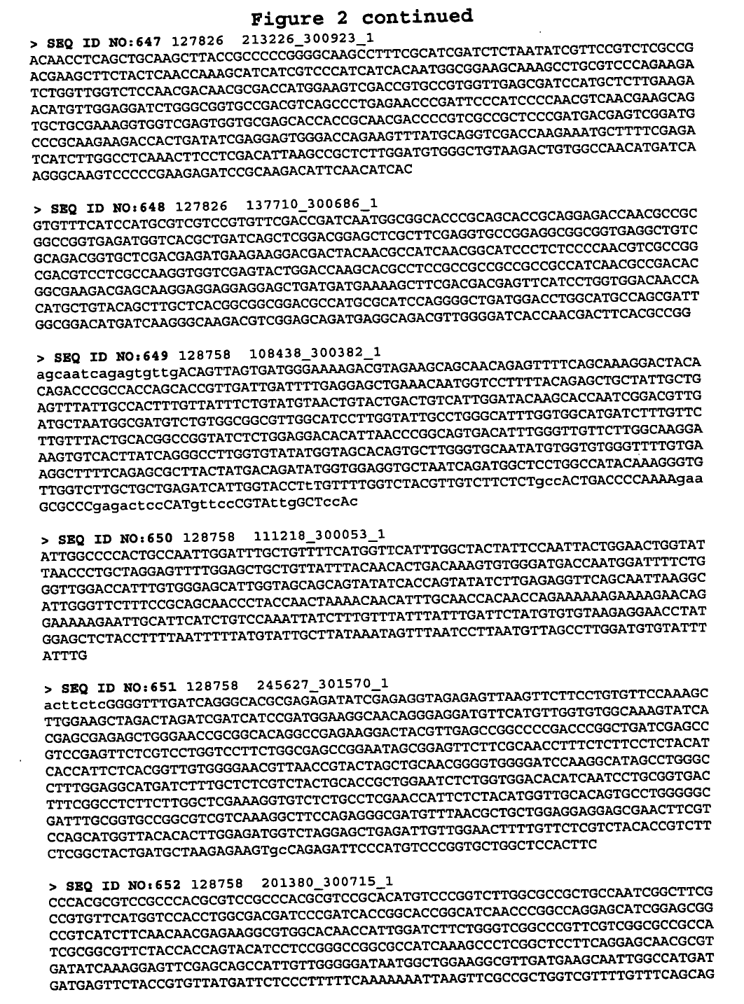

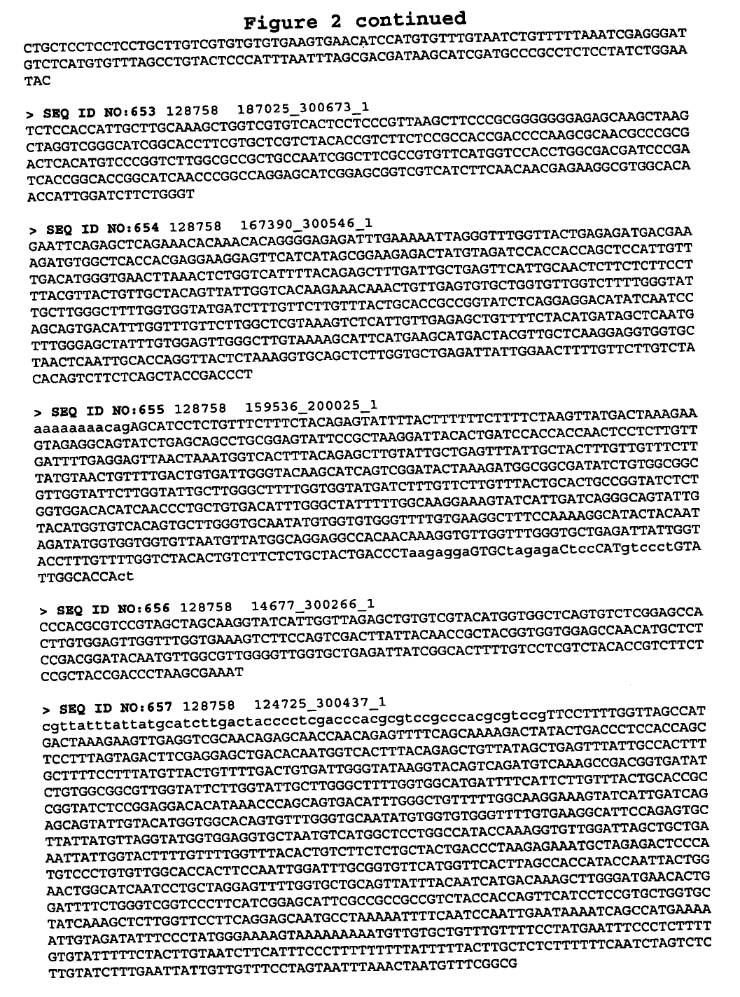

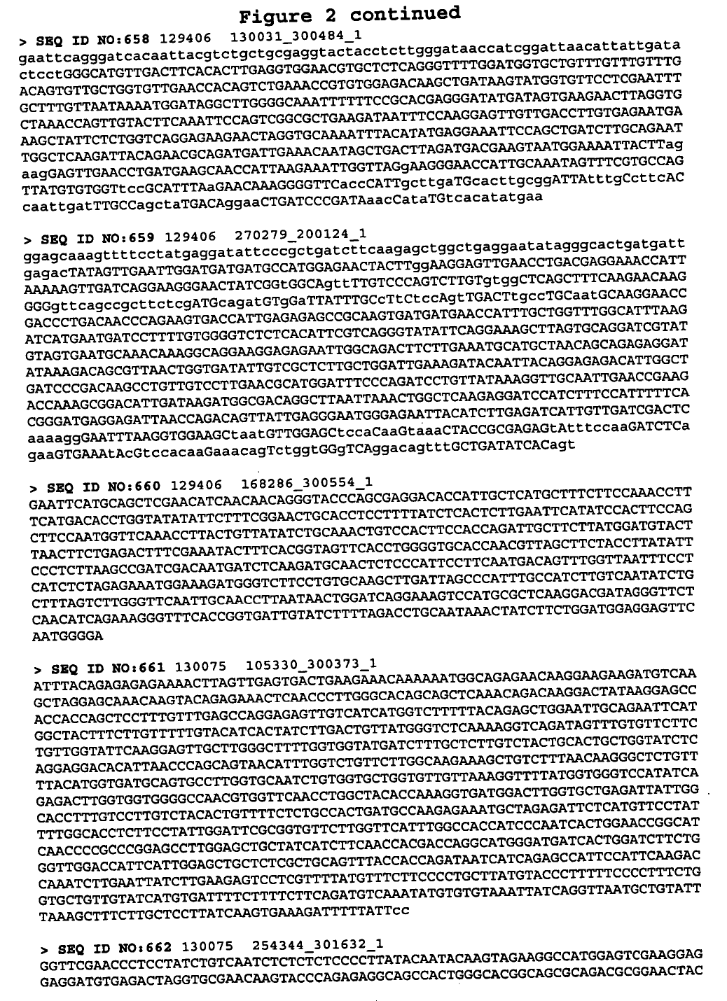

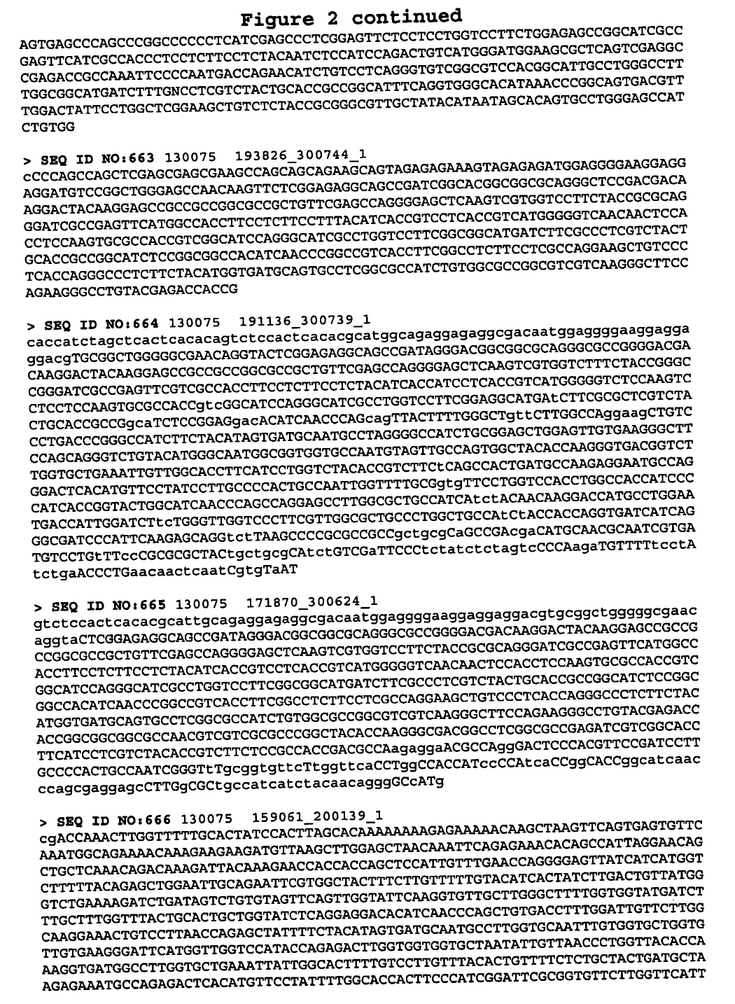

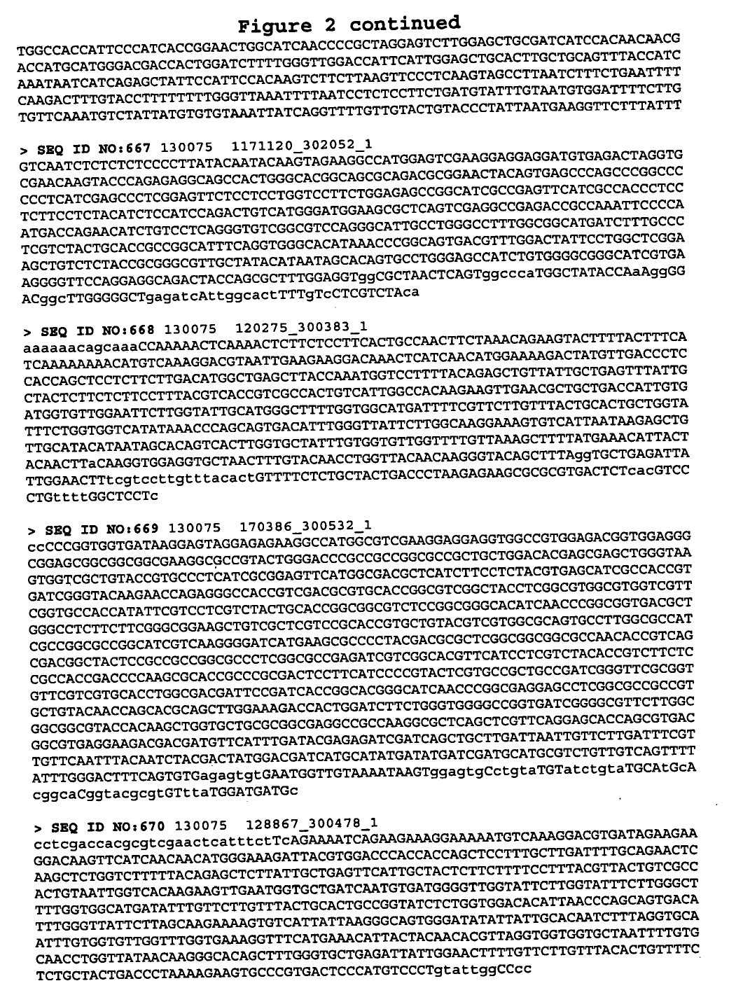

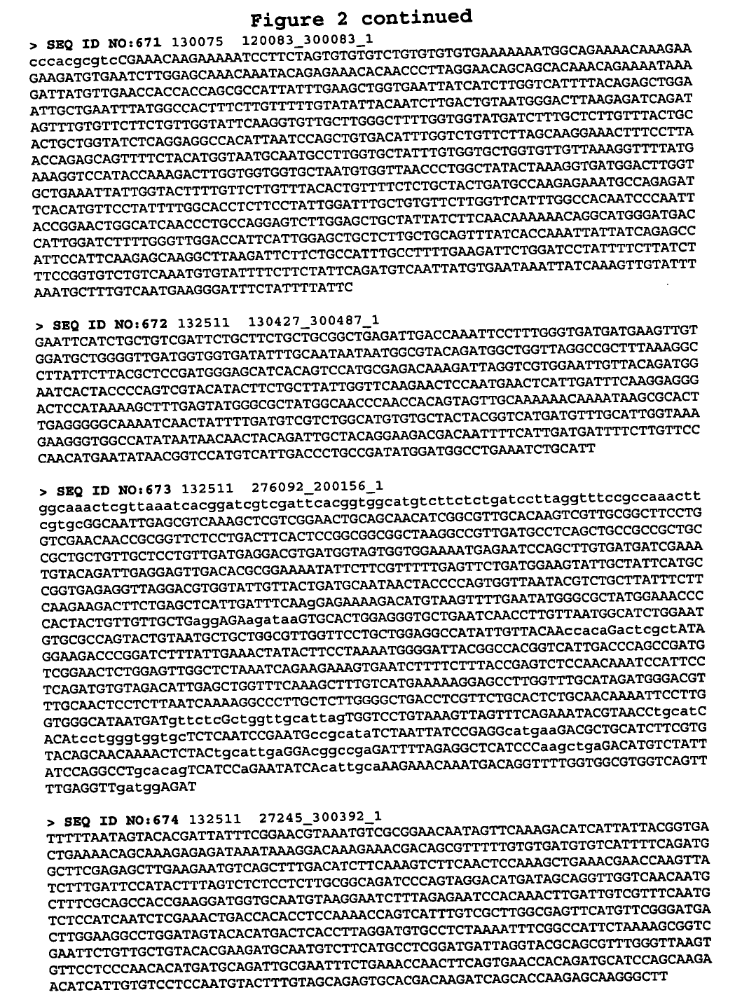

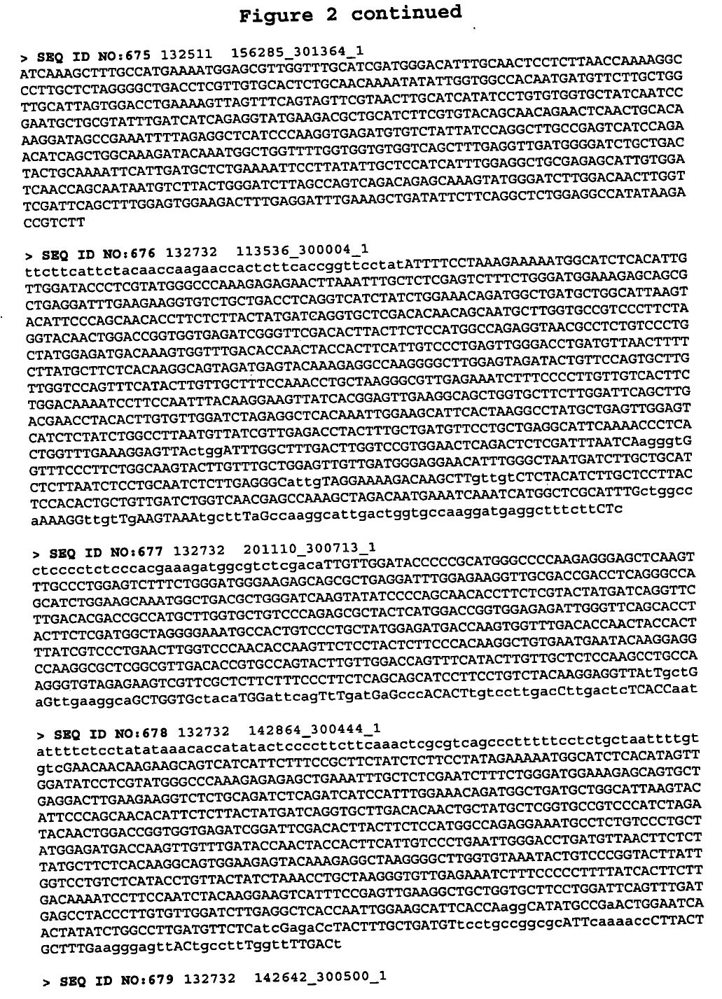

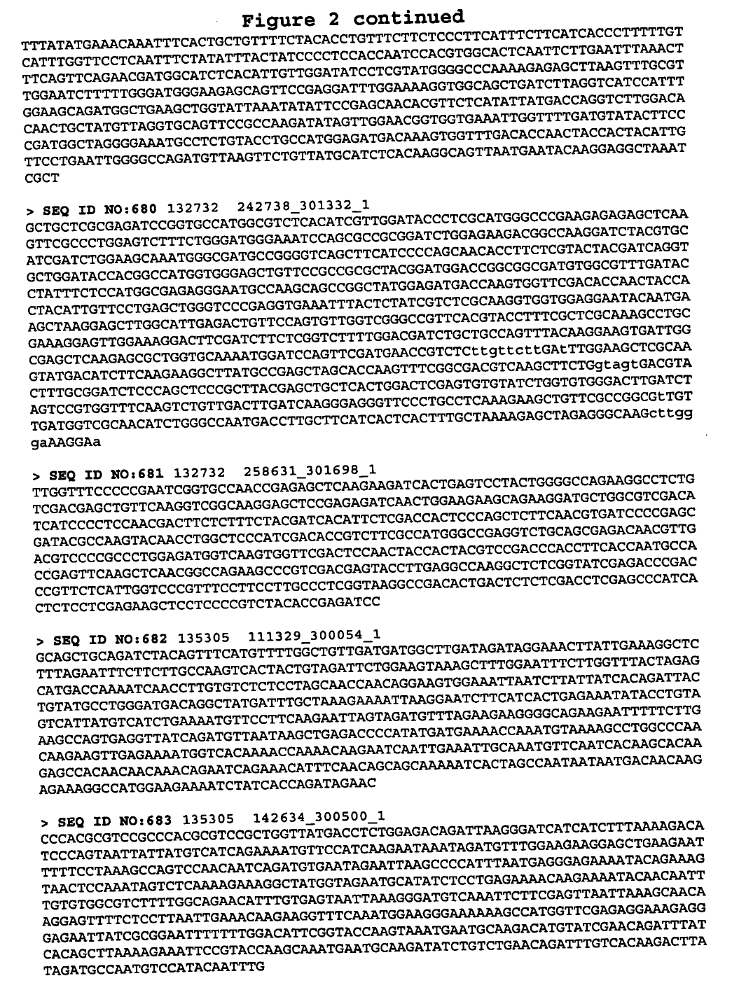

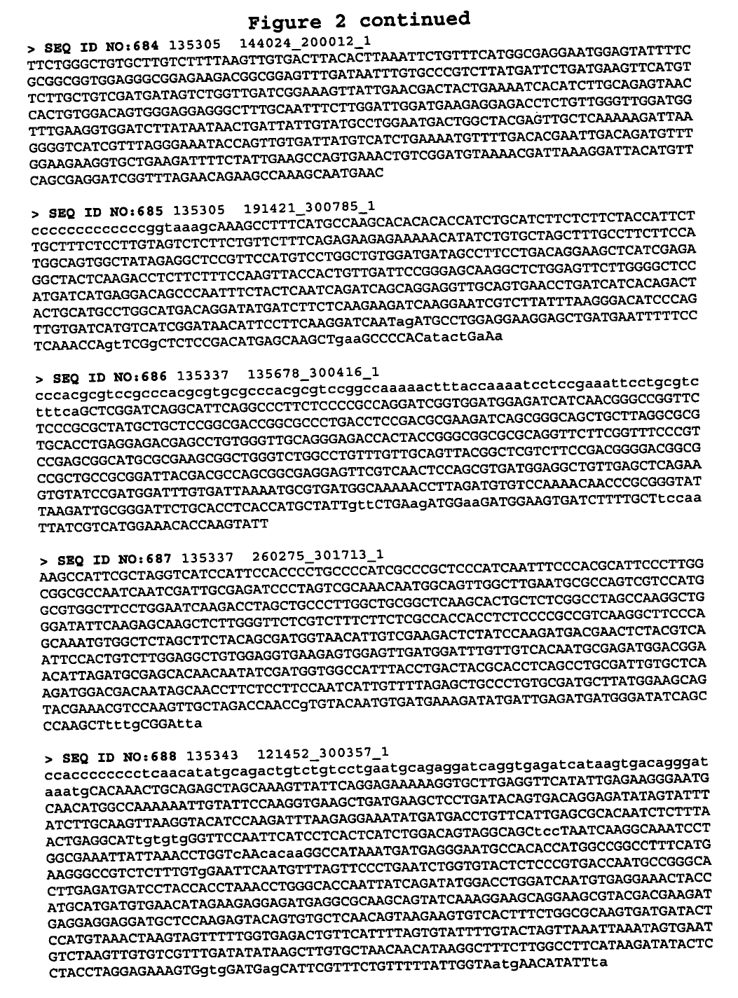

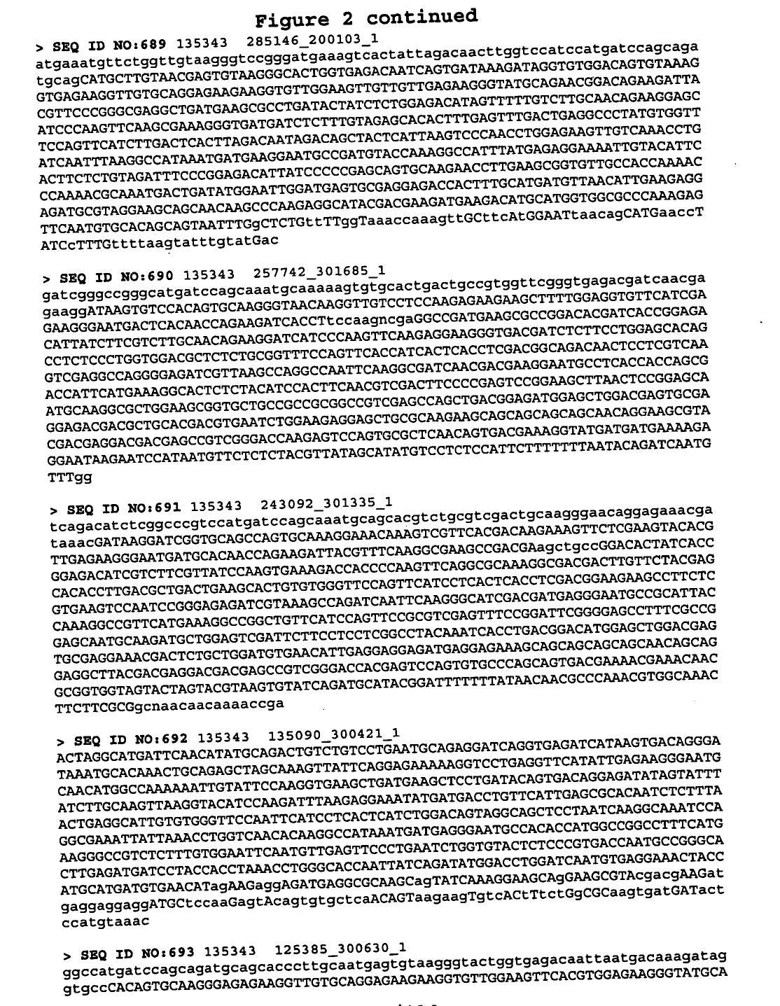

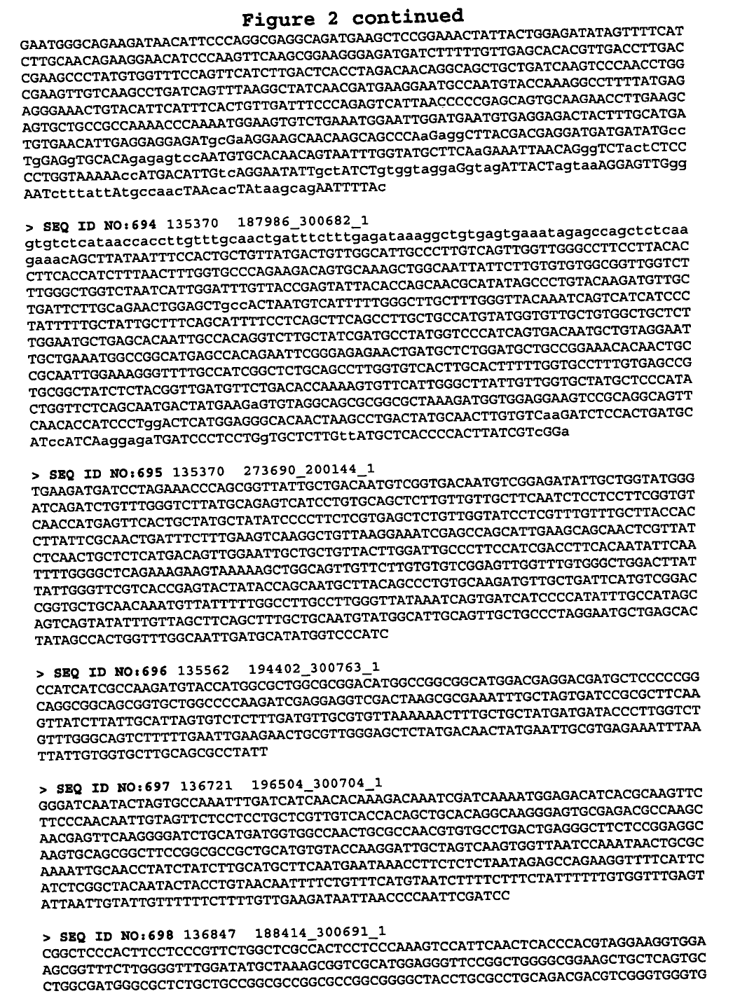

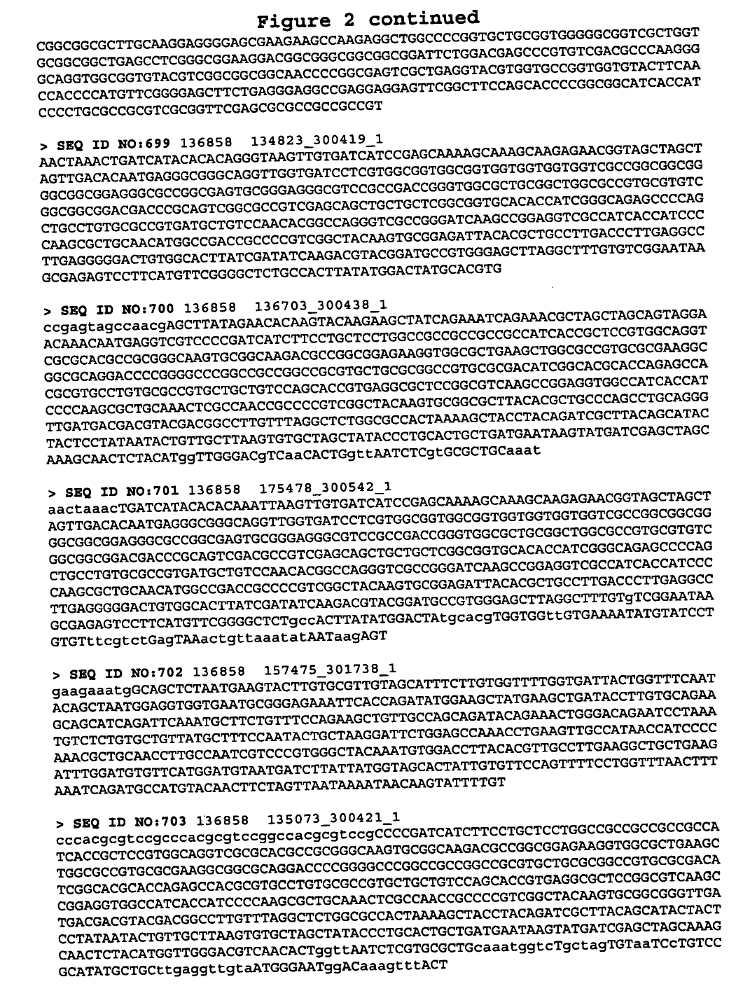

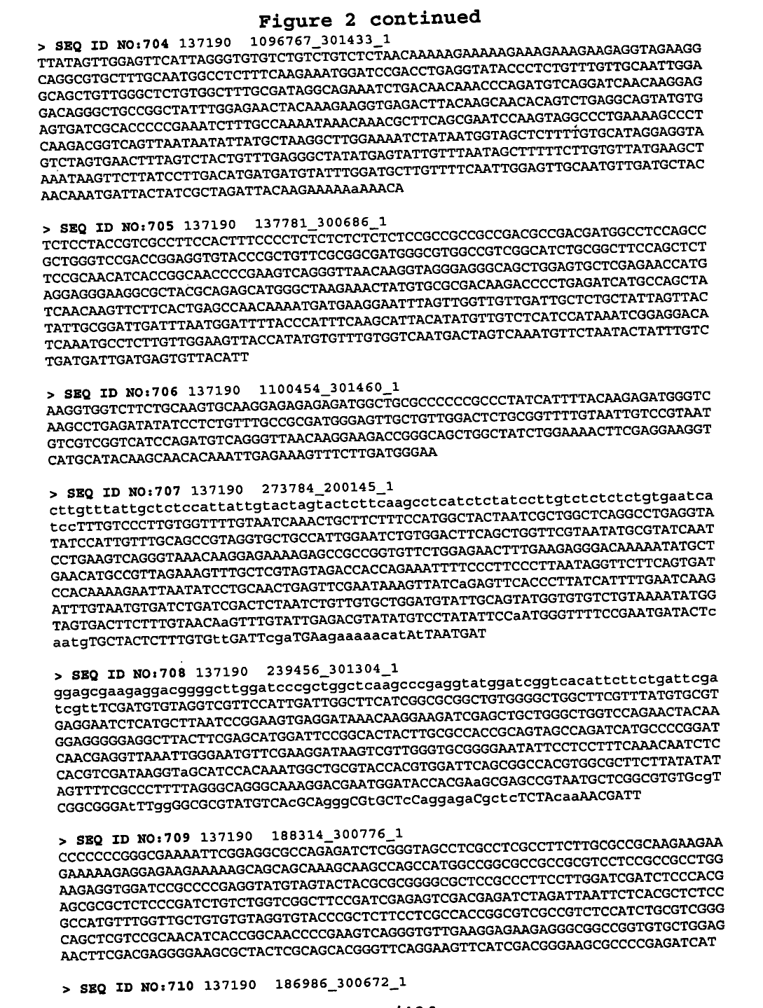

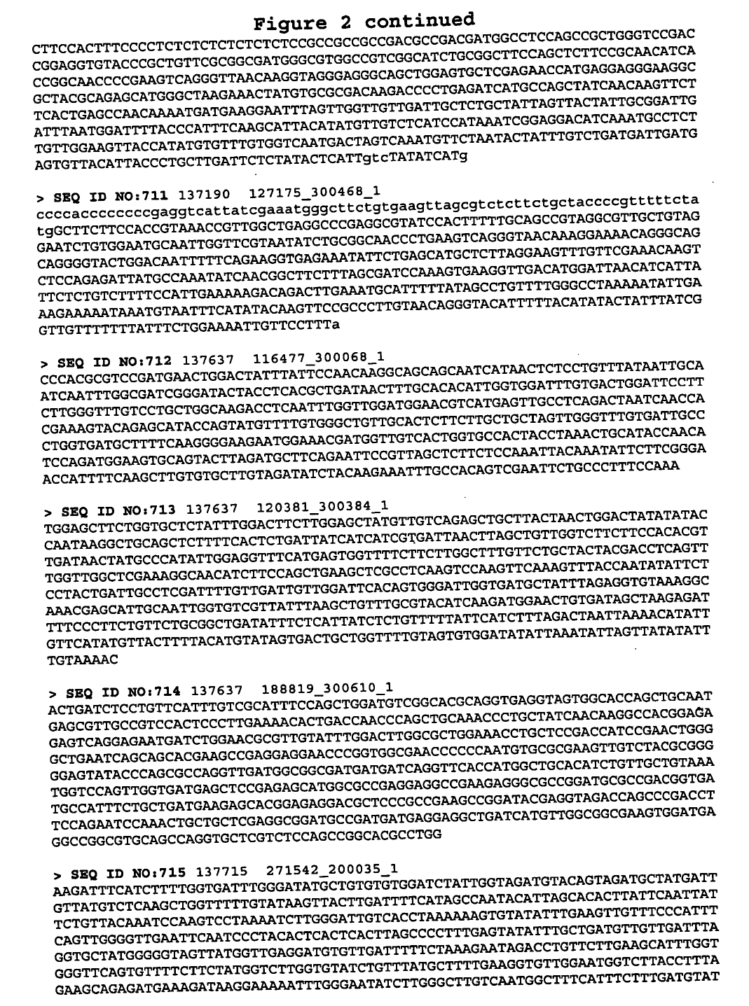

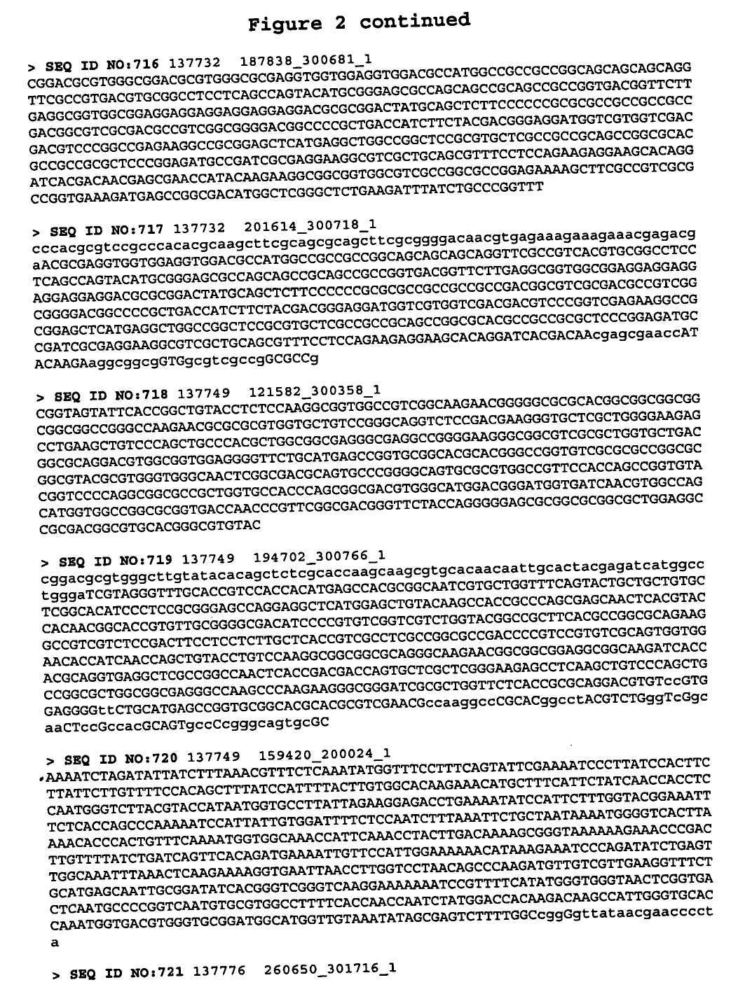

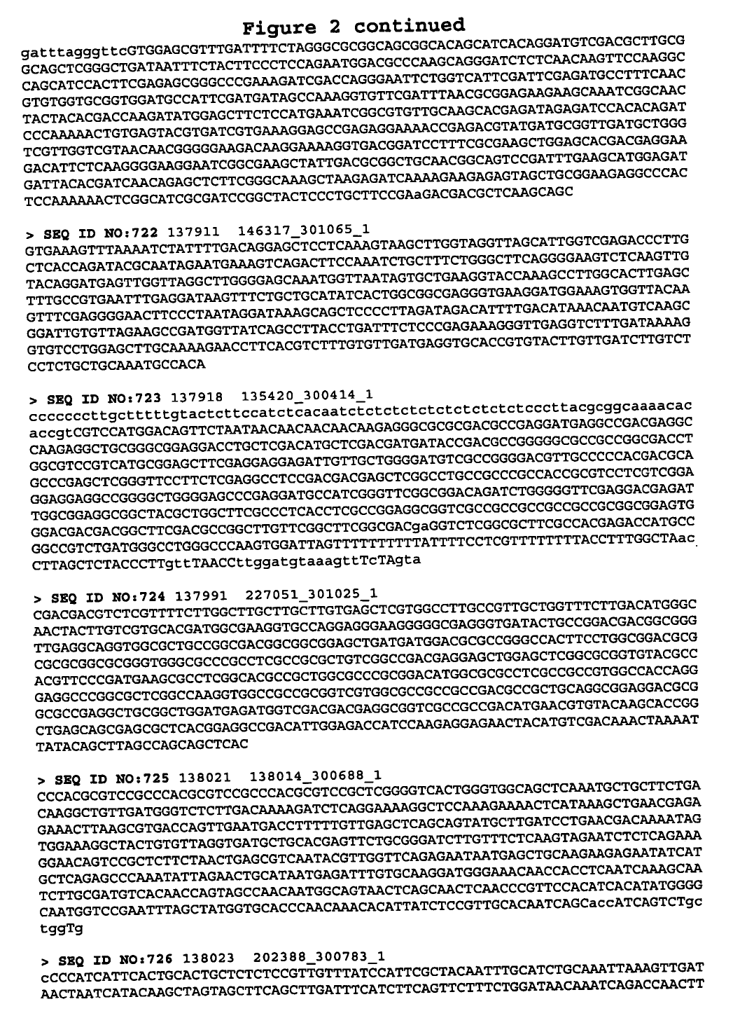

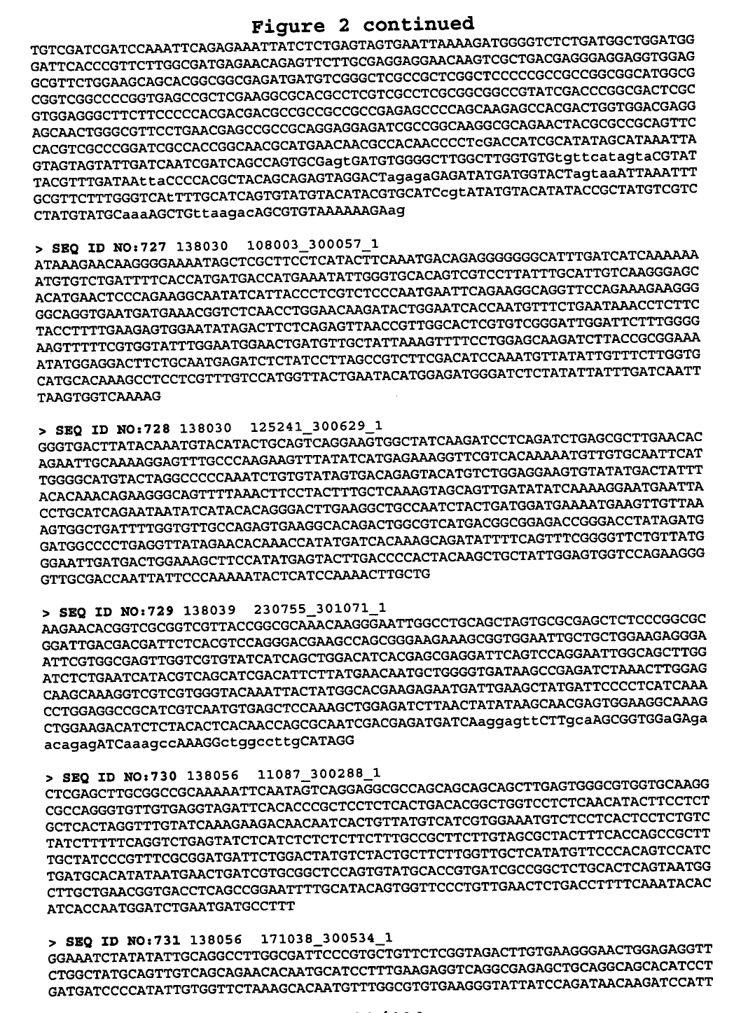

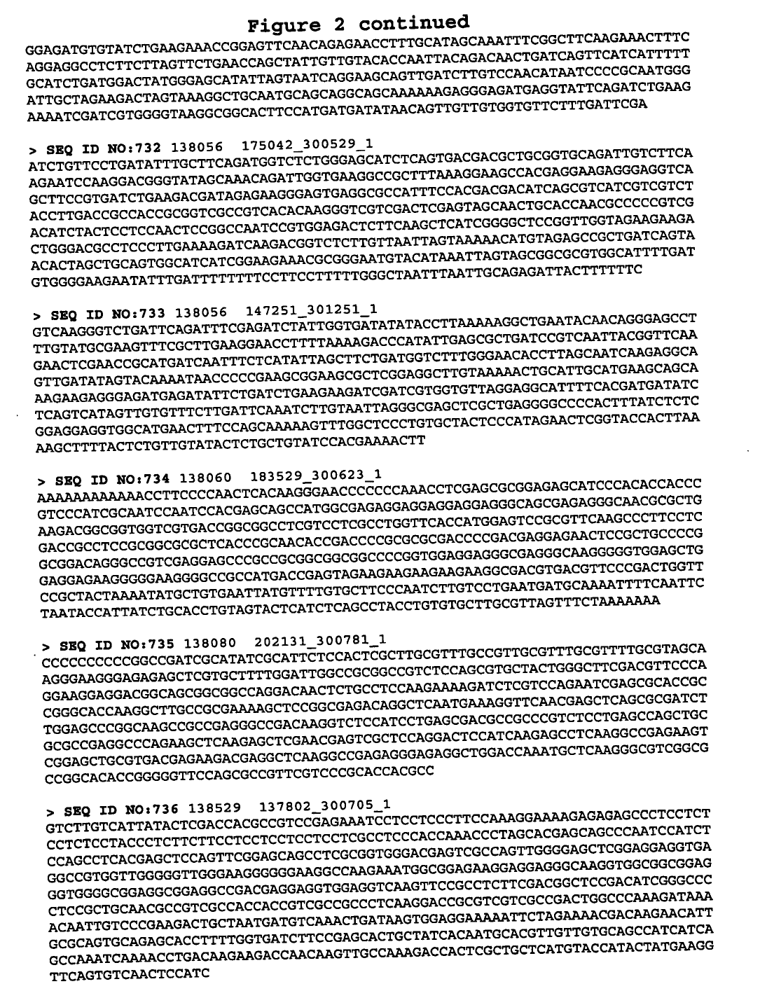

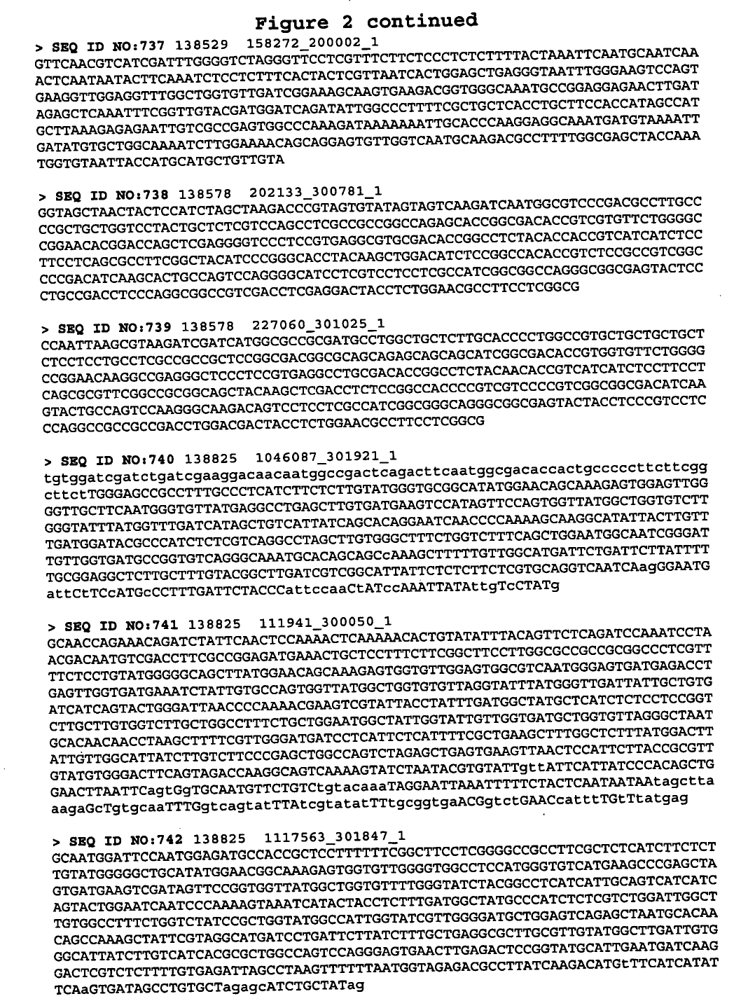

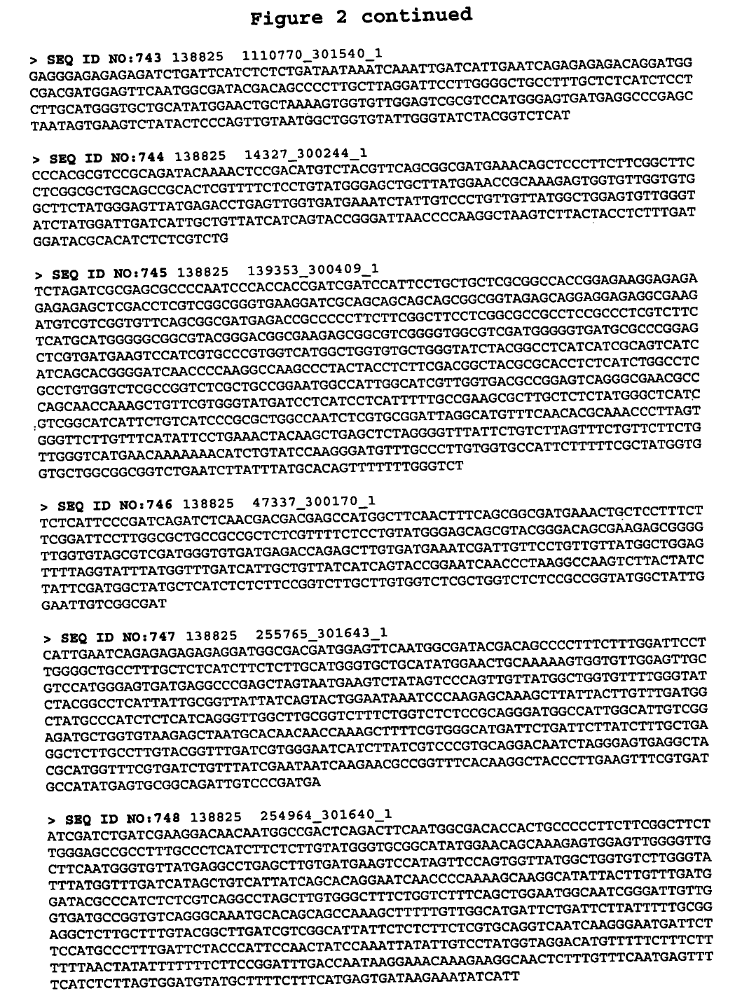

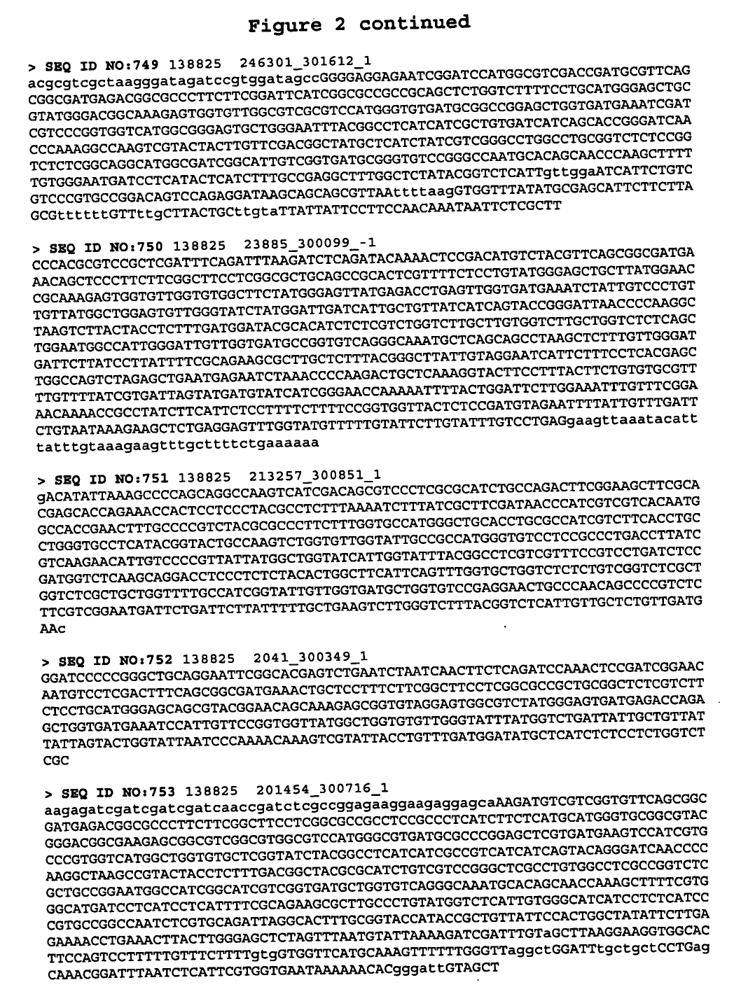

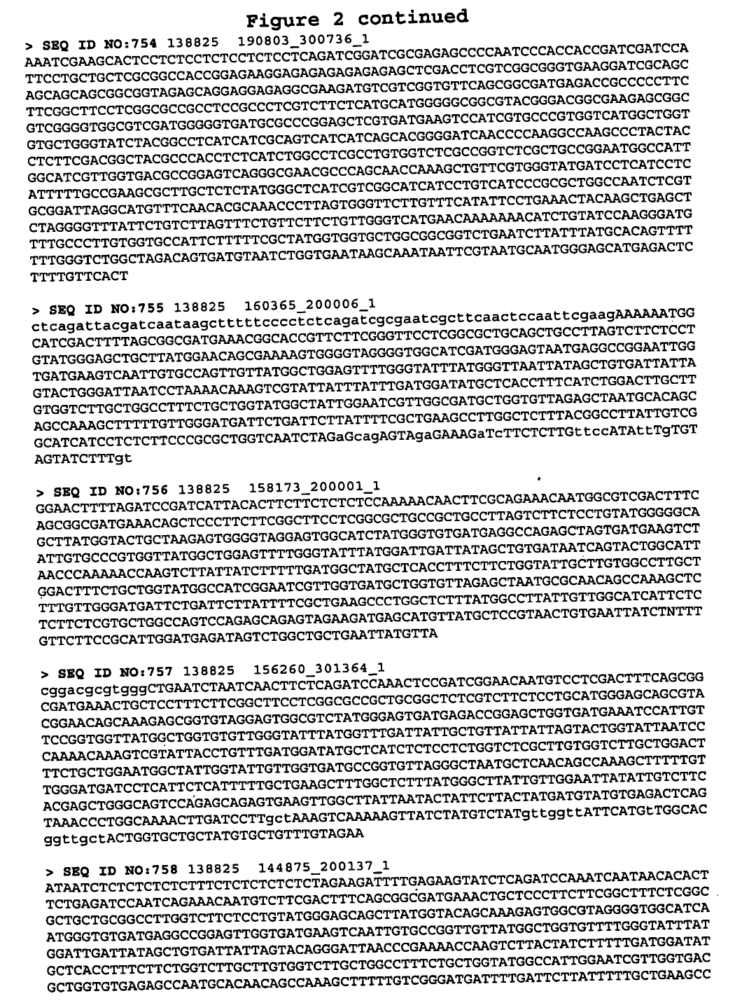

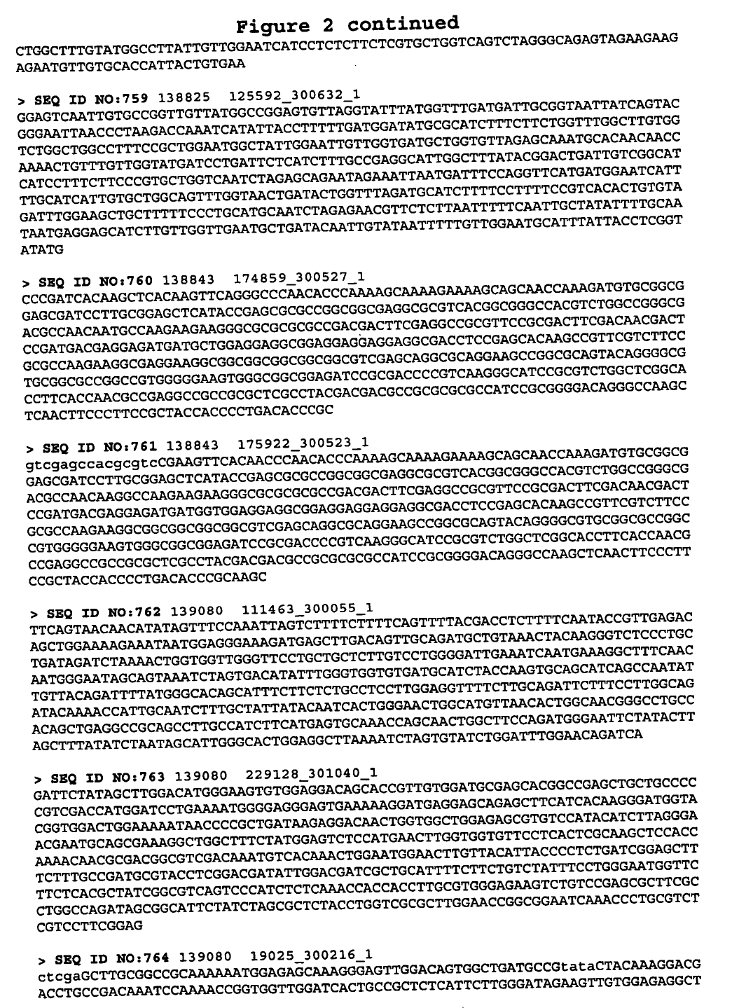

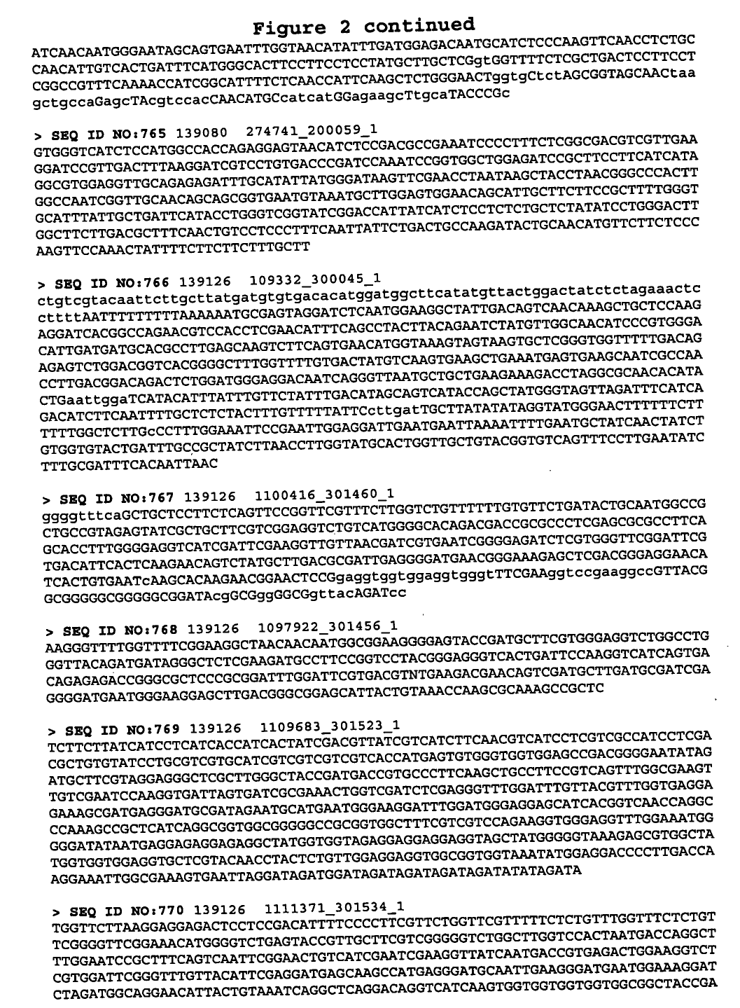

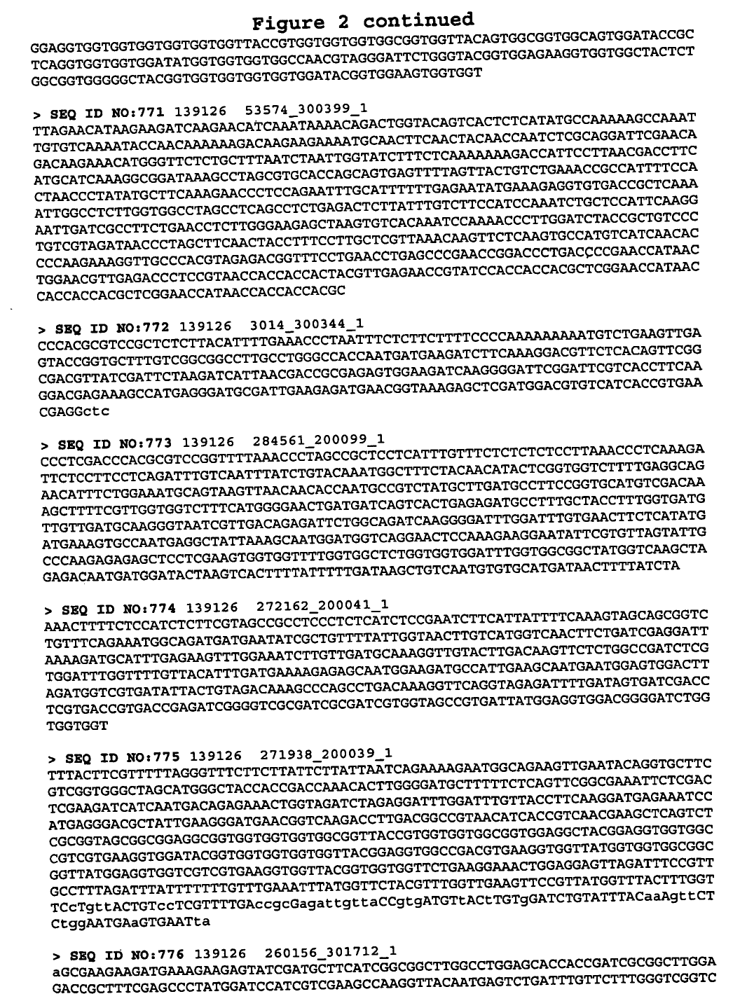

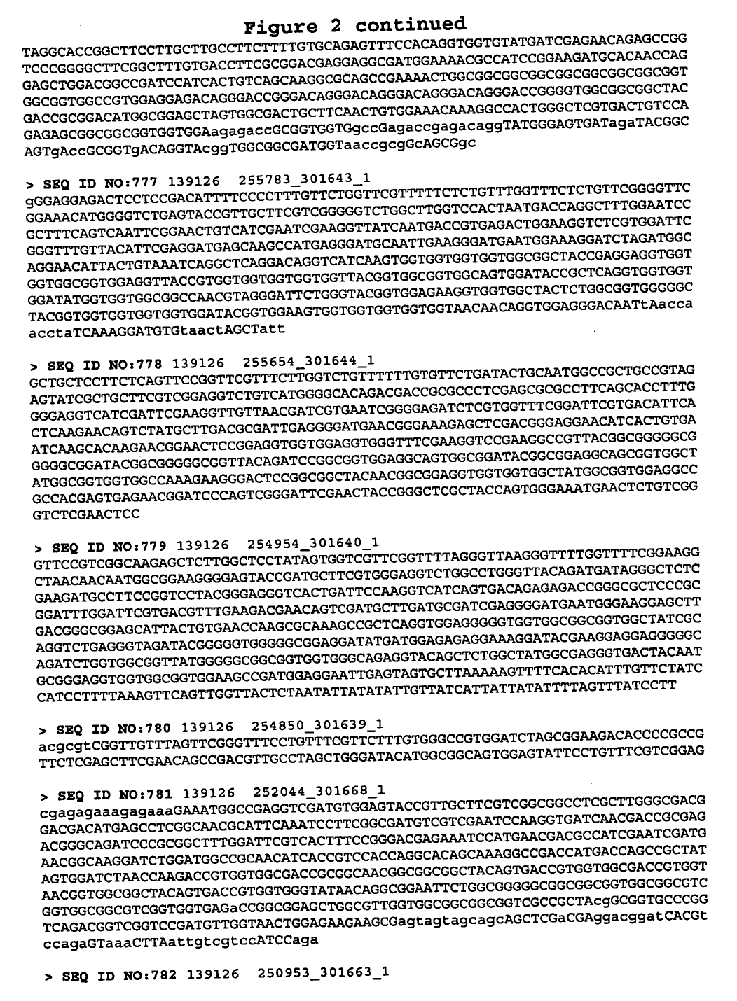

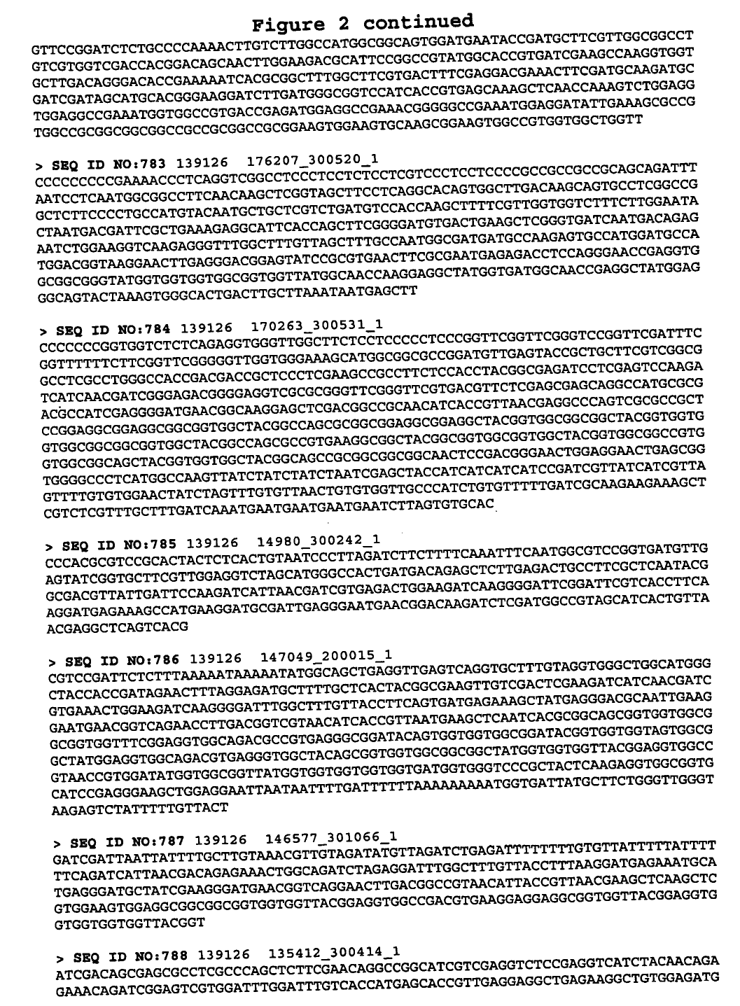

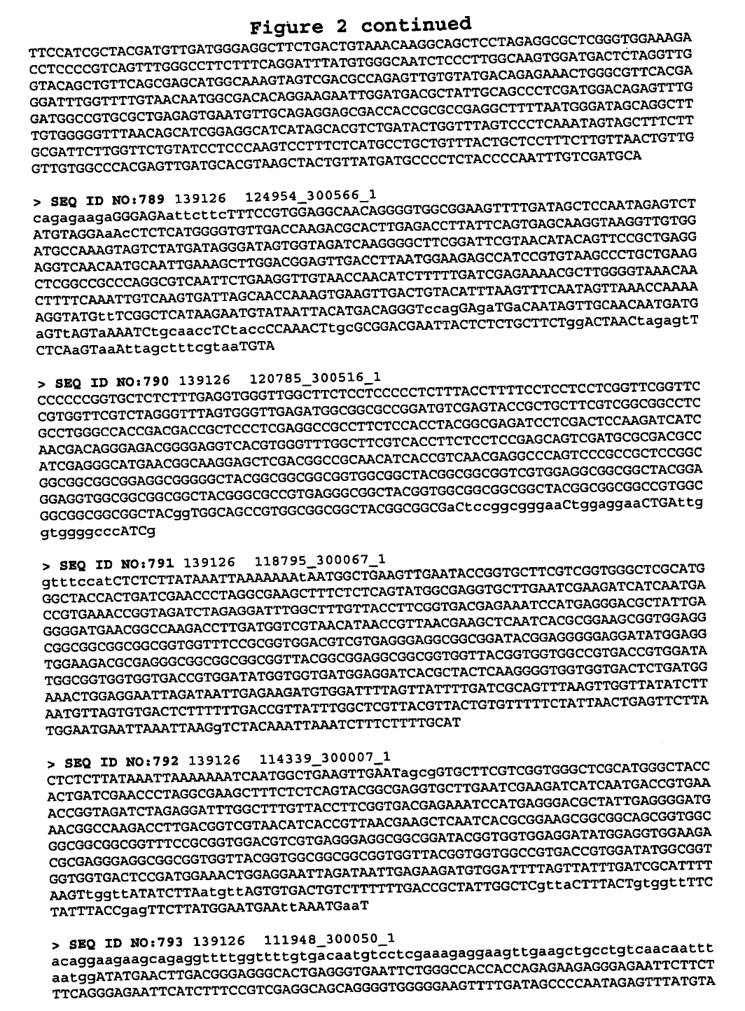

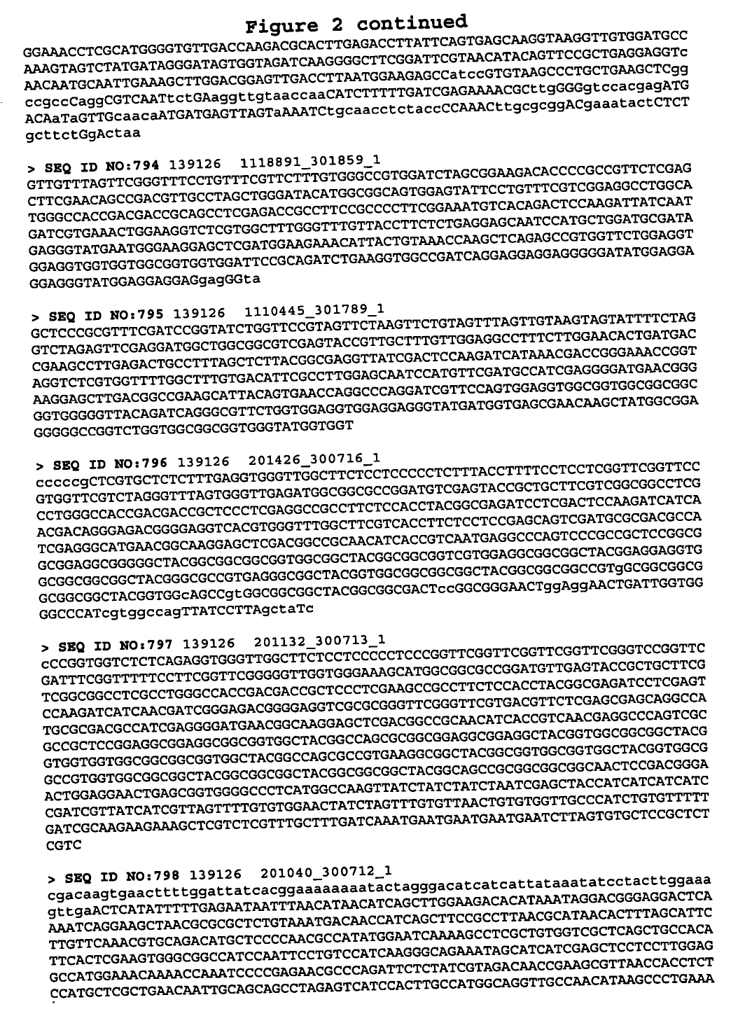

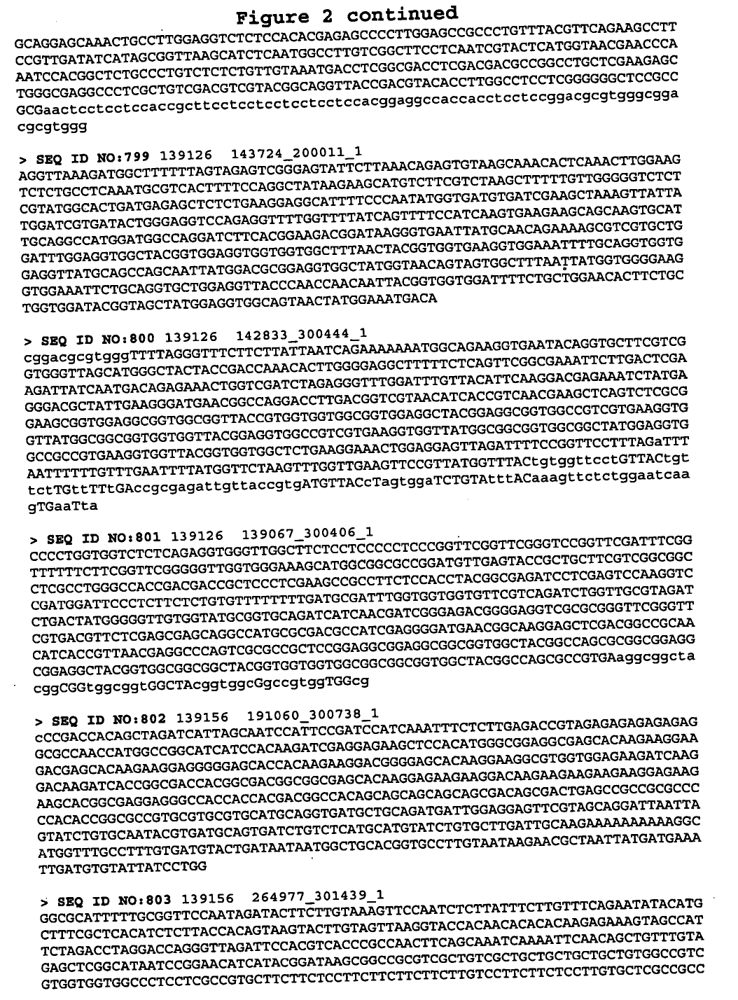

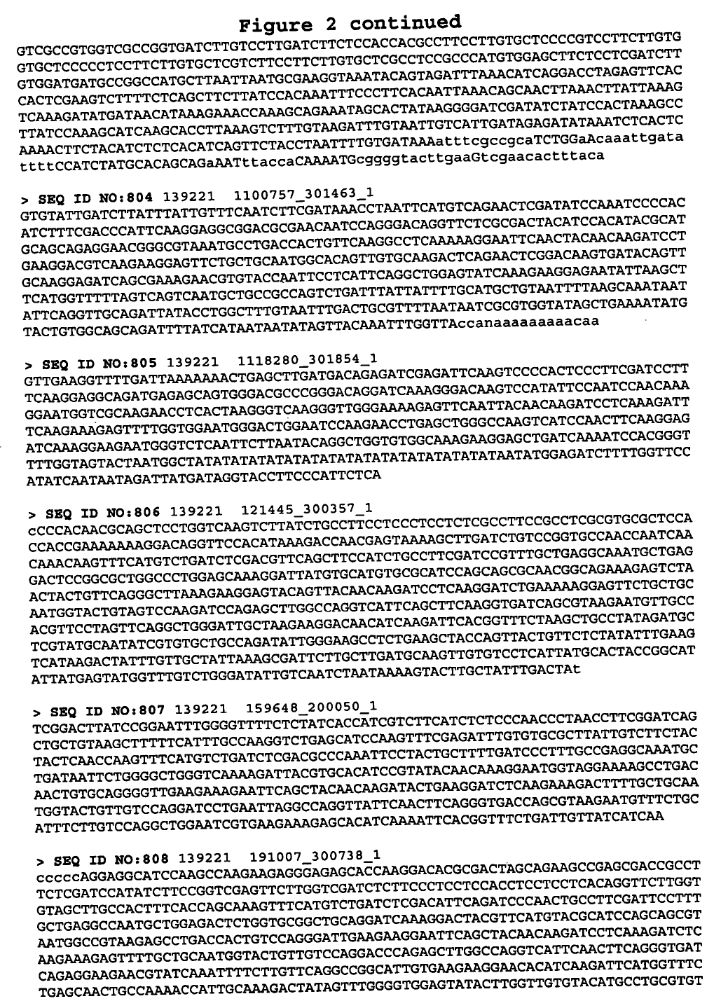

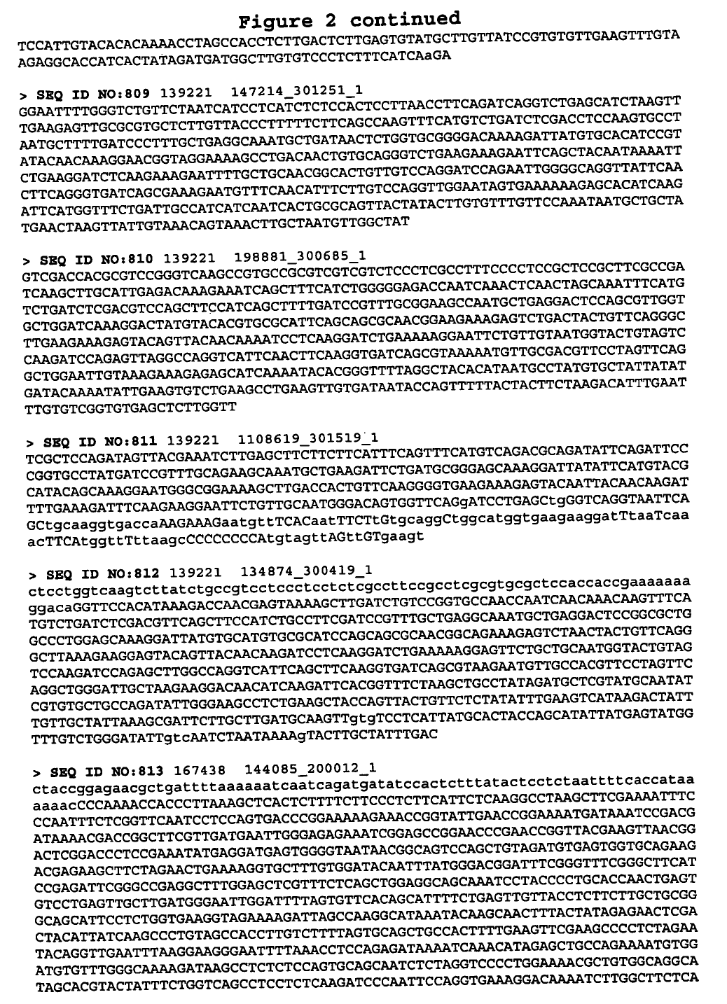

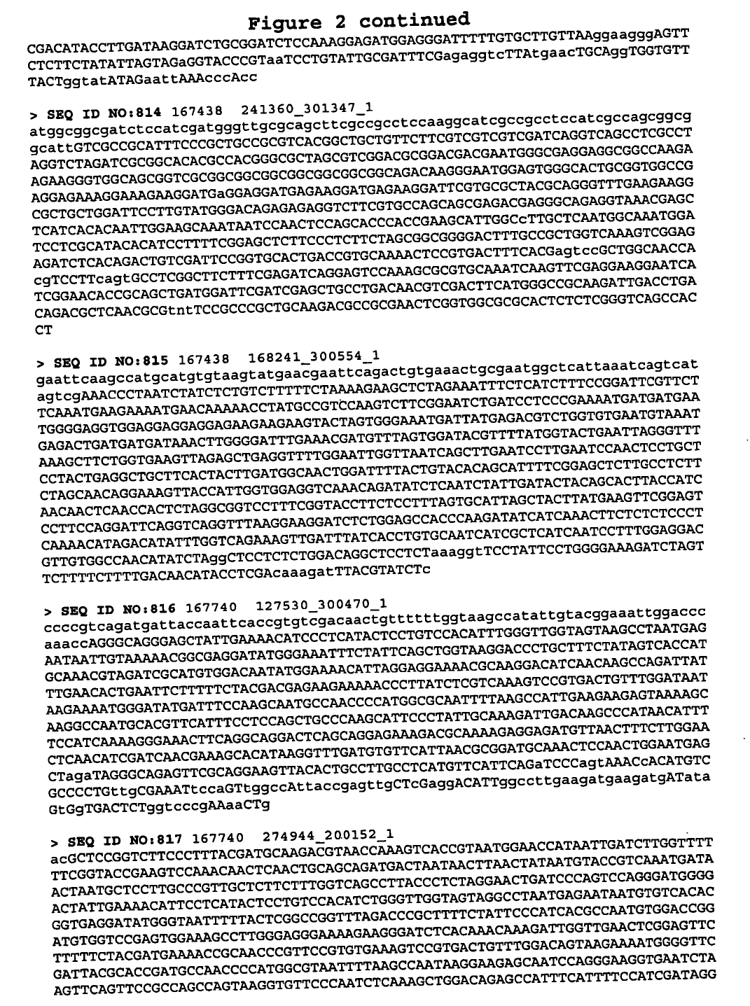

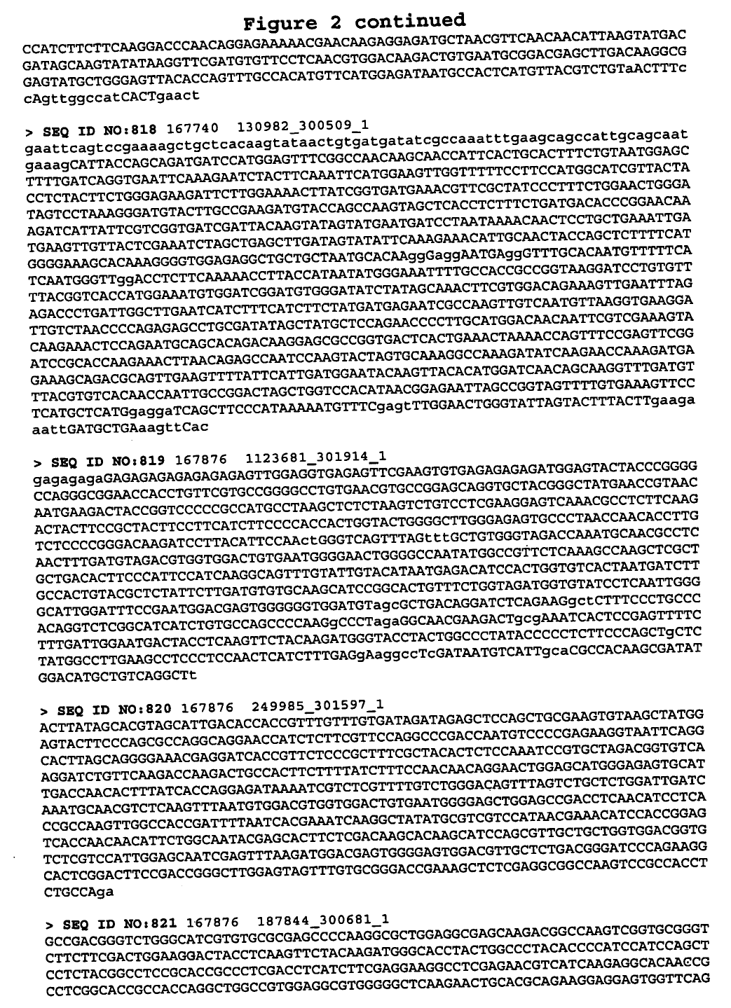

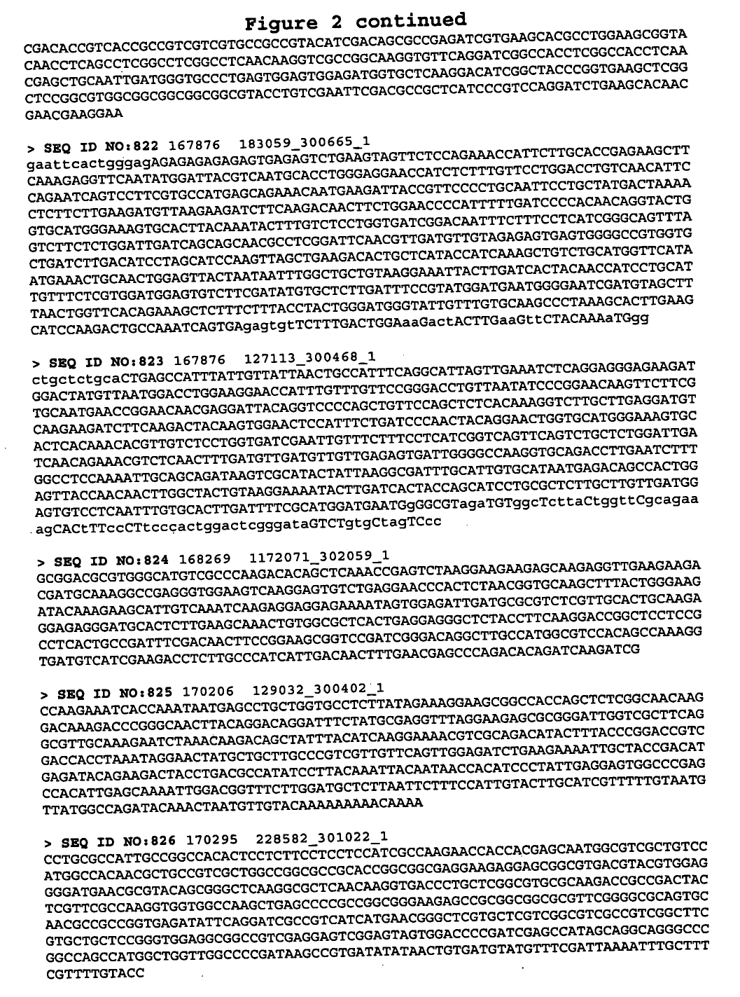

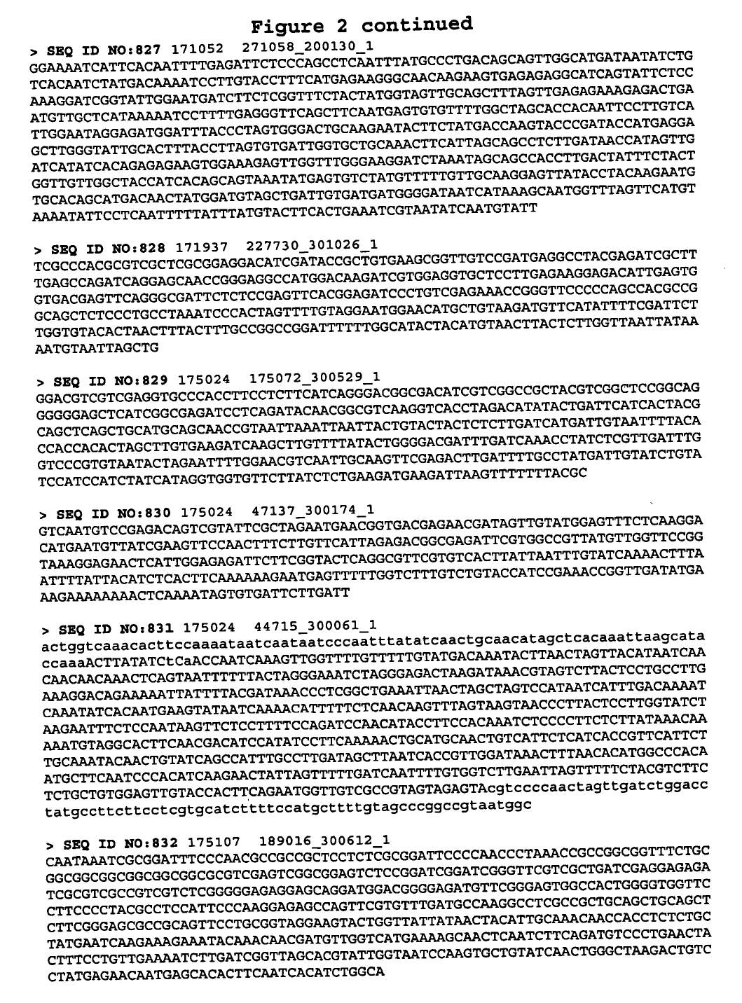

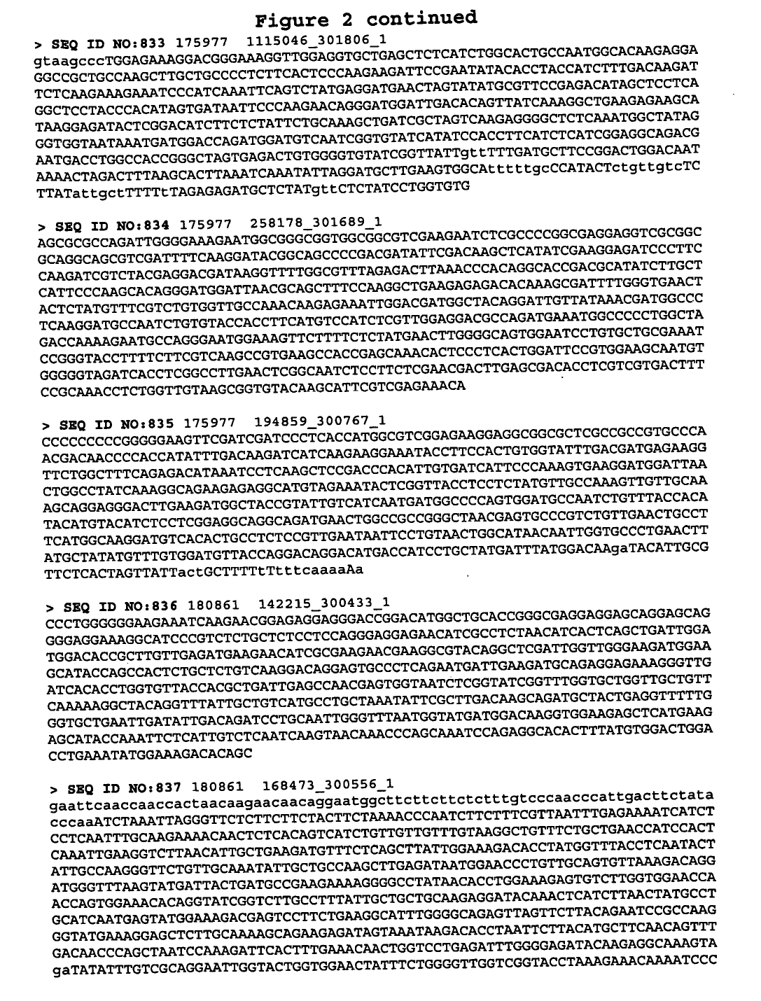

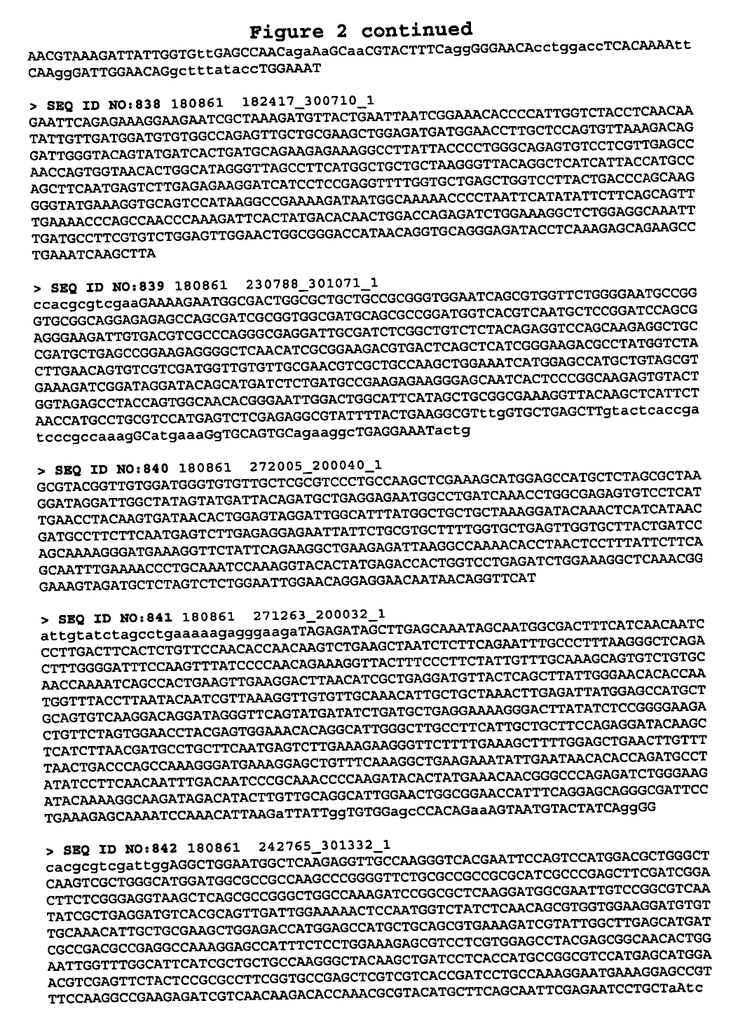

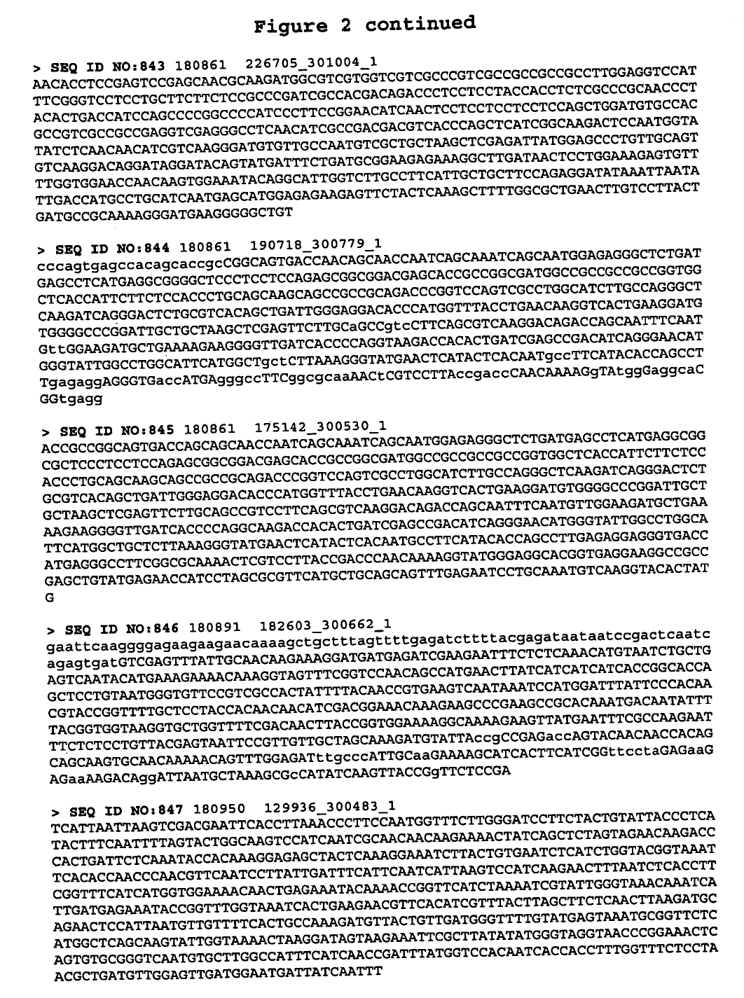

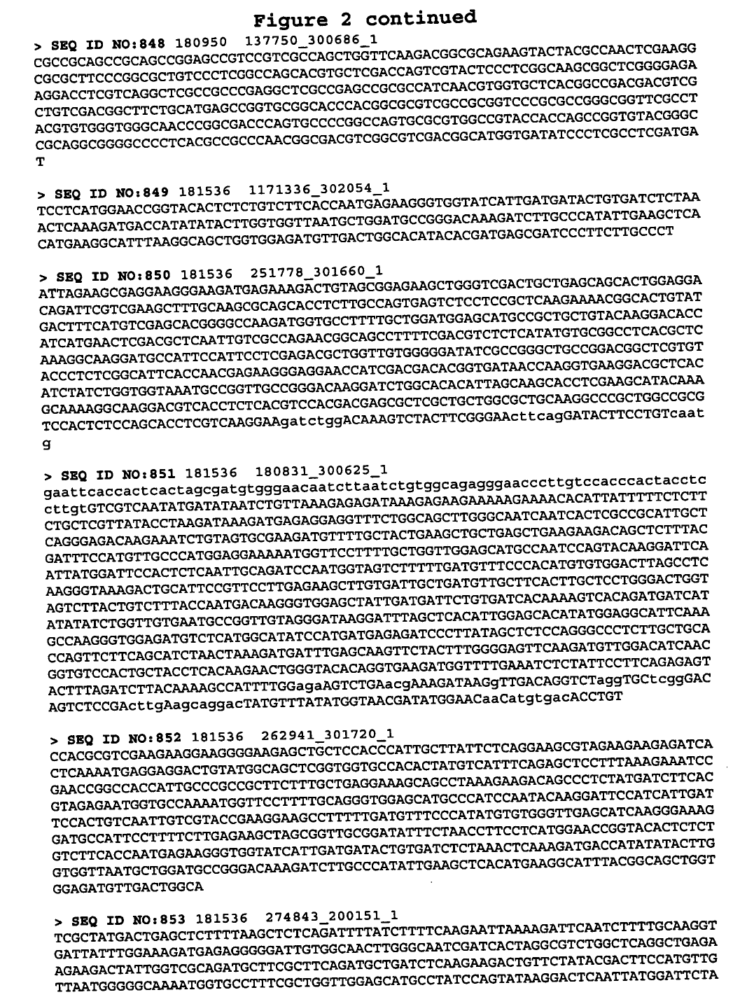

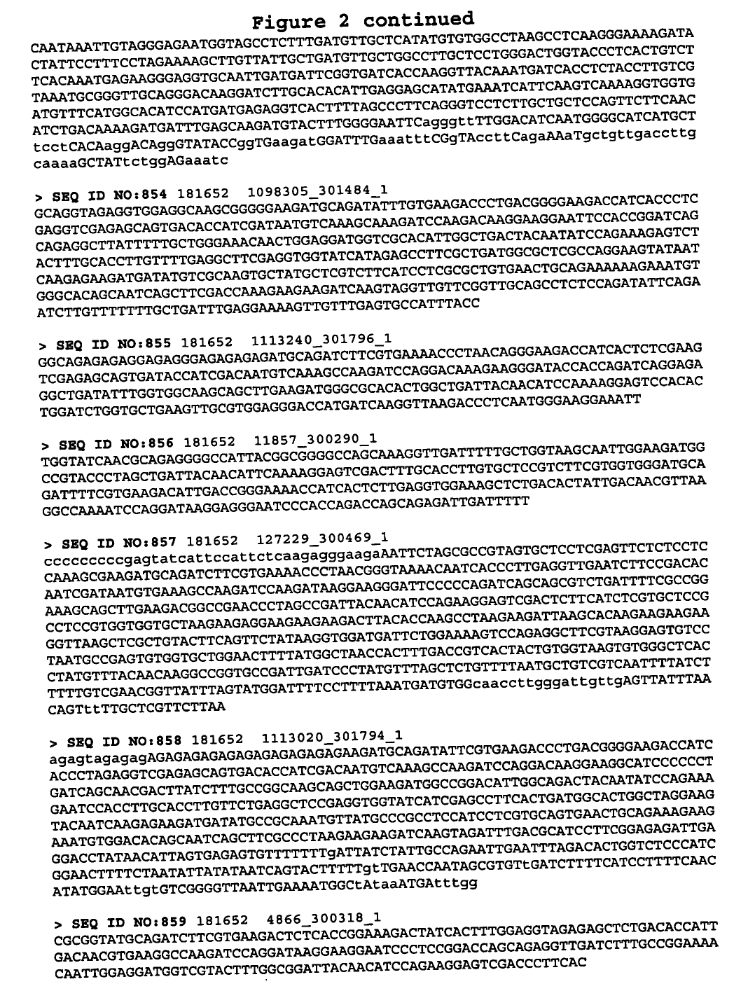

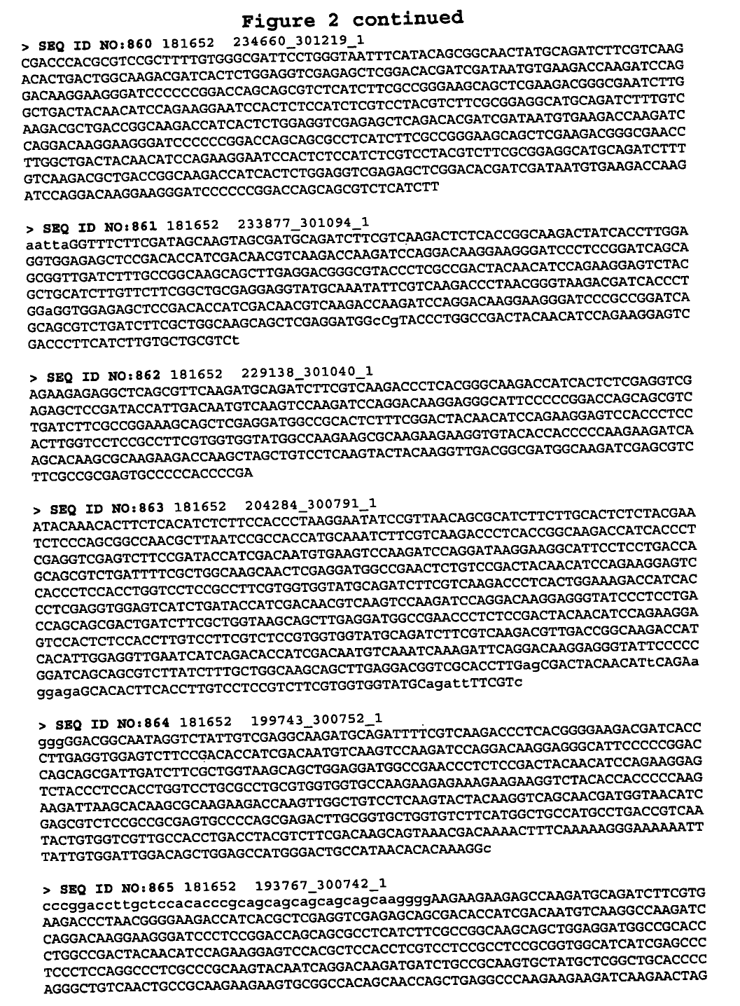

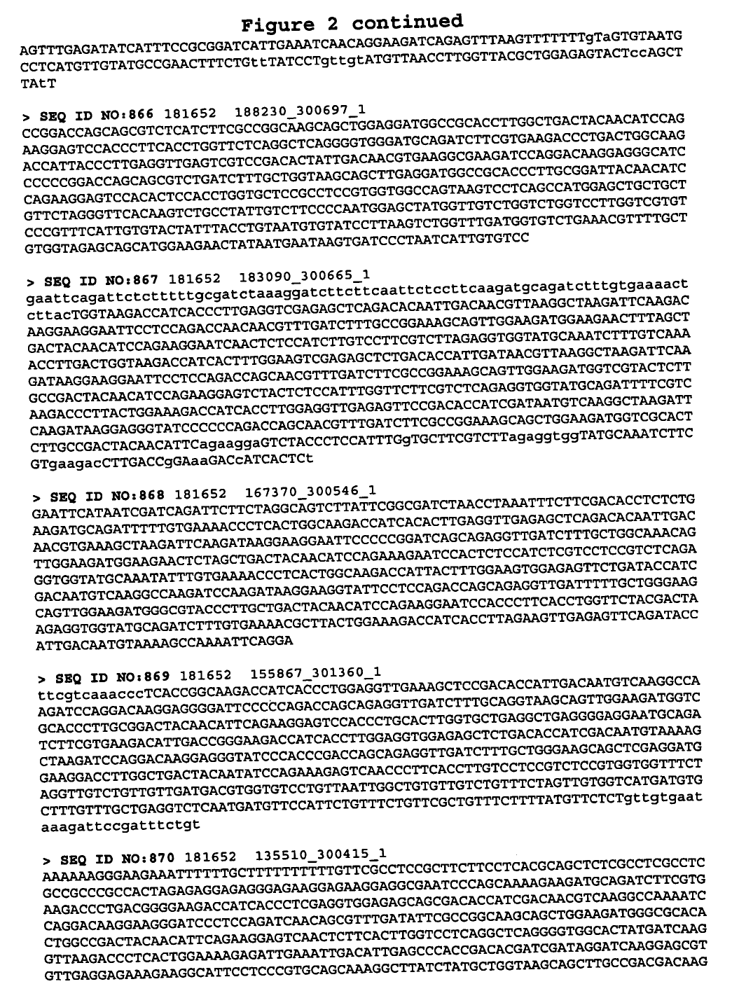

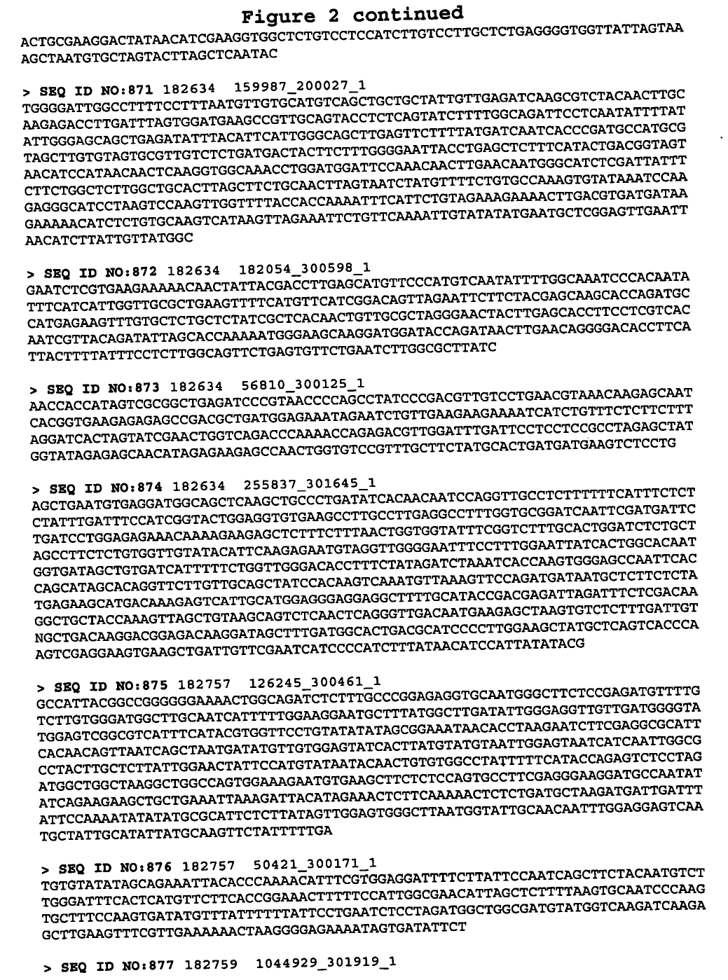

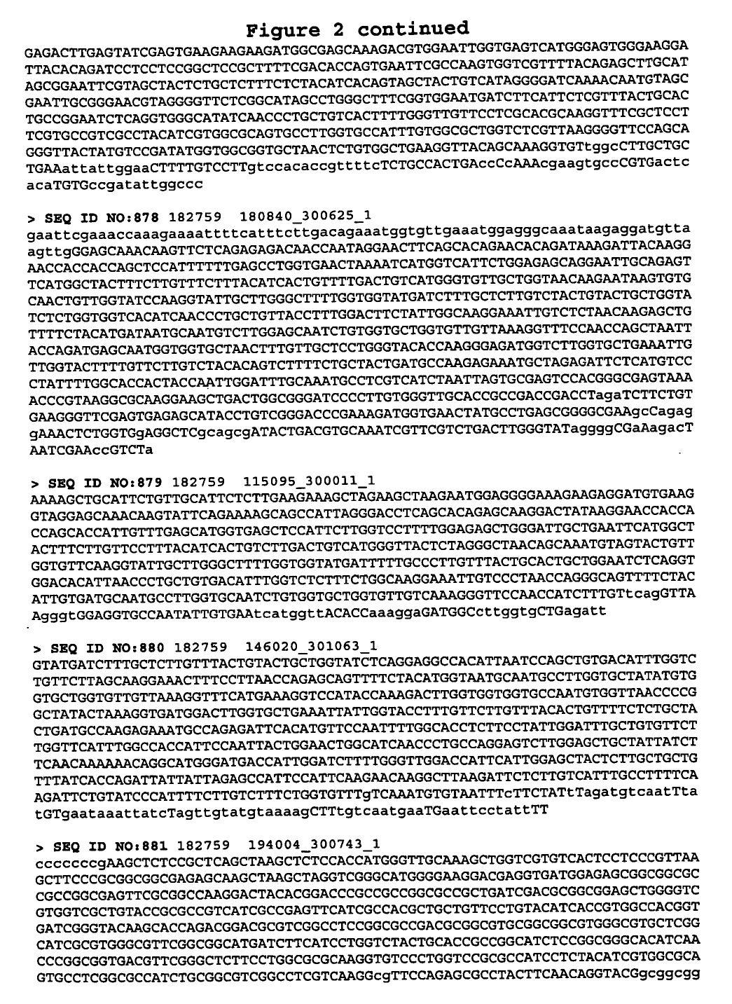

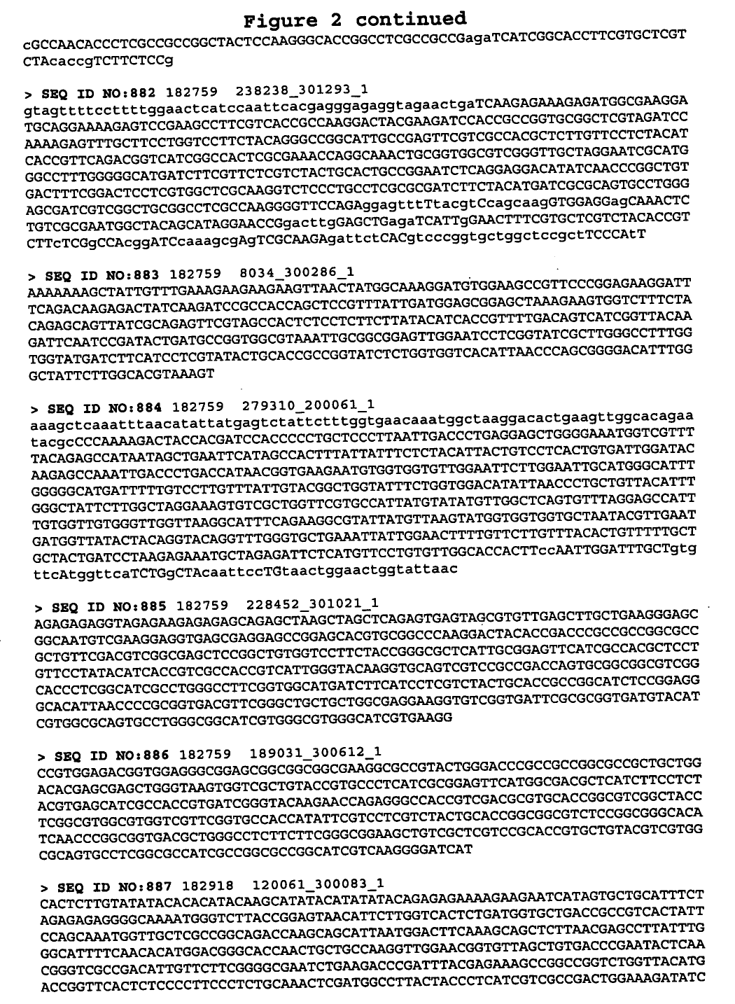

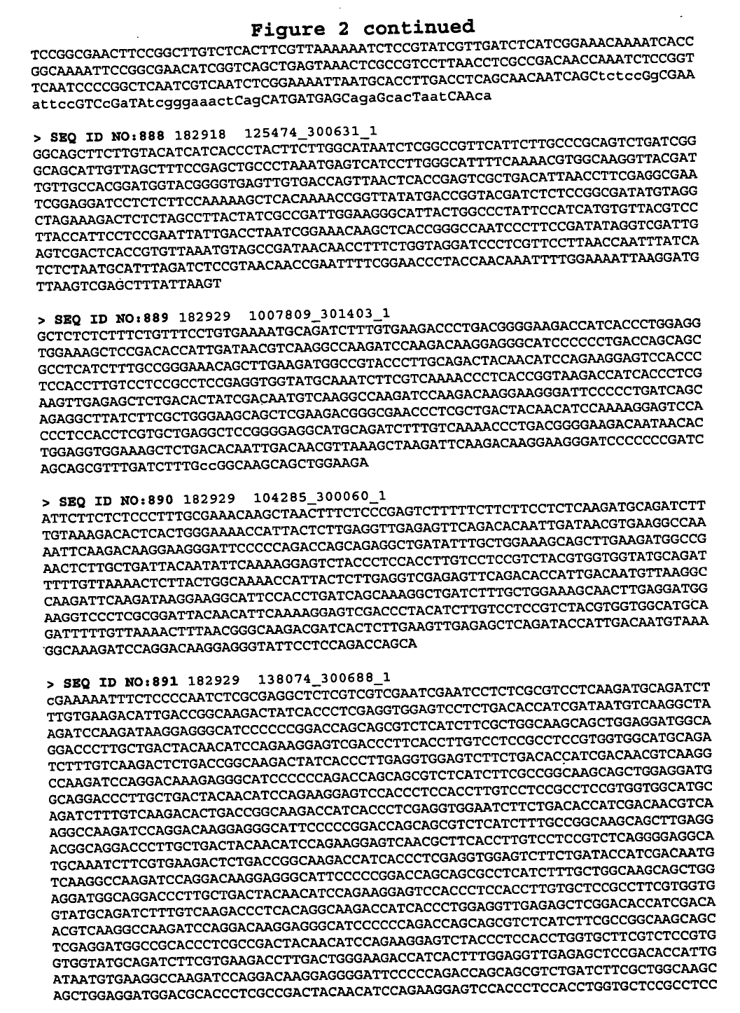

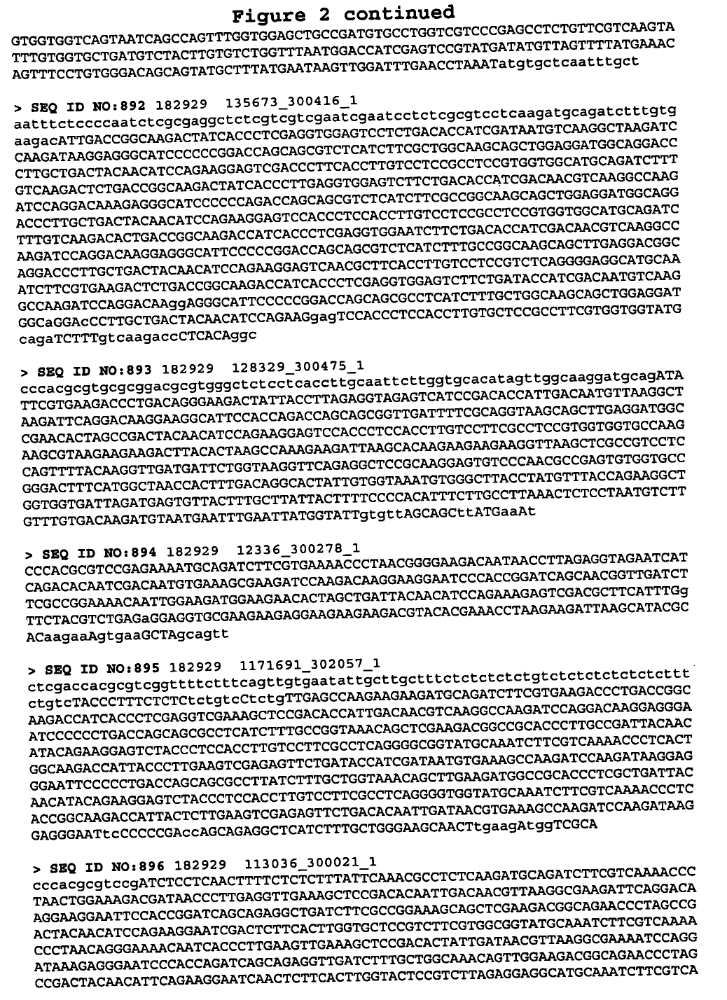

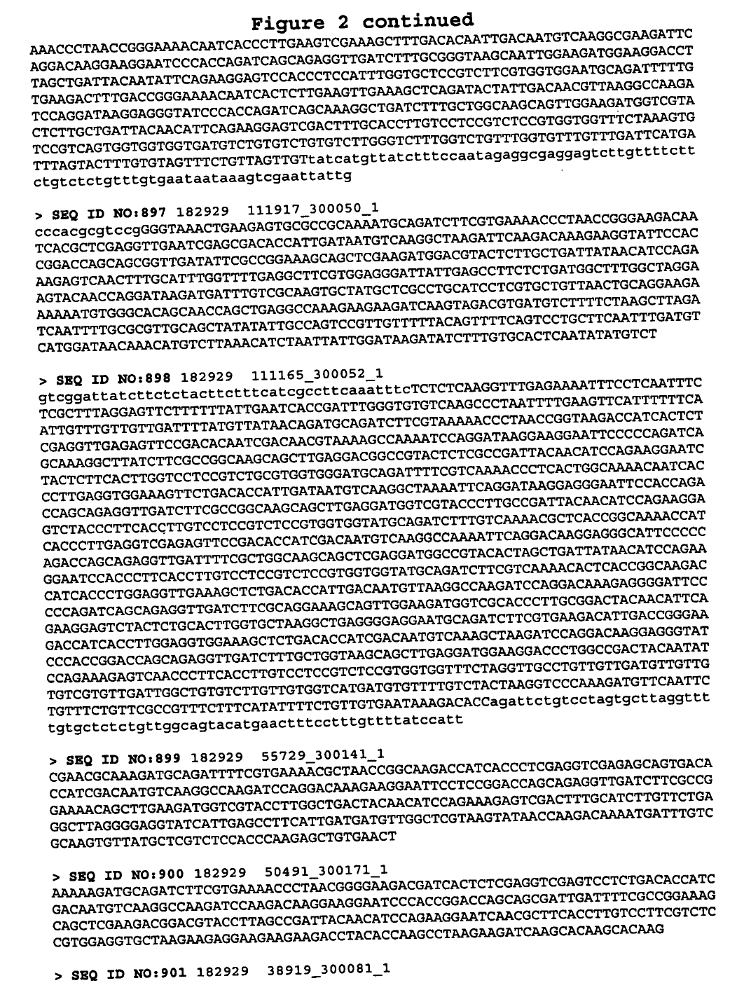

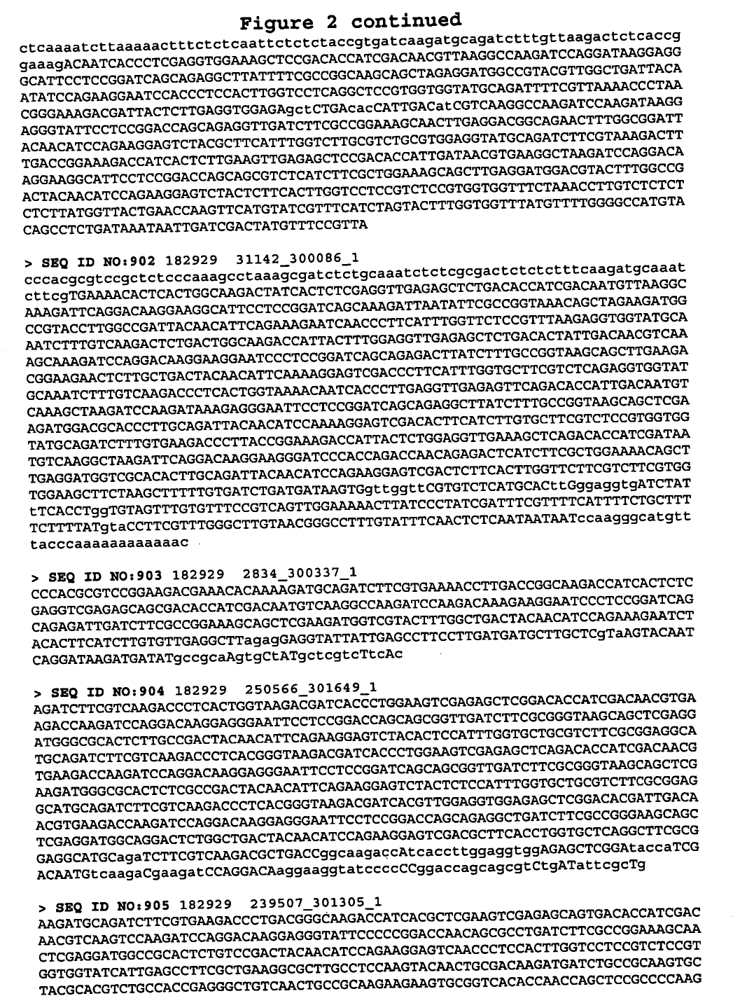

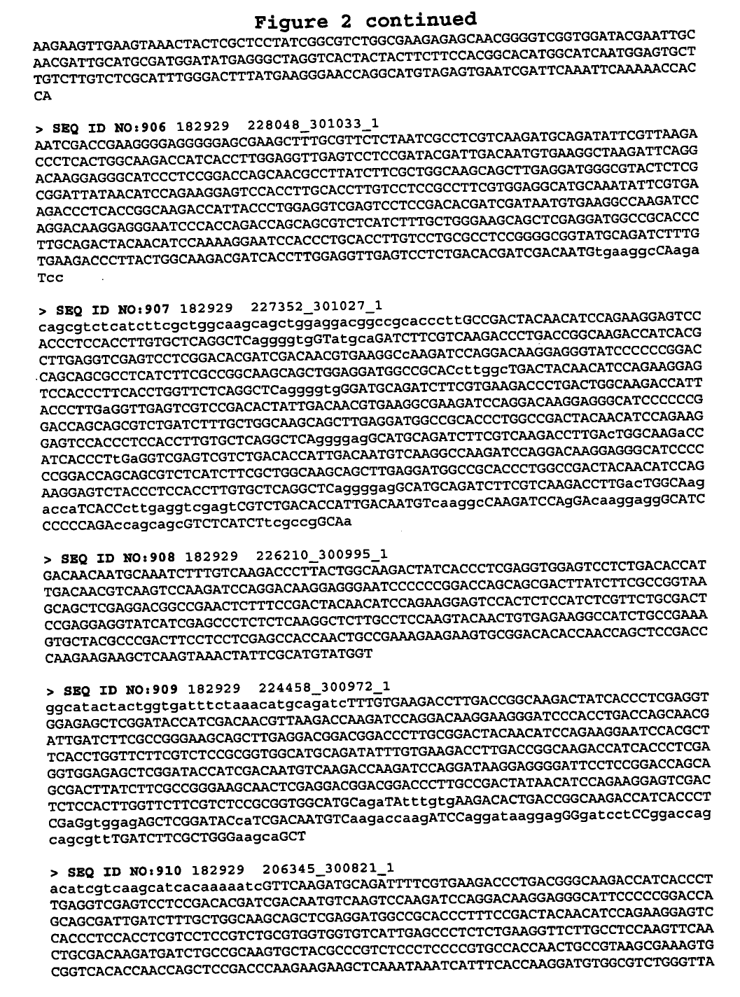

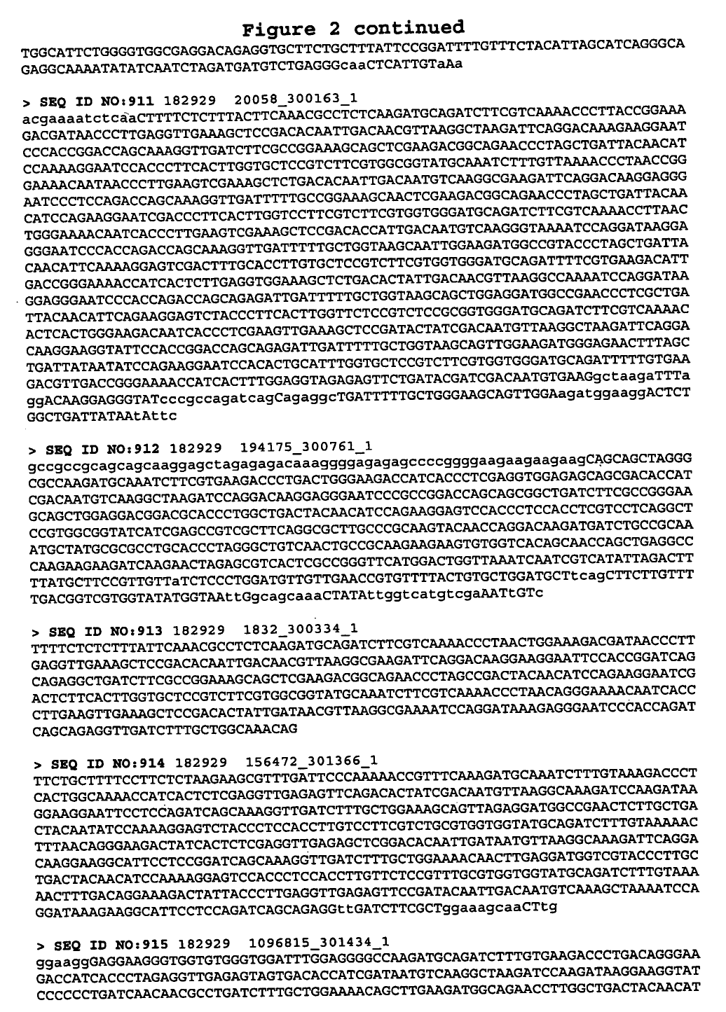

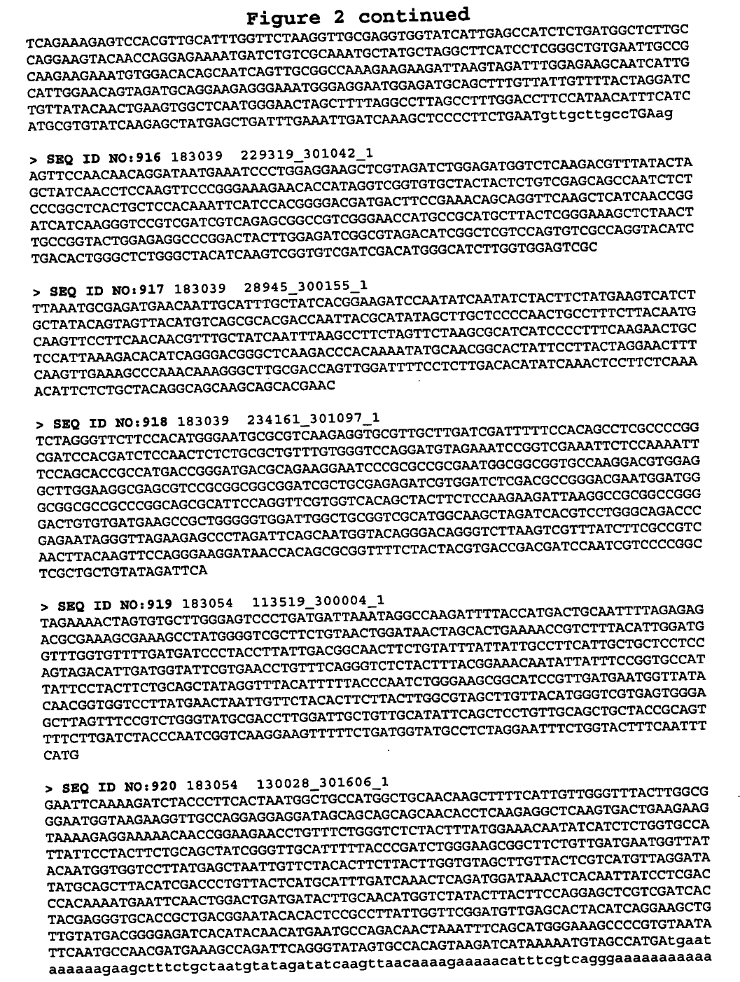

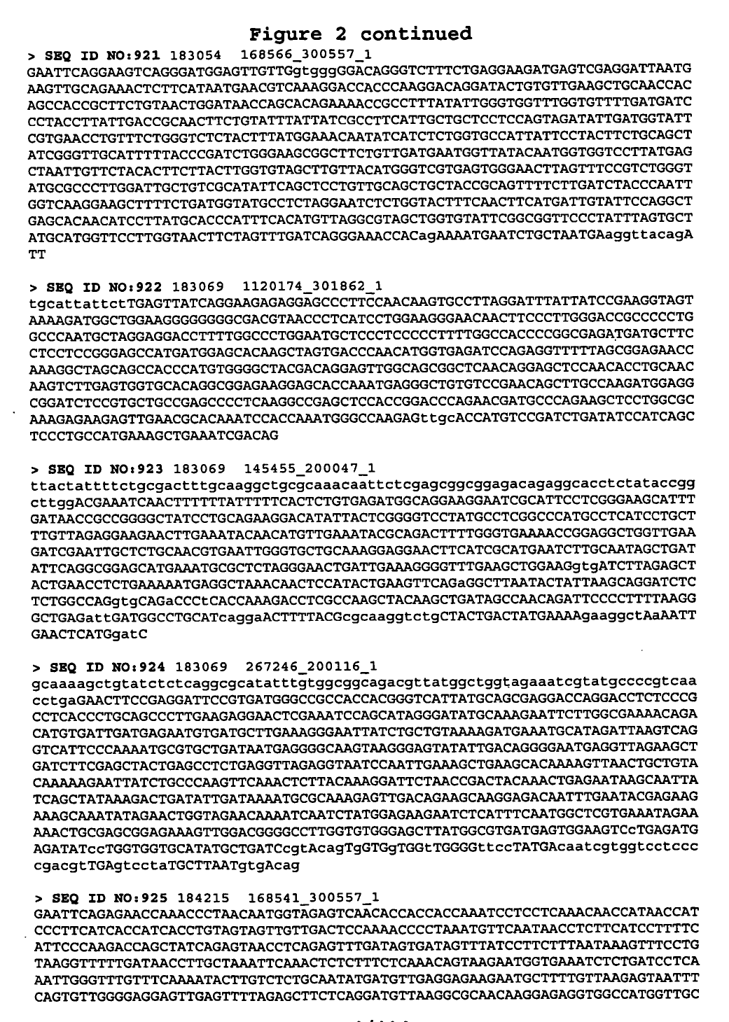

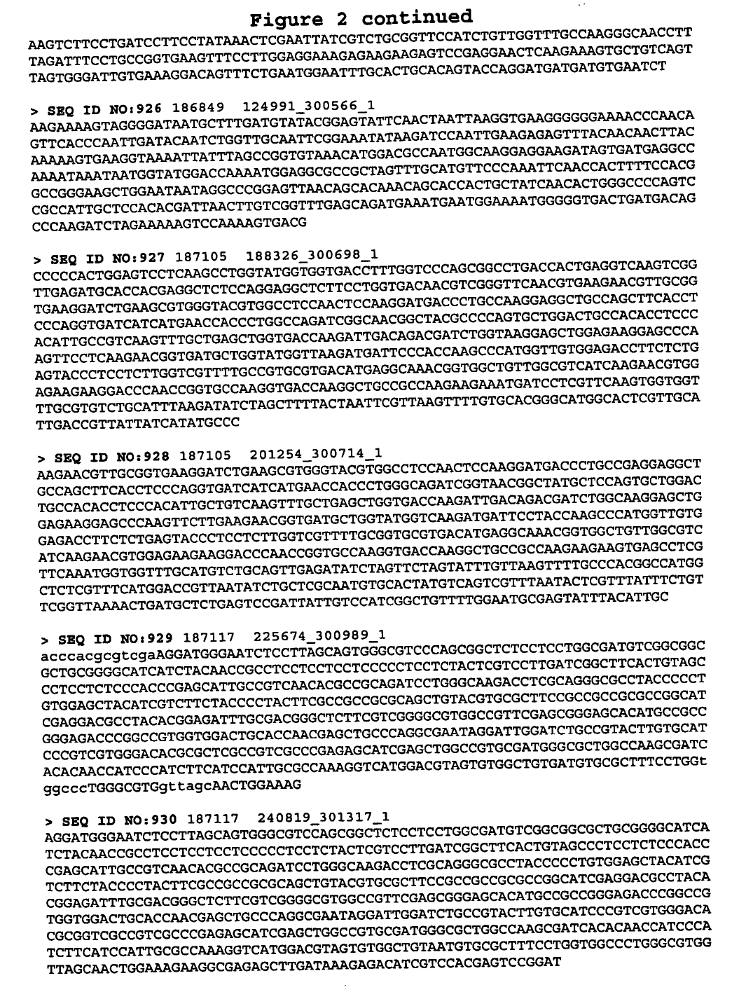

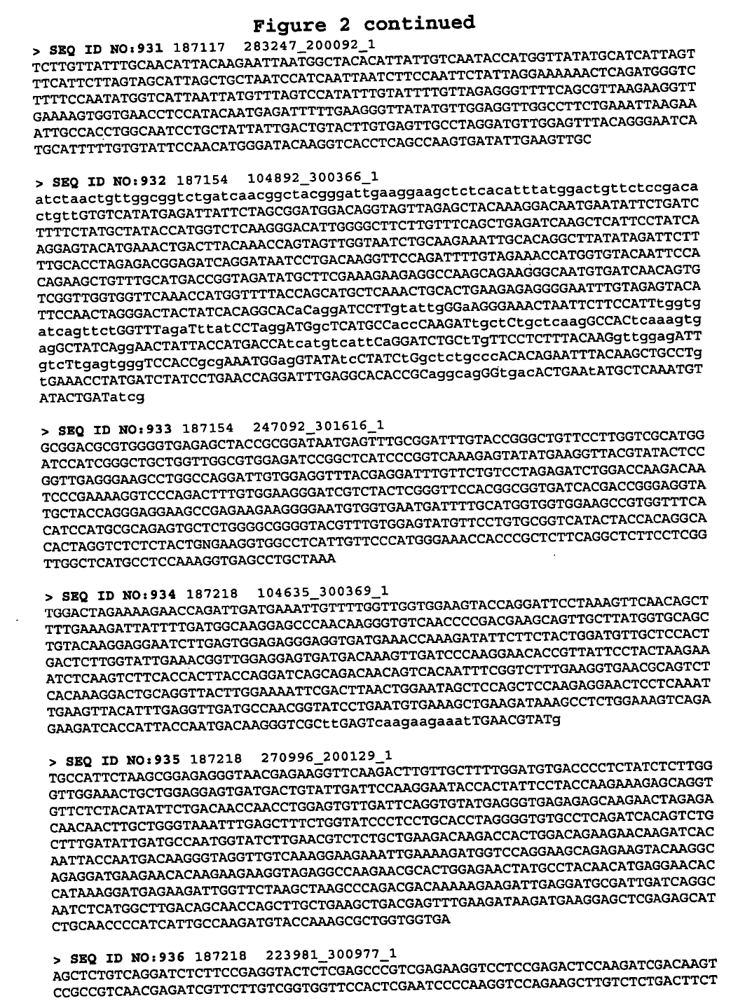

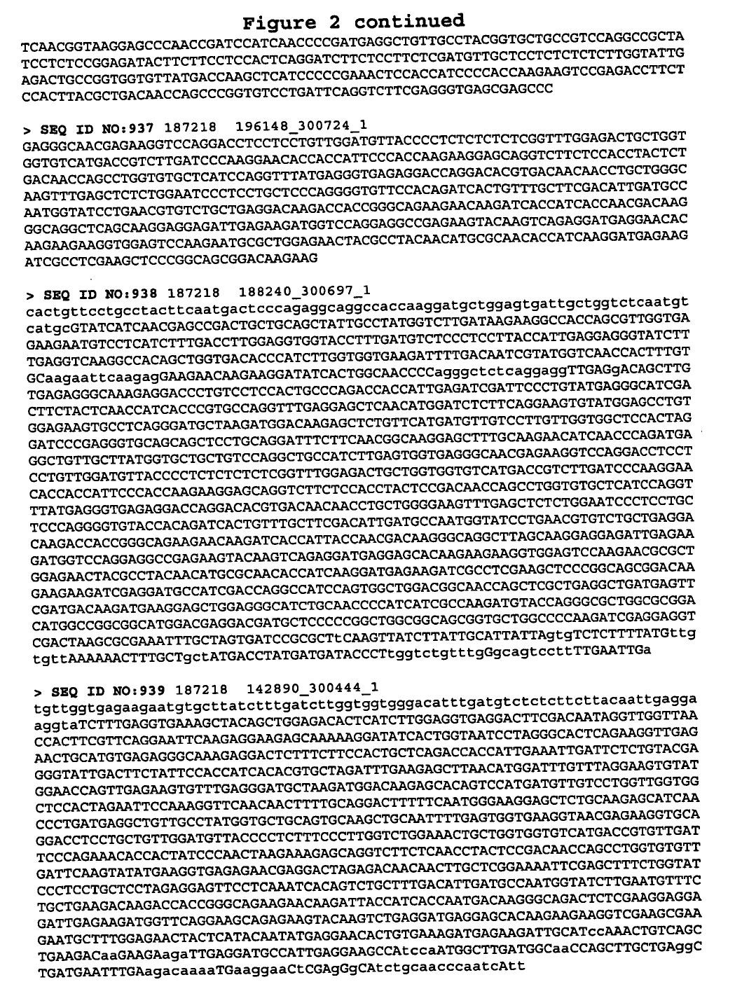

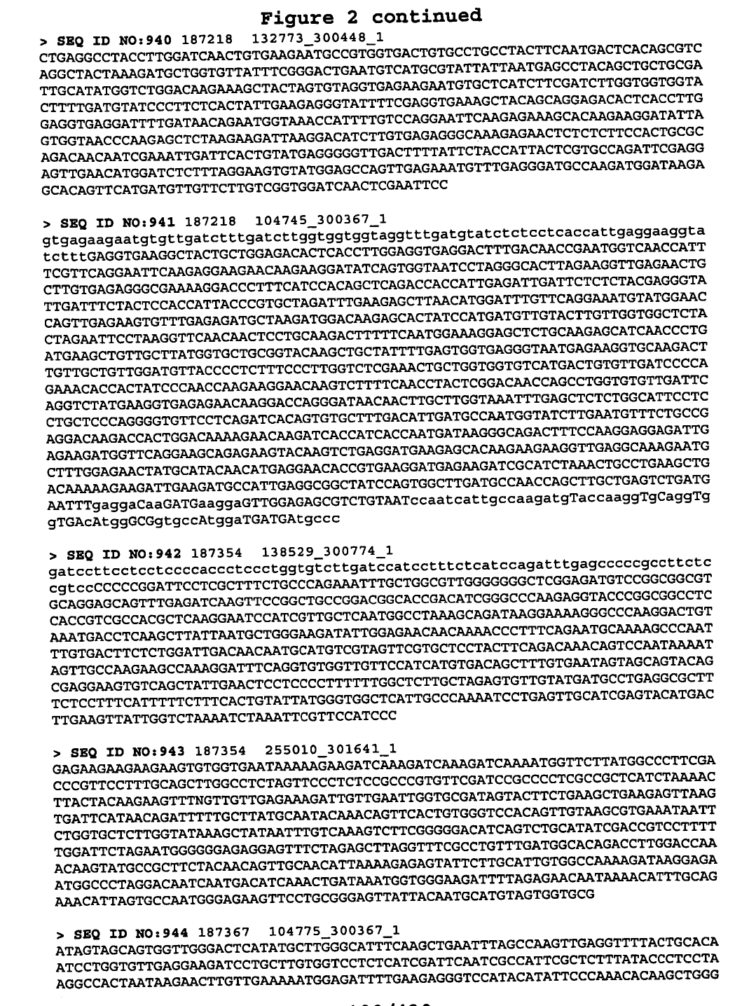

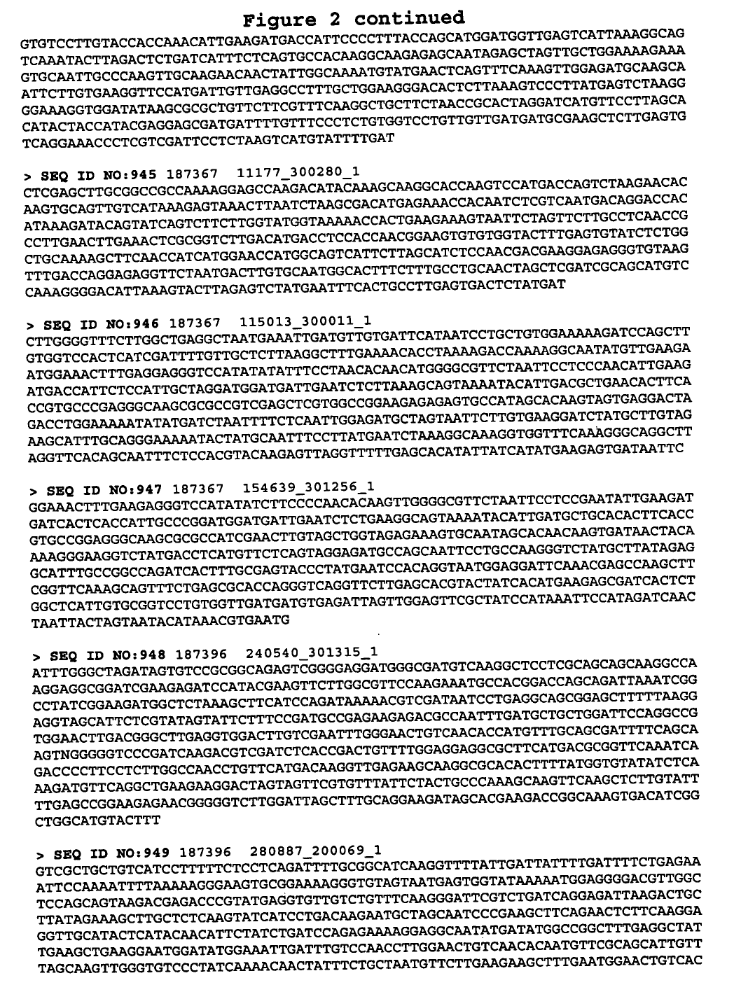

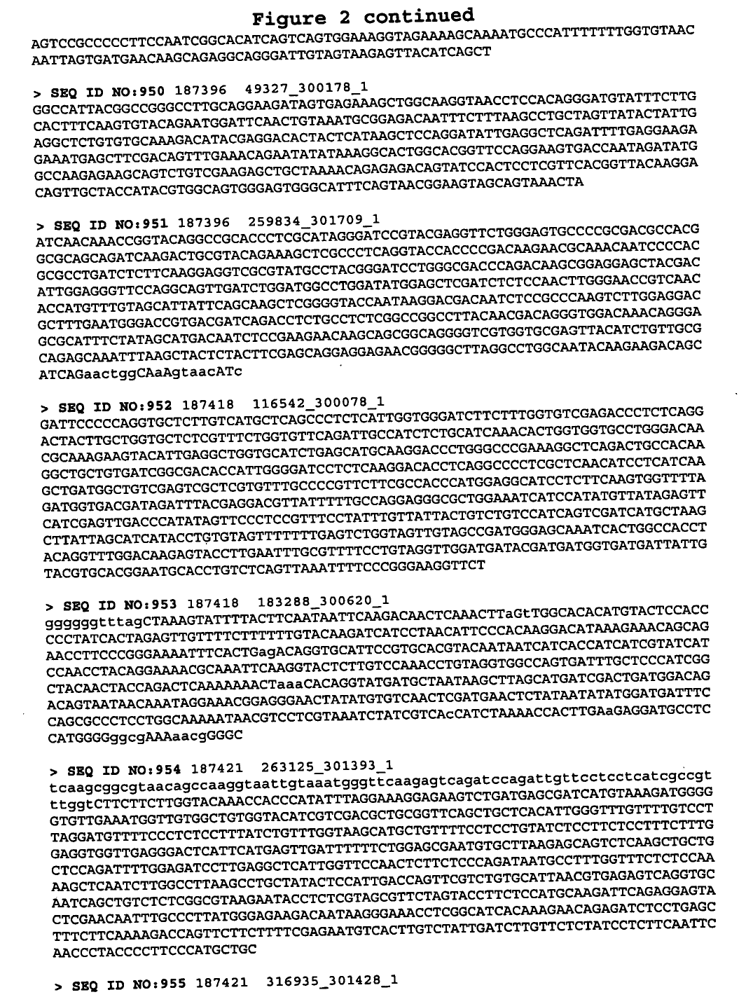

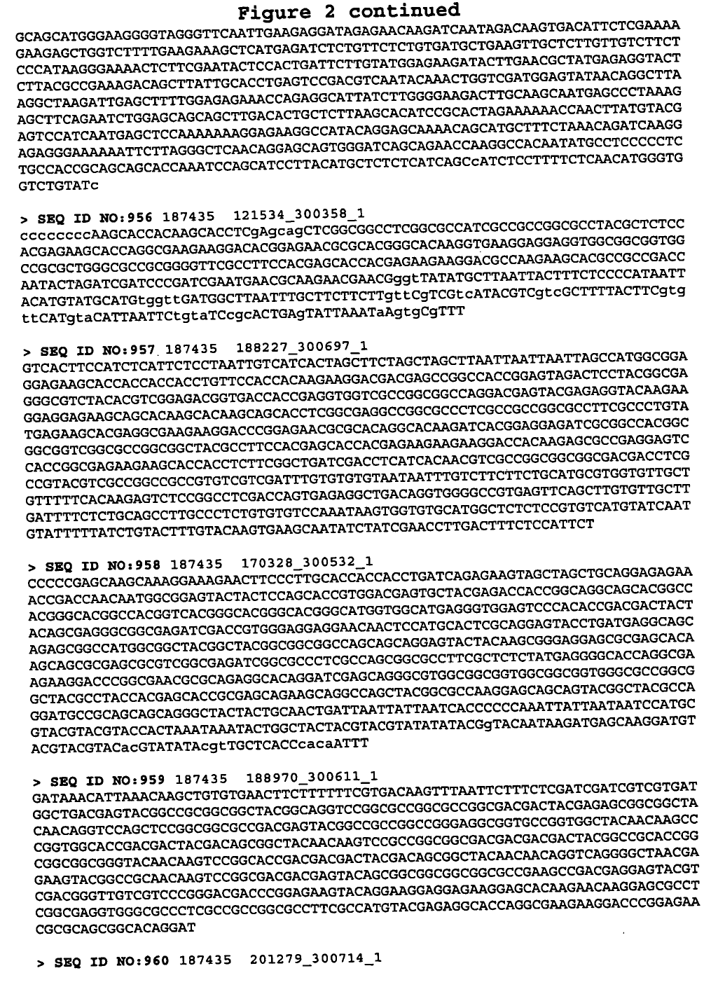

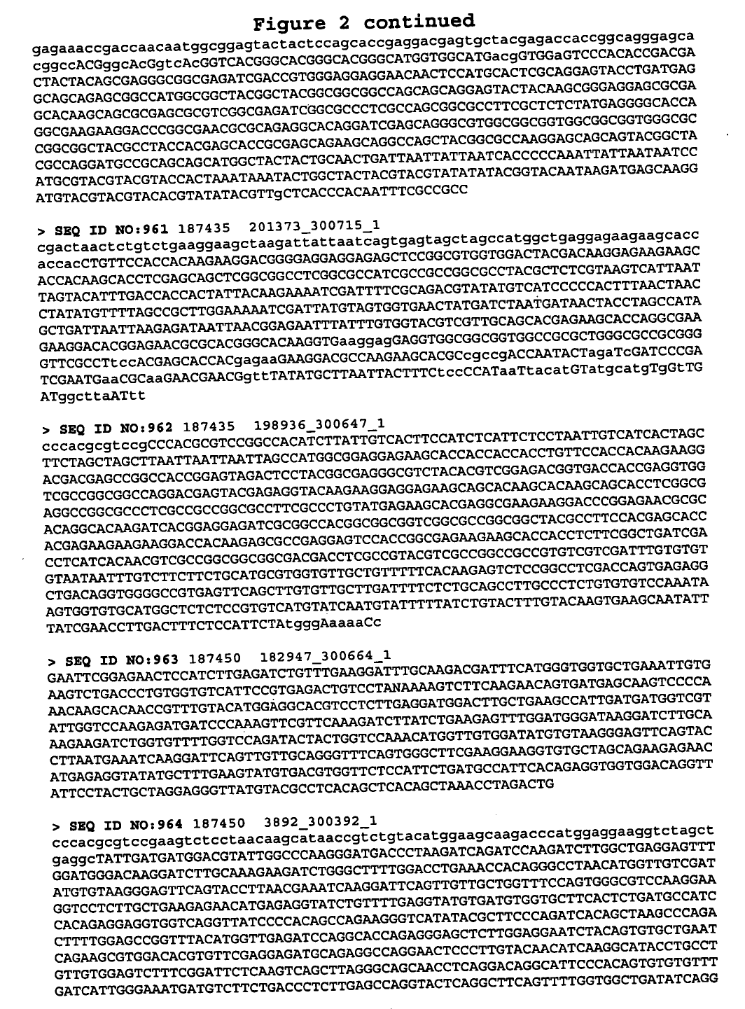

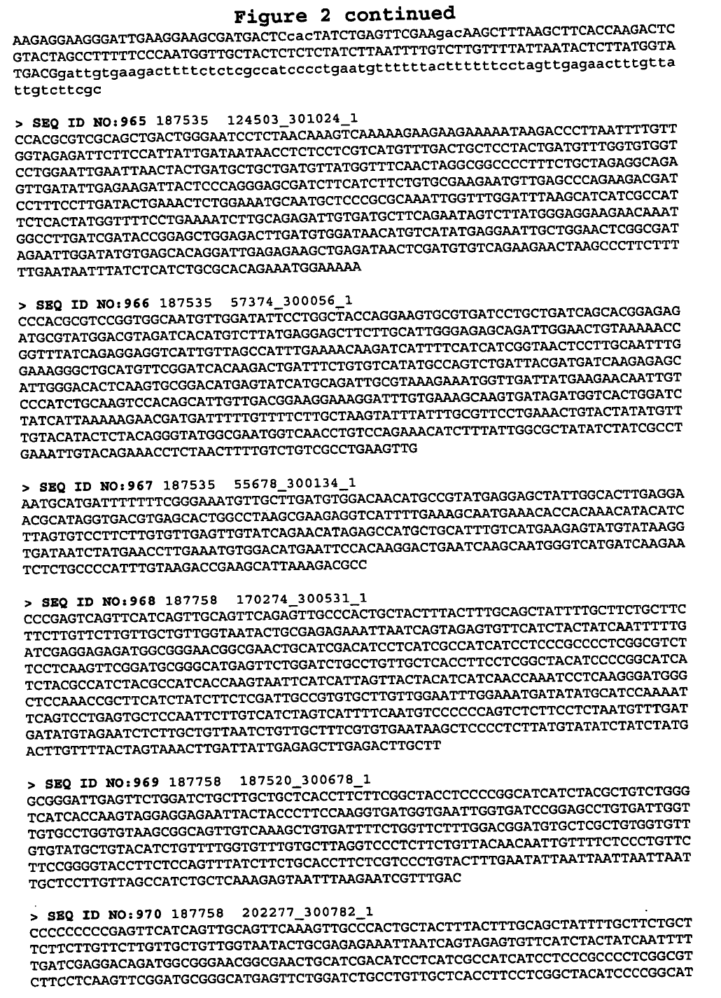

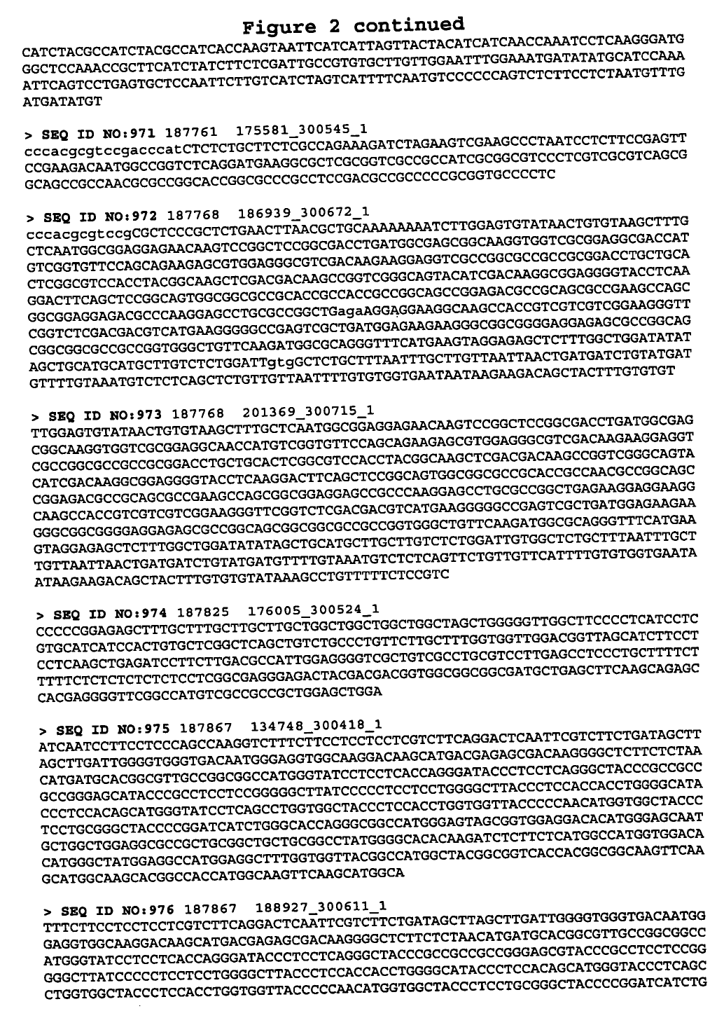

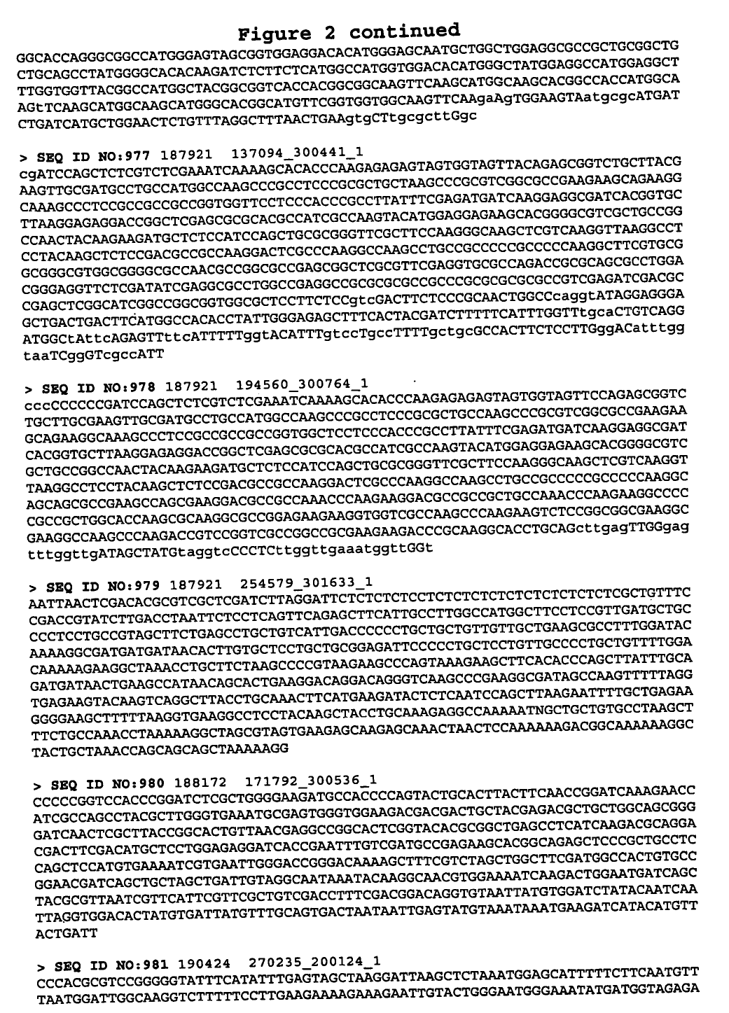

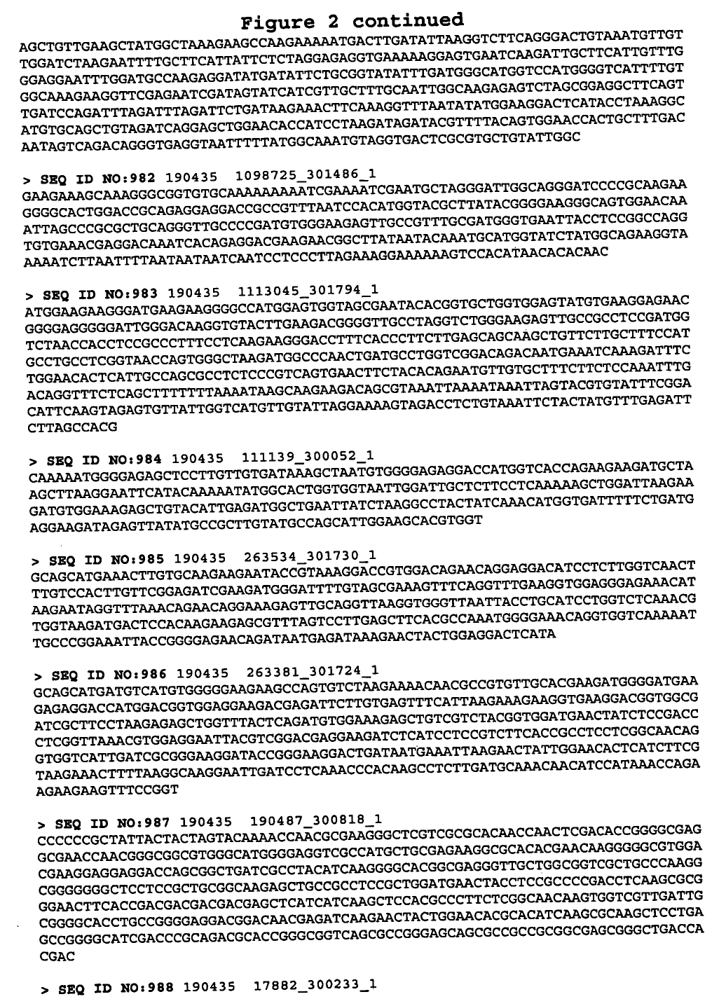

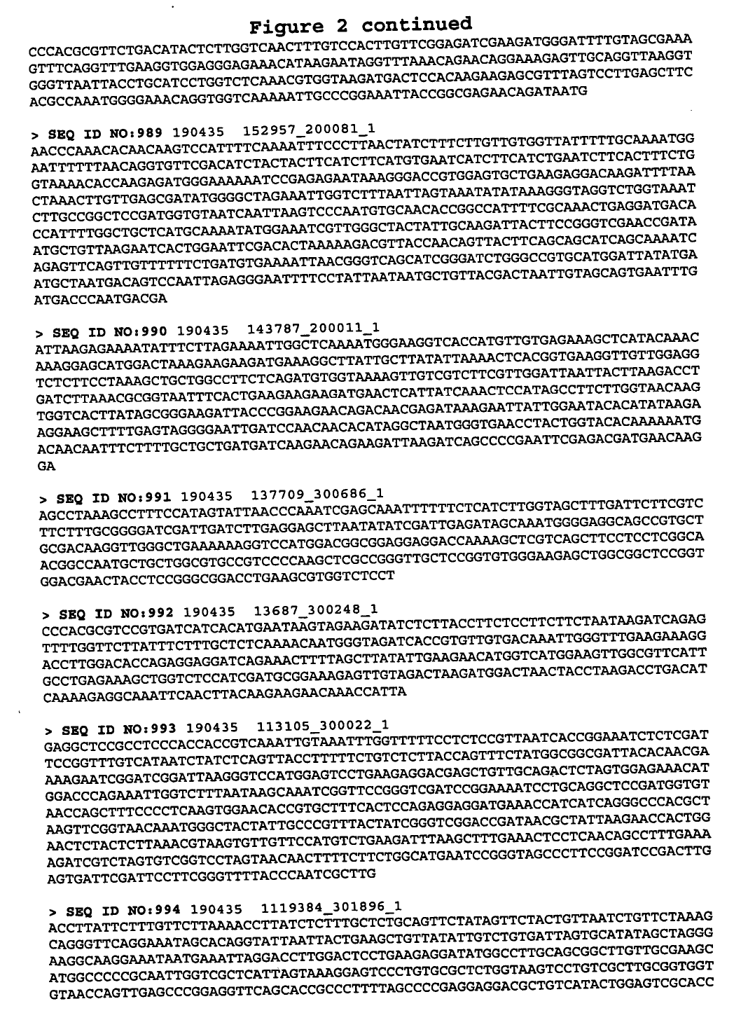

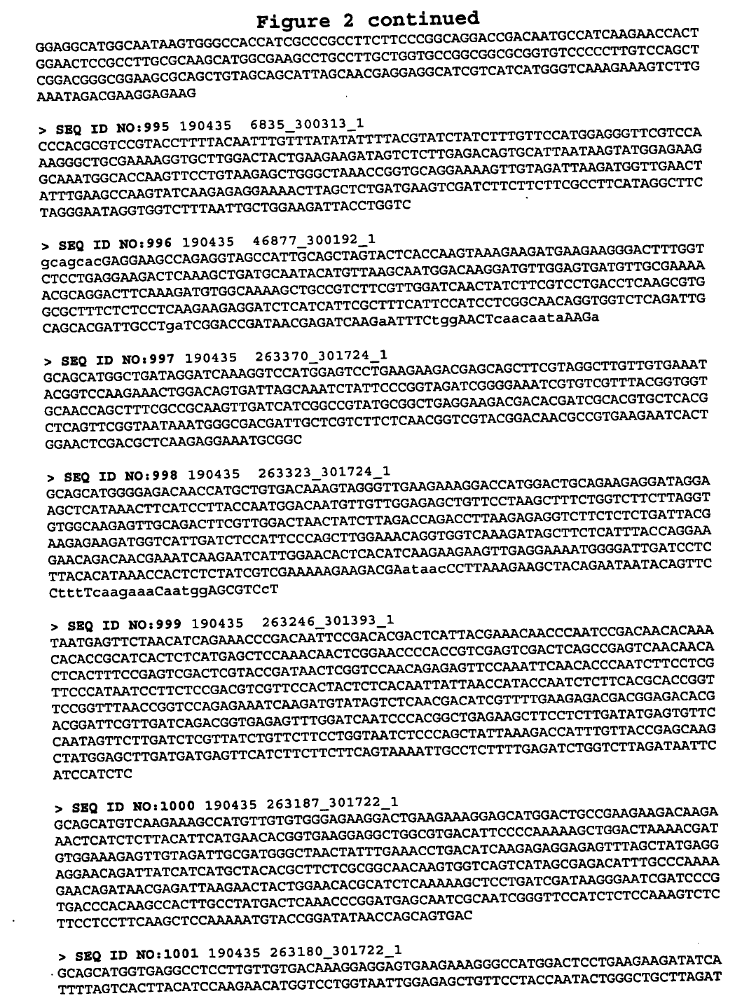

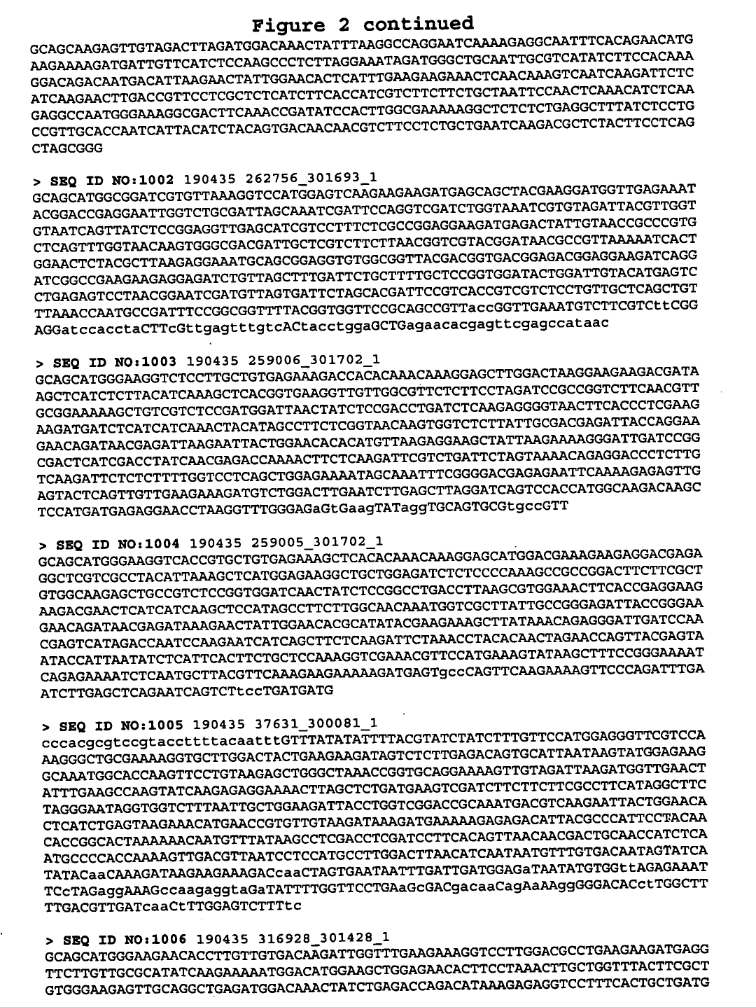

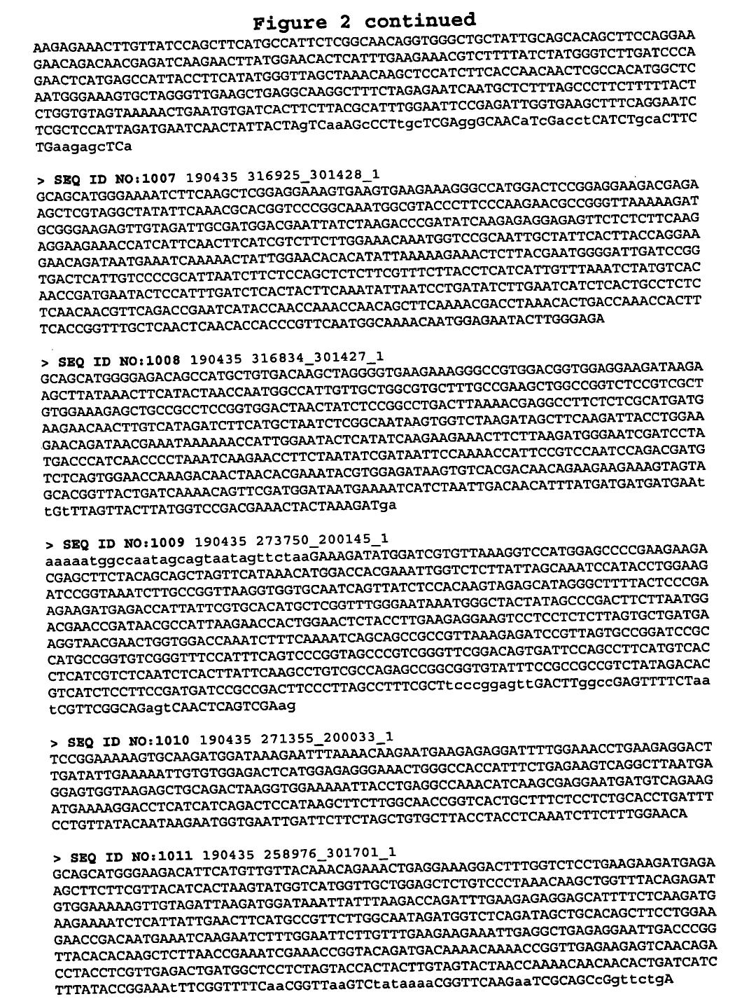

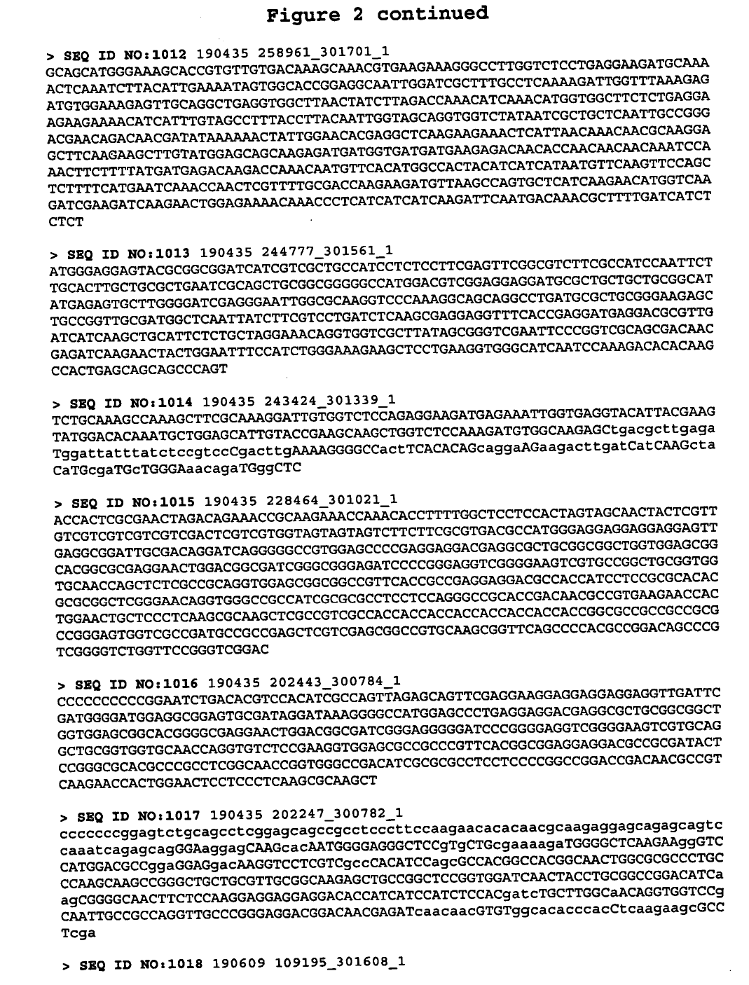

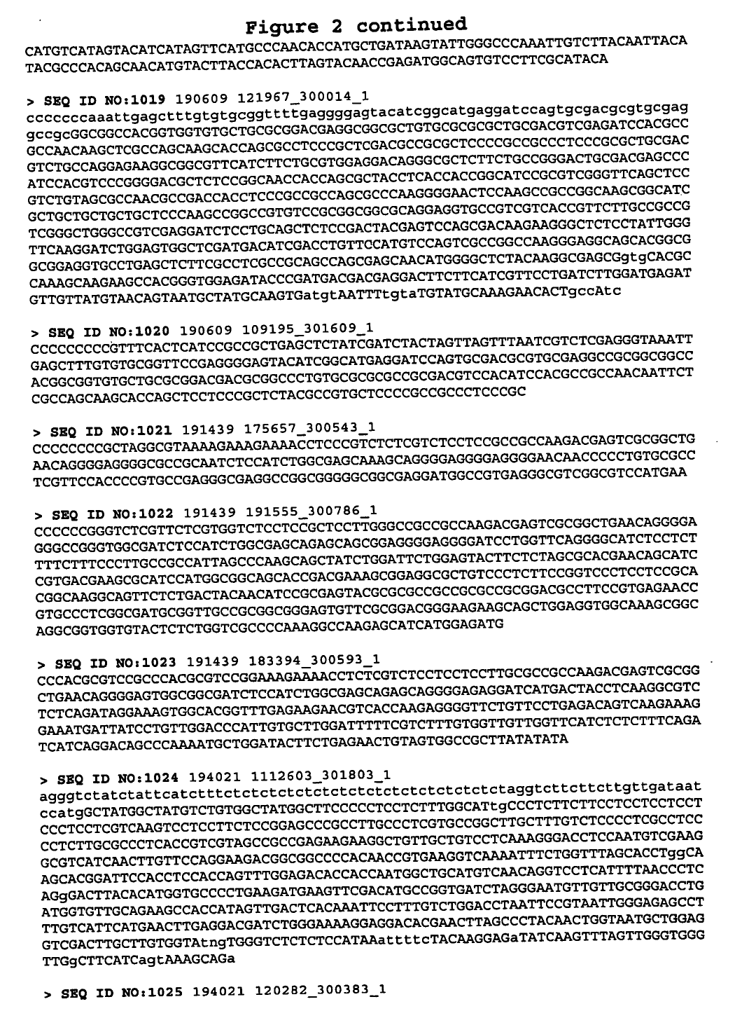

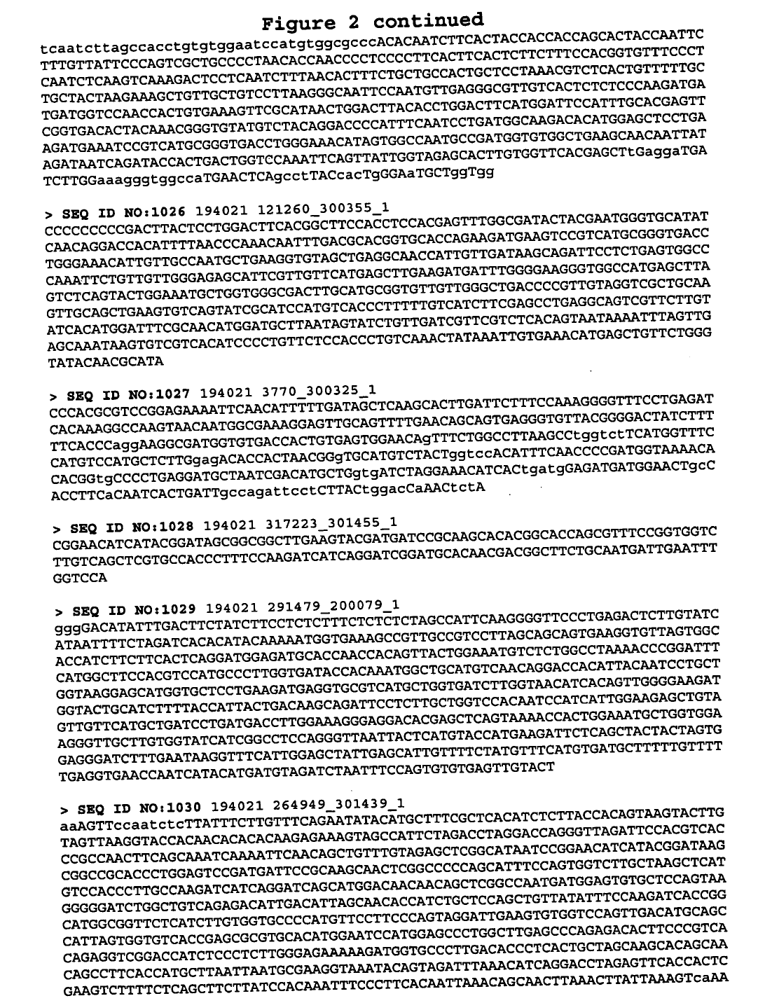

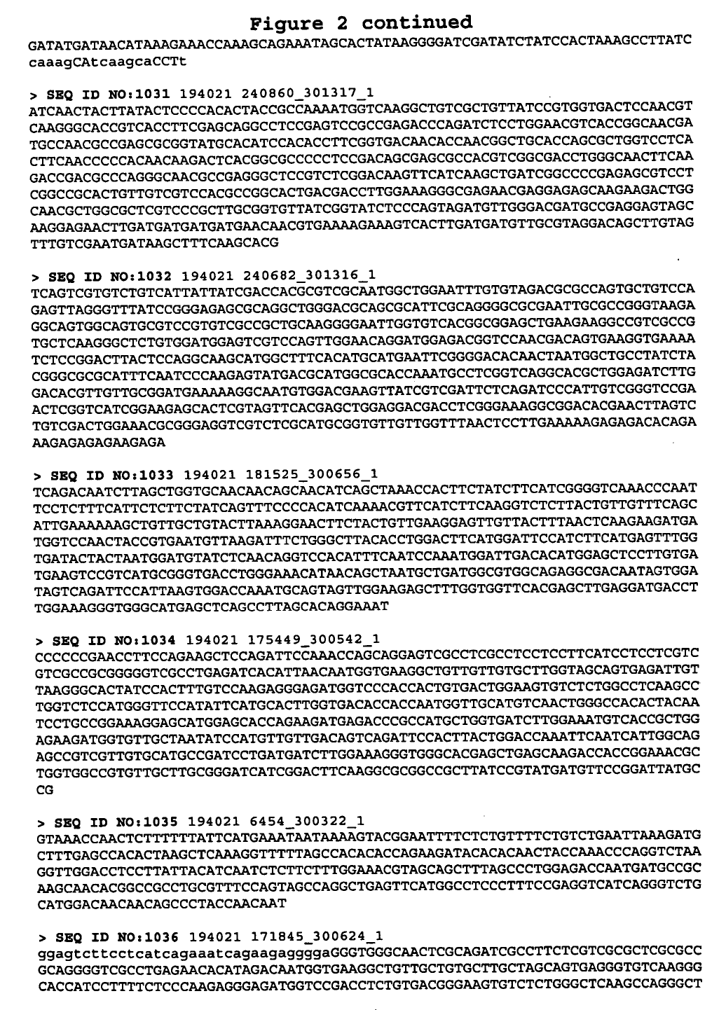

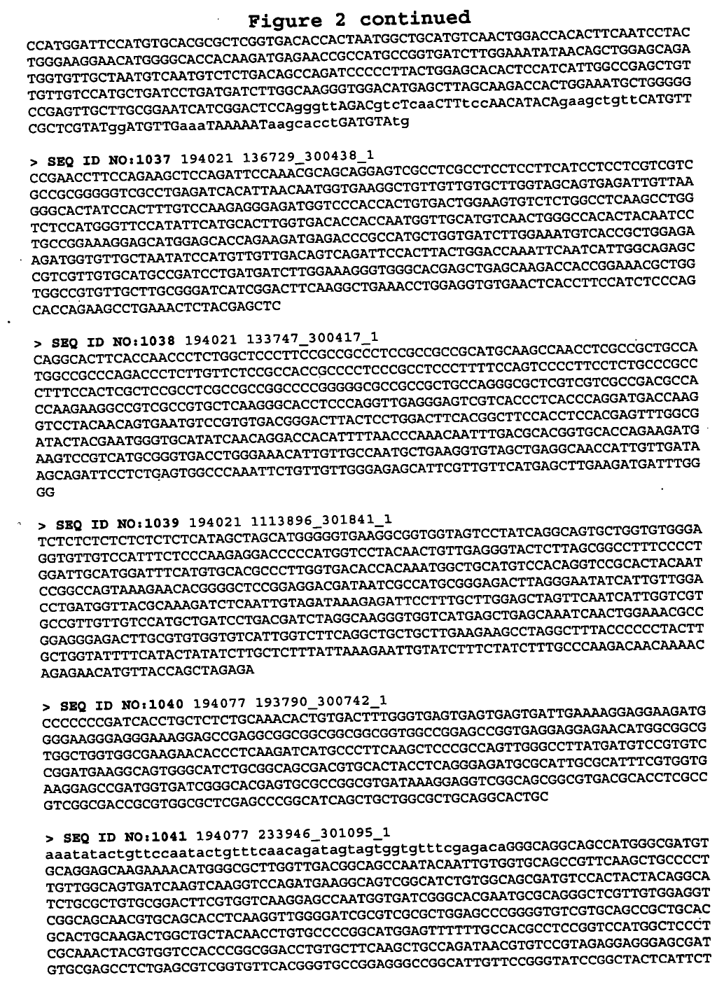

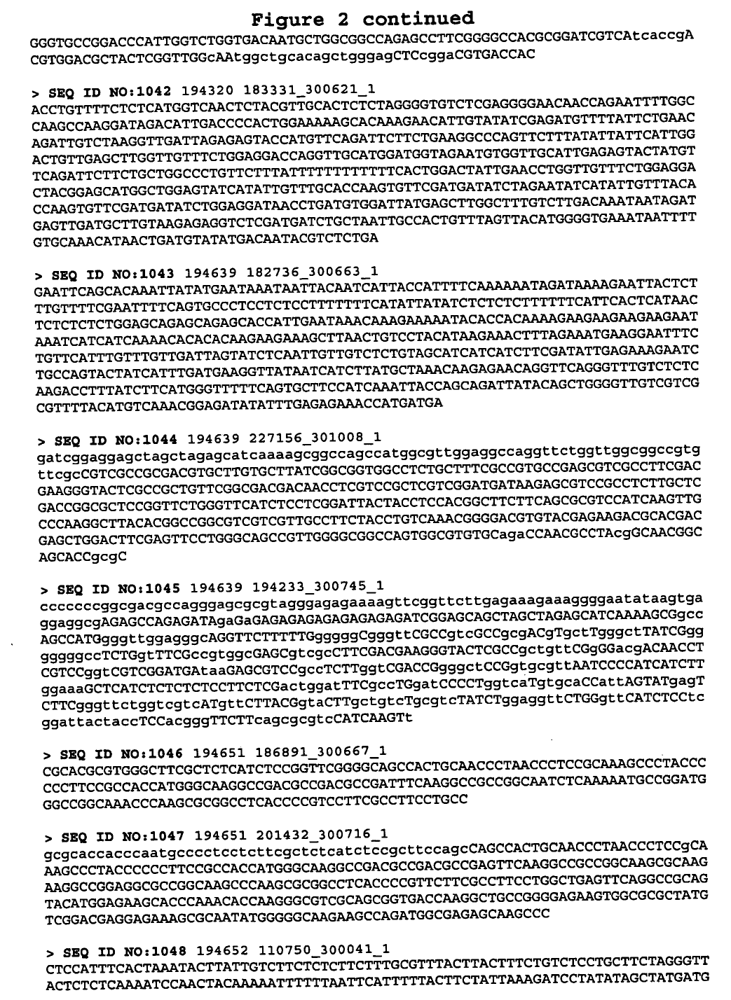

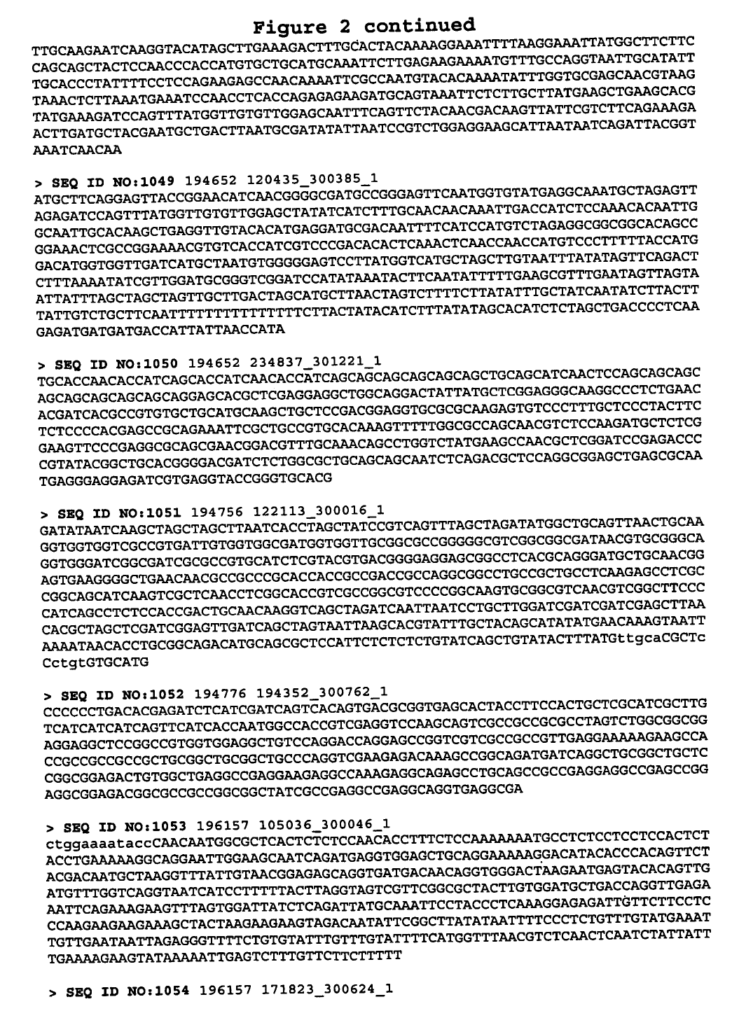

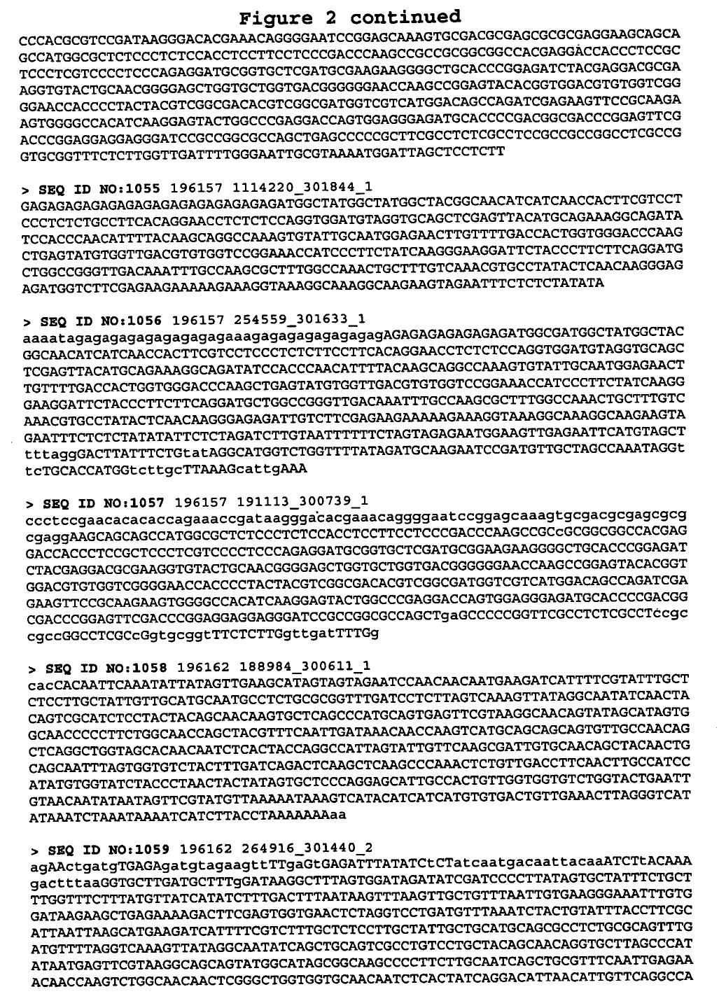

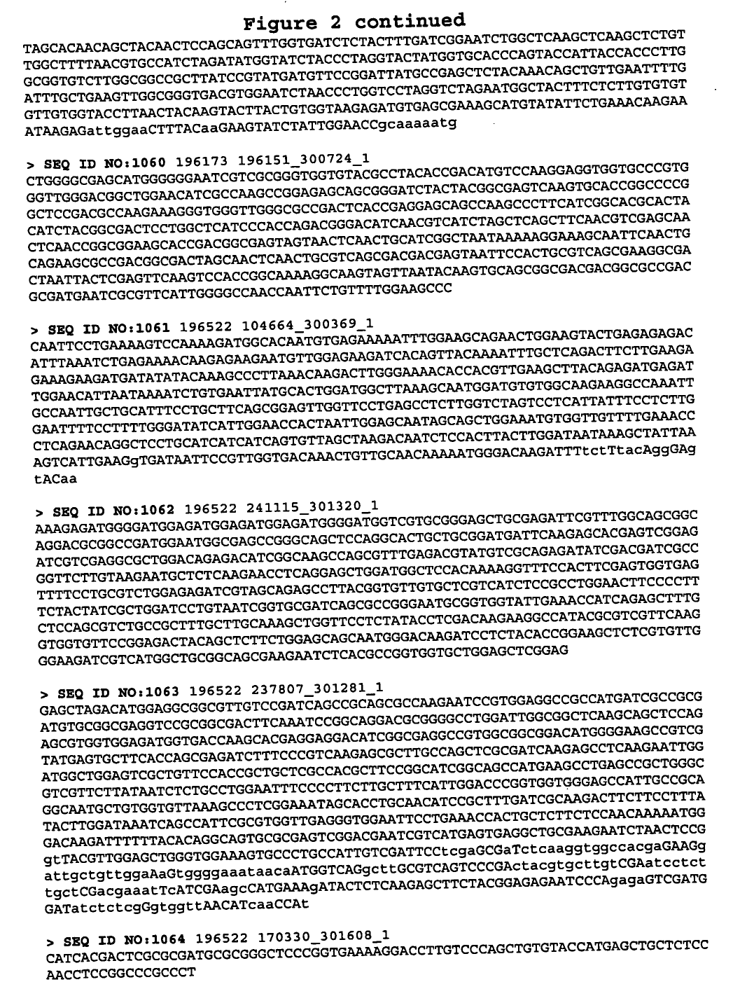

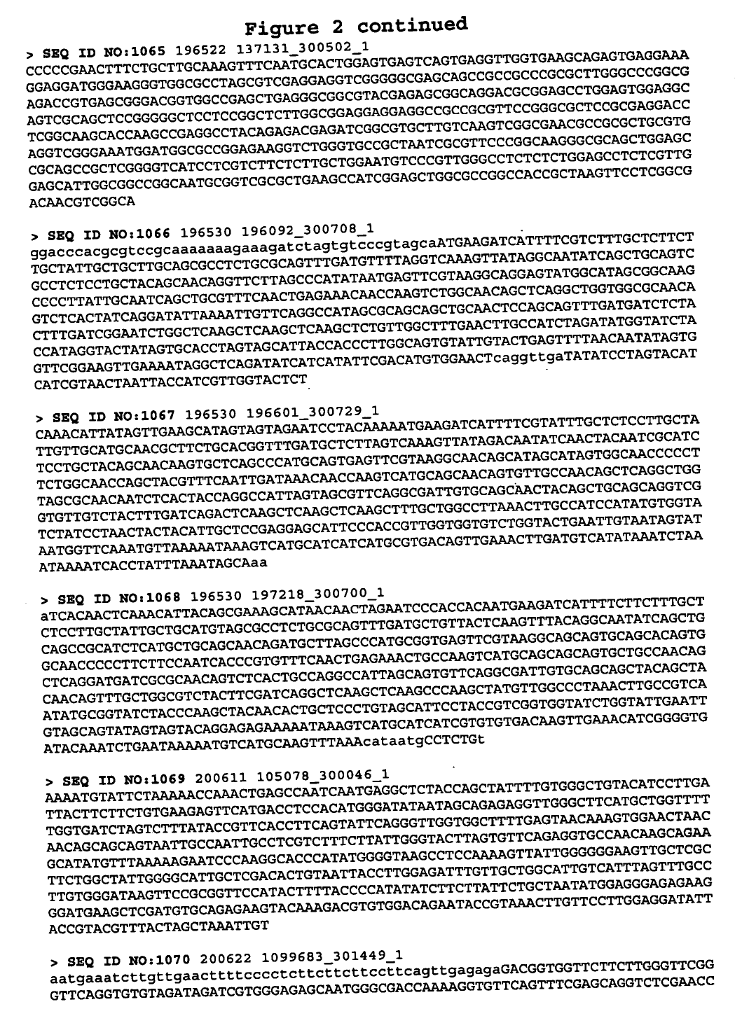

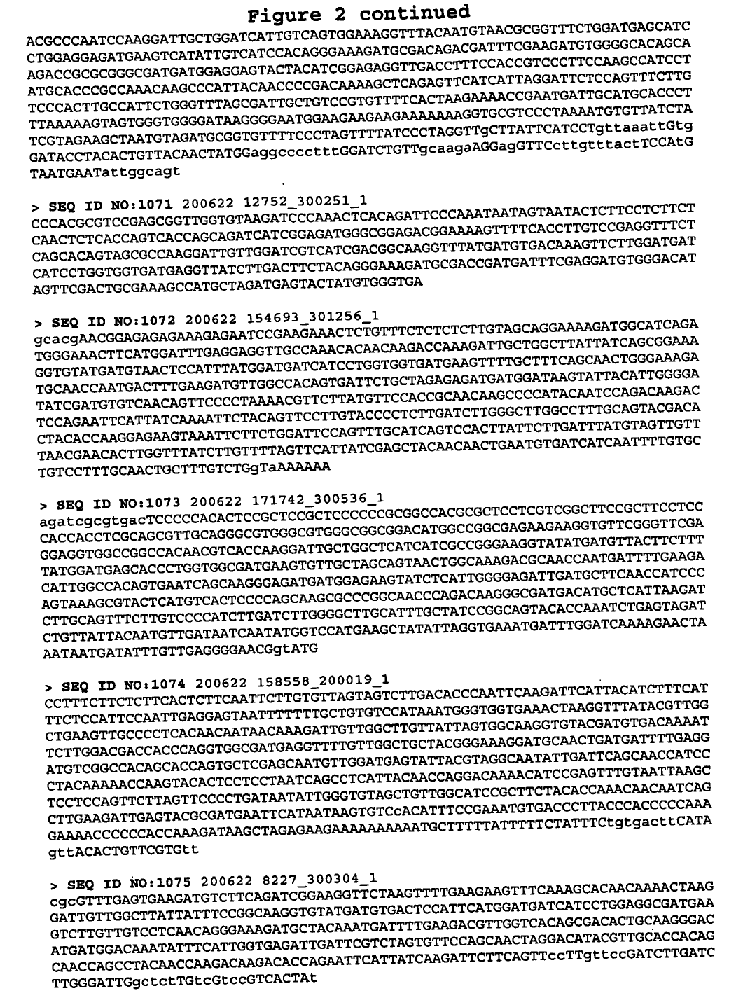

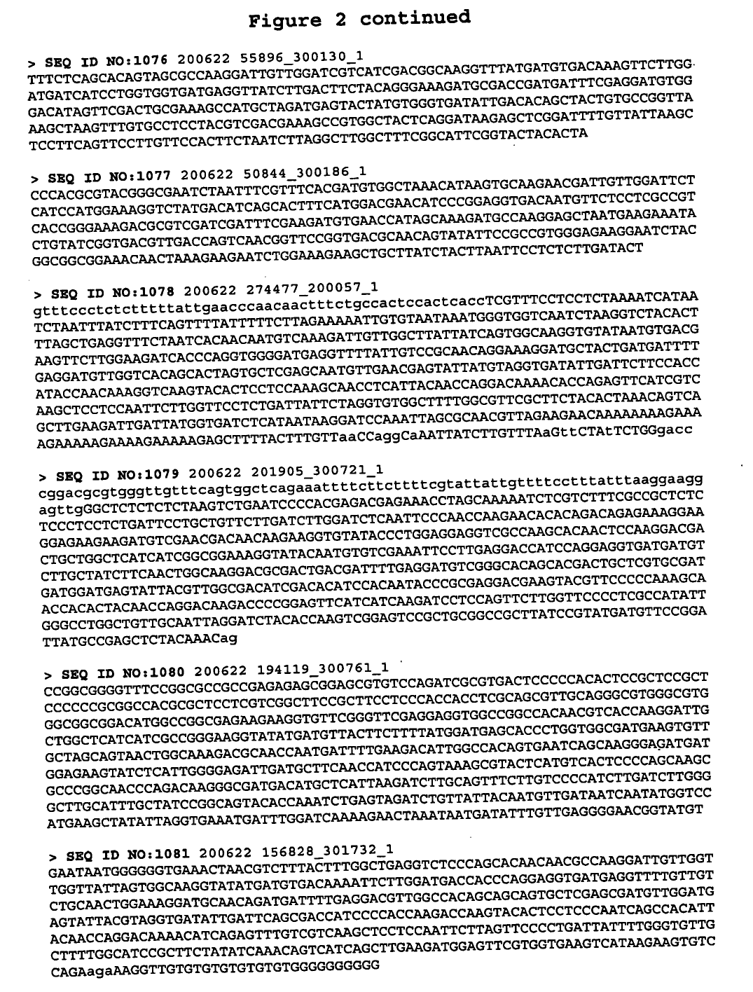

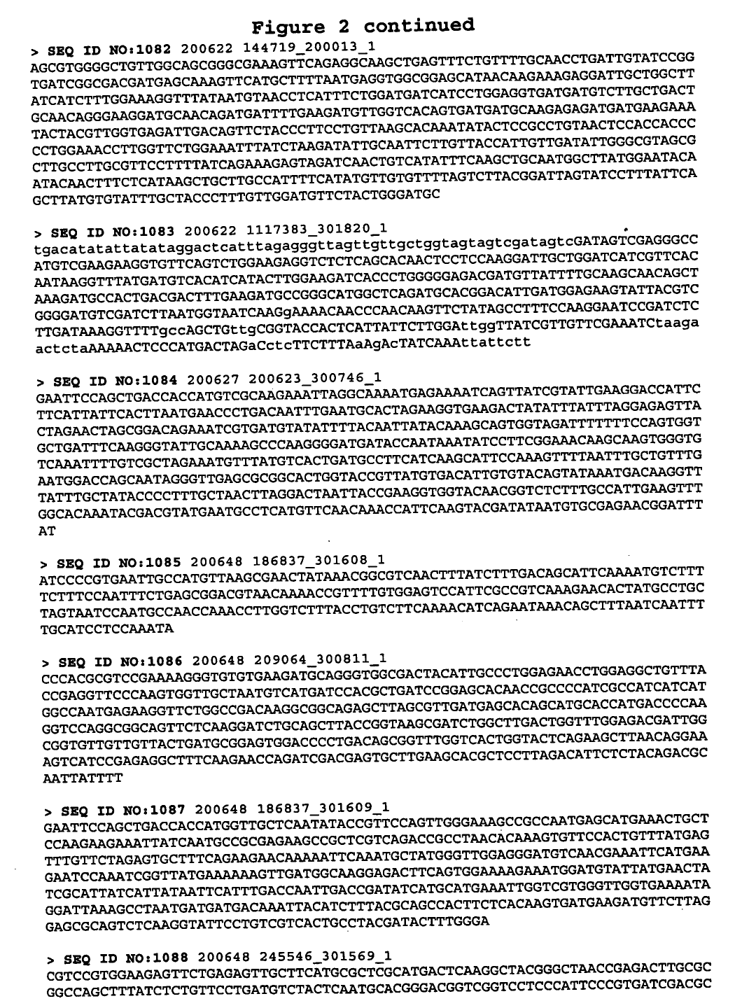

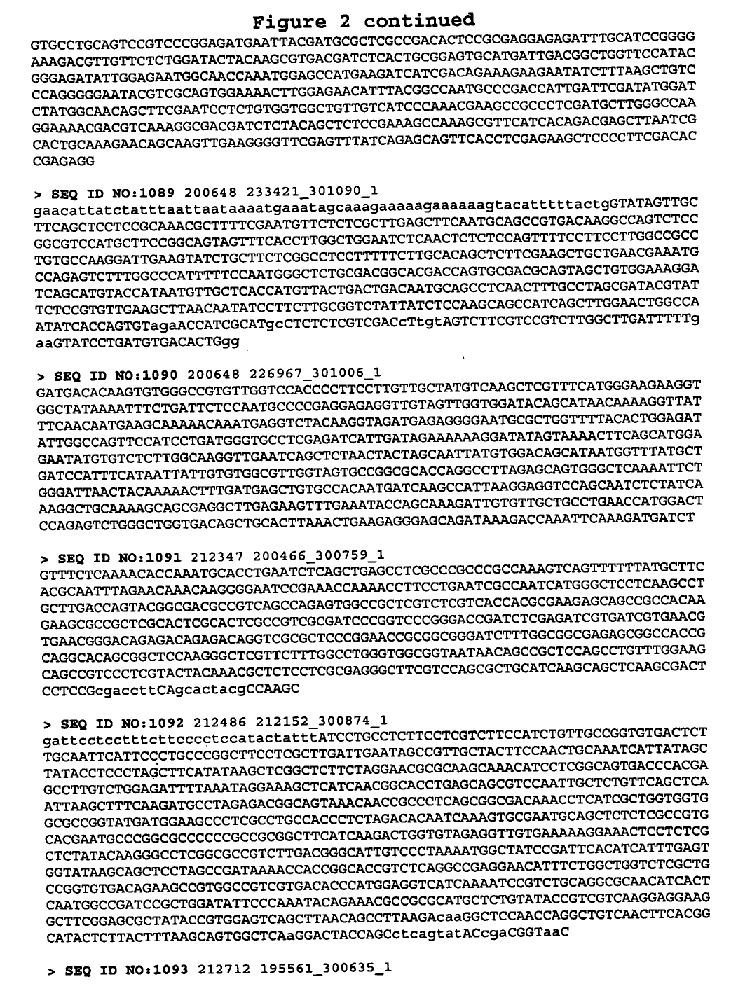

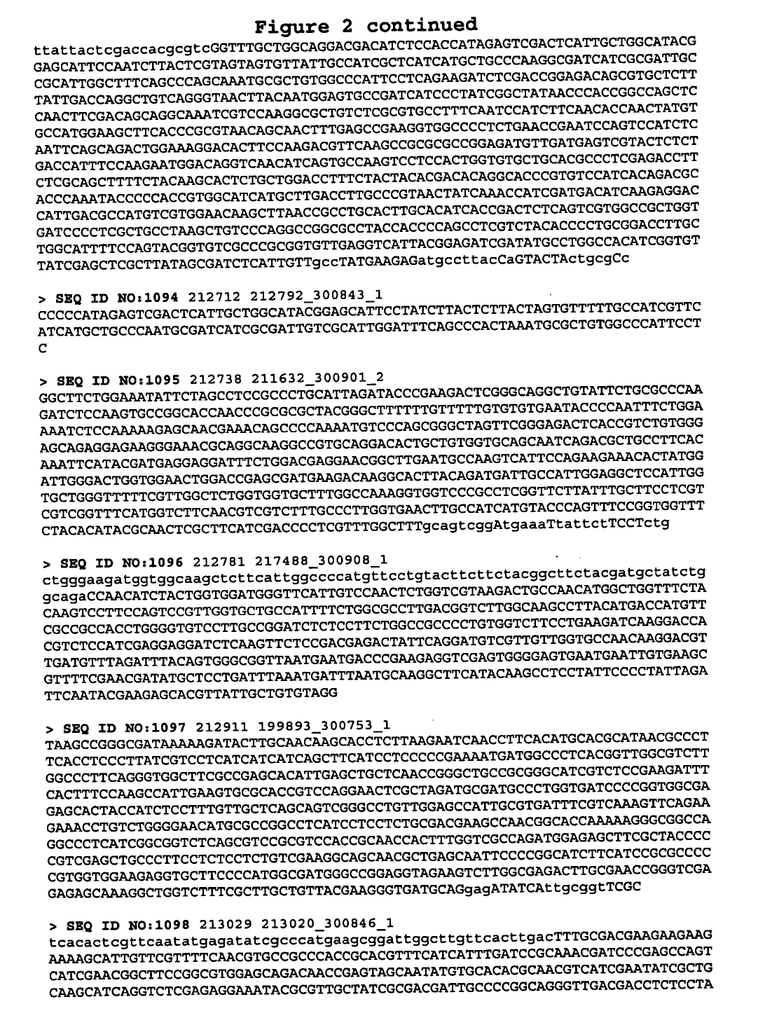

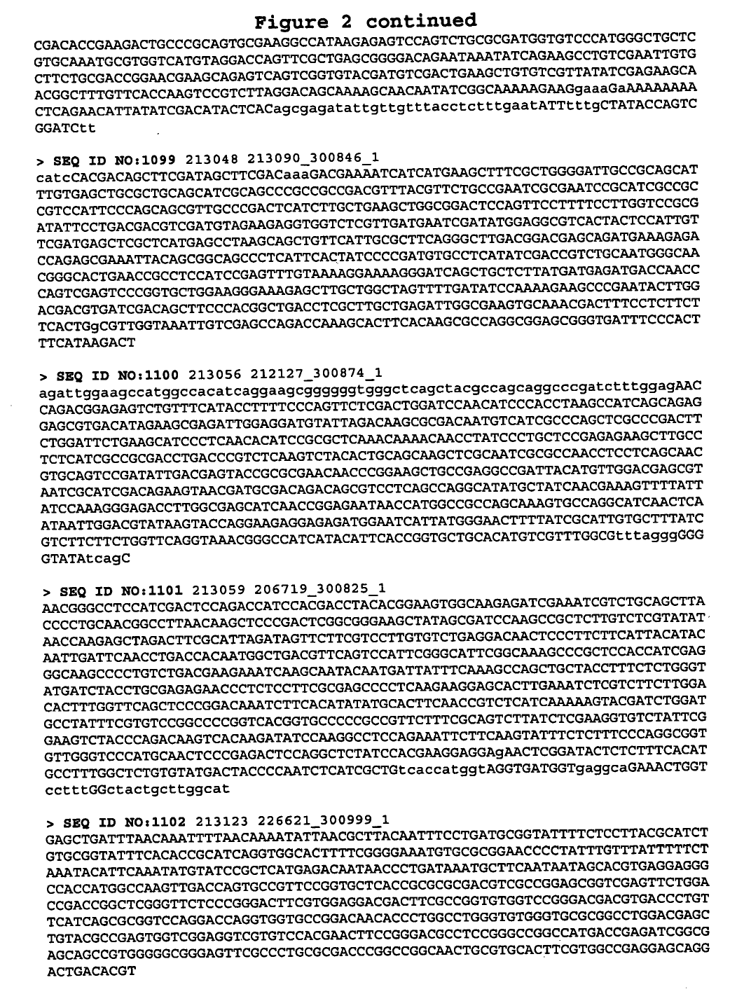

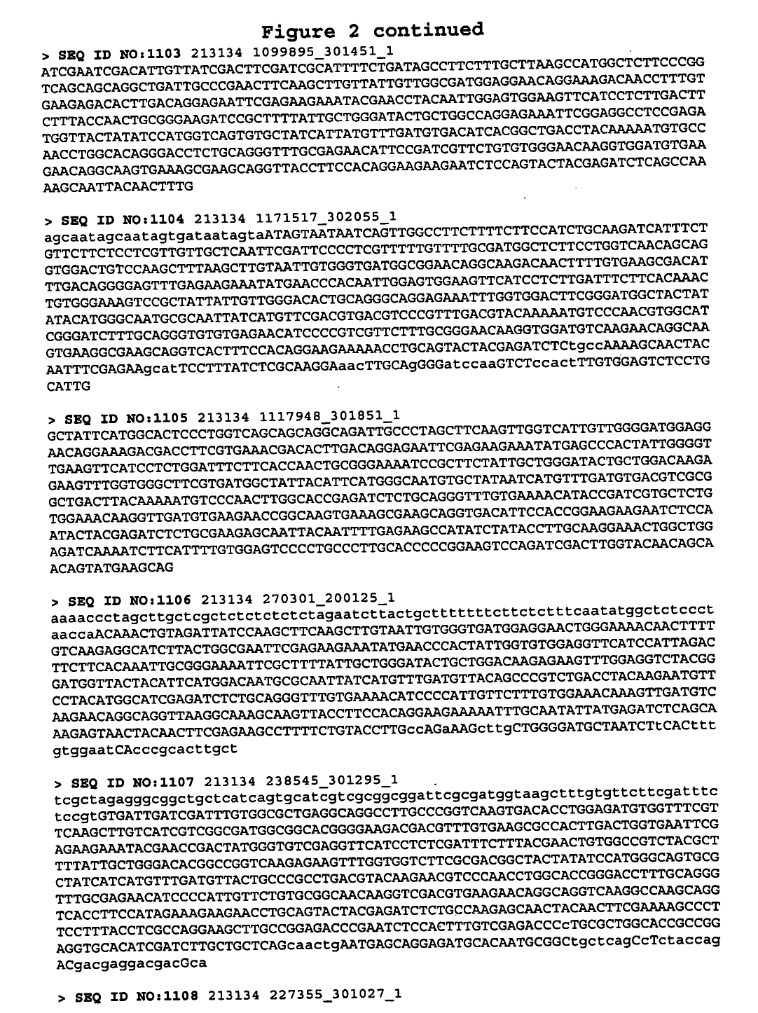

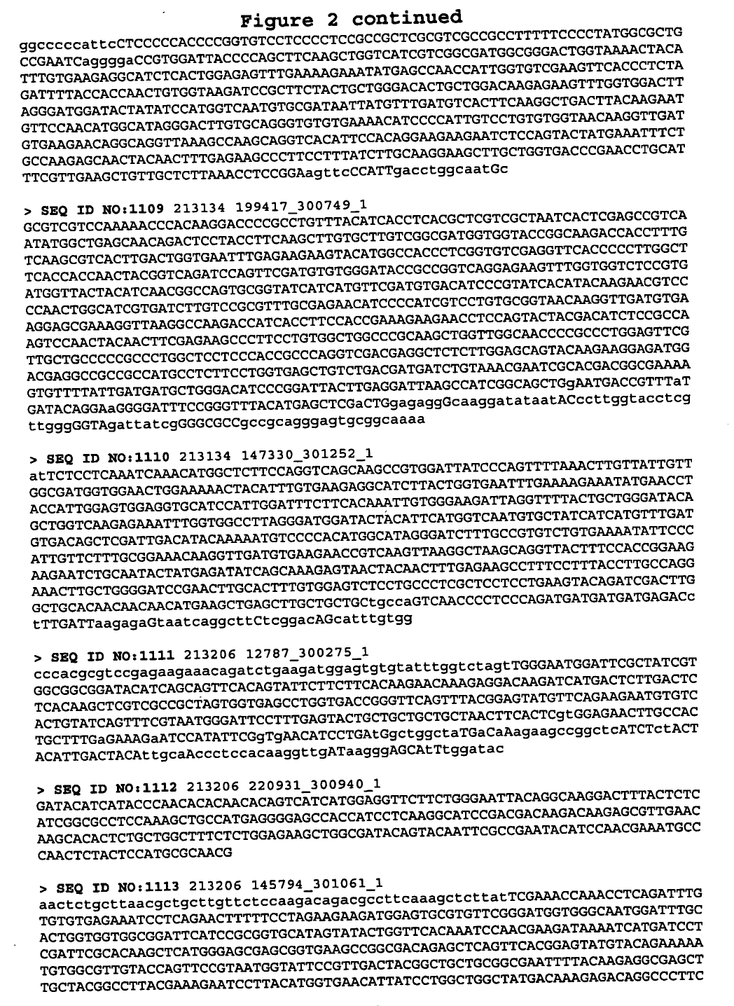

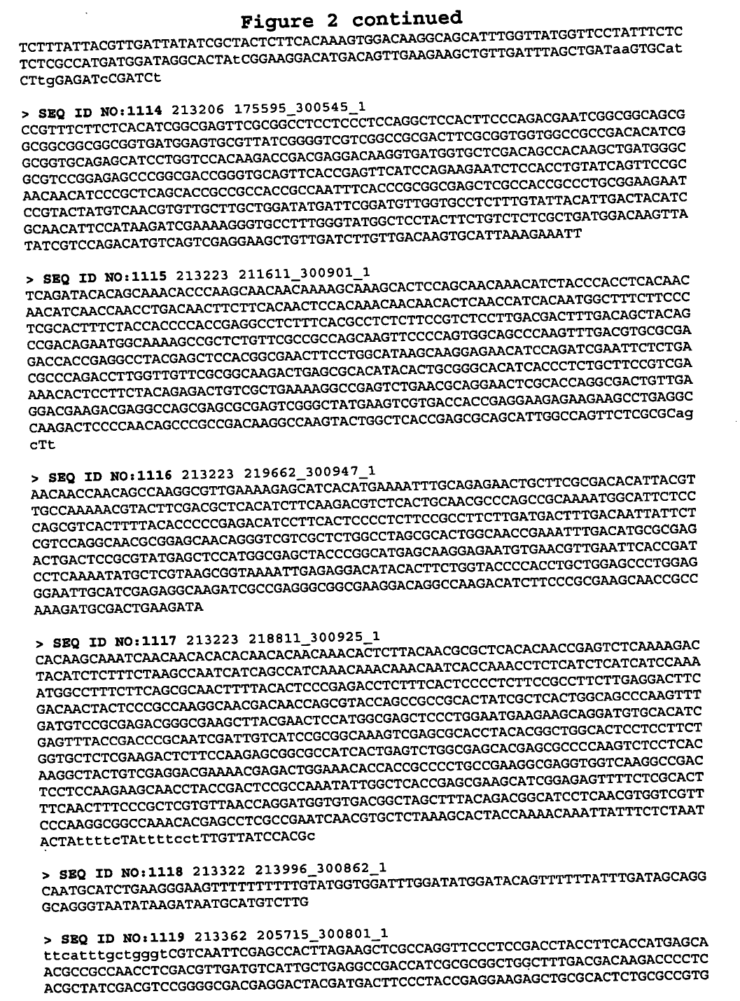

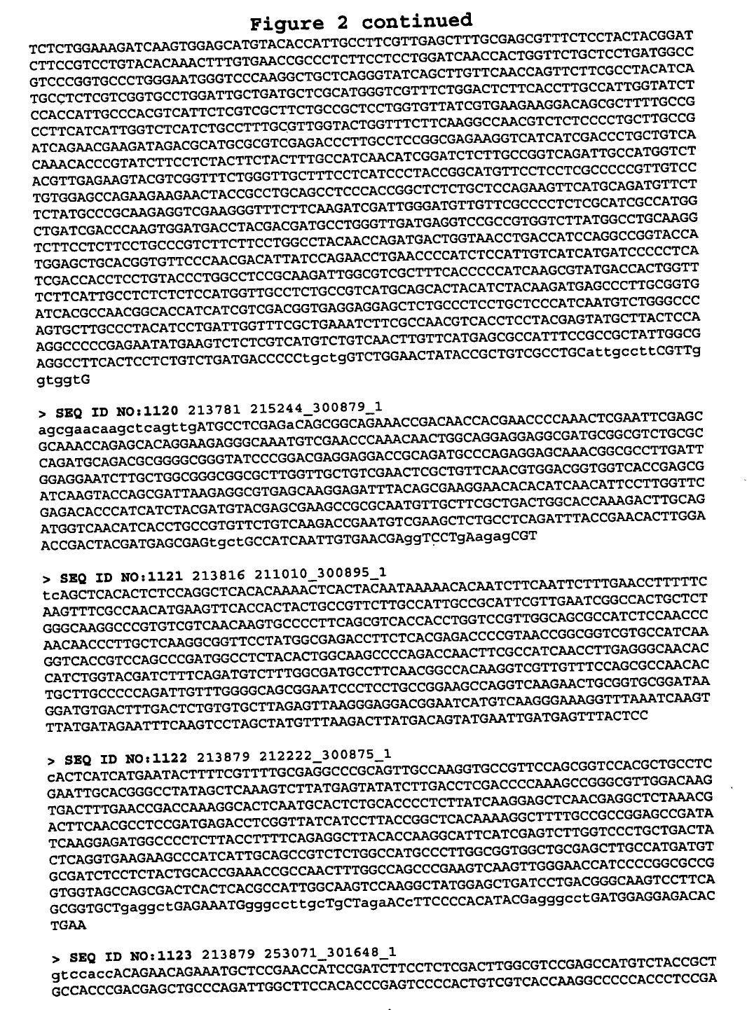

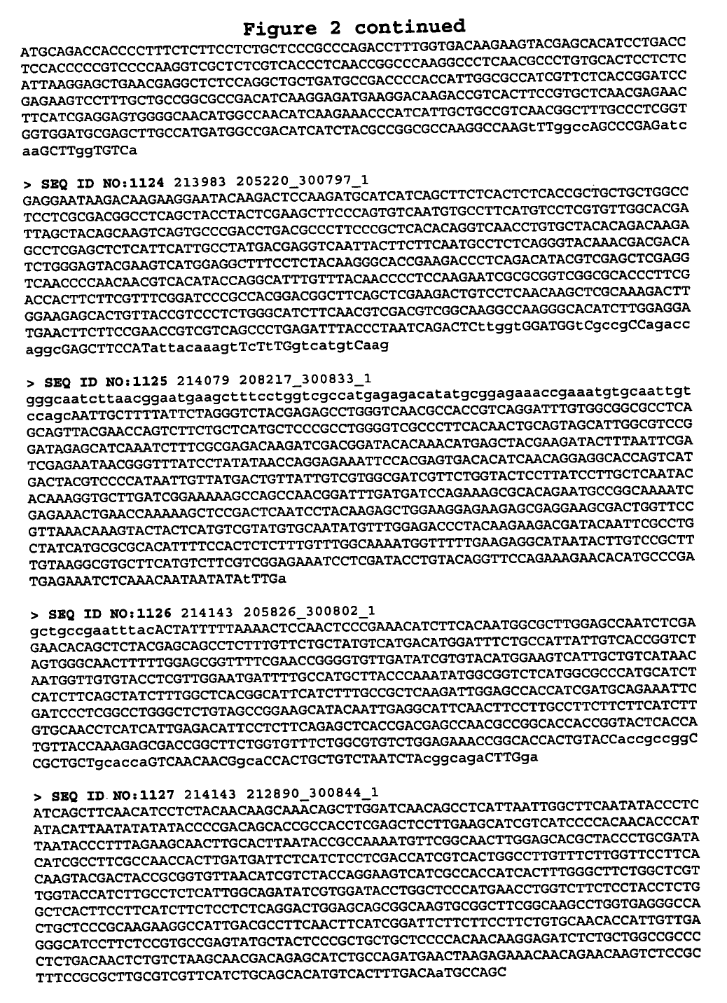

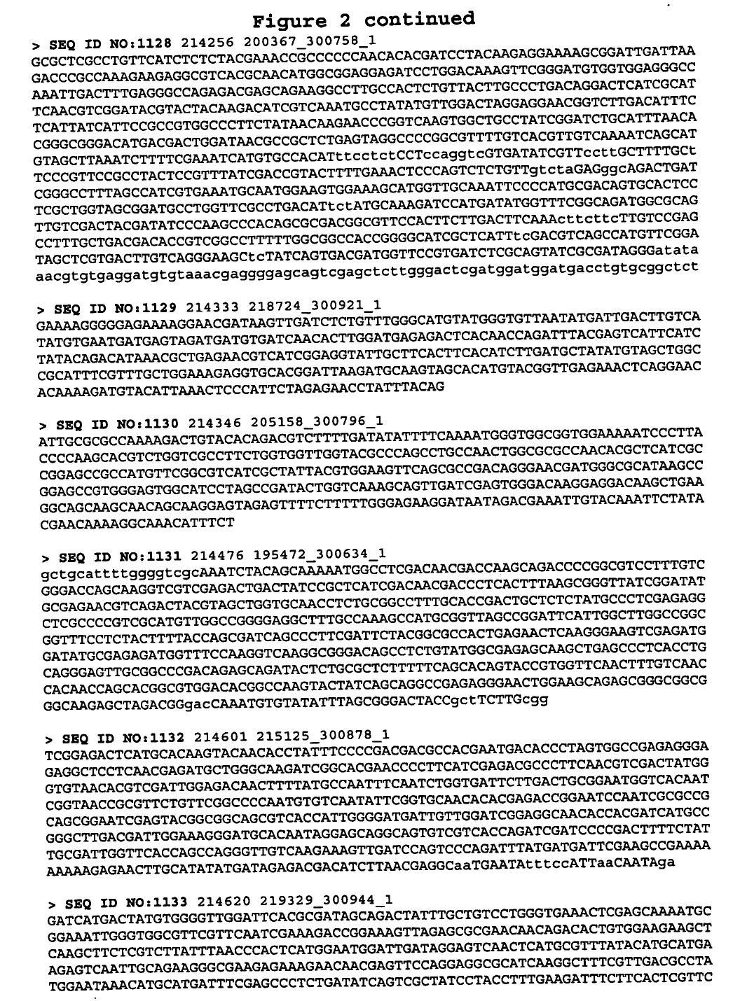

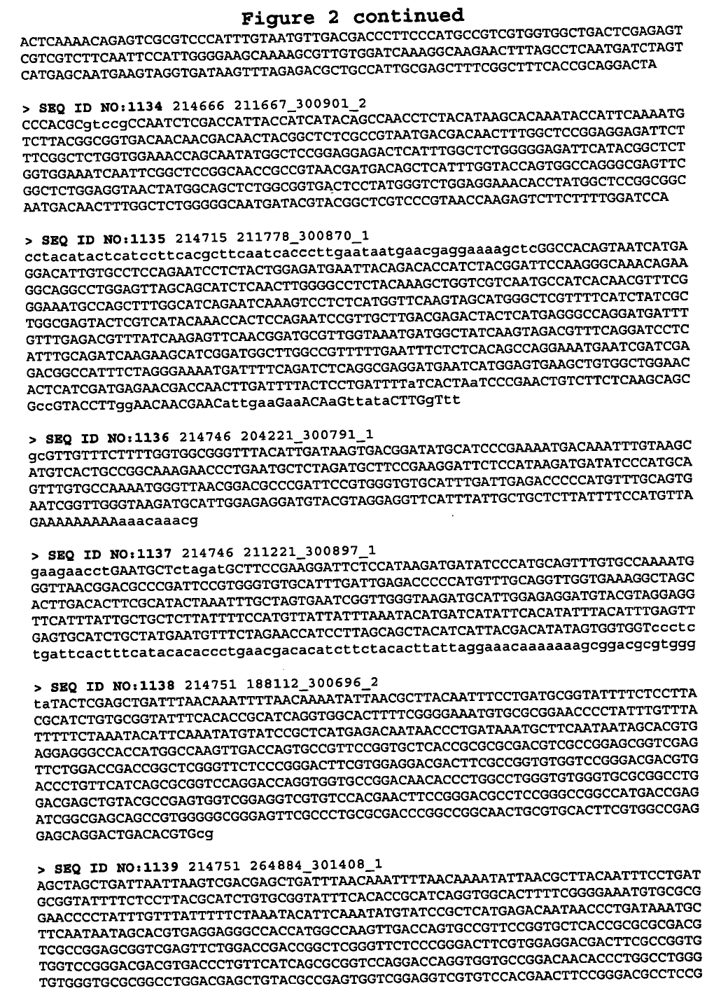

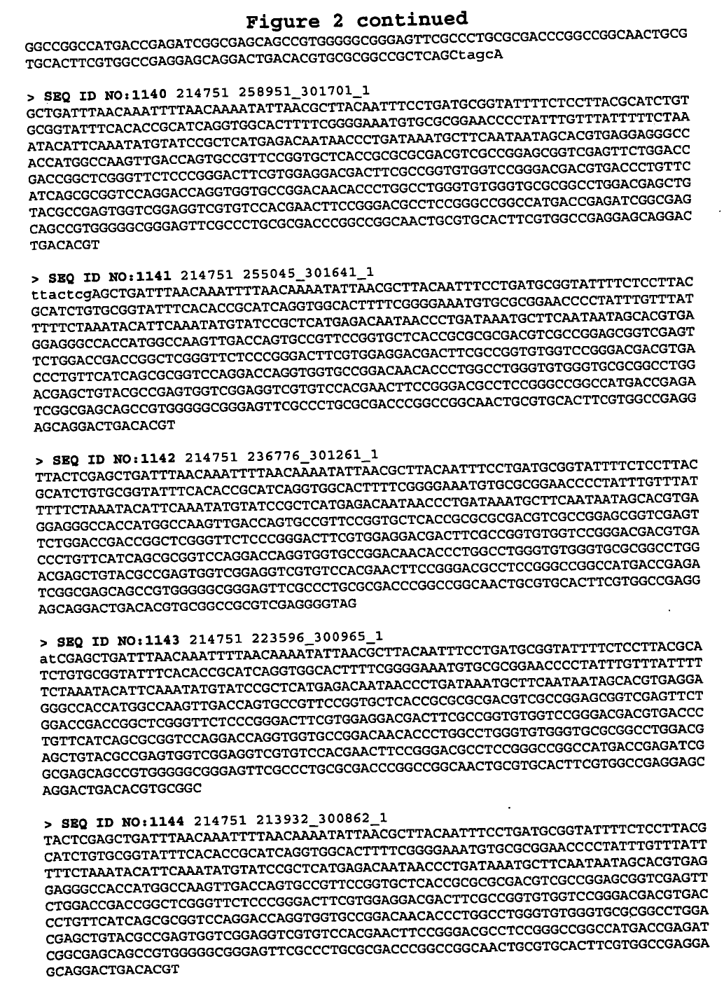

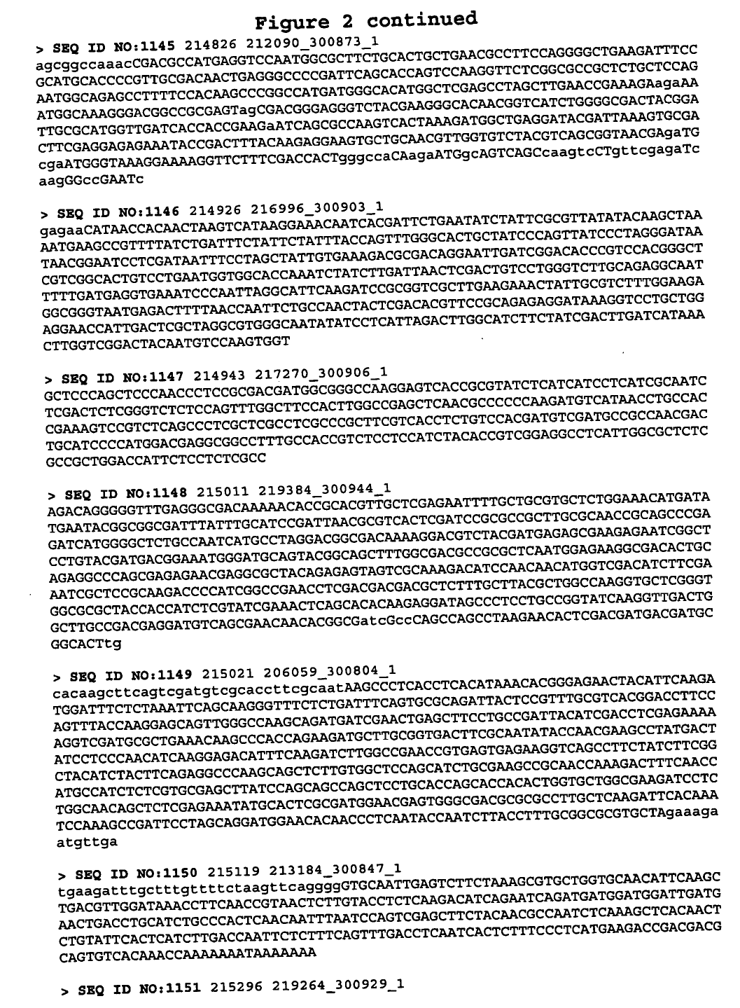

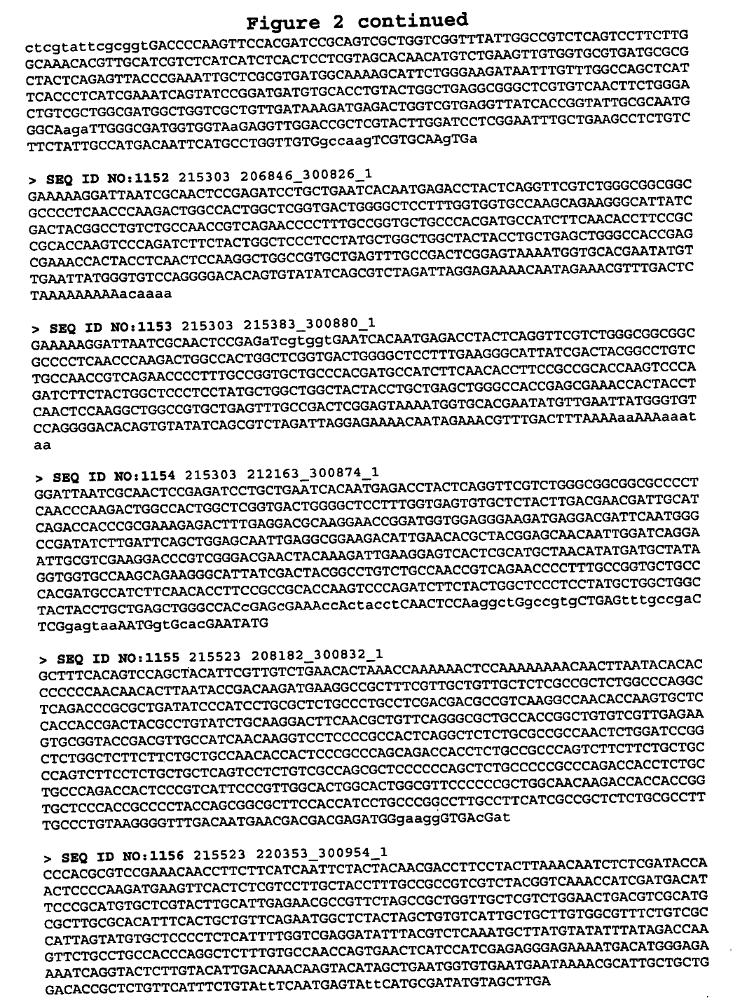

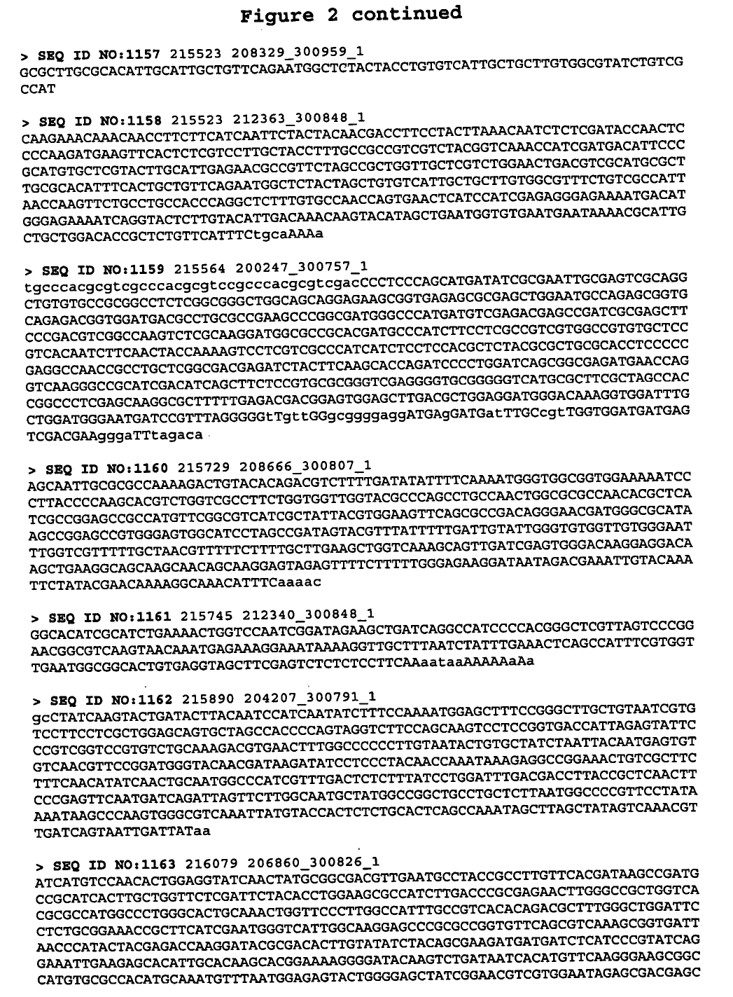

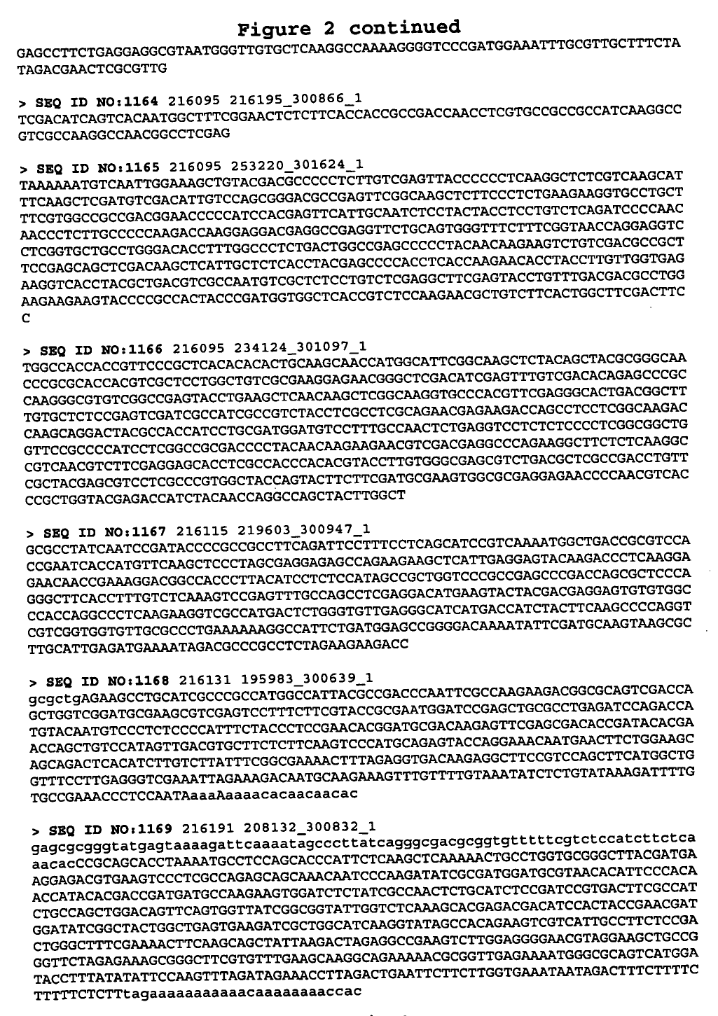

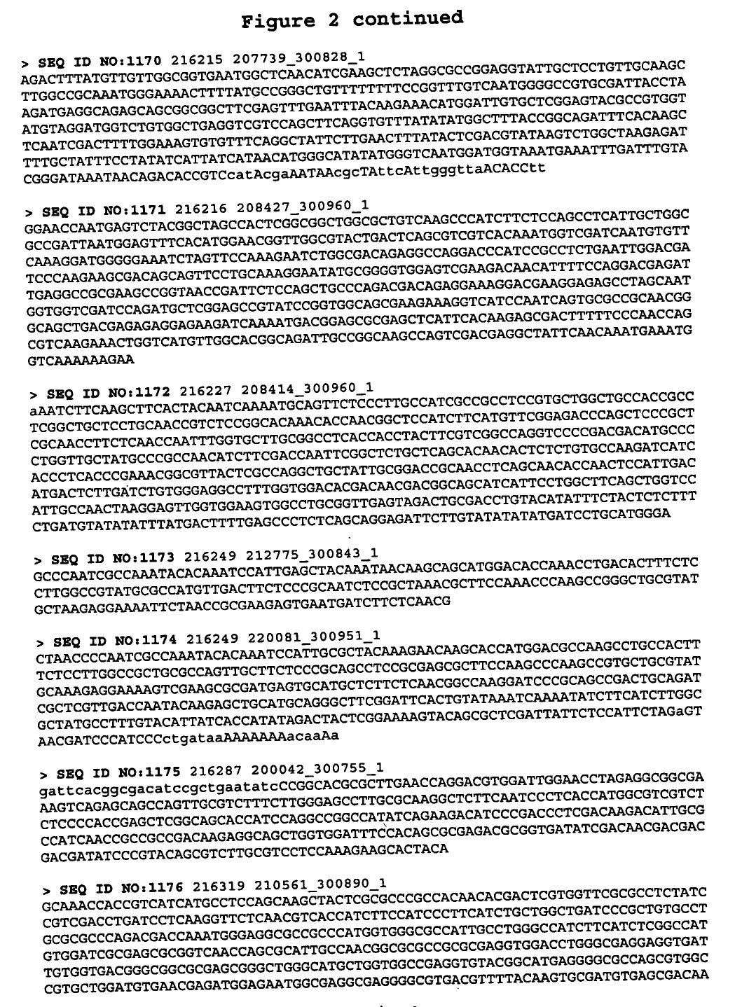

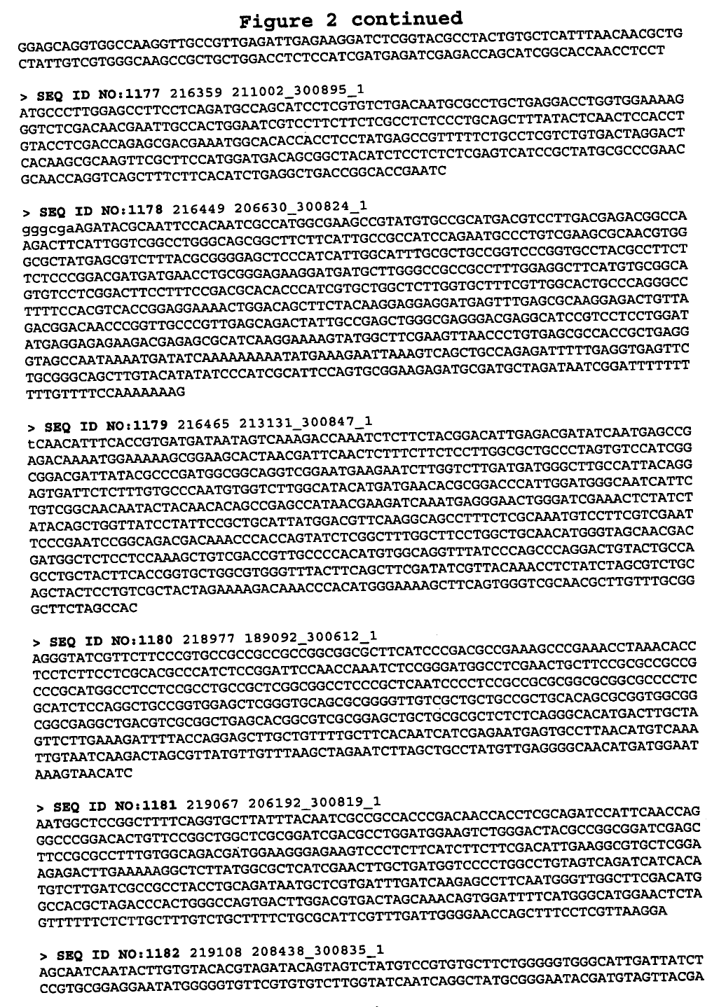

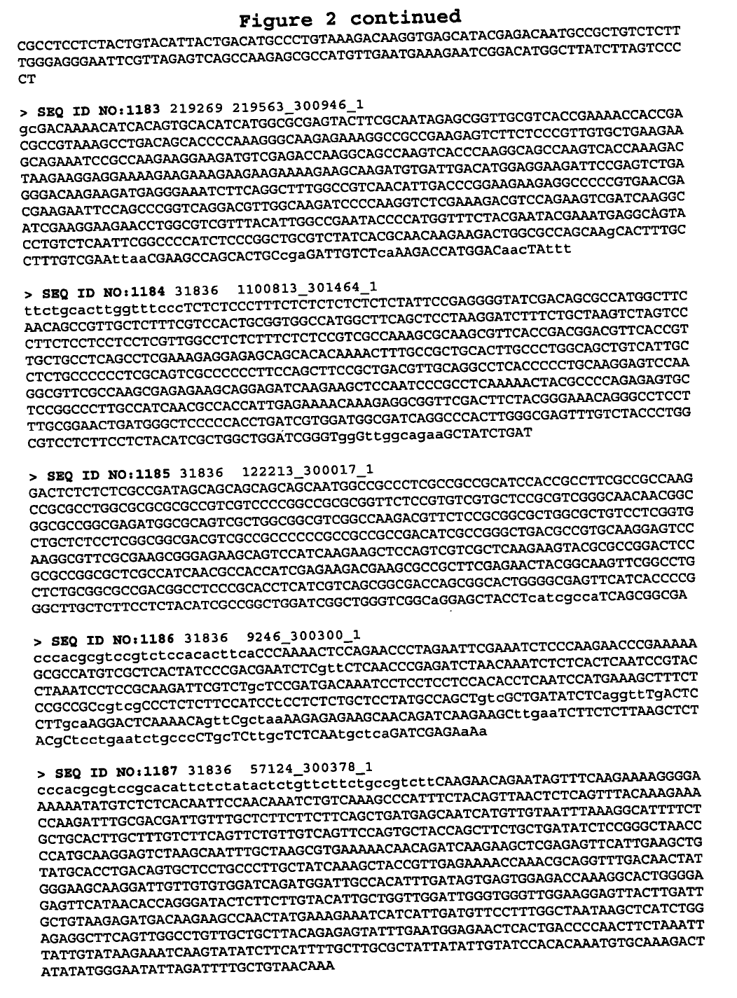

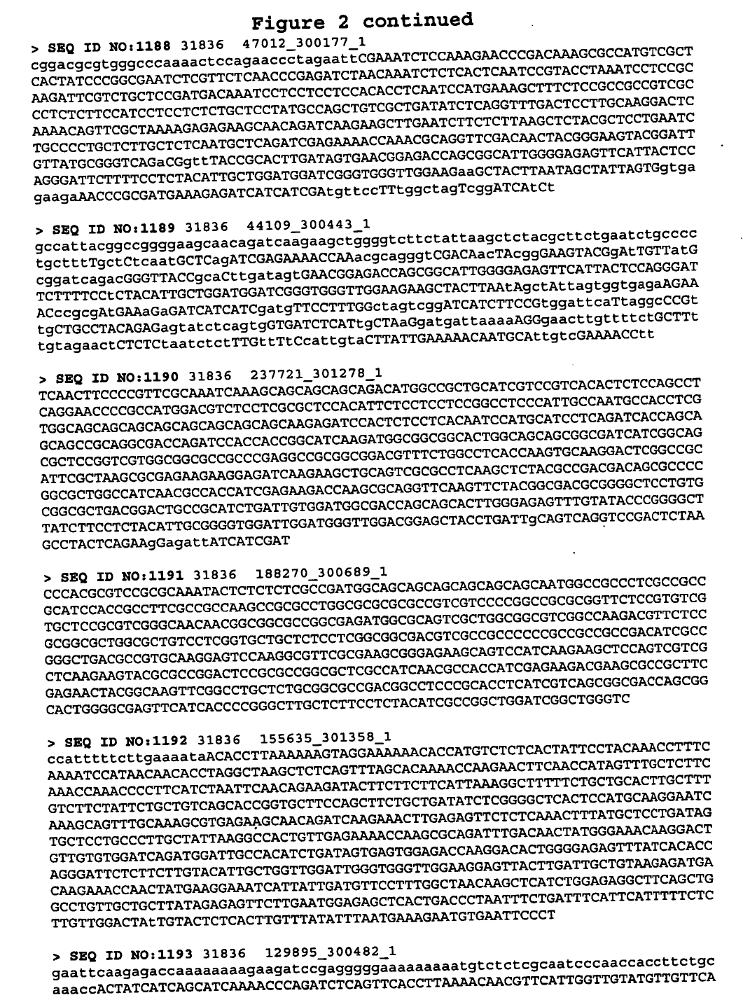

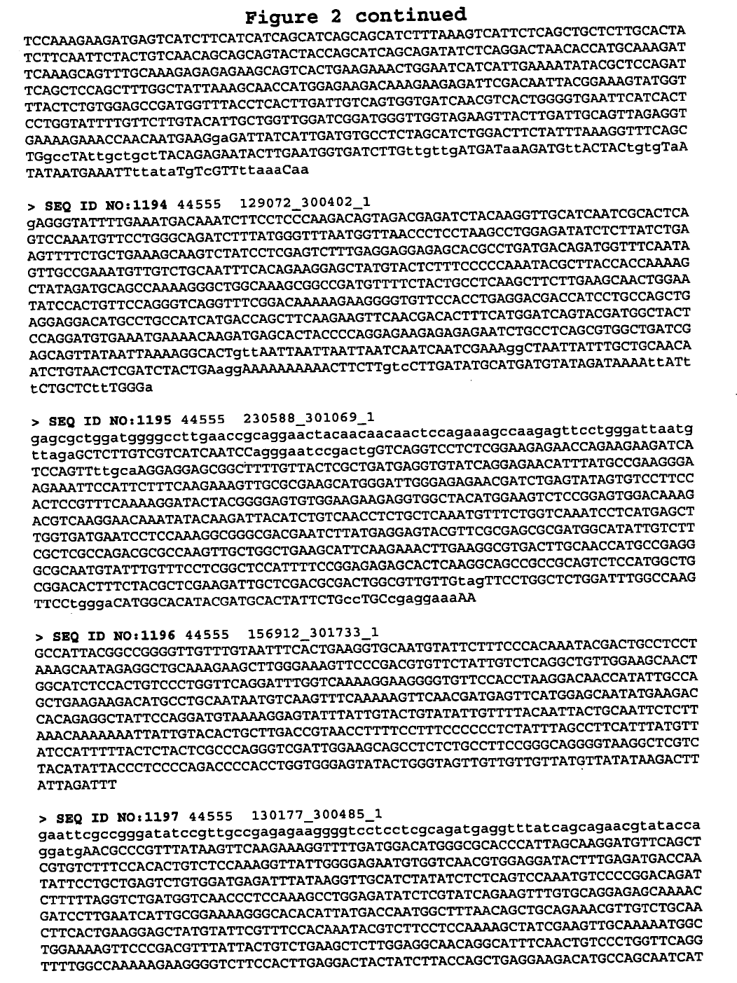

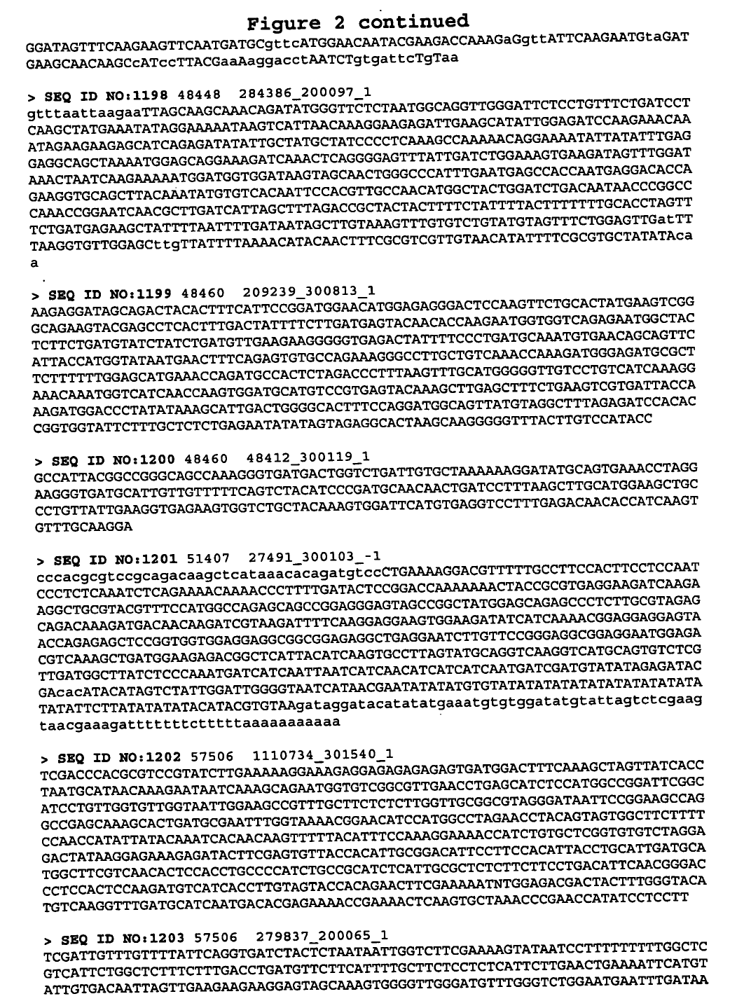

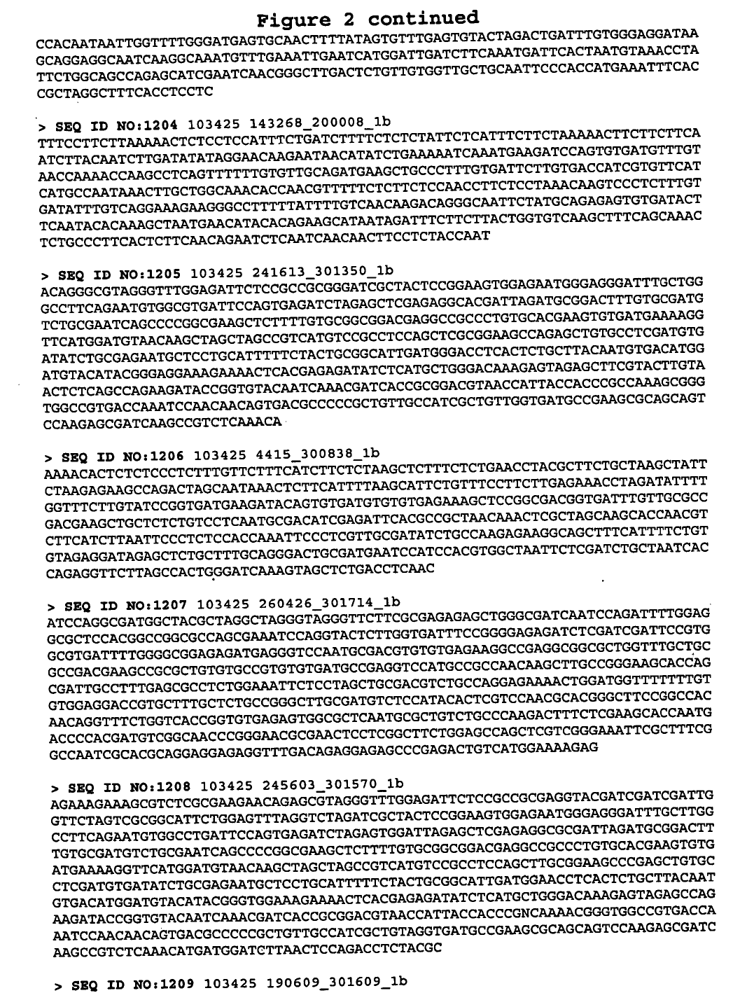

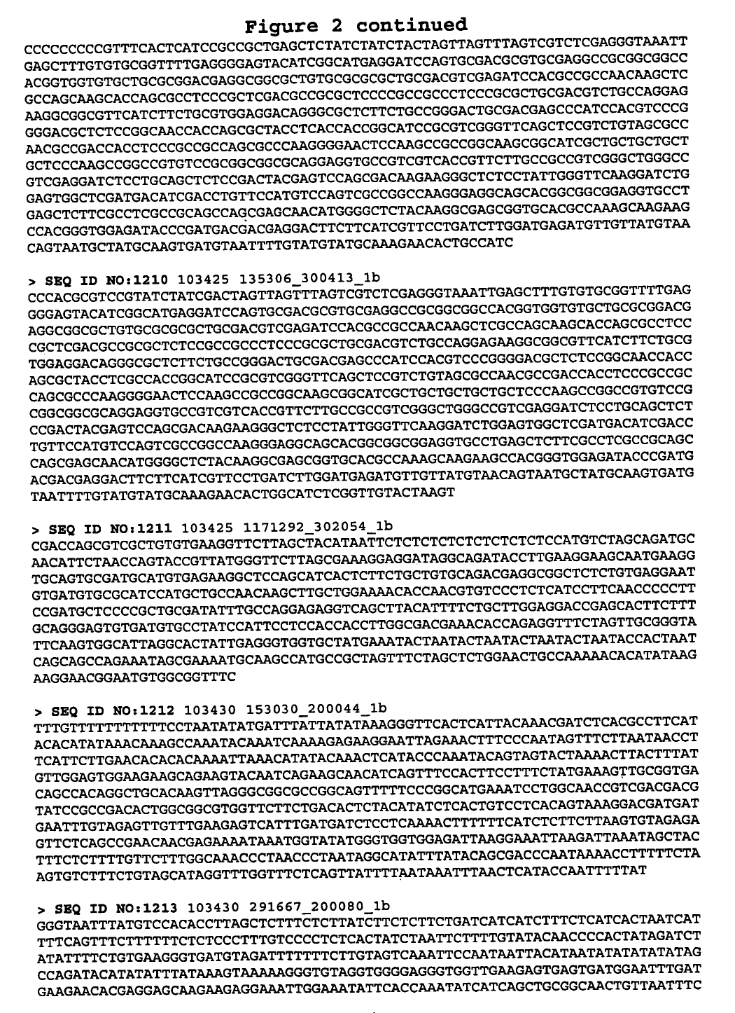

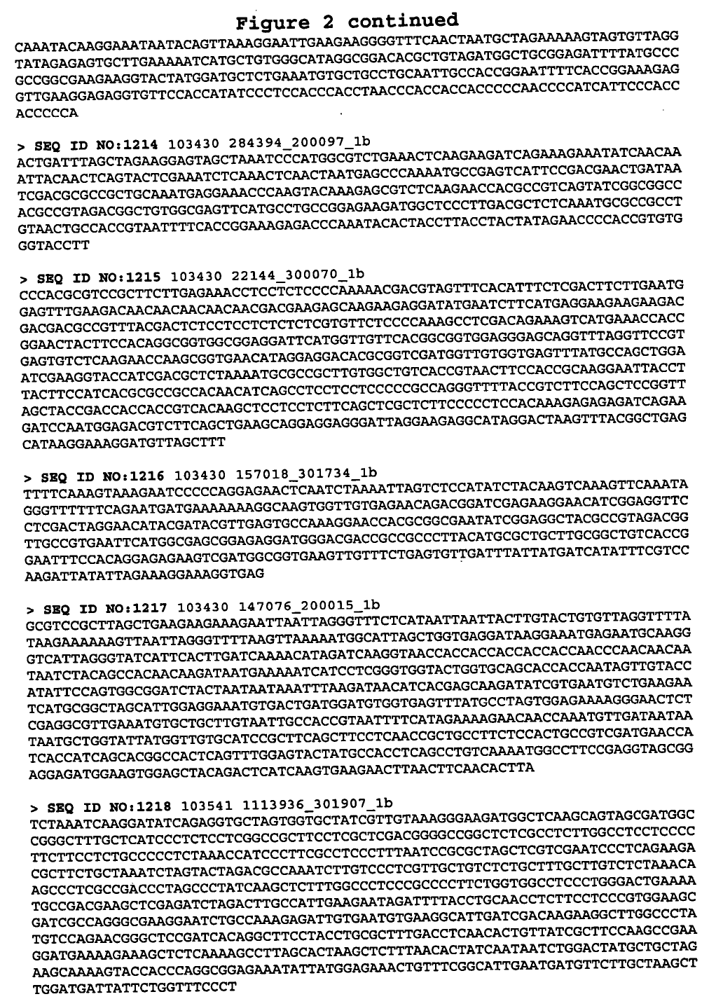

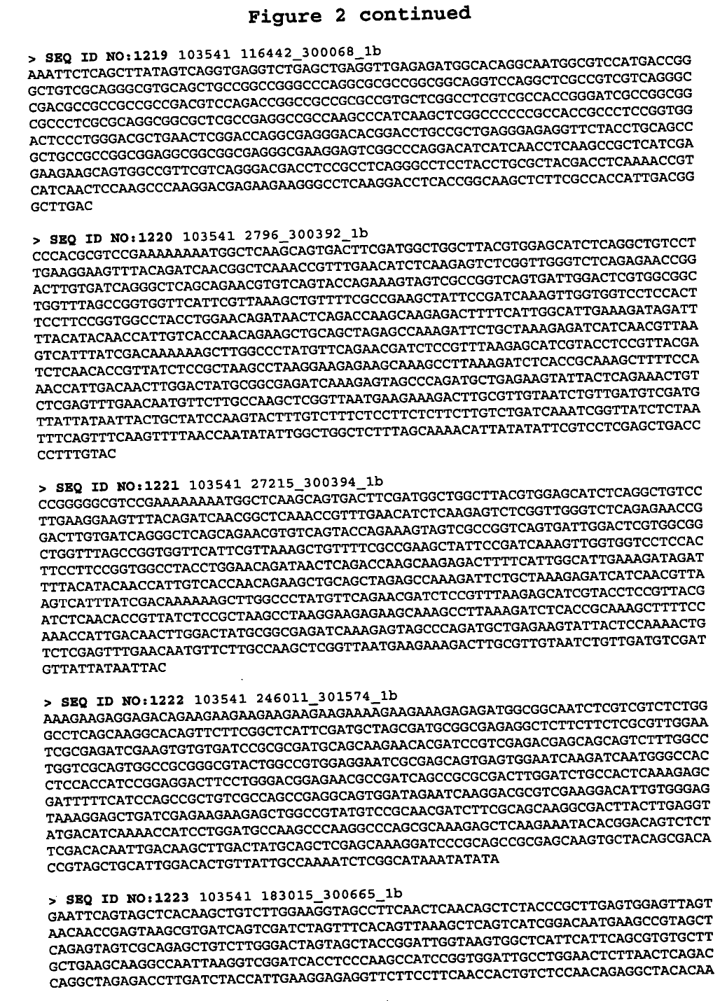

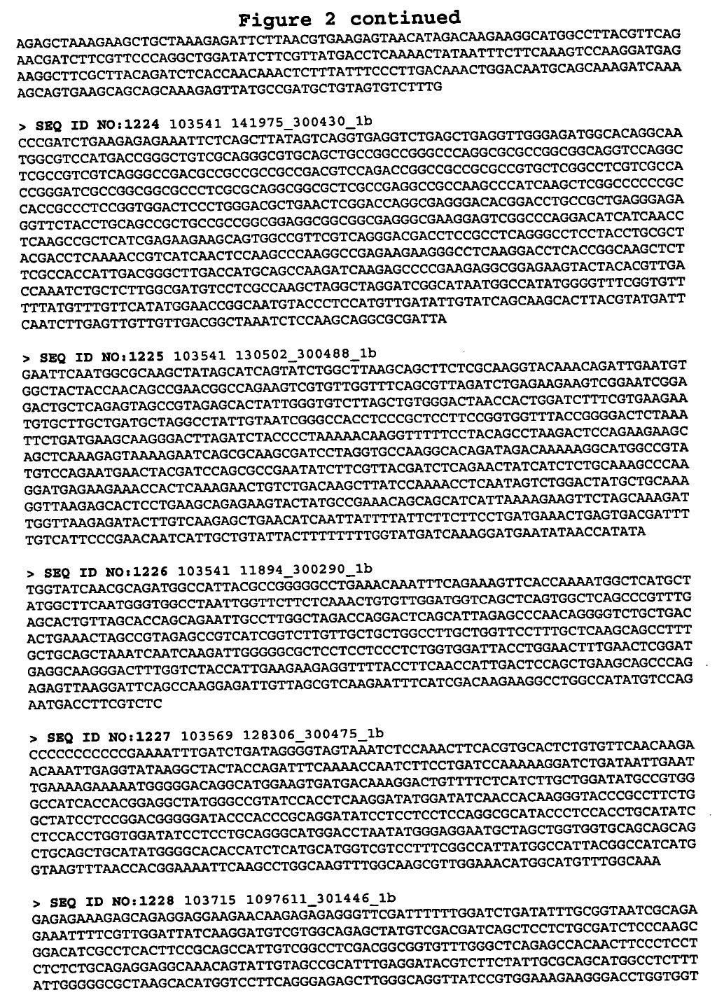

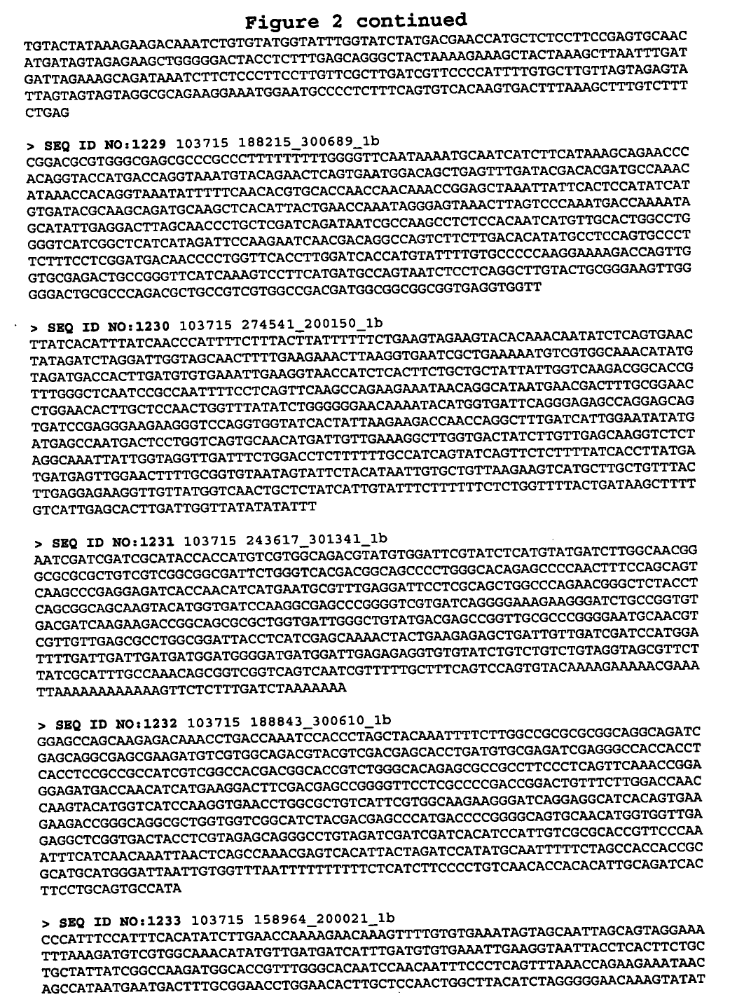

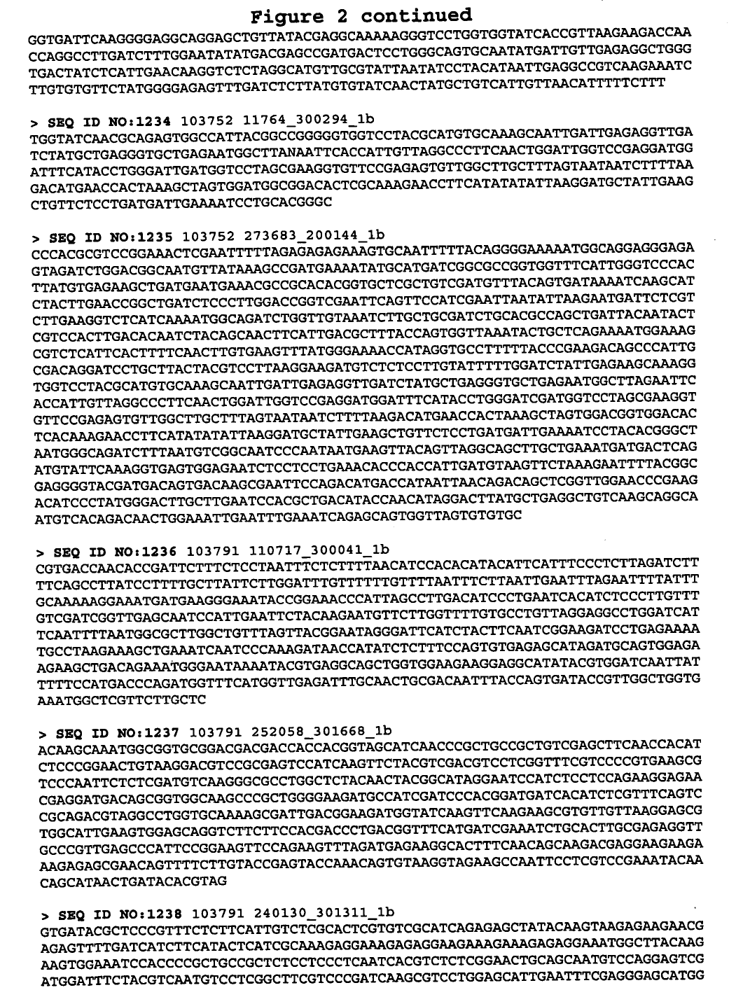

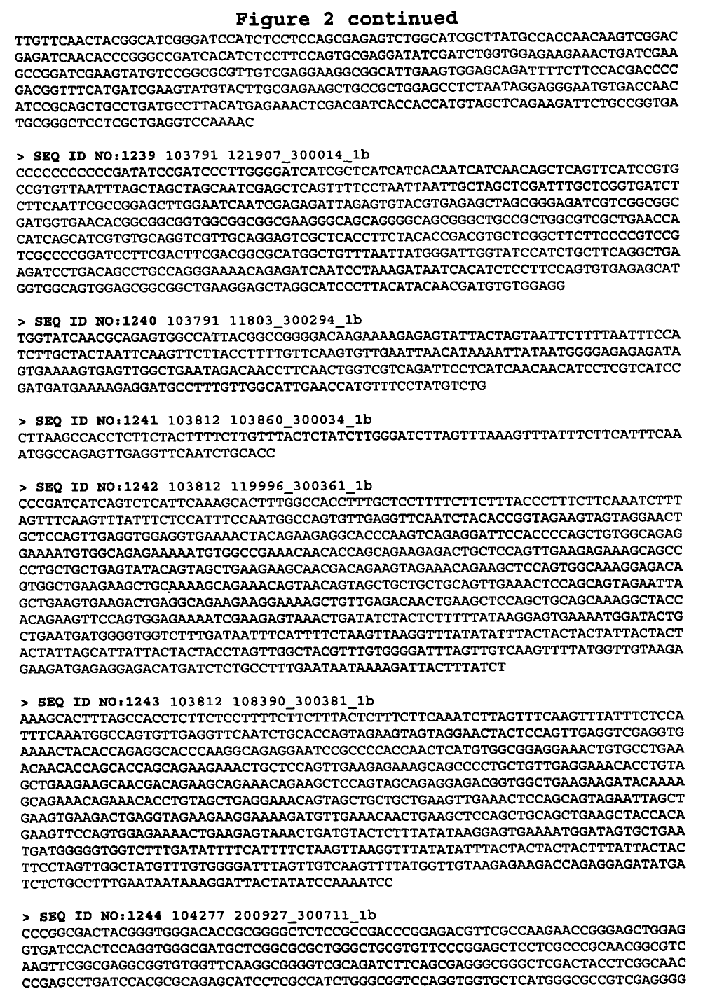

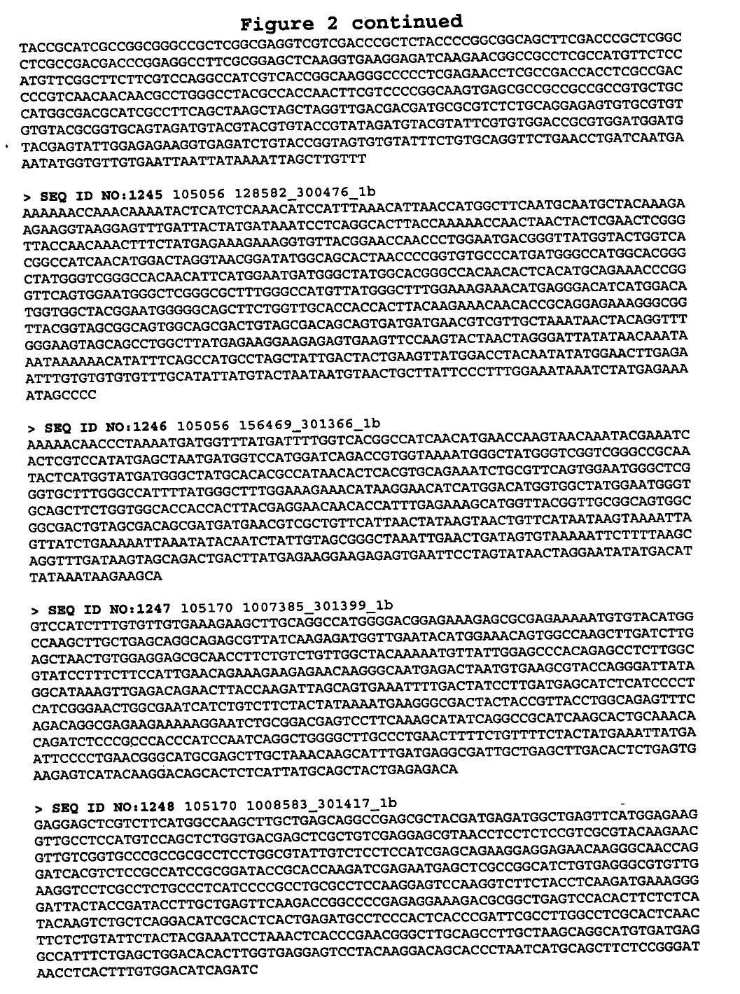

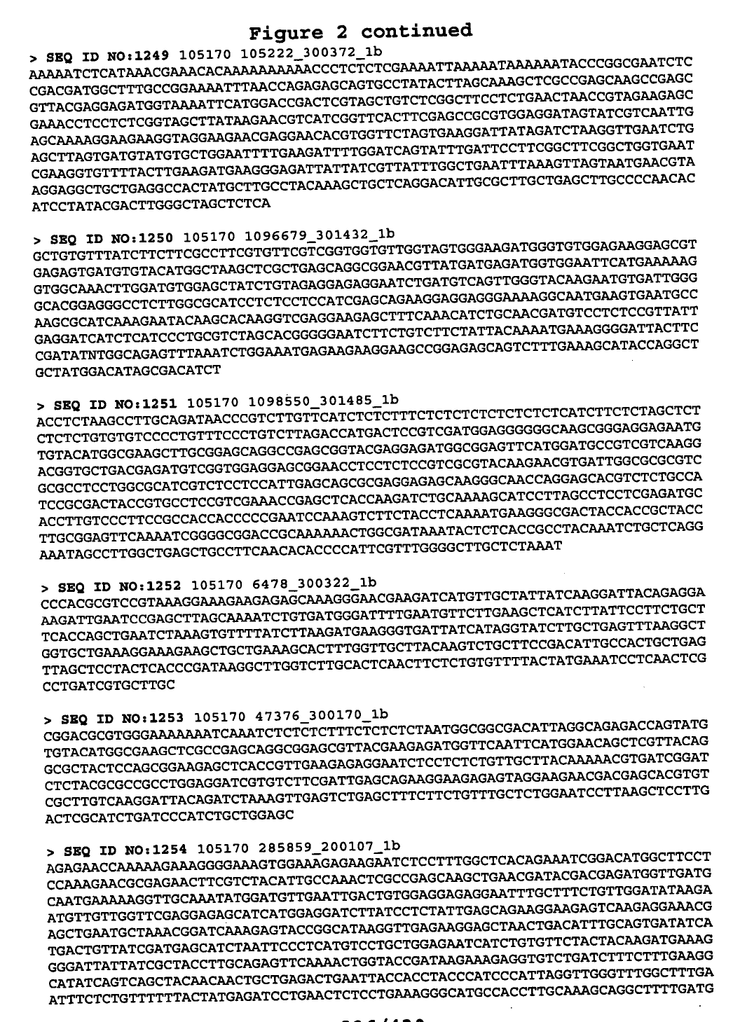

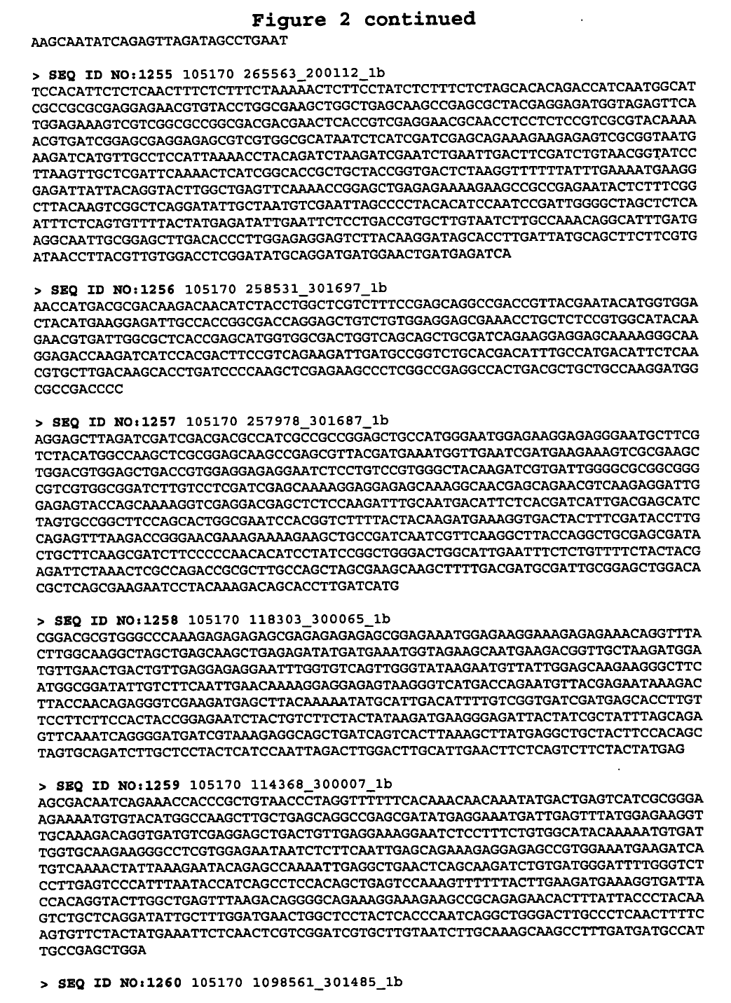

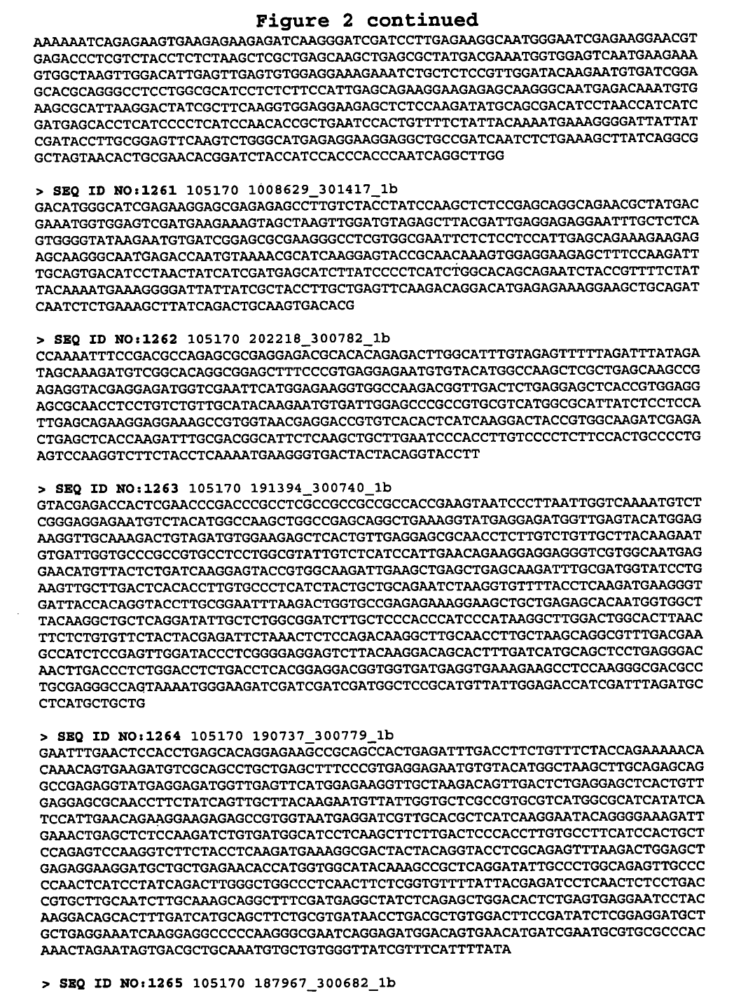

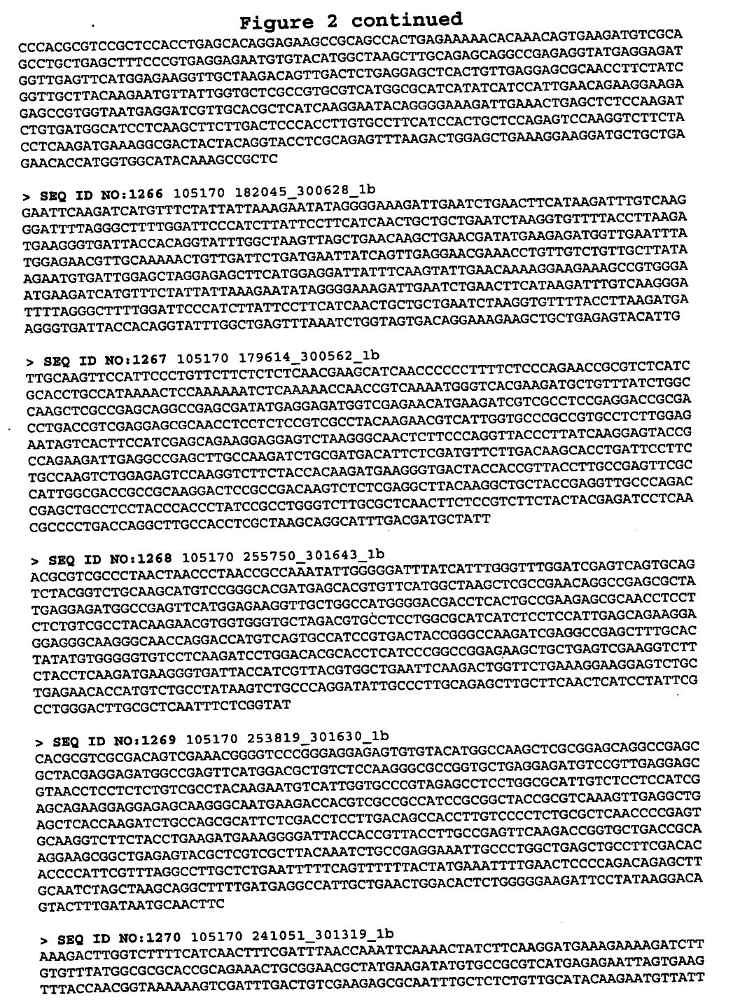

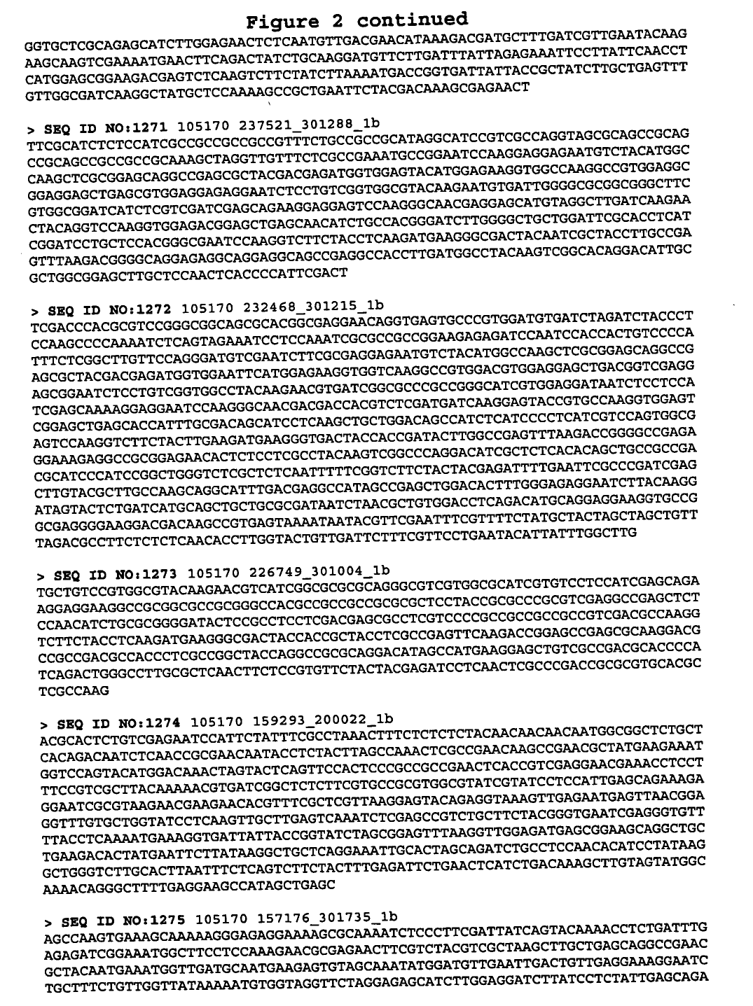

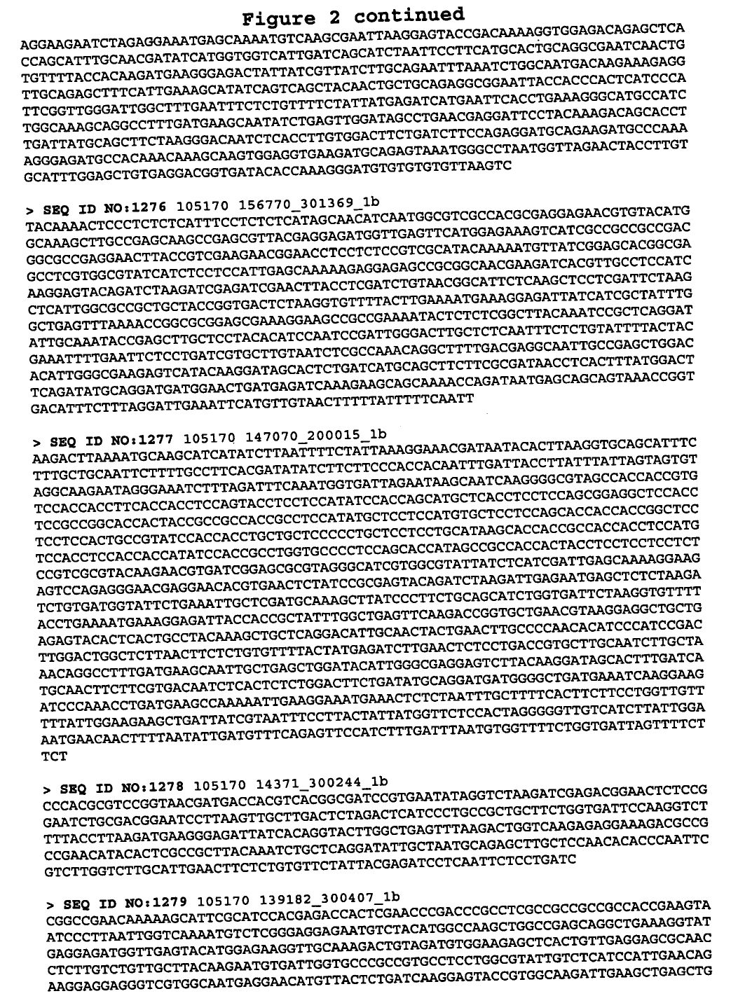

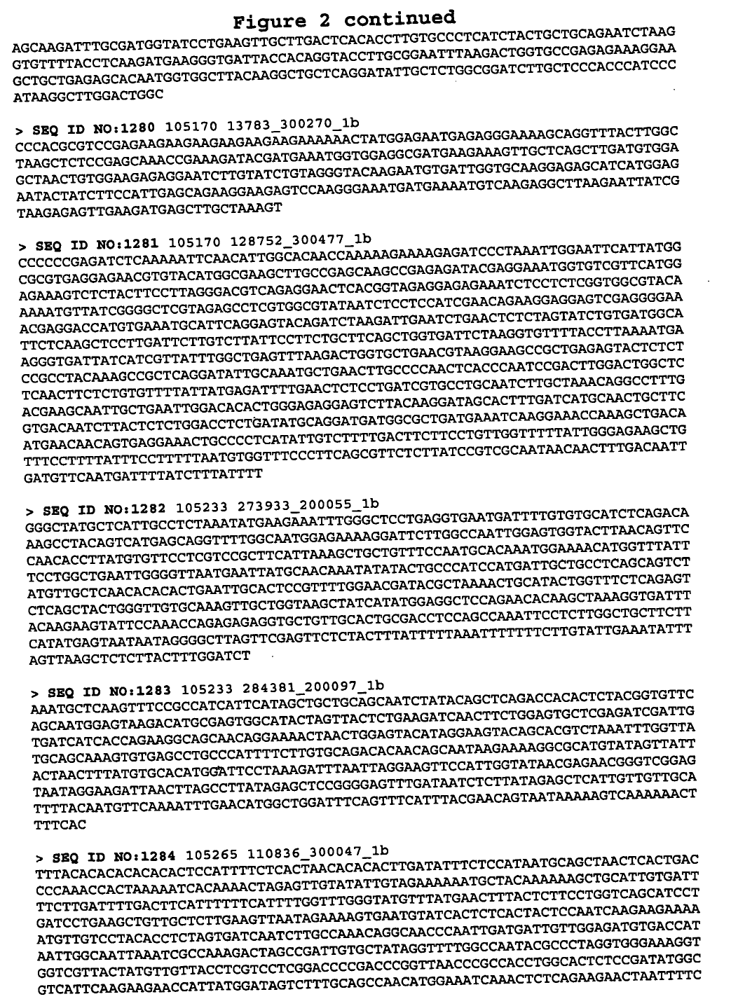

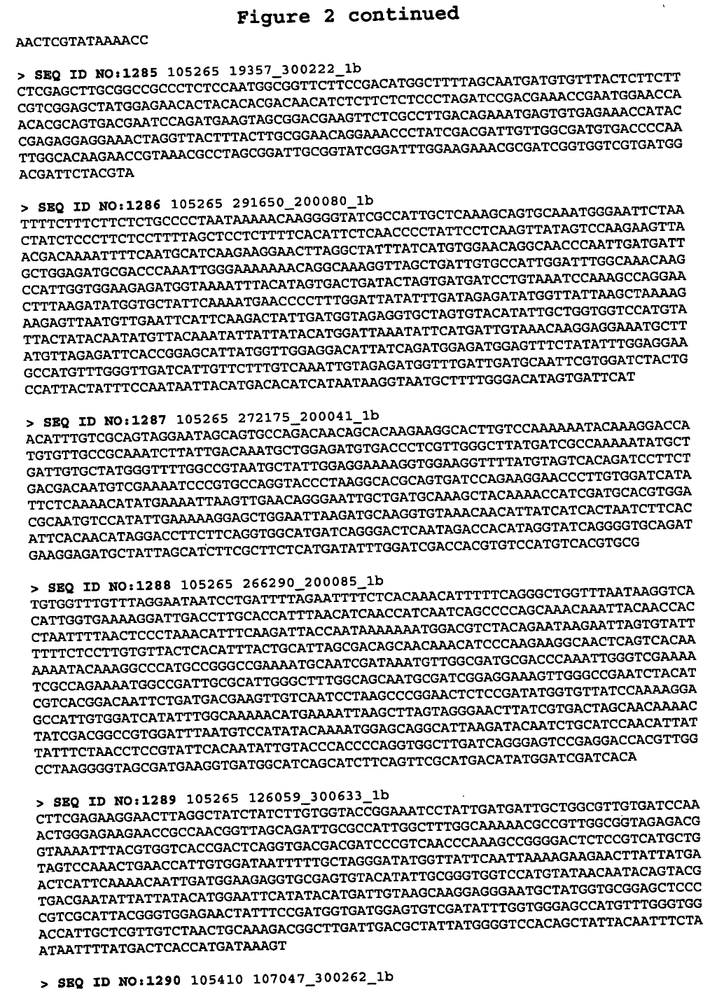

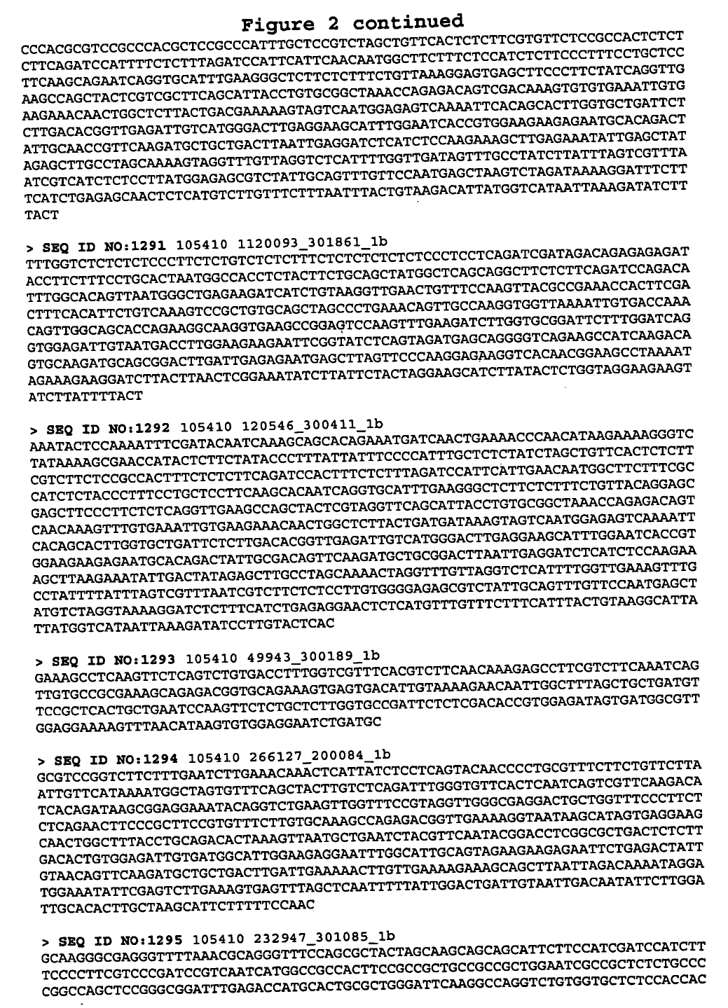

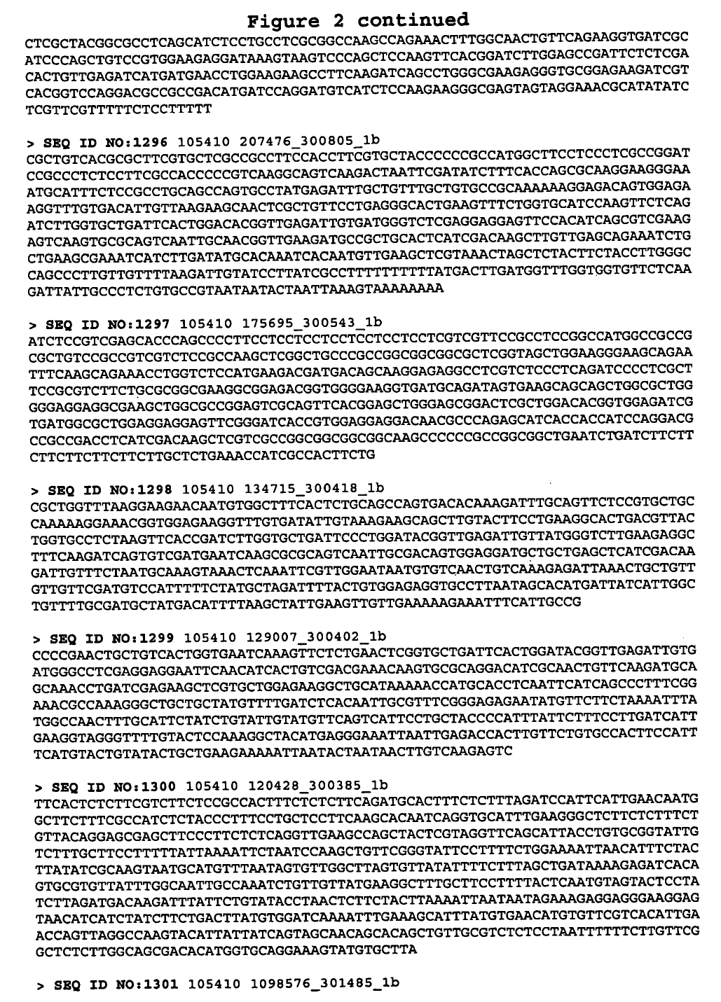

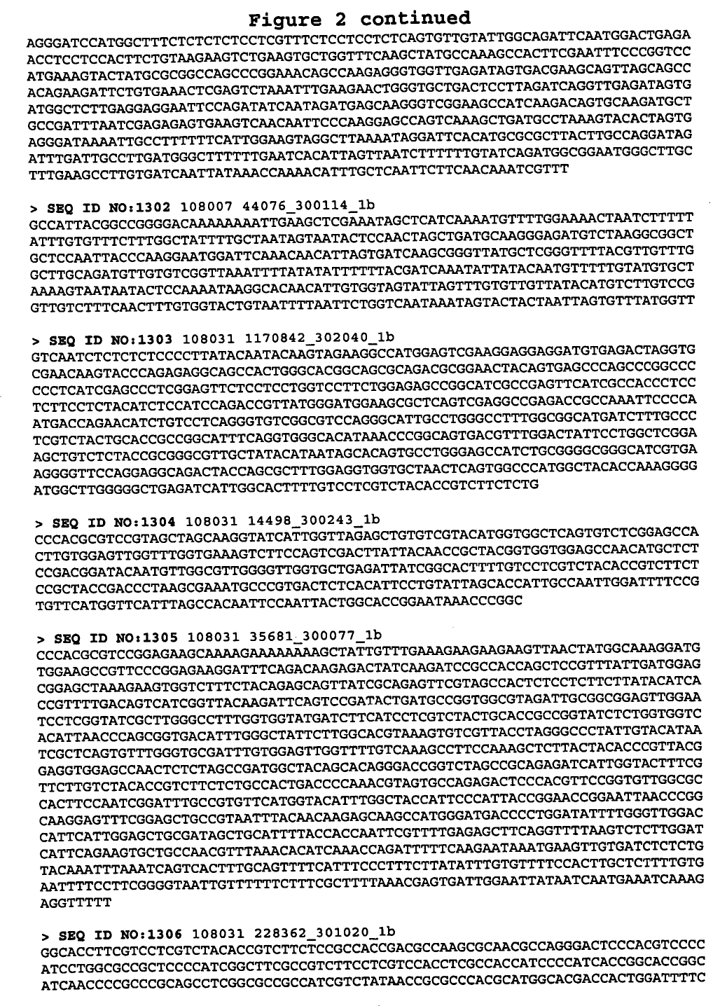

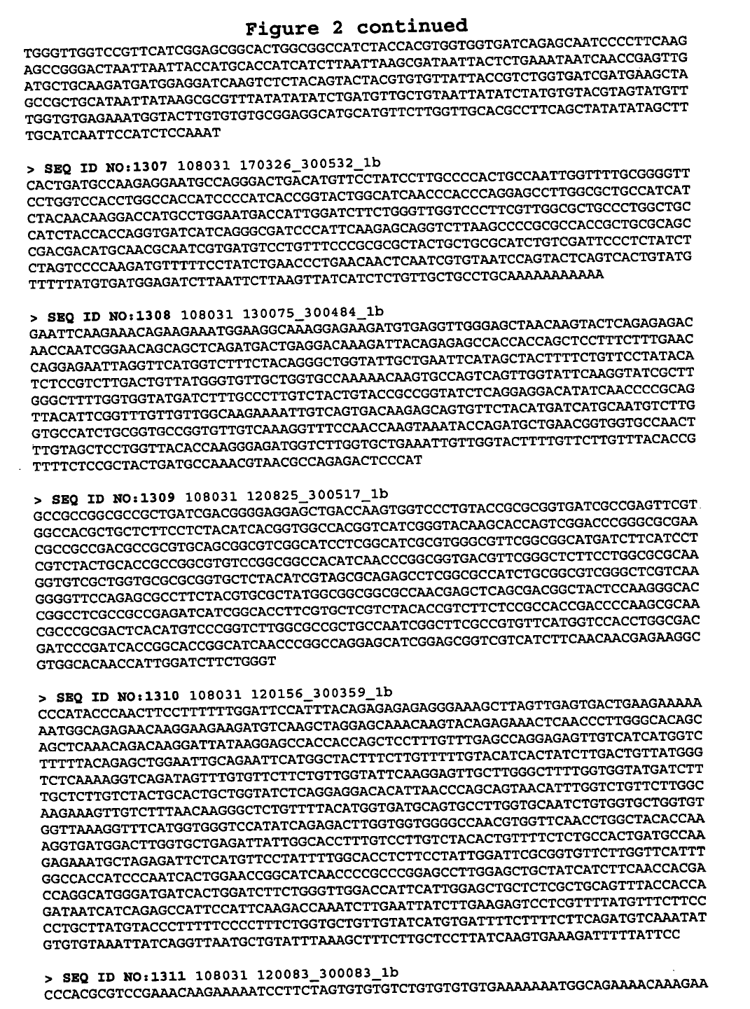

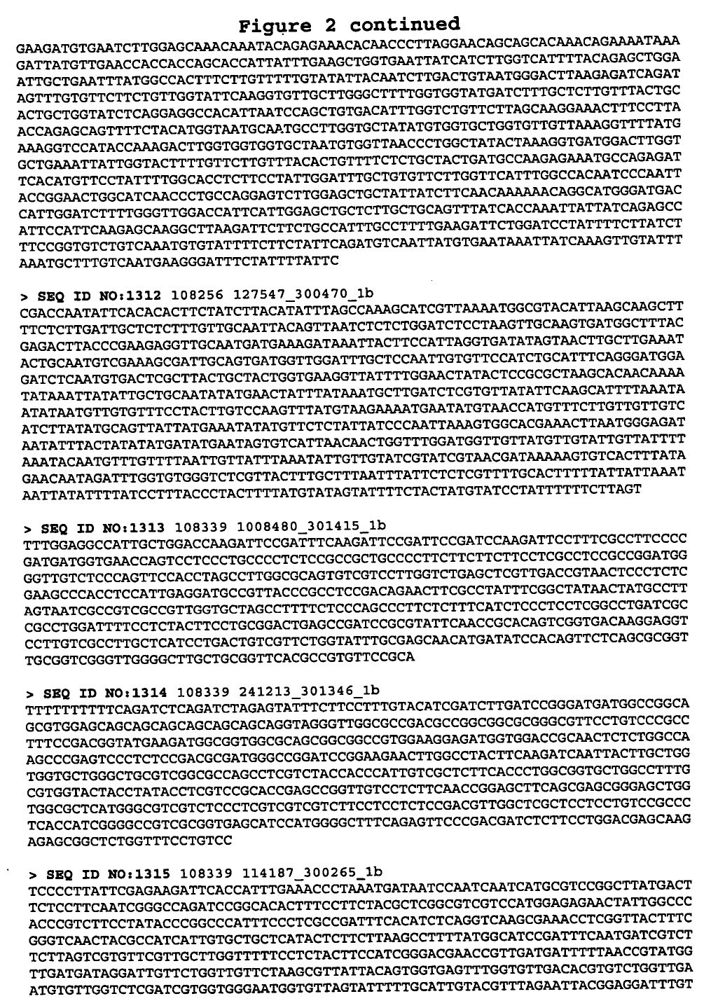

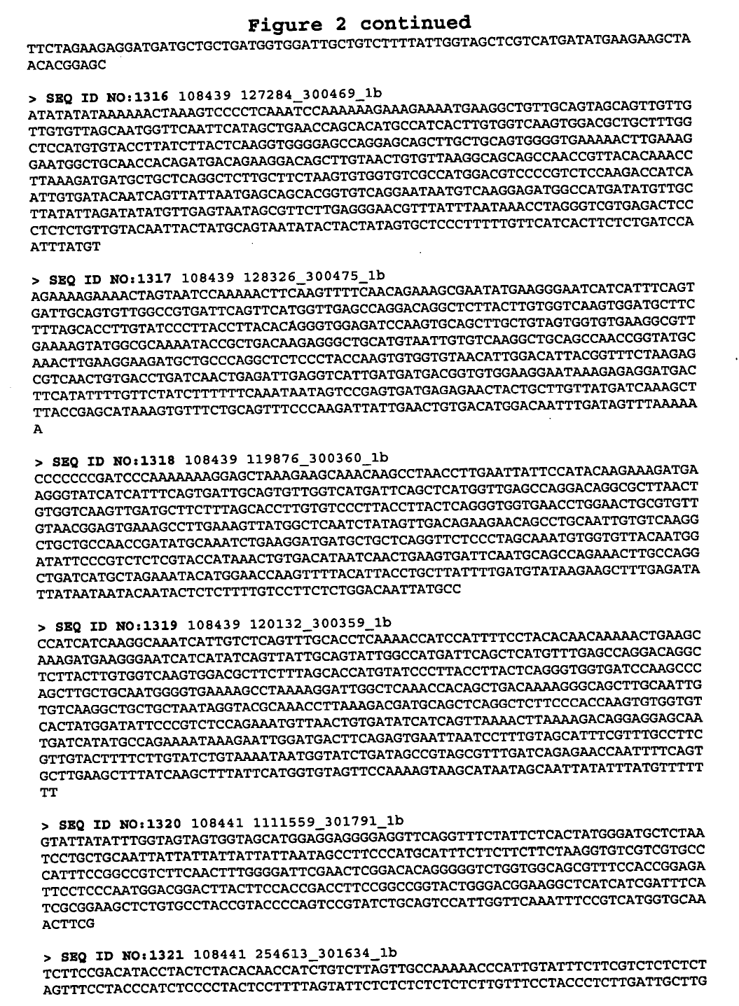

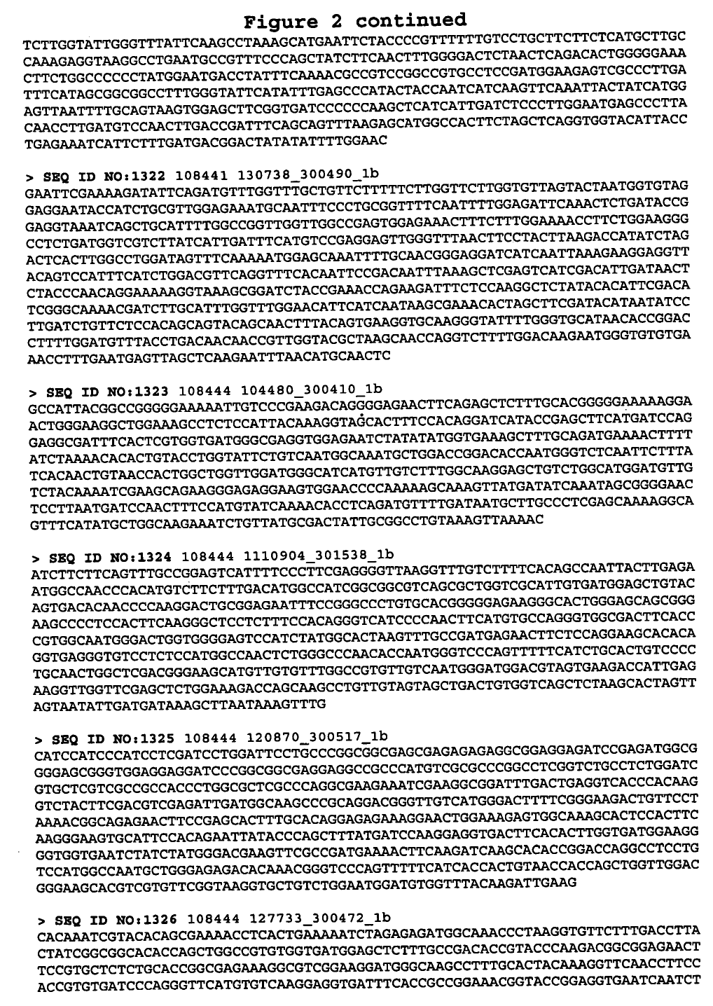

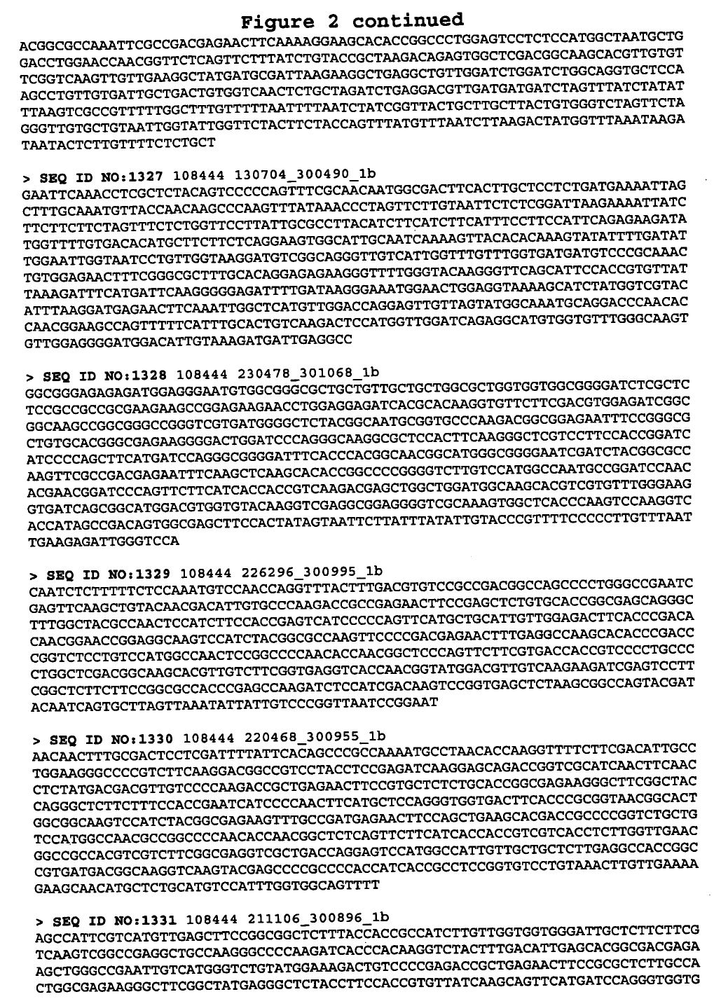

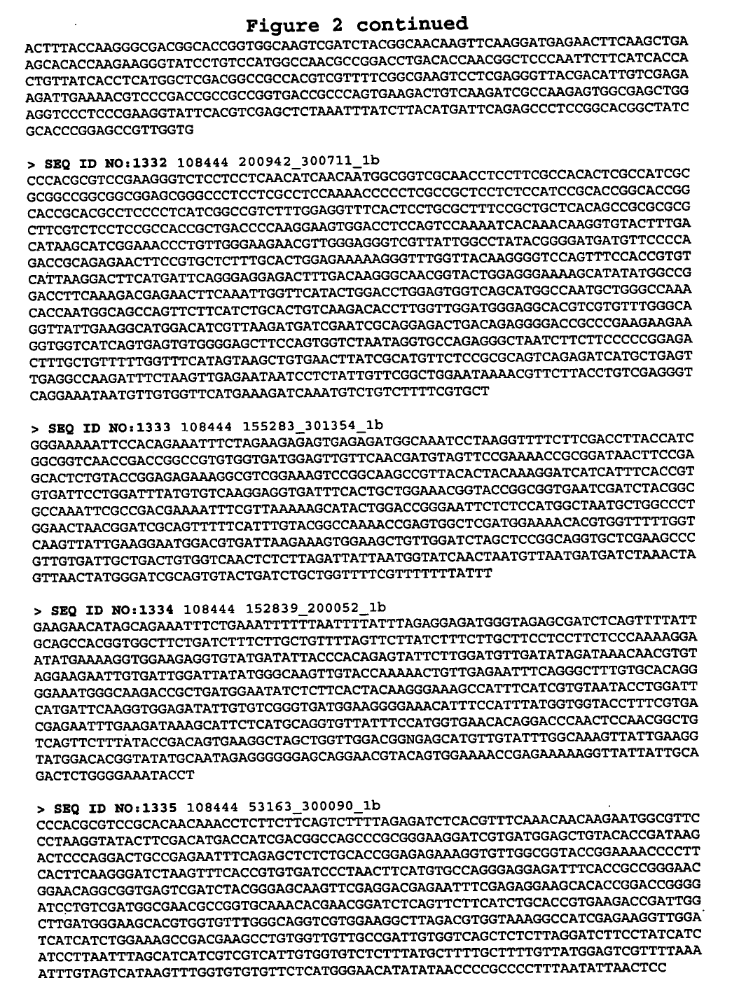

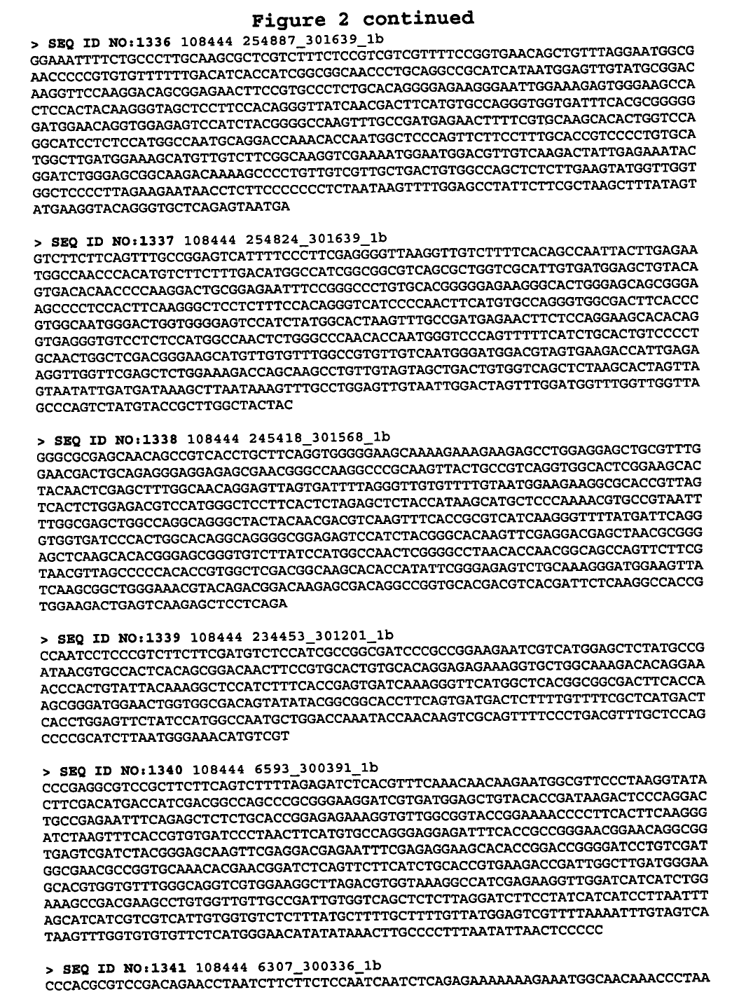

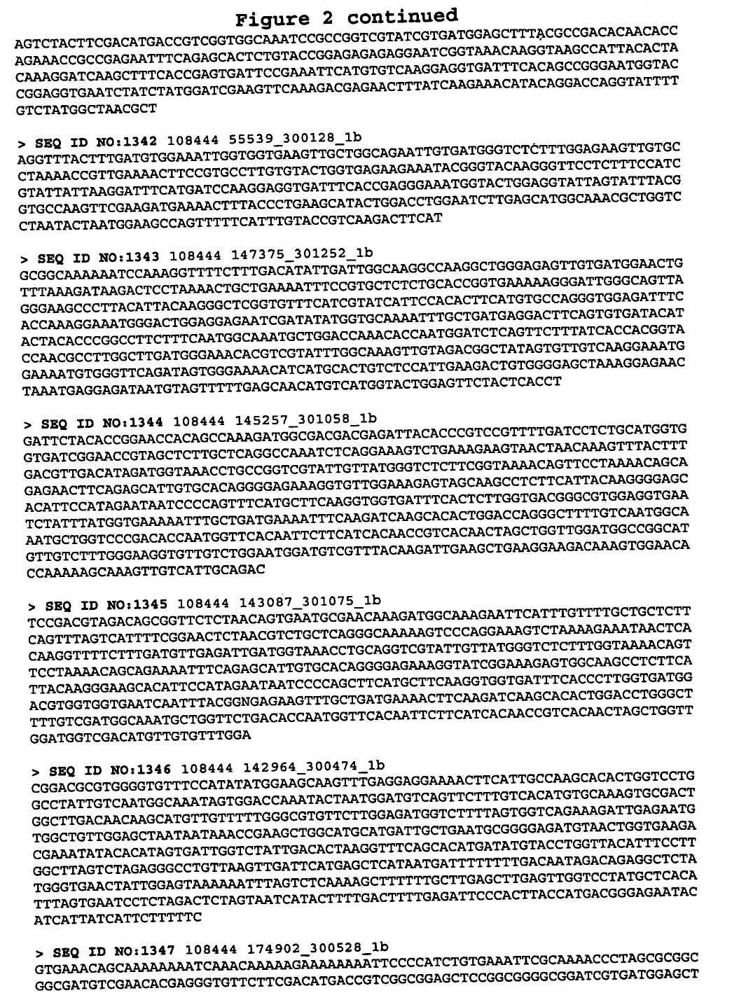

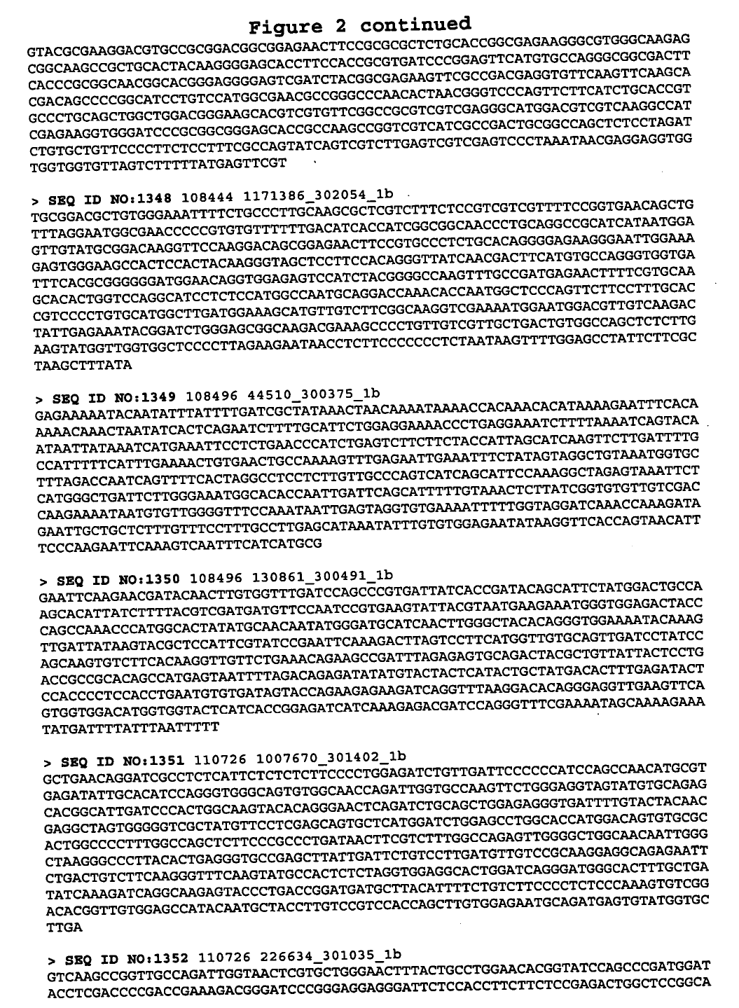

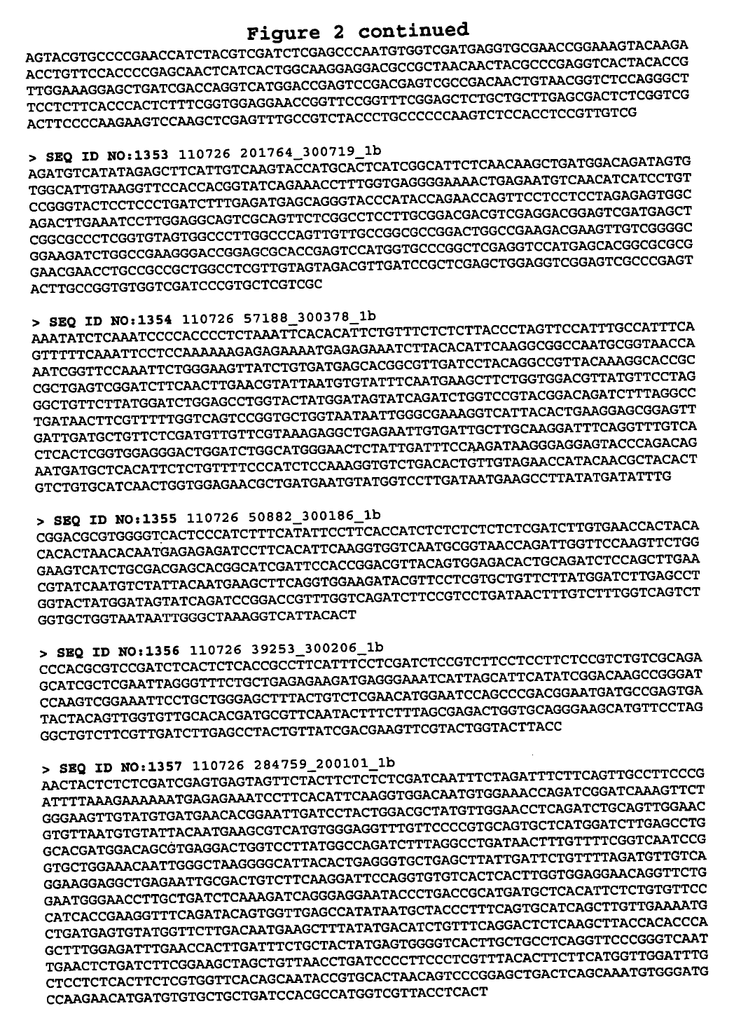

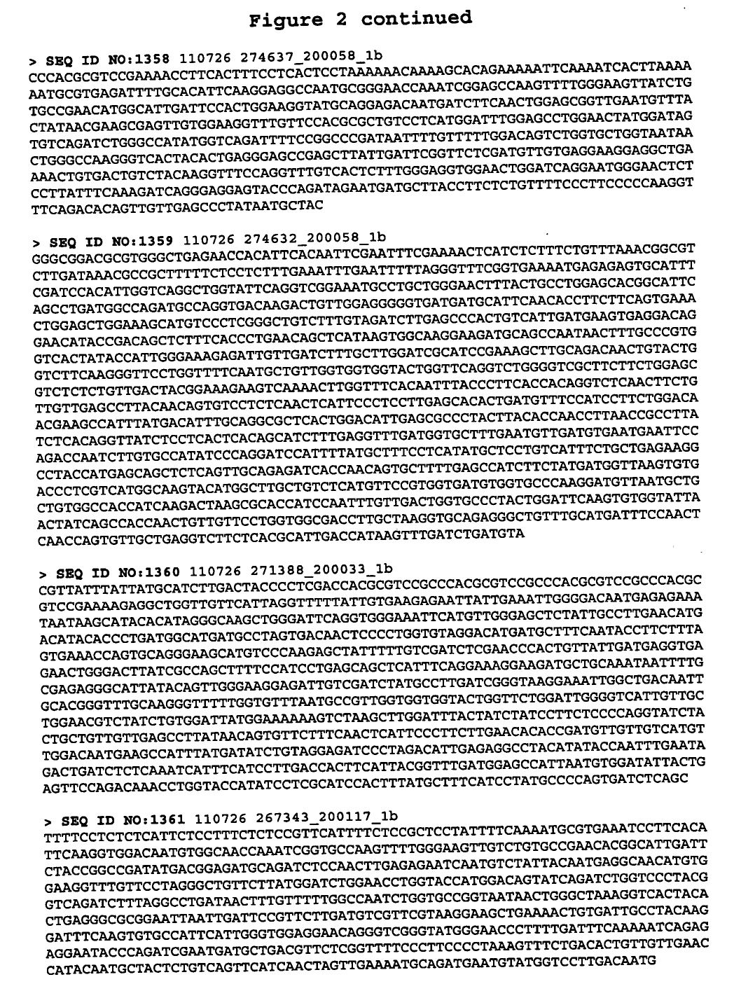

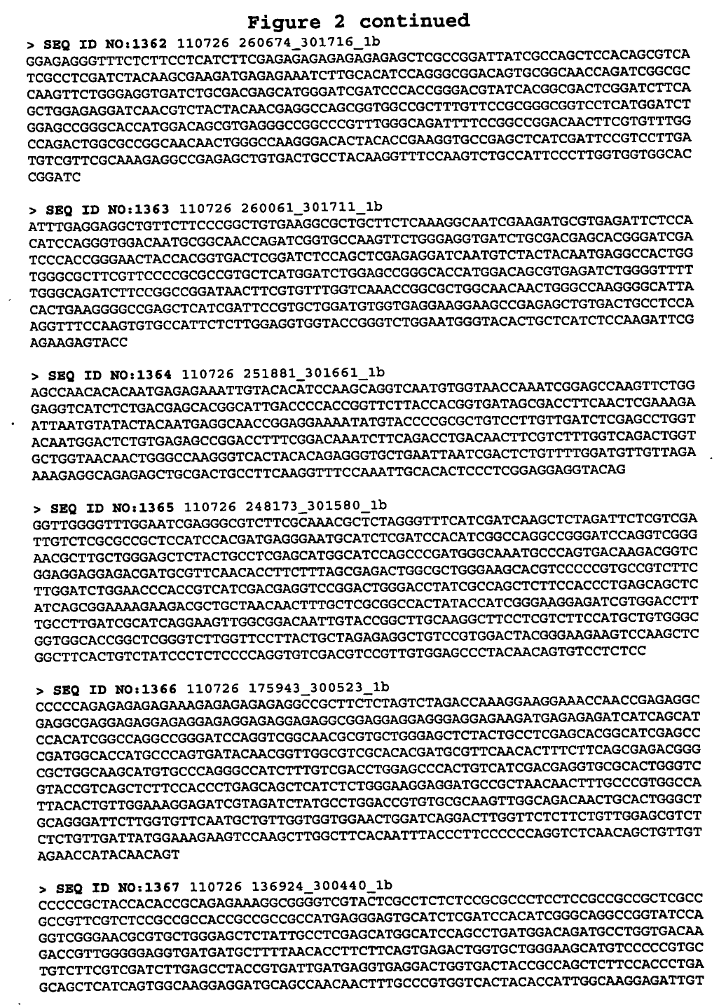

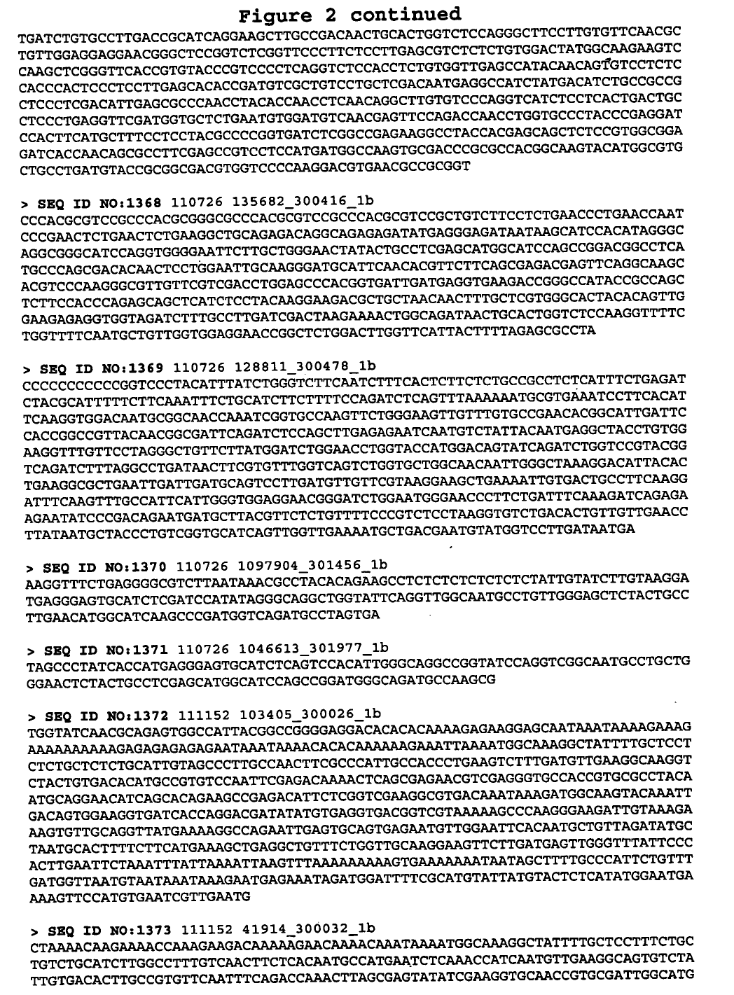

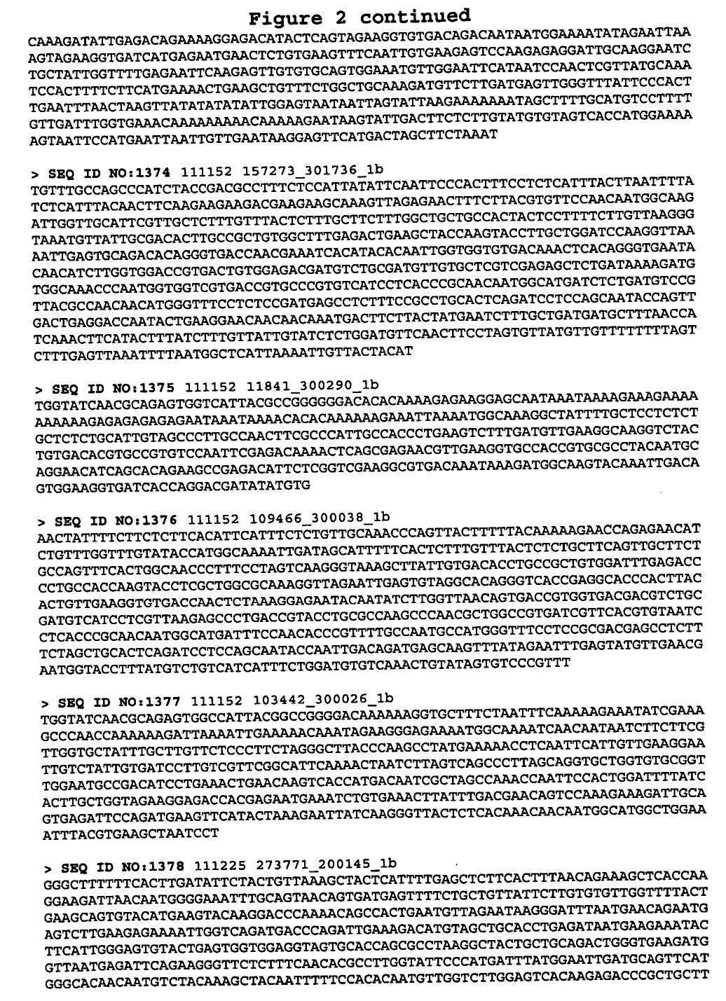

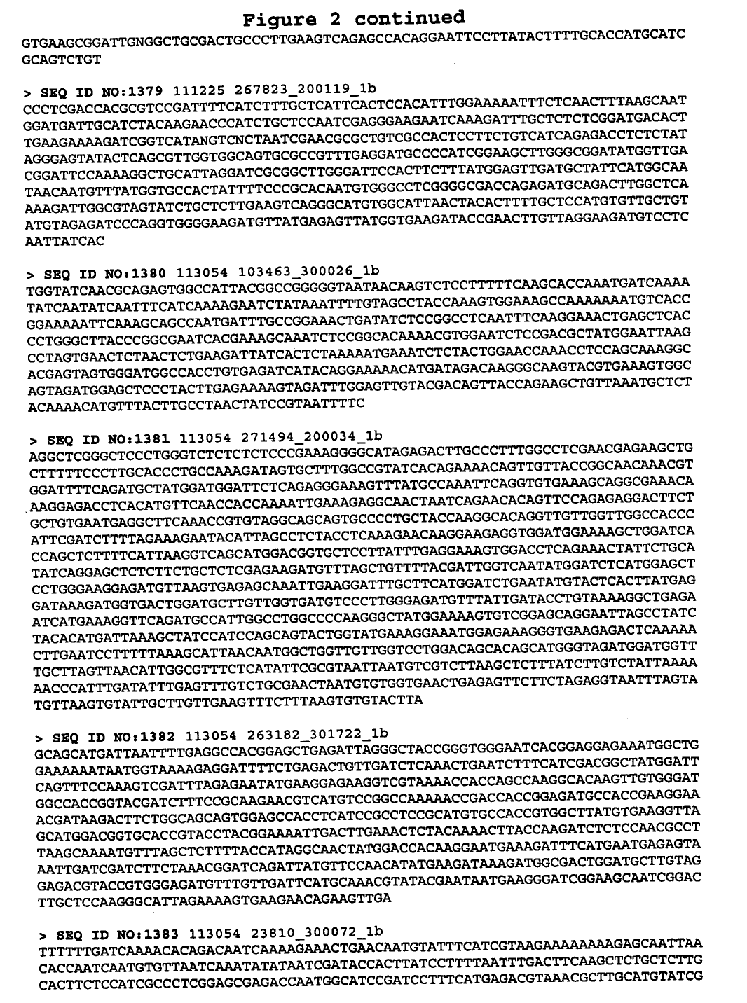

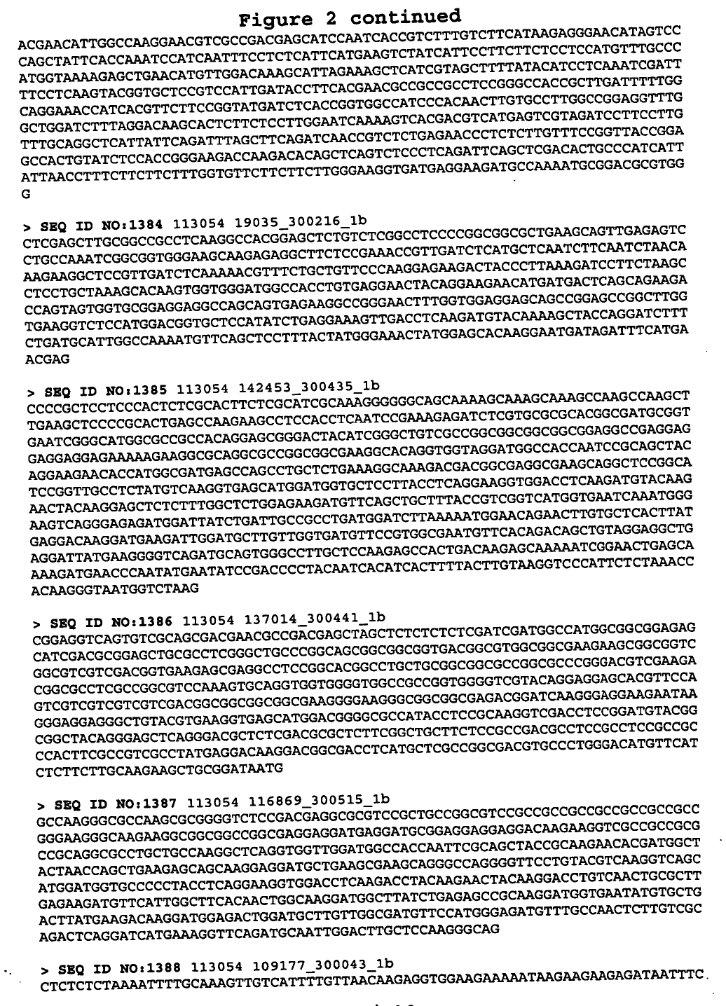

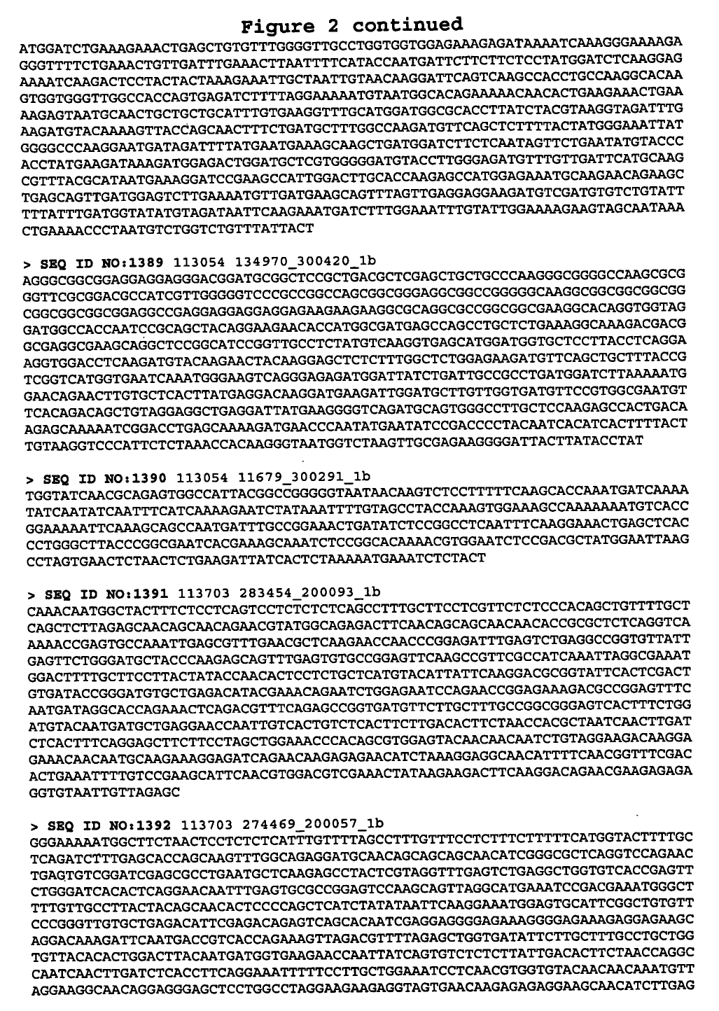

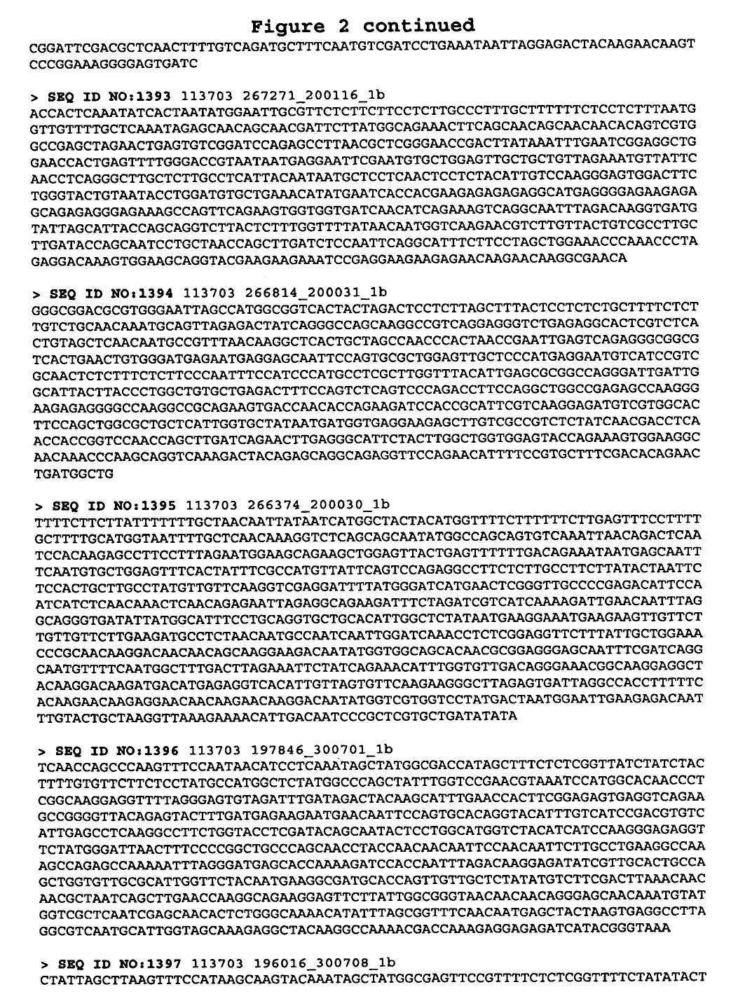

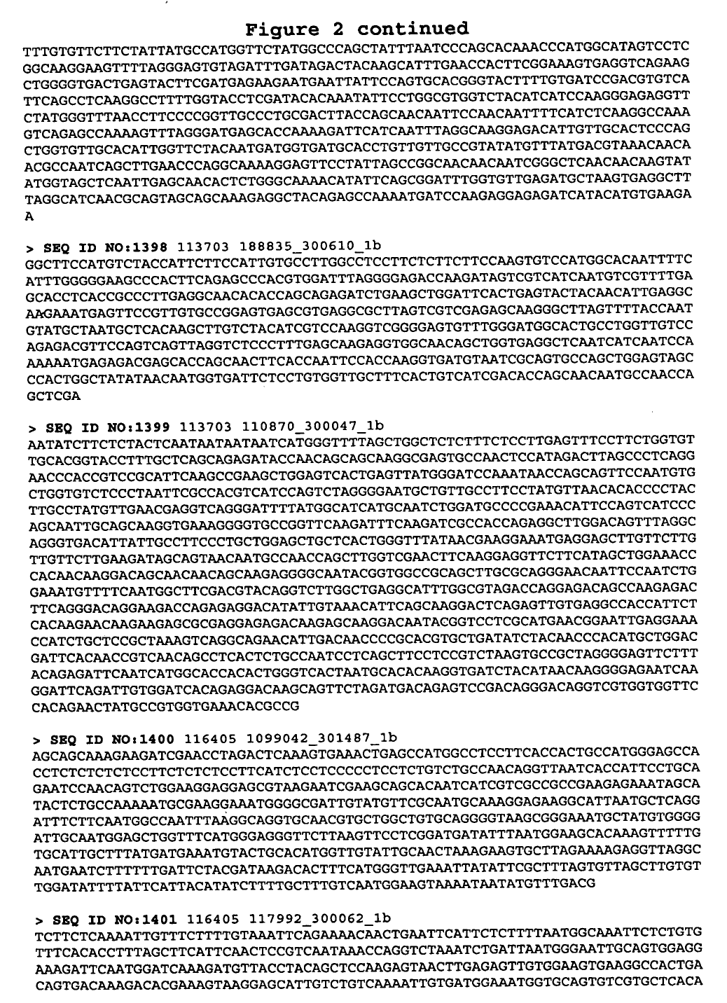

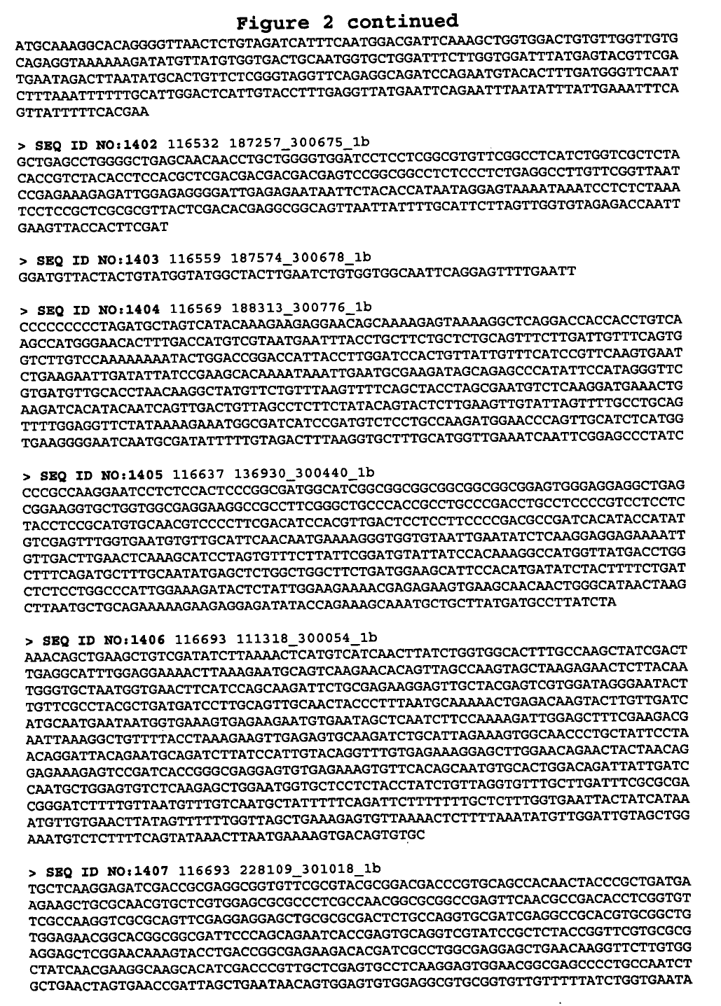

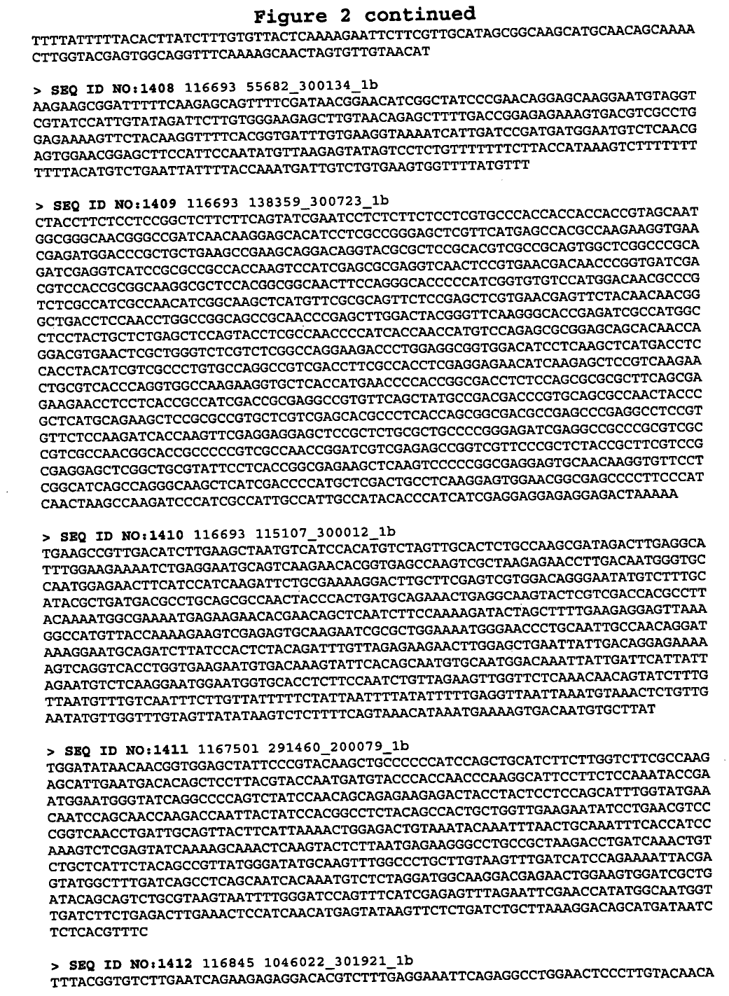

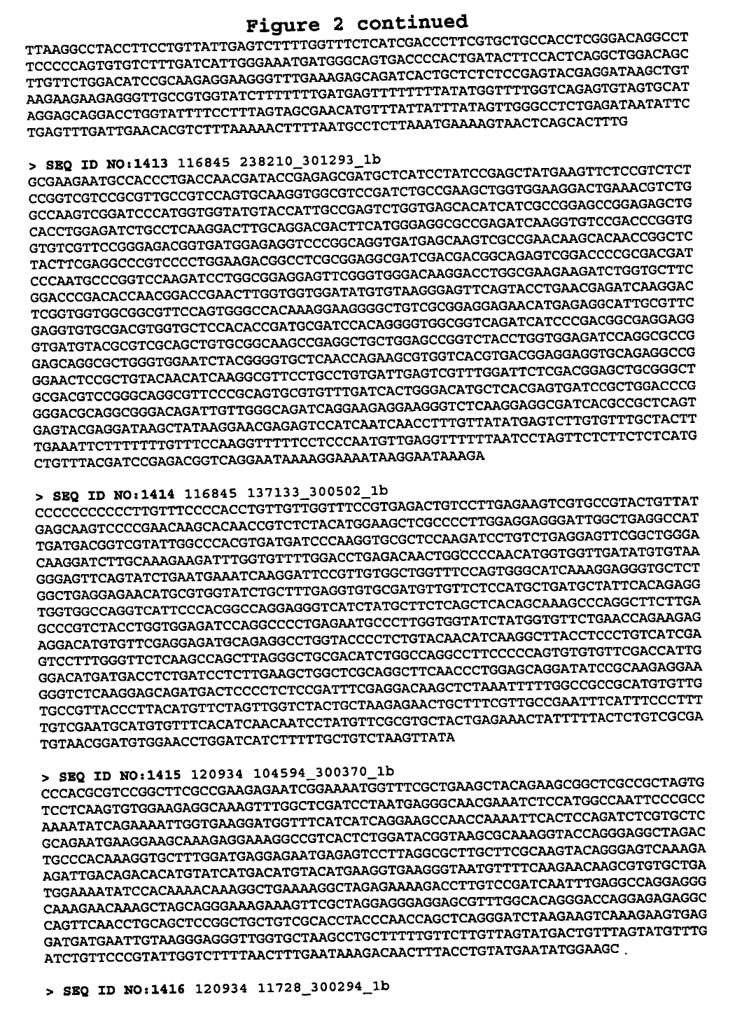

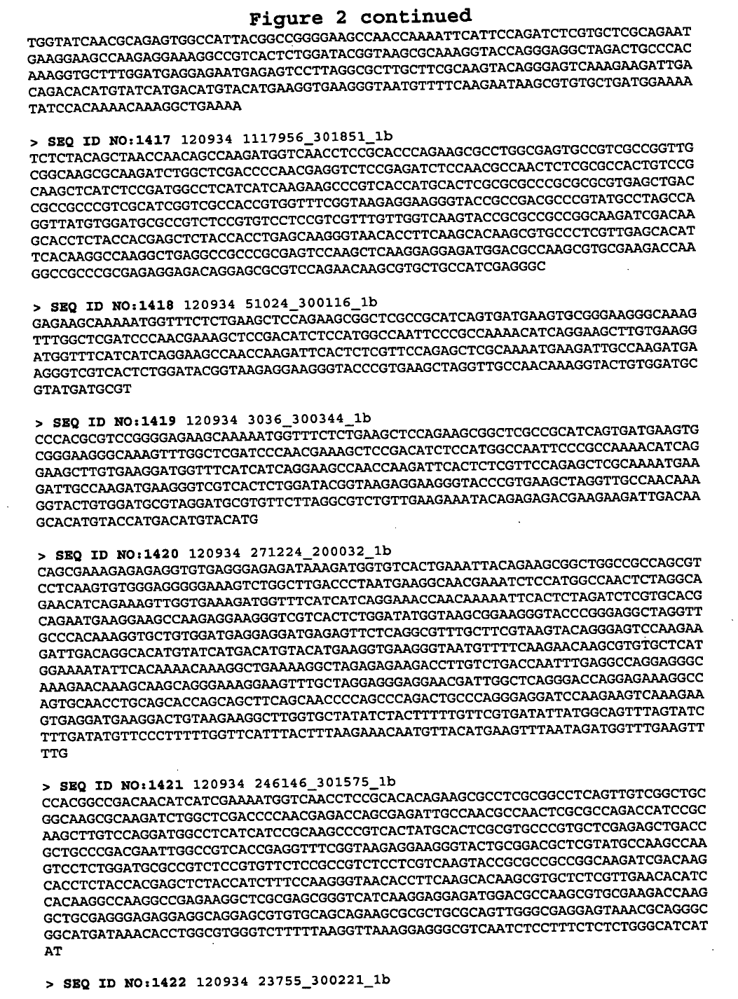

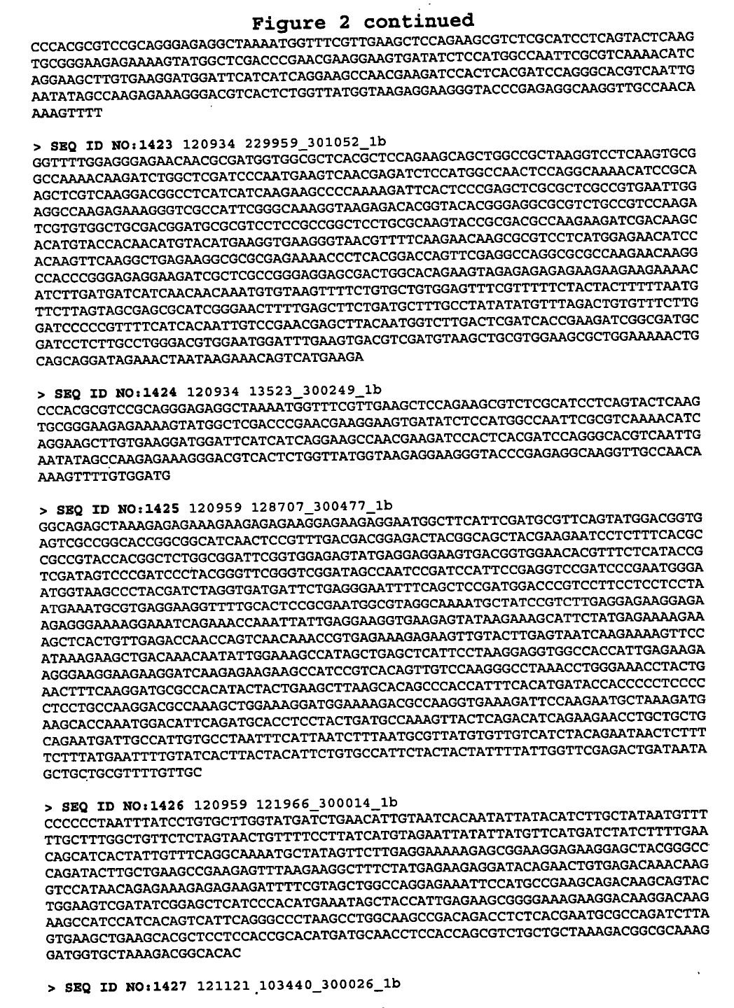

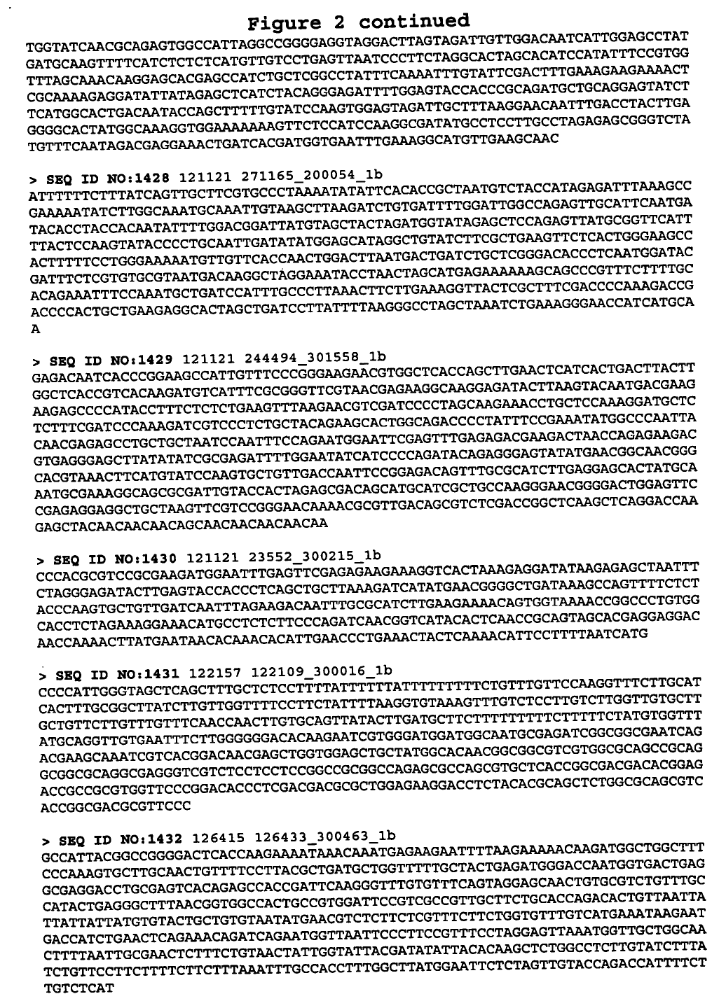

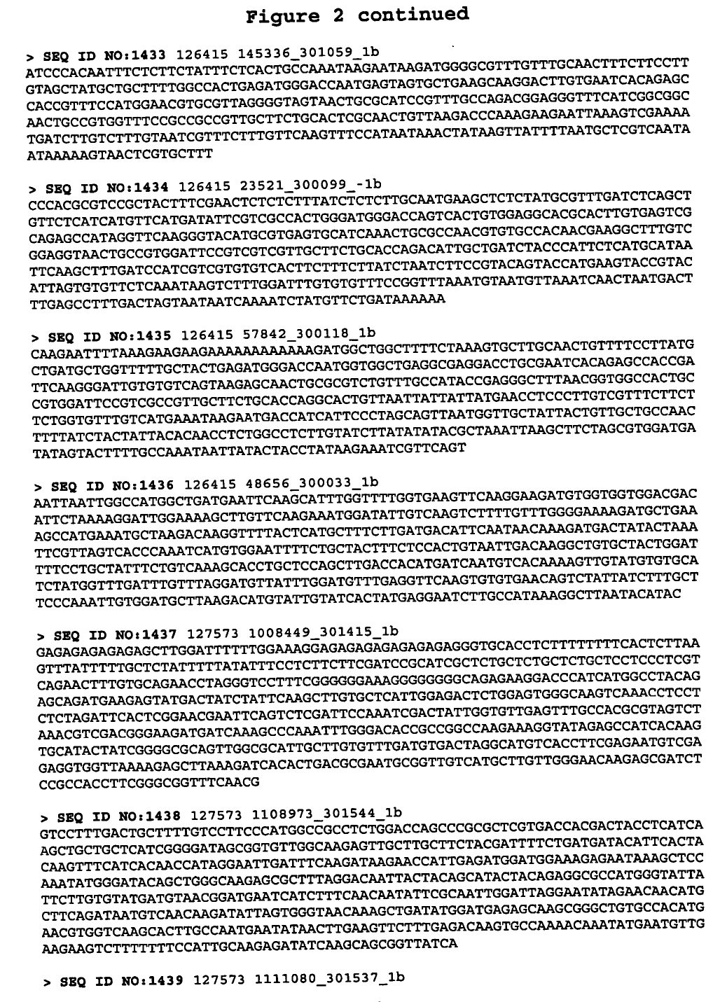

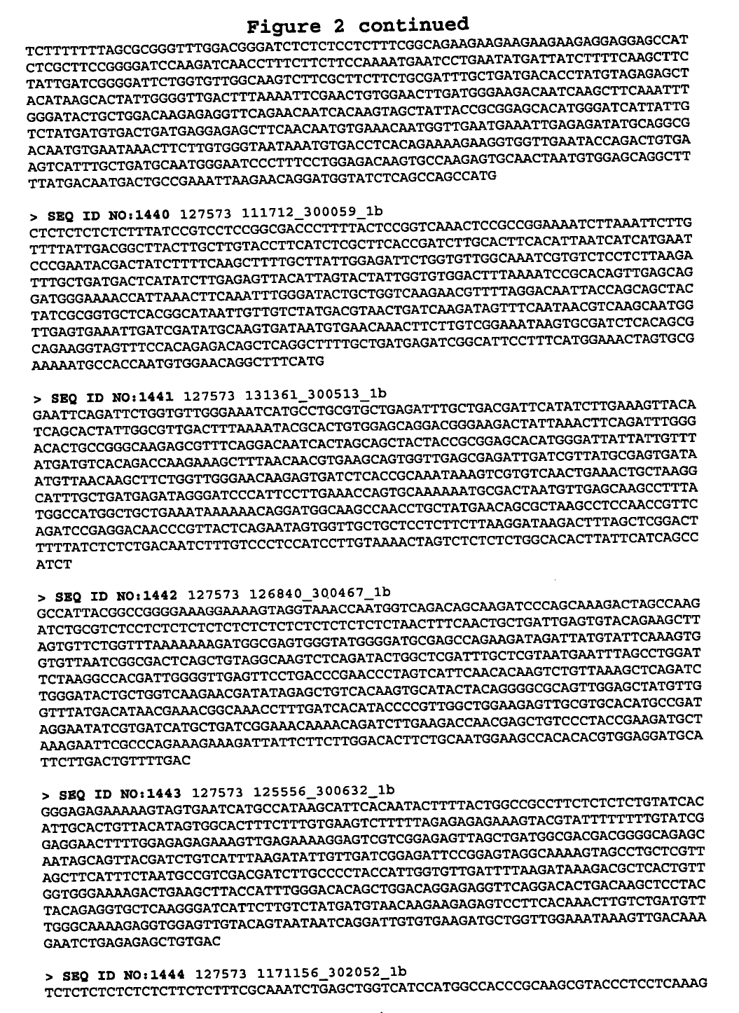

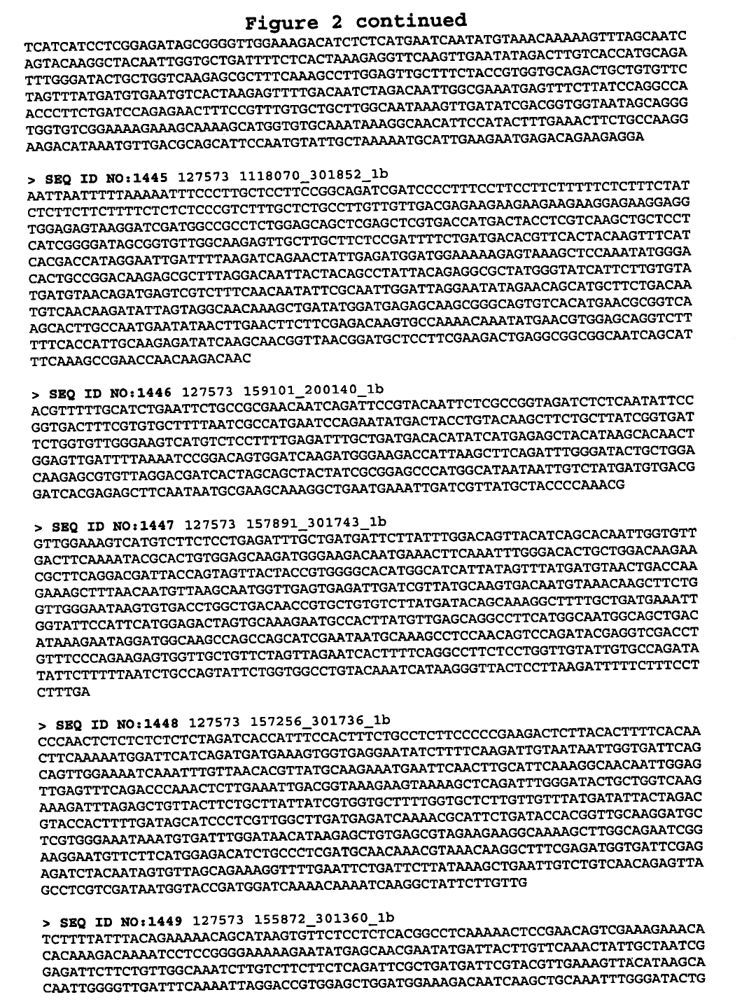

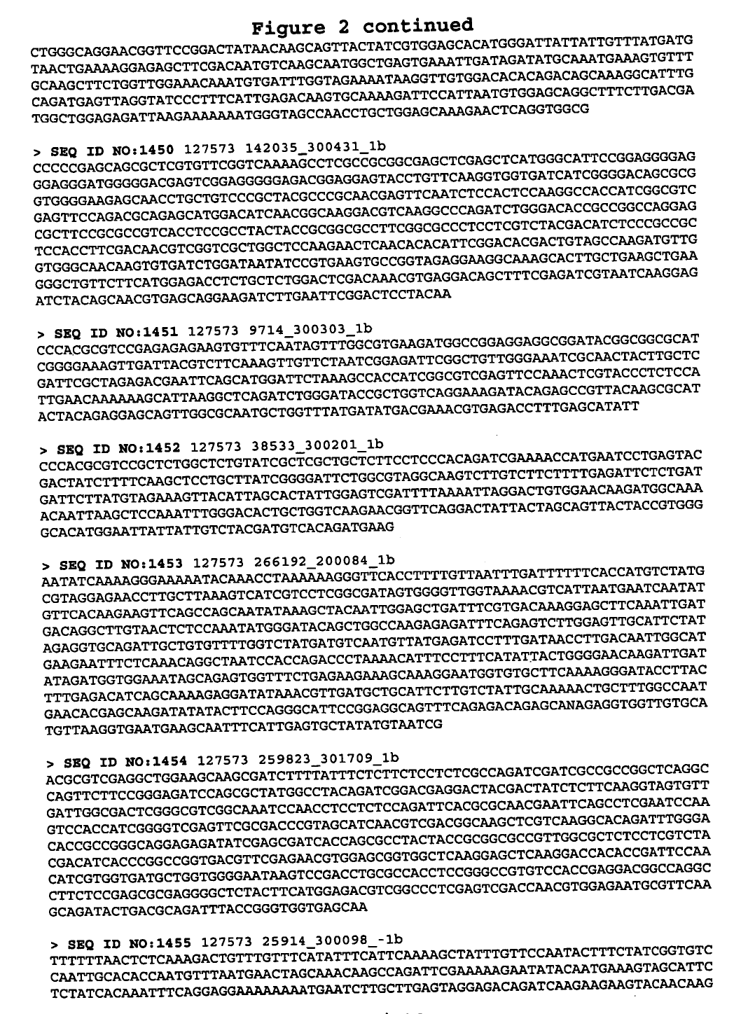

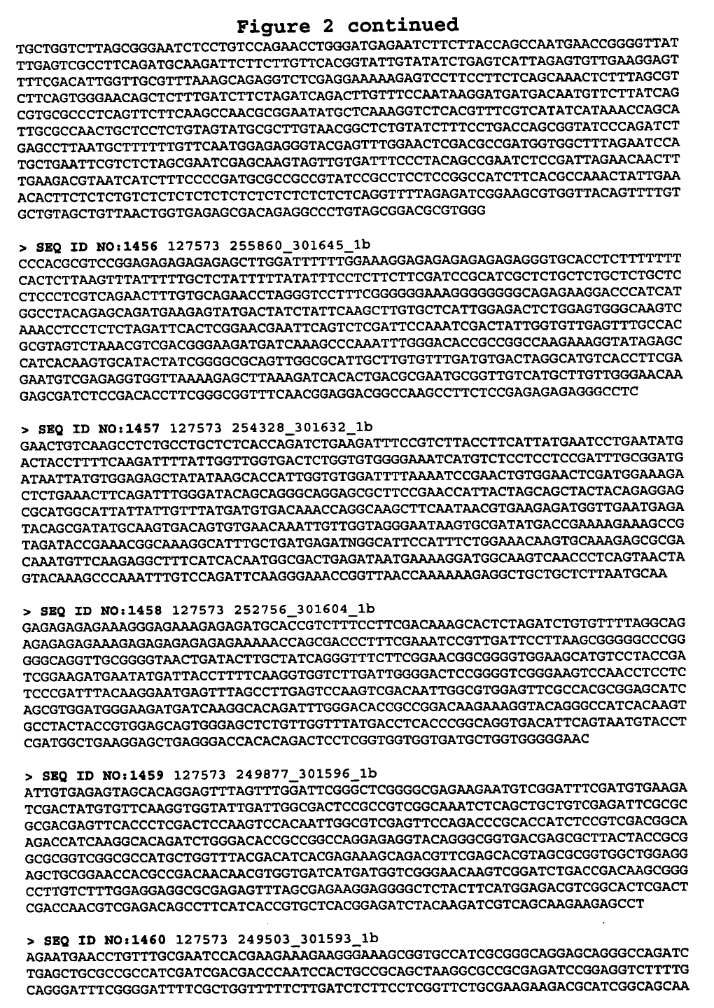

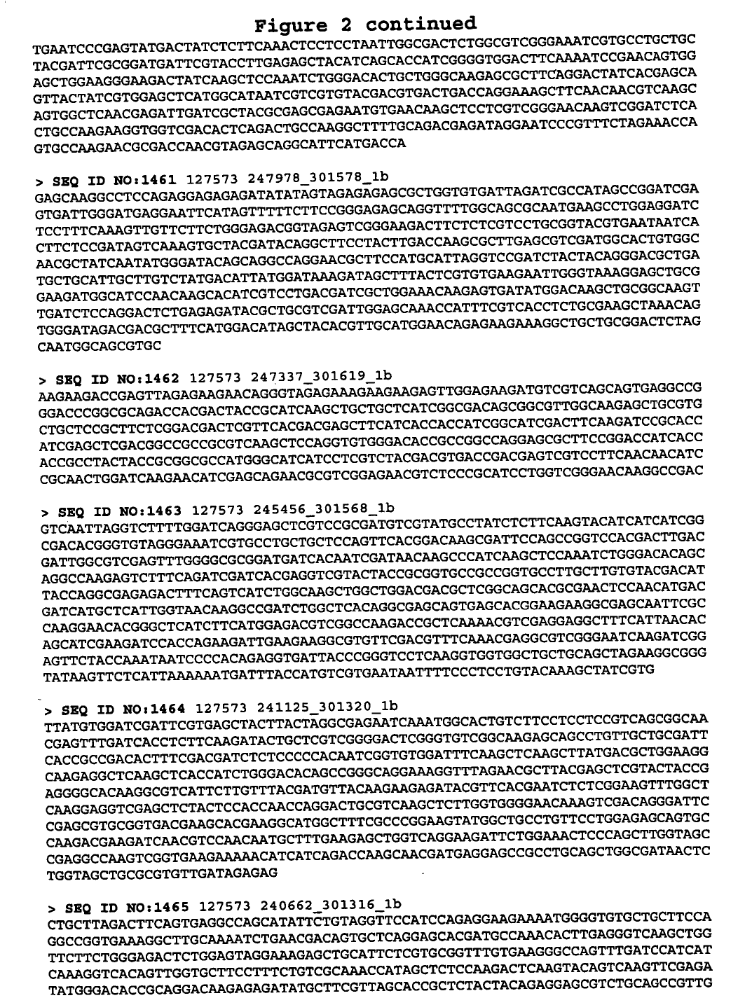

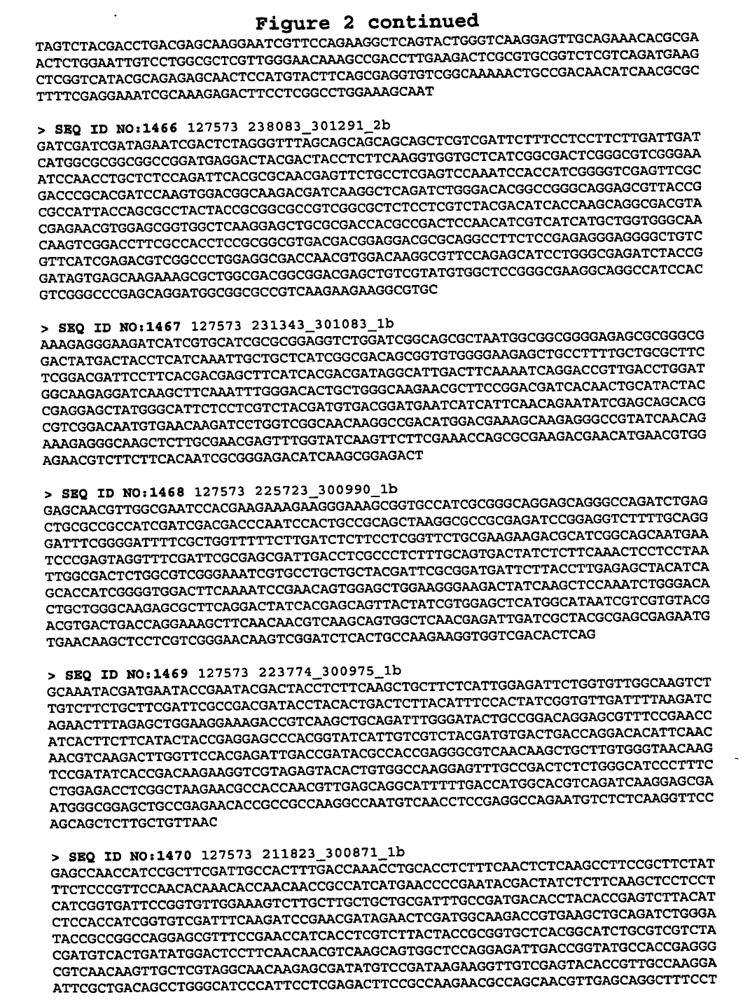

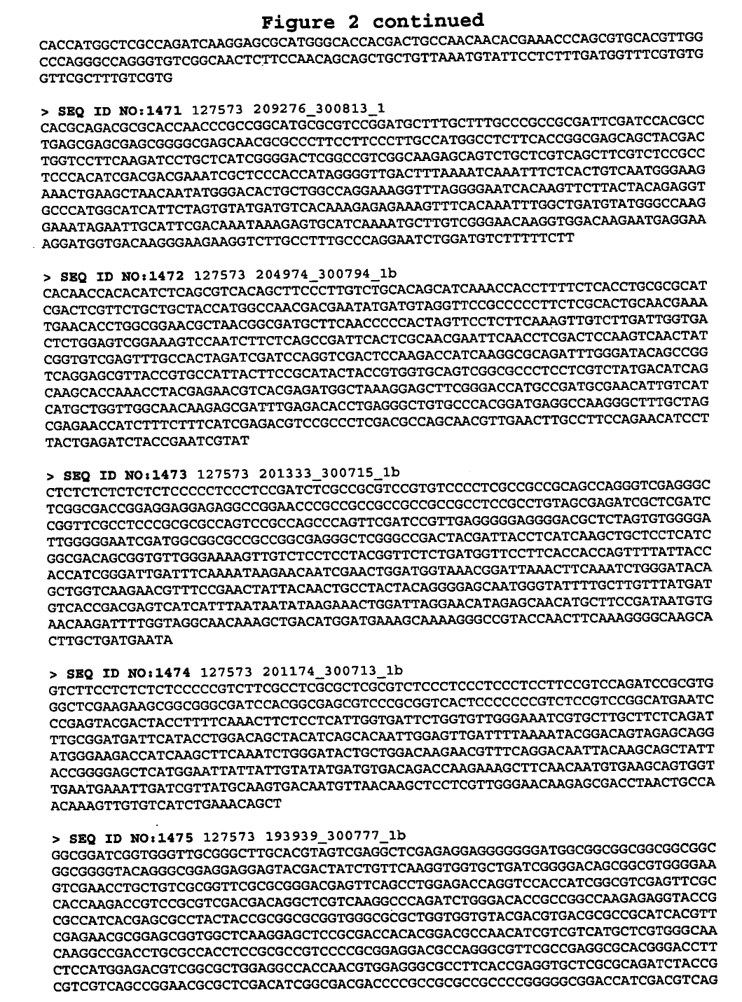

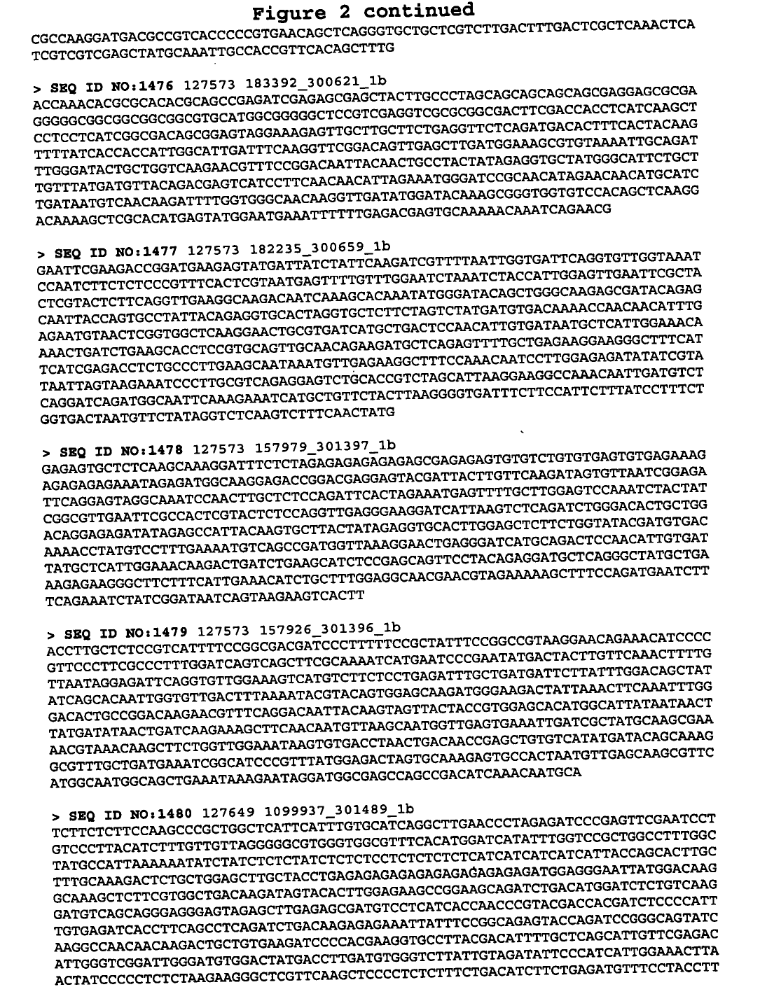

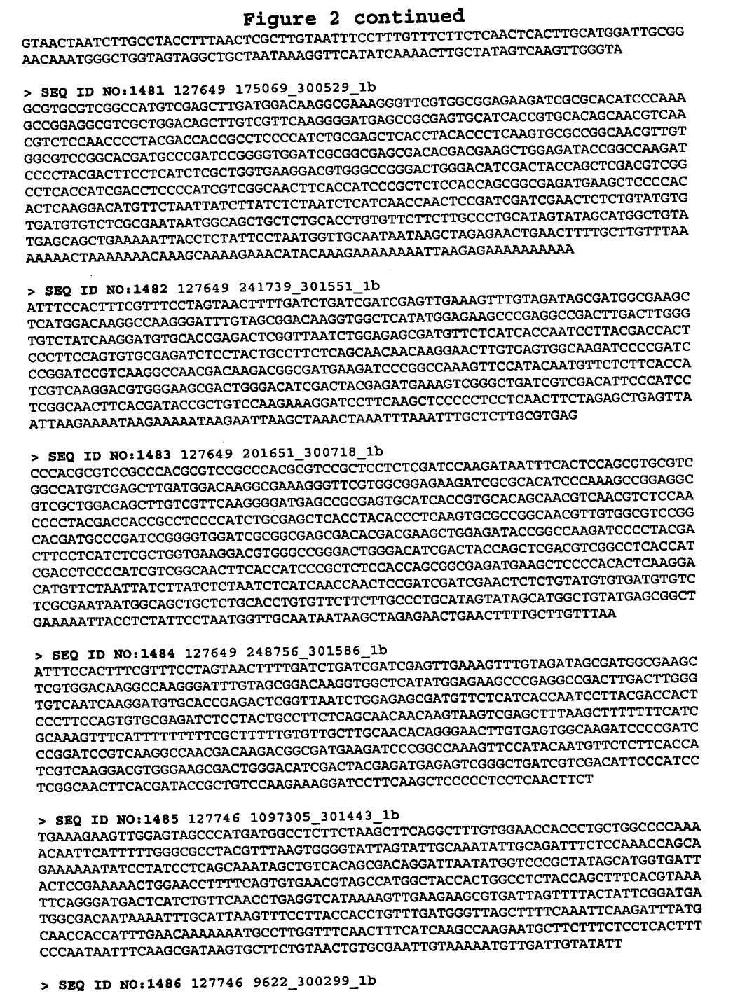

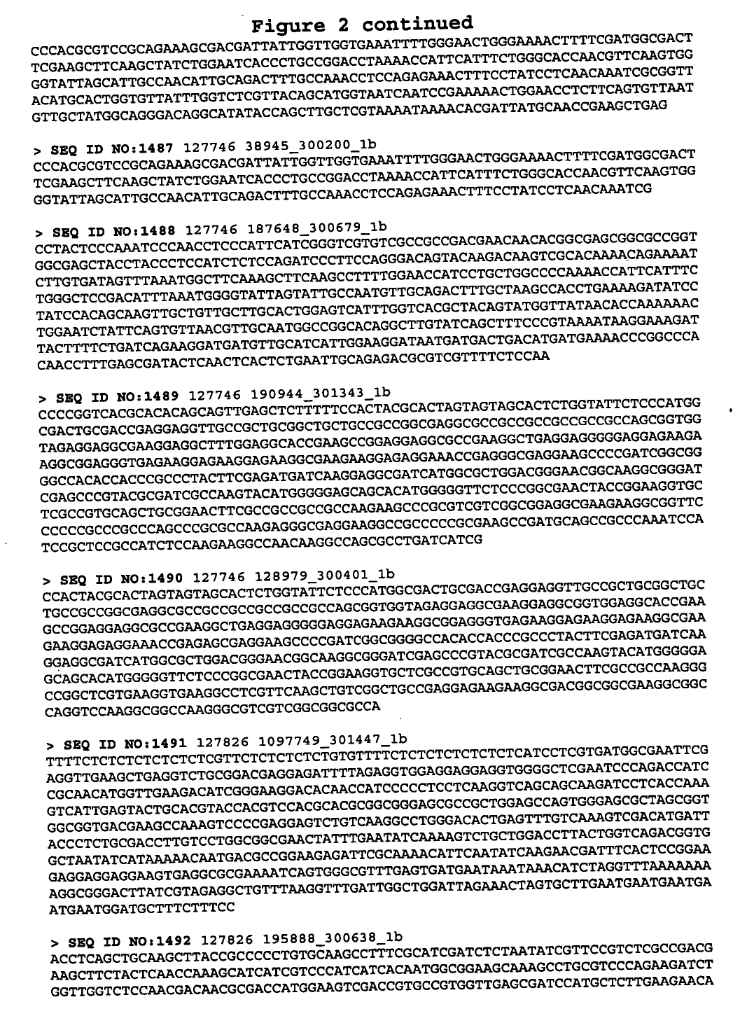

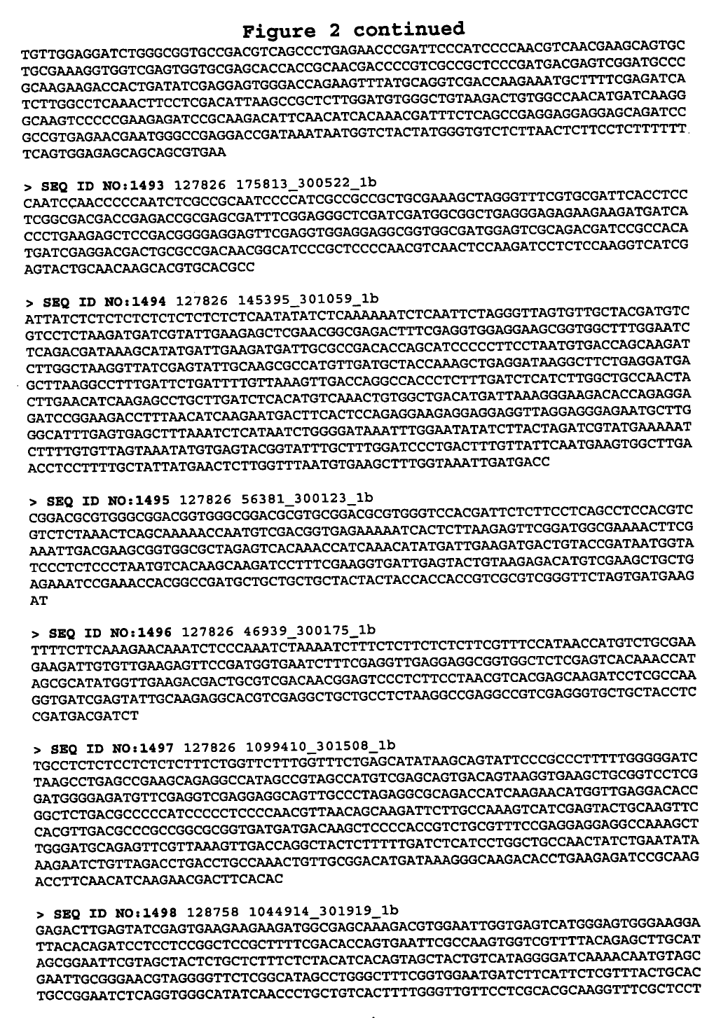

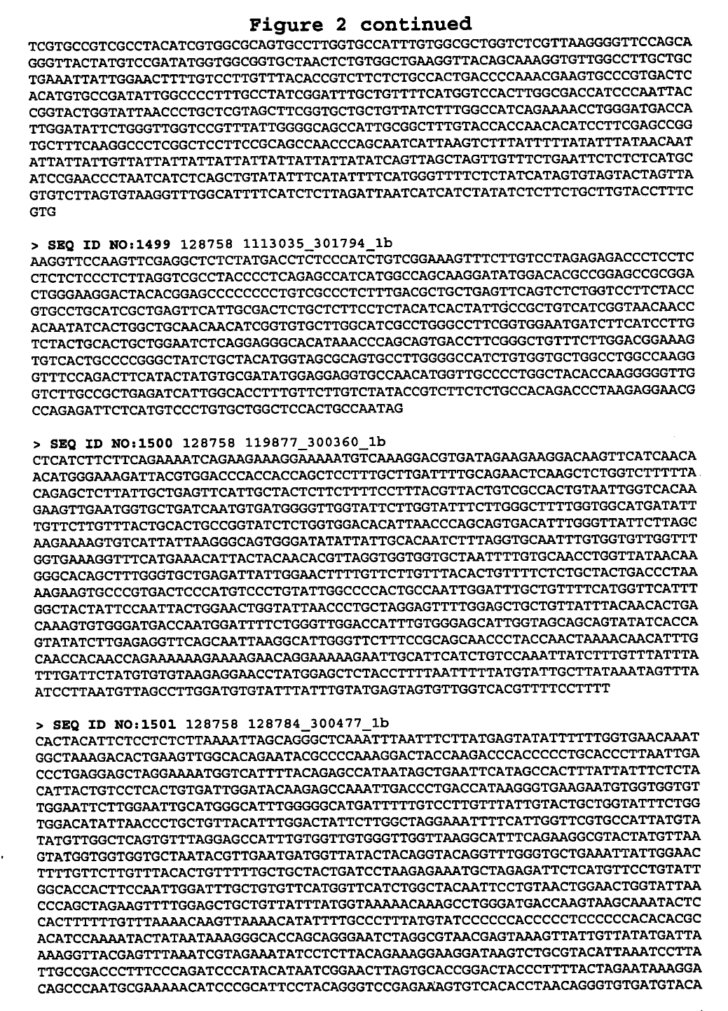

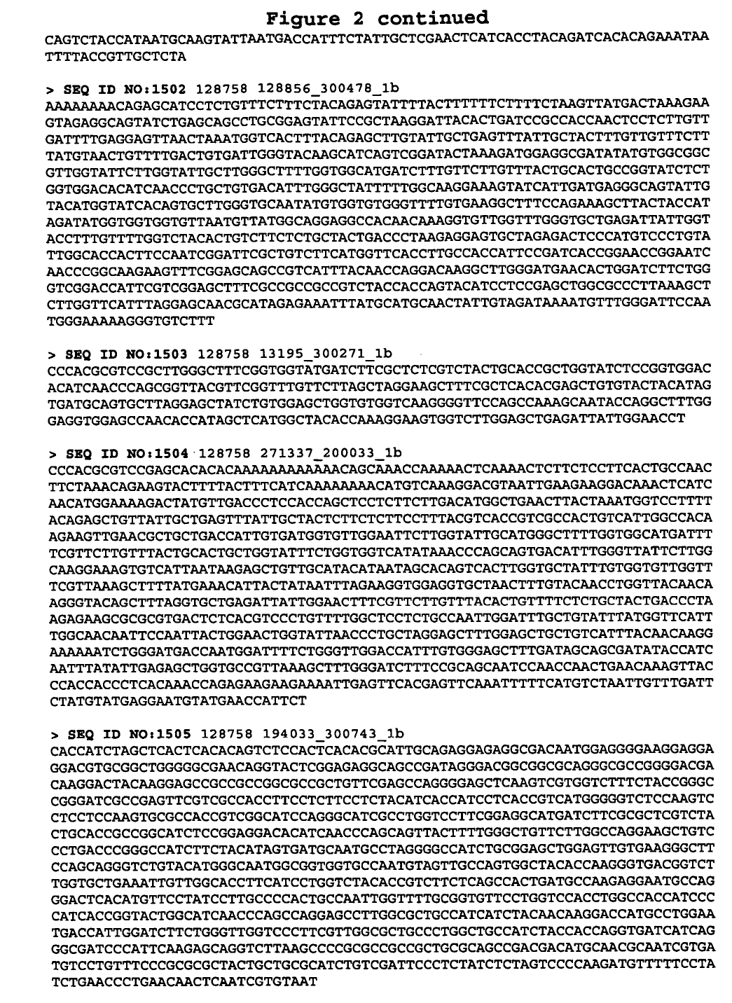

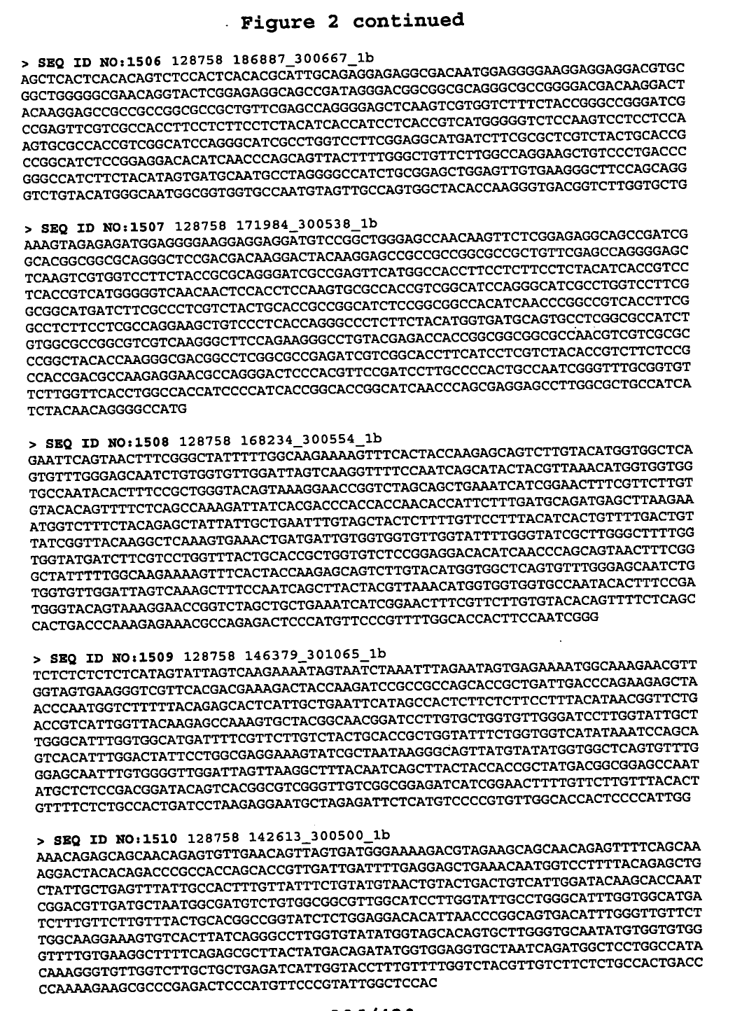

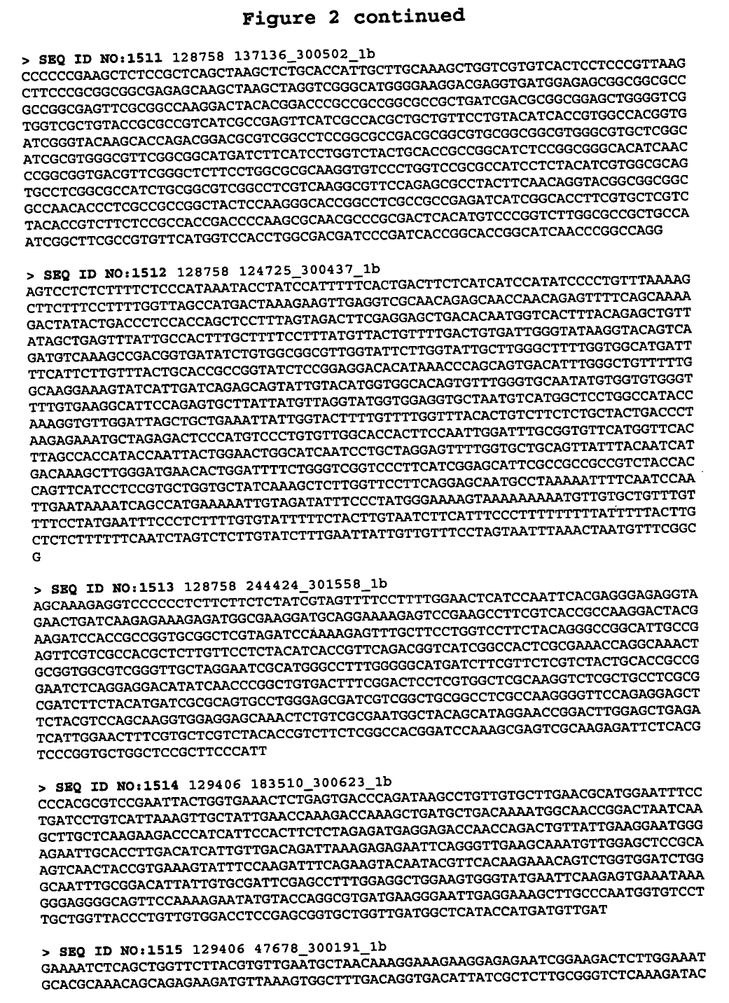

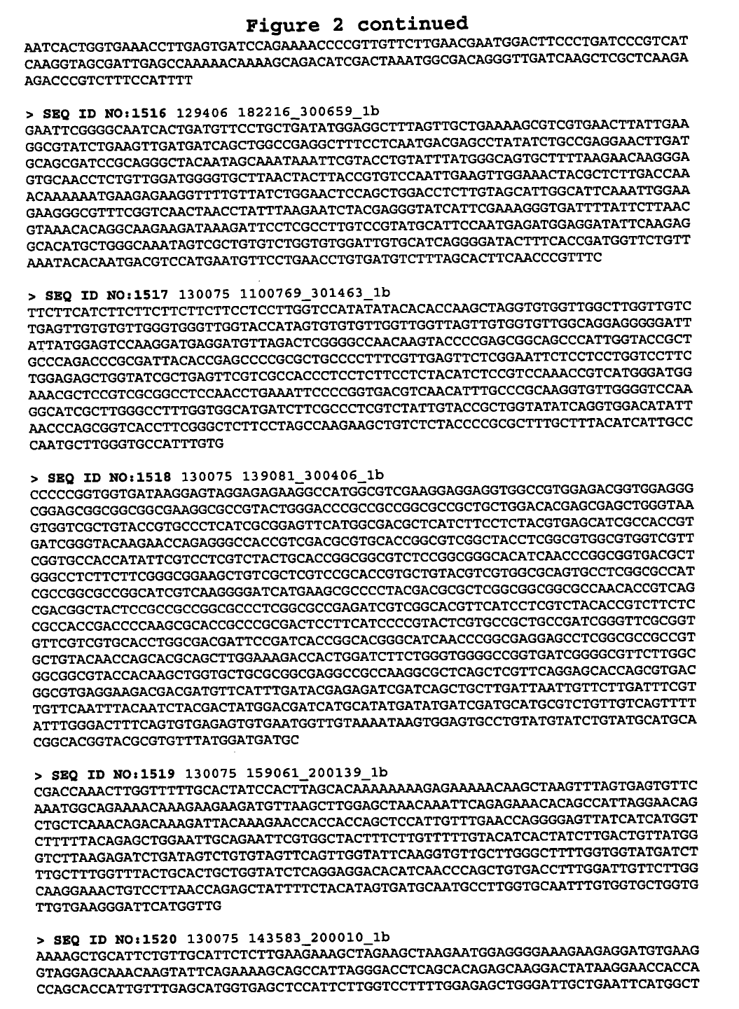

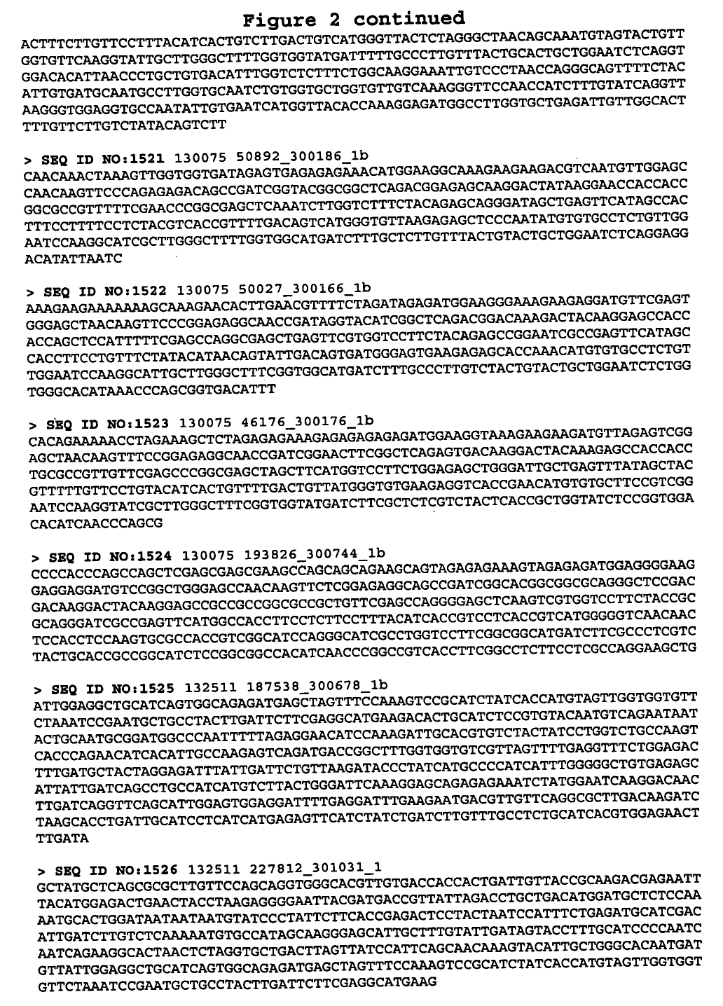

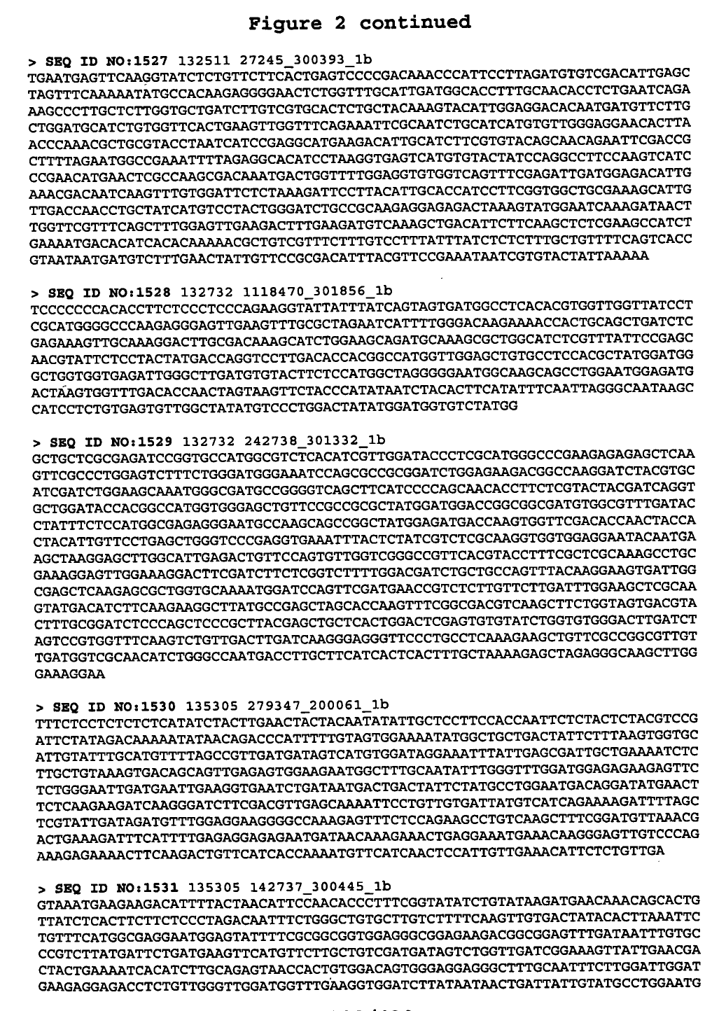

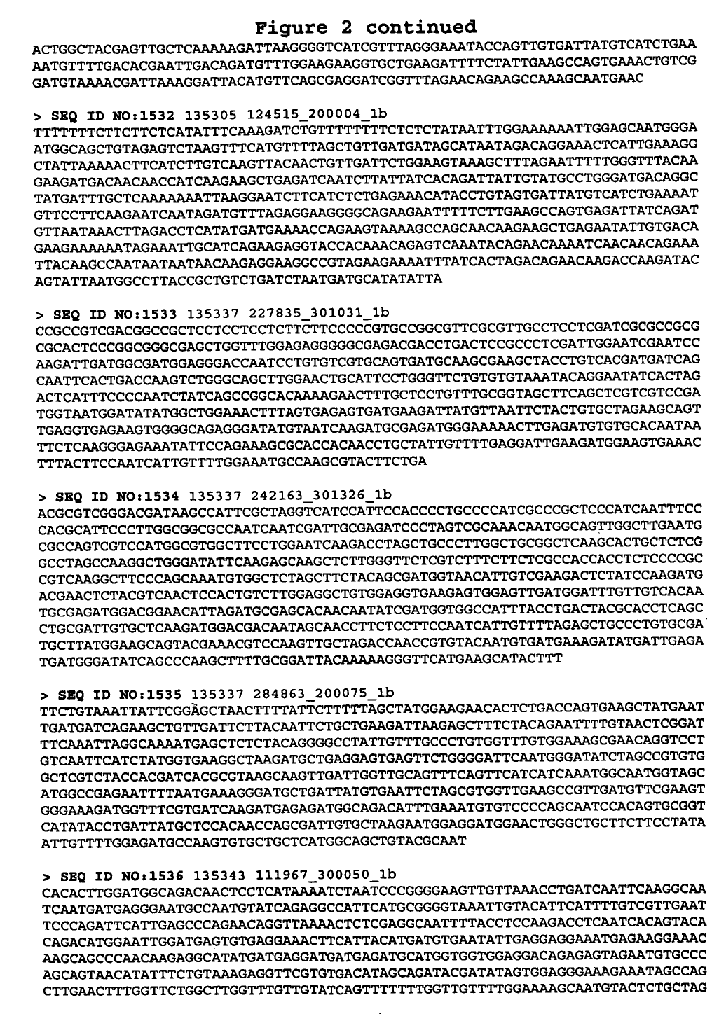

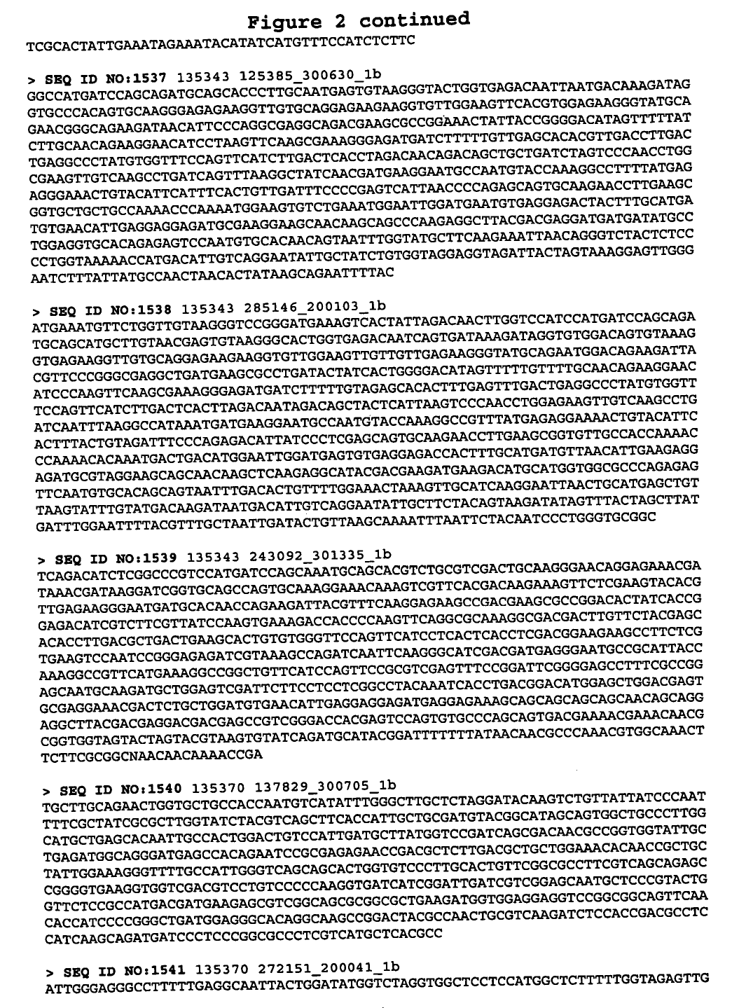

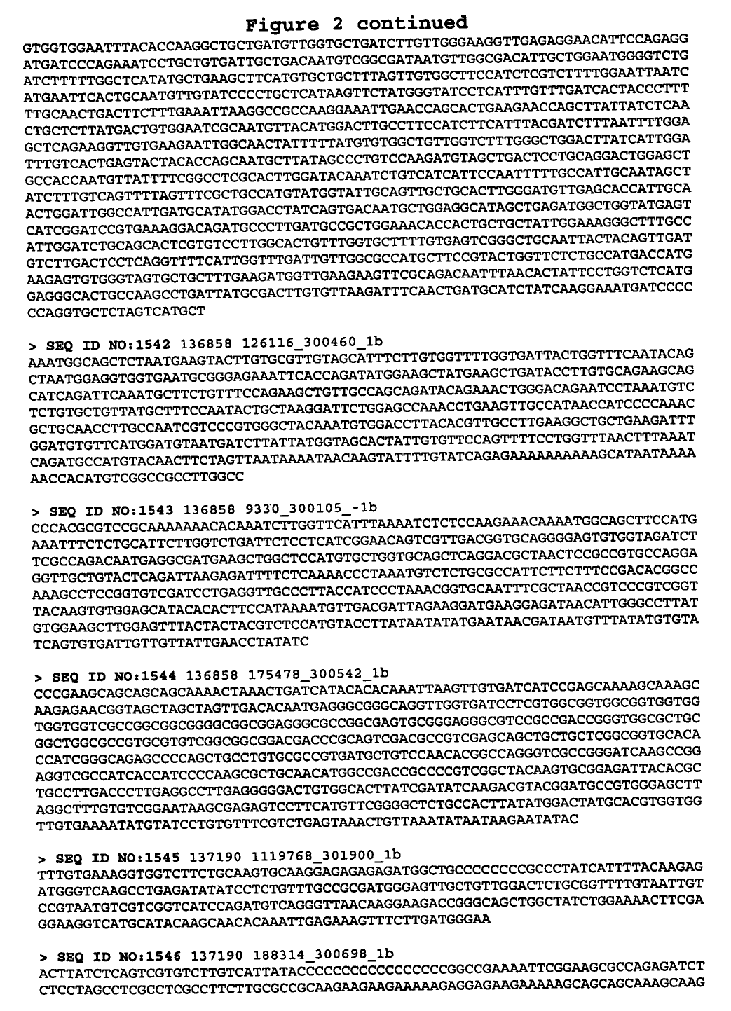

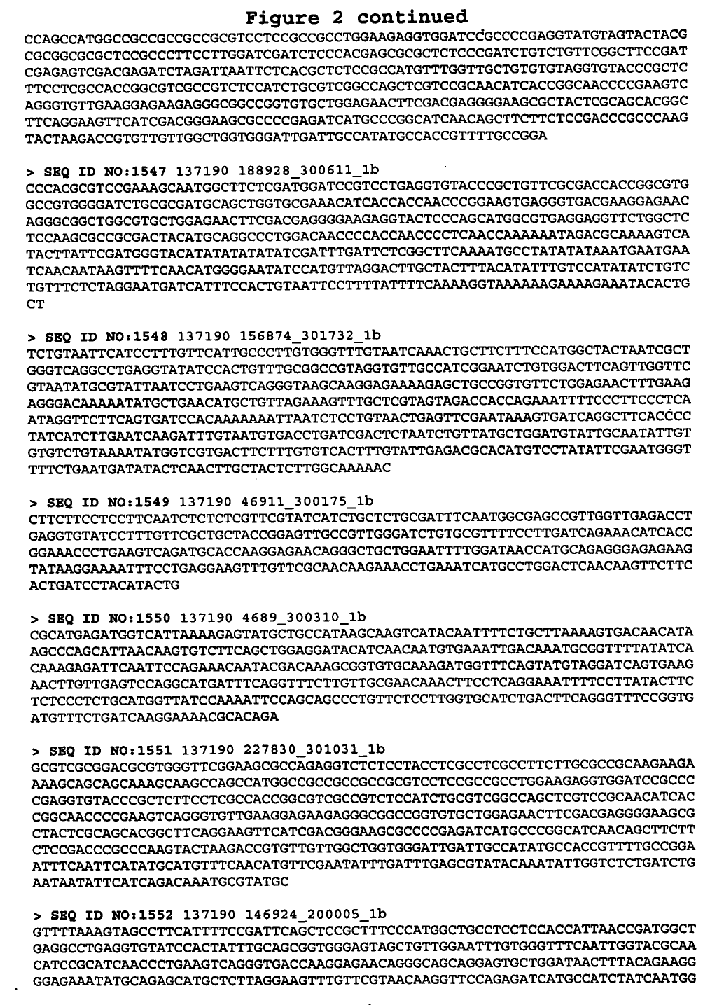

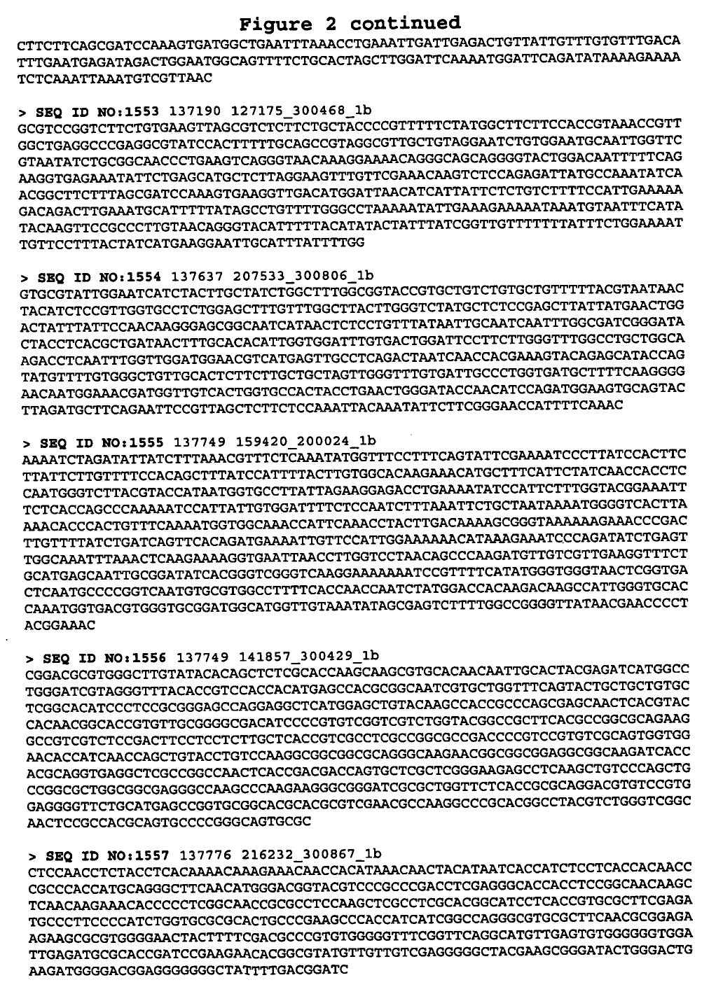

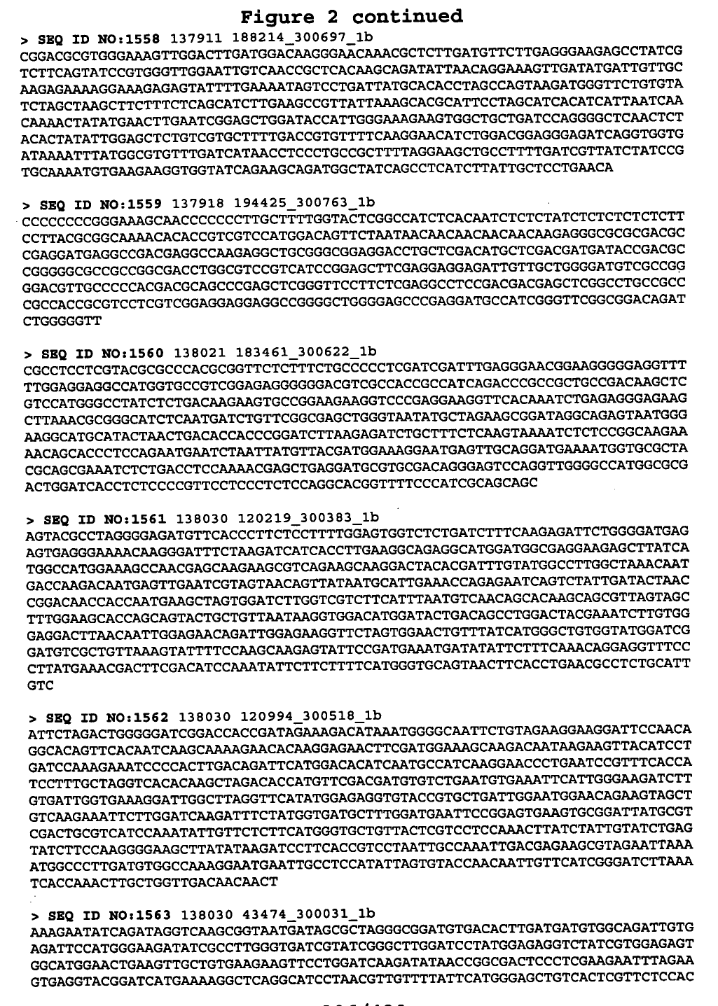

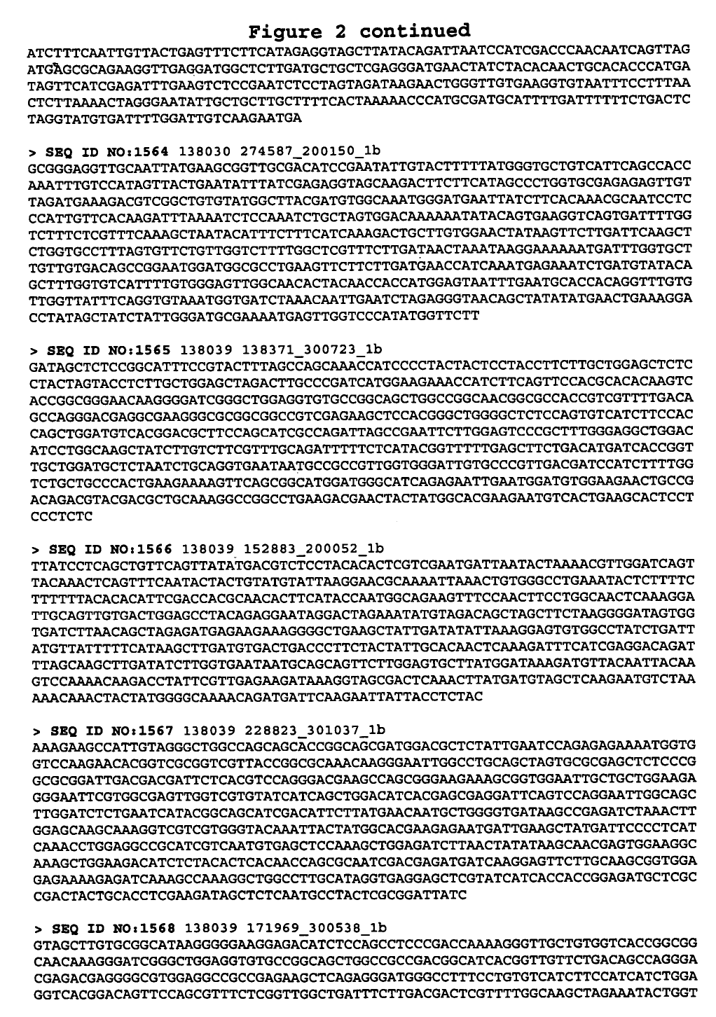

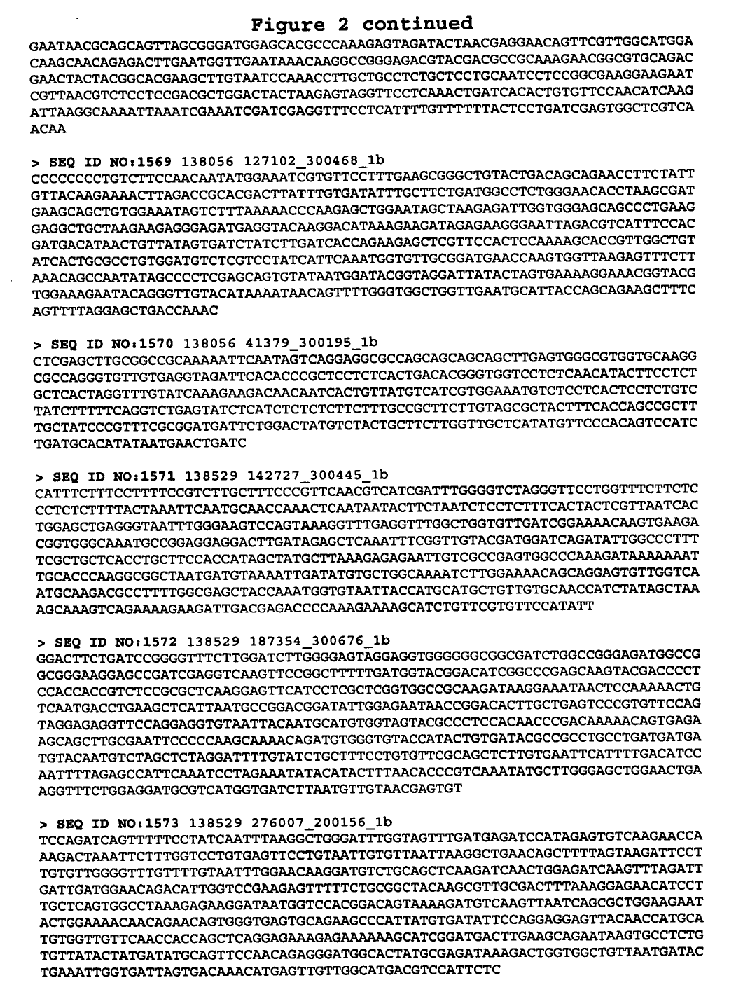

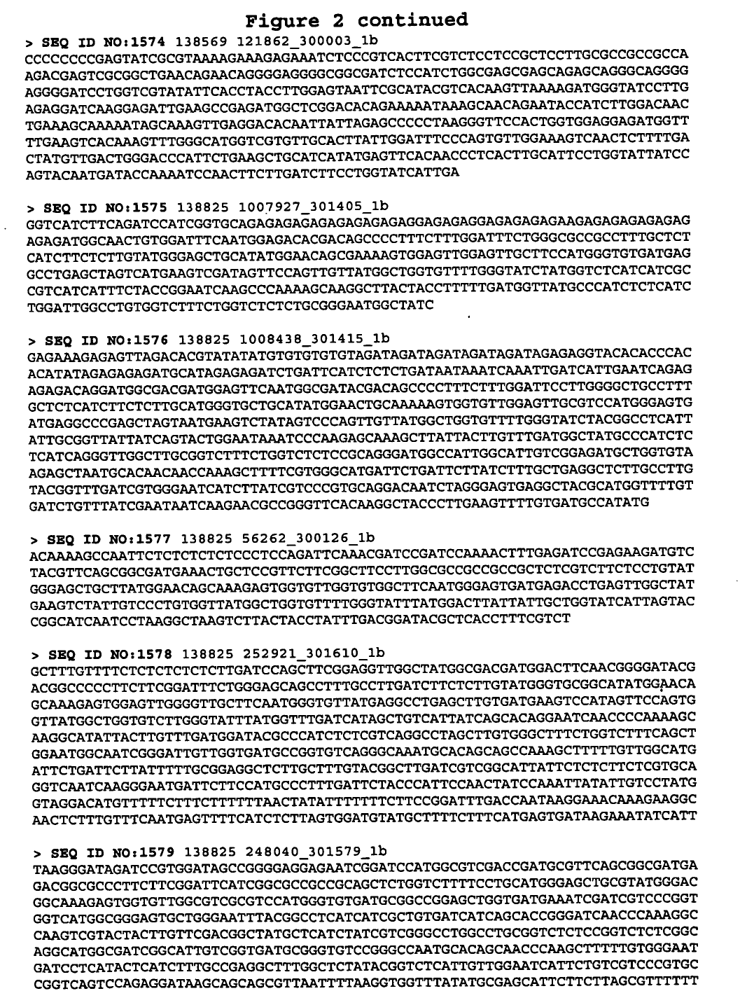

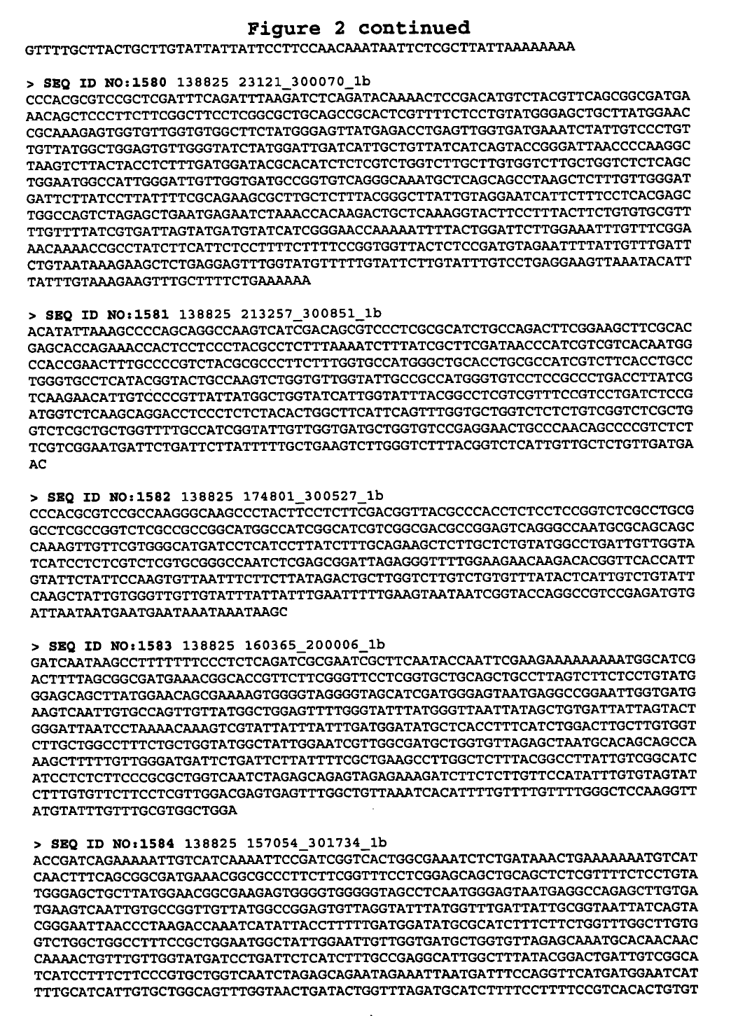

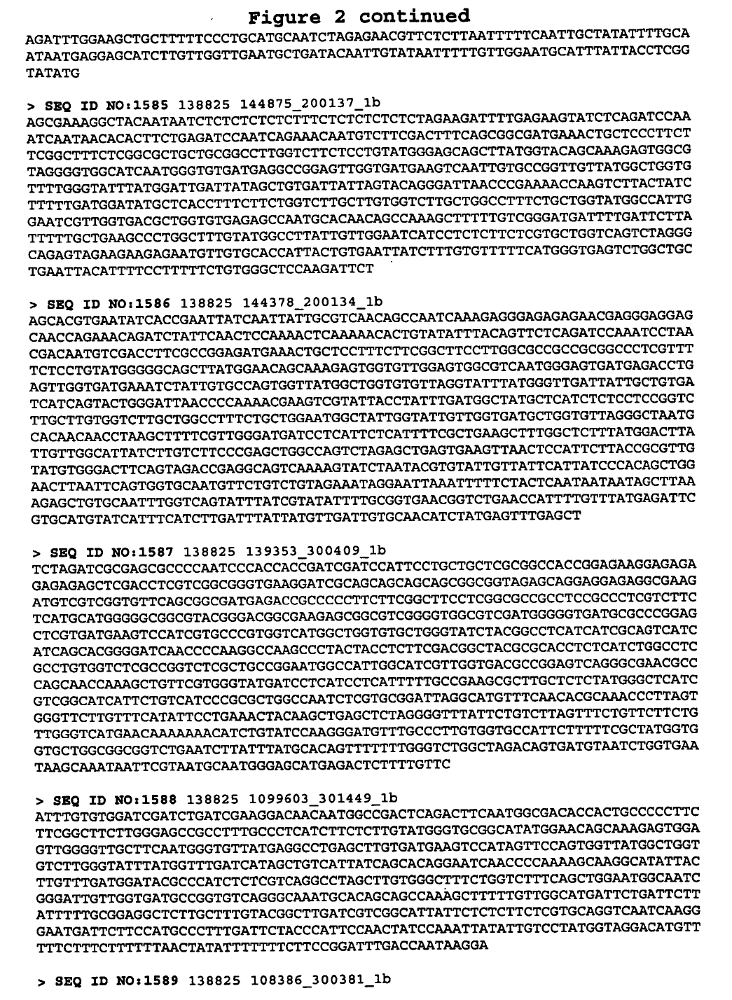

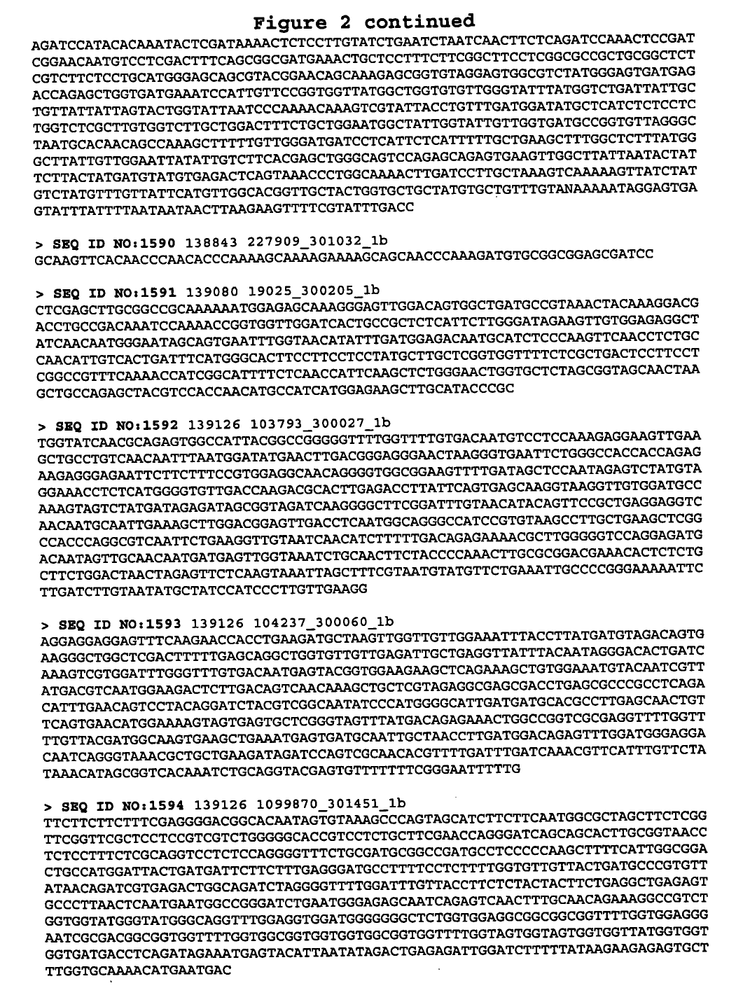

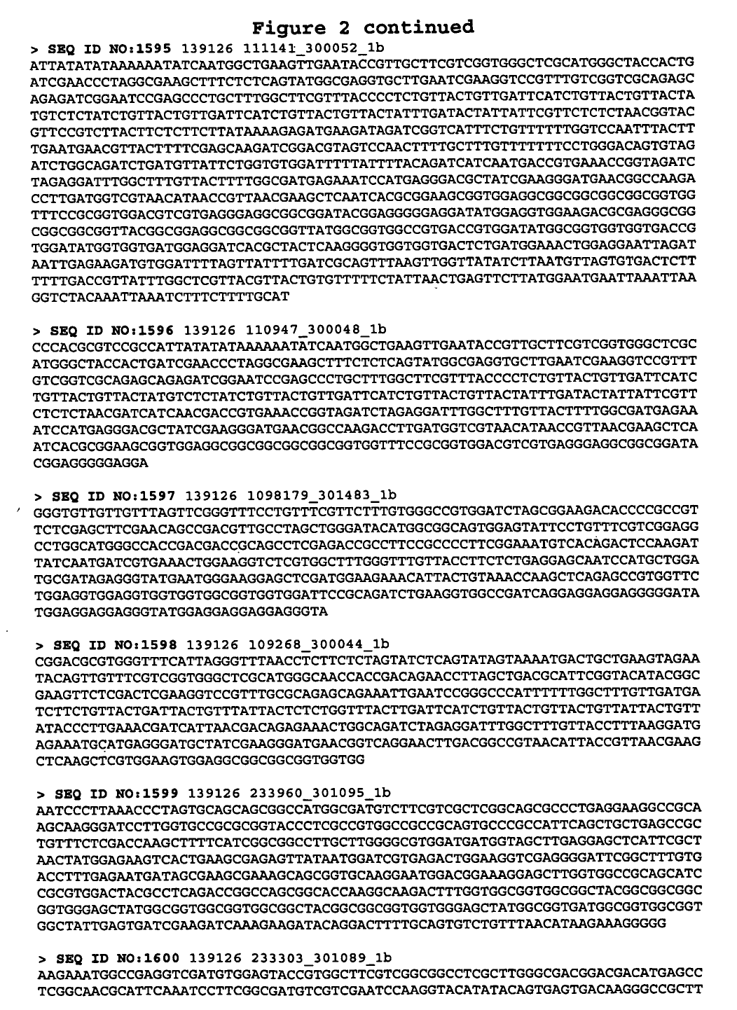

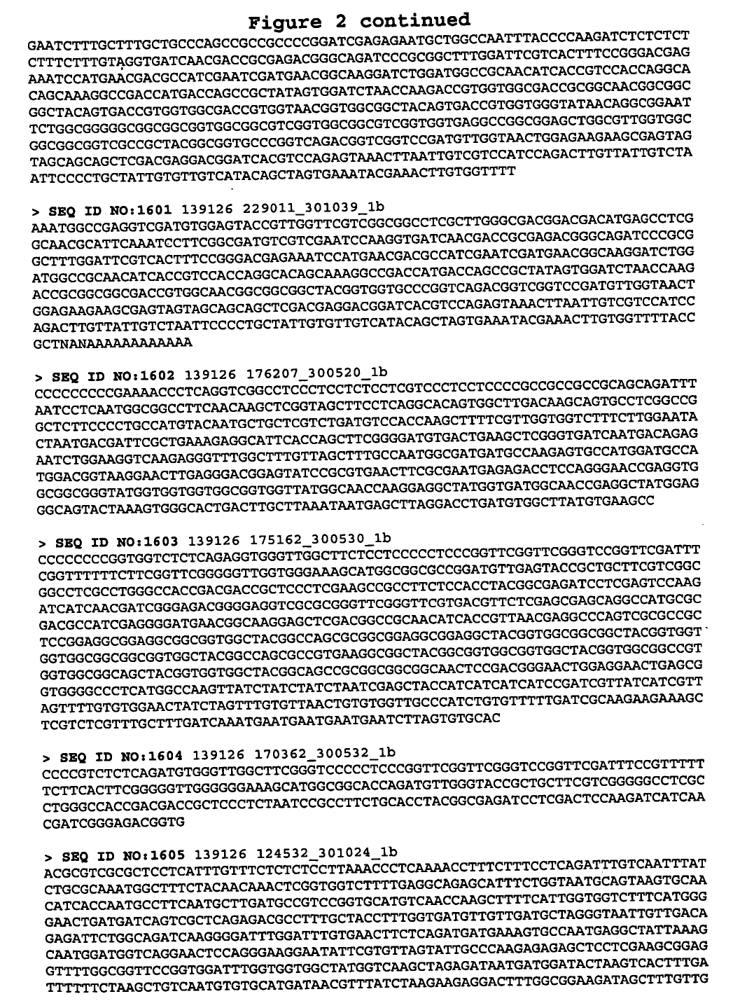

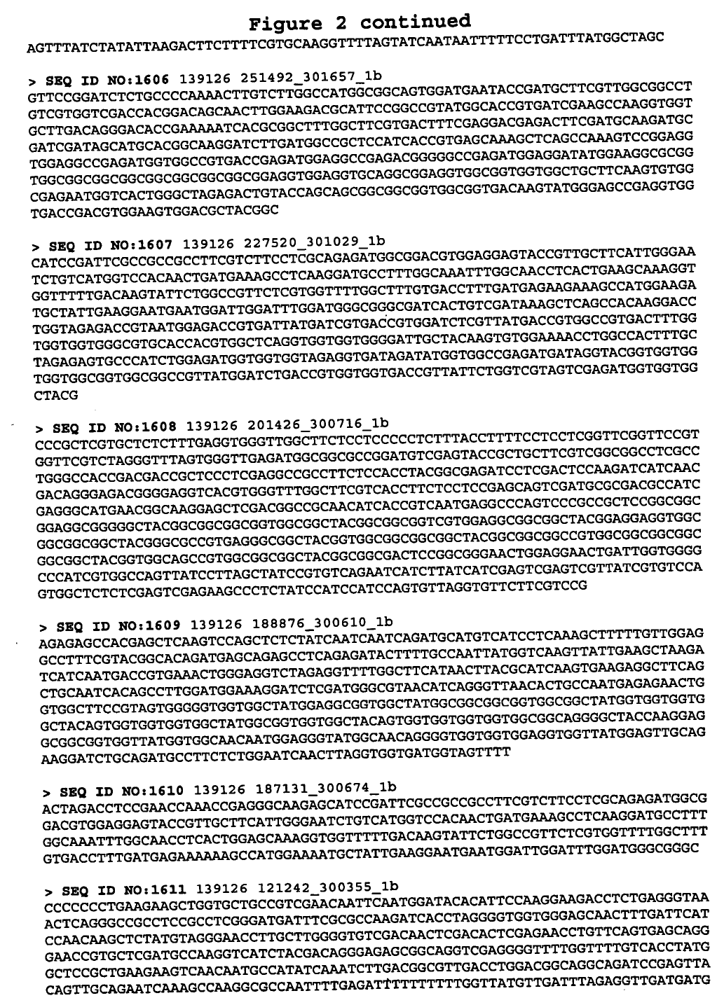

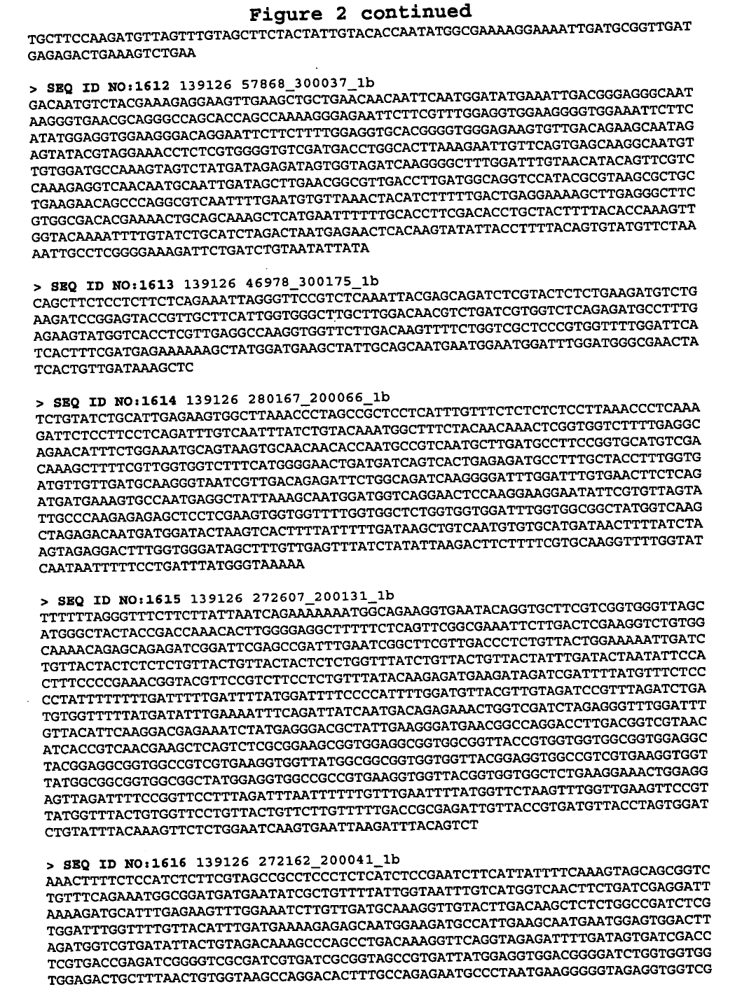

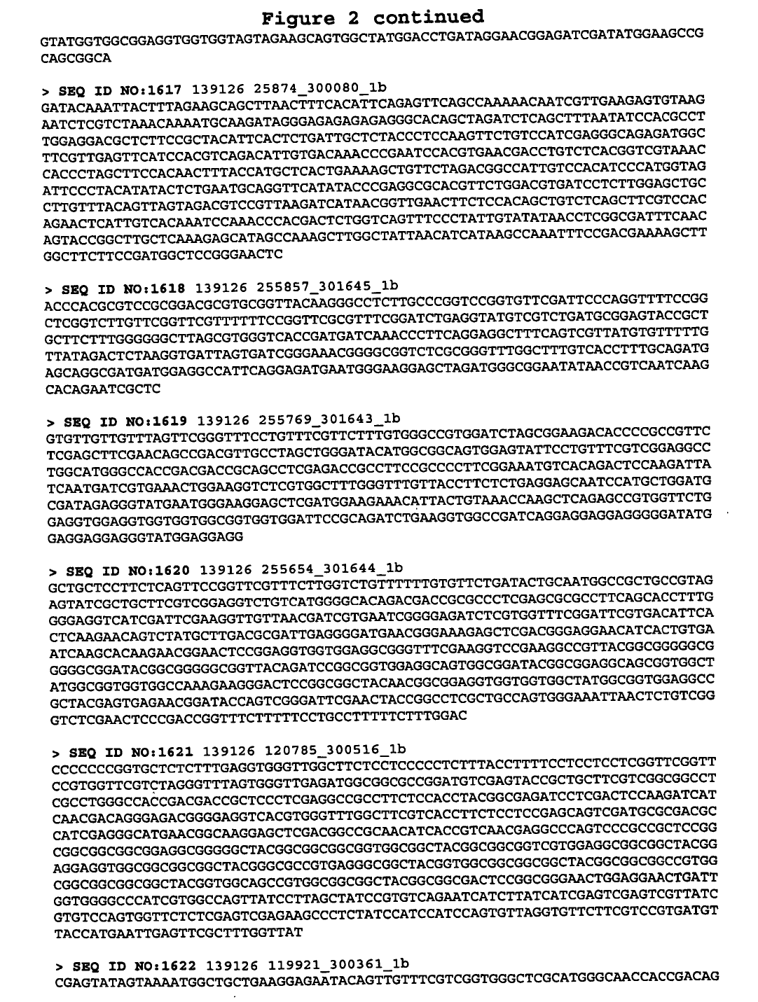

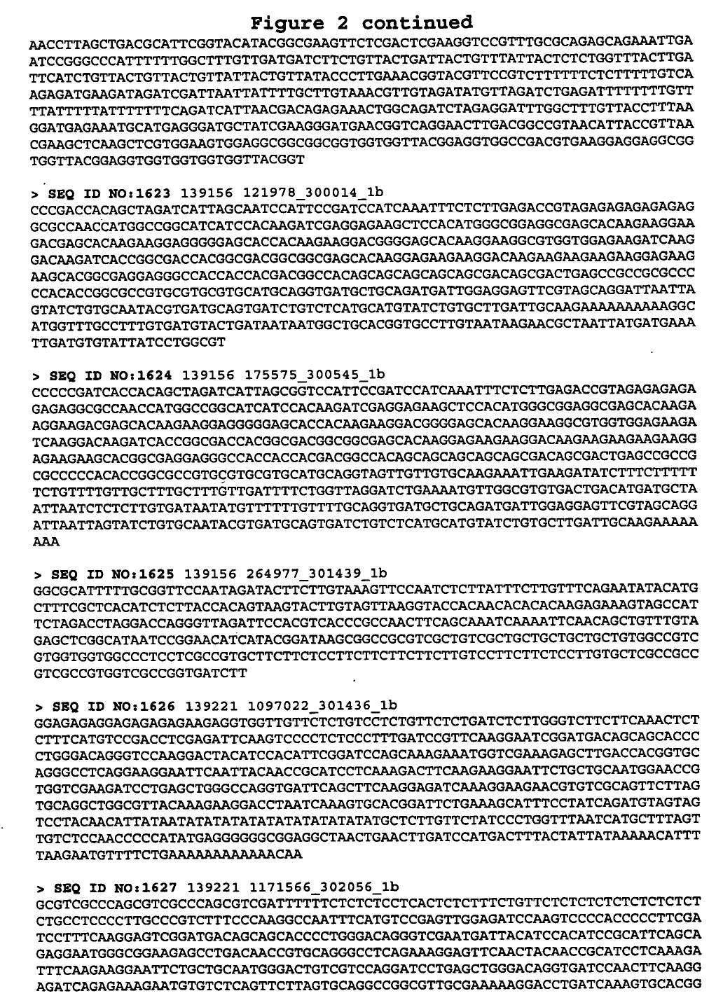

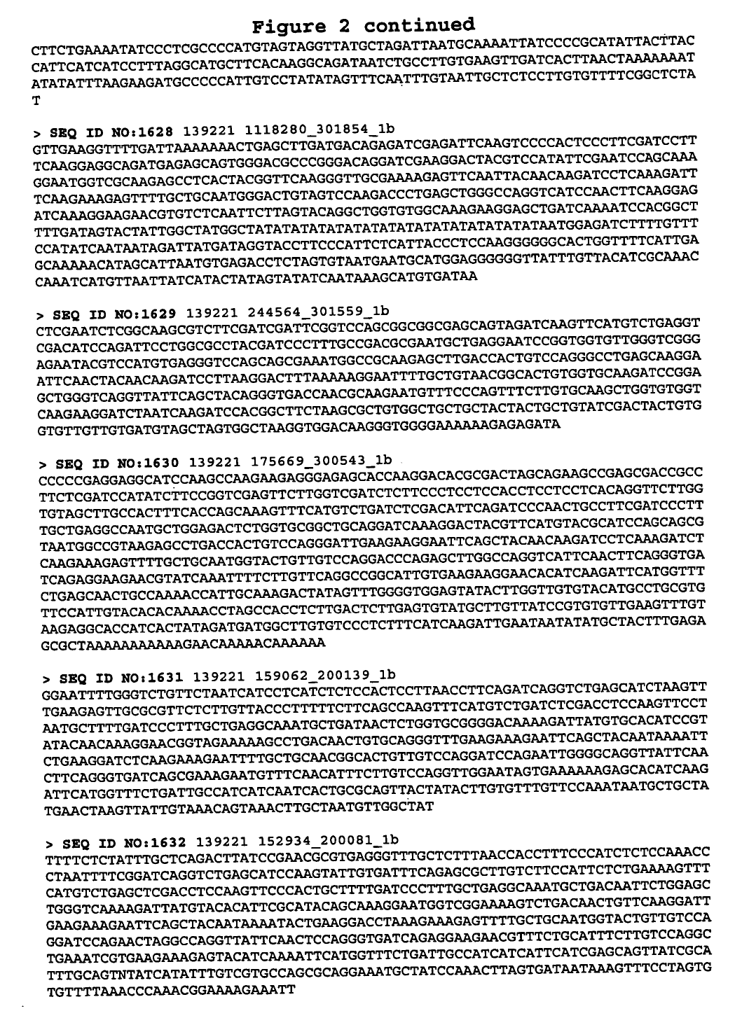

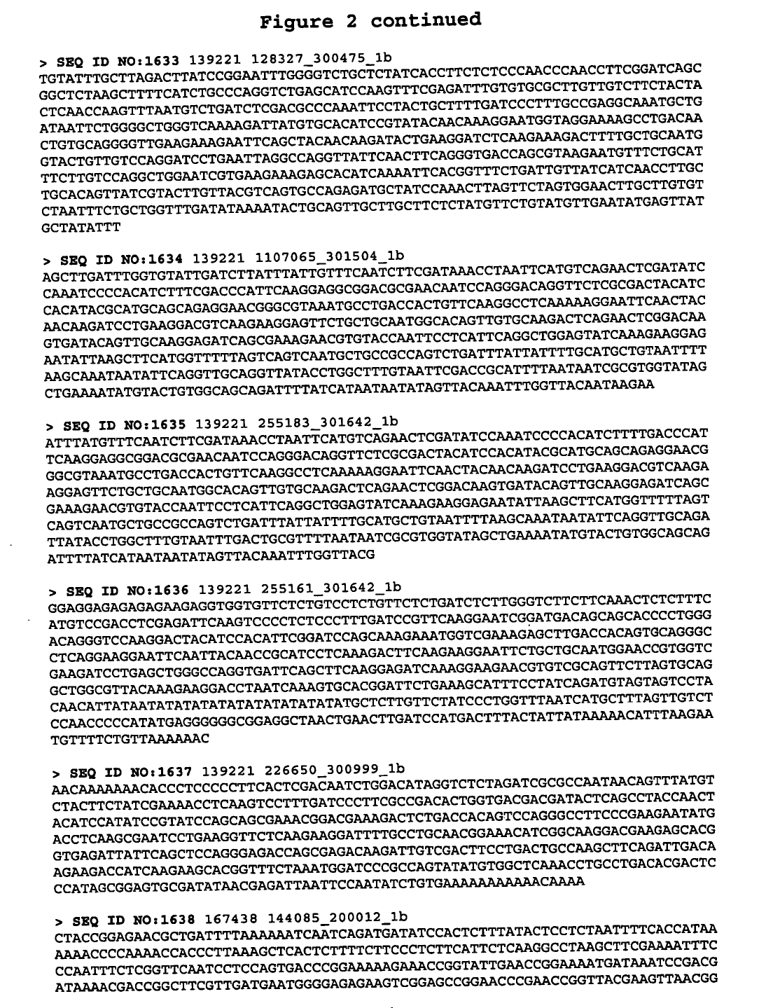

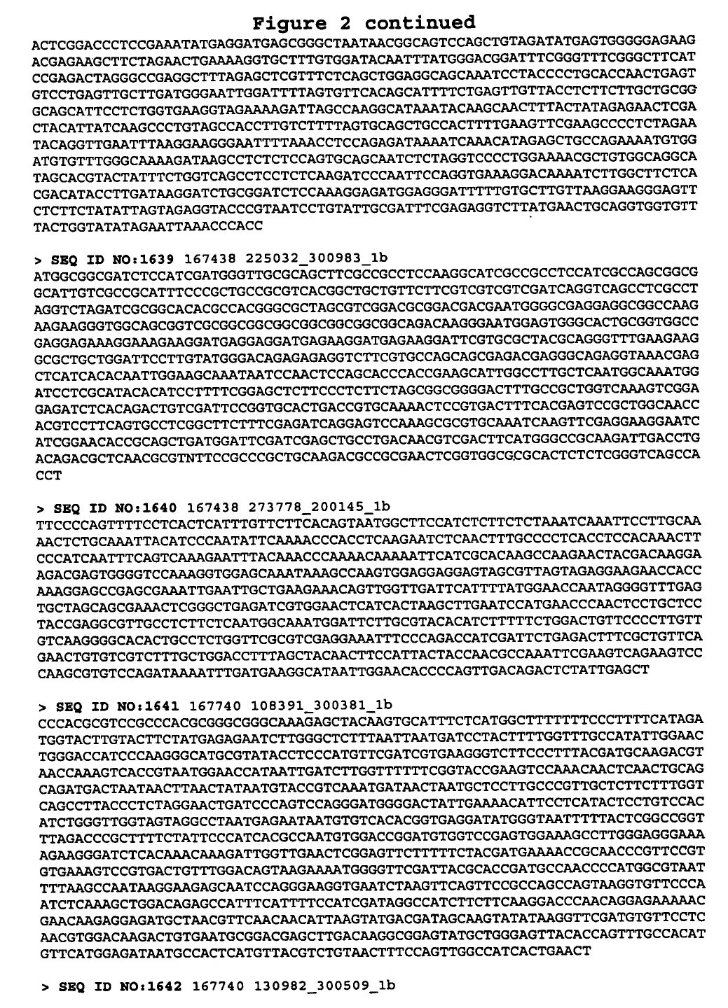

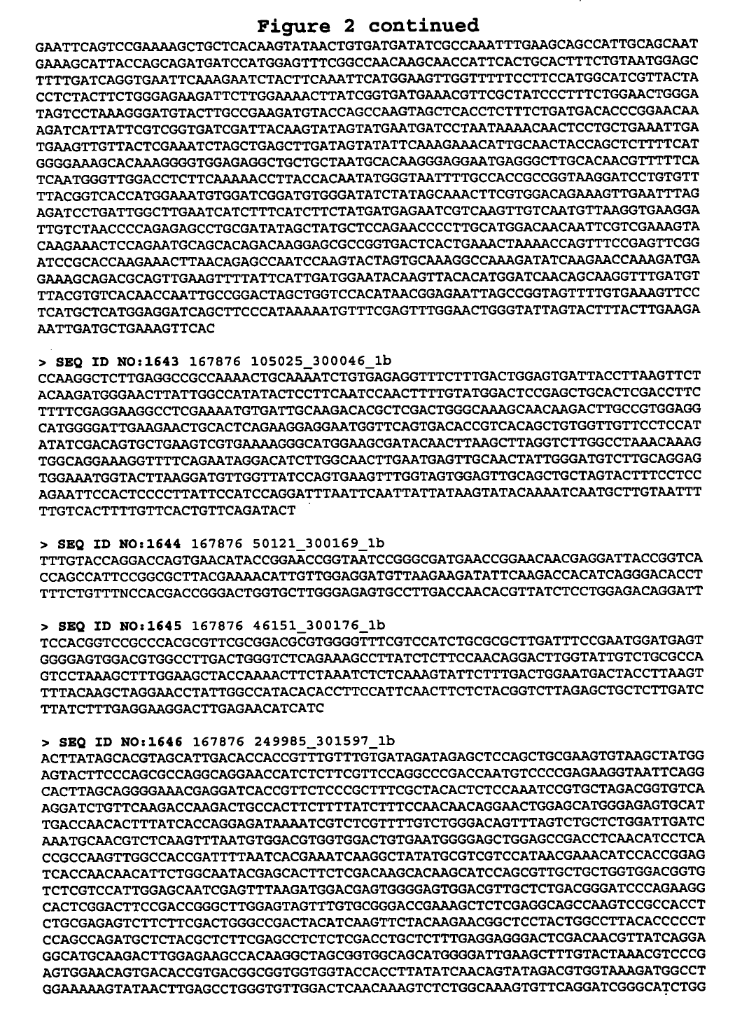

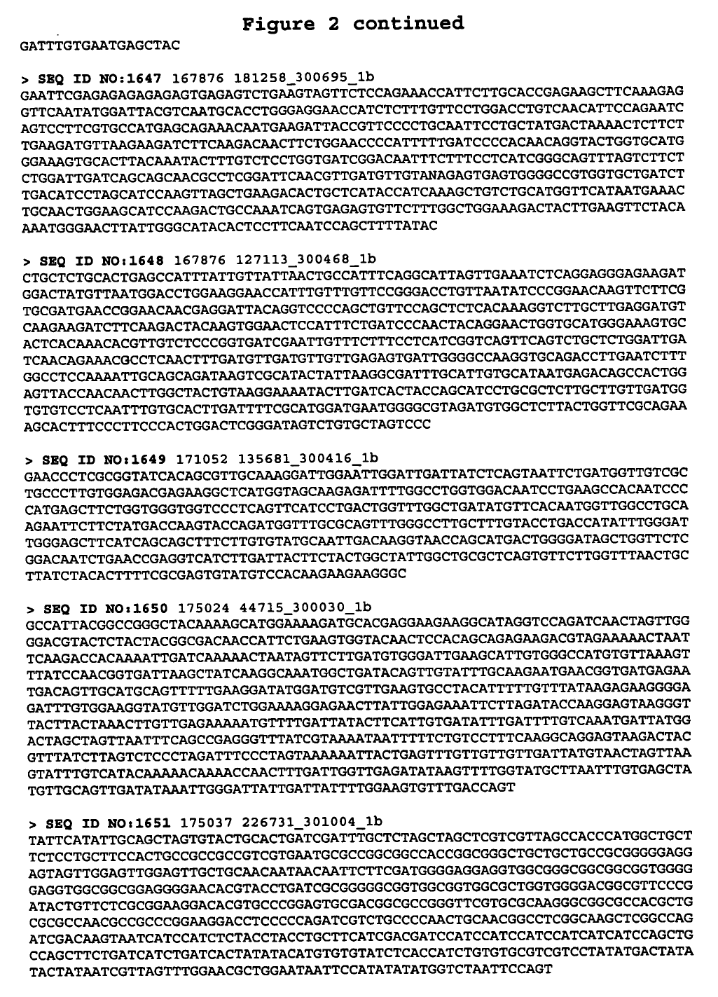

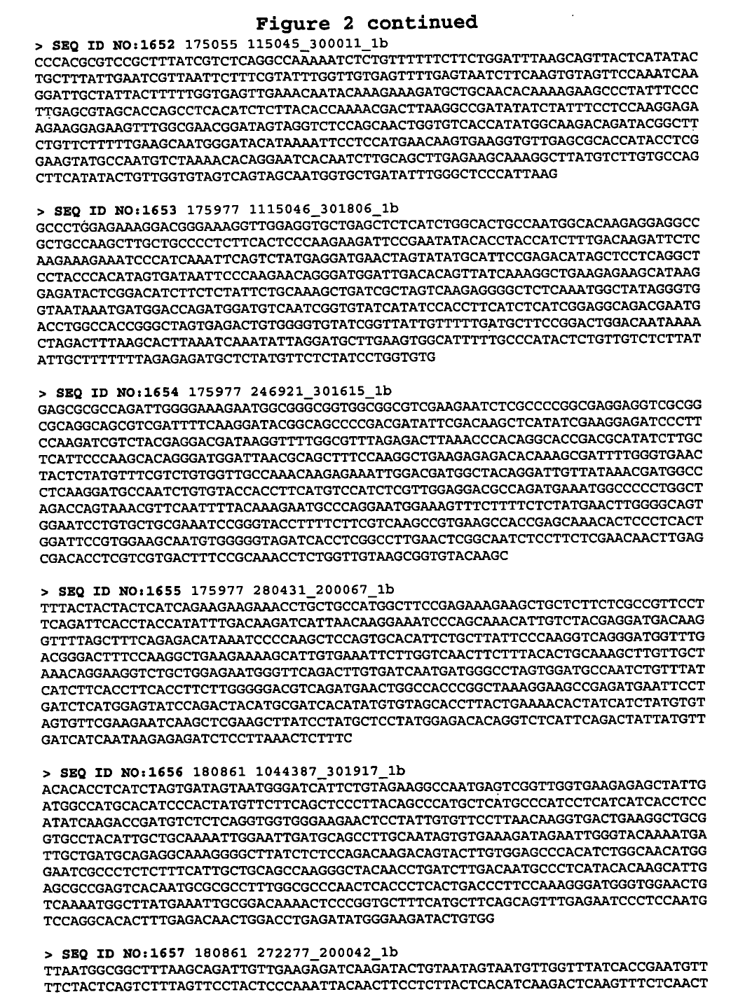

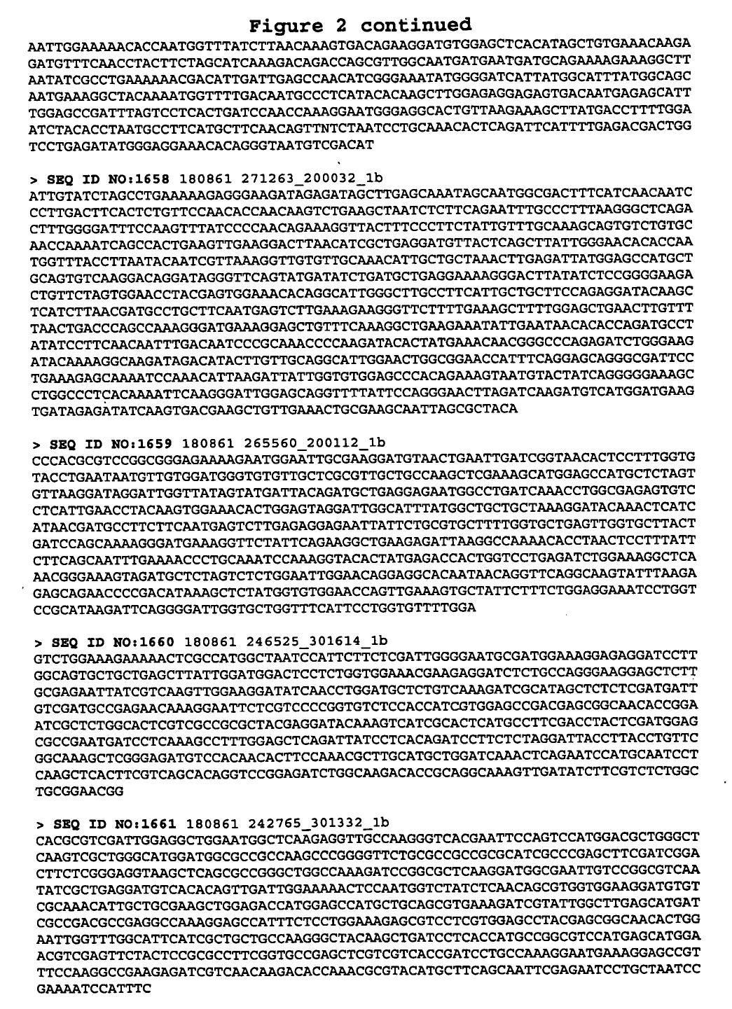

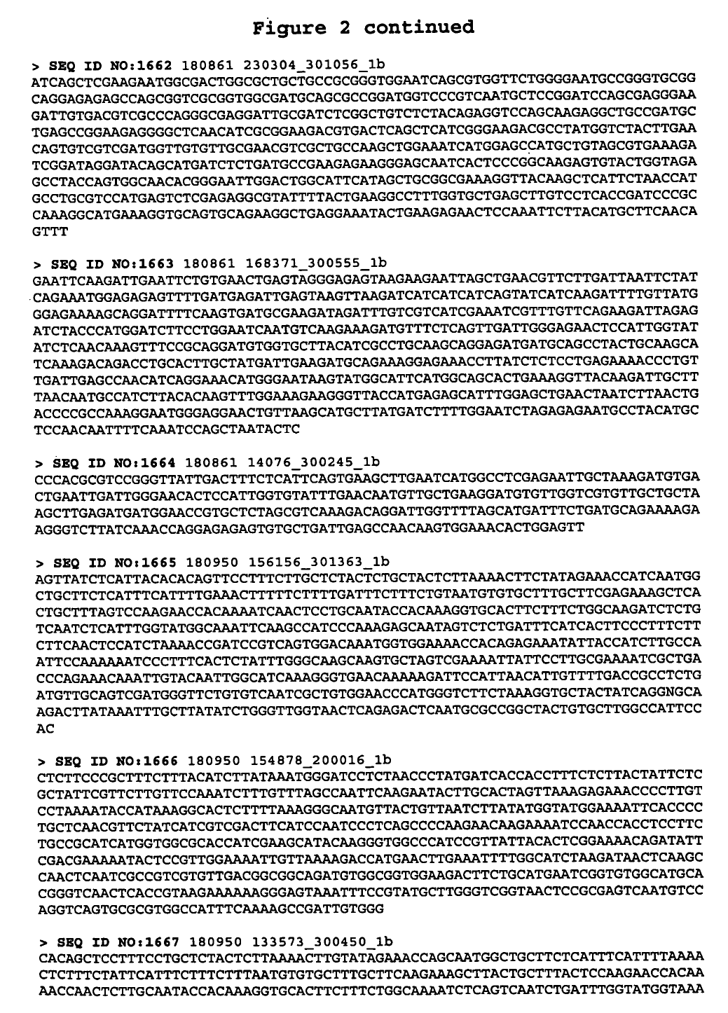

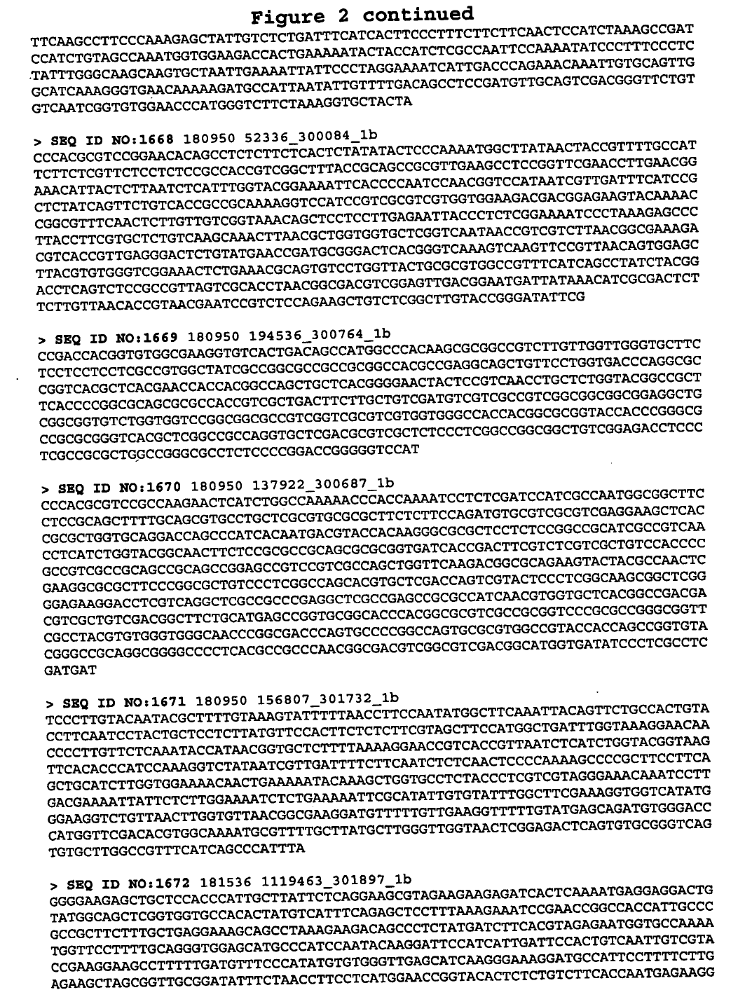

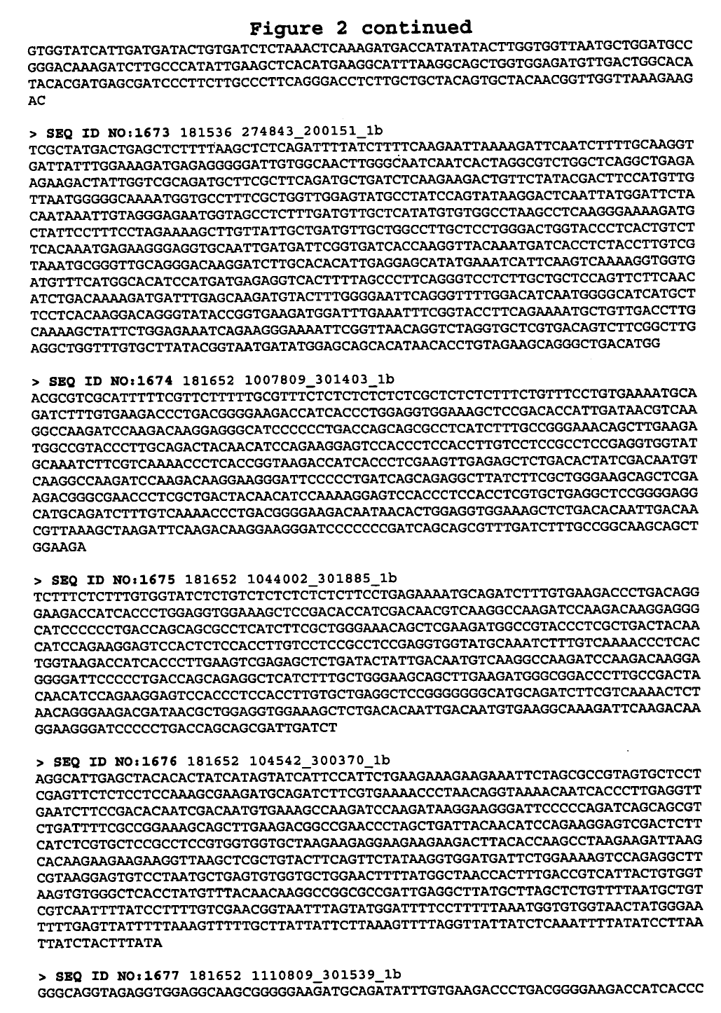

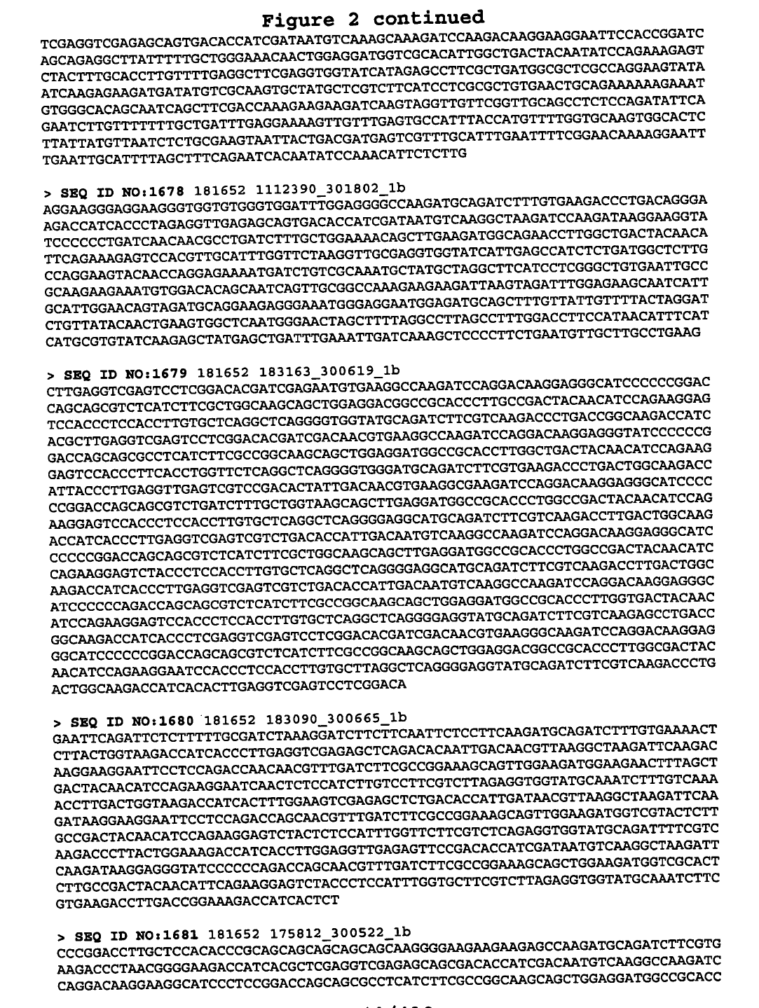

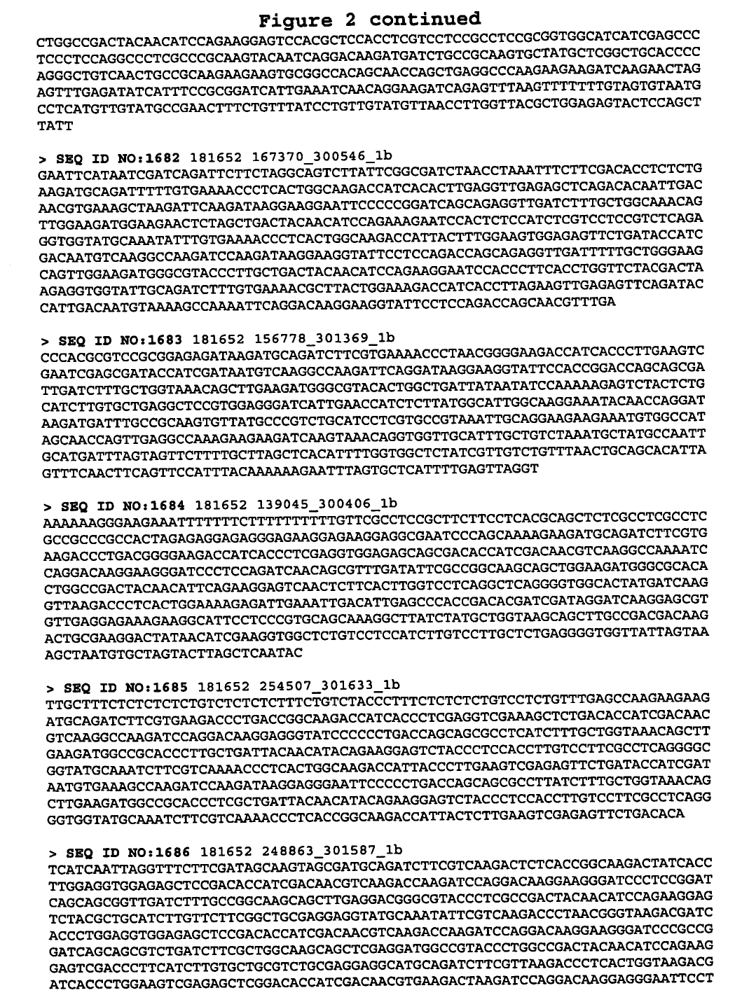

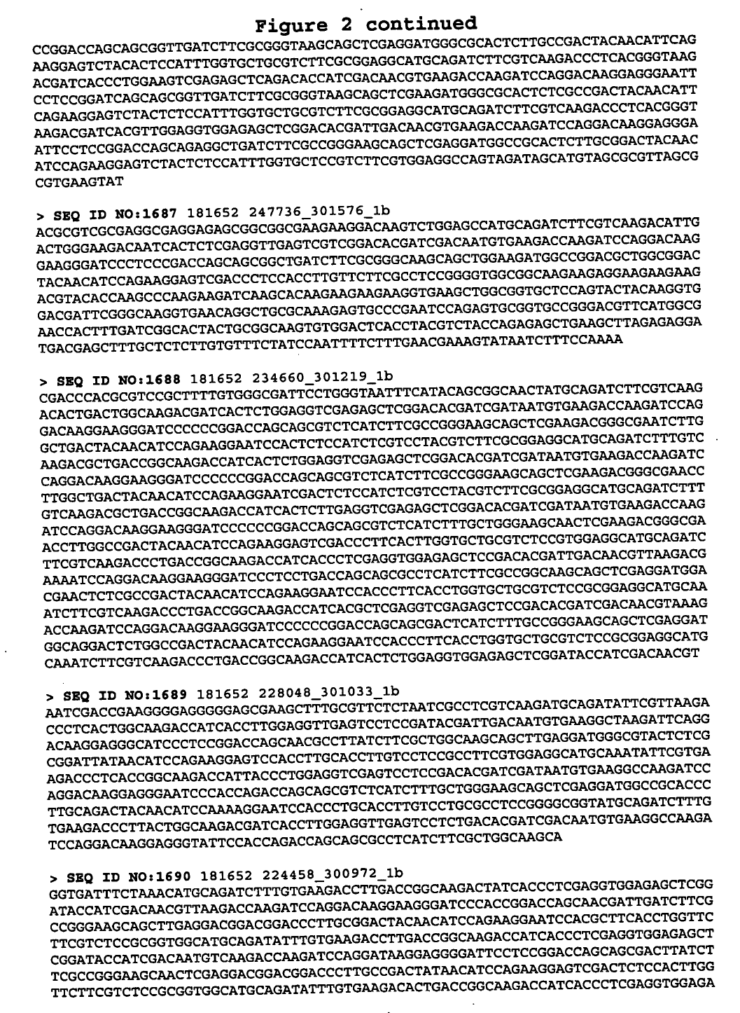

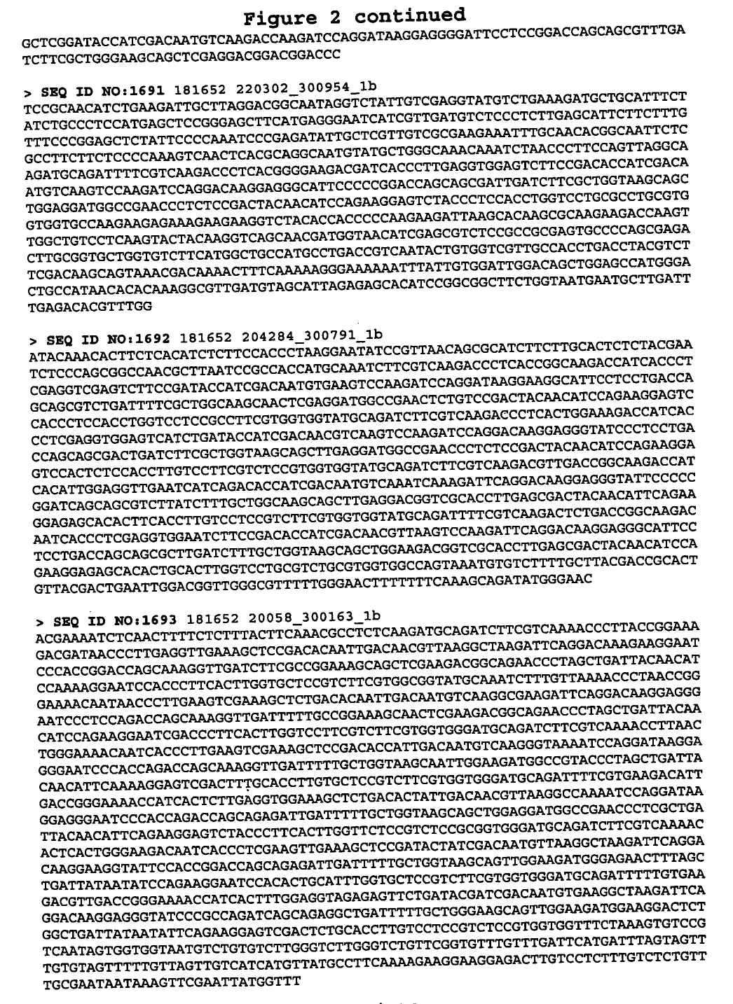

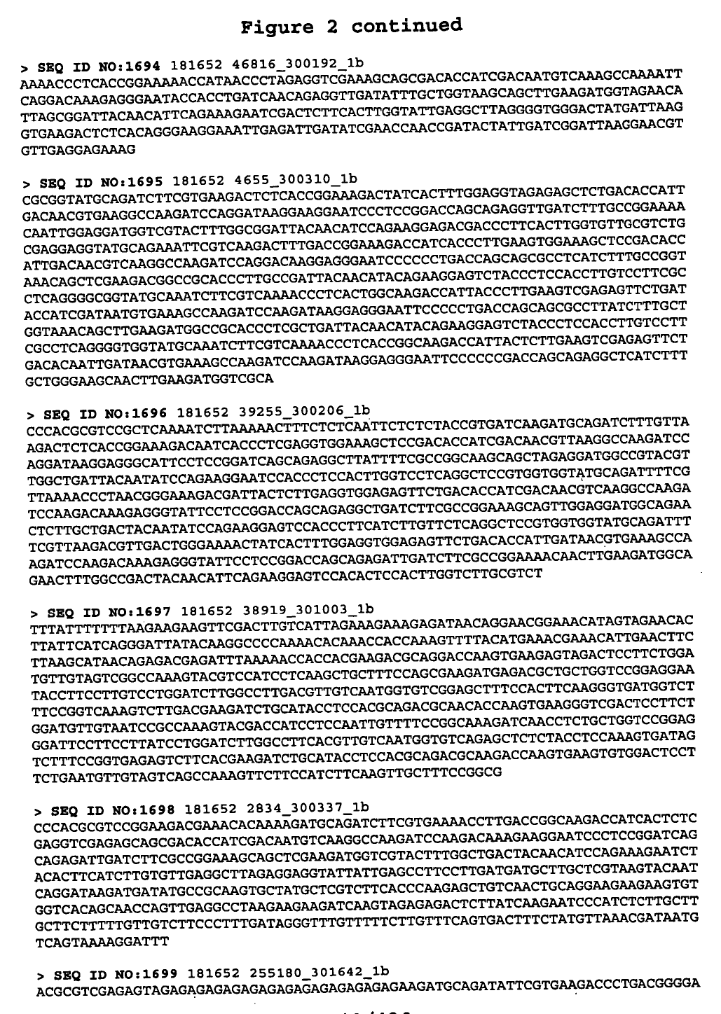

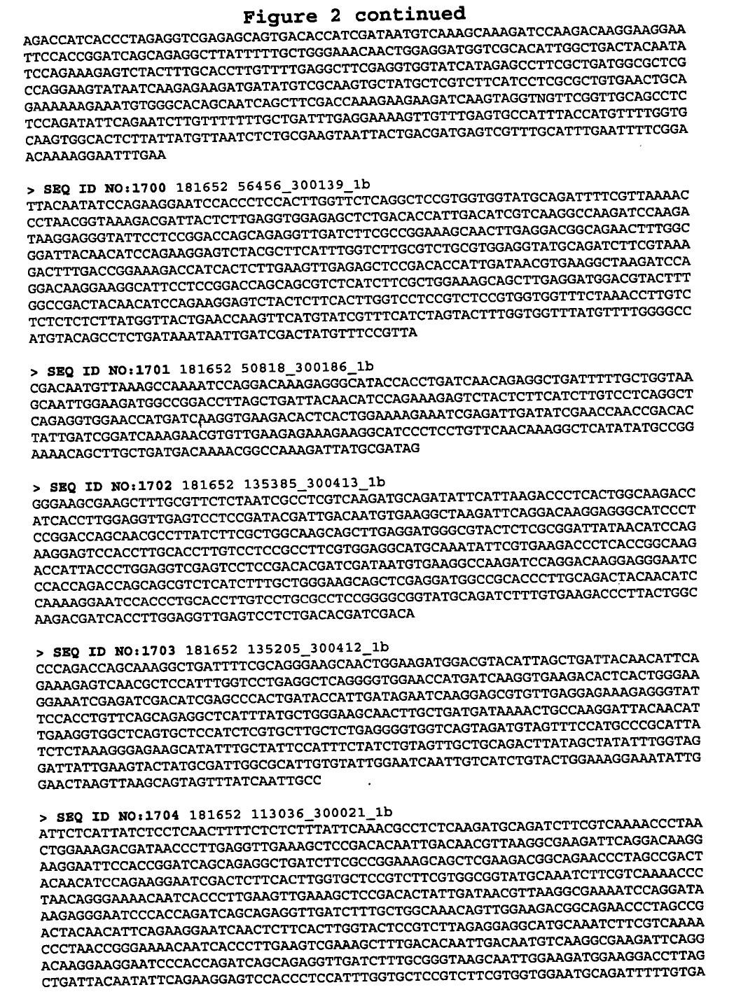

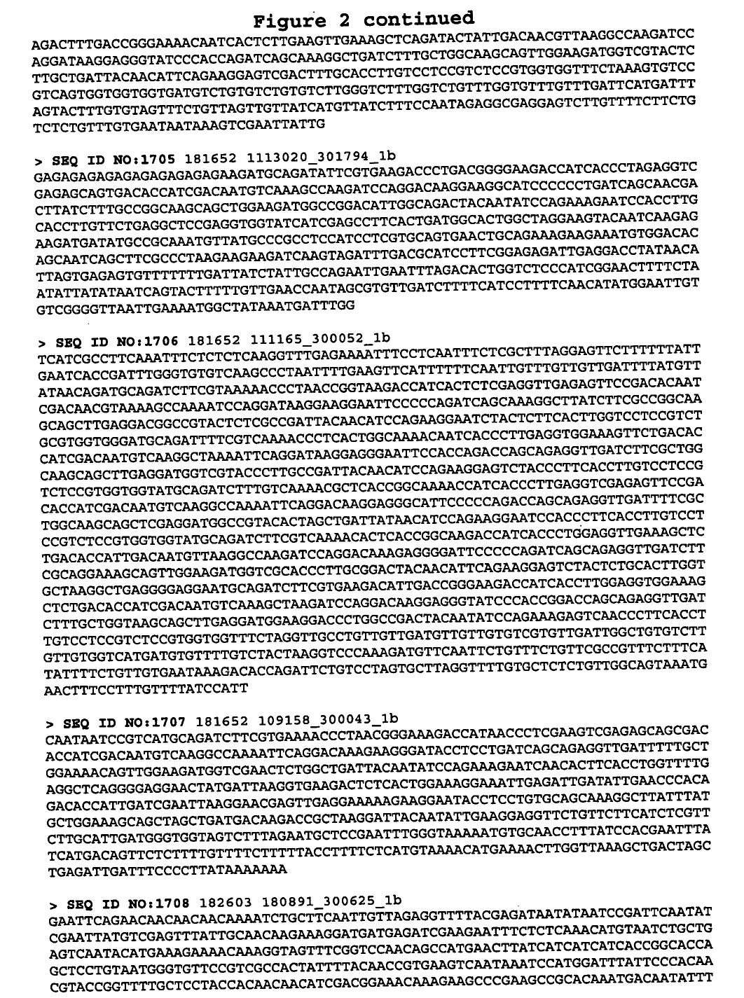

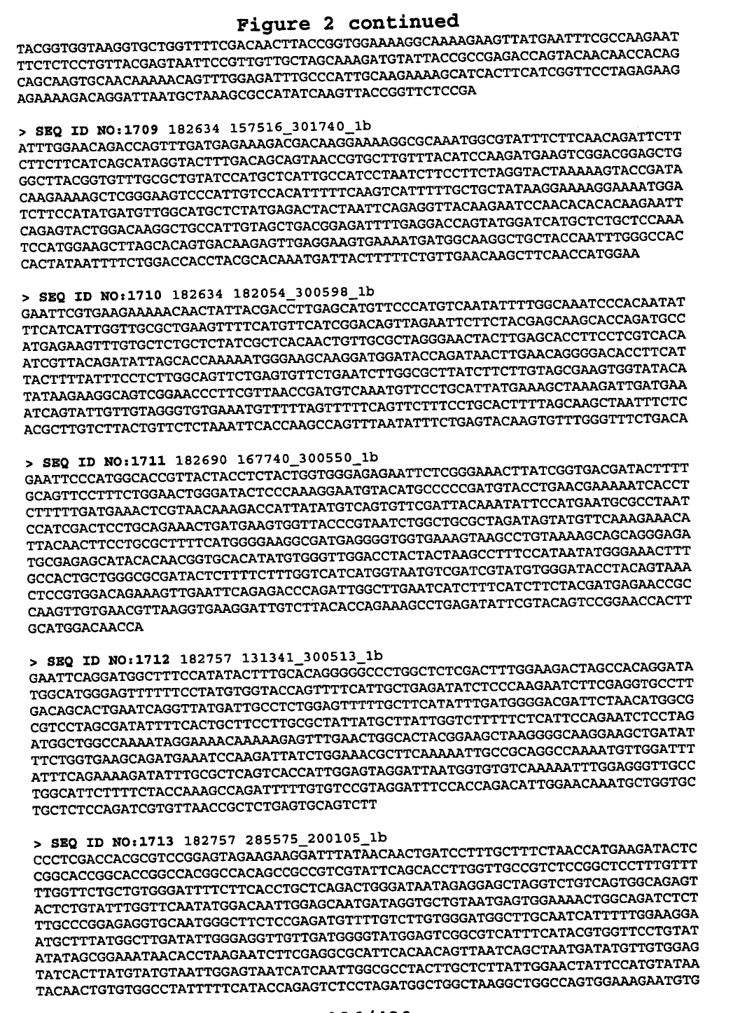

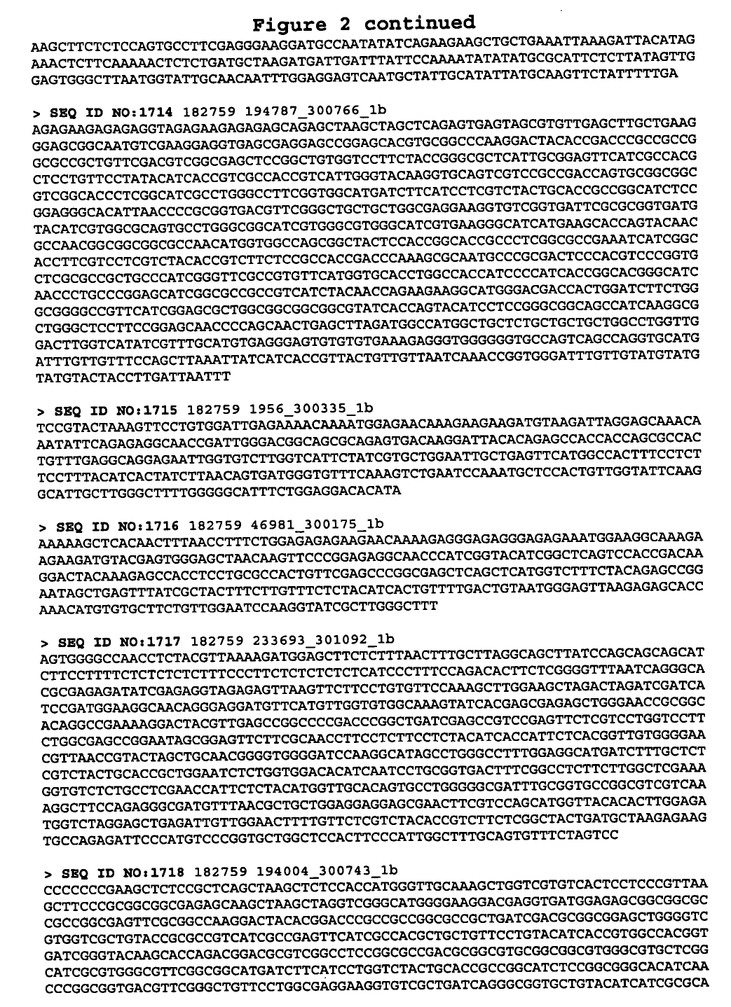

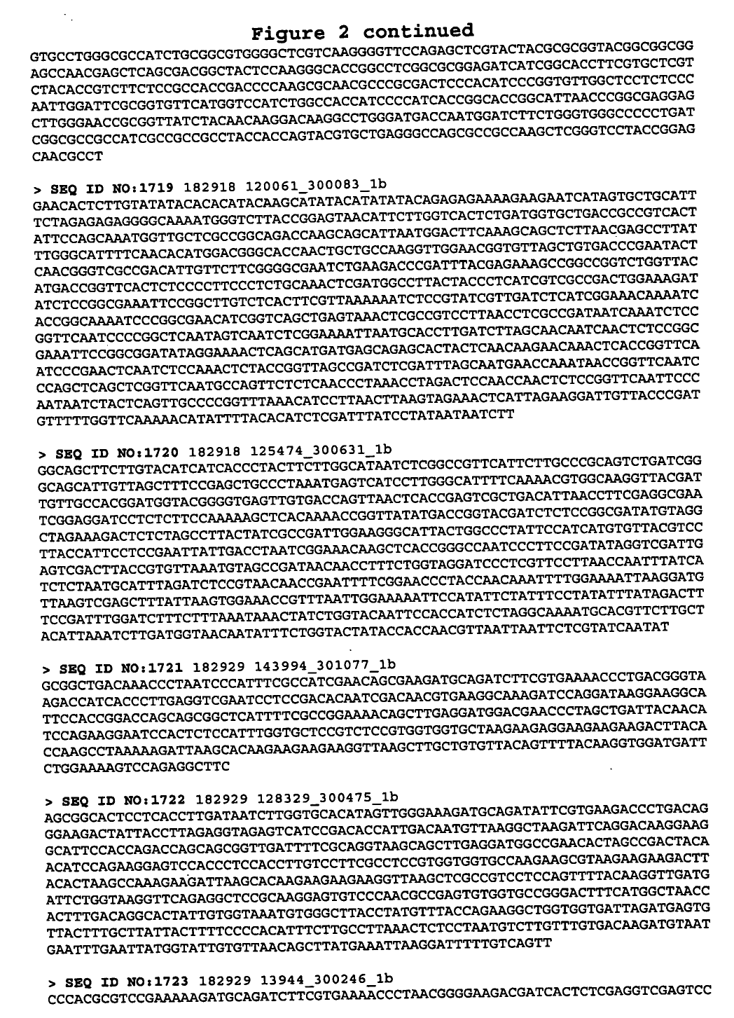

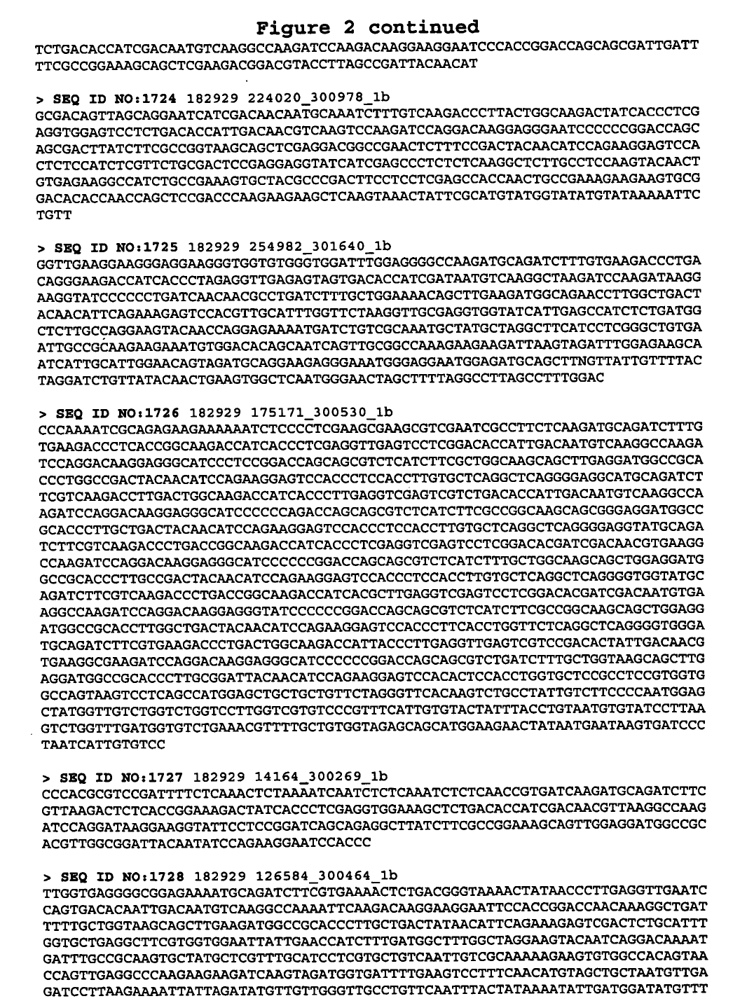

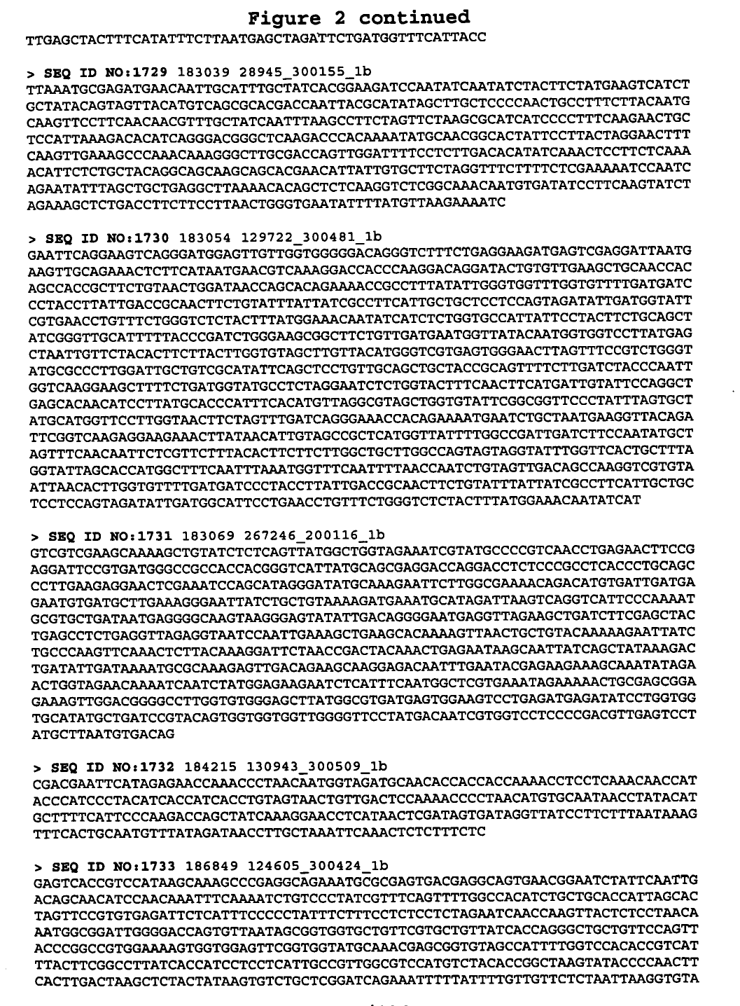

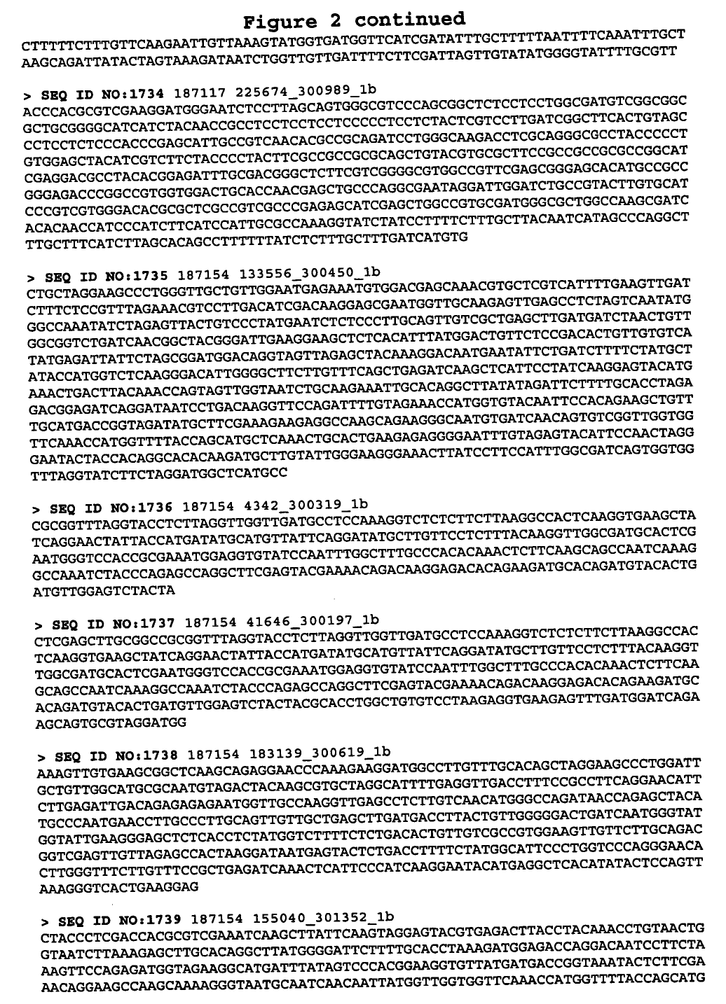

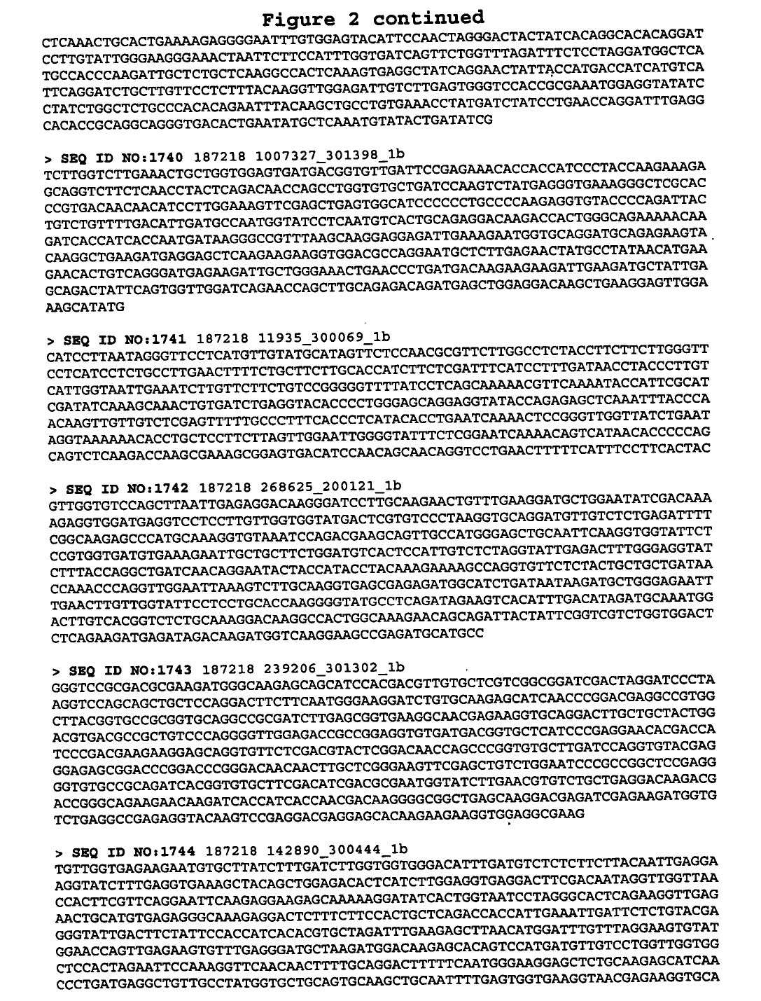

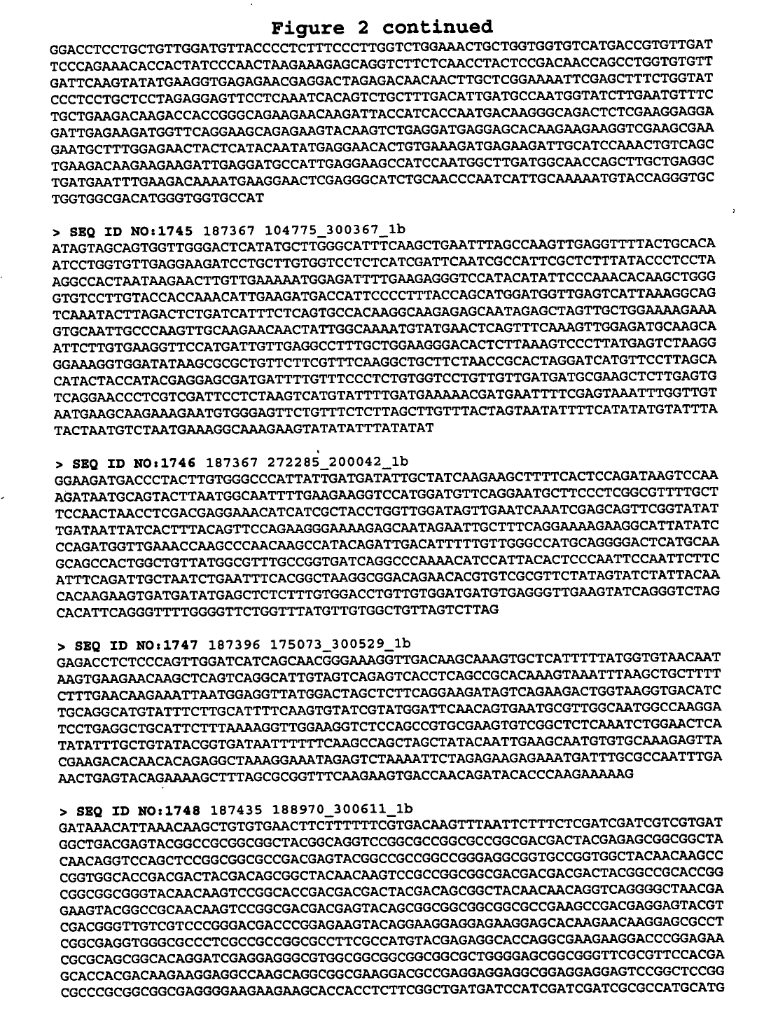

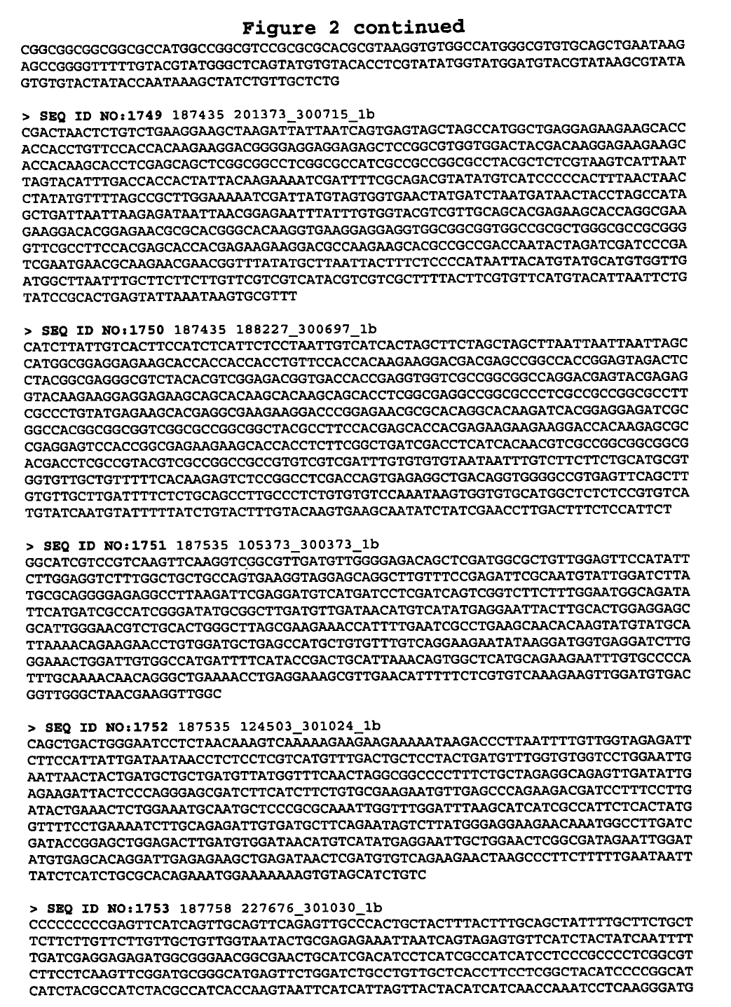

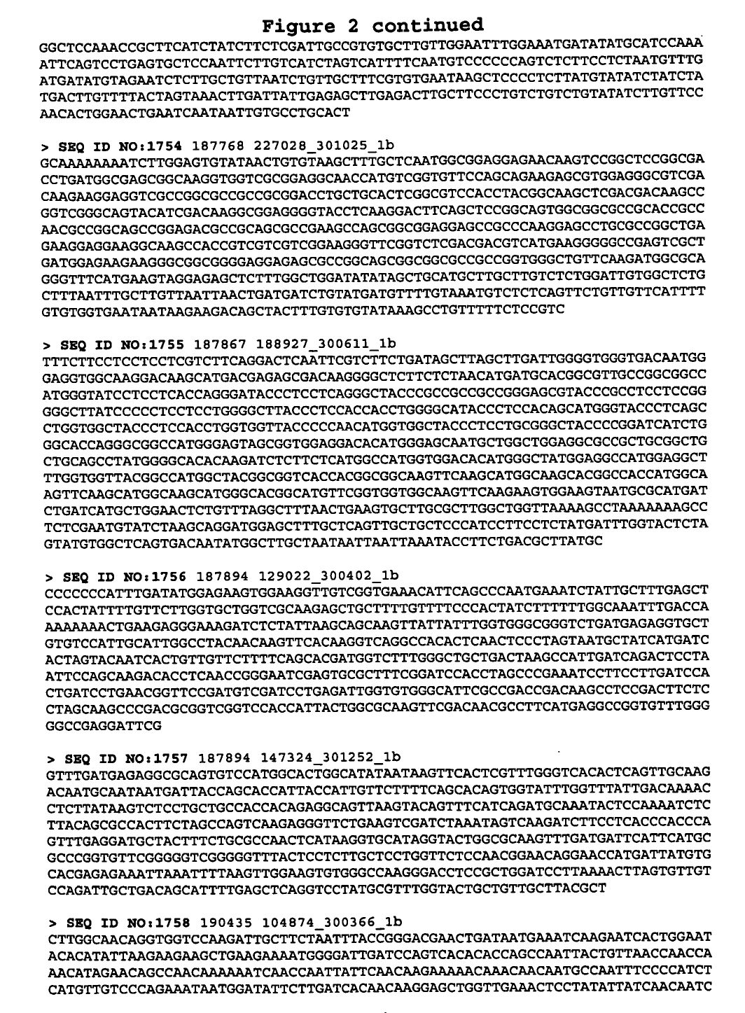

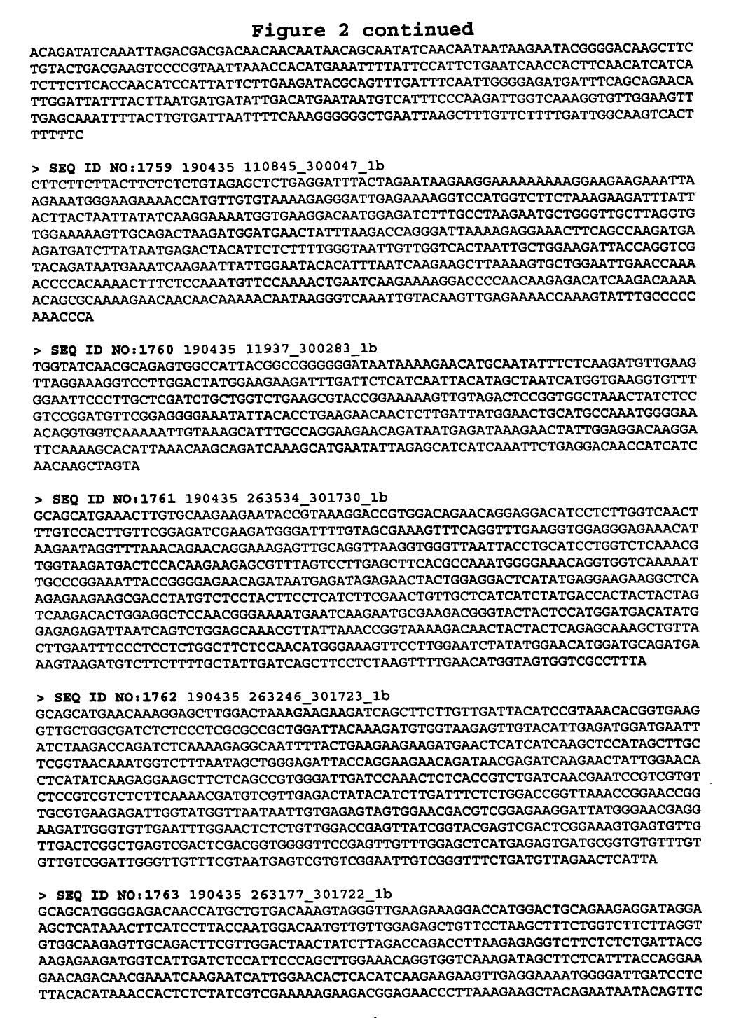

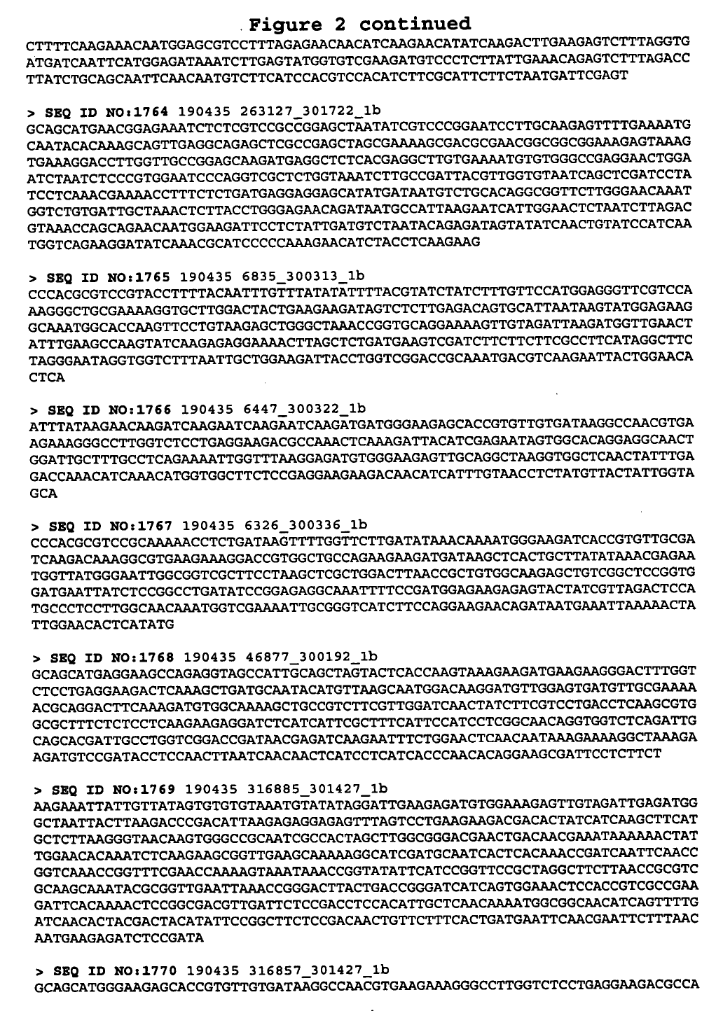

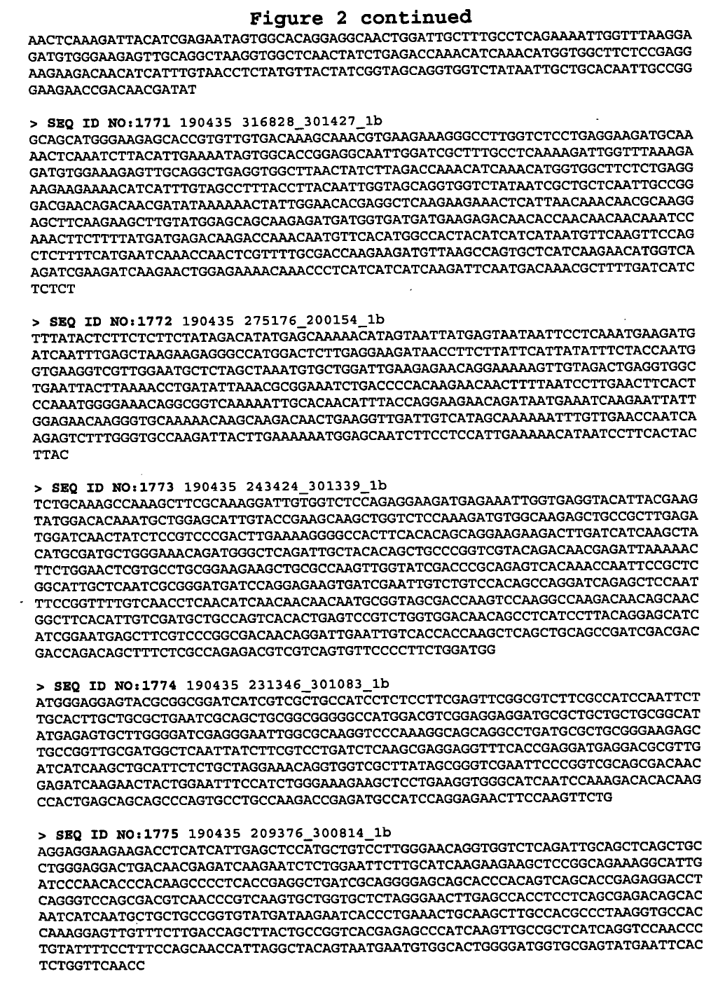

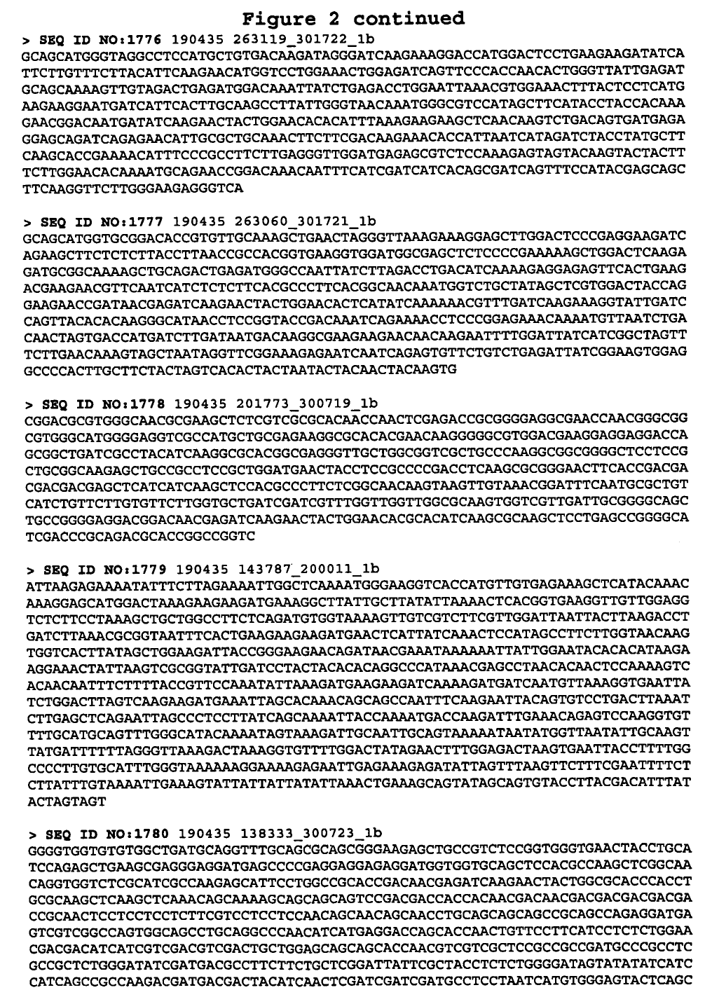

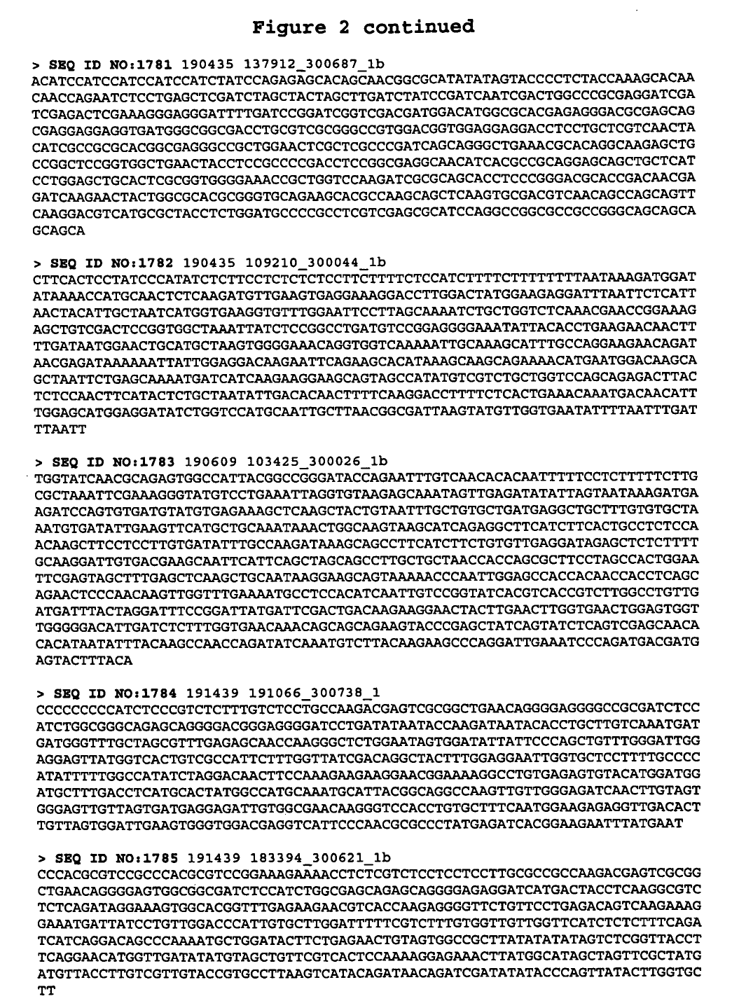

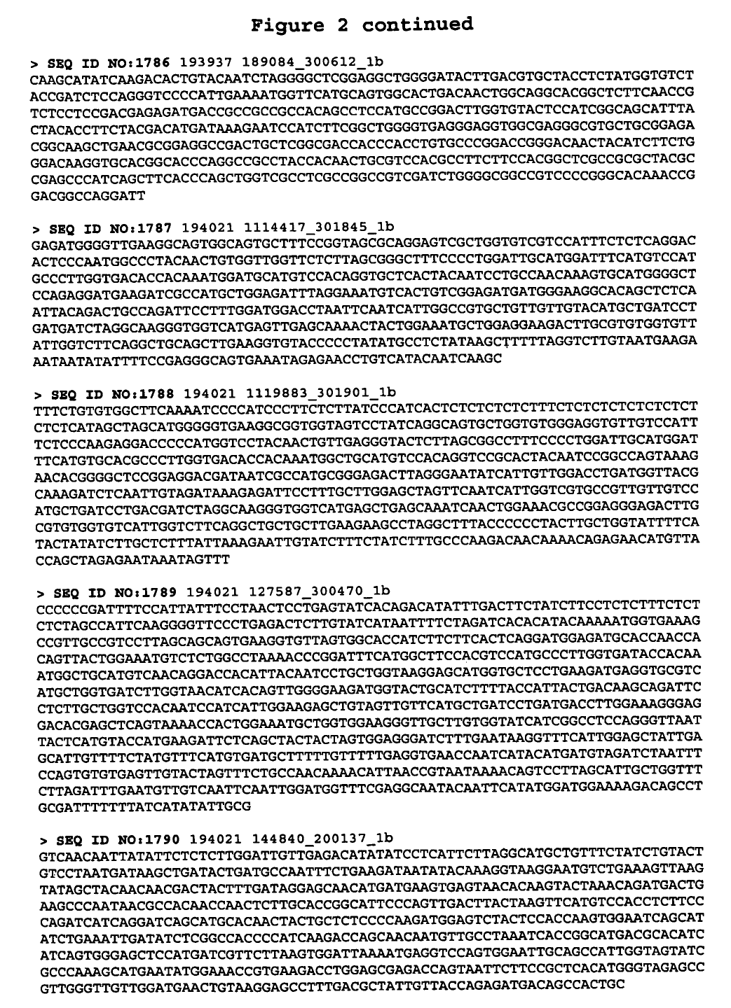

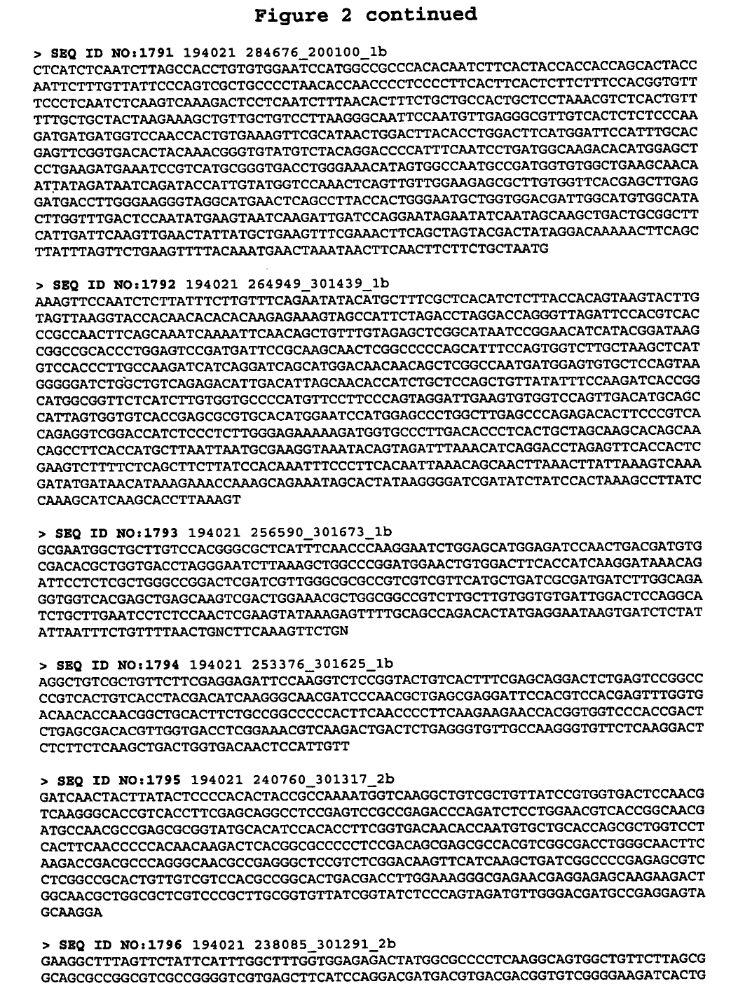

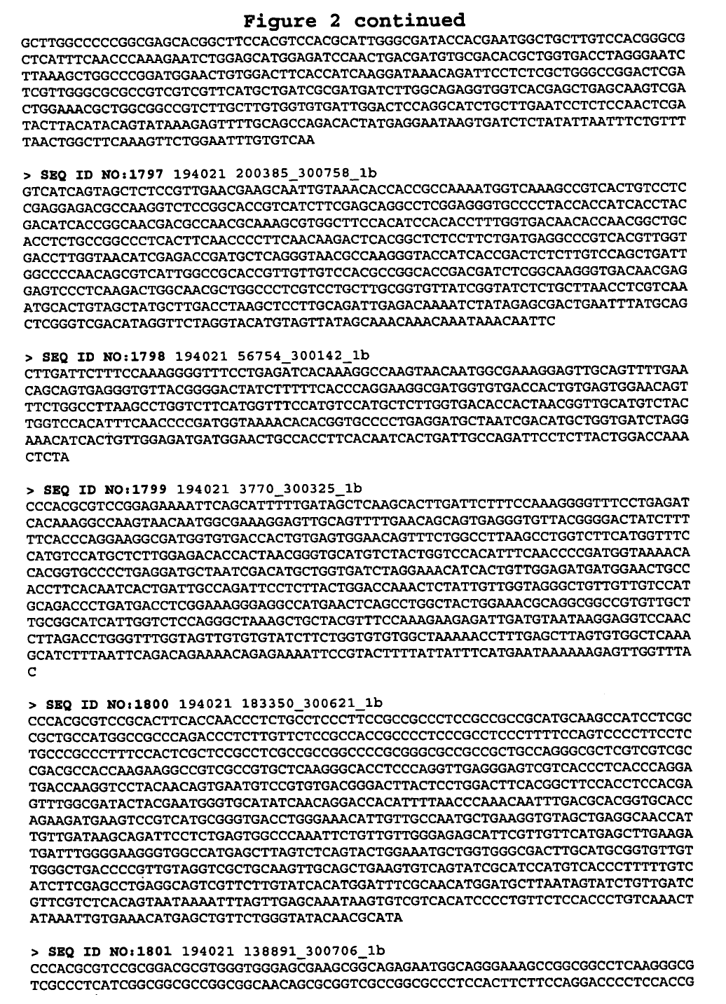

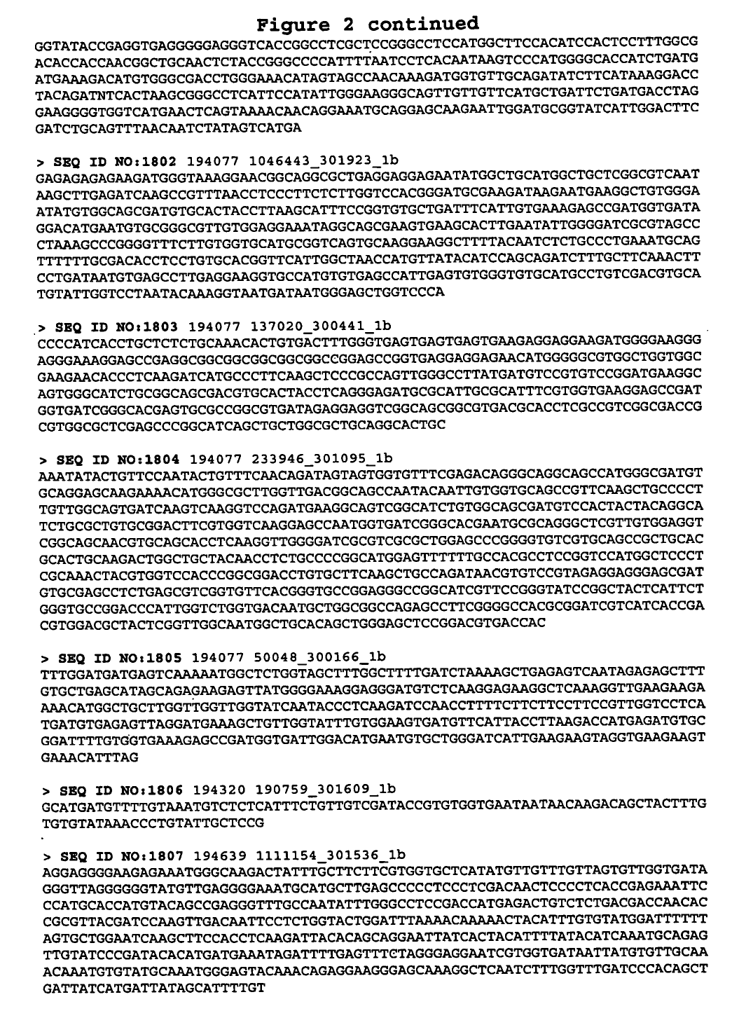

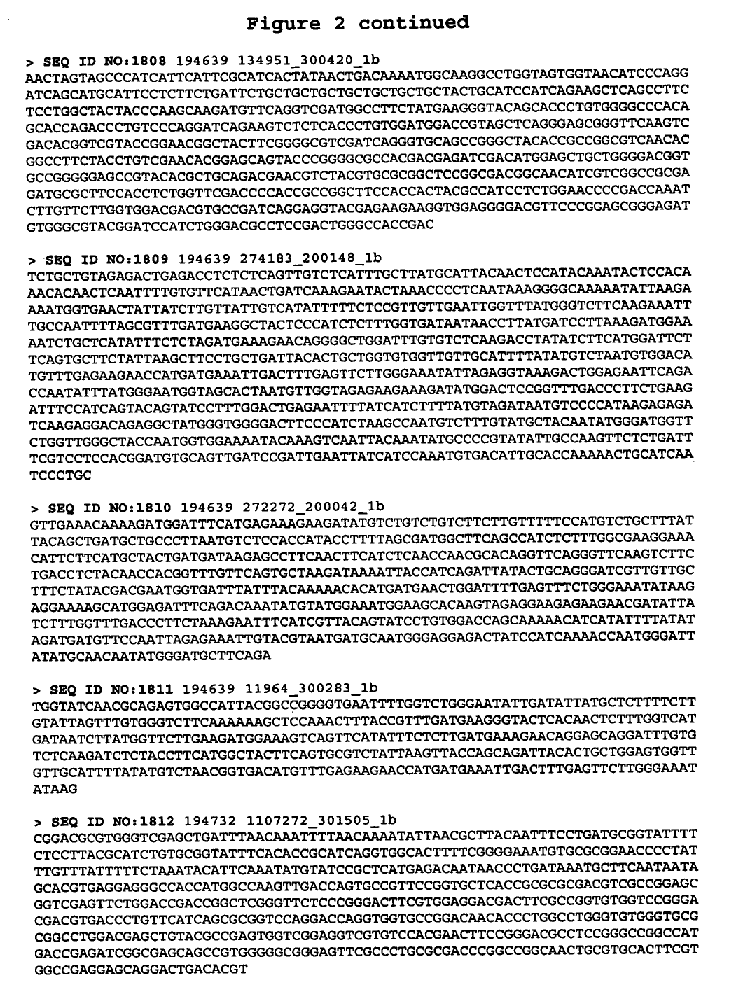

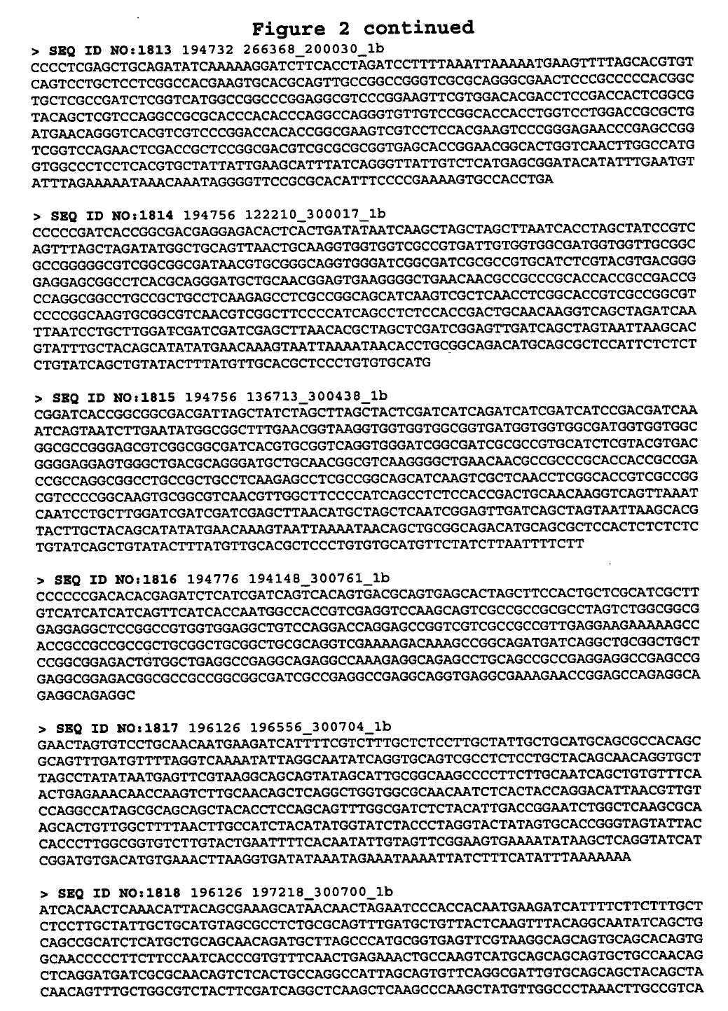

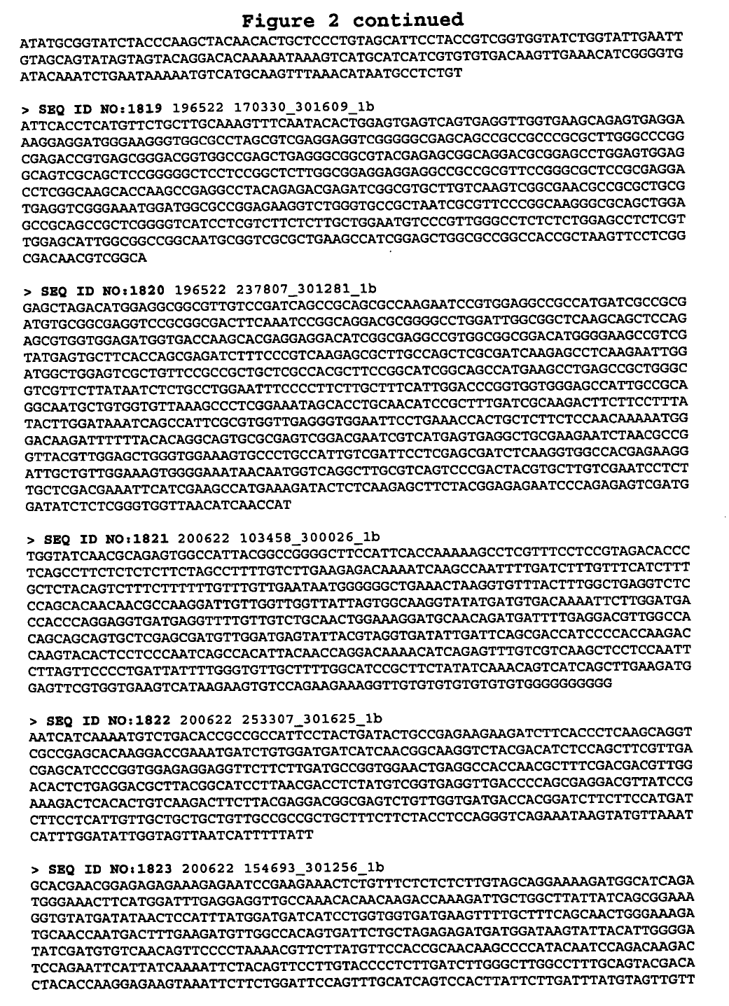

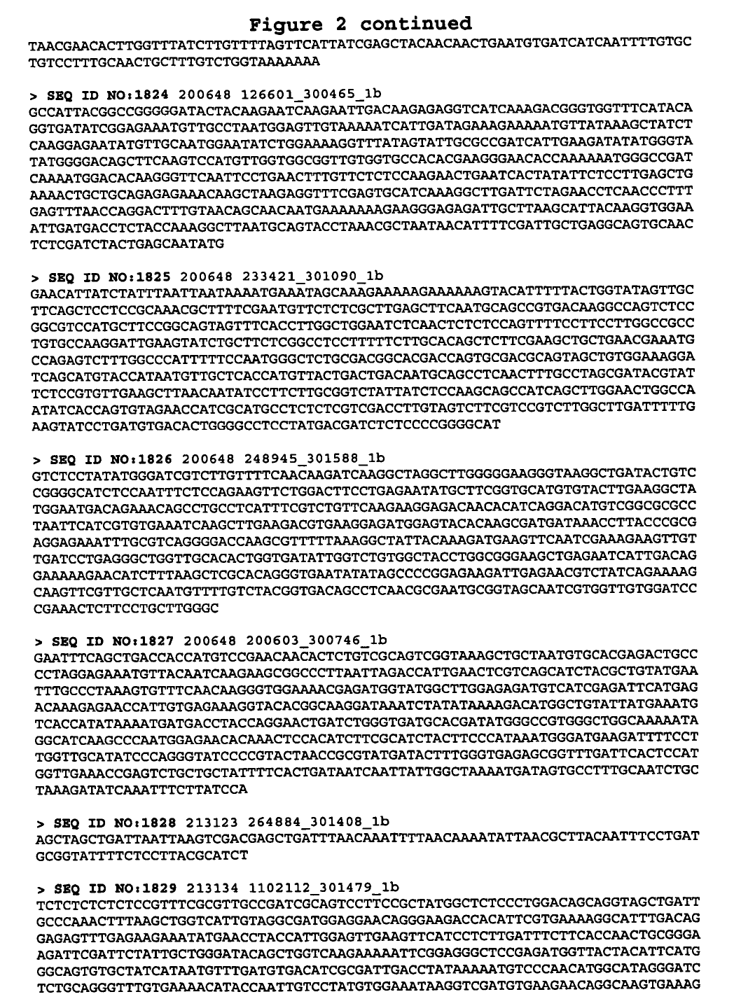

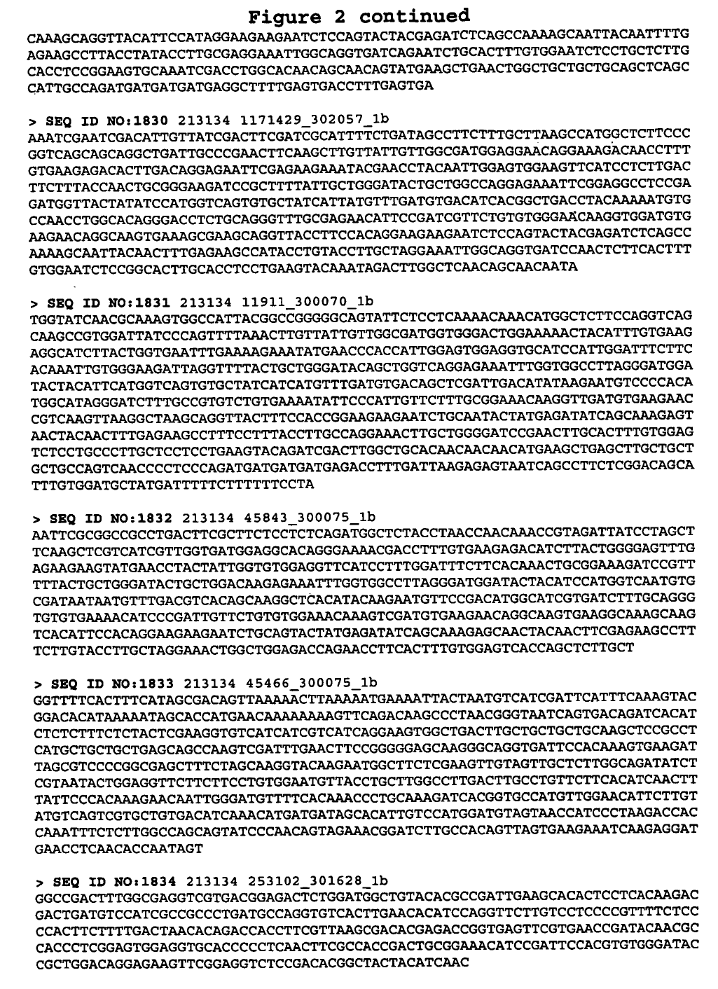

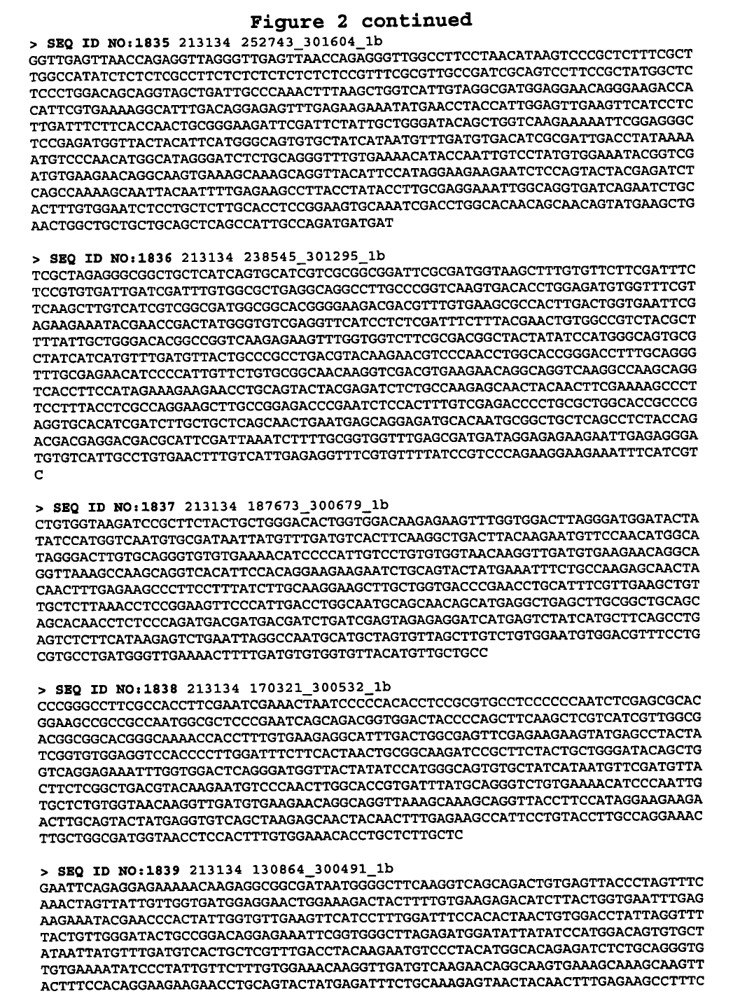

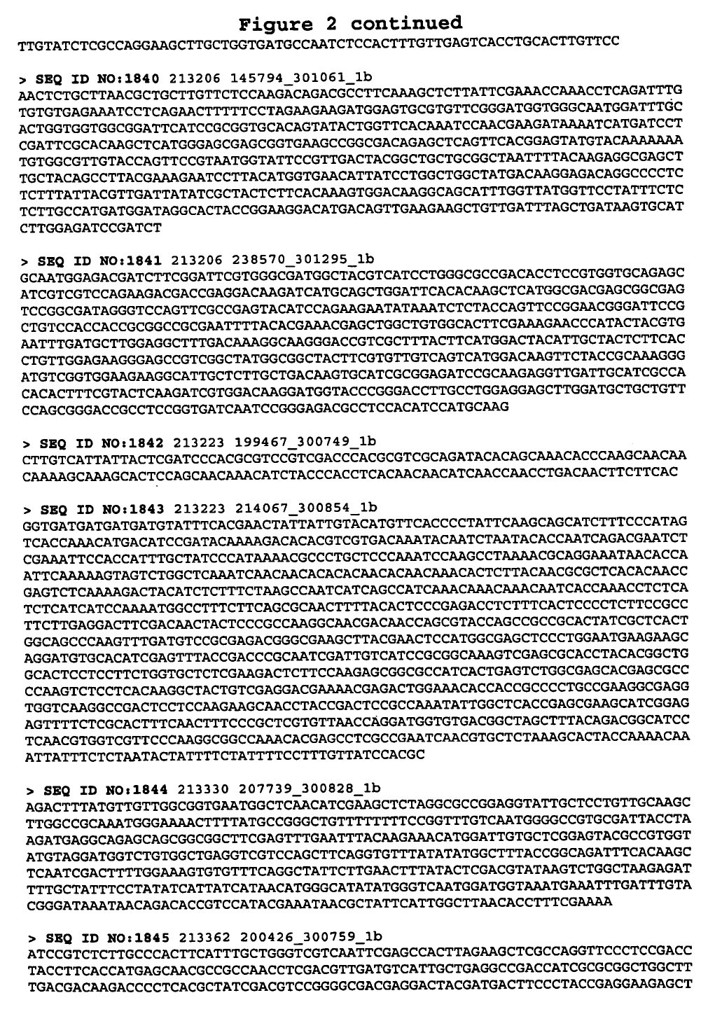

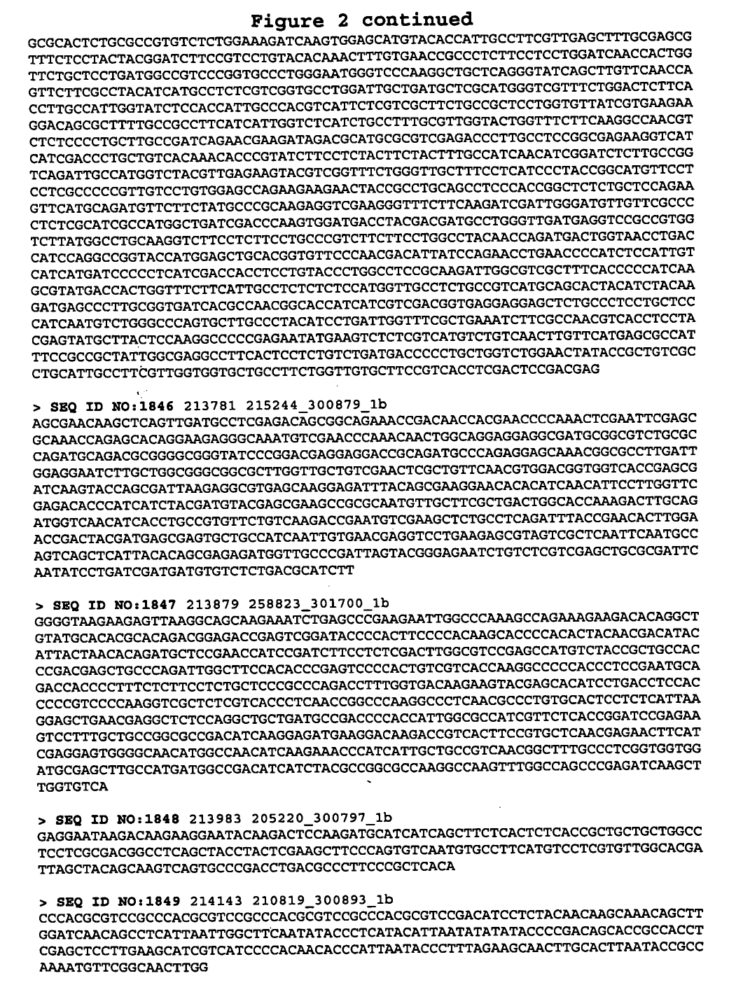

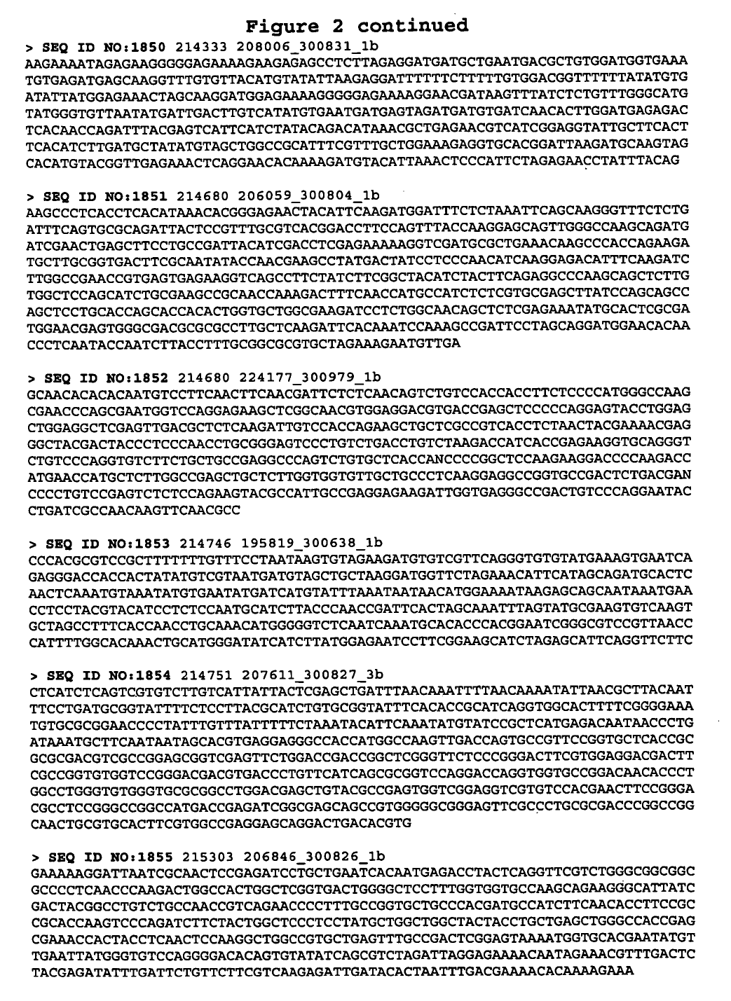

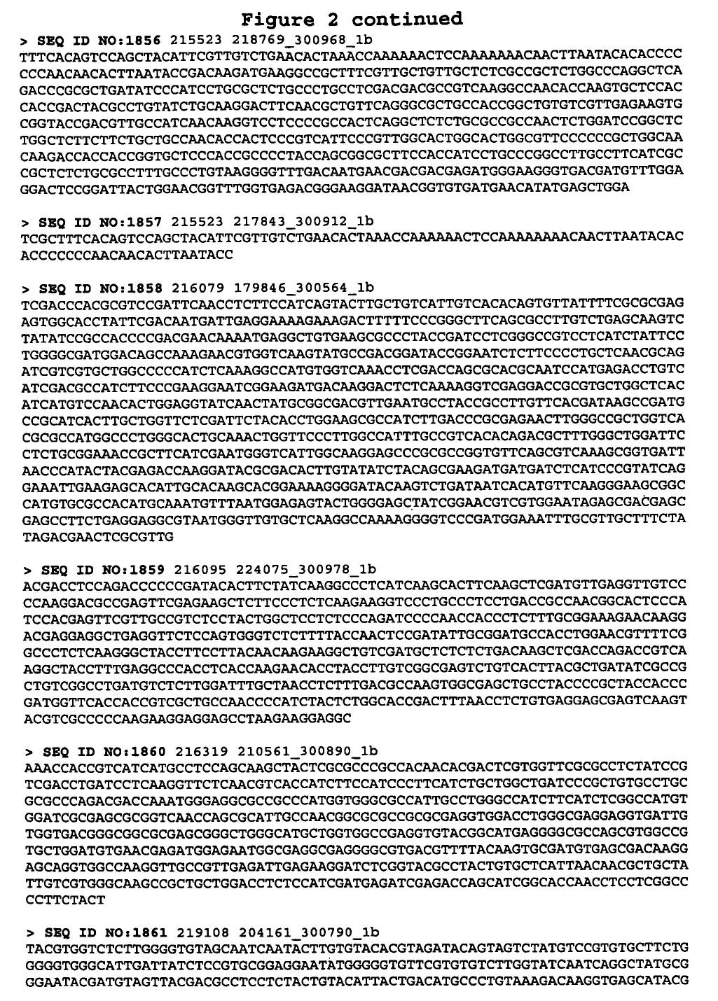

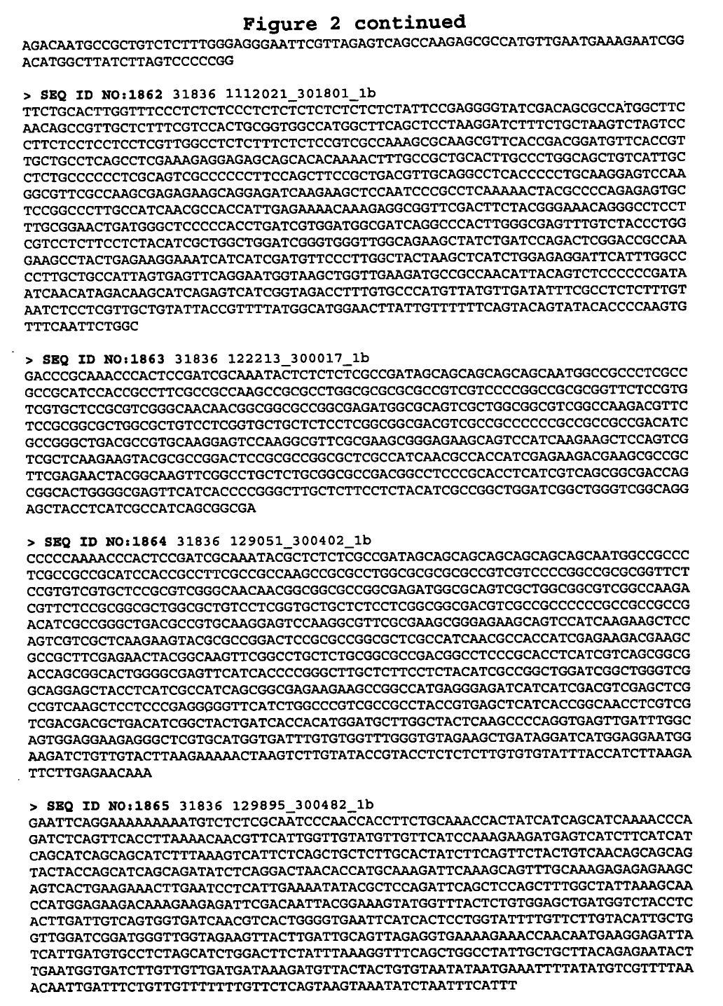

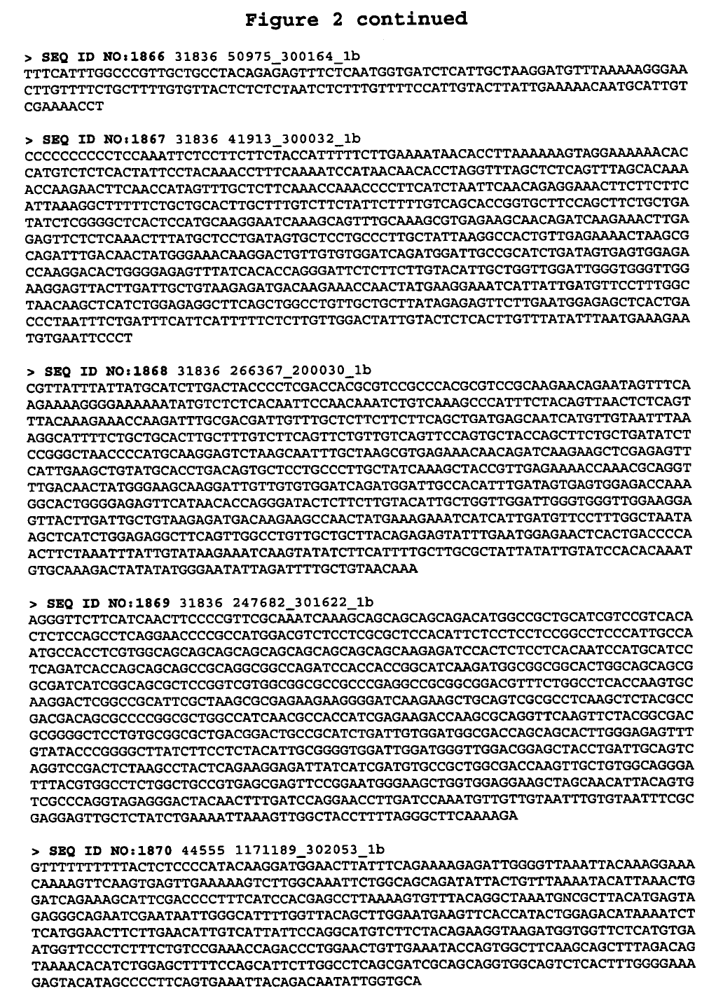

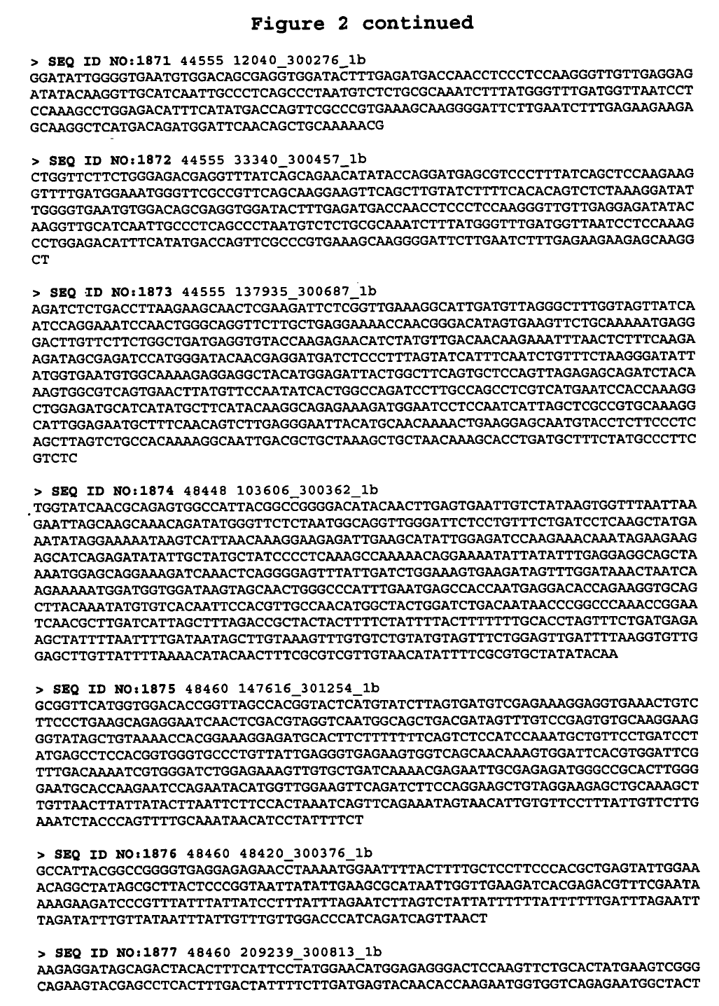

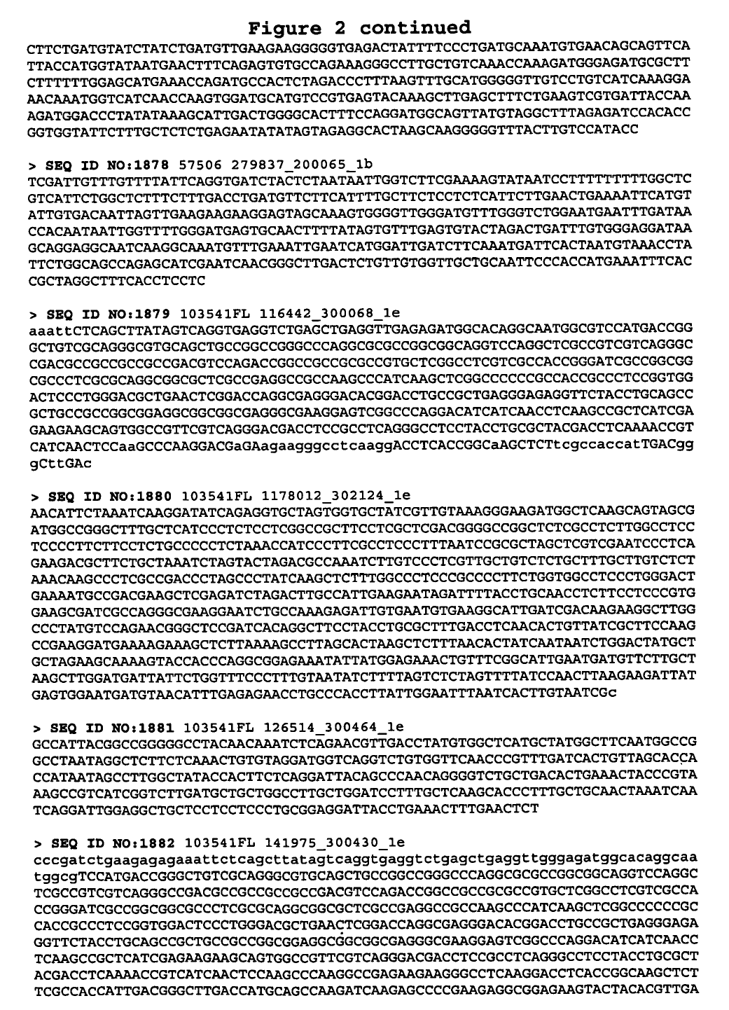

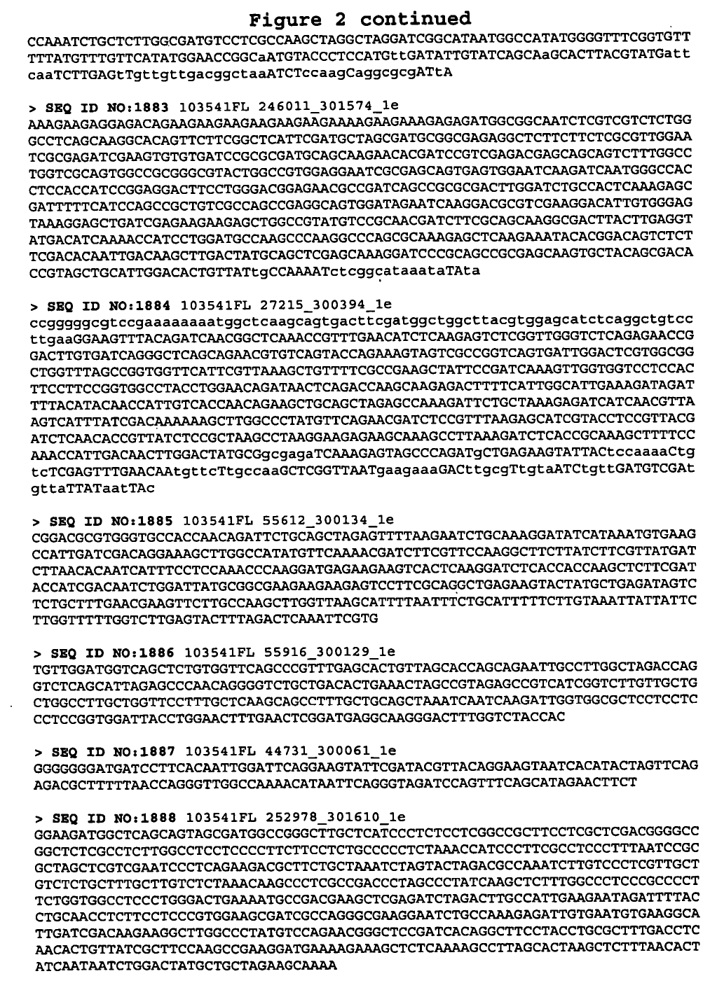

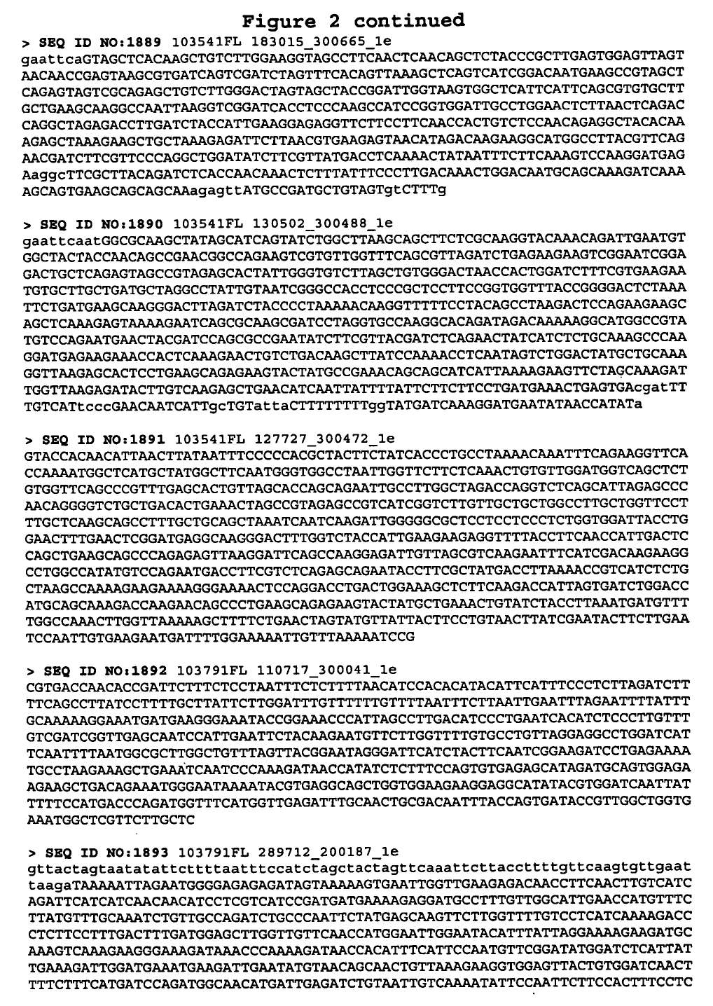

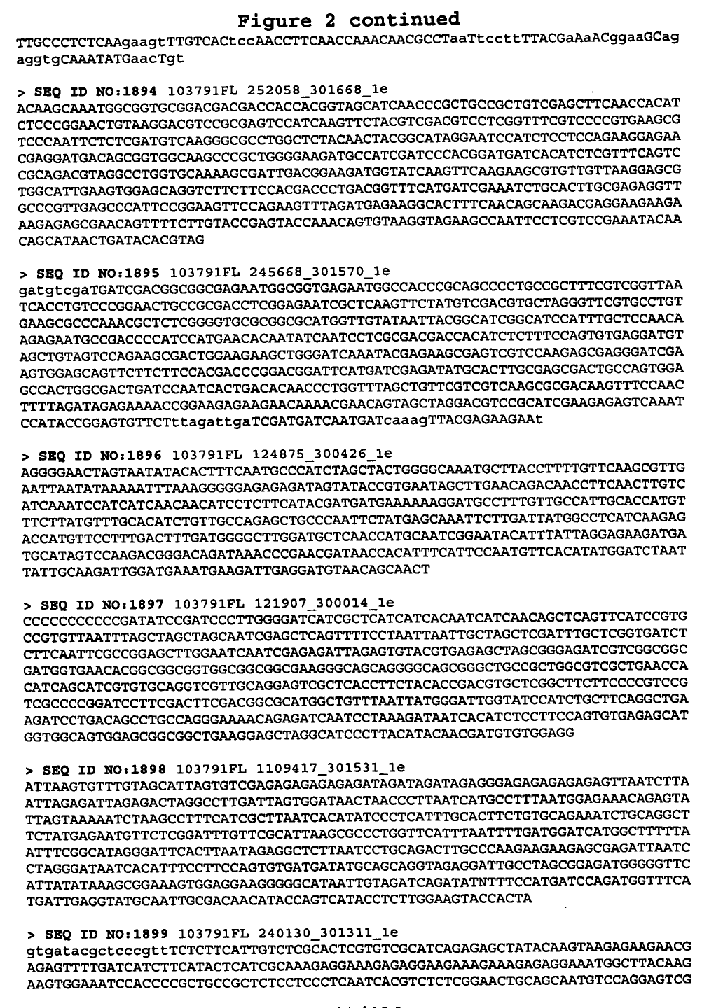

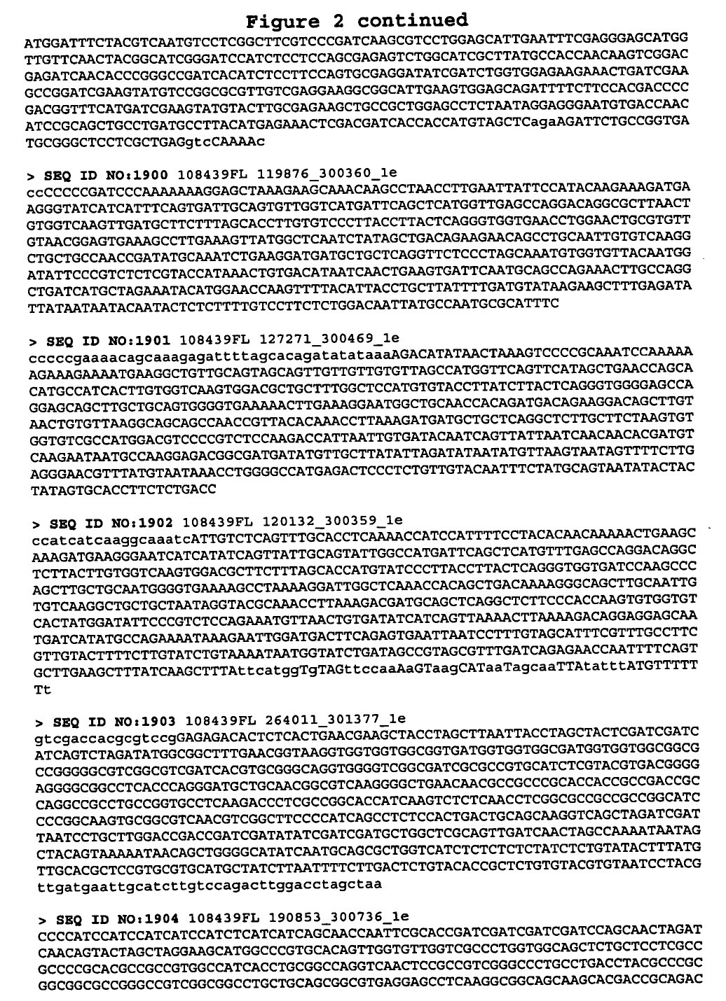

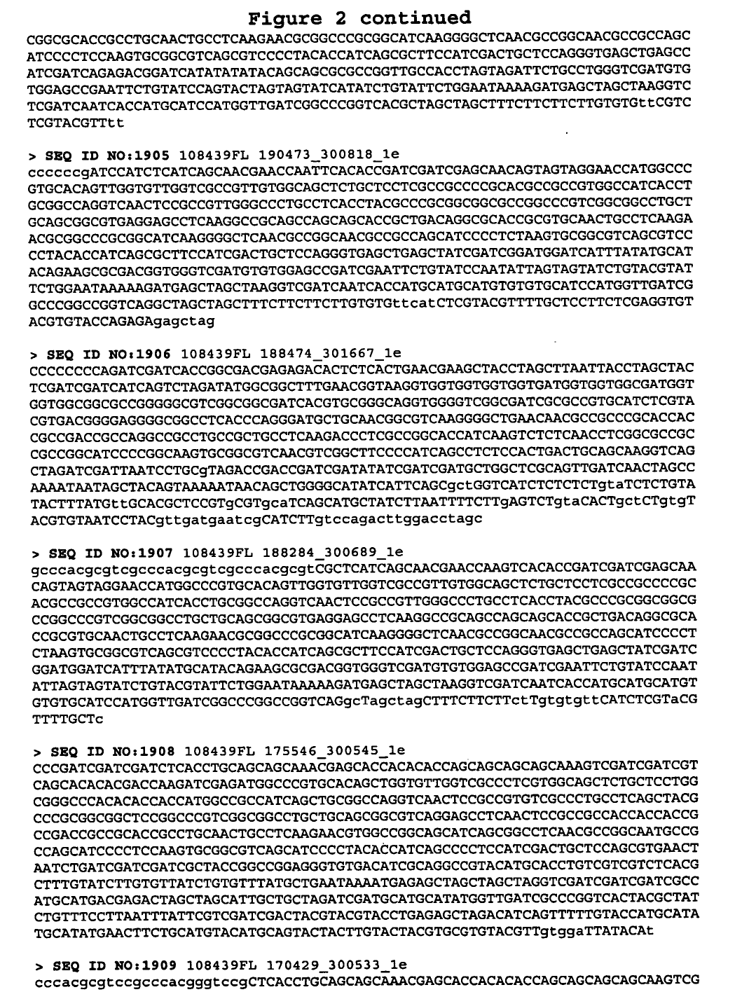

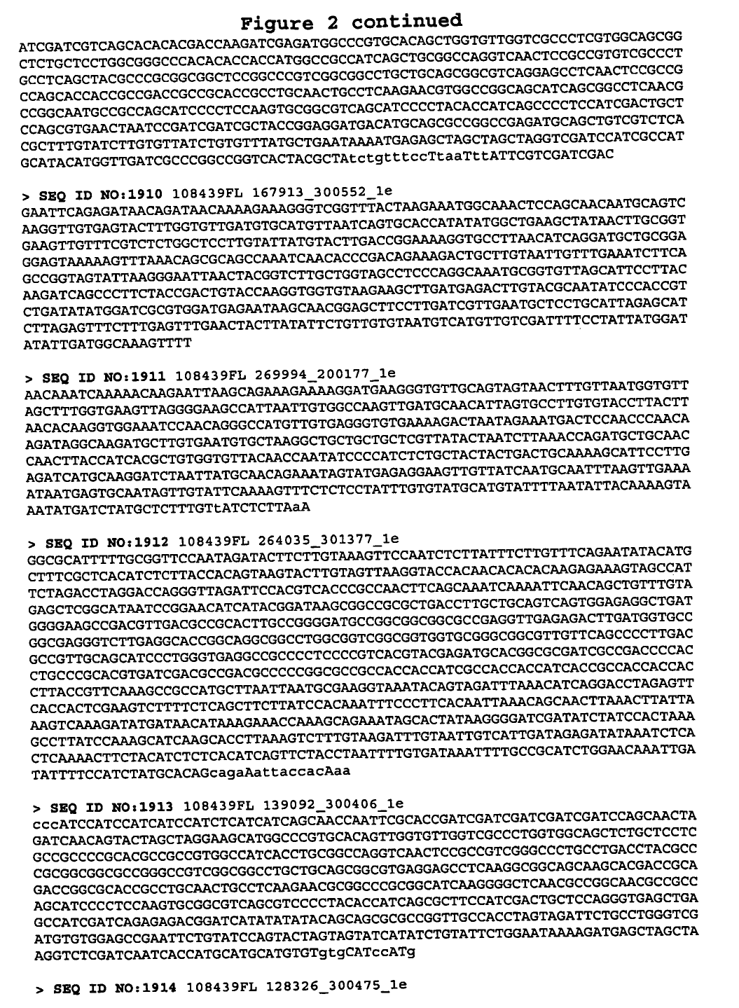

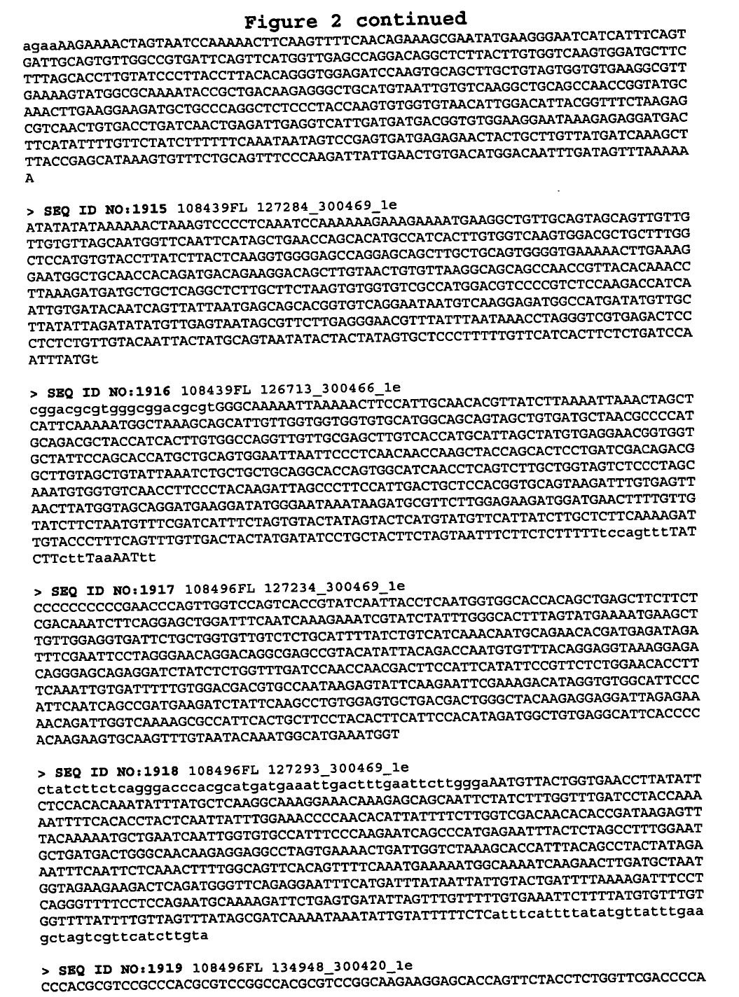

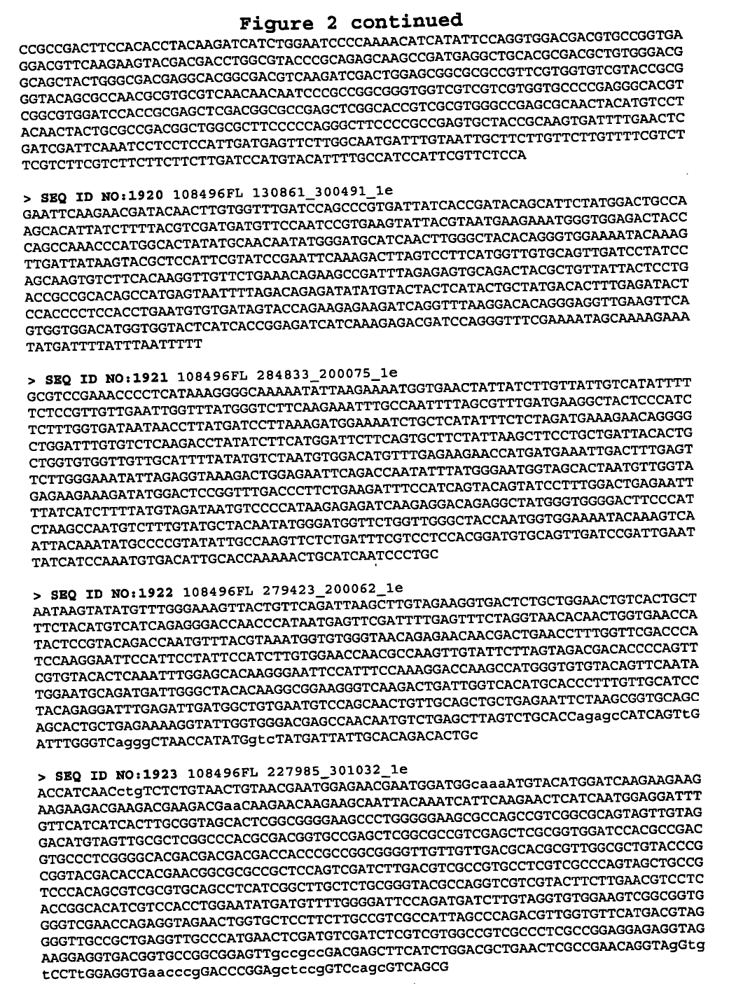

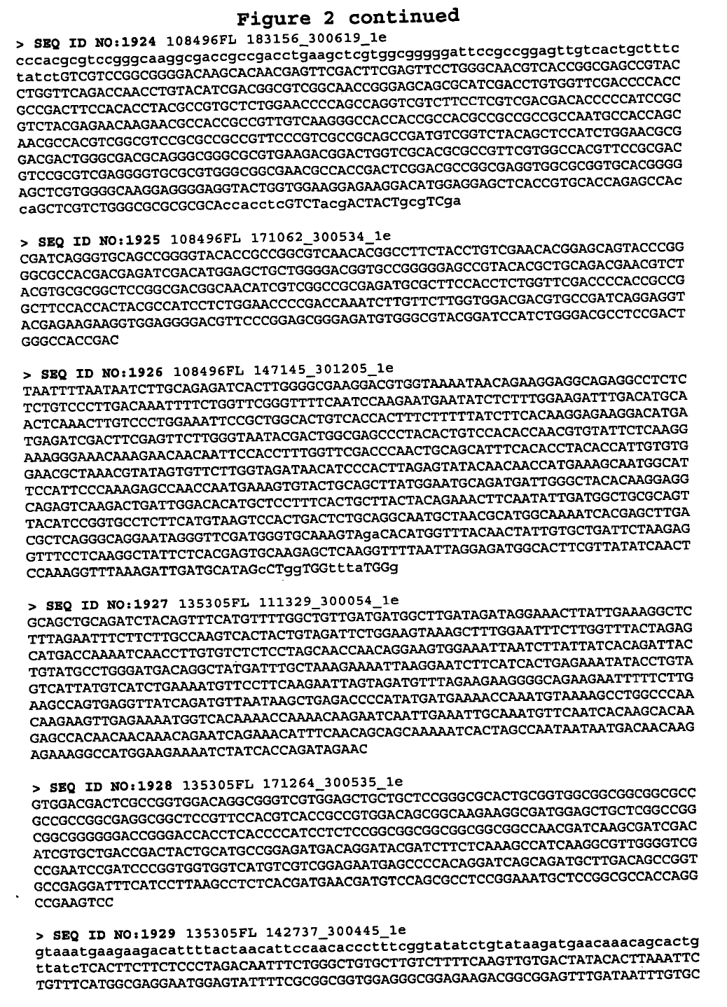

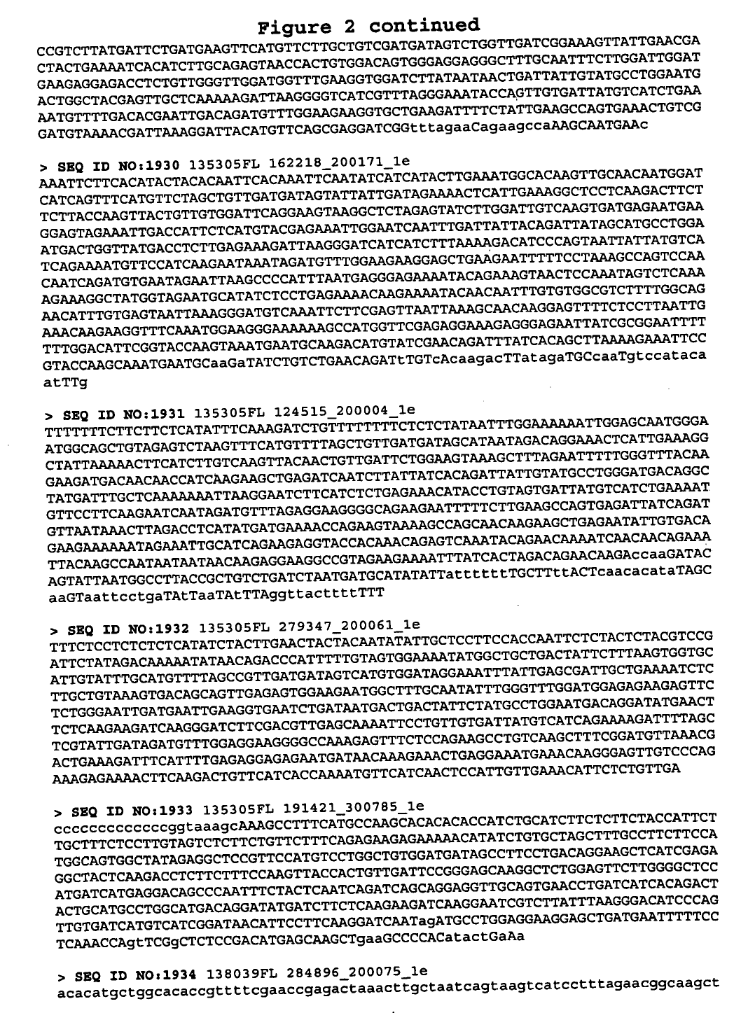

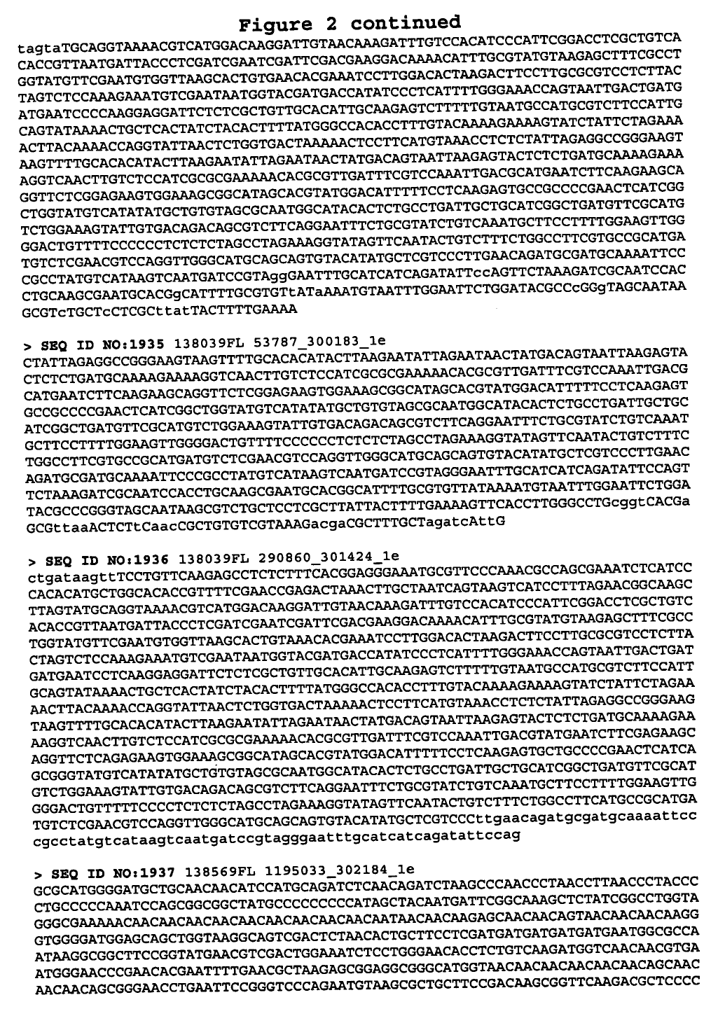

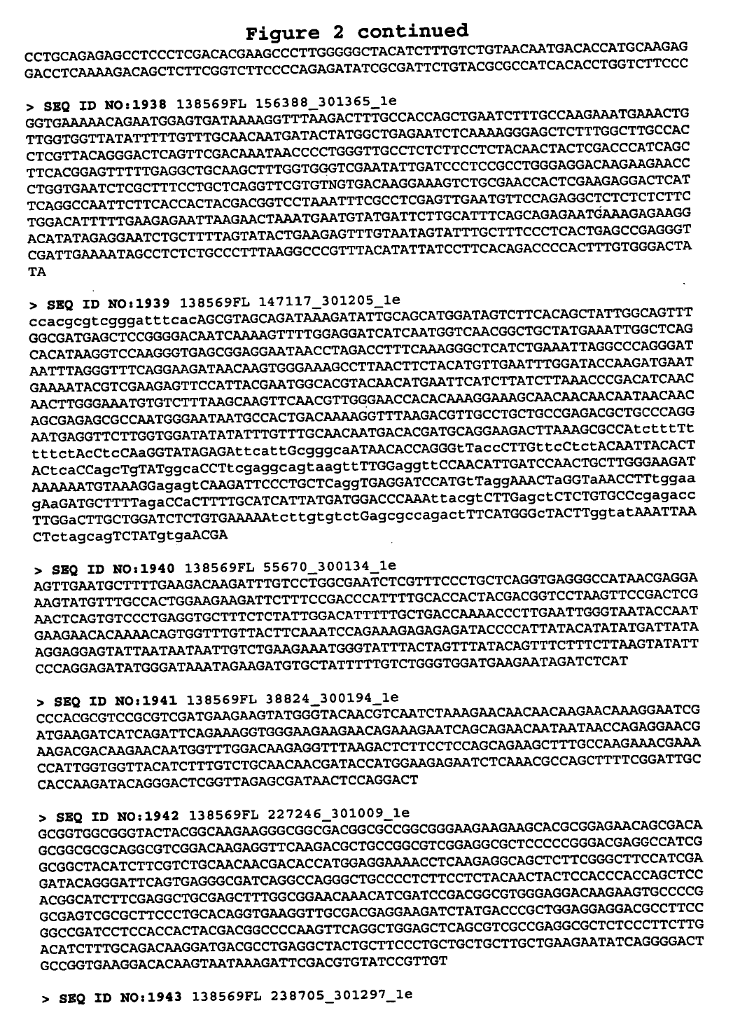

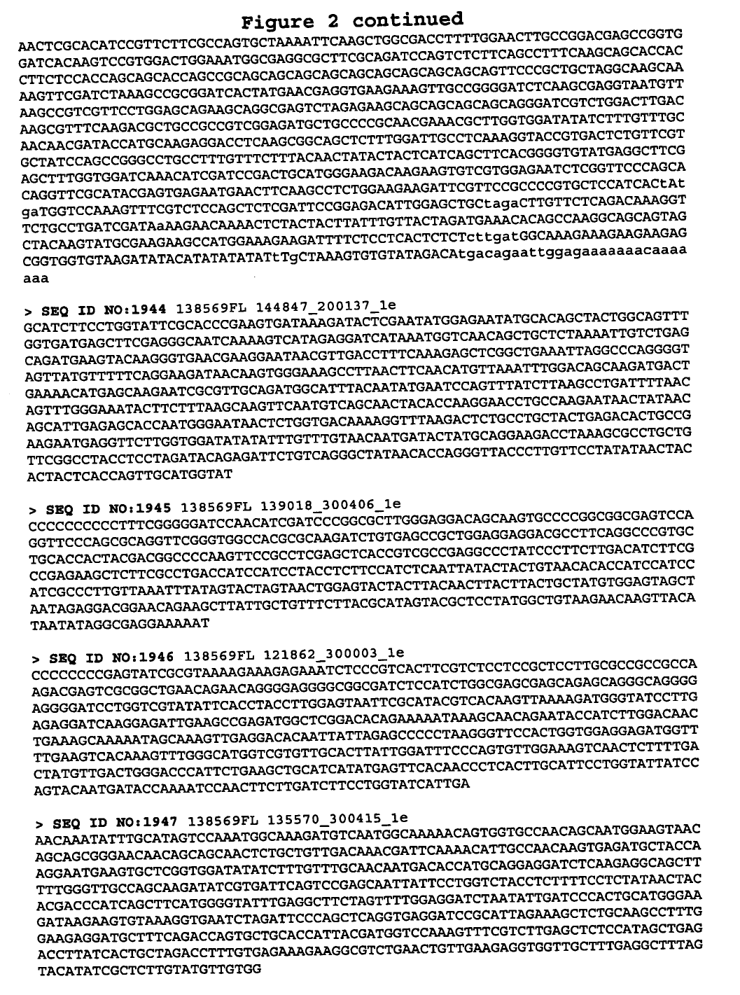

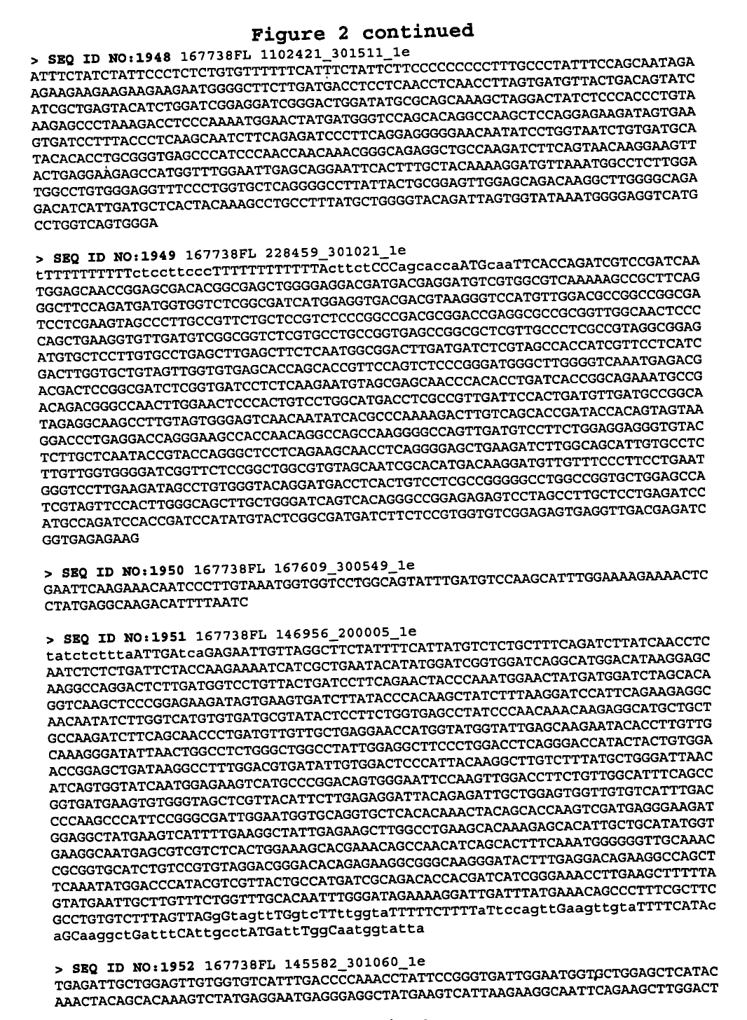

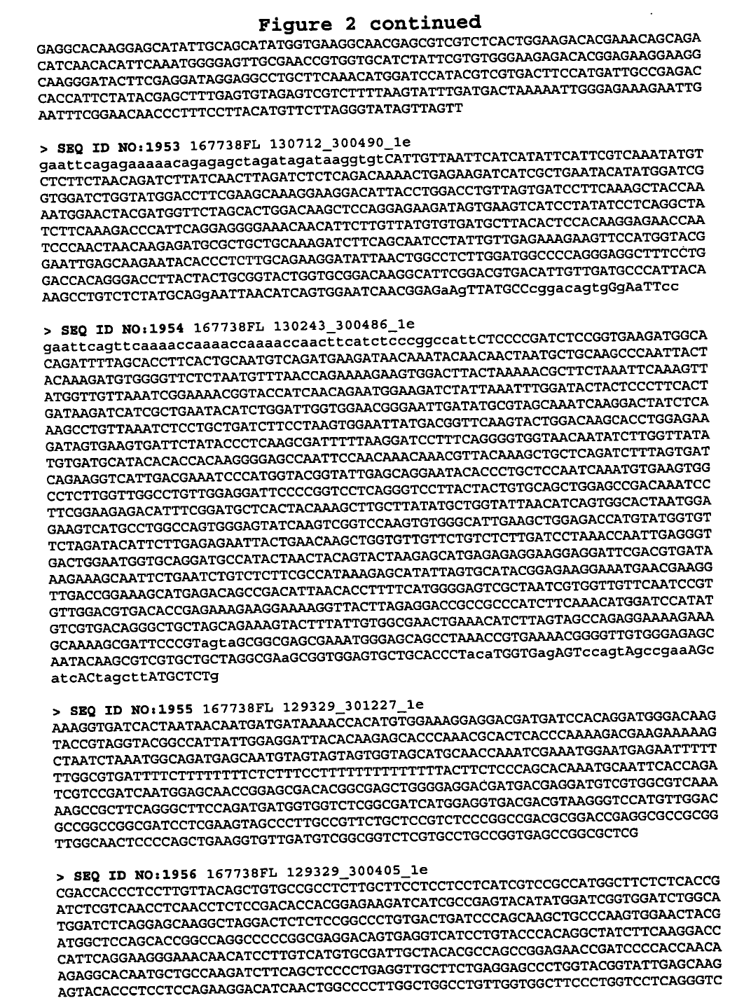

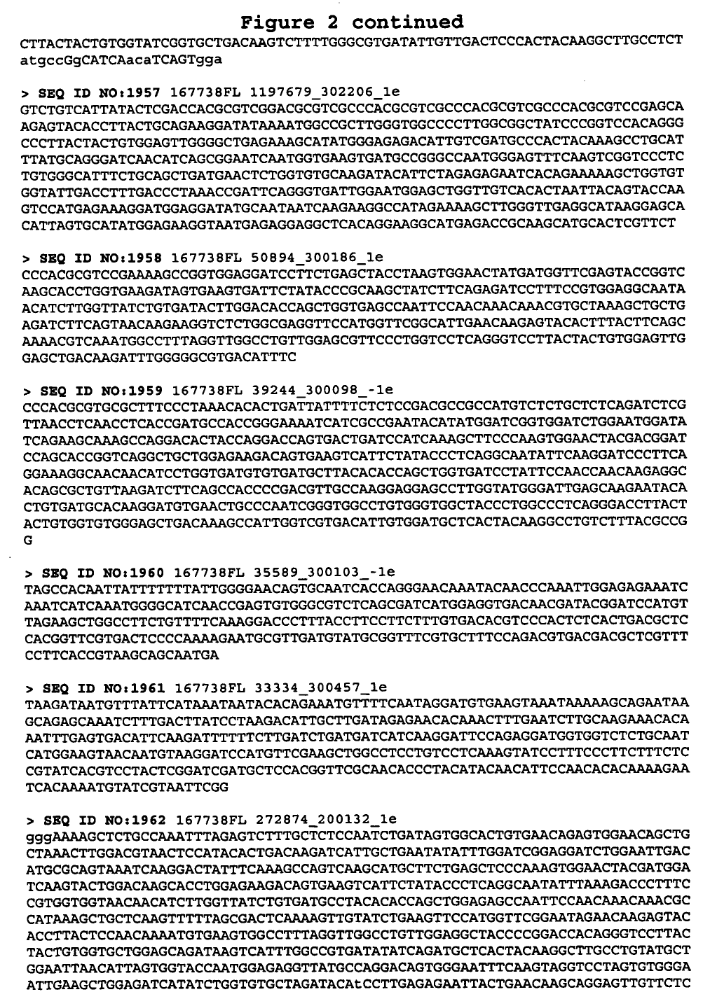

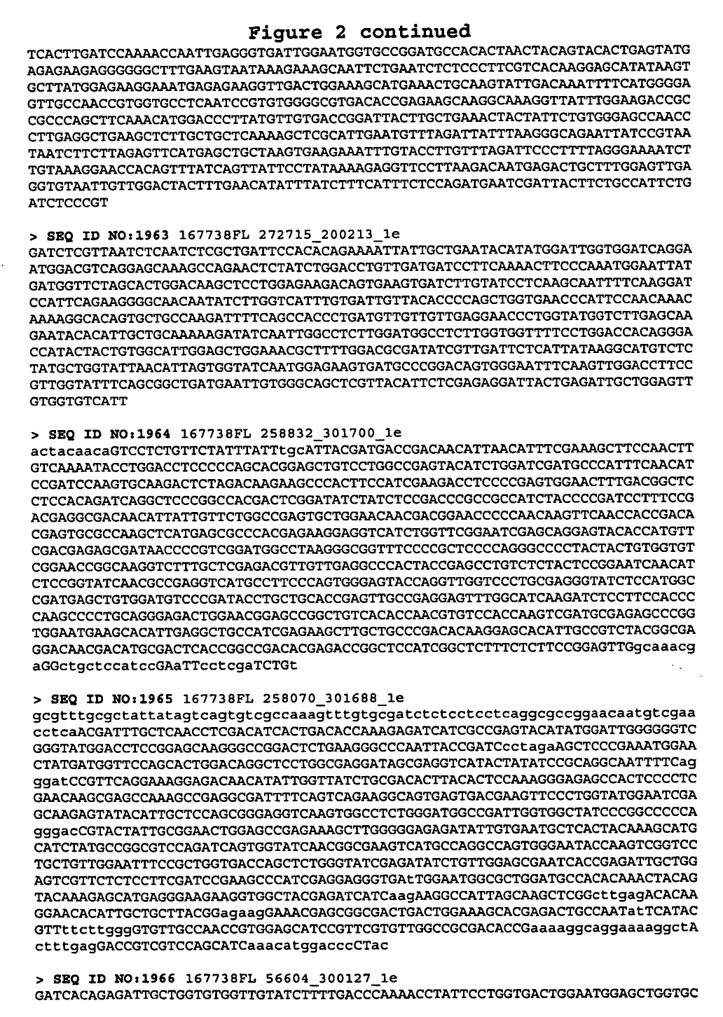

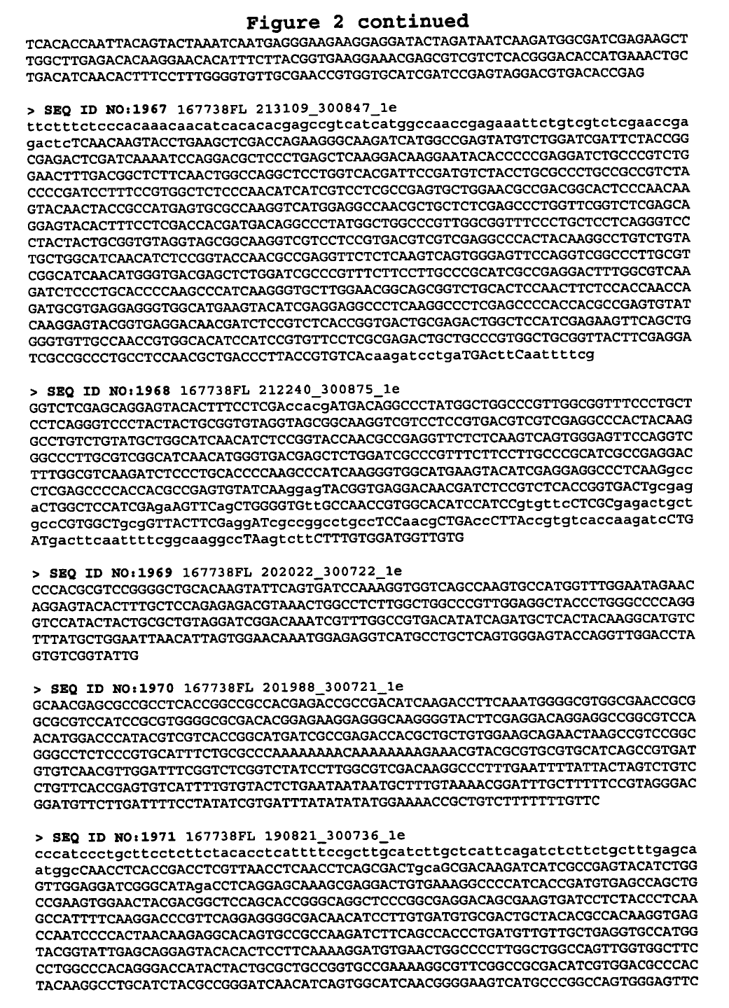

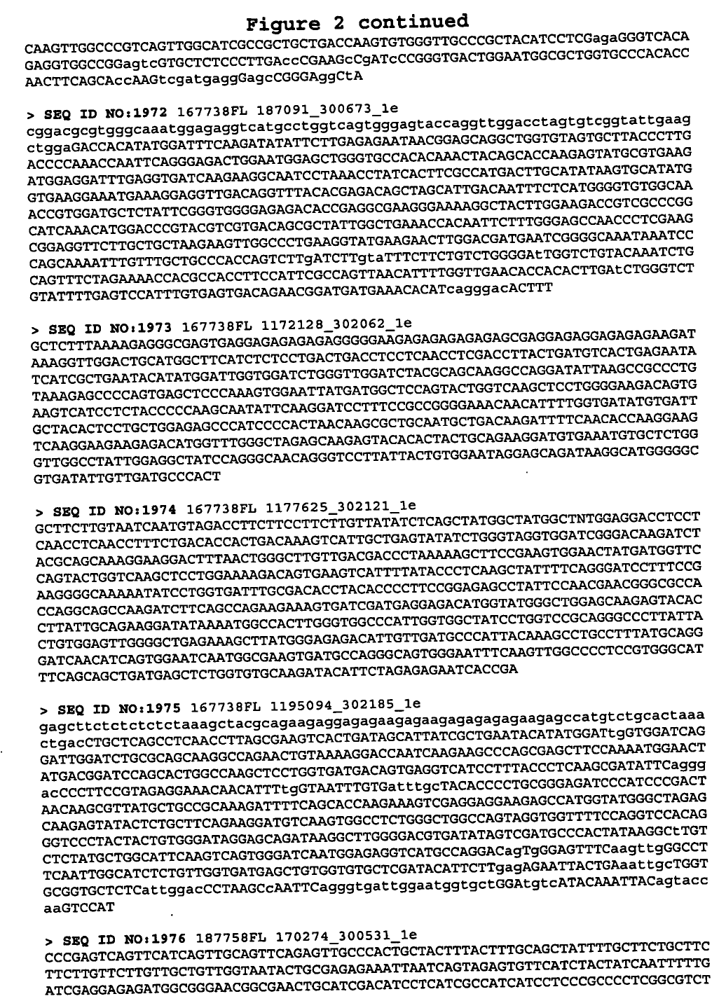

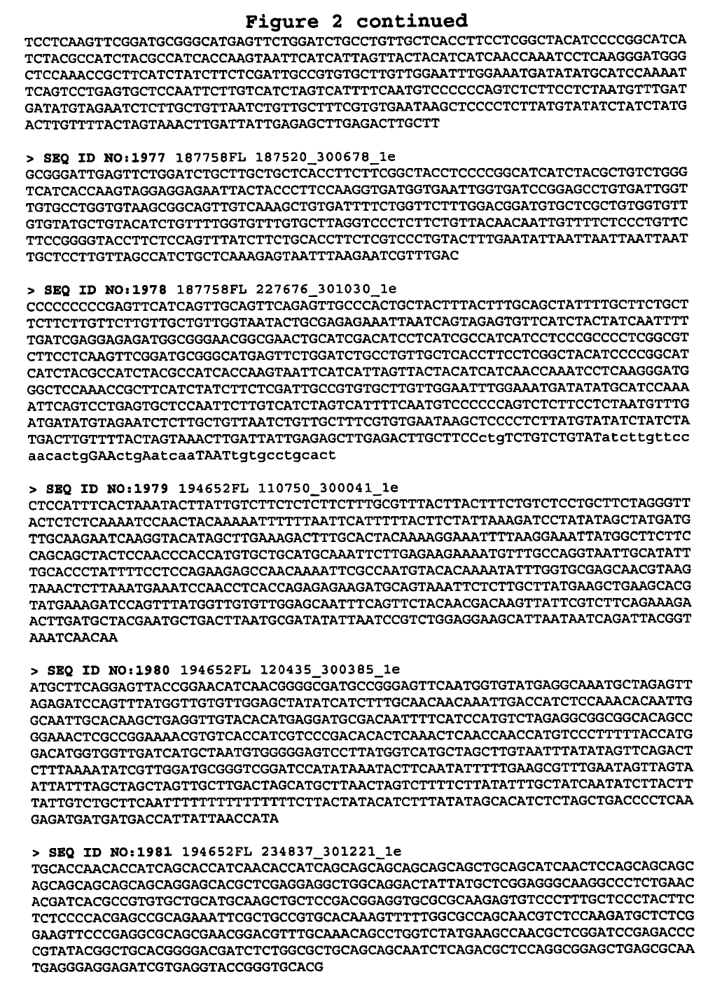

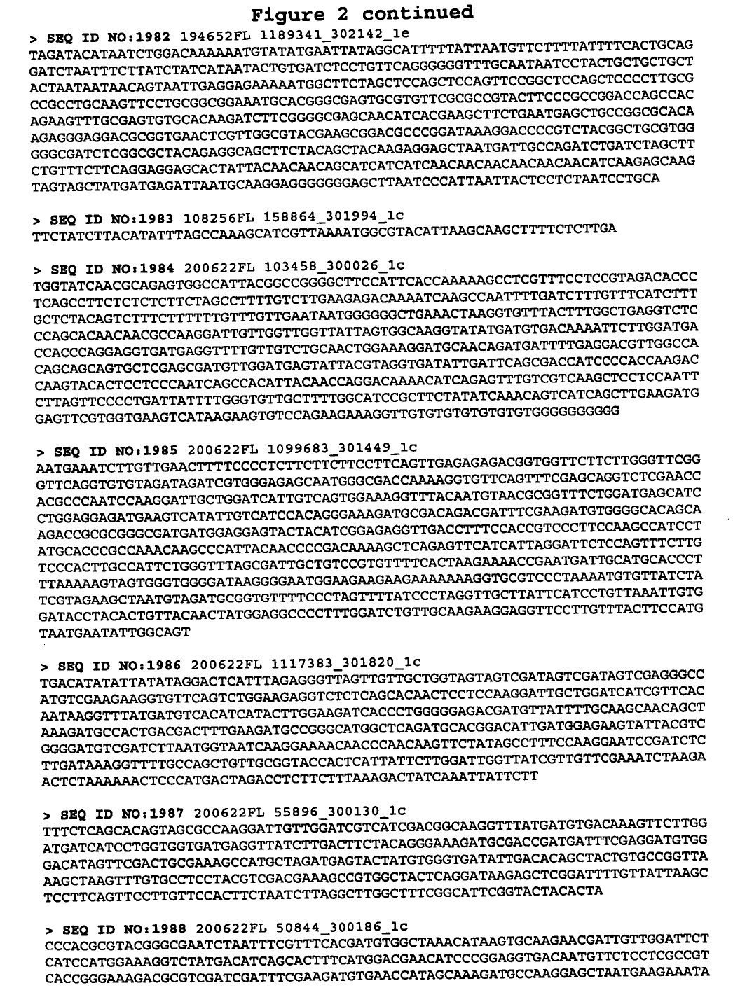

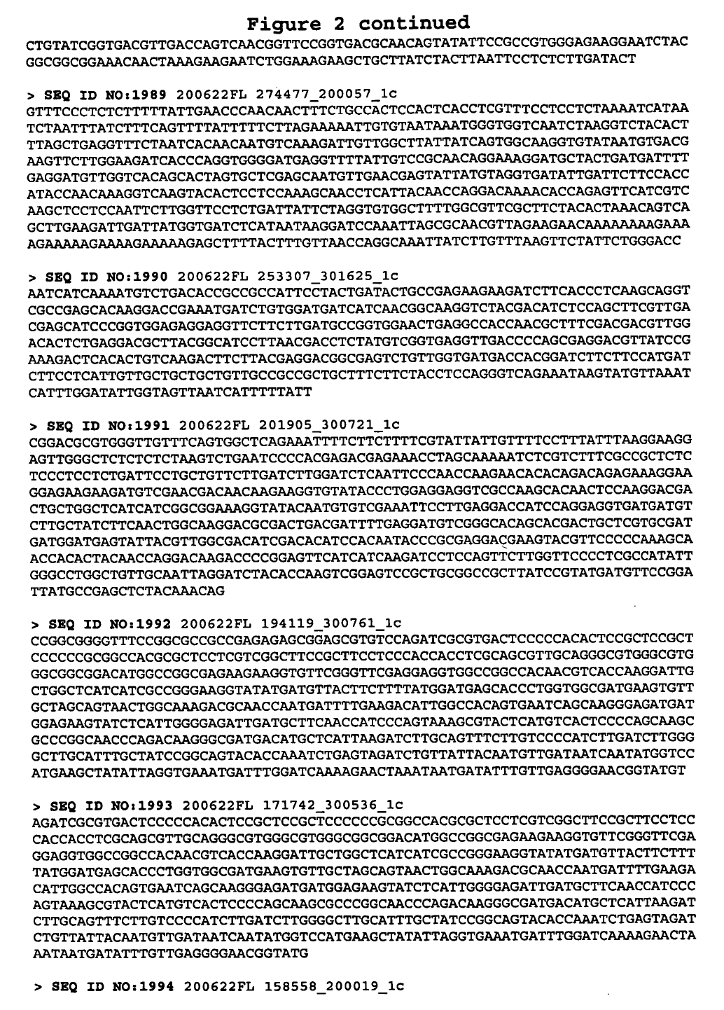

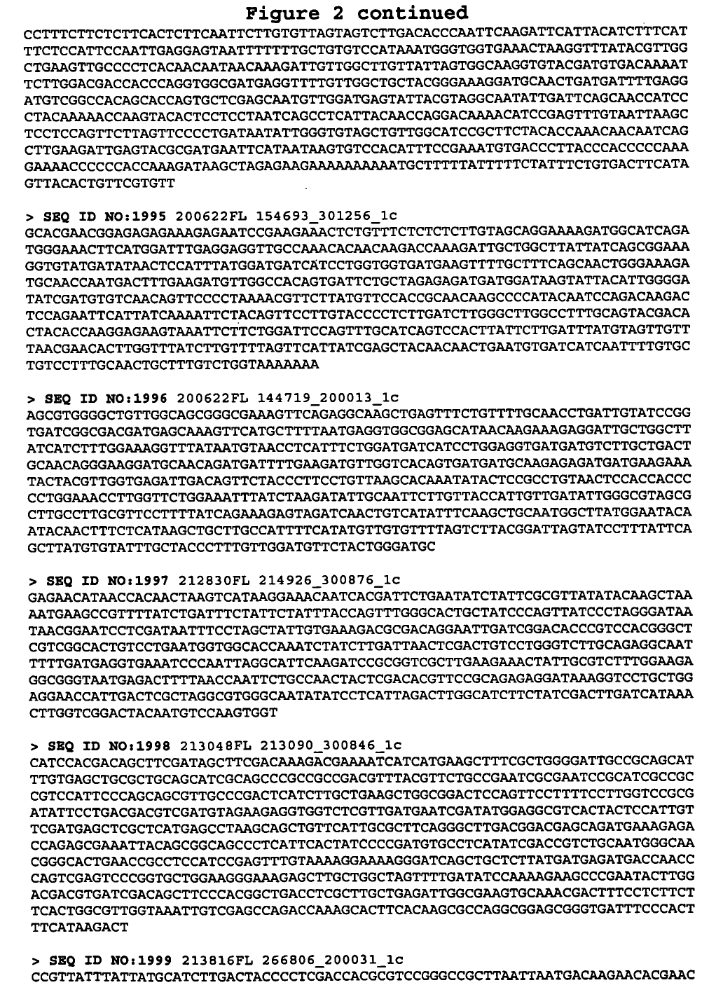

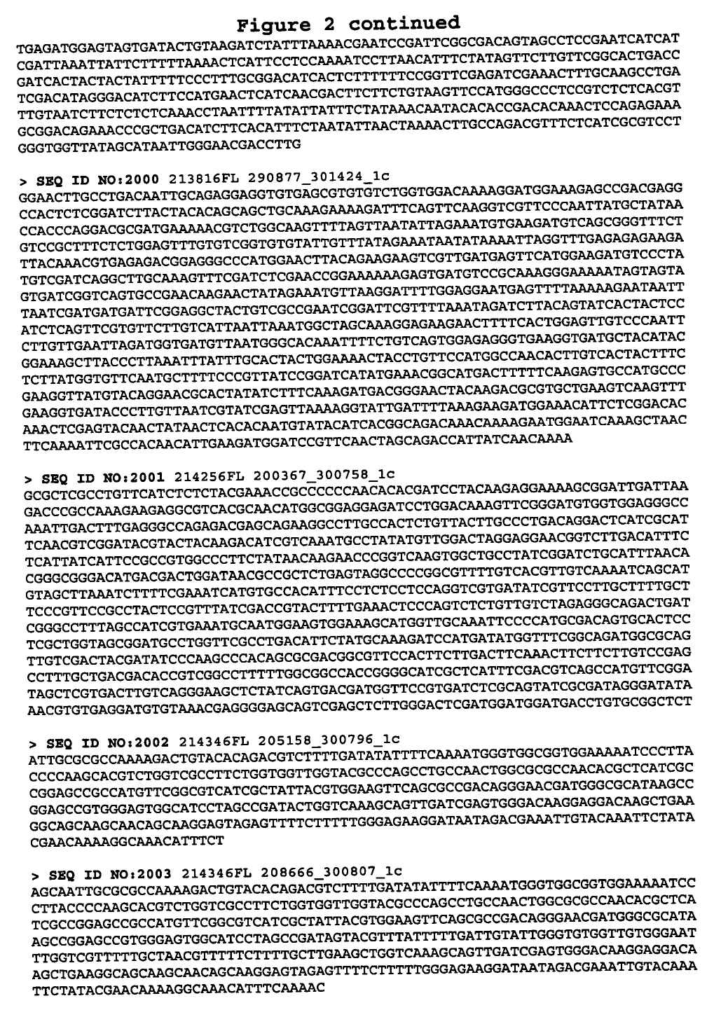

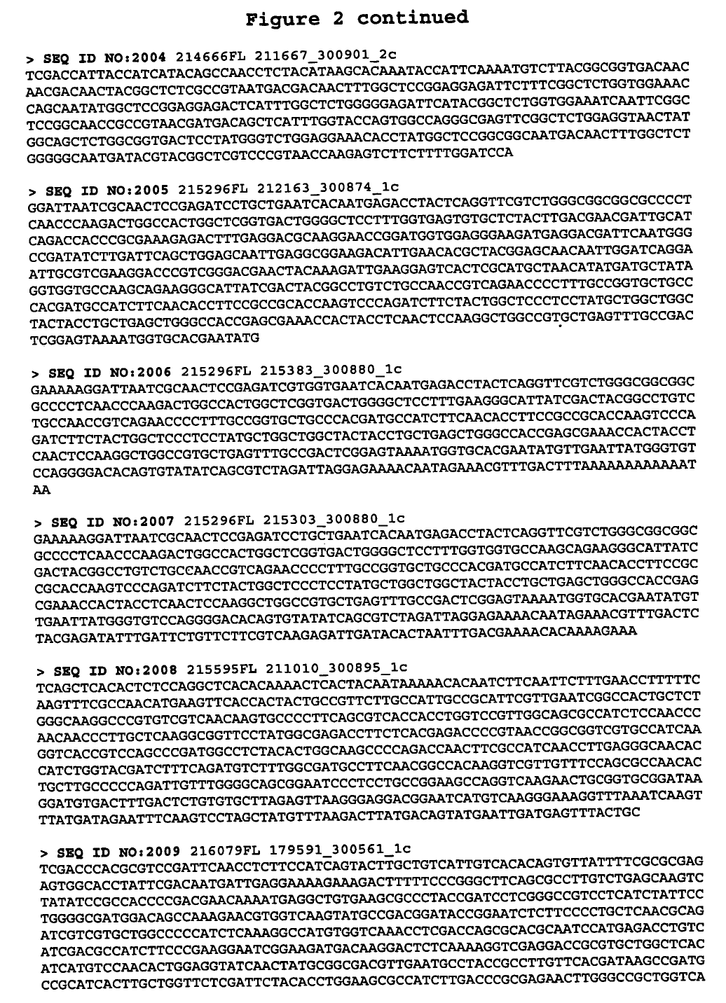

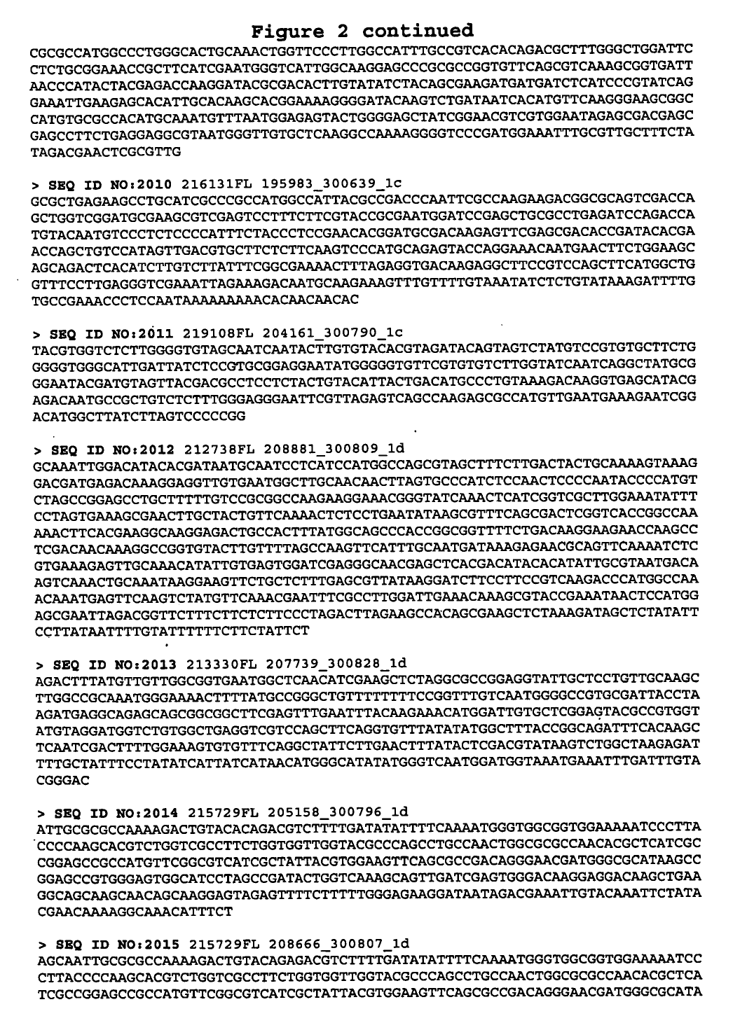

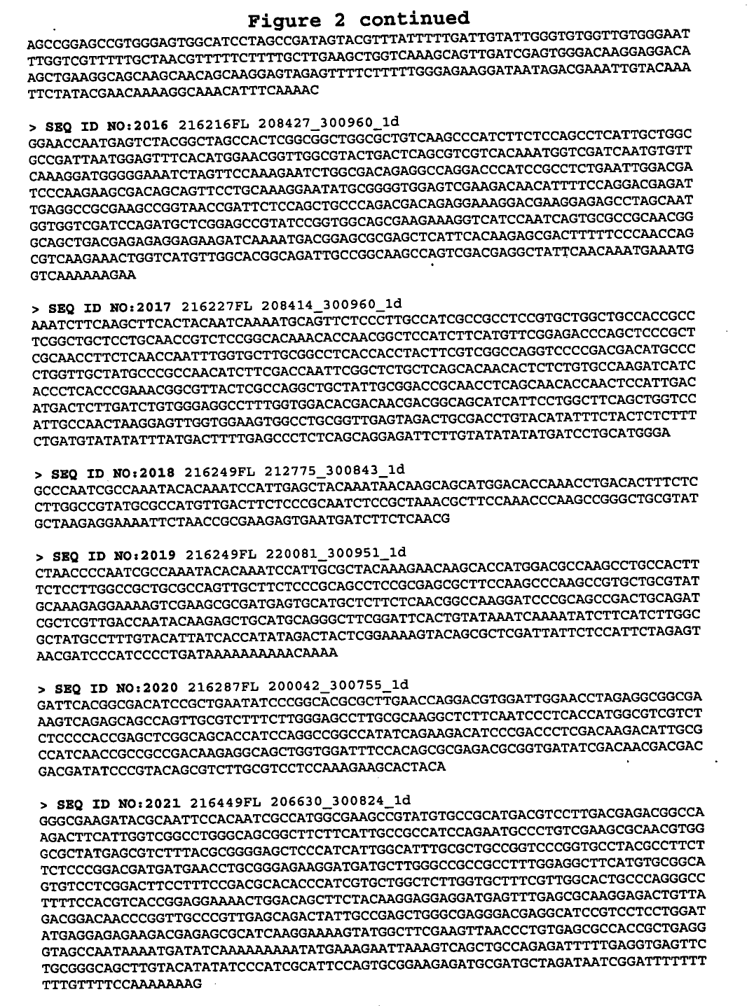

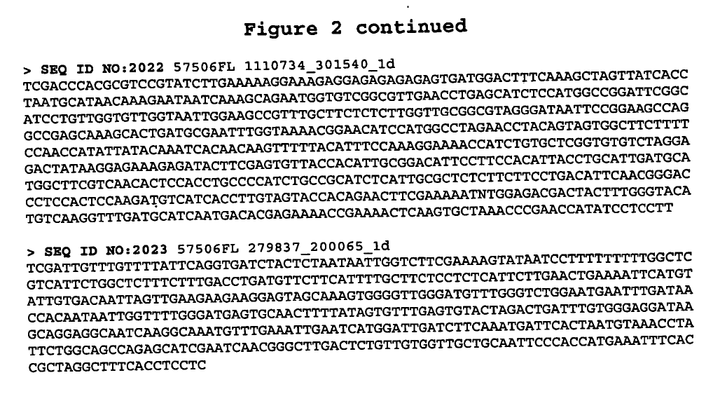

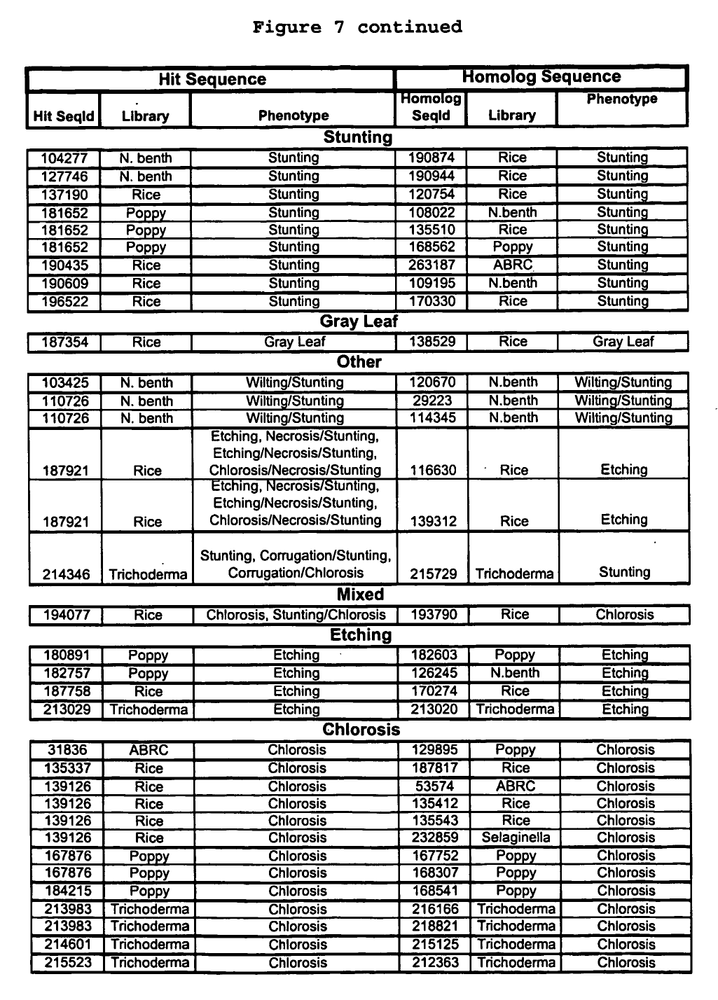

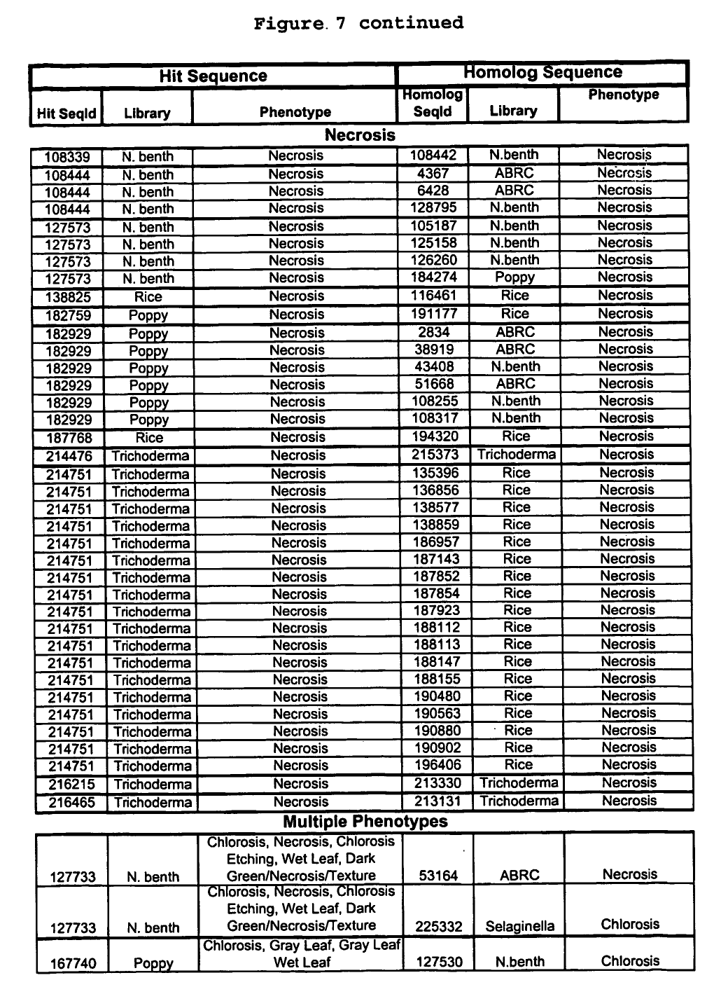

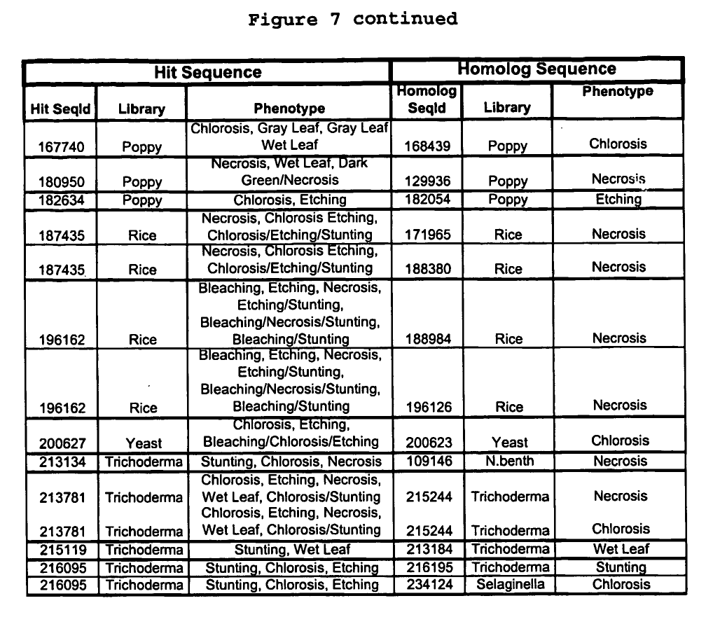

In some embodiments, the present invention comprises homologous nucleic acid sequences (SEQ ID NOs:312-2023) identified by screening an internal database with SEQ ID NOs.1-311 and 2024-2065 at a confidence level of Pz<1.00E-20. These sequences are provided in FIG. 2. The headers list the sequence identifier for the sequence that produced the actual phenotypic hit first and the sequence identifier for the homologous contig second. FIG. 7 contains altered visual phenotype data from representative homologs.

As will be understood by those skilled in the art, the present invention is not limited to the particular sequences of the homologs described above. Indeed, the present invention encompasses portions, fragments, and variants of the homologs as described above. Such variants, portions, and fragments can be produced and identified as described in Section III above. In particularly preferred embodiments, the present invention provides sequences that hybridize to SEQ ID NOs: 312-2023 under conditions ranging from low to high stringency. In other preferred embodiments, the present invention provides nucleic acid sequences that inhibit the binding of SEQ ID NOs:312-2023 to their complements under conditions ranging from low to high stringency. Furthermore, as described above in Section IV, the homologs can be incorporated into vectors for expression in a variety of hosts, including transgenic plants.

### EXAMPLES

### Example 1

### ABRC Library Construction in GENEWARE® Expression Vectors

Expressed sequence tag (EST) clones were obtained from the Arabidopsis Biological Resource Center (ABRC; The Ohio State University, Columbus, Ohio 43210). These clones originated from Michigan State University (from the labs of Dr. Thomas Newman of the DOE Plant Research Laboratory and Dr. Chris Somerville, Carnegie Institution of Washington) and from the Centre National de la Recherche Scientifique Project (CNRS project; donated by the Groupement De Recherche 1003, Centre National de la Recherche Scientifique, Dr. Bernard Lescure and colleagues). The clones were derived from cDNA libraries isolated from various tissues of Arabidopsis thaliana var Columbia. A clone set of 11,982 clones was received as glycerol stocks arrayed in 96 well plates, each with an ABRC identifier and associated EST sequence.

An ORF finding algorithm was performed on the EST clone set to find potential full-length genes. Approximately 3,200 full-length genes were found and used to make GENEWARE® constructs in the sense orientation. Five thousand of the remaining clones (not full-length) were used to make GENEWARE® constructs in the antisense orientation.

Full-length clones used to make constructs in the sense orientation were grown and DNA was isolated using Qiagen (Qiagen Inc., Valencia, Calif. 91355) mini-preps (as described in DNA Preparation section). Each clone was digested with NotI and Sse 8387 eight base pair enzymes. The resultant fragments were individually isolated and then combined. The combined fragments were ligated into pGTN P/N vector (with polylinker extending from PstI to NotI-5′ to 3′). For each set of 96 original clones approximately 192 colonies were picked from the pooled GENEWARE® ligations, grown until confluent in deep-well 96-well plates, DNA prepped and sequenced. The ESTs matching the ABRC data was bioinformatically checked by BLAST and a list of missing clones was generated. Pools of clones found to be missing were prepared and subjected to the same process. The entire process resulted in greater than 3,000 full-length sense clones.

The negative sense clones were processed in the same manner, but ligated into pGTN N/P vector (with polylinker extending from NotI to PstI-5′ to 3′). For each set of 96 original clones approximately 192 colonies were picked from the pooled GENEWARE® ligations and DNA prepped. The DNA from the GENEWARE® ligations was subjected to RFLP analysis using TaqI 4 base cutter. Novel patterns were identified for each set. The RFLP method was applied and only applicable for comparison within a single ABRC plate. This procedure resulted in greater than 6,000 negative sense clones.

The identified clones were re-arrayed, transcribed, encapsidated and used to inoculate plants.

### Example 2

### Construction of Tissue-specific N. benthamiana cDNA Libraries

A. mRNA Isolation: Leaf, root, flower, meristem, and pathogen-challenged leaf cDNA libraries were constructed. Total RNA samples from 10.5 μg of the above tissues were isolated by TRIZOL reagent (Life Technologies, Rockville, Md.). The typical yield of total RNA was 1 mg PolyA⁺RNA and was purified from total RNA by DYNABEADS oligo (T)₂₅. Purified mRNA was quantified by UV absorbance at OD₂₆₀ The typical yield of mRNA was 2% of total RNA. The purity was also determined by the ratio of OD₂₆₀/OD₂₈₀. The integrity of the samples had OD values of 1.8-2.0.

B. cDNA Synthesis: cDNA was synthesized from mRNA using the SUPERSCRIPT plasmid system (Life Technologies, Rockville, Md.) with cloning sites of NotI at the 3′ end and SalI at the 5′ end. After fractionation through a gel column to eliminate adapter fragments and short sequences, cDNA was cloned into both GENEWARE® vector p1057 NP and phagemid vector PSPORT (Life Technologies, Rockville, Md.) in the multiple cloning region between Not1 and Xho1 sites. Over 20,000 recombinants were obtained for all of the tissue-specific libraries.

C. Library Analysis: The quality of the libraries was evaluated by checking the insert size and percentage from representative 24 clones. Overall, the average insert size was above 1 kb, and the recombinant percentage was >95%.

### Example 3

### Construction of Normalized N. benthamiana cDNA Library in GENEWARE® Vectors

A. cDNA synthesis. A pooled RNA source from the tissues described above was used to construct a normalized cDNA library. Total RNA samples were pooled in equal amounts first, then polyA+RNA was isolated by DYNABEADS oligo (dT)₂₅. The first strand cDNA was synthesized by the Smart III system (Clontech, Palo Alto, Calif.). During the synthesis, adapter sequences with Sfi1a and Sfi1b sites were introduced by the polyA priming at the 3′ end and 5′ end by the template switch mechanism (Clontech, Palo Alto, Calif.). Eight μg first strand cDNA was synthesized from 24 μg mRNA. The yield and size were determined by UV absorbance and agarose gel electrophoresis.

B. Construction of Genomic DNA driver. Genomic DNA driver was constructed by immobilizing biotinylated DNA fragments onto streptavidin-coated magnetic beads. Fifty μg genomic DNA was digested by EcoR1 and BamH1 followed by fill-in reaction using biotin-21-dUTP. The biotinylated fragments were denatured by boiling and immobilized onto DYNABEADS by the conjugation of streptavidin and biotin.

C. Normalization Procedure. Six μg of the first strand cDNA was hybridized to 1 μg of genomic DNA driver in 100 μl of hybridization buffer (6×SSC, 0.1% SDS, 1× Denhardt's buffer) for 48 hours at 65° C. with constant rotation. After hybridization, the cDNA bound on genomic DNA beads was washed 3 times by 20 μl×SSC/0.1% SDS at 65° C. for 15 min and one time by 0.1×SSC at room temperature. The cDNA bound to the beads was then eluted in 10 μl of fresh-made 0.1N NaOH from the beads and purified by using a QIAGEN DNA purification column (QIAGEN GmbH, Hilden, Germany), which yielded 110 ng of normalized cDNA fragments. The normalized first strand cDNA was converted to double strand cDNA in 4 cycles of PCR with Smart primers annealed to the 3′ and 5′ end adapter sequences.

D. Evaluation of normalization efficiency. Ninety-six non-redundant cDNA clones selected from a randomly sequenced pool of 500 clones of a previously constructed whole seedling library were used to construct a nylon array. One hundred ng of the normalized cDNA fragments vs. the non-normalized fragments were radioactively labeled by ³²P and hybridized to DNA array nylon filters. The hybridization images and intensity data were acquired by a PHOSPHORIMAGER (Amersham Pharmacia Biotech, Chicago, Ill.). Since the 96 clones on the nylon arrays represent different abundance classes of genes, the variance of hybridization intensity among these genes on the filter were measured by standard deviation before and after normalization. The results indicated that by using this type of normalization approach, a 1000-fold reduction in variance among this set of genes could be achieved.

E. Cloning of normalized cDNA into GENEWARE® vector. The normalized cDNA fragments were digested by Sfi1 endonuclease, which recognizes 8-bp sites with variable sequences in the middle 4 nucleotides. After size fractionation, the cDNA was ligated into GENEWARE® vector p1057 NP in antisense orientation and transformed into DH5α cells. Over 50,000 recombinants were obtained for this normalized library. The percentage of insert and size were evaluated by Sfi digestion of randomly picked 96 clones followed by electrophoresis on 1% of agarose gel. The average insert size was 1.5 kb, and the percentage of insert was 98% with vector only insertions of >2%.

F. Sequence analysis of normalized cDNA library. Two plates of 96 randomly picked clones have been sequenced from the 5′ end of cDNA inserts. One hundred ninety-two quality sequences were obtained after trimming of vector sequences and other standard quality checking and filtering procedure, and subjected to BLASTX search in DNA and protein databases. Over 40% of these sequences had no hit in the databases. Clustering analysis was conducted based on accession numbers of BLASTX matches among the 112 sequences that had hits in the databases. Only three genes (tumor-related protein, citrin, and rubit) appeared twice. All other members in this group appeared only once. This was a strong indication that this library is well-normalized. Sequence analysis also revealed that 68% of these 192 sequences had putative open reading frames using the ORF finder program (as described above), indicating possible full-length cDNA.

### Example 4

### Rice cDNA Library Construction in GENEWARE® Vectors

Oryzae sativa var. Indica IR-7 was grown in greenhouses under standard conditions (12/12 photoperiod, 29° C. daytime temp., 24° C. night temp.). The following types of tissue were harvested, immediately frozen on dry ice and stored at −80° C.: young leaves (20 days post sowing), mature leaves and panicles (122 days post sowing). Mature and immature root tissue (either 122 or 20 days post sowing) was harvested, rinsed in ddH₂O to remove soil, frozen on dry ice and stored at −80° C.

The following standard method (Life Technologies) was used for generation of cDNA and cloning. High quality total RNA was purified from target tissues using Trizol (LTI) reagent. mRNA was purified by binding to oligo (dT) and subsequent elution. Quality of mRNA samples is essential to cDNA library construction and was monitored spectrophotmetrically and via gel electrophoresis. 2-5 μg of mRNA was primed with an oligo (dT)-Not1 primer and cDNA was synthesized (no isotope was used in cDNA synthesis). Sal1 adaptors were ligated to the cDNA, which was then subjected to digestion with Not1. Restriction fragments were fractionated based on size and the first 10 fractions were measured for DNA quantity and quality. Fractions 6 to 9 were used for ligations. 100 ng of GENEWARE® vector was ligated to 20 ng synthesized cDNA. Following ligations, the mixtures were kept at −20° C. For transformation, 1 μl to 10 μl ligation reaction mixture was added to 100 μl of competent E. coli cells (strain DH5α) and transformed using the heat shock method. After transformation, 900 μl SOC medium was added to the culture and it was incubated at 37° C. for 60 minutes. Transformation reactions were plated out on 22×22 cm LB/Amp agar plates and incubated overnight at 37° C.

### Example 5

### Poppy cDNA Library Construction in GENEWARE® Vectors

A. Plant Growth. A wild population of Papaver rhoeas resistant to auxin 2,4-Dichlorophenoxyacetic acid (2,4-D) was identified from a location in Spain and seed was collected. The seed was germinated at DAS and yielded a morphologically heterogeneous population. Leaf shape varied from deeply to shallowly indented. Latex color in some individuals was pure white when freshly cut, slowly changing to light orange then brown. Latex in other individuals was bright yellow or orange and rapidly changed to dark brown upon exposure to air. A single plant (PR4) with the white latex phenotype was used to generate the library.

B. RNA extraction. Approximately 1.5 g of leaves and stems were collected and frozen on liquid nitrogen. The tissue was ground to a fine powder and transferred to a 50 mL conical polypropylene screw cap centrifuge tube. Ten mL of TRIZOL reagent (Life Technologies, Rockville, Md.) was added and vortexed at high speed for several minutes of short intervals until an aqueous mixture was attained. Two mL of chloroform was added and the suspension was again vortexed at high speed for several minutes. The tube was centrifuged 15 minutes at 3100 rpm in a tabletop centrifuge (GP Centrifuge, Beckman Coulter, Inc, Fullerton, Calif.) for resolution of the phases. The aqueous supernatant was then carefully transferred to diethylpyrocarbonate (DEPC)-treated 1.5 mL microtubes and total RNA was precipitated with 0.6 volumes of isopropanol. To facilitate precipitation, the solution was allowed to stand 10 minutes at room temperature after thorough mixing. Following centrifugation for 10 minutes at 8000 rpm in a microcentrifuge (model 5415C, Eppendorf AG, Hamburg), the pellet of total RNA was washed with 70% ethanol, briefly dried and resuspended in 200 μL DEPC-treated deionized water. A 10 μL aliquot was examined by non-denaturing agarose gel electrophoresis.

C. cDNA synthesis. To generate cDNA, approximately 50 μg of total RNA was primed with 250 pmole of first strand oligo (TAIL: 5′-GAG-GAT-GTT-AAT-TAA-GCG-GCC-GCT-GCA-G(T)₂₃-3′)(SEQ ID NO:2066) in a volume of 250 μL using 1000 units of Superscript reverse transcriptase (Life Technologies, Rockville, Md.) for 90 minutes at 42° C. Phenol extraction was performed by adding an equal volume of phenol:chloroform:isoamyl alcohol (25:24:1 v/v), vortexing thoroughly, and centrifuging 5 minutes at 14,000 rpm in an Eppendorf microfuge. The aqueous supernatant phase was transferred to a fresh microfuge tube and the first strand cDNA:mRNA hybrids were precipitated with ethanol by adding 0.1 volume of 3 M sodium acetate and 2 volumes of absolute ethanol. After 5 minutes at room temperature, the tube was centrifuged 15 minutes at 14,000 rpm. The pellet was washed with 80% ethanol, dried briefly and resuspended in 100 μL TE buffer (10 mM TrisCl, 1 mM EDTA, pH 8.0). After adding 10 μL Klenow buffer (RE buffer 2, Life Technologies, Rockville, Md.) and dNTPs (Life Technologies, Rockville, Md.) to a final concentration of 1 mM, second strand cDNA was generated by adding 10 units of Klenow enzyme (Life Technologies, Rockville, Md.), 2 units of RNase H (Life Technologies, Rockville, Md.) and incubating at 37° C. for 2 hrs. The buffer was adjusted with β-nicotinamide adenine dinucleotide β-NAD) by addition of E. coli ligase buffer (Life Technologies, Rockville, Md.) and adenosine triphosphate (ATP, Sigma Chemical Company, St. Louis, Mo.) added to a final concentration of 0.6 mM. Double stranded phosphorylated cDNA was generated by addition of 10 units of E. coli DNA ligase (Life Technologies, Rockville, Md.), 10 units of T4 polynucleotide kinase (Life Technologies, Rockville, Md.) and incubating for 20 minutes at ambient temperature.

The double stranded cDNA was isolated through phenol extraction and ethanol precipitation, as described above. The pellet was washed with 80% ethanol, dried briefly and resuspended in a minimal volume of TE. The resuspended pellet was ligated overnight at 16° C. with 50 pmole of kinased AP3-AP4 adapter (AP-3: 5′-GAT-CTT-AAT-TAA-GTC-GAC-GAA-TTC-3′/AP-4: 5′-GAA-TTC-GTCGAC-TTA-ATT-AA-3′)(SEQ ID NOs:2067-2068) and 2 units of T4 DNA ligase (Life Technologies, Rockville, Md.). Ligation products were amplified by 20 cycles of PCR using AP-3 primer and examined by agarose gel electrophoresis.

Expanded adapter-ligated cDNA was digested overnight at 37° C. with PacI and NotI restriction endonucleases. The GENEWARE® vector pBSG1056 (Large Scale Biology Corporation (LSBC), Vacaville, Calif.) was similarly treated. Digested cDNA and vector were electrophoresed a short distance through low-melting temperature agarose. After visualizing with ethidium bromide and excising the appropriate fraction(s), the fragments were then isolated by melting the agarose and quickly diluting 5:1 with TE buffer to keep from solidifying. The diluted fractions were mixed in the appropriate ratio (approximately 10:1 vector:insert ratio) and ligated overnight at 16° C. using T4 DNA ligase. Characterization of the ligation revealed an average insert size of 1.27 kb. The ligation was transferred to LSBC where large scale arraying was carried out. Random sequencing of nearly 100 clones indicated that about 40% of the inserts had full length open frames.

### Example 6

### Regulatory Factors cDNA Library Construction in GENEWARE® Vectors

Transcription factors represent a class of genes that regulate and control many aspects of plant physiology, including growth, development, metabolism and response to the environment. In order to analyze a collection of regulatory factor genes, the PCR-based methods described below were used to construct a library of such genes from Arabidopsis thaliana and Saccharomyces cerevisiae. In addition, clones containing genes corresponding to regulatory factors from N. benthamiana, Oryzae sativa and Papaver rhoeas were selected, based on cDNA sequence, from the libraries generated in GENEWARE® vectors as described above.

A. Regulatory Factor Gene Targeting. Publicly accessible databases of genome sequence include data on a wide range of organisms, from microbes to human. Many of these databases include annotation along with gene sequences that predict function of the genes based on either experimental data or homology to characterized genes. The MIPS (Munich Information Center for Protein Sequences) database contains sequence information and annotation for both Arabidopsis thaliana and Saccharomyces cerevisiae genomes. Based on this annotation, open reading frame sequences of predicted yeast and Arabidopsis transcription factors were downloaded from MIPS and used for PCR primer design.

B. PCR Primer Design

18-20 base pairs of nucleotide sequences at both ends of each downloaded ORF were extracted and used to design the gene-specific portion of individual primers. In addition, flanking sequence and restriction sites were added to the ends of primers as shown in the following example:

```
5′ primer
GCCTTAATTAACTGCAGC atgtcgggtcgtgaagatgaag SEQ ID NO:2069
        PacI -------------
                            PstI 5′ gene-specific sequence
3′ primer
TTGATATCTAGAGCGGCCGCTTA tcatgtttcatcatcgaaatcatca SEQ ID NO: 2070
      EcoRV NotI
                          ------------  3′ gene-specific sequence
                          XbaI
```

C. Arabidopsis and Yeast Template Preparation. Total RNA was isolated from flowers and apical meristems of the Arabidopsis ecotype Columbia using the Qiagen RNA-easy kit (Cat. no. 75162). mRNA was subsequently isolated from total RNA using the MACS mRNA isolation kit from Miltenyl Biotec (cat. no. 751-02). First strand cDNA was synthesized from 10 μg of mRNA in the presence of Superscript II reverse transcriptase (Gibco BRL cDNA synthesis kit; cat. no. 18248-013) and NotI primer (5′-GACTAGTTCTAGATCGCGAGCGGCCGCCC(T)₃₀VN-3′)(SEQ ID NO:2071). The second strand was synthesized based on the manufacturers instructions. This cDNA was diluted 1:5 prior to DNA amplification.

Since most yeast genes do not contain introns, genomic DNA was used directly as a template for PCR. Genomic DNA from S. cerevisiae S288C was obtained directly from Research Genetics (ResGen, an Invitrogen company, Huntsville Ala., catalog #40802).

D. PCR Amplification. 1 μl of template DNA was subjected to PCR using the Hi Fi Platinum (hot start) DNA polymerase (Gibco-BRL cat. no. 11304-011) and gene-specific primers for each ORF. Each 50 μl reaction contained: 5 μl 10× buffer, 1 μl of 10 mM dNTP, 2 μl of 50 mM MgSO4, 1 μl of template cDNA, 10 pmoles of each primer and 0.2 unit of Platinum Hi Fi DNA polymerase. PCR reactions were carried out in a MJ Research (Model PTC 200) thermal cycler programmed with the following conditions:

3 min at 95° C.

30 cycles [95° C. 30 sec., 50° C. 30 sec., 72° C. 3 min.]

72° C. 10 min.

Following PCR, reactions were stored at −20° C. until ready for ligation.

D. Subcloning ORFs into GENEWARE® Vectors. To minimize cost and the labor involved in cloning of individual ORF, PCR products containing different ORFs were cloned into the GENEWARE® vectors as pooled DNAs. 30-75 PCR products were pooled, digested with PacI and NotI and purified from an agarose gel. Purified DNA was subsequently ligated into the GENEWARE® vector (5PN-Cap digested with PacI and NotI). Single colonies were selected, grown and their DNA analyzed for the presence of insert. Inserts were gel purified and sequenced, and the sequence compared to the MIP protein database to confirm that they covered the complete ORF. Unique sequences representing various related genes were selected to cover different genes within a multi-gene family. The efficiency of pooled cloning ranged from 30-50% (i.e., 30-50 clones were identified from analysis of 100 pooled PCR products). Following sequence identification of the clones, PCR products that were not represented in the first round of cloning were subsequently pooled together and subjected to a second round.

### Example 7

### Other Libraries: Regulatory Gene Selection

For each of the cDNA libraries generated from N. benthamiana, Oryzae sativa and Papaver Rhoeas, a unigene set of clones was established. Following basic library construction, all DNA sequences were subjected to BLASTN analysis against each other. Sequences that showed perfect homology across a minimum of 50 base pairs were clustered together. At this level each cluster putatively represents a unique gene. The size of cluster varies depends on the size and complexity of sequence population (sequenced library). A cluster may have only one sequence member, or consist of hundreds of member sequences. The clone with 5′-most sequence in a cluster was then selected to represent the gene. A collection of all the 5′-most sequences or clones was established as the unigene set for that particular library. In the example illustrated below, 4 EST sequences were clustered, representing a putative gene. The EST Seq 1 contained the most sequence information toward the 5′-end, indicating that this clone had the longest insert relative to other cluster members. This process allows removal of redundant clones and selection of the longest and most-likely full-length clones for subsequent screens.

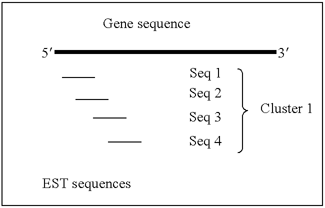

Based on the analysis of the sequence, and annotations of each unigene from each library, all clones that were homologous to known regulatory genes/transcription factors were targets for selection. Depending on the level of homology, some of the clones represented well characterized regulatory genes; however, many of the selected clones had only a modest level of homology to known genes or genes of very distantly related organisms. It is believed that this selection process can increase the probability of gene discovery, and by eliminating non-relevant clones, increase screen efficiency.

### Example 8

### Trichoderma cDNA Library Construction in GENEWARE® Vectors

A. Growth and Induction of Trichoderma harzianum rifai 1295-22. Cultures of Trichoderma harzianum rifai 1295-22 were obtained from ATCC (cat.# 20847) and propagated on PDA. Liquid cultures were inoculated and induced using a protocol derived from Vasseur et al. (Microbiology 141:767-774, 1995) and Cortes et al. (Mol. Gen. Genet. 260:218-225, 1998): agar-grown cells were used to inoculate a 100 ml culture in PDB and grown 48 hours at 29° C. with agitation. Mycelia were harvested by centrifugation, transferred to Minimal Media (MM) +0.2% glucose, and incubated overnight at 29° C. with agitation. Mycelia were harvested again by centrifugation, washed with MM, resuspended in MM and incubated 2 hours at 29° C. with agitation. Mycelia were harvested again by centrifugation, divided into 2 aliquots, and used to inoculate 1)125 ml MM +0.2% glucose or 2) 125 ml MM +1 mg/ml elicitor. Elicitor is a preparation of cell walls from Rhizoctonia solani grown in liquid culture and isolated according to Goldman et al. (Mol. Gen. Genet. 234:481-488, 1992). Induced and uninduced cultures were incubated at 29° C. with agitation, harvested after 24 and 48 hours by filtration and immediately frozen in liquid nitrogen. Aliquots were assayed for induction using 2-D gel SDS-PAGE to compare induced and uninduced cultures. Both induced and uninduced (24 hours) tissue was used for subsequent RNA isolation and library construction.

B. RNA Isolation and Library Construction. mRNA isolation was accomplished by magnetically labeling polyA⁺ RNA with oligo (dT) microbeads and selecting the magnetically labeled RNA over a column. The purified polyA⁺ RNA was then used for cDNA synthesis using a modified version of the full-length enrichment reactions (cap-capture method) described by Seki et al. (Plant J. 15:707-720, 1998). Specifically, isolated mRNA was primed with NotI-oligo d(T) primer to synthesize the first strand cDNA. After the synthesis reaction, a biotin group was chemically introduced to the diol residue of the cap structure of the mRNA molecule. RNase I treatment was then used to digest the mRNA/cDNA hybrids, followed by binding of streptavidin magnetic beads. After this step, the full-length cDNAs were then removed from the beads by RNaseH and tailed with oligo dG by terminal transferase or used directly in the 2^{nd} strand synthesis. For the oligo dG tailed samples, the second strand cDNAs were then synthesized with PacI-oligo dC primers and DNA polymerase. Additional modifications to the published procedure include: addition of trehalose and BSA as enzyme stabilizers in the reverse transcriptase reaction, a temperature of 50 to 60° C. for the first strand cDNA synthesis reaction, high stringency binding and washing conditions for capturing biotinylated cap-RNA/cDNA hybrids and substitution of the cDNA poly (dG) tailing step with a Sal-I linker ligation.

The cDNA was size-fractionated over a column and the largest 2-3 fractions were collected and used to ligate with GENEWARE® vector pBSG1057. The ligation reaction was transformed into E. coli DH5α and plated, the transformation efficiency was calculated and the DNA from the transformants was subjected to the quality control steps described below:

1. cDNA synthesis/cloning: The cloning efficiency must be greater than 8×105 cfu/μg.

2. Restriction enzyme digestion and sequencing: 500 to 1,000 transformants were picked and DNA isolated. cDNA inserts were digested out by appropriate restriction enzymes and checked by gel electrophoresis. The average insert size was calculated from 100 random clones. If the average size was >0.9 kbp, the DNA preps were then passed on to the sequencing group to obtain 5′-end sequences. Those sequences were used to further evaluate the of the library. Libraries that did not meet QC standards, such as high vector background (>5%), low full-length percentage (<60%), or short average insert size (<0.7 kbp), were discarded, and the entire procedure repeated.

C. Library Subtraction. The induced Trichoderma library in GENEWARE® was constructed as above and a large number of clones were arrayed on a nylon membrane at high density (HD array). Based on the genomic size and expression levels of S. cerevisiae, 18,000 colonies were imprinted to provide 3-fold coverage of the expressed genes. Freshly grown colonies were plated out and picked into 384 well plates and then imprinted on Nylon membranes in 3×3 format at duplicated locations. First strand cDNAs to use as probes were synthesized from mRNAs isolated from both induced and uninduced tissue and used to hybridize the HD arrays. The intensity of each clone after hybridization was quantitated by phosphoimage scanning. The locations of all 18,000 spots were tracked by Array Vision software, which also determined the local background and calculated the signal/noise ratio for every clone on the membrane. The data generated were then converted to Excel format and analyzed to obtain the fold of induction or down-regulation. Based on the measured noise level, a 5-fold increase or decrease, relative to controls, was used as a cutoff value. Clones displaying ≧5-fold induction or reduction on duplicated samples were chosen. These clones were robotically re-arrayed using a Qbot device (see below, Colony Array) DNA was prepped as described below and sequenced. Based on the clustering results, 5′-most unigenes were selected and rearrayed using the procedures described for the Poppy library above: the total number of clones that were selected was 1,019 for the up-regulated library (Th03), and 851 for the down-regulated library (Th04). These clones were prepared as described below (DNA Preparation, Transcription, Inoculation) and tested in a functional genomic screens for altered visual phenotypes.

### Example 9

### Colony Array

A. Colony Array—Picking. Ligations were transformed into E. coli DH5α cells and plated onto 22×22 cm Genetix “Q Trays” prepared with 200 ml agar, Amp¹⁰⁰. A Qbot device (Genetix, Inc., Christchurch, Dorset UK) fitted with a 96 pin picking head was used to pick and transfer desired colonies into 384-well plates according to the manufacturers specifications and picking program SB384.SC1, with the following parameters:

### Source

Container: Genetix bioassay tray

Color: White

Agar Volume: 200 ml

### Destination

Container: Hotel (9 High)

Plate: Genetix 384 well plate

Time In Wells (sec): 2

Max Plates to use: # of 384 well plates

1^{st} Plate: 1

Dips to Inoculate: 10

Well Offset: 1

### Head

Head: 96 Pin Picking Head

First Picking Pin: 1

Pin Order: A1-H1, H2-A2 . . . (snaking)

### Sterilizing

Qbot Bath #1

Bath Cycles: 4

Seconds in Dryer: 10

Wait After Drying: 10

(approximate picking time: 8 hrs/20,000 colonies)

Following picking, 384 well plates containing bacterial inoculum were grown in a HiGro chamber fitted with O₂ at 30° C., speed 6.5 for 12-14 hours. Following growth, plates were replicated using the Qbot with the following parameters, 2 replication runs per plate:

Source

Container: Hotel (9 High)

Plate: Genetix Plate 384 Well

Plates to replicate: 24

Start plate No.: 1

No. of copies: 1

Destination

Container: Universal Dest Plate Holder

Plate: Genetix Plate 384 Well

No. of Dips: 5

Head

Head: 384 Pin Gravity Gridding Head

Sterilizing

Qbot Bath #1

Bath cycles: 4

Seconds in Dryer: 10

Wait After Drying: 10

Airpore tape was placed over the replicated 384 well plates and the replicated plates were grown in the HiGro as above for 18-20 hours, sealed with foil tapes and stored at −80° C.

B. Colony Array—Gridding. Membrane filters were soaked in LB/Ampicillin for 10 minutes. Filters were aligned onto fresh 22×22 cm agar plates and allowed to dry on the plates 30 min. in a Laminar flowhood. Plates and filters were placed in the Qbot and UV sterilized for 20 minutes. Following sterilization, plates/filters were gridded from 384 well plates using the Qbot according to the manufacturers specifications with the following parameters:

Gridding Routine

Name: 3×3

Source

Container: Hotel (9 High)

Plate: Genetix Plate 384 Well

Max Plates: 8

Inking time (ms): 1000

Destination

Filter holder: Qtray

Gridding Pattern: 3×3, non-duplicate, 8

Field Order: front 6 fields

No. Filters: up to 15

Max stamps per ink: 1

Max stamps per spot: 1

Stamp time (ms): 1000

No. Fields in Filter: 2

No. Identical Fields: 2

Stamps between sterilize: 1

Head: 384 pin gravity gridding head

Pin Height Adjustment: No change

Qbot Bath #1

Bath cycles: 4

Dry time: 10 (Seconds)

Wait After Drying: 10 (Seconds)

C. Plate Rearray. 384 well plates were rearrayed into deep 96 well block format using the Qbot according to the manufacturers instructions and the following rearray parameters X2 per plate:

Source

Container: Hotel (9 High)

Plate: Genetix Plate 384 Well

1^{st} Plate: 1

Destination

Container: Universal Dest Plate Holder

Plate: Beckman 96 Deep Well Plate

1^{st} plate: 1

Dips to Inoculate: 5

Well offset: 1

Max plates to use: 12 (or less)

Time in wells (sec): 2

Qbot Bath #1

Head: 96 pin picking head

First Picking Pin: 1

Pin Order: A1-H1, A2-H2, A3-H3 . . .

Bathcycles: 4

Sec. In dryer: 10

Wait after drying: 10

Following rearray, the 96-well blocks were covered with airpore tape and placed in incubator shakers at 37° C., 500 rpm for a total of 24 hours. Plates were removed and used for DNA preparation.

### Example 10

### DNA Preparation

Plasmid DNA was prepared in a 96-well block format using a Qiagen Biorobot 9600 instrument (Qiagen, Valencia Calif.) according to the manufacturers specifications. In this 96-well block format, 900 μl of cell lysate was transferred to the Qiaprep filter and vacuumed 5 min. at 600 mbar. Following this vacuum, the filter was discarded and the Qiaprep Prep-Block was vacuumed for 2 min at 600 mbar. After adding buffer, samples were centrifuged for 5 min at 600 rpm (Eppendorf benchtop centrifuge fitted with 96-wp rotor) and subsequently washed X2 with PE. Elution was carried out for 1 minute, followed by a 5 min. centrifugation at 6000 rpm. Final volume of DNA product was approximately 75 μl.

### Example 11

### Generation of Raw Sequence Data and Filtering Protocols

High-throughput sequencing was carried out using the PCT200 and TETRAD PCR machines (MJ Research, Watertown, Mass.) in 96-well plate format in combination with two ABI 377 automated DNA sequencers (PE Corporation, Norwalk, Conn.). The throughput at present is six 96-well plates per day. The quality of sequence data is improved by filtering the raw sequence output from sequencer. One criteria is to make sure that the unreadable bases are less than 10% of the total number of bases for any sequence and that there are no more than ten consecutive Ns in the middle part of the sequence (40-450). The sequences that pass these tests are defined as being of high quality. The second step for improving the quality of a sequence is to remove the vectors from the sequence. There are two advantages of this process. First, when locating the vector sequence, its position can be used to align to the input sequence. The quality of the sequence can be evaluated by the alignment between the vector sequence and the target sequence. Second, the removal of the vector sequence greatly improves the signal-to-noise ratio and makes the analysis of the resulting database search much easier. A third important pre-filtering step is to eliminate the duplicates in a library so it will speed up the analysis and reduce redundant analyses.

### Example 12

### Automated Transcriptions and Encapsidations

Plasmid DNA preparations were subjected to automated transcription reactions in a 96-well plate format using a Tecan Genesis Assay Workstation 200 robotic liquid handling system (Tecan, Inc., Research Triangle Park, N.C.) according to the manufacturers specifications, operating on the Gemini Software (Tecan, Inc.) program “Automated_Txns.gem. For these reactions, reagents from Ambion, Inc. (Austin, Tex.) were used according to the manufacturers specifications at 0.4× reaction volumes. Following the robotic set-up of transcription reactions, 96-well plates were removed from the Tecan, shaken on a platform shaker for 30 sec., centrifuged in an Eppendorf tabletop centrifuge fitted with a 96-well plate rotor at 700 rcf for 1 minute and incubated at 37° C. for 1.5 hours.

During the transcription reaction incubation, encapsidation mixture was prepared according to the following recipe:

| | 1X Solution |
|---|---|
| Sterile ddi H₂O | 100.5 μl |
| 1 M Sodium Phosphate | 13.0 μl |
| TMV Coat Protein (20 mg/ml) | 6.5 μl |
| | 120 μl |
| | per well |

This mixture was placed in a reservoir of the Tecan and added to the 96-well plates containing transcription reaction following the incubation period using Gemini software program “9_Plates.gem”. After adding encapsidation mixture, plates were shaken for 30 sec. on a platform shaker, briefly centrifuged as described above, and incubated at room temperature overnight. Prior to inoculation, encapsidated transcript was sampled and subjected to agarose gel analysis for QC.

### Example 13

### Infection of N. benthamiana Plants with GENEWARE® Viral Transcripts and Plant Growth

N. benthamiana seeds were sown in 6.5 cm pots filled with Redi-earth medium (Scotts) that had been pre-wetted with fertilizer solution (147 kg Peters Excel 15-5-15 Cal-Mag (The Scotts Company, Marysville Ohio), 68 kg Peters Excel 15-0-0 Cal-Lite, and 45 kg Peters Excel 10-0-0 MagNitrate in 596L hot tap H₂O, injected (H. E. Anderson, Muskogee Okla.) into irrigation water at a ratio of 200:1). Seeded pots were placed in the greenhouse for 1 d, transferred to a germination chamber, set to 27° C., for 2 d (Carolina Greenhouses, Kinston, N.C.), and then returned to the greenhouse. Shade curtains (33% transmittance) were used to reduce solar intensity in the greenhouse and artificial lighting, a 1:1 mixture of metal halide and high pressure sodium lamps (Sylvania) that delivered an irradiance of approximately 220 μmol m²s⁻¹, was used to extend day length to 16 h and to supplement solar radiation on overcast days. Evaporative cooling and steam heat were used to regulate greenhouse temperature, maintaining a daytime set point of 27° C. and a nighttime set point of 22° C. At approximately 7 days post sowing (dps), seedlings were thinned to one seedling per pot and at 17 to 21 dps, the pots were spaced farther apart to accommodate plant growth. Plants were watered with Hoagland nutrient solution as required. Following inoculation, waste irrigation water was collected and treated with 0.5% sodium hypochlorite for 10 minutes to neutralize any viral contamination before discharging into the municipal sewer.

### Example 14

### Plant Inoculation

For each GENEWARE® clone, 180 μL of inoculum was prepared by combining equal volumes of encapsidated RNA transcript and FES buffer (0.1M glycine, 0.06 M K₂HPO₄, 1% sodium pyrophosphate, 1% diatomaceous earth (Sigma), and either 1% silicon carbide (Aldrich), or 1% Bentonite (Sigma)). The inoculum was applied to three greenhouse-grown Nicotiana benthamiana plants at 14 or 17 days post sowing (dps) by distributing it onto the upper surface of one pair of leaves of each plant (−30 μL per leaf). Either the first pair of leaves or the second pair of leaves above the cotyledons was inoculated on 14 or 17 dps plants, respectively. The inoculum was spread across the leaf surface using one of two different procedures. The first procedure utilized a Cleanfoam swab (Texwipe Co, N.J.) to spread the inoculum across the surface of the leaf while the leaf was supported with a plastic pot label (¾×5 2M/RL, White Thermal Pot Label, United Label). The second implemented a 3″ cotton tipped applicator (Calapro Swab, Fisher Scientific) to spread the inoculum and a gloved finger to support the leaf. Following inoculation the plants were misted with deionized water and maintained in the greenhouse.

At 13 days post inoculation (dpi), the plants were examined visually and a numerical score was assigned to each plant to indicate the extent of viral infection symptoms. 0=no infection, 1=possible infection, 2=infection symptoms limited to leaves <50-75% fully expanded, 3=typical infection, 4=atypically severe infection, often accompanied by moderate to severe wilting and/or necrosis.

### Example 15

### Phenotype Assay

At 13 dpi plants were examined and in cases where a plant's visual phenotype deviated substantially from the phenotypes of control plants, a controlled vocabulary utilizing a five-part phrase was used to describe the plants. Phrase: plant region/sub-part/modifier (optional)/symptom/severity. Plant regions: sink leaves (the upper region of the plant considered to be primarily phloem sink tissue at the time of evaluation), source leaves (expanded, fully-infected leaves considered to be phloem source tissue at the time of evaluation), bypassed leaves (leaves directly above inoculated leaves that display little or no infection symptoms), inoculated leaves (leaves one and two on 14 dps-inoculated plants or leaves three and four on 17 dps-inoculated plants), stem. Subparts: blade, entire, flower, foci, intervein, leaf, major vein, margin, minor vein, node, petiole, shoot apex, upper, vein, viral path. Modifiers: apical, associated, banded, basal, blotchy, bright, central, crinkled, dark, epinastic, flecked, glossy, gray, hyponastic, increased, intermittent, large-spotted, light, light-colored, light-green, mottled, narrowed, orange, patchy, patterned, radial, reduced, ringspot, small-spotted, smooth, spotted, streaked, subtending, uniform, unusual, white. Symptoms: bleaching, chlorosis, color, contortion, corrugation, curling, dark green, elongation, etching, hyperbranching, mild symptoms, necrosis, patterning, recovery, stunting, texture, trichomes, wilting. Severity: 1—extremely mild/trace, 2—mild symptom (<30% of subpart affected), 3—moderate symptom (30%-70% of subpart affected), 4—severe symptom (>70% of subpart affected). Based on the symptoms a phenotypic hit value (PHV) and a herbicide hit value (HHV) were assigned to each plant phenotyped. Phenotype Hit Value: 1—no predicted value; do not request for repeat analysis, 2—of uncertain value, 3—of potential value; strong phenotype, 4—highly unusual phenotype. Herbicide Hit Value: 1—no predicted value; do not request for repeat analysis, 2—of uncertain value, 3—moderate chlorosis (especially in apical region) or necrosis, 4—Severe phytotoxicity/herbicide mode of action. Comments were added if additional information was required to complete the plant characterization.

Phenotypic data was tabulated on worksheets and entered into the database. Phenotypic hits were identified two ways. Using the phenotypic hit value and herbicide hit value to generate a list and performing a database query for selected symptoms. Clones designated as hits were identified and rearrayed from master 384-well plates of frozen E. coli glycerol stocks using a Tecan Genesis RSP200 device fitted with a ROMA arm, according to the manufacturers specifications and operating on Gemini software (Tecan) program “worklist.gem” according to instructions downloaded from a proprietary LIMS program (LSBC Inc., Vacaville, Calif.). DNA clones were rearrayed and the DNA preparation, transcription, encapsidation, inoculation and screen procedure was repeated. After phenotyping was complete the database was queried for each hit seeking complete phenotypic descriptions. The results of the query were analyzed and plants displaying symptoms strongly or in replication were selected for further analysis. As a result of the analysis, the hits were segregated into positive phenotypic hit (confirmed hit) and negative phenotypic hit categories. Plants in the negative hit category failed to express a defined phenotype reproducibly; plants in the positive hit category expressed a defined phenotype reproducibly (data for positive hits shown in FIG. 6). Homologs to hit sequences were screened similarly (data shown in FIG. 7). The following definitions were used in the evaluation of reproducibility.

### Definitions

Severity

1—extremely mild/trace

2—mild symptom (<30% of subpart affected)

3—moderate symptom (30%-70% of subpart affected)

4—severe symptom (>70% of subpart affected)

Symptom: A visual condition resulting from the action of the GENEWARE® vector or the clone insert.

Visual phenotype: A plant displaying a symptom or group of symptoms that meet defined criteria.

Stunting: Stunting is considered present as a phenotype when any stunting symptoms are present in any plant part. Stunting symptoms include reduced internodal length, reduced petiole length, reduced shoot apex length and reduced leaf blade diameter (along two axis). Other symptoms that are typically viral such as mild (level 2 severity code) chlorosis and blade curling may be present as well. If any additional symptoms such as necrosis, wilting or etching are present (excluding the inoculated leaves) at any level the plant does not fit the criteria for a stunting phenotype.

Chlorosis: Chlorosis is considered present as a phenotype when chlorotic symptoms are present in any plant part. Chlorosis is a loss or reduced development of chlorophyll. This typically creates a yellow to light green pigmentation. Other symptoms that are typically viral such as blade curling may be present as well. If any additional symptoms such as necrosis, wilting or etching are present (excluding the inoculated leaves) above a severity level 1 the plant does not fit the criteria for a chlorotic phenotype.

Bleaching; Bleaching is considered present as a phenotype when bleaching symptoms are present in any plant part. Bleaching is the loss of all pigment resulting in a leaf with a white appearance. This loss of pigmentation does not result in a loss of turgor. Other symptoms that are typically viral such as mild chlorosis and blade curling may be present as well. When additional symptoms (such as necrosis, wilting or etching) are present bleaching symptoms must be present above a severity level 1 to fit the criteria for a bleaching phenotype. If any additional symptoms such as necrosis, wilting or etching are present (excluding the inoculated leaves) above a severity level 2 the plant does not fit the criteria for a bleaching phenotype.

Etching: Etching is considered present as a phenotype when etching symptoms are present in any plant part. Etching is necrosis of epidermal cells. Other symptoms that are typically viral such as mild chlorosis and blade curling may be present as well. When additional symptoms (such as necrosis or wilting) are present etching symptoms must be present above a severity level 1 to fit the criteria for an etching phenotype. If any additional symptoms such as necrosis or wilting are present (excluding the inoculated leaves) above a severity level 1 the plant does not fit the criteria for an etching phenotype.

Wilting: Wilting is considered present as a phenotype when wilting symptoms are present in any plant part. Wilting is the loss of turgor. Other symptoms that are typically viral such as mild chlorosis and blade curling may be present as well. When additional symptoms (such as necrosis or etching) are present wilting symptoms must be present above a severity level 1 to fit the criteria for a wilting phenotype. If any additional symptoms such as necrosis or etching are present (excluding the inoculated leaves) above a severity level 1 the plant does not fit the criteria for a wilting phenotype.

Necrosis: Necrosis is considered present as a phenotype when necrotic symptoms are present in any plant part. Necrosis is the death of tissue. When bleaching symptoms are present necrotic symptoms must be present above a severity level 2 to fit the criteria for a necrosis phenotype. In all other plants when necrosis is present above a severity level 1 the plant fits the criteria for a necrosis phenotype.

Auxin Response: Auxin response phenotype is considered present as a phenotype when auxin response symptoms are present in any plant part (except as noted). Auxin response symptoms are petiole or stem curling, bleaching, chlorosis, wilting, stunting and necrosis. Petiole or stem curling must be present in all cases for the plant to fit the criteria of the auxin response phenotype. All other symptoms may not be present in all cases. Necrosis in the petiole or stem may not be present at any level for the plant to fit the criteria for the auxin response phenotype.

Chlorosis/Etching: Chlorosis/etching phenotype is considered present as a phenotype when chlorosis and etching symptoms are present in any plant part. Chlorosis symptoms must be present above a severity level 2 and etching symptoms must be present above a severity level 1 for a plant to fit the criteria for a chlorosis/etching phenotype. Other symptoms that are typically viral such as blade curling may be present as well. If any additional symptoms such as necrosis or wilting are present (excluding the inoculated leaves) above a severity level 1 the plant does not fit the criteria for a chlorosis/etching phenotype.

Mixed: Mixed is a phenotype that is typified by a consistent expression of a group of symptoms in a group of plants inoculated by the same clone. Other symptoms that are typically viral such as mild chlorosis and blade curling may be present as well. If there are any additional symptoms present not consistently expressed in the group of plants (excluding the inoculated leaves) above a severity level 1 the plants do not fit the criteria for a mixed phenotype.

Multiple Phenotype: Multiple phenotype is considered present as a phenotype when more than one phenotype is present for the same clone but no phenotype has a reproducibility >49%.

Other: A symptom or group of symptoms that do not meet the criteria for a defined phenotype (example: same plant displays wilting and stunting).

Dark Green: Dark green is considered present as a phenotype when dark green symptoms are present in any plant part. Dark green is the increased presence of green pigment. Other symptoms that are typically viral such as mild chlorosis and blade curling may be present as well. Texture may be present at a severity level 2 or less and stunting may be present at any level. If any additional symptoms such as necrosis, wilting or etching are present (excluding the inoculated leaves) above a severity level 1 the plant does not fit the criteria for a dark green phenotype.

Gray Leaf: Gray leaf is considered present as a phenotype when gray leaf symptoms are present in any plant part. Gray leaf is the presence of gray, dark gray, gray dark green or light gray pigment. Stunting may be present at any level. Other symptoms that are typically viral such as mild chlorosis and blade curling may be present as well. If any additional symptoms such as necrosis, wilting or etching are present (excluding the inoculated leaves) above a severity level 1 the plant does not fit the criteria for a gray leaf phenotype.

Wet Leaf: Wet leaf is considered present as a phenotype when wet leaf symptoms are present in the leaf blade. Wet leaf is the presence of moisture (glossy texture symptom) on the leaf blade surface. Other symptoms include vein, mottled or blotchy chlorosis, blotchy necrosis, etching, dark green and blade curling. Stunting may be present at any level. All symptoms do not need to be present.

Elongation: Elongation is considered present as a phenotype when elongation symptoms are present in any plant part. Elongation symptoms include increased internodal length, increased petiole length and increased shoot apex length. Other symptoms that are typically viral such as mild chlorosis and blade curling may be present as well. If any additional symptoms such as necrosis, wilting or etching are present (excluding the inoculated leaves) above level 1 the plant does not fit the criteria for an elongation phenotype.

Fluorescent: Fluorescence is considered present as a phenotype when any plant part is fluorescent under UV light. Fluorescent symptoms include the presence of blue or blue gray fluorescent pigments. Other symptoms that are typically viral such as blade curling may be present as well. Chlorosis and stunting may be present at any level. If any additional symptoms such as necrosis, wilting or etching are present (excluding the inoculated leaves) above level 1 the plant does not fit the criteria for a fluorescent phenotype.

Texture: Texture is considered present as a phenotype when texture symptoms are present in the leaf blade. Texture is the presence of an increased level of rough or pebbly leaf surface features. Other symptoms that may be present at any level are glossy texture (wet leaf), corrugation, curling, chlorosis and stunting. If necrosis, wilting or etching are present (excluding the inoculated leaves) above level 1 the plant does not fit the criteria for a texture phenotype.

Gray Leaf/Wet Leaf: Gray Leaf/Wet Leaf is considered present as a phenotype when both gray leaf symptoms and wet leaf symptoms are present in the leaf blade. Other symptoms include vein, mottled or blotchy chlorosis, blotchy necrosis, etching, dark green and blade curling. Stunting may be present at any level.

### Example 16

### Bioinformatic Analysis of Hits

A. Phred and Phrap. Phred is a UNIX based program which can read DNA sequencer traces and make nucleotide base calls independent of any software provided by the DNA sequencer manufacturer. Phred also provides a quality score for each base that can be used by the investigator to trim those sequences or preferably by Phrap to help its assembly process.

Phrap is another UNIX based program which takes the output of Phred and tries to assemble the individual sequencing runs into larger contiguous segments on the assumption that they all belong to a single DNA molecule. While this is clearly not the case with collections of Expressed Sequence Tags (ESTs) or with heterogeneous collections of sequencing runs belonging to more than one contiguous segment, the program does a very good job of uniquely assembling these collections with the proper manipulation of its parameters (mainly -penalty and -minscore; settings of 15 and 40 respectively provide contiguous sequences with exact homology approaching 95% over lengths of approximately 50 nucleotide base pairs or more). As with all assemblies it is possible for proper assemblies to be missed and for improper assemblies to be constructed, but the use of the above parameters and judicious use of input sequences will keep these to a minimum.

Detailed descriptions of the Phred and Phrap software and it's use can be found in the following references which are hereby incorporated herein by reference: Ewing et al., Genome Res. 8:175 [1998]; Ewing & Green, Genome Res. 8:186 [1998]; Gordon, D., C. Abajian, and P. Green., Genome Res. 8:195 [1998].

Blast

The BLAST set of programs may be used to compare a set of sequences against databases composed of large numbers of nucleotide or protein sequences and obtain homologies to sequences with known function or properties. Detailed description of the BLAST software and its uses can be found in the following references which are hereby incorporated herein by reference: Altschul et al., J. Mol. Biol. 215:403 [1990]; Altschul et al., J. Mol. Biol. 219:555 [1991].

Generally, BLAST performs sequence similarity searching and is divided into 5 basic subroutines of which 3 were used: (1) BLASTN compares a nucleotide sequence to a nucleic acid sequence database; (2) BLASTX compares translated protein sequences from a nucleotide sequence done in six frames to a protein sequence database; (3) TBLASTX compares translated protein sequences from a nucleotide sequence done in six frames to the six frame translation of a nucleotide database. BLASTX and TBLASTX are used to identify homologies at the protein level of the nucleotide sequence.

B. Contig Sequence Assembly for Hits. Phred sequence calls and quality data for the individual sequencing runs associated with SEQ ID NOs 1-311 (FIG. 1) were stored in a relational database. All the sequence runs stored in the database for the sequences to be assembled were extracted from the database and the files needed by Phrap recreated with the aid of a Perl script. Perl is an interpreted computer language useful for data manipulation. The same script ran Phrap on the assembled files and then stored the assembled contiguous sequences and singletons in a relational database. The script then assembled two files. One file was a FASTA format file of the sequences of the assembled contigs and singletons (FIG. 1). The other file was a record of the assembled sequences and which sequencing runs they contained (data not shown). FASTA format is a standard DNA sequence format recognized by the BLAST suite of programs as well as by Phrap. Both of these files were then inspected manually to detect incorrect assemblies or to add sequence information not present in the relational database. Any incorrect assemblies found were corrected before this file was used in BLAST searches to identify function and well as other homologous sequences in our databases. Correct assemblies that contained more than one SEQ ID were separated. Although these represent parts of the same sequence, since these are ESTs and contain limited gene sequence data, a one-to-one nucleotide match cannot be predicted at this time for the entire length of a contig representing a single SEQ ID with those containing multiple SEQ IDs. Some full length sequences were obtained and are designated with a FL.

C. Identification of Function. The FASTA formatted file obtained as described above was used to run a BLASTX query against the GenBank non-redundant protein database using a Perl script. The data from this analysis was parsed out by the Perl script such that the following information was extracted: the query sequence name, the level of homology to the hit and the description of the hit sequence (the highest scoring hit from the analysis). The script filtered all hits less than 1.00E-04, to eliminate spurious homologies. The data from this file was used to identify putative functions and properties for the query sequences (see FIG. 3).

D. Identification of Similar Sequences in Derwent. The FASTA formatted file obtained as described above was used to run a BLASTN query against the Derwent non-redundant nucleotide database as well as a BLASTX against the Derwent non-redundant protein database using Perl scripts. These Derwent non-redundant databases were created by extracting all the sequence information in the Derwent database. The data from this analysis was parsed out by the Perl script such that the following information was extracted, the query sequence name, the level of homology to the hit and the description of the hit sequence (the highest scoring hit from the analysis). The script filtered all hits less than 1.00E-04, to eliminate spurious homologies (see FIGS. 4 and 5)

E. Identification of Homologous Sequences. eBRAD, an internal relational database, stored sequence data and results from biological and metabolic screens of multiple organisms (Nicotiana benthamiana, Oryzae sativa (var. Indica IR7), Papaver rhoeas, Saccharomyces cerevisiae and Trichoderma harzianum (Rifai 1295-22)). In order to identify sequences in the database with high levels of homology to the sequences functionally identified as “hits” and contained in the FASTA formatted file described above, the following analysis was performed.

All the sequences were extracted in FASTA format from the eBRAD relational database with standard SQL commands and converted into a searchable BLAST database using tools provided in the BLAST download from the National Center for Biotechnology Information (NCBI). A Perl script then ran a BLASTN search of our query file against the eBRAD database containing all relevant sequences. The script then extracted from all hits the following information: the query name, the level of homology and the identity of the hit sequences. The script then filtered all homologies less than 1.00E-20 as well as all the redundant hit sequences.

This analysis was repeated again using a TBLASTX query. Both files were then combined and the redundancies eliminated. Since the query sequences are also present in the database, those query sequences were eliminated as redundant.

These results were used to extract the sequence and quality score data from the eBRAD relational database in order to repeat the analysis described in “Contig Sequence Assembly for Hits” (except that contig assemblies from the same organism were permitted to be comprised of independently cloned, but overlapping, sequences). FIG. 2 provides the assembled search hits with homologies better than 1.00E-20 to the sequences shown in FIG. 1.

### Example 17

### Preparation and Transformation of Arabidopsis thaliana

A. Growth Conditions: Seed Preparation for Sowing

Freshly harvested seed was allowed to dry for 7 days at room temperature in the presence of desiccant. Dried seed was sterilized with a 0.1% Triton X-100 (Sigma Chemical Co., St. Louis, Mo.) and 70% ethanol solution (3 minutes) using 95% ethanol (30 seconds) as a wash. After sterilization, seed was suspended in a 0.1% Agarose (Sigma Chemical Co., St. Louis, Mo.) solution. The suspended seed was stored at 4° C. for 2 days to complete dormancy requirements and ensure synchronous seed germination (stratification).

Sowing

Sunshine Mix LP5 (Sun Gro Horticulture Inc., Bellevue, Wash.) was covered with fine vermiculite and sub-irrigated with Hoaglan's solution until wet. The soil mix was allowed to drain for 24 hours. Stratified seed was sown onto the vermiculite and covered with humidity domes (KORD Products, Bramalea, Ontario, Canada) for 7 days.

Growth Conditions

Seeds were germinated and plants were grown in a Conviron (models CMP4030 and CMP3244, Controlled Environments Limited, Winnipeg, Manitoba, Canada) under long day conditions (16 hours light/8 hours dark) at a light intensity of 120-150 μmol/m2sec under constant temperature (22° C.) and humidity (40-50%). Plants were initially watered with Hoaglan's solution and subsequently with DI water to keep the soil moist but not wet. Plants nearing seed harvest (1-2 weeks before harvest) were allowed to dry out.

B. Gene Subcloning

ORFs from genes of interest were excised from GENEWARE® (Large Scale Biology, Vacaville, Calif.) and inserted into binary vectors using one of the two methods outlined below.

a. Method A (pENTR/D-TOPO® Method (Invitrogen, Carlsbad, Calif.))

i. PCR Primer Design

PCR primers for directional cloning into the standard pENTR/D-TOPO® vector (Invitrogen, Carlsbad, Calif.) were designed as follows:

```
          Sense Primer PacI Insert
    ˜˜˜˜˜˜˜˜˜˜˜˜˜˜˜˜˜˜˜˜˜˜˜˜˜˜˜˜ ˜˜˜˜˜˜˜˜˜ ˜˜˜˜˜˜˜˜
5′ CACCATCTCA GTTCGTGTTC TTGTCATTAA TTAA gtgcccggg
    Insert NotI Vector
    ˜˜˜˜˜˜˜˜ ˜˜˜˜˜˜˜˜ ˜˜˜˜˜˜˜˜˜˜˜˜˜˜˜˜˜˜˜˜˜˜˜˜˜
gcaaaaaaaa GCGGCCGCG TCGAGGGGTA GTCAAGATGC . . . SEQ ID NO 2072
                Antisense Primer
      ˜˜˜˜˜˜˜˜˜˜˜˜˜˜˜˜˜˜˜˜˜˜˜
. . . GTG TCCGTAATCA CACGTGGTGC 3′ SEQ ID NO:2073
```

ORF's corresponding and were cloned into the pENTR/D-TOPO® vector as per manufacturer's instructions. The PCR reaction was completed using Platinum Pfx DNA polymerase from Invitrogen at 0.5× concentration. PCR reactions were carried out in a MJ Research Peltier Thermal Cycler programmed with the following conditions; 1) 94° C. for 2 minutes 2) 94° C. for 15 seconds 3) 55° C. for 30 seconds 4) 68° C. for 2 minutes 5) 18 times to step 2. The reaction was maintained at 4° C. after cycling. The amplification was analyzed by 1% agarose gel electrophoresis and visualized by ethidium bromide staining. A clean band of expected size was verified and extracted using a Qiaex II Gel Extraction Kit (Qiagen Inc, Valencia, Calif.).

ii. TOPO® Cloning and DH5α Transformation

A TOPO® Cloning reaction was carried out as follows; 4 μl of purified PCR product, 1 μl salt solution (provided in kit) and 1 μl of TOPO® vector were combined, mixed gently and incubated for 5 minutes at room temperature. After incubation the reaction was placed on ice, then transformed into competent E. coli MaxEfficiency DH5α cells (Invitrogen) using the heatshock method. 2 μl of the TOPO® cloning reaction and 50 μl of DH5α cells were mixed gently and incubated on ice for 30 minutes. The cells were then heat-shocked for 30 seconds at 42° C. without shaking and immediately transferred to ice. 250 μl of room temperature SOC medium was added. The reaction was then incubated at 37° C. for 1 hour with constant agitation. Various amounts of the culture was spread onto LB+agar plates containing kanamycin (Sigma Chemical Co., St. Louis, Mo.) (50 mg/L) and incubated overnight at 37° C.

iii. Qiagen Spin Mini Preps (Valencia, Calif.) and Sequencing

After overnight incubation visible colonies were selected and streaked out onto a fresh LB+agar with kanamycin plate and allowed to incubate for 6-12 hours. These colonies were used to inoculate 4 mL mini prep cultures (liquid LB+kanamycin). The cultures were incubated overnight at 37° C. with constant agitation. Qiagen (Valencia, Calif.) Spin Mini Preps, performed per manufacturer's instructions, were used to purify the plasmid DNA. The DNA recovered was digested with the PacI, XhoI and NotI enzymes and the digests were analyzed by 1% agarose gel electrophoresis and visualized by ethidium bromide staining to check for correct insert size and orientation. The plasmid DNA from the midi preps was sequenced using primers (M13 and M13R) supplied with the TOPO® cloning kit verifying that the desired fragments were present and in the correct orientation. Additional sequencing using gene sequence specific primers was carried out to ensure that the plasmid DNA did not contain any PCR derived mutations. A DNA Sequencing Kit (Applied Biosystems, Foster City, Calif.) was used with the following reaction mix; 1 μl DNA (0.5 μg of DNA), 1 μl 3.2 μM primer, 1 μl DMSO (supplied with kit), 6 μl Big Dye™ready reaction mix (supplied with kit) and sterile water to 15 μl. Sequencing reactions were carried out in a MJ Research Peltier Thermal Cycler programmed with the following conditions; 1) 95° C. for 20 seconds 2) 50° C. for 20 seconds 3) 60° C. for 4 minutes 4) 29 times to step 2. The reaction was maintained at 4° C. after cycling.

iv. Cloning into the Binary Vector

The binary vector pMYC3446 (Dow AgroSciences, Indianapolis, Ind.) has been modified to include recombination sites utilized in Gateway™ (Invitrogen, Carlsbad, Calif.) cloning. The recombination reaction mix was assembled as follows; 4 μl LR reaction buffer (included in kit), 1.4 μl of entry clone (300 ng of DNA of desired fragment in pENTR/D-TOPO), 1 μl (300 ng) of destination vector (pMYC3446), TE buffer to 16 μl, 4 μl of Gateway™ LR Clonase™ Enzyme Mix (included in kit). This reaction was allowed to proceed for 3 hours at room temperature. To stop the reaction, 2 μl of Proteinase K solution (included in kit) was added, and the reaction was incubated at 37° C. for 10 minutes. 1 μl of the clonase reaction was used to transform MaxEfficiency DH5α cells. The transformation followed the same protocol as above with the following exceptions, 450 μl SOC was added after heat-shock and the reaction was incubated for 3.5 hours. The cells were plated on LB+agar plates containing spectinomycin (Sigma Chemical Co., St. Louis, Mo.) (100 mg/L) and allowed to incubate overnight at 37° C. The protocol for Qiagen Spin Mini Preps (Valencia, Calif.) and DNA digest was followed as described above with the following exception, spectinomycin (100 mg/L) was used for selection. After electrophoresis one of the colonies with the correct insert size and orientation was selected for Agrobacterium and Arabidopsis transformation as described below.

b. Method B (Modified pENTR/D-TOPO® Method)

i. pENTR/D-TOPO® Modification

The pENTR/D-TOPO® vector was modified with a PCR product to include a restriction endonuclease cloning site for the enzymes PacI and XhoI between the attL1 and attL2 recognition sites. Primers were designed to PCR amplify a region of DNA that would include the directional cloning sequence for standard pENTR/D-TOPO cloning (a CACC inserted at the 5′ end of the PCR product), and would also include the PacI (5′) and XhoI (3′) restriction sites (see primer design above). PCR amplification, TOPO® cloning, DH5α transformation and DNA purification and digest (with PacI and XhoI only) were all performed per the protocols above. After electrophoresis verified that the correct band size for the vector was present the TOPO® vector band was extracted and gel purified as described above.

ii. Ligation and DH5α Transformation

ORFs were ligated into the modified pENTR/D-TOPO® cloning vector. When an ORF did not have a 3′ XhoI site (a NotI site was present on the 3′ end) one was added using a vector (pYES2) with an XhoI site on the 3′ side of a NotI site.

The desired fragment and the modified pENTR/D-TOPO® vector were ligated together using T4 ligase (Invitrogen). The following components were mixed together in a 1.5 mL eppendorf tube: 5 μl DNA fragment, 2 μl modified TOPO® vector, 2 μl 5× ligation buffer (included with kit) and 1 μl T4 ligase (included with kit). This ligation was placed into a 16° C. water bath and allowed to react overnight. The ligation was transformed into DH5a cells as described above. DNA purification, sequencing (M13 and M13R only) and Gateway cloning were performed as described above. Agrobacterium and Arabidopsis transformation were performed as described below.

C. Agrobacterium Transformation-Electroporation

Electro-competent Agrobacterium tumefaciens (strain Z707S) cells were prepared using a protocol from Weigel and Glazebrook (2002). The competent agro cells were transformed using an electroporation method adapted from Weigel and Glazebrook (2002). 50 μl of competent agro cells were thawed on ice and 10-25 ng of the desired plasmid was added to the cells. The DNA and cell mix was added to pre-chilled electroporation cuvettes (2 mm). An Eppendorf Electroporator 2510 was used for the transformation with the following conditions, Voltage: 2.4 kV, Pulse length: 5 msec. After electroporation, 1 mL of YEP broth was added to the cuvette and the cell-YEP suspension was transferred to a 15 ml culture tube. The cells were incubated at 28° C. in a water bath with constant agitation for 4 hours. After incubation, the culture was plated on YEP+agar with spectinomycin (100 mg/L) and streptomycin (Sigma Chemical Co., St. Louis, Mo.) (250 mg/L). The plates were incubated for 2 days at 28° C. Colonies were selected and streaked onto fresh YEP+agar with spectinomycin (100 mg/L) and streptomycin (250 mg/L) plates and incubated at 28° C. for 1 day. Colonies were selected for PCR analysis to verify the presence of the gene insert by using vector specific primers. A small scraping of cells was diluted into 10 μl water. The cells were lysed at 100° C. for 5 minutes and directly amplified. Plasmid DNA from the binary vector used in the agro transformation was included as a control. The PCR reaction was completed using Taq DNA polymerase from Invitrogen per manufacture's instructions at 0.5× concentrations. PCR reactions were carried out in a MJ Research Peltier Thermal Cycler programmed with the following conditions; 1) 94° C. for 3 minutes 2) 94° C. for 45 seconds 3) 55° C. for 30 seconds 4) 72° C. for 1 minute 30 seconds 5) 29 times to step 2 6) 72° C. for 10 minutes. The reaction was maintained at 4° C. after cycling. The amplification was analyzed by 1% agarose gel electrophoresis and visualized by ethidium bromide staining. A colony was selected whose PCR product was identical to the plasmid control.

D. Arabidopsis Transformation-Floral Dip Method

Arabidopsis was transformed using the floral dip method from Weigel and Glazebrook (2002). The selected colony was used to inoculate a 400 mL culture of YEP broth containing spectinomycin (100 mg/L) and streptomycin (250 mg/L), and the culture was incubated overnight at 28° C. with constant agitation. The cells were then pelleted at approx. 8700×g for 15 minutes, and the resulting supernatant discarded. The cell pellet was gently resuspended in 400 mL infiltration media as prescribed by Weigel and Glazebrook (2002) with the following exception, 1/2× Gamborg's was used. Plants approximately 1 month old were dipped into the media for 30 seconds, being sure to submerge the newest influorescences. The plants were then laid down on their sides and covered for 24 hours, then lightly misted with water to rinse, and placed upright. The plants were grown at 22° C., with a 16-hour light/8-hour dark photoperiod. Approximately 3 weeks after dipping, the seeds were harvested.

Selection of Transformed Plants

T1 seed was sown on 10.5″×21″ germination trays (T.O. Plastics Inc., Clearwater, Minn.) as described and grown under the conditions outlined. 5-6 days post sowing the domes were removed and plants were sprayed with a 1000× solution of Finale (5.78% glufosinate ammonium, Farnam Companies Inc., Phoenix, Ariz.). Two subsequent sprays were performed at 5-7 day intervals. Survivors (plants actively growing) were identified 7-10 after the final spraying and transplanted into pots prepared with Sunshine mix LP5. Transplanted plants were covered with a humidity dome for 3-4 days and placed in a Conviron with the above mentioned growth conditions.

*D. Weigel, J. Glazebrook. 2002. Arabidopsis: A Laboratory Manual. Cold Spring Harbor Laboratory Press.

### Example 18

### Arabidopsis DNA Isolation (DNeasy Kit, Qiagen)

Grind 100 mg of fresh leaf tissue under dry ice to a fine powder using a mortar and pestle. Transfer the tissue powder a to cooled (on dry ice) 2 ml microcentrifuge tube. Do not allow the sample to thaw. Add 400 μl of Buffer AP1 and 4 μl of RNase A stock solution (100 mg/ml) and vortex vigorously. No tissue clumps should be visible. Vortex or pipette further to remove any clumps. Do not mix Buffer AP1 and RNase A prior to use. Incubate the mixture for 45 min at 65° C. Mix periodically during incubation by inverting tube. Add 130 μl of Buffer AP2 to the lysate, mix and incubate for 5 min on ice. Centrifuge the lysate for 5 min at 14,000 rpm. Apply the lysate to the QIAshredder spin column (lilac) sitting in a 2 ml collection tube and centrifuge for 2 min at 14,500 rpm. Transfer flow-through fraction to a new tube without disturbing the cell-debris pellet. Typically 450 μl of lysate is recovered. Add 1.5 volumes of Buffer AP3/E to the cleared lysate and mix by pipetting. It is important to pipette Buffer AP3/E directly onto the cleared lysate and to mix immediately. Apply 650 μl of the mixture, including any precipitate that may have formed, to the DNeasy (Qiagen) mini spin column sitting in a 2 ml collection tube. Centrifuge for 1 min at 8000 rpm and discard the flow-through. Repeat with remaining sample. Discard flow-through and collection tube. Place DNeasy column in a new 2 ml collection tube add 500 μl Buffer AW to the DNeasy column and centrifuge for 1 min at 8000 rpm. Discard flow-through. Add 500 μl Buffer AW to the DNeasy column and centrifuge for 2 min at maximum speed to dry the membrane. Discard flow-through and collection tube. Transfer the DNeasy column to a 1.5 ml microcentrifuge tube and pipette 100 μl of preheated (65° C.) Buffer AE directly onto the DNeasy membrane. Incubate for 5 min at room temperature and then centrifuge for 1 min at 8000 rpm to elute. Repeat elution once as described.

PCR of ORF's from Arabidopsis DNA

Ti's were selected as described. DNA was isolated per the above protocol. The PCR reaction was completed using Taq DNA polymerase from Invitrogen at 0.5× concentration. PCR reactions were carried out in a MJ Research Peltier Thermal Cycler programmed with the following conditions; 1) 94° C. for 3 minutes 2) 95° C. for 45 seconds 3) 55° C. for 30 seconds 4) 72° C. for 1.5 minutes 5) 29 times to step 2. 6) 72° C. for 10 minutes. The reaction was maintained at 4° C. after cycling. The amplification was analyzed by 1% agarose gel electrophoresis and visualized by ethidium bromide staining.

### Primer Design

Vector Sense Primer Vector ORF
˜˜˜˜˜ ˜˜˜˜˜˜˜˜˜˜˜˜˜˜˜˜˜˜˜˜˜˜˜˜˜˜˜˜˜˜˜˜˜ ˜˜˜˜˜ ˜˜˜˜˜˜˜˜
5′ TAAGGAACCA AGTTCGGCAT TTGTGAAAAC SEQ ID NO:2074
Vector Antisense Primer Vector
˜˜˜˜˜ ˜˜˜˜˜˜˜˜˜˜˜˜˜˜˜˜˜˜˜˜˜˜˜˜˜˜˜˜˜˜˜˜˜ ˜˜˜˜˜˜˜˜
CCCCATATGC AGGAGCGGAT CATTCATTGT 3′ SEQ ID NO:2075

### Example 19

### Phenotypic Screen Arabidopsis Results

The ORF corresponding to GBSG0000138039 (SEQ ID NO:2029) was sub-cloned using Method B (Modified pENTR/D-TOPO® Method) and Arabidopsis plants were transformed as described above. T1 plants were selected as described above. Seventy-eight (78) T1 plants were screened for the Fluorescent phenotype using long wave 366NM UV light. Ten (10) of the T1 plants displayed the Fluorescent phenotype. DNA was isolated (as described above) from a sample of ten (10) T1 plants (3 with the Fluorescent phenotype and 7 without the Fluorescent phenotype). PCR was performed as described above. The PCR reaction confirmed the presence of the ORF corresponding to GBSG0000138039 (SEQ ID NO:2029) in all 10 samples.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with particular preferred embodiments, it should be understood that the inventions claimed should not be unduly limited to such specific embodiments. Indeed, various modifications of the described modes for carrying out the invention which are obvious to those skilled in the art and in fields related thereto are intended to be within the scope of the following claims.

1. An isolated nucleic acid selected from the group consisting of SEQ ID NOs: 1-2065 and nucleic acid sequences that hybridize to any thereof under conditions of low stringency, wherein expression of said isolated nucleic acid in a plant results in an altered visual phenotype.

2. A vector comprising the isolated nucleic acid of claim 1.

3. The vector of claim 2, wherein said isolated nucleic acid is operably linked to a plant promoter.

4. A vector according to claim 2, wherein said isolated nucleic acid is in sense orientation.

5. A vector according to claim 2, wherein said isolated nucleic acid is in antisense orientation.

6. A plant transfected with an isolated nucleic according to claim 1.

7. A seed from the plant of claim 6.

8. A leaf from the plant of claim 6.

9. An isolated nucleic acid according to claim 1, for use in conferring an altered visual phenotype.

10. A method for making a transgenic plant comprising:
a. providing a vector according to claim 2 and a plant,
b. and transfecting said plant with said vector.

11. A process for providing an altered visual phenotype in a plant comprising:
a. providing a vector according to claim 2 and a plant,
b. and transfecting said plant with said vector under conditions such that an altered visual phenotype is conferred by expression of said isolated nucleic acid from said vector.

12. An isolated nucleic acid selected from the group consisting of SEQ ID NOs: 1-2065 and nucleic acid sequences that hybridize to any thereof under conditions of low stringency for use in producing a plant with an altered visual phenotype.

13. Cancelled.
